(19) United States
(12) Patent Application Publication
(10) Pub. No.: US 2026/0042788 A1
(43) Pub. Date: Feb. 12, 2026

(54) STAT6 COMPOUNDS

(71) Applicant: Gilead Sciences, Inc., Foster City, CA (US)

(72) Inventors: Alexander J. Burckle, Hayward, CA (US); Elbert Chin, San Mateo, CA (US); Yifan Deng, Milpitas, CA (US); Deeba Ensan, Toronto (CA); Joshua D. Farr, Mountain View, CA (US); Mary Stuart Herlihy, San Francisco, CA (US); Benjamin J. June, San Francisco, CA (US); Jagadeesh N. Manda, Santa Clara, CA (US); Gregory T. Notte, Redwood City, CA (US); Eric Q. Parkhill, Union City, CA (US); Dustin S. Siegel, Half Moon Bay, CA (US); Katie A. Spence, San Francisco, CA (US); Zheng-Yu Yang, Palo Alto, CA (US); Xianhuang Zeng, Redwood City, CA (US)

(21) Appl. No.: 19/272,227

(22) Filed: Jul. 17, 2025

Related U.S. Application Data
(60) Provisional application No. 63/673,485, filed on Jul. 19, 2024.

(51) Int. Cl. C07F 9/6561; A61K 31/675; C07F 9/6558

(52) U.S. Cl. CPC C07F 9/65615; A61K 31/675; C07F 9/65586

(57) ABSTRACT

Modulators of signal transducer and activator of transcription 6 (STAT6) are provided, including compounds of Formula J, I and Ia, pharmaceutical compositions thereof, and methods of treating an inflammatory condition or disease.

### CROSS-REFERENCE TO RELATED APPLICATIONS

This application claims the benefit of priority to U.S. Provisional Application No. 63/673,485, filed Jul. 19, 2024, which is incorporated herein in its entirety for all purposes.

### BACKGROUND

The Signal transducer and activator of transcription 6 (STAT6) is a transcription factor that activates gene expression and is associated with the development of T-helper type 2 (Th2) cells and Th2 immune response. Modulating STAT6 activity, for example via inhibition and/or degradation, can lead to the treatment of a number of inflammatory conditions such as immune mediated diseases or conditions, IL-4/IL-13 mediated conditions, dermatology and allergic disease indications, and atopic dermatitis. Accordingly, there is a need for compounds, pharmaceutical compositions, and methods for modulation of STAT6 activity and treating associated conditions with said compounds, compositions, and methods.

### SUMMARY

In one embodiment, the present disclosure provides a compound of Formula J:

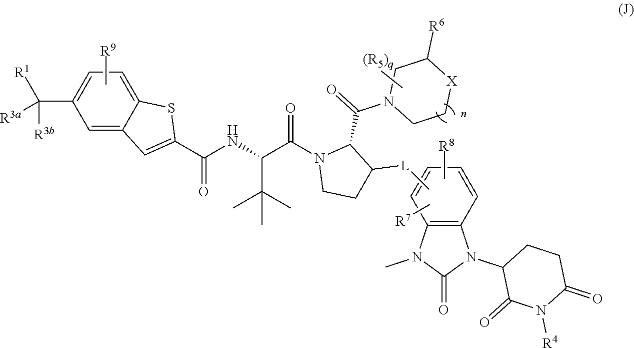

or a pharmaceutically acceptable salt thereof, wherein
R¹ is

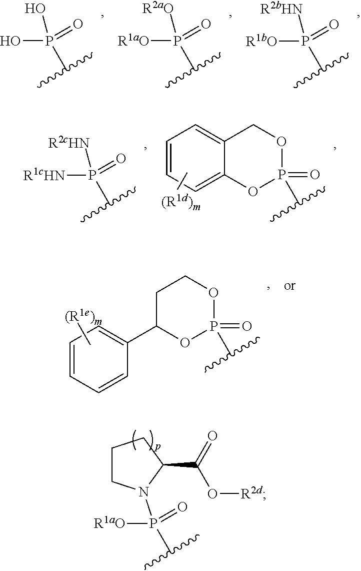

R^{1a} and R^{2a} are each independently C₁₋₆ alkyl, C₁₋₆ haloalkyl, C₁₋₆ alkylene-OC(O)—C₁₋₆ alkyl, C₁₋₆ alkylene-OC(O)O—C₁₋₆ alkyl, C₁₋₆ alkylene-SC(O)—C₁₋₆ alkyl, C₃₋₆ cycloalkyl, C₆₋₁₂ aryl, or

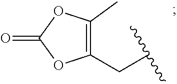 [SMILES: *Cc1oc(=O)oc1C]

R^{1b} is H, C₁₋₆ alkyl, C₁₋₆ haloalkyl, C₃₋₆ cycloalkyl or C₆₋₁₂ aryl;
R^{1c}, R^{2b}, and R^{2c} are each independently

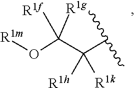

wherein
   R^{1f} and R^{1g} are each independently H or C₁₋₆ alkyl, or R^{1f} and R^{1g} together are oxo,
   R^{1h} and R^{1k}, are each independently H, C₁₋₆ alkyl, C₁₋₆ haloalkyl, C₂₋₆ alkoxyalkyl, or CH₂Ph, and
   R^{1m} is C₁₋₆ alkyl, C₁₋₆ haloalkyl, C₂₋₆ alkoxyalkyl, —C(O)—C₁₋₆ alkyl, —C(O)O—C₁₋₆ alkyl, C₃₋₈ cycloalkyl, —CH₂—C₃₋₈ cycloalkyl, heterocycloalkyl, or —CH₂- heterocycloalkyl, wherein each heterocycloalkyl has 3 to 8 ring members and 1 to 3 heteroatoms each independently N, O, or S, provided that when R^{1m} is —C(O)—C₁₋₆ alkyl or —C(O)O—C₁₋₆ alkyl, then R^{1f} and R^{1g} are each independently H or C₁₋₆ alkyl;
R^{1d} and R^{1e} are each independently C₁₋₆ alkyl, C₁₋₆ alkoxy, C₂₋₆ alkoxyalkyl, halogen, C₁₋₆ haloalkyl, C₁₋₆ haloalkoxy, or CN;
R^{2d} is C₁₋₆ alkyl or C₁₋₆ haloalkyl;
R^{3a} and R^{3b} are each independently H, C₁₋₆ alkyl, halogen, or C₁₋₆ haloalkyl, or R^{3a} and R^{3b} together are oxo;
L is -L¹-L²-L³-L⁴-L⁵-;
L¹ is absent, CH₂, O, or N(L^{1a});
L^{1a} is hydrogen or C₁₋₃ alkyl;
L² is absent, C₁₋₆ alkylene, C₁₋₆ alkylene-C(O), CH₂C(O)N(L^{2a}), CH₂C(O)N(L^{2a})-C₁₋₆ alkylene, —C(O)—, or —C(O)—C₁₋₆ alkylene, provided that when L² is —C(O)— or —C(O)—C₁₋₆ alkylene, then L¹ is absent, CH₂, or N(L^{1a});
L²a is hydrogen or C₁₋₆ alkyl;
L³ is absent, C₃₋₈ cycloalkylene, or a heterocycloalkylene having 4 to 11 ring members and 1 to 3 heteroatoms each independently N, O, or S, wherein each cycloalkylene and heterocycloalkylene is substituted with 0, 1, 2, or 3 R^{L3};
L⁴ is absent, C₁₋₆ alkylene, C₂₋₆ alkenylene, C₂₋₆ alkynylene, O, or C(O), wherein the alkylene is substituted with 0, 1, 2, or 3 R^{L4};
L⁵ is absent, C₁₋₆ alkylene, C₃₋₈ cycloalkylene, or a heterocycloalkylene having 4 to 11 ring members and 1 to 3 heteroatoms each independently N, O, or S, wherein each alkylene, cycloalkylene and heterocycloalkylene is substituted with 0, 1, 2, or 3 R^{L5}, and wherein no more than three of L¹, L², L³, L⁴, and L⁵ are absent;
each R^{L3}, R^{L4} and R^{L5} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
R⁴ is hydrogen, CH₂OC(O)—C₁₋₆ alkyl, CH₂OC(O)O—C₁₋₆ alkyl, or CH₂—OP(O)(OH)₂;
each R⁵ is independently halo, C₁₋₆ alkyl, or C₁₋₆ haloalkyl;
alternatively, where two R⁵ groups are bound to the same ring atom, such R⁵ groups can be taken together with the atom to which they are attached to form a C₃₋₅ cycloalkyl;
R⁶ is hydrogen, C₁₋₆ alkyl, C₁₋₆ alkoxy, C₆₋₁₀ aryl, heteroaryl having 5 to 10 ring members and 1 to 3 heteroatoms each independently N, O, or S, or —OR^{6a}, wherein the aryl and heteroaryl is substituted with 0, 1, 2, or 3 R^{6b};
R^{6a} is C₆₋₁₀ aryl or heteroaryl having 5 to 10 ring members and 1 to 3 heteroatoms each independently N, O, or S, wherein the aryl and heteroaryl is substituted with 0, 1, 2, or 3 R^{6c};
each R^{6b} and R^{6c} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
X is —CH₂—, —CF₂—, —CH(CN)—, —O—, or —N(R^{X})—;
R^{X} is hydrogen or C₁₋₆ alkyl;
subscript m is 0, 1, 2, or 3;
subscript n is 0, 1, or 2, provided that when subscript n is 0, then X is —CH₂— or —CH(CN)—, and if subscript n is 1 or 2 then X is —O—, —CH₂—, or —N(R^{X})—;
subscript p is 0, 1, or 2;
subscript q is 0, 1, or 2; and
R⁷, R⁸, and R⁹ are each independently hydrogen or fluorine.

In one embodiment, the present disclosure provides a compound of Formula I:

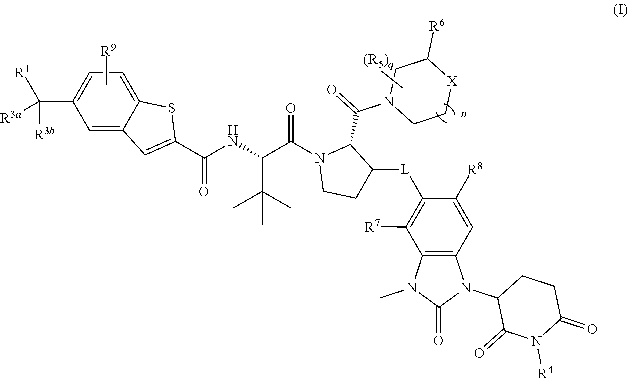

or a pharmaceutically acceptable salt thereof, wherein
R¹ is

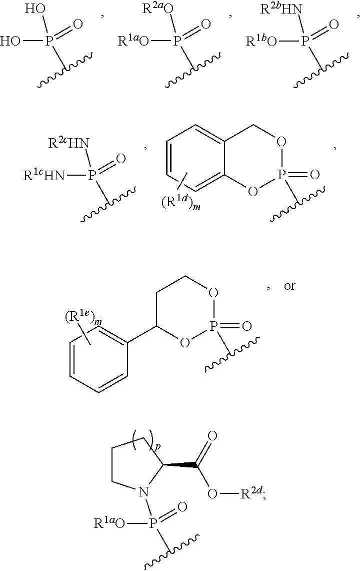

R^{1a} and R^{2a} are each independently C₁₋₆ alkyl, C₁₋₆ haloalkyl, C₁₋₆ alkylene-OC(O)—C₁₋₆ alkyl, C₁₋₆ alkylene-OC(O)O—C₁₋₆ alkyl, C₁₋₆ alkylene-SC(O)—C₁₋₆ alkyl, C₃₋₆ cycloalkyl, C₆₋₁₂ aryl, or

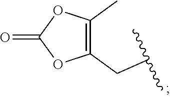 [SMILES: *Cc1oc(=O)oc1C]

R^{1b} is H, C₁₋₆ alkyl, C₁₋₆ haloalkyl, C₃₋₆ cycloalkyl or C₆₋₁₂ aryl;
R^{1c}, R^{2b}, and R^{2c} are each independently

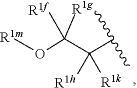

wherein
   R^{1f} and R^{1g} are each independently H or C₁₋₆ alkyl, or R^{1f} and R^{1g} together are oxo,
   R^{1h} and R^{1k}, are each independently H, C₁₋₆ alkyl, C₁₋₆haloalkyl, C₂₋₆ alkoxyalkyl, or CH₂Ph, and
   R^{1m} is C₁₋₆ alkyl, C₁₋₆ haloalkyl, C₂₋₆ alkoxyalkyl, —C(O)—C₁₋₆ alkyl, —C(O)O—C₁₋₆ alkyl, C₃₋₈ cycloalkyl, —CH₂—C₃₋₈ cycloalkyl, heterocycloalkyl, or —CH₂-heterocycloalkyl, wherein each heterocycloalkyl has 3 to 8 ring members and 1 to 3 heteroatoms each independently N, O, or S, provided that when R^{1m} is —C(O)—C₁₋₆ alkyl or —C(O)O—C₁₋₆ alkyl, then R^{1f} and R^{1g} are each independently H or C₁₋₆ alkyl;
R^{1d} and R^{1e} are each independently C₁₋₆ alkyl, C₁₋₆ alkoxy, C₂₋₆ alkoxyalkyl, halogen, C₁₋₆ haloalkyl, C₁₋₆ haloalkoxy, or CN;
R^{2d} is C₁₋₆ alkyl or C₁₋₆ haloalkyl;
R^{3a} and R^{3b} are each independently H, C₁₋₆ alkyl, halogen, or C₁₋₆ haloalkyl, or R^{3a} and R^{3b} together are oxo;
L is -L¹-L²-L³-L⁴-L⁵-;
L¹ is absent, CH₂, O, or N(L^{1a});
L^{1a} is hydrogen or C₁₋₃ alkyl;
L² is absent, C₁₋₆ alkylene, CH₂C(O), CH₂C(O)N(L^{2a}), CH₂C(O)N(L^{2a})-C₁₋₆ alkylene, —C(O)—, or —C(O)—C₁₋₆ alkylene, provided that when L² is —C(O)— or —C(O)—C₁₋₆ alkylene, then L¹ is absent, CH₂, or N(L^{1a});
L²a is hydrogen or C₁₋₆ alkyl;
L³ is absent, C₃₋₈ cycloalkylene, or a heterocycloalkylene having 4 to 11 ring members and 1 to 3 heteroatoms each independently N, O, or S, wherein each cycloalkylene and heterocycloalkylene is substituted with 0, 1, 2, or 3 R^{L3};
L⁴ is absent, C₁₋₆ alkylene, C₂₋₆ alkenylene, C₂₋₆ alkynylene, O, or C(O), wherein the alkylene is substituted with 0, 1, 2, or 3 R^{L4};
L⁵ is absent, C₁₋₆ alkylene, C₃₋₈ cycloalkylene, or a heterocycloalkylene having 4 to 11 ring members and 1 to 3 heteroatoms each independently N, O, or S, wherein each alkylene, cycloalkylene and heterocycloalkylene is substituted with 0, 1, 2, or 3 R^{L5}, and wherein no more than two of L¹, L², L³, L⁴, and L⁵ are absent;
each R^{L3}, R^{L4} and R^{L5} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
R⁴ is hydrogen, CH₂OC(O)—C₁₋₆ alkyl, CH₂OC(O)O—C₁₋₆ alkyl, or CH₂—OP(O)(OH)₂;
each R⁵ is independently C₁₋₆ alkyl, or C₁₋₆ haloalkyl;
alternatively, where two R⁵ groups are bound to the same ring atom, such R⁵ groups can be taken together with the atom to which they are attached to form a C₃₋₅ cycloalkyl;
R⁶ is hydrogen, C₁₋₆ alkyl, C₁₋₆ alkoxy, C₆₋₁₀ aryl, heteroaryl having 5 to 10 ring members and 1 to 3 heteroatoms each independently N, O, or S, or —OR^{6a}, wherein the aryl and heteroaryl is substituted with 0, 1, 2, or 3 R^{6b};
R^{6a} is C₆₋₁₀ aryl or heteroaryl having 5 to 10 ring members and 1 to 3 heteroatoms each independently N, O, or S, wherein the aryl and heteroaryl is substituted with 0, 1, 2, or 3 R^{6c};
each R^{6b} and R^{6c} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
X is —CH₂—, —CH(CN)—, —O—, or —N(R^{X})—;
RX is hydrogen or C₁₋₆ alkyl;
subscript m is 0, 1, 2, or 3;
subscript n is 0, 1, or 2, provided that when subscript n is 0, then X is —CH₂— or —CH(CN)—, and if subscript n is 1 or 2 then X is —O—, —CH₂—, or —N(R^{X})—; subscript p is 0, 1, or 2;
subscript q is 0, 1, or 2; and
R⁷, R⁸, and R⁹ are each independently hydrogen or fluorine.

In another embodiment, the present disclosure provides a pharmaceutical composition comprising a compound of the present disclosure, and a pharmaceutically acceptable excipient.

A method of treating an immune mediated disease or condition in a subject in need thereof, the method comprising administering to the subject a therapeutically effective amount of a compound of the present disclosure, or a pharmaceutically acceptable salt thereof, or a pharmaceutical composition of the present disclosure.

In another embodiment, the present disclosure provides a method of modulating a signal transducer and activator of transcription 6 (STAT6) protein activity in a subject in need thereof, the method comprising administering to the subject a therapeutically effective amount of a compound of the present disclosure, or a pharmaceutically acceptable salt thereof, or a pharmaceutical composition of the present disclosure.

In another embodiment, the present disclosure provides a method of treating a disease or condition mediated by a signal transducer and activator of transcription 6 (STAT6) protein in a subject in need thereof, the method comprising administering to the subject a therapeutically effective amount of a compound of the present disclosure, or a pharmaceutically acceptable salt thereof, or a pharmaceutical composition of the present disclosure.

In some embodiments, the present disclosure provides a method of modulating a signal transducer and activator of transcription 6 (STAT6) activity in a subject, wherein the method comprises administering to the subject a therapeutically effective amount of a compound of the present disclosure, or a pharmaceutically acceptable salt thereof, or a pharmaceutical composition of the present disclosure.

In some embodiments, the present disclosure provides a method of treating a disease or condition mediated by interleukin 4 (IL-4) or interleukin 3 (IL-3) in a subject in need thereof, the method comprising administering to the subject a therapeutically effective amount of a compound of the present disclosure, or a pharmaceutically acceptable salt thereof, or a pharmaceutical composition of the present disclosure.

In some embodiments, the present disclosure provides a method for manufacturing a medicament for treating a disease or condition mediated by a signal transducer and activator of transcription 6 (STAT6) protein in a subject in need thereof, characterized in that a compound of the present disclosure, or a pharmaceutically acceptable salt thereof, is used.

In some embodiments, the present disclosure provides use of the compound of the present disclosure, or a pharmaceutically acceptable salt thereof, for the manufacture of a medicament for the treatment of a disease or condition mediated by a signal transducer and activator of transcription 6 (STAT6) protein in a subject.

In some embodiments, the present disclosure provides the compound of the present disclosure, or a pharmaceutically acceptable salt thereof, for use in treating a disease or condition mediated by a signal transducer and activator of transcription 6 (STAT6) protein in a subject in need thereof.

In some embodiments, the present invention provides the compound of the present disclosure, or a pharmaceutically acceptable salt thereof, for use in therapy.

Also disclosed herein are compounds, and pharmaceutically acceptable salts thereof, of sub-formulas of Formulas I and Ia.

### DETAILED DESCRIPTION

### I. General

The disclosure relates generally to methods and compounds, and pharmaceutically acceptable salts thereof, for modulating a Signal transducer and activator of transcription 6 (STAT6) protein activity and treating STAT6 associated diseases. The following description sets forth exemplary methods, parameters and the like. It should be recognized, however, that such description is not intended as a limitation on the scope of the present disclosure but is instead provided as a description of exemplary embodiments.

### II. Definitions

As used in the present specification, the following words, phrases and symbols are generally intended to have the meanings as set forth below, except to the extent that the context in which they are used indicates otherwise.

Unless defined otherwise, all technical and scientific terms used herein have the same meaning as commonly understood by one of ordinary skill in the art. A dash at the front or end of a chemical group is a matter of convenience; chemical groups can be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both can be connected in either direction. A prefix such as “Cᵤ₋ᵥ”, “Cᵤ-Cᵥ” or “(Cᵤ-Cᵥ)” indicates that the following group has from u to v carbon atoms. For example, “C₁₋₆ alkyl” and “C₁-C₆ alkyl” both indicate that the alkyl group has from 1 to 6 carbon atoms.

Unless otherwise specified, the carbon atoms of the compounds of Formula J, I, or Ia, are intended to have a valence of four. If in some chemical structure representations, carbon atoms do not have a sufficient number of variables attached to produce a valence of four, the remaining carbon substituents needed to provide a valence of four should be assumed to be hydrogen.

A dash (“-”) that is not between two letters or symbols is used to indicate a point of attachment for a substituent. For example, —CONH₂ is attached through the carbon atom. A dash at the front or end of a chemical group is a matter of convenience; chemical groups can be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

A squiggly line on a chemical group as shown below, for example,

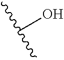 [SMILES: *O]

indicates a point of attachment, i.e., it shows the broken bond by which the group is connected to another described group.

As used herein, “a compound of the disclosure” or “a compound of the present disclosure” can mean a compound of any of the Formula I and Ia, or a pharmaceutically acceptable salt thereof.

Similarly, the phrase “a compound of Formula (number)” means a compound of that formula and pharmaceutically acceptable salts thereof.

The prefix “Cᵤ₋ᵥ” and “Cᵤ-Cᵥ” indicates that the following group has from u to v carbon atoms. For example, “C₁₋₈ alkyl” and “C₁-C₈ alkyl” indicates that the alkyl group has from 1 to 8 carbon atoms.

The term “adjacent carbons” and “adjacent atoms” as used herein refers to consecutive carbon atoms that are directly attached to each other. For example, in

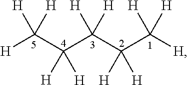

C₁ and C₂ are adjacent carbons, C₂ and C₃ are adjacent carbons, C₃ and C₄ are adjacent carbons, and C₄ and C₅ are adjacent carbons. Similarly, in

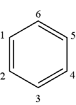

C₁ and C₂ are adjacent carbons, C₂ and C₃ are adjacent carbons, C₃ and C₄ are adjacent carbons, and C₄ and C₅ are adjacent carbons, C₅ and C₆ are adjacent carbons and C₆ and C₁ are adjacent carbons.

The term “double bond” as used herein refers to the formation of an additional single bond between two adjacent atoms that are already connected by a single bond, thus forming a double bond. For example, in

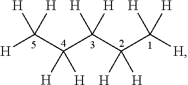

C₁ and C₂ are adjacent carbons, C₂ and C₃ are adjacent carbons, C₃ and C₄ are adjacent carbons, and C₄ and C₅ are adjacent carbons, such that two hydrogens, or R groups, on adjacent atoms are combined with the atoms to which they are attached and the bond linking the adjacent atoms to form a double bond as shown in the following:

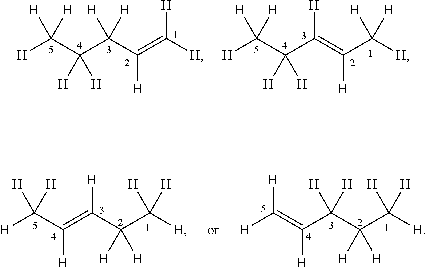

### Similarly, in

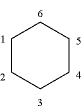

C₁ and C₂ are adjacent carbons, C₂ and C₃ are adjacent carbons, C₃ and C₄ are adjacent carbons, C₄ and C₅ are adjacent carbons, C₅ and C₆ are adjacent carbons, and C₆ and C₁ are adjacent carbons, such that two hydrogens, or R groups, on adjacent atoms are combined with the atoms to which they are attached and the bond linking the adjacent atoms to form a double bond as shown in the following:

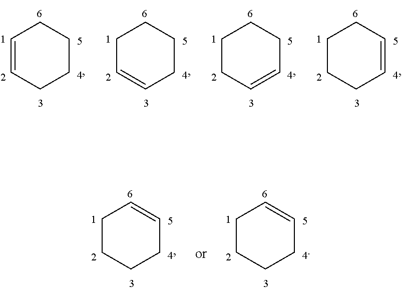 [SMILES: C1=CCCCC1 ; C1=CCCCC1 ; C1=CCCCC1 ; C1=CCCCC1 ; C1=CCCCC1 ; C1=CCCCC1]

The term “non-adjacent carbons” and “non-adjacent atoms” as used herein refers to nonconsecutive carbons atoms that are not directly attached to each other. For example, in

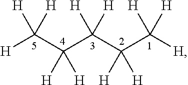

C₁ and C₃ are non-adjacent carbons, C₁ and C₄ are non-adjacent carbons, C₂ and C₄ are non-adjacent carbons, C₁ and C₅ are non-adjacent carbons, C₂ and C₅ are non-adjacent carbons, and C₃ and C₅ are non-adjacent carbons, among others. Similarly, in

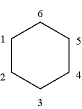

C₁ and C₃ are non-adjacent carbons, C₁ and C₄ are non-adjacent carbons, C₂ and C₄ are non-adjacent carbons, C₁ and C₅ are non-adjacent carbons, C₂ and C₅ are non-adjacent carbons, C₃ and C₅ are non-adjacent carbons, C₂ and C₆ are non-adjacent carbons, C₃ and C₆ are non-adjacent carbons, and C₄ and C₆ are non-adjacent carbons.

“Alkyl” refers to an unbranched or branched saturated hydrocarbon chain. For example, an alkyl group can have 1 to 20 carbon atoms (i.e., C₁-C₂₀ alkyl), 1 to 8 carbon atoms (i.e., C₁-C₈ alkyl), 1 to 6 carbon atoms (i.e., C₁-C₆ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. Other alkyl groups include, but are not limited to, heptyl, octyl, nonyl, decyl, undecyl, dodecyl, pentadcyl, hexadecyl, heptadecyl and octadecyl.

“Alkenyl” refers to an unbranched or branched hydrocarbon chain containing at least two carbon atoms and at least one carbon-carbon double bond. As used herein, alkenyl can have from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Alkenyl can include any number of carbons, such as C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, or any range therein. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

“Alkynyl” refers to an unbranched or branched hydrocarbon chain containing at least one carbon-carbon triple bond. For example, an alkynyl group can have from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond. Examples of C₂₋₆alkynyl include, but are not limited to, ethynyl, prop-1-ynyl, but-1-ynyl, pent-1-ynyl, pent-4-ynyl and penta-1,4-diynyl.

“Alkoxy” means a group having the formula —O-alkyl, in which an alkyl group, as defined above, is attached to the parent molecule via an oxygen atom. The alkyl portion of an alkoxy group can have 1 to 20 carbon atoms (i.e., C₁-C₂₀ alkoxy), 1 to 12 carbon atoms (i.e., C₁-C₁₂ alkoxy), 1 to 8 carbon atoms (i.e., C₁-C₈ alkoxy), 1 to 6 carbon atoms (i.e., C₁-C₆ alkoxy) or 1 to 3 carbon atoms (i.e., C₁-C₃ alkoxy). Examples of suitable alkoxy groups include, but are not limited to, methoxy (—O—CH₃ or —OMe), ethoxy (—OCH₂CH₃ or -OEt), isopropoxy (—O—CH(CH₃)₂), t-butoxy (—O—C(CH₃)₃ or -OtBu) and the like. Other examples of suitable alkoxy groups include, but are not limited to, sec-butoxy, tert-butoxy, pentoxy, hexoxy, and the like.

“Alkoxyalkyl” refers an alkoxy group linked to an alkyl group which is linked to the remainder of the compound. Alkoxyalkyl can have any suitable number of carbon, such as from 2 to 6 (C₂₋₆ alkoxyalkyl), 2 to 5 (C₂₋₅ alkoxyalkyl), 2 to 4 (C₂₋₄ alkoxyalkyl), or 2 to 3 (C₂₋₃ alkoxyalkyl). Alkoxy and alkyl are as defined above. Examples of “alkoxyalkyl” include, but are not limited to, methoxymethyl (CH₃OCH₂—), and methoxyethyl (CH₃OCH₂CH₂).

“Bridged” means a ring system in which non-adjacent atoms on a ring are connected by a divalent substituent, such as an alkylenyl or heteroalkylenyl group or a single heteroatom.

“Hydroxyalkyl” refers to a hydroxy group, —OH, linked to an alkyl group which is linked to the remainder of the compound such that the alkyl group is divalent. Hydroxyalkyl can have any suitable number of carbons, such as from 1 to 8 (C₁₋ₛ hydroxyalkyl), 1 to 6 (C₁₋₆ hydroxyalkyl), 2 to 6 (C₂₋₆ hydroxyalkyl), 2 to 4 (C₂₋₄ hydroxyalkyl), or 2 to 3 (C₂₋₃ hydroxyalkyl). Alkyl is as defined above where the alkyl is divalent.

“Halo” or “halogen” as used herein refers to fluoro (—F), chloro (—Cl), bromo (—Br) and iodo (—I).

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁₋₂₀ haloalkyl), 1 to 12 carbon atoms (i.e., C₁₋₁₂ haloalkyl), 1 to 8 carbon atoms (i.e., C₁₋₈ haloalkyl), 1 to 6 carbon atoms (i.e., C₁₋₆ haloalkyl) or 1 to 3 carbon atoms (i.e., C₁₋₃ haloalkyl). The alkyl groups can be substituted with 1, 2, 3, 4, 5, 6, 7, 8, 9 or more halogens. Examples of suitable haloalkyl groups include, but are not limited to, —CF₃, —CHF₂, —CFH₂, —CH₂CF₃, fluorochloromethyl, difluorochloromethyl, 1,1,1-trifluoroethyl and pentafluoroethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, 4, 5, 6, 7, 8, 9 or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Heteroalkyl” refers to an unbranched or branched saturated hydrocarbon chain containing from 1 to 4 heteroatoms.

“Cycloalkyl” refers to a saturated or partially saturated cyclic alkyl group having a single ring or multiple rings, such as 2, 3, 4 or more, wherein the multiple rings can be fused, bridged, spiro, or any combination thereof. As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include, but are not limited to, cyclopropyl, cyclobutyl, cyclopentyl, cyclohexyl, cycloheptyl, and cyclooctyl. Cycloalkyl groups also include partially unsaturated ring systems containing one or more double bonds, including fused ring systems with one aromatic ring and one non-aromatic ring, but not fully aromatic ring systems.

“Alkyl-cycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

The term “fused” refers to a ring system in which two or more rings in the system share a pair of adjacent ring atoms.

“Spiro” refers to at least two rings are linked together by one common atom. “Spiro” also refers to a ring substituent which is joined by two bonds at the same carbon atom. Examples of spiro groups include, but are not limited to, 1,1-diethylcyclopentane, dimethyl-dioxolane, and 4-benzyl-4-methylpiperidine, wherein the cyclopentane and piperidine, respectively, are the spiro substituents.

“Heterocycle” or “heterocyclyl” or “heterocycloalkyl” refer to a saturated or unsaturated cyclic alkyl group, with one or more ring heteroatoms independently selected from nitrogen, oxygen, sulfur and silicon. A heterocyclyl can be a single ring or multiple rings, such as 2, 3, 4 or more, wherein the multiple rings can be fused, bridged, spiro, or any combination thereof. As used herein, heterocyclyl has 3 to 20 ring atoms (i.e., 3 to 20 membered heterocyclyl), 3 to 12 ring atoms (i.e., 3 to 12 membered heterocyclyl), 3 to 10 ring atoms (i.e., 3 to 10 membered heterocyclyl), 3 to 8 ring atoms (i.e., 3 to 8 membered heterocyclyl), 4 to 12 ring carbon atoms (i.e., 4 to 12 membered heterocyclyl), 4 to 8 ring atoms (i.e., 4 to 8 membered heterocyclyl), or 4 to 6 ring atoms (i.e., 4 to 6 membered heterocyclyl). Examples of heterocyclyl groups include pyrrolidinyl, piperidinyl, tetrahydropyridinyl, piperazinyl, oxetanyl, dihydropyranyl, dioxolanyl, azetidinyl, and morpholinyl.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heterocycloalkyl component is as defined above.

“Aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 10 carbon atoms. Exemplary aryl groups include, but are not limited to, radicals derived from benzene (e.g., phenyl), naphthalene, anthracene, biphenyl, and the like.

“Alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above. Examples of alkyl-aryl groups include, but are not limited to, benzyl and ethyl-benzene.

“Heteroaryl” refers to an aromatic group, including groups having an aromatic tautomer or resonance structure, having a single ring, multiple rings, or multiple fused rings, with at least one heteroatom in the ring, i.e., one or more ring heteroatoms independently selected from nitrogen, oxygen, and sulfur, wherein the nitrogen or sulfur can be oxidized. Thus, the term includes rings having one or more annular O, N, S, S(O), S(O)₂, and N-oxide groups. The term includes rings having one or more annular C(O) groups. As used herein, heteroaryl include 5 to 20 ring atoms (i.e., 5- to 20-membered heteroaryl), 5 to 12 ring atoms (i.e., 5- to 12-membered heteroaryl), or 5 to 10 ring atoms (i.e., 5- to 10-membered heteroaryl), and 1 to 5 heteroatoms independently selected from nitrogen, oxygen, and sulfur, and oxidized forms of the heteroatoms. Examples of heteroaryl groups include, but are not limited to, pyridin-2(1H)-one, pyridazin-3(2H)-one, pyrimidin-4(3H)-one, quinolin-2(1H)-one, pyrimidinyl, purinyl, pyridyl, pyridazinyl, benzothiazolyl, and pyrazolyl. Heteroaryl does not encompass or overlap with aryl as defined above.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heteroaryl component is as defined within.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, formulations, dosage forms and other materials which are useful in preparing a pharmaceutical composition that is suitable for veterinary or human pharmaceutical use.

“Solvate” as used herein refers to the result of the interaction of a solvent and a compound. Solvates of salts of the compounds described herein are also provided. Hydrates of the compounds described herein are also provided.

“Prodrug” as used herein refers to a derivative of a drug that upon administration to the human body is converted to the parent drug according to some chemical or enzymatic pathway.

The compounds described herein can be prepared and/or formulated as pharmaceutically acceptable salts or when appropriate as a free base. Pharmaceutically acceptable salts are non-toxic salts of a free base form of a compound that possess the desired pharmacological activity of the free base. These salts can be derived from inorganic or organic acids or bases. For example, a compound that contains a basic nitrogen can be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in REMINGTON: THE SCIENCE AND PRACTICE OF PHARMACY, 21^{st} Edition, Lippincott Williams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

Provided are also compounds described herein or pharmaceutically acceptable salts, isomers, or a mixture thereof, in which from 1 to n hydrogen atoms attached to a carbon atom can be replaced by a deuterium atom or D, in which n is the number of hydrogen atoms in the molecule. As known in the art, the deuterium atom is a non-radioactive isotope of the hydrogen atom. Such compounds can increase resistance to metabolism, and thus can be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” TRENDS PHARMACOL. SCI., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formulas I and Ia, can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

“Racemates” refers to a mixture of enantiomers. The mixture can comprise equal or unequal amounts of each enantiomer.

“Stereoisomer” and “stereoisomers” refer to compounds that differ in the chirality of one or more stereocenters. Stereoisomers include enantiomers and diastereomers. The compounds can exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of ADVANCED ORGANIC CHEMISTRY, 4th ed., J. March, John Wiley & Sons, New York, 1992).

As used herein, “pharmaceutically acceptable carrier” or “pharmaceutically acceptable excipient” includes, but is not limited to, any and all solvents, dispersion media, coatings, antibacterial and antifungal agents, isotonic and absorption delaying agents, and combinations thereof. The use of pharmaceutically acceptable carriers and pharmaceutically acceptable excipients for pharmaceutically active substances is well known in the art. Except insofar as any conventional media or agent is incompatible with the active ingredient, its use in the therapeutic formulations is contemplated. Supplementary active ingredients can also be incorporated into the formulations. The carrier(s) must be “acceptable” in the sense of being compatible with the other ingredients of the formulation and physiologically innocuous to the recipient thereof.

“Biological sample” refers to, without limitation, cell cultures or extracts thereof; biopsied material obtained from a mammal or extracts thereof; and blood, saliva, urine, feces, semen, tears, or other body fluids or extracts thereof.

“STAT6 modulator” refers to compounds of the present disclosure, including compounds of Formula I and Ia, that inhibit or reduce some or all of the activity of the Signal transducer and activator of transcription 6 (STAT6), including STAT6 degraders.

“STAT6 degrader” refers to compounds of the present disclosure, including compounds of Formula I and Ia, that bind to and/or inhibit both STAT6 protein and an E3 ligase with measurable affinity, resulting in the ubiquitination and subsequent degradation of the STAT6 protein. In certain embodiments, a STAT6 degrader has an DC50 of less than about 50 pM, less than about 1 pM, less than about 500 nM. less than about 100 nM, less than about 10 nM, or less than about 1 nM. As used herein, the term “monovalent” refers to a degrader compound without an appended E3 ligase binding moiety.

“STAT6-mediated” disorders, diseases, and/or conditions refers to any disease or other deleterious condition in which STAT6 or a mutant thereof, are known to play a role.

“Disease” or “condition” refer to a state of being or health status of a patient or subject capable of being treated with a compound, pharmaceutical composition, or method provided herein. The disease may be an autoimmune, inflammatory, cancer, infectious (e.g., a viral infection), metabolic, developmental, cardiovascular, liver, intestinal, endocrine, neurological, or other disease. In some embodiments, the disease is an inflammatory condition such as immune mediated diseases or conditions, IL-4/IL-13 mediated conditions, dermatology and allergic disease indications, and atopic dermatitis.

A “subject” or “patient” is meant to describe a human or vertebrate animal including a dog, cat, horse, cow, mouse, or the like.

“Treatment” or “treating” is an approach for obtaining beneficial or desired results including clinical results. Beneficial or desired clinical results may include one or more of the following: (a) inhibiting the disease or condition (e.g., decreasing one or more symptoms resulting from the disease or condition, and/or diminishing the extent of the disease or condition); (b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

The term “therapeutically effective amount,” as used herein, is the amount of compound disclosed herein present in a formulation described herein that is needed to provide a desired level of drug in the secretions and tissues of the airways and lungs, or alternatively, in the bloodstream of a subject to be treated to give an anticipated physiological response or desired biological effect when such a formulation is administered by the chosen route of administration. The precise amount will depend upon numerous factors, for example the particular compound disclosed herein, the specific activity of the formulation, the delivery device employed, the physical characteristics of the formulation, its intended use, as well as subject considerations such as severity of the disease state, subject cooperation, etc., and can readily be determined by one skilled in the art based upon the information provided herein.

“Administering” refers to oral administration, administration as a suppository, topical contact (e.g., transdermal), parenteral, intravenous, intraperitoneal, intramuscular, intralesional, intranasal, inhaled, intradermal, and/or subcutaneous administration, intrathecal administration, and/or the implantation of a slow-release device e.g., a mini-osmotic pump, to the subject. The administration can be carried out according to a schedule specifying frequency of administration, dose for administration, and other factors.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

### III. Compounds

Disclosed herein are, among other things, compounds of Formula J, I, and Ia.

In some embodiments, the present disclosure provides a compound of Formula J:

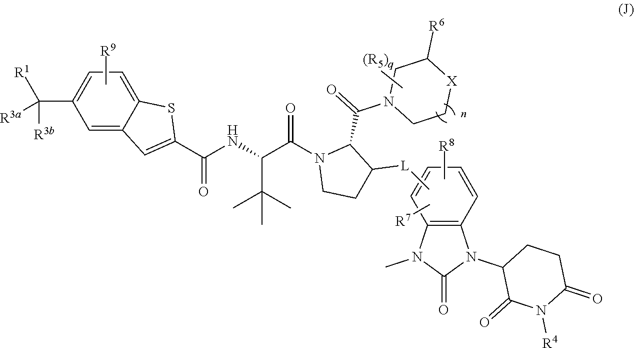

or a pharmaceutically acceptable salt thereof, wherein
R¹ is

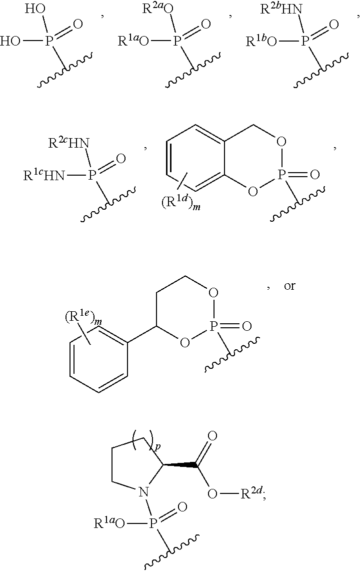

R^{1a} and R^{2a} are each independently C₁₋₆ alkyl, C₁₋₆ haloalkyl, C₁₋₆ alkylene-OC(O)—C₁₋₆ alkyl, C₁₋₆ alkylene-OC(O)O—C₁₋₆ alkyl, C₁₋₆ alkylene-SC(O)—C₁₋₆ alkyl, C₃₋₆ cycloalkyl, C₆₋₁₂ aryl, or

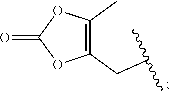 [SMILES: *Cc1oc(=O)oc1C]

R^{1b} is H, C₁₋₆ alkyl, C₁₋₆ haloalkyl, C₃₋₆ cycloalkyl or C₆₋₁₂ aryl;
R^{1c}, R^{2b}, and R^{2c} are each independently

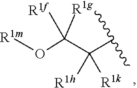

wherein
   R^{1f} and R^{1g} are each independently H or C₁₋₆ alkyl, or R^{1f} and R^{1g} together are oxo,
   R^{1h} and R^{1k}, are each independently H, C₁₋₆ alkyl, C₁₋₆ haloalkyl, C₂₋₆ alkoxyalkyl, or CH₂Ph, and
   R^{1m} is C₁₋₆ alkyl, C₁₋₆ haloalkyl, C₂₋₆ alkoxyalkyl, —C(O)—C₁₋₆ alkyl, —C(O)O—C₁₋₆ alkyl, C₃₋₈ cycloalkyl, —CH₂—C₃₋₈ cycloalkyl, heterocycloalkyl, or —CH₂-heterocycloalkyl, wherein each heterocycloalkyl has 3 to 8 ring members and 1 to 3 heteroatoms each independently N, O, or S, provided that when R^{1m} is —C(O)—C₁₋₆ alkyl or —C(O)O—C₁₋₆ alkyl, then R^{1f} and R^{1g} are each independently H or C₁₋₆ alkyl;
R^{1d} and R^{1e} are each independently C₁₋₆ alkyl, C₁₋₆ alkoxy, C₂₋₆ alkoxyalkyl, halogen, C₁₋₆ haloalkyl, C₁₋₆ haloalkoxy, or CN;
R^{2d} is C₁₋₆ alkyl or C₁₋₆ haloalkyl;
R^{3a} and R^{3b} are each independently H, C₁₋₆ alkyl, halogen, or C₁₋₆ haloalkyl, or R^{3a} and R^{3b} together are oxo;
L is -L¹-L²-L³-L⁴-L⁵-;
L¹ is absent, CH₂, O, or N(L^{1a});
L^{1a} is hydrogen or C₁₋₃ alkyl;
L² is absent, C₁₋₆ alkylene, C₁₋₆ alkylene-C(O), CH₂C(O)N(L^{2a}), CH₂C(O)N(L^{2a})-C₁₋₆ alkylene, —C(O)—, or —C(O)—C₁₋₆ alkylene, provided that when L² is —C(O)— or —C(O)—C₁₋₆ alkylene, then L¹ is absent, CH₂, or N(L^{1a});
L²a is hydrogen or C₁₋₆ alkyl;
L³ is absent, C₃₋₈ cycloalkylene, or a heterocycloalkylene having 4 to 11 ring members and 1 to 3 heteroatoms each independently N, O, or S, wherein each cycloalkylene and heterocycloalkylene is substituted with 0, 1, 2, or 3 R^{L3};
L⁴ is absent, C₁₋₆ alkylene, C₂₋₆ alkenylene, C₂₋₆ alkynylene, O, or C(O), wherein the alkylene is substituted with 0, 1, 2, or 3 R^{L4};
L⁵ is absent, C₁₋₆ alkylene, C₃₋₈ cycloalkylene, or a heterocycloalkylene having 4 to 11 ring members and 1 to 3 heteroatoms each independently N, O, or S, wherein each alkylene, cycloalkylene and heterocycloalkylene is substituted with 0, 1, 2, or 3 R^{L5}, and wherein no more than three of L¹, L², L³, L⁴, and L⁵ are absent;
each R^{L3}, R^{L4} and R^{L5} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
R⁴ is hydrogen, CH₂OC(O)—C₁₋₆ alkyl, CH₂OC(O)O—C₁₋₆ alkyl, or CH₂—OP(O)(OH)₂;
each R⁵ is independently halo, C₁₋₆ alkyl, or C₁₋₆ haloalkyl;
alternatively, where two R⁵ groups are bound to the same ring atom, such R⁵ groups can be taken together with the atom to which they are attached to form a C₃₋₅ cycloalkyl;
R⁶ is hydrogen, C₁₋₆ alkyl, C₁₋₆ alkoxy, C₆₋₁₀ aryl, heteroaryl having 5 to 10 ring members and 1 to 3 heteroatoms each independently N, O, or S, or —OR^{6a}, wherein the aryl and heteroaryl is substituted with 0, 1, 2, or 3 R^{6b};
R^{6a} is C₆₋₁₀ aryl or heteroaryl having 5 to 10 ring members and 1 to 3 heteroatoms each independently N, O, or S, wherein the aryl and heteroaryl is substituted with 0, 1, 2, or 3 R^{6c};
each R^{6b} and R^{6c} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
X is —CH₂—, —CF₂—, —CH(CN)—, —O—, or —N(R^{X})—;
RX is hydrogen or C₁₋₆ alkyl;
subscript m is 0, 1, 2, or 3;
subscript n is 0, 1, or 2, provided that when subscript n is 0, then X is —CH₂— or —CH(CN)—, and if subscript n is 1 or 2 then X is —O—, —CH₂—, or —N(RX)—;
subscript p is 0, 1, or 2;
subscript q is 0, 1, or 2; and
R⁷, R⁸, and R⁹ are each independently hydrogen or fluorine.

In some embodiments, the present disclosure provides a compound of Formula I:

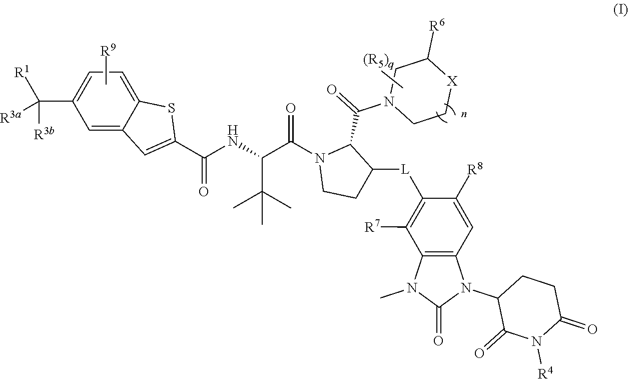

or a pharmaceutically acceptable salt thereof, wherein
R¹ is

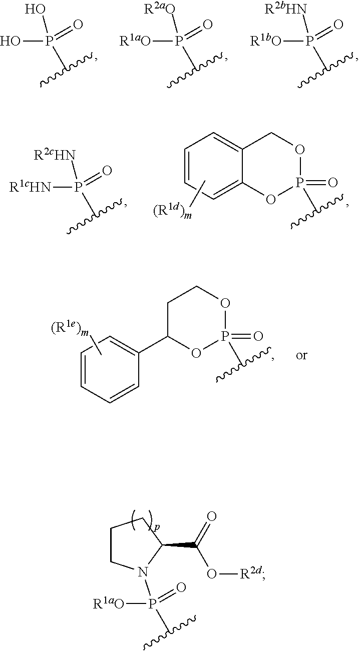

R^{1a} and R^{2a} are each independently C₁₋₆ alkyl, C₁₋₆ haloalkyl, C₁₋₆ alkylene-OC(O)—C₁₋₆ alkyl, C₁₋₆ alkylene-OC(O)O—C₁₋₆ alkyl, C₁₋₆ alkylene-SC(O)—C₁₋₆ alkyl, C₃₋₆ cycloalkyl, C₆₋₁₂ aryl, or

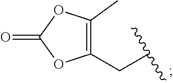 [SMILES: *Cc1oc(=O)oc1C]

R^{1b} is H, C₁₋₆ alkyl, C₁₋₆ haloalkyl, C₃₋₆ cycloalkyl or C₆₋₁₂ aryl;
R^{1c}, R^{2b}, and R^{2c} are each independently

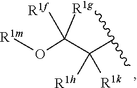

wherein
   R^{1f} and R^{1g} are each independently H or C₁₋₆ alkyl, or R^{1f} and R^{1g} together are oxo,
   R^{1h} and R^{1k}, are each independently H, C₁₋₆ alkyl, C₁₋₆ haloalkyl, C₂₋₆ alkoxyalkyl, or CH₂Ph, and
   R^{1m} is C₁₋₆ alkyl, C₁₋₆ haloalkyl, C₂₋₆ alkoxyalkyl, —C(O)—C₁₋₆ alkyl, —C(O)O—C₁₋₆ alkyl, C₃₋₈ cycloalkyl, —CH₂—C₃₋₈ cycloalkyl, heterocycloalkyl, or —CH₂-heterocycloalkyl, wherein each heterocycloalkyl has 3 to 8 ring members and 1 to 3 heteroatoms each independently N, O, or S, provided that when R¹ is —C(O)—C₁₋₆ alkyl or —C(O)O—C₁₋₆ alkyl, then R^{1f} and Rig are each independently H or C₁₋₆ alkyl;
R^{1d} and R^{1e} are each independently C₁₋₆ alkyl, C₁₋₆ alkoxy, C₂₋₆ alkoxyalkyl, halogen, C₁₋₆ haloalkyl, C₁₋₆ haloalkoxy, or CN;
R^{2d} is C₁₋₆ alkyl or C₁₋₆ haloalkyl;
R^{3a} and R^{3b} are each independently H, C₁₋₆ alkyl, halogen, or C₁₋₆ haloalkyl, or R^{3a} and R^{3b} together are oxo;
L is -L¹-L²-L³-L⁴-L⁵-;
L¹ is absent, CH₂, O, or N(L^{1a});
L^{1a} is hydrogen or C₁₋₃ alkyl;
L² is absent, C₁₋₆ alkylene, CH₂C(O), CH₂C(O)N(L^{2a}), CH₂C(O)N(L^{2a})-C₁₋₆ alkylene, —C(O)—, or —C(O)—C₁₋₆ alkylene, provided that when L² is —C(O)— or —C(O)—C₁₋₆ alkylene, then L¹ is absent, CH₂, or N(L^{1a});
L²a is hydrogen or C₁₋₆ alkyl;
L³ is absent, C₃₋₈ cycloalkylene, or a heterocycloalkylene having 4 to 11 ring members and 1 to 3 heteroatoms each independently N, O, or S, wherein each cycloalkylene and heterocycloalkylene is substituted with 0, 1, 2, or 3 R^{L3};
L⁴ is absent, C₁₋₆ alkylene, C₂₋₆ alkenylene, C₂₋₆ alkynylene, O, or C(O), wherein the alkylene is substituted with 0, 1, 2, or 3 R^{L4};
L⁵ is absent, C₁₋₆ alkylene, C₃₋₈ cycloalkylene, or a heterocycloalkylene having 4 to 11 ring members and 1 to 3 heteroatoms each independently N, O, or S, wherein each alkylene, cycloalkylene and heterocycloalkylene is substituted with 0, 1, 2, or 3 R^{L5}, and wherein no more than two of L¹, L², L³, L⁴, and L⁵ are absent;
each R^{L3}, R^{L4} and R^{L5} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
R⁴ is hydrogen, CH₂OC(O)—C₁₋₆ alkyl, CH₂OC(O)O—C₁₋₆ alkyl, or CH₂—OP(O)(OH)₂;
each R⁵ is independently C₁₋₆ alkyl, or C₁₋₆ haloalkyl;
alternatively, where two R⁵ groups are bound to the same ring atom, such R⁵ groups can be taken together with the atom to which they are attached to form a C₃₋₅ cycloalkyl;
R⁶ is hydrogen, C₁₋₆ alkyl, C₁₋₆ alkoxy, C₆₋₁₀ aryl, heteroaryl having 5 to 10 ring members and 1 to 3 heteroatoms each independently N, O, or S, or —OR^{6a}, wherein the aryl and heteroaryl is substituted with 0, 1, 2, or 3 R^{6b};
R^{6a} is C₆₋₁₀ aryl or heteroaryl having 5 to 10 ring members and 1 to 3 heteroatoms each independently N, O, or S, wherein the aryl and heteroaryl is substituted with 0, 1, 2, or 3 R^{6c};
each R^{6b} and R^{6c} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
X is —CH₂—, —CH(CN)—, —O—, or —N(R^{X})—;
RX is hydrogen or C₁₋₆ alkyl;
subscript m is 0, 1, 2, or 3;
subscript n is 0, 1, or 2, provided that when subscript n is 0, then X is —CH₂— or —CH(CN)—, and if subscript n is 1 or 2 then X is —O—, —CH₂—, or —N(R^{X})—;
subscript p is 0, 1, or 2;
subscript q is 0, 1, or 2; and
R⁷, R⁸, and R⁹ are each independently hydrogen or fluorine.

In some embodiments, the present disclosure provides a compound of Formula J or I, or a pharmaceutically acceptable salt thereof, having the structure of Formula Ia:

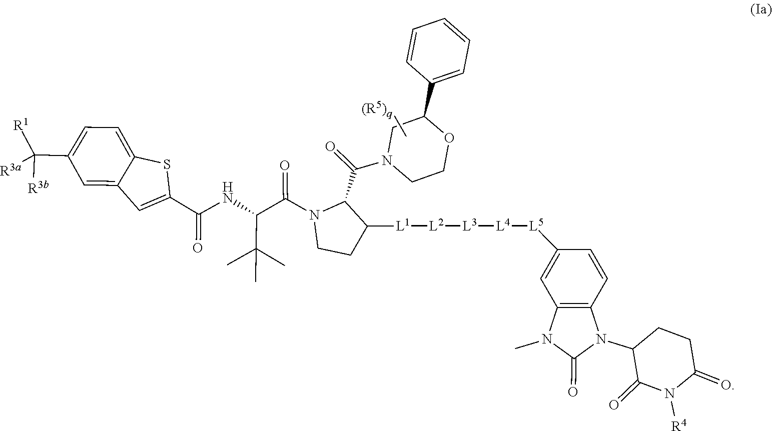

In some embodiments, the present disclosure provides a compound of Formula J, I, or Ia, or a pharmaceutically acceptable salt thereof, wherein R¹ is

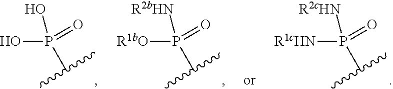

In some embodiments, the present disclosure provides a compound of Formula J, I, or Ia, or a pharmaceutically acceptable salt thereof, wherein R¹ is

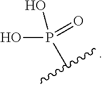 [SMILES: *P(=O)(O)O]

In some embodiments, the present disclosure provides a compound of Formula J, I, or Ia, or a pharmaceutically acceptable salt thereof, wherein R¹ is

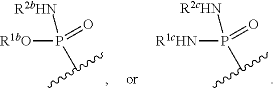

In some embodiments, the present disclosure provides a compound of Formula J, I, or Ia, or a pharmaceutically acceptable salt thereof, wherein
R^{1b} is C₆₋₁₂ aryl; and
R^{1c}, R^{2b}, and R^{2c} are each independently

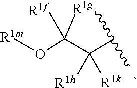

wherein
R^{1f} and R^{1g} together are oxo,
R^{1h} and R^{1k}, are each independently H or C₁₋₆ alkyl, and
R^{1m} is C₁₋₆ alkyl.

In some embodiments, the present disclosure provides a compound of Formula J, I, or Ia, or a pharmaceutically acceptable salt thereof, wherein R¹ is

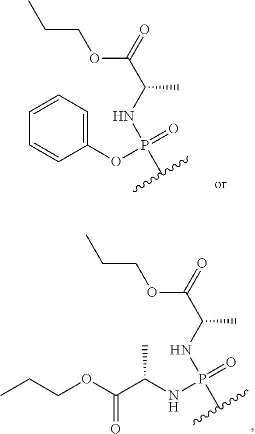

In some embodiments, the present disclosure provides a compound of Formula J, I, or Ia, or a pharmaceutically acceptable salt thereof, wherein
R^{3a} is hydrogen or F; and
R^{3b} is F.

In some embodiments, the present disclosure provides a compound of Formula J, I, or Ia, or a pharmaceutically acceptable salt thereof, wherein R^{3a} and R^{3b} are each fluoro.

In some embodiments, the present disclosure provides a compound of Formula J, I, or Ia, or a pharmaceutically acceptable salt thereof, wherein L¹ is absent, O, or NH. In some embodiments, the present disclosure provides a compound of Formula J, I, or Ia, or a pharmaceutically acceptable salt thereof, wherein L¹ is absent or O. In some embodiments, the present disclosure provides a compound of Formula J, I, or Ia, or a pharmaceutically acceptable salt thereof, wherein L¹ is O.

In some embodiments, the present disclosure provides a compound of Formula J, I, or Ia, or a pharmaceutically acceptable salt thereof, wherein L² is absent, CH₂, (CH₂)₂, (CH₂)₅, CH₂C(O), CH₂C(O)NHCH₂ or CH₂C(O)N(CH₃)CH₂. In some embodiments, the present disclosure provides a compound of Formula J, I, or Ia, or a pharmaceutically acceptable salt thereof, wherein L² is CH₂, (CH₂)₅, CH₂C(O), CH₂C(O)NHCH₂ or CH₂C(O)N(CH₃)CH₂. In some embodiments, the present disclosure provides a compound of Formula J, I, or Ia, or a pharmaceutically acceptable salt thereof, wherein L² is CH₂ or CH₂C(O). In some embodiments, the present disclosure provides a compound of Formula J, I, or Ia, or a pharmaceutically acceptable salt thereof, wherein L² is CH₂C(O).

In some embodiments, the present disclosure provides a compound of Formula J, I, or Ia, or a pharmaceutically acceptable salt thereof, wherein L³ is absent,

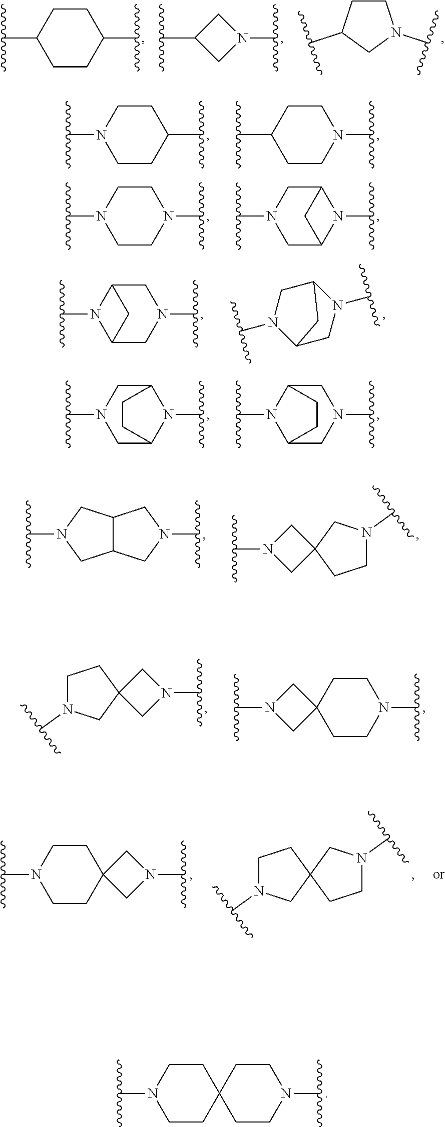

In some embodiments, the present disclosure provides a compound of Formula J, I, or Ia, or a pharmaceutically acceptable salt thereof, wherein L³ is absent,

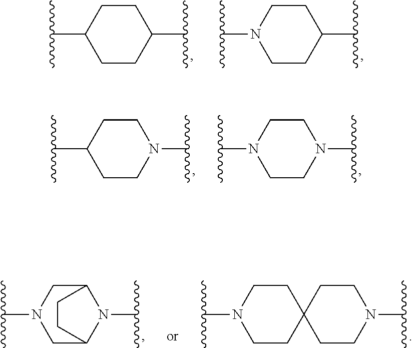

In some embodiments, the present disclosure provides a compound of Formula J, I, or Ia, or a pharmaceutically acceptable salt thereof, wherein L³ is

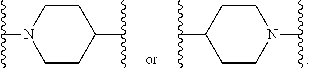 [SMILES: *C1CCN(*)CC1 ; *C1CCN(*)CC1]

In some embodiments, the present disclosure provides a compound of Formula J, I, or Ia, or a pharmaceutically acceptable salt thereof, wherein L⁴ is absent, CH₂, CH₂CH₂, —C≡C—, O, or C(O). In some embodiments, the present disclosure provides a compound of Formula J, I, or Ia, or a pharmaceutically acceptable salt thereof, wherein L⁴ is CH₂.

In some embodiments, the present disclosure provides a compound of Formula J, I, or Ia, or a pharmaceutically acceptable salt thereof, wherein L⁵ is absent,

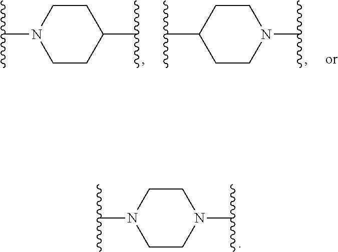

In some embodiments, the present disclosure provides a compound of Formula J, I, or Ia, or a pharmaceutically acceptable salt thereof, wherein L⁵ is absent,

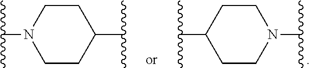 [SMILES: *C1CCN(*)CC1 ; *C1CCN(*)CC1]

In some embodiments, the present disclosure provides a compound of Formula J, I, or Ia, or a pharmaceutically acceptable salt thereof, wherein L⁵ is absent.

In some embodiments, the present disclosure provides a compound of Formula J, I, or Ia, or a pharmaceutically acceptable salt thereof, wherein at least two of L¹, L³ and L⁴ are not absent.

In some embodiments, the present disclosure provides a compound of Formula J, I, or Ia, or a pharmaceutically acceptable salt thereof, wherein L is:

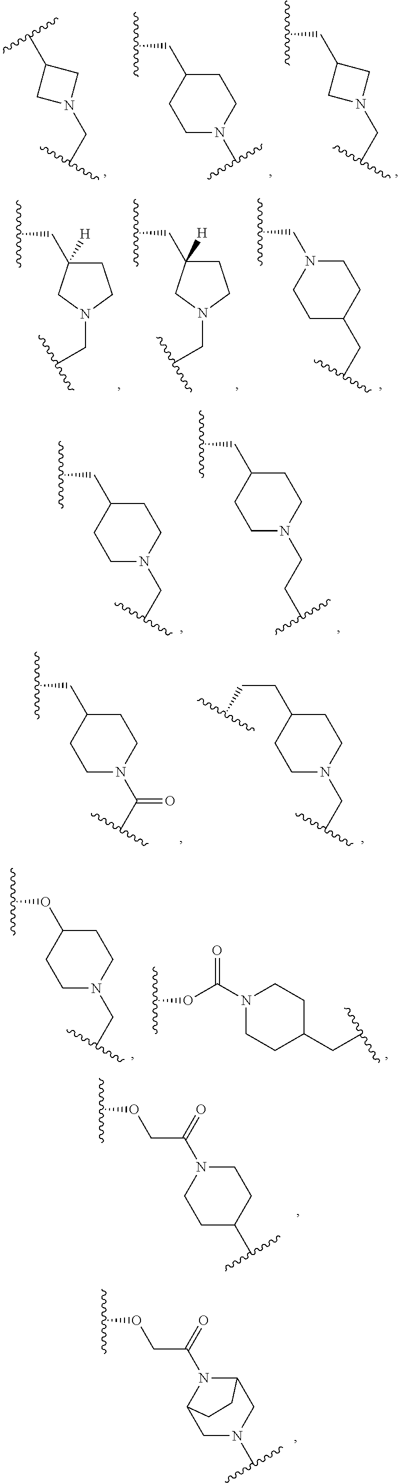

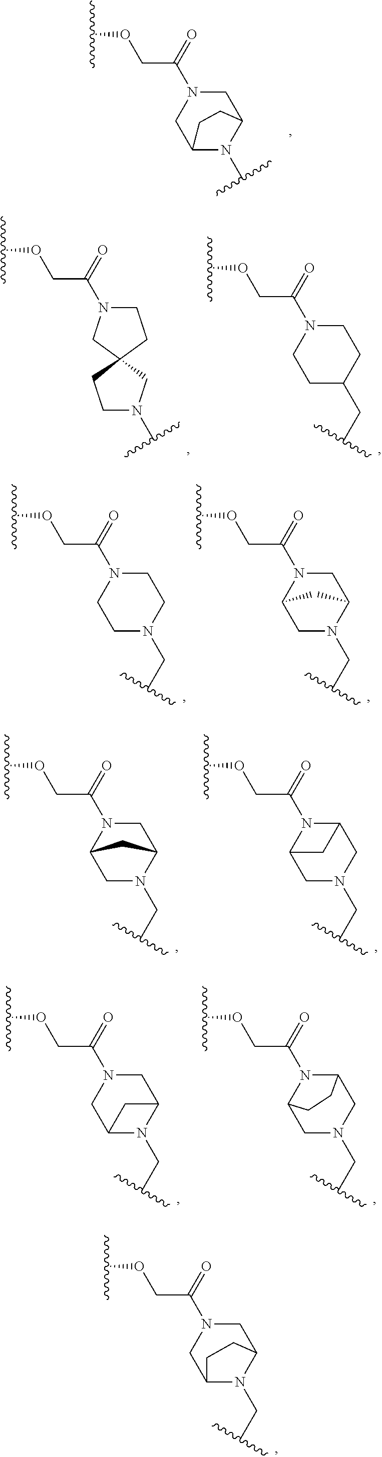

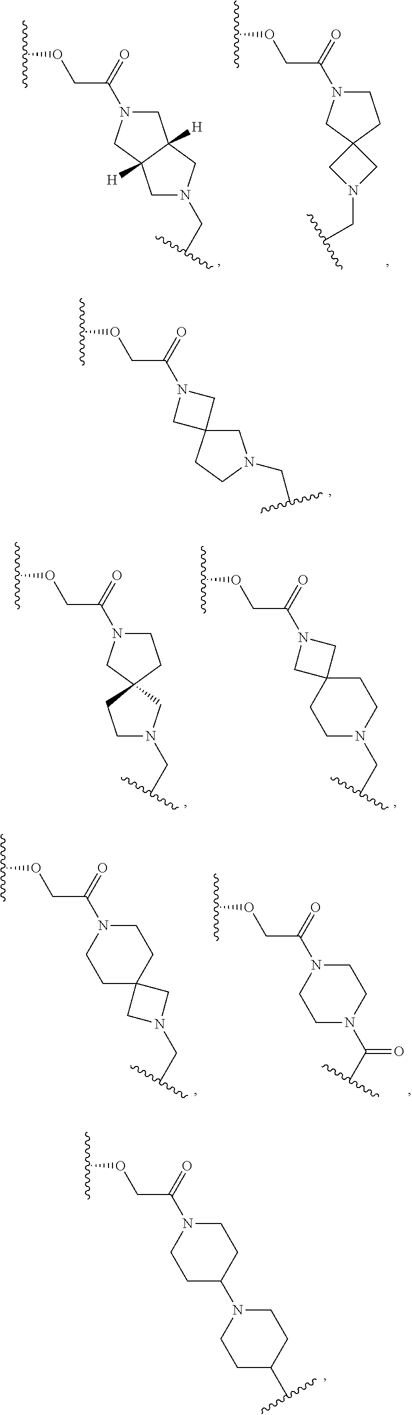

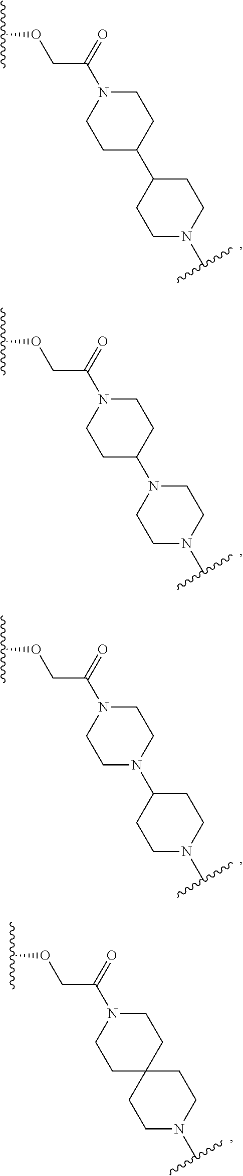

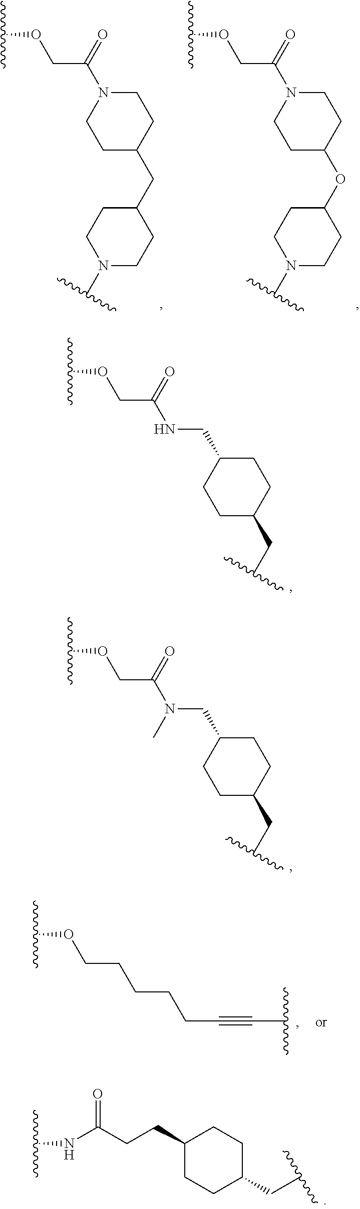

In some embodiments, the present disclosure provides a compound of Formula J, I, or Ia, or a pharmaceutically acceptable salt thereof, wherein L is:

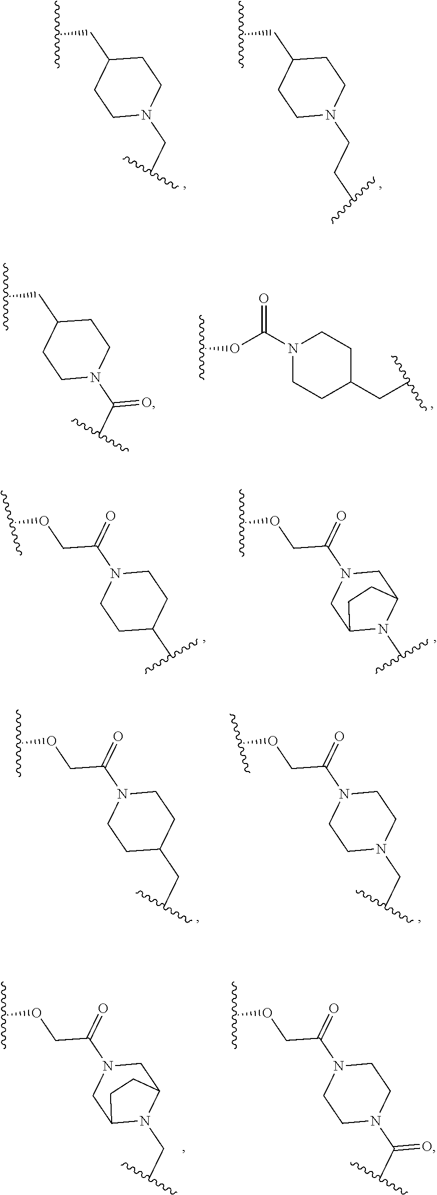

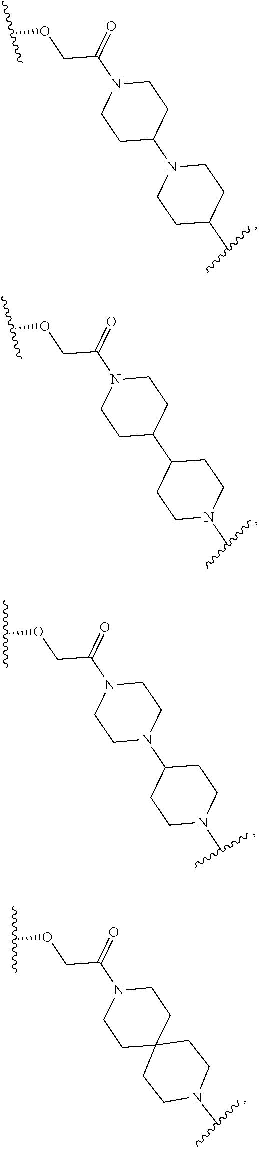

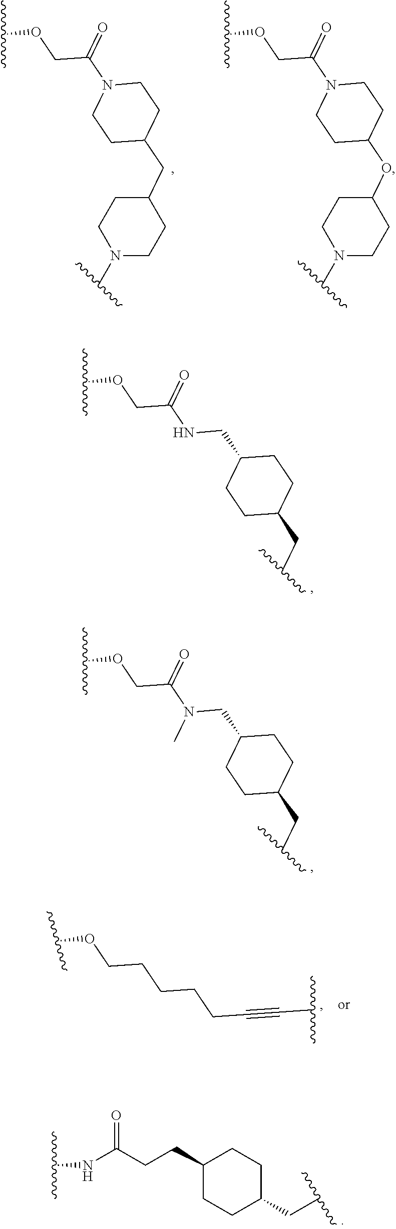

In some embodiments, the present disclosure provides a compound of Formula J, I, or Ia, or a pharmaceutically acceptable salt thereof, wherein L is:

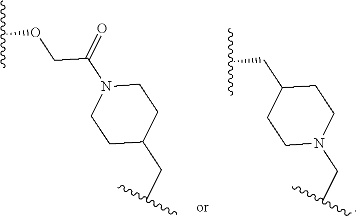

In some embodiments, the present disclosure provides a compound of Formula J, I, or Ia, or a pharmaceutically acceptable salt thereof, wherein R⁴ is hydrogen.

In some embodiments, the present disclosure provides a compound of Formula J, I, or Ia, or a pharmaceutically acceptable salt thereof, wherein
each R⁵ is hydrogen, or each R⁵ is F;
alternatively, where two R⁵ groups are bound to the same ring atom, such R⁵ groups can be taken together with the atom to which they are attached to form a C₃₋₅ cycloalkyl;
R⁶ is phenyl or pyridyl;
X is —CH₂—, —CH(CN)—, —CF₂—, —O—, or —N(CH₃)—;
subscript n is 0 or 1, provided that when subscript n is 0, then X is —CH₂— or —CH(CN)—, and if subscript n is 1 then X is —CH₂—, —CF₂—, —O—, or —N(CH₃)—; and
subscript q is 0 or 2.

In some embodiments, the present disclosure provides a compound of Formula J, I, or Ia, or a pharmaceutically acceptable salt thereof, wherein
each R⁵ is hydrogen;
alternatively, where two R⁵ groups are bound to the same ring atom, such R⁵ groups can be taken together with the atom to which they are attached to form a C₃₋₅ cycloalkyl;
R⁶ is phenyl or pyridyl;
X is —CH₂— or —O—;
subscript n is 0 or 1, provided that when subscript n is 0, then X is —CH₂—, and if subscript n is 1 then X is —O— or —CH₂—; and
subscript q is 0 or 2.

In some embodiments, the present disclosure provides a compound of Formula I or J, or a pharmaceutically acceptable salt thereof, wherein the moiety

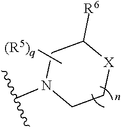

has the structure

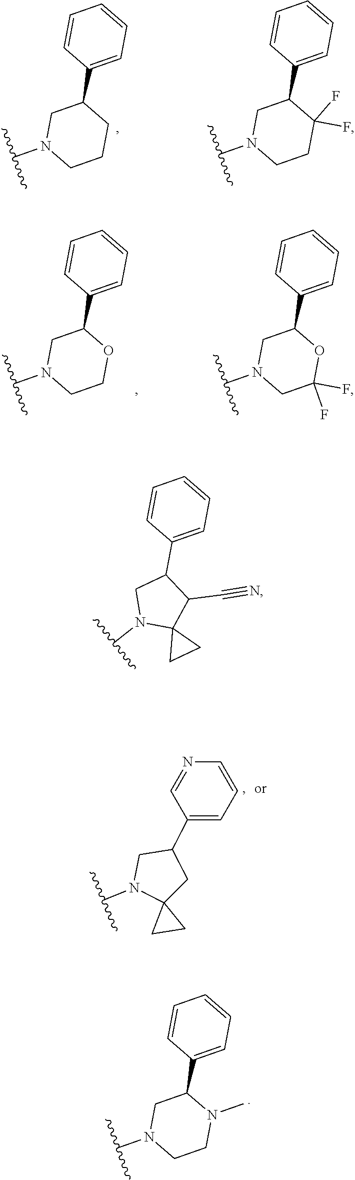

In some embodiments, the present disclosure provides a compound of Formula J or I, or a pharmaceutically acceptable salt thereof, wherein the moiety

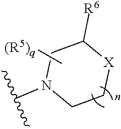

has the structure

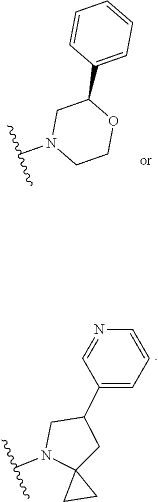 [SMILES: *N1CCO[C@H](c2ccccc2)C1 ; *N1CC(c2cccnc2)CC12CC2]

In some embodiments, the present disclosure provides a compound of Formula J or I, or a pharmaceutically acceptable salt thereof, wherein the moiety

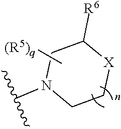

has the structure

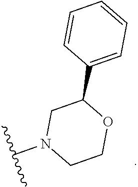 [SMILES: *N1CCO[C@H](c2ccccc2)C1]

In some embodiments, the present disclosure provides a compound of Formula J or I, or a pharmaceutically acceptable salt thereof, wherein R⁷, R⁸, and R⁹ are each hydrogen or F. In some embodiments, the present disclosure provides a compound of Formula J or I, or a pharmaceutically acceptable salt thereof, wherein R⁷, R⁸, and R⁹ are each hydrogen.

In some embodiments, the present disclosure provides a compound of Formula J, I, or Ia, or a pharmaceutically acceptable salt thereof, having the structure of:

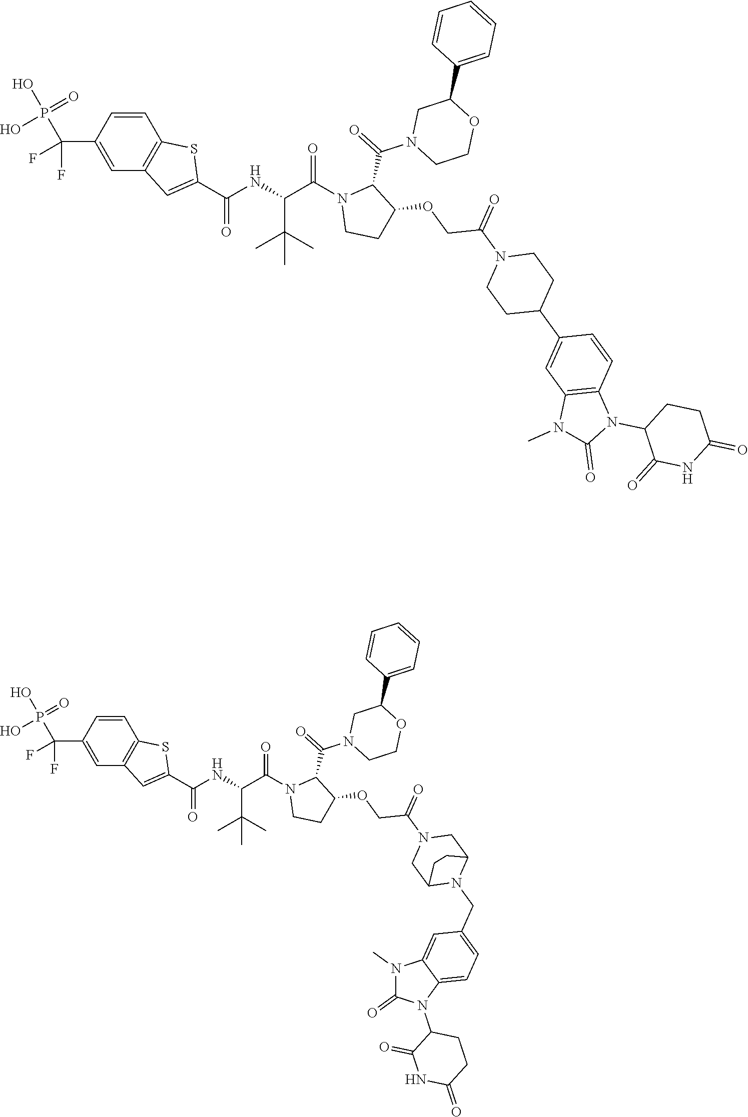 [SMILES: Cn1c(=O)n(C2CCC(=O)NC2=O)c2ccc(C3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(CN3C4CCC3CN(C(=O)CO[C@@H]3CCN(C(=O)[C@@H](NC(=O)c5cc6cc(C(F)(F)P(=O)(O)O)ccc6s5)C(C)(C)C)[C@@H]3C(=O)N3CCO[C@H](c5ccccc5)C3)C4)cc21]

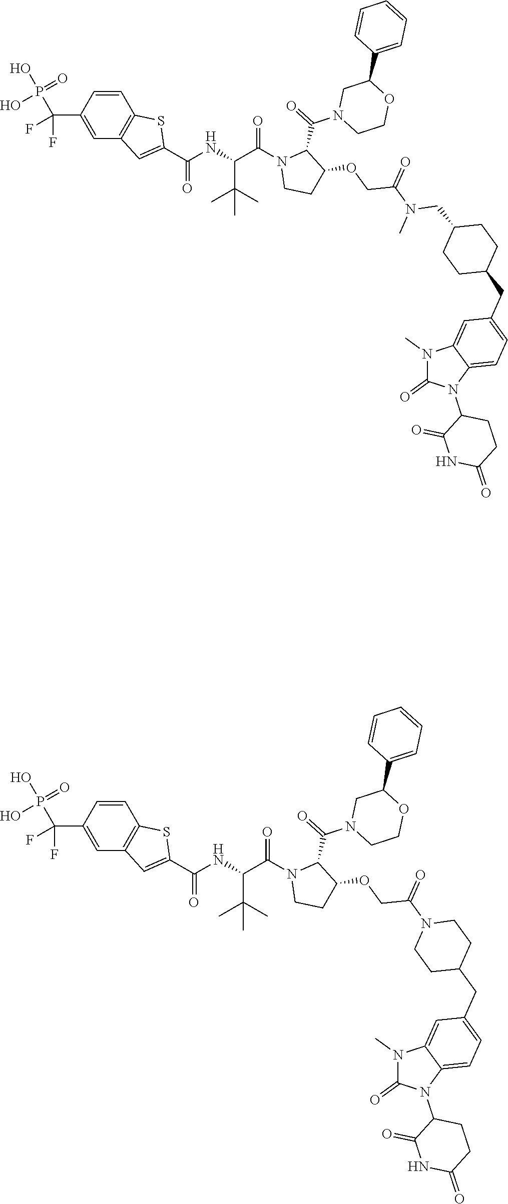 [SMILES: CN(C[C@H]1CC[C@H](Cc2ccc3c(c2)n(C)c(=O)n3C2CCC(=O)NC2=O)CC1)C(=O)CO[C@@H]1CCN(C(=O)[C@@H](NC(=O)c2cc3cc(C(F)(F)P(=O)(O)O)ccc3s2)C(C)(C)C)[C@@H]1C(=O)N1CCO[C@H](c2ccccc2)C1 ; Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21]

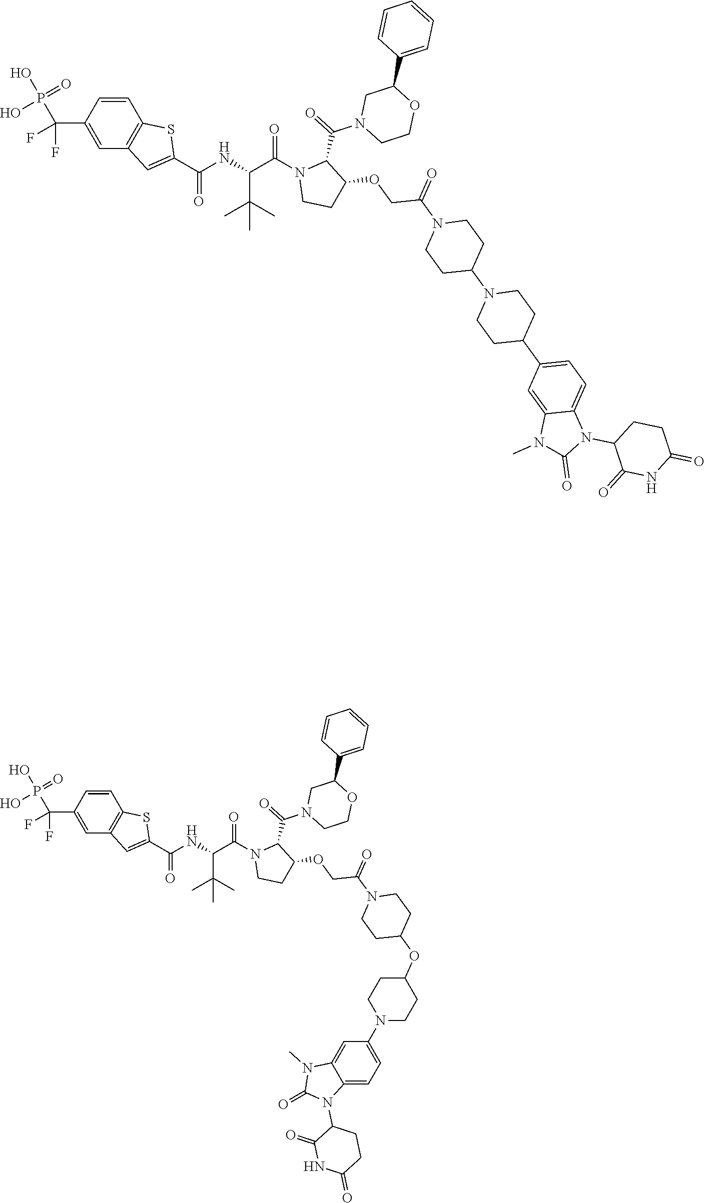 [SMILES: Cn1c(=O)n(C2CCC(=O)NC2=O)c2ccc(C3CCN(C4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21 ; Cn1c(=O)n(C2CCC(=O)NC2=O)c2ccc(N3CCC(OC4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21]

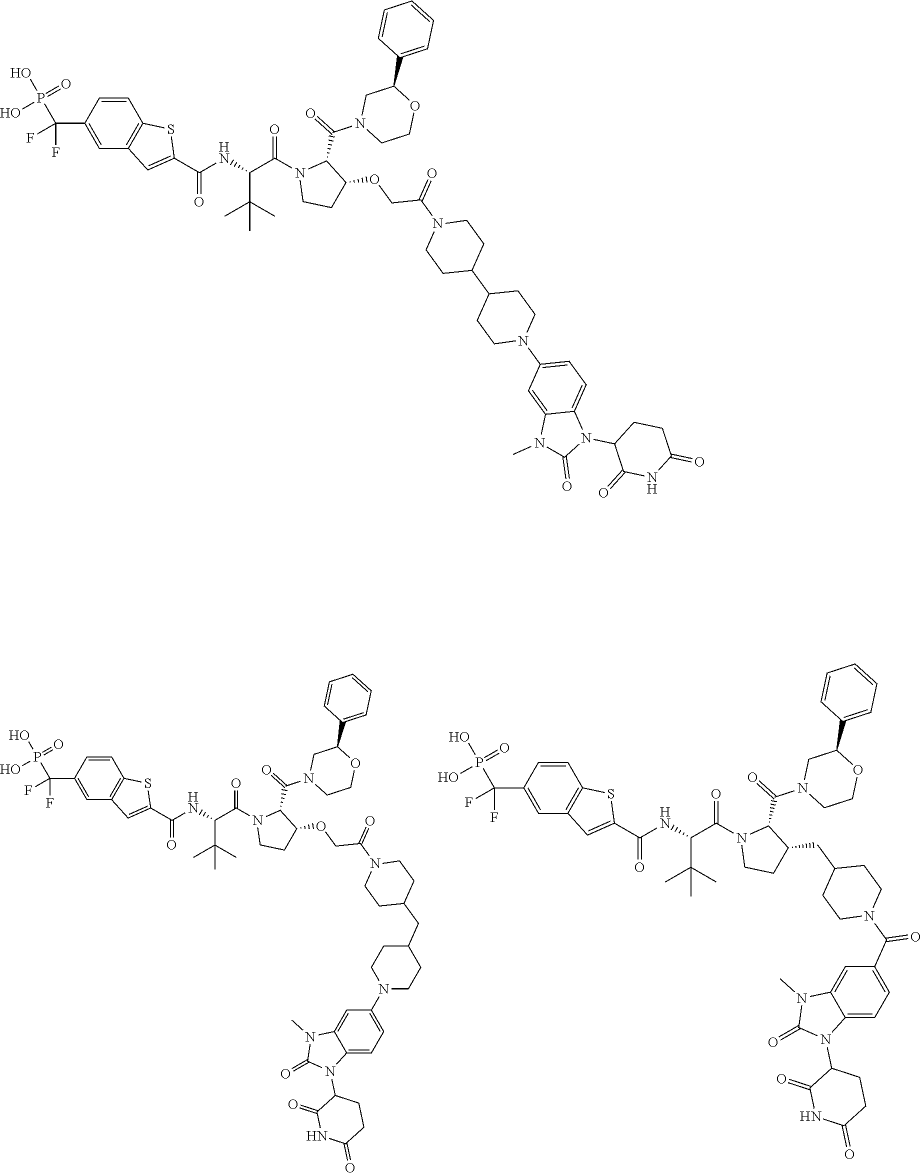 [SMILES: Cn1c(=O)n(C2CCC(=O)NC2=O)c2ccc(N3CCC(C4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21 ; Cn1c(=O)n(C2CCC(=O)NC2=O)c2ccc(N3CCC(CC4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21 ; Cn1c(=O)n(C2CCC(=O)NC2=O)c2ccc(C(=O)N3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21]

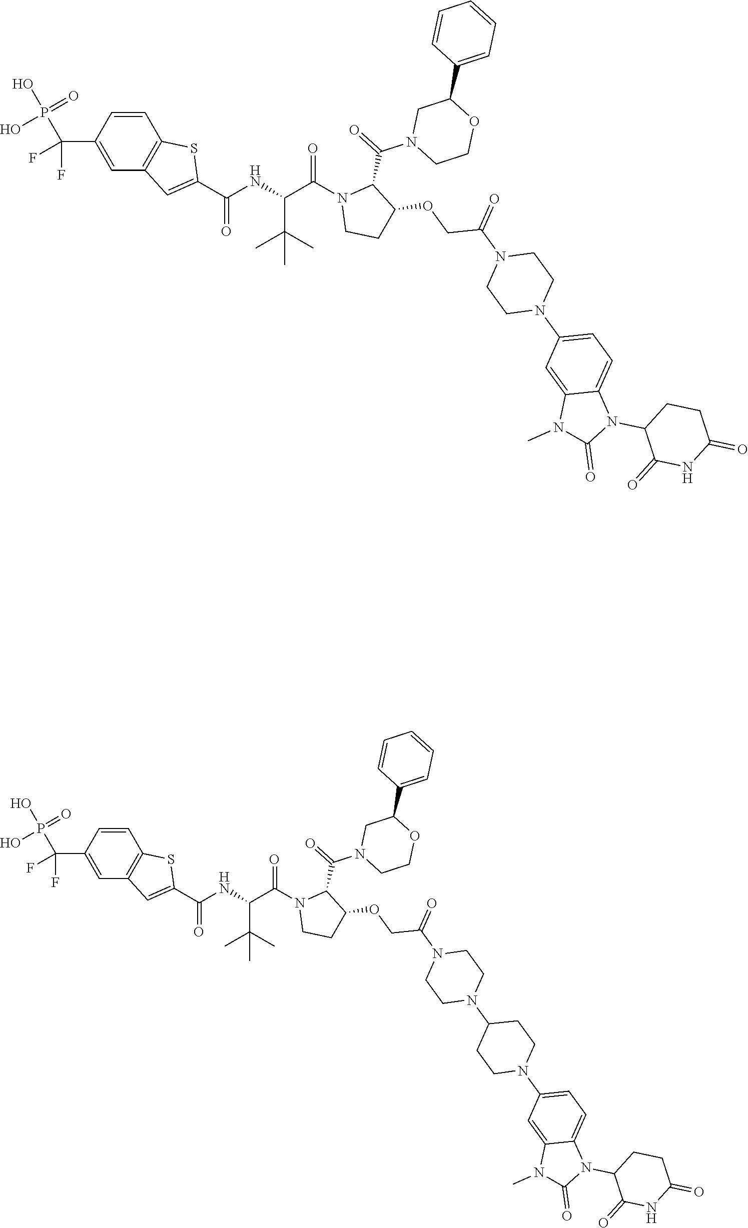 [SMILES: Cn1c(=O)n(C2CCC(=O)NC2=O)c2ccc(N3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(N3CCC(N4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21]

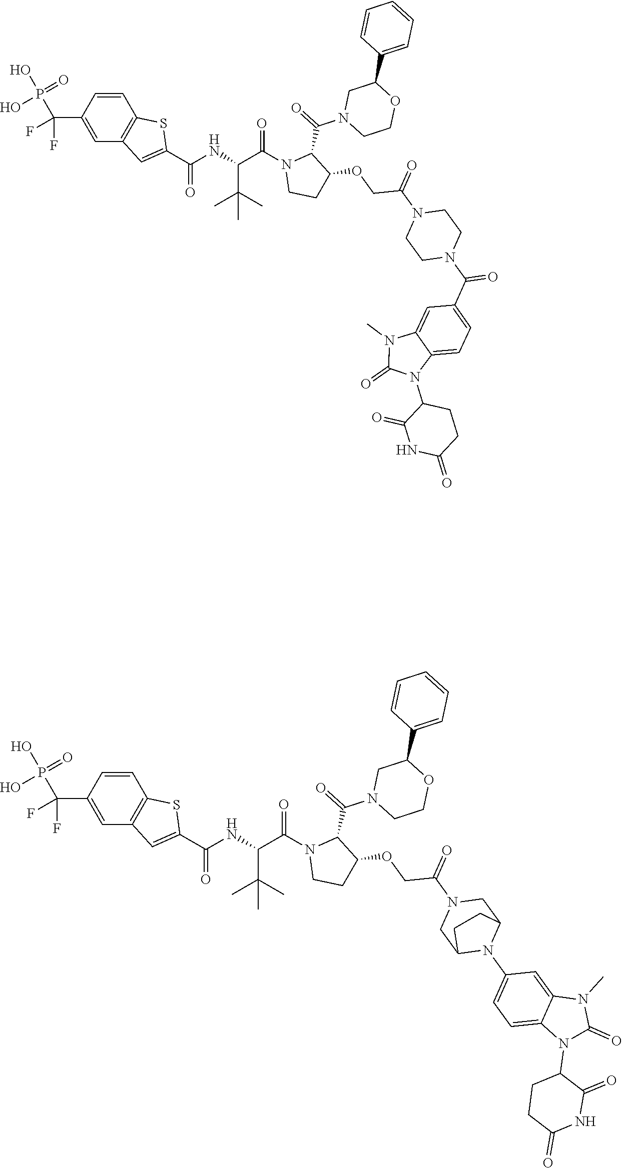 [SMILES: Cn1c(=O)n(C2CCC(=O)NC2=O)c2ccc(C(=O)N3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(N3C4CCC3CN(C(=O)CO[C@@H]3CCN(C(=O)[C@@H](NC(=O)c5cc6cc(C(F)(F)P(=O)(O)O)ccc6s5)C(C)(C)C)[C@@H]3C(=O)N3CCO[C@H](c5ccccc5)C3)C4)cc21]

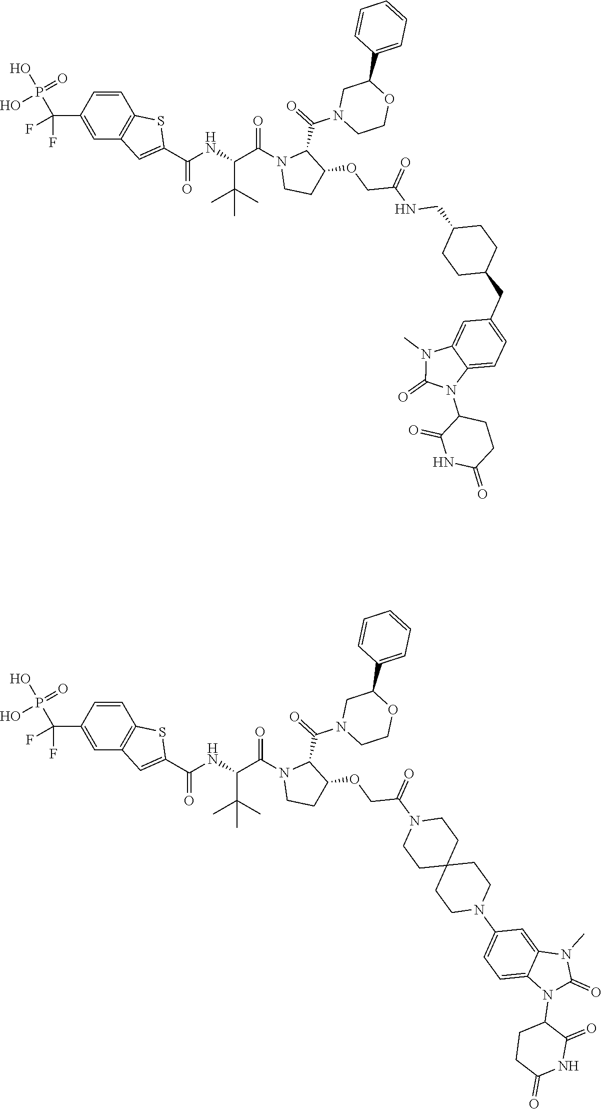 [SMILES: Cn1c(=O)n(C2CCC(=O)NC2=O)c2ccc(C[C@H]3CC[C@H](CNC(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(N3CCC4(CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21]

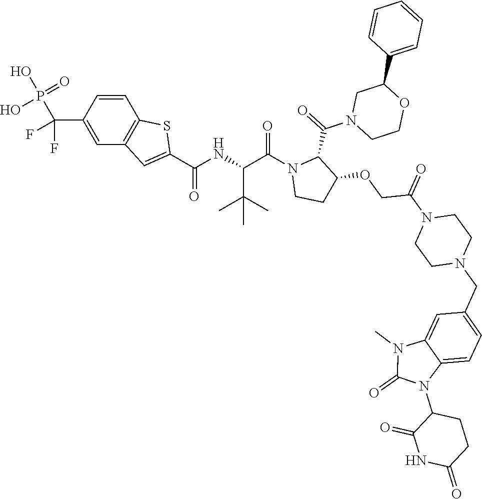 [SMILES: Cn1c(=O)n(C2CCC(=O)NC2=O)c2ccc(CN3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21]

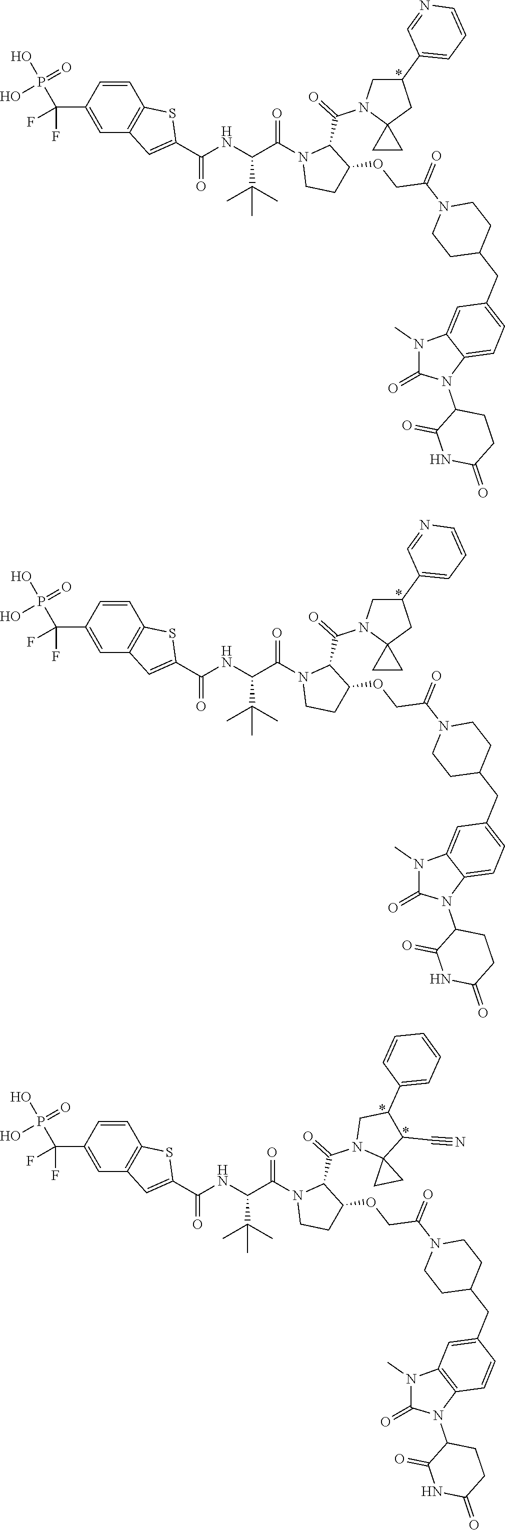 [SMILES: Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CC(c5cccnc5)CC45CC5)CC3)cc21 ; Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CC(c5cccnc5)CC45CC5)CC3)cc21 ; Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CC(c5ccccc5)C(C#N)C45CC5)CC3)cc21]

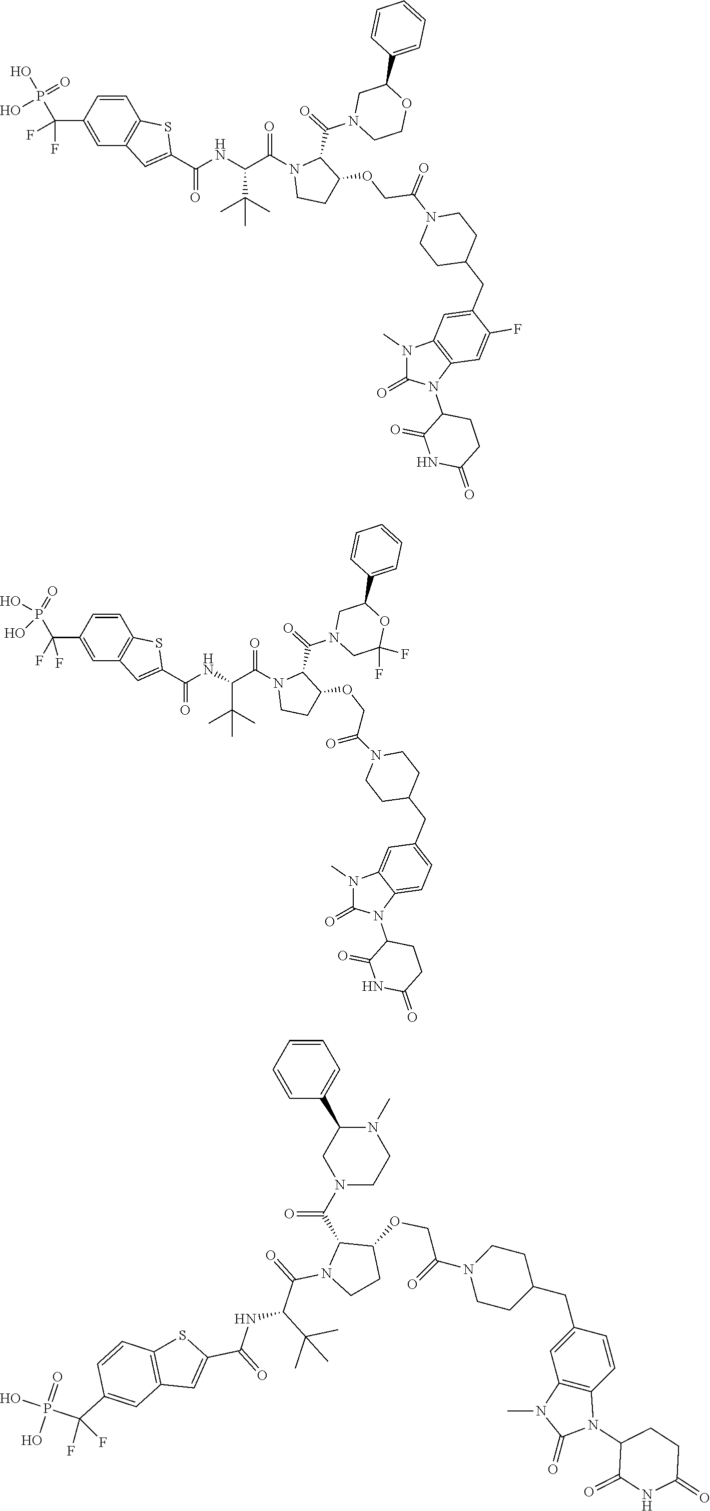 [SMILES: Cn1c(=O)n(C2CCC(=O)NC2=O)c2cc(F)c(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4C[C@@H](c5ccccc5)OC(F)(F)C4)CC3)cc21 ; CN1CCN(C(=O)[C@@H]2[C@H](OCC(=O)N3CCC(Cc4ccc5c(c4)n(C)c(=O)n5C4CCC(=O)NC4=O)CC3)CCN2C(=O)[C@@H](NC(=O)c2cc3cc(C(F)(F)P(=O)(O)O)ccc3s2)C(C)(C)C)C[C@H]1c1ccccc1]

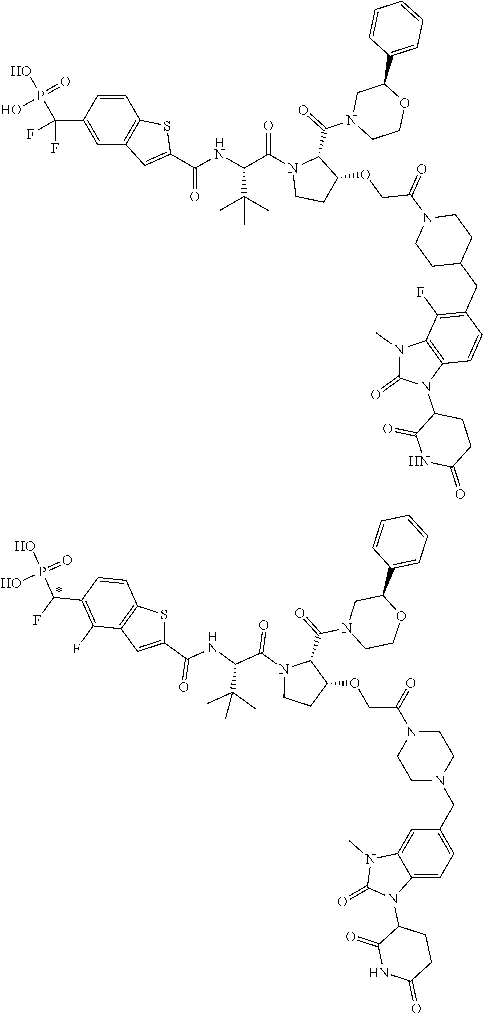 [SMILES: Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)c(F)c21 ; Cn1c(=O)n(C2CCC(=O)NC2=O)c2ccc(CN3CCN(C(=O)CO[C@@H]4CCN(C(=O)[C@@H](NC(=O)c5cc6c(F)c(C(F)P(=O)(O)O)ccc6s5)C(C)(C)C)[C@@H]4C(=O)N4CCO[C@H](c5ccccc5)C4)CC3)cc21]

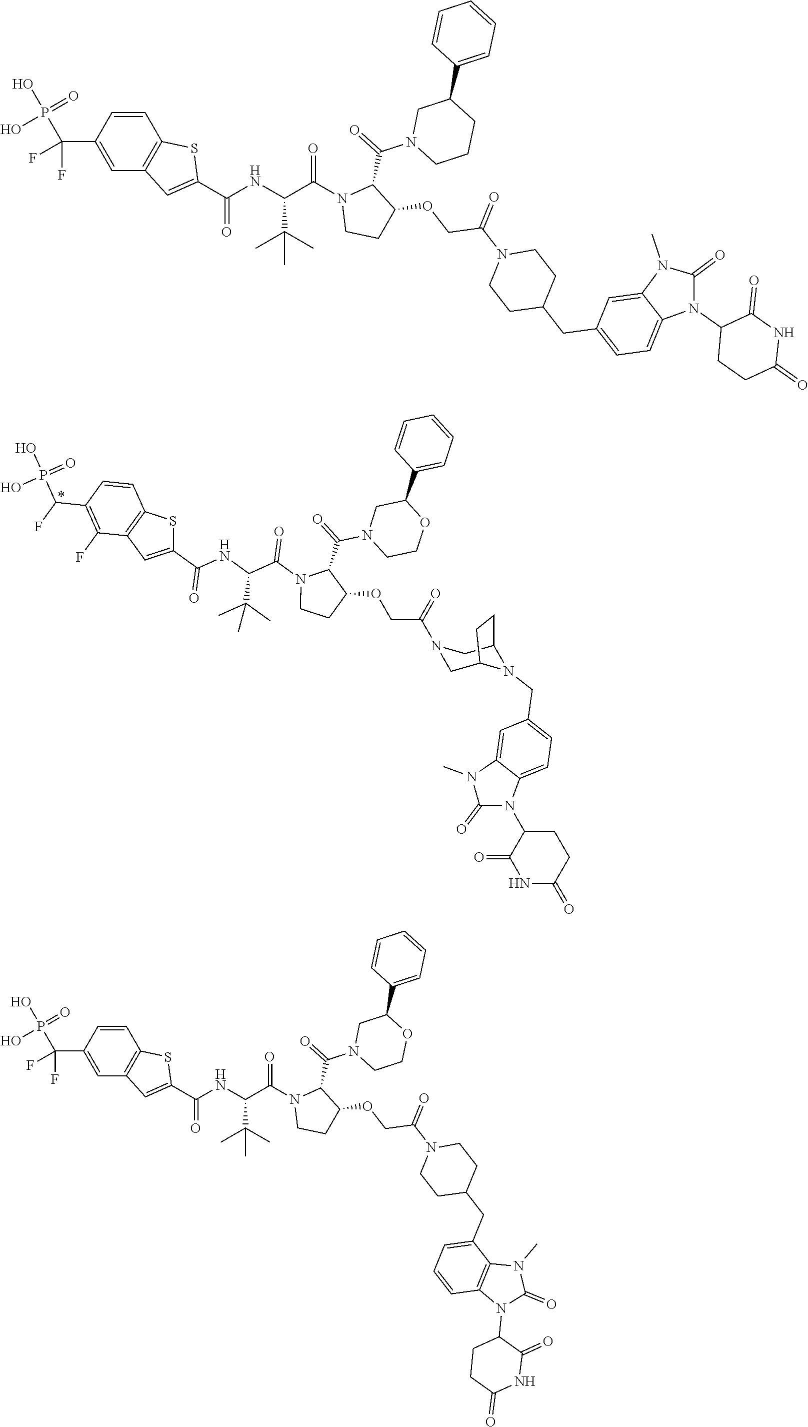 [SMILES: Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCC[C@H](c5ccccc5)C4)CC3)cc21 ; Cn1c(=O)n(C2CCC(=O)NC2=O)c2ccc(CN3C4CCC3CN(C(=O)CO[C@@H]3CCN(C(=O)[C@@H](NC(=O)c5cc6c(F)c(C(F)P(=O)(O)O)ccc6s5)C(C)(C)C)[C@@H]3C(=O)N3CCO[C@H](c5ccccc5)C3)C4)cc21 ; Cn1c(=O)n(C2CCC(=O)NC2=O)c2cccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)c21]

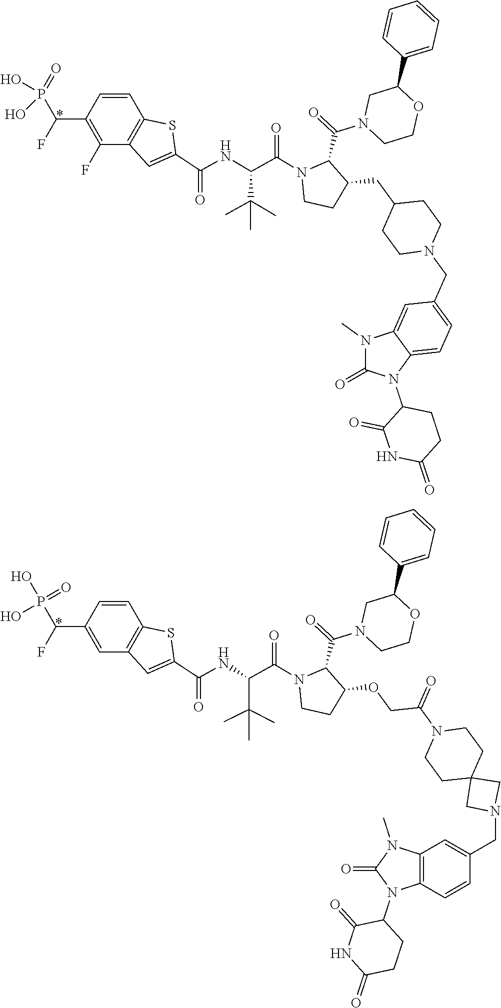

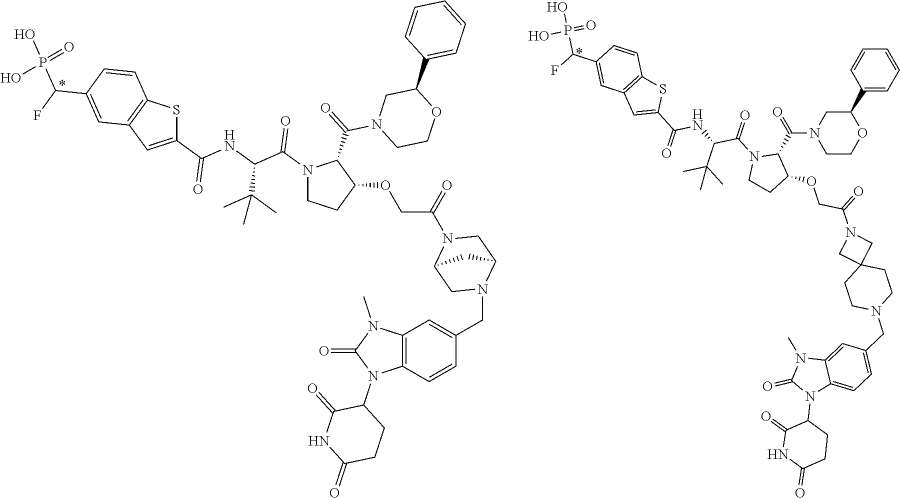 [SMILES: Cn1c(=O)n(C2CCC(=O)NC2=O)c2ccc(CN3C[C@@H]4C[C@H]3CN4C(=O)CO[C@@H]3CCN(C(=O)[C@@H](NC(=O)c4cc5cc(C(F)P(=O)(O)O)ccc5s4)C(C)(C)C)[C@@H]3C(=O)N3CCO[C@H](c4ccccc4)C3)cc21 ; Cn1c(=O)n(C2CCC(=O)NC2=O)c2ccc(CN3CCC4(CC3)CN(C(=O)CO[C@@H]3CCN(C(=O)[C@@H](NC(=O)c5cc6cc(C(F)P(=O)(O)O)ccc6s5)C(C)(C)C)[C@@H]3C(=O)N3CCO[C@H](c5ccccc5)C3)C4)cc21]

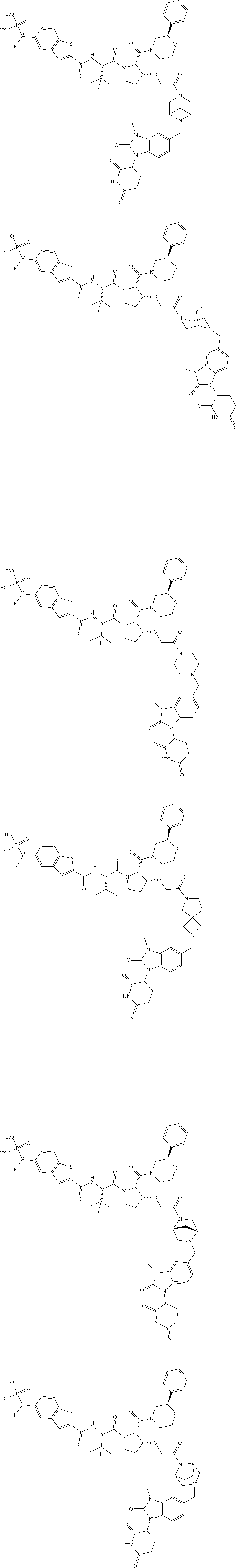

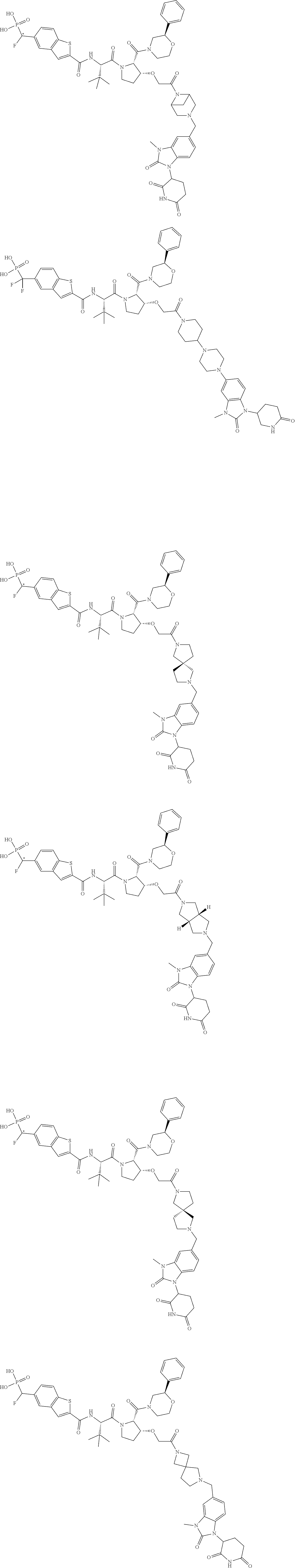

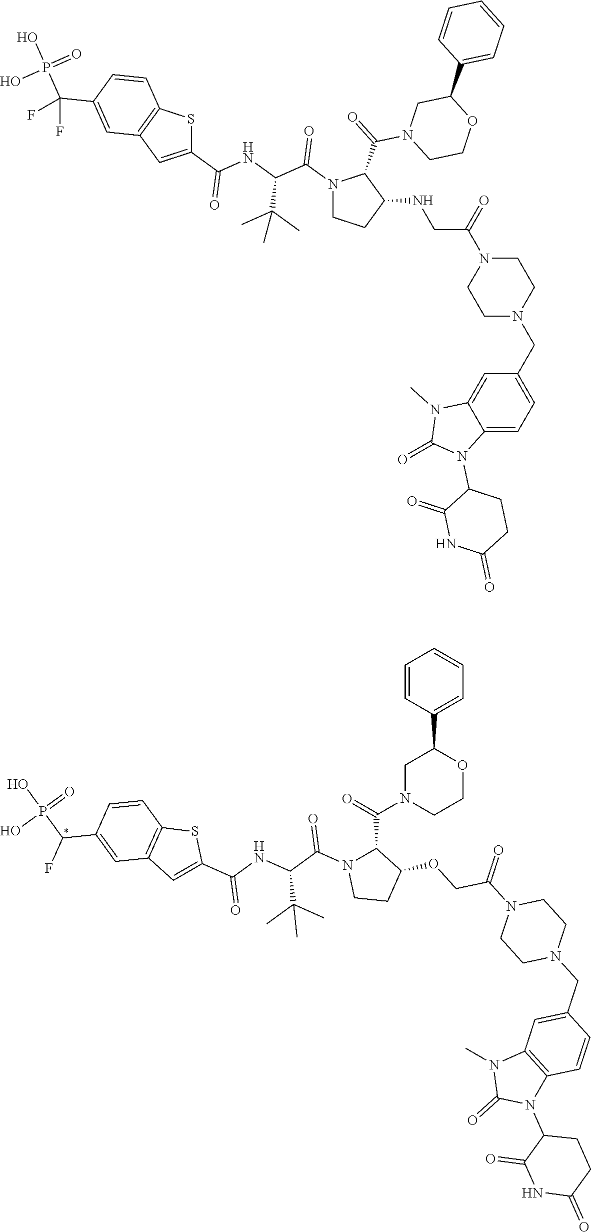 [SMILES: Cn1c(=O)n(C2CCC(=O)NC2=O)c2ccc(CN3CCN(C(=O)CN[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(CN3CCN(C(=O)CO[C@@H]4CCN(C(=O)[C@@H](NC(=O)c5cc6cc(C(F)P(=O)(O)O)ccc6s5)C(C)(C)C)[C@@H]4C(=O)N4CCO[C@H](c5ccccc5)C4)CC3)cc21]

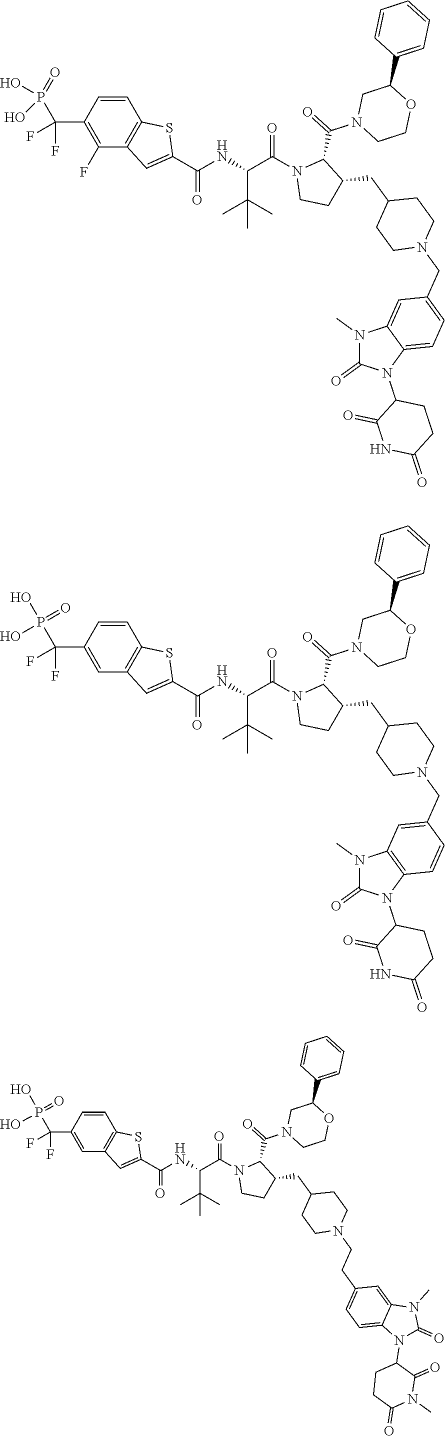 [SMILES: Cn1c(=O)n(C2CCC(=O)NC2=O)c2ccc(CN3CCC(C[C@@H]4CCN(C(=O)[C@@H](NC(=O)c5cc6c(F)c(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(CN3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; CN1C(=O)CCC(n2c(=O)n(C)c3cc(CCN4CCC(C[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)ccc32)C1=O]

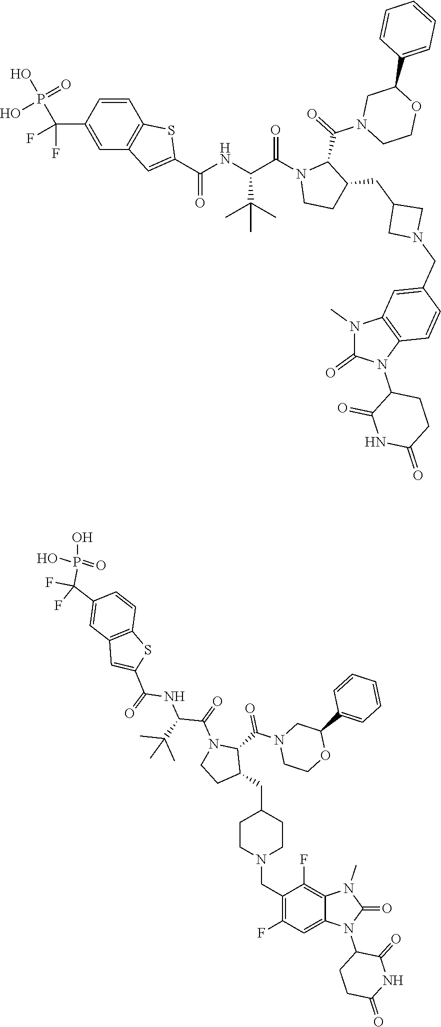 [SMILES: Cn1c(=O)n(C2CCC(=O)NC2=O)c2ccc(CN3CC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)C3)cc21 ; Cn1c(=O)n(C2CCC(=O)NC2=O)c2cc(F)c(CN3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)c(F)c21]

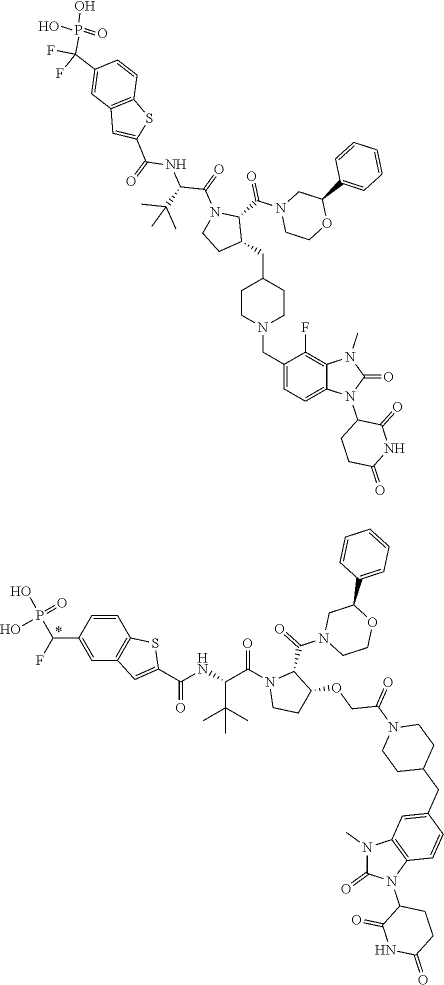 [SMILES: Cn1c(=O)n(C2CCC(=O)NC2=O)c2ccc(CN3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)c(F)c21 ; Cn1c(=O)n(C2CCC(=O)NC2=O)c2ccc(CC3CCN(C(=O)CO[C@@H]4CCN(C(=O)[C@@H](NC(=O)c5cc6cc(C(F)P(=O)(O)O)ccc6s5)C(C)(C)C)[C@@H]4C(=O)N4CCO[C@H](c5ccccc5)C4)CC3)cc21]

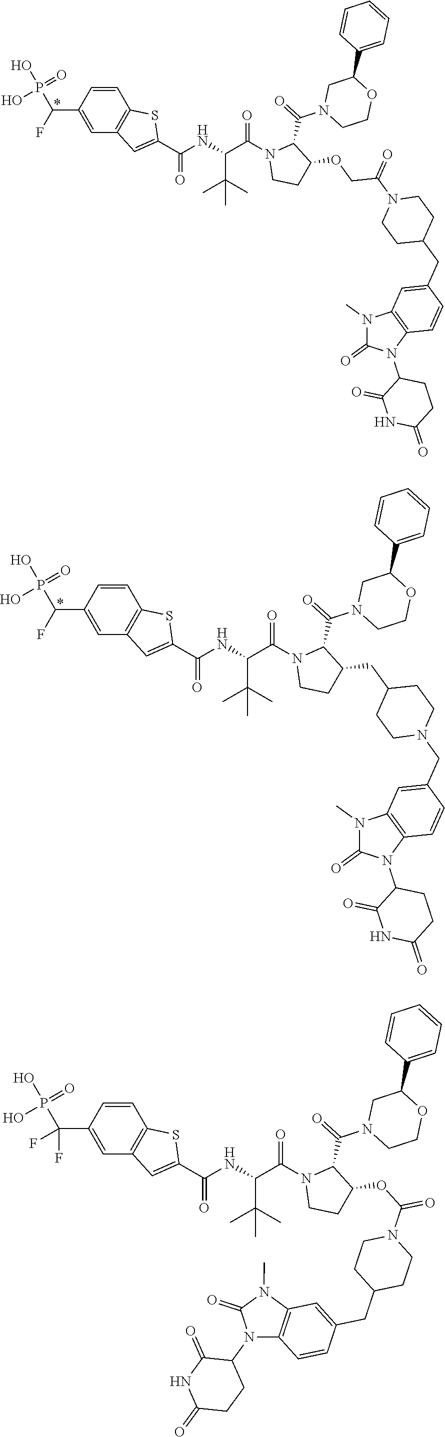

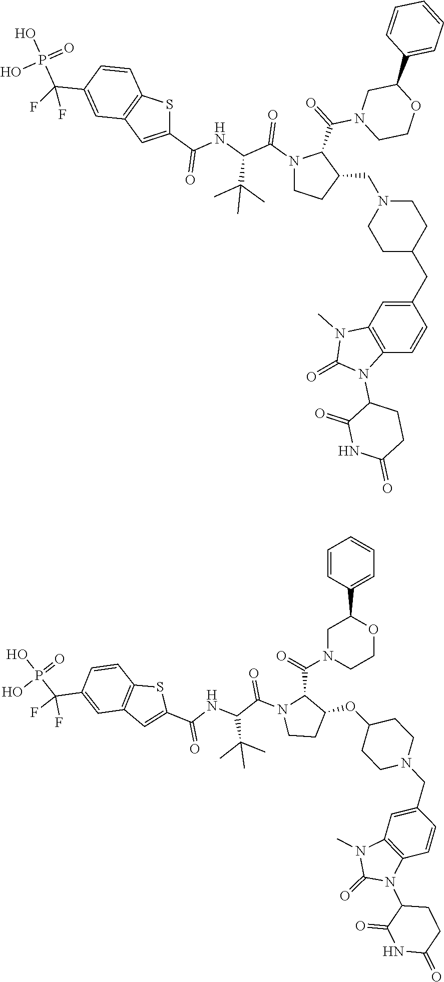

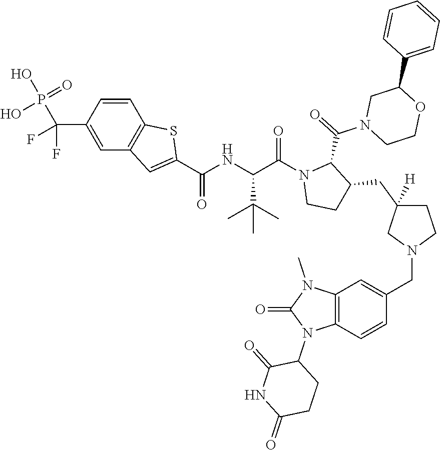 [SMILES: Cn1c(=O)n(C2CCC(=O)NC2=O)c2ccc(CN3CC[C@H](C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)C3)cc21]

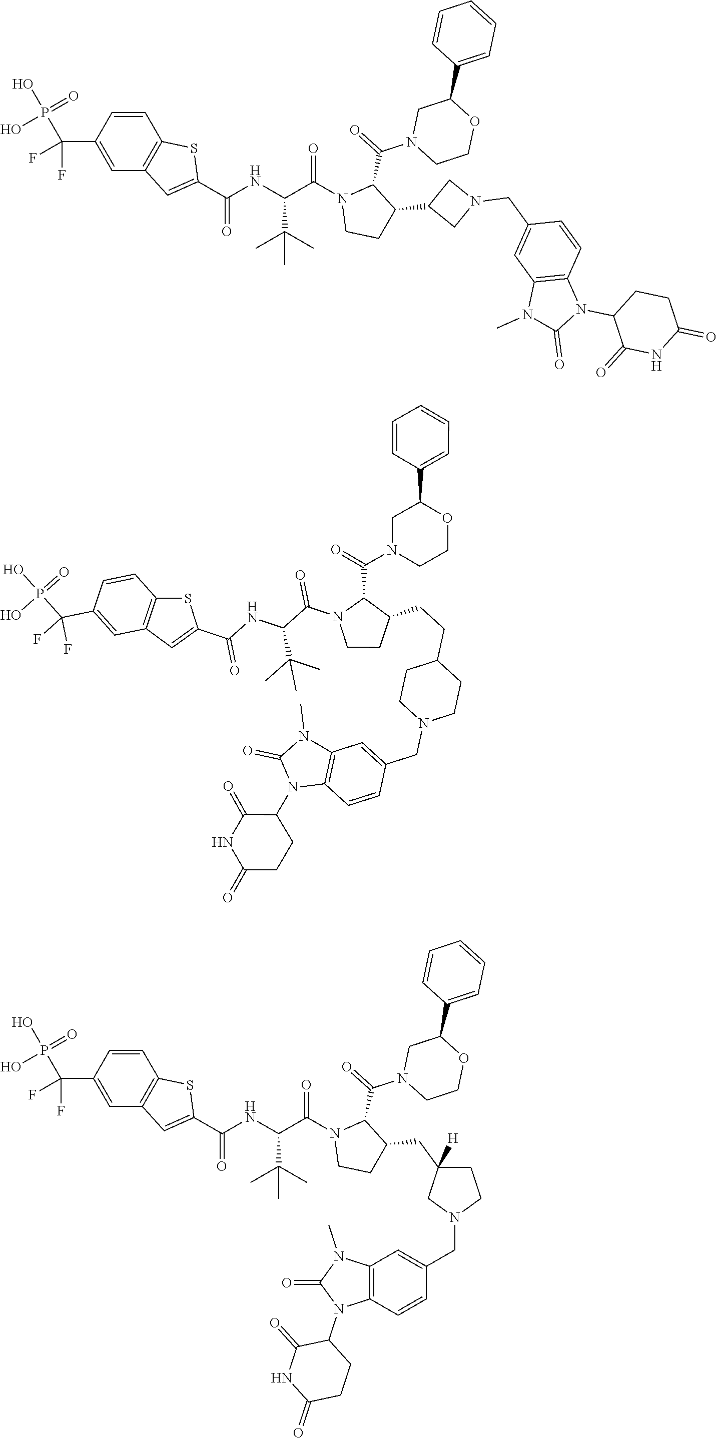 [SMILES: Cn1c(=O)n(C2CCC(=O)NC2=O)c2ccc(CN3CC([C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)C3)cc21 ; Cn1c(=O)n(C2CCC(=O)NC2=O)c2ccc(CN3CCC(CC[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(CN3CC[C@@H](C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)C3)cc21]

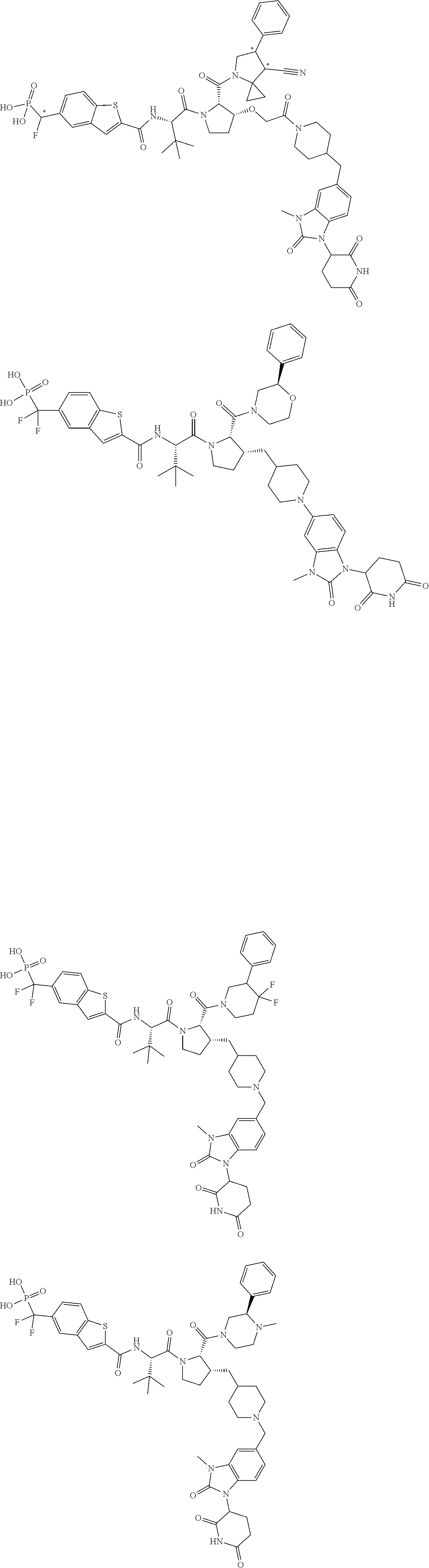

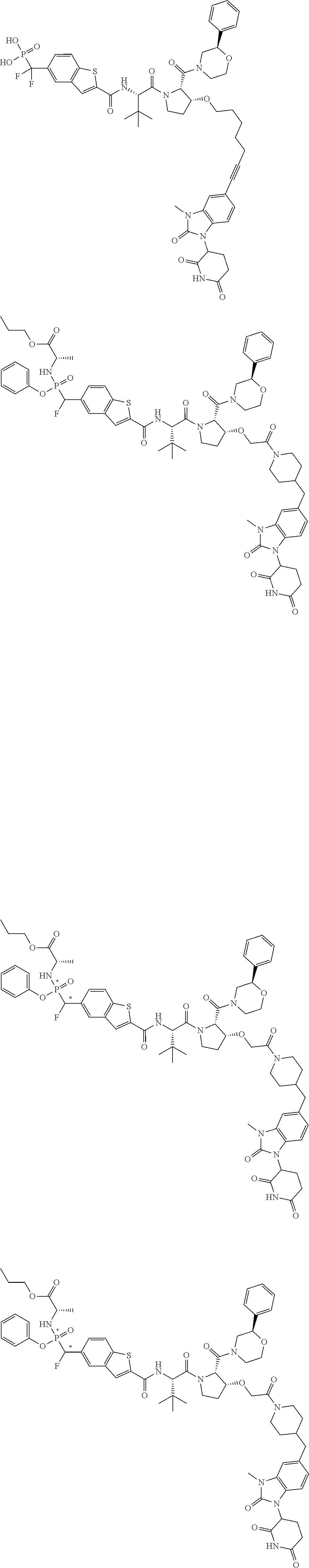

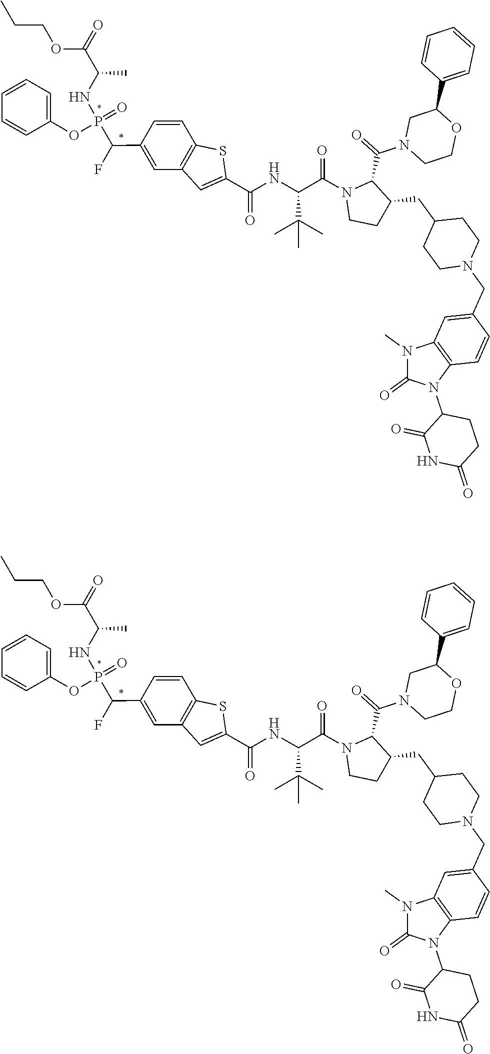 [SMILES: CCCOC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1 ; CCCOC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1]

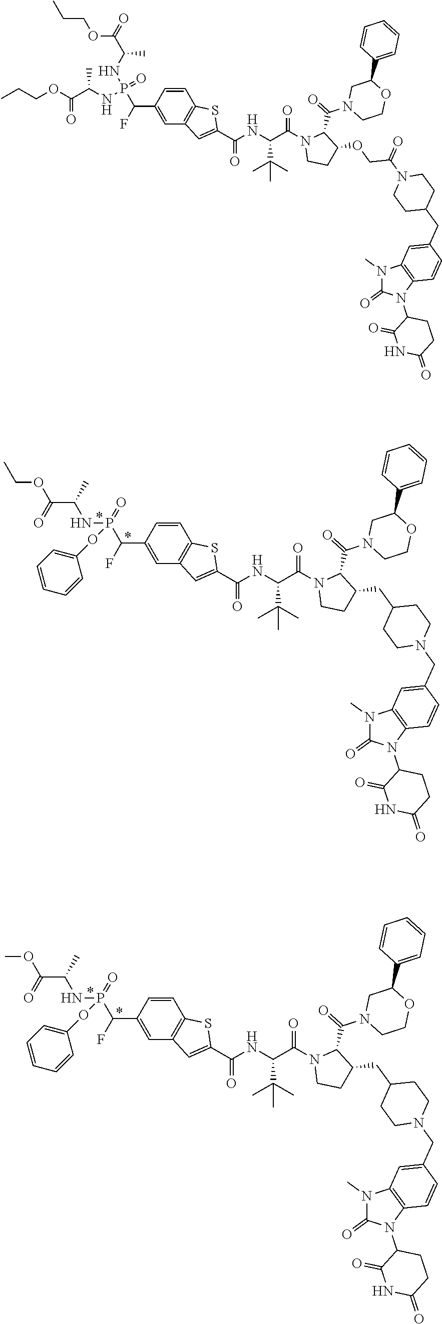

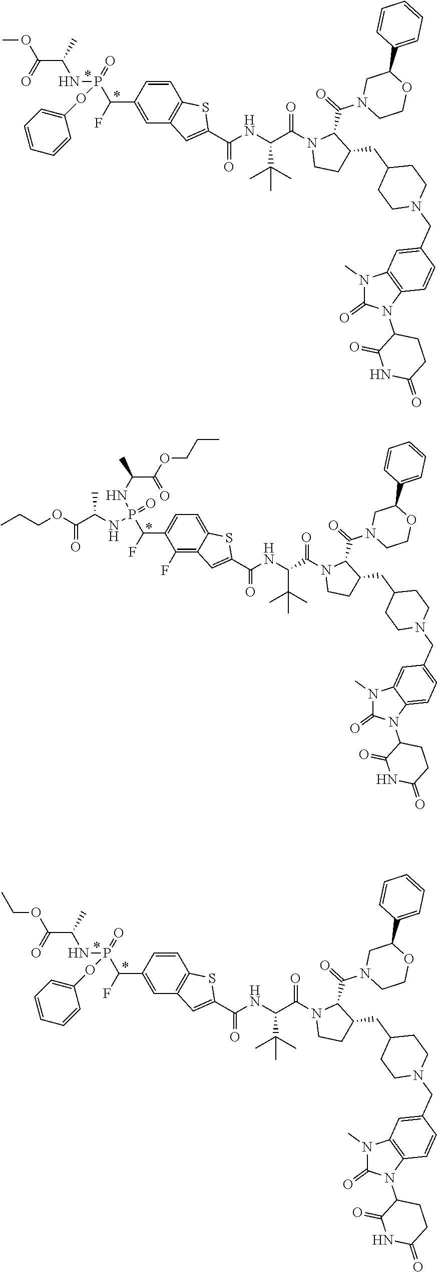

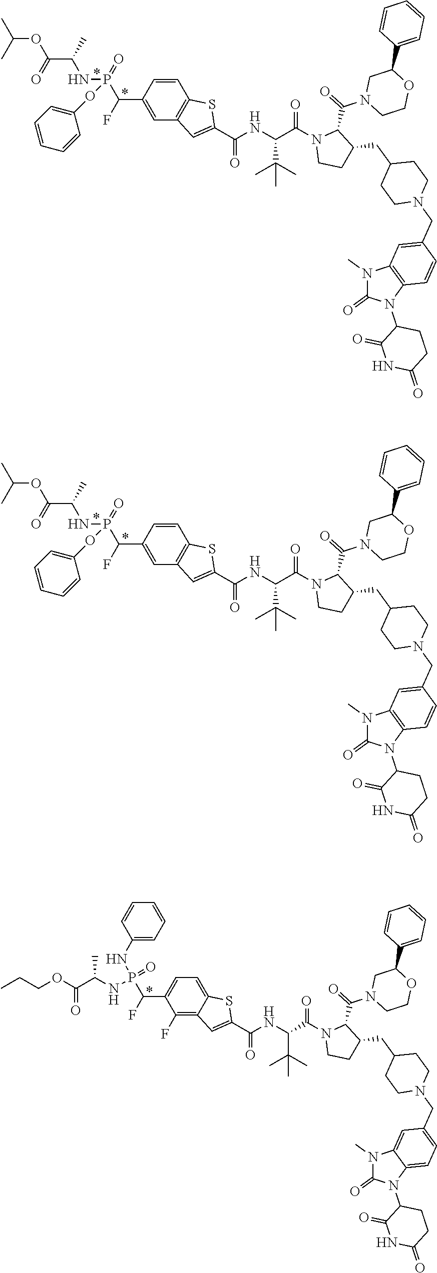

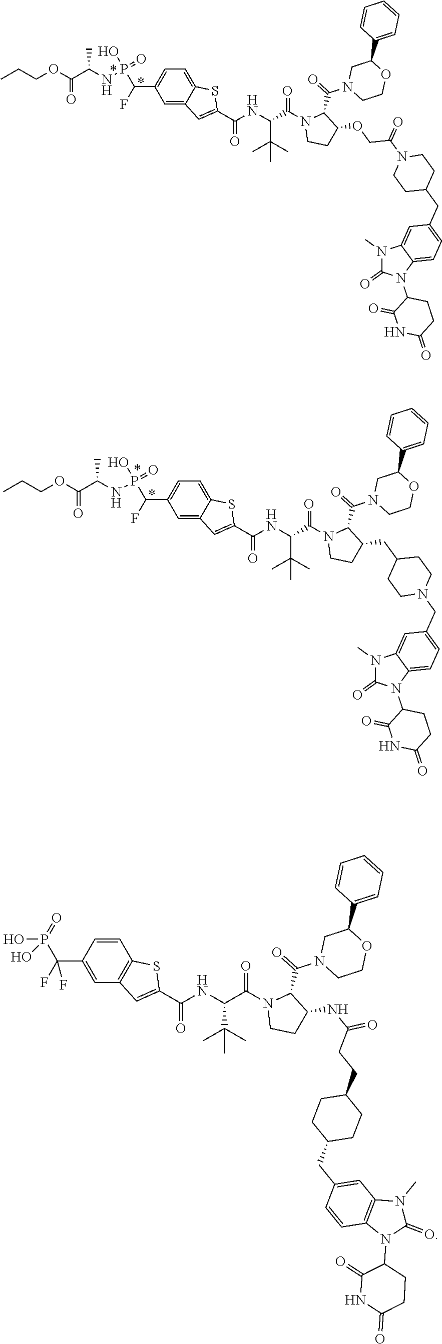

Also falling within the scope herein are the in vivo metabolic products of the compounds described herein. Such products may result for example from the oxidation, reduction, hydrolysis, amidation, esterification and the like of the administered compound, primarily due to enzymatic processes. Accordingly, included are novel and unobvious compounds produced by a process comprising contacting a compound with a mammal for a period of time sufficient to yield a metabolic product thereof. Such products typically are identified by preparing a radiolabelled (e.g., ¹⁴C or ³H) compound, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites is done in the same way as conventional drug metabolism studies. The conversion products, so long as they are not otherwise found in vivo, are useful in diagnostic assays for therapeutic dosing of the compounds even if they possess no activity of their own.

### IV. Pharmaceutical Compositions

Also disclosed herein are pharmaceutical compositions comprising a pharmaceutically effective amount of a compound of the present disclosure (e.g., a compound of Formula J, I, or Ia, or a pharmaceutically acceptable salt thereof, and a pharmaceutically acceptable carrier or excipient. Also provided herein is a pharmaceutical composition comprising a pharmaceutically effective amount of a compound of the present disclosure, or a pharmaceutically acceptable salt thereof, and a pharmaceutically acceptable carrier or excipient.

The compounds disclosed herein can be formulated with conventional carriers and excipients. Tablets can contain, for instance, excipients, glidants, fillers, binders, or a combination thereof. Aqueous formulations are prepared in sterile form, and when intended for delivery by other than oral administration generally will be isotonic. Exemplary excipients include, but are not limited to, those set forth in the “HANDBOOK OF PHARMACEUTICAL EXCIPIENTS” (1986). Excipients can include, for example, ascorbic acid and other antioxidants, chelating agents such as EDTA, carbohydrates such as dextran, hydroxyalkylcellulose, hydroxyalkylmethylcellulose, stearic acid, and combinations thereof. In some embodiments, the formulation is basic. In some embodiments, the formulation is acidic. In some embodiments, the formulation has a neutral pH. In some embodiments, the pH of the formulations is from 2 to 11 (e.g., 2, 3, 4, 5, 6, 7, 8, 9, 10, 11, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 4-11, 5-6, 5-7, 5-8, 5-9, 5-10, 5-11, 6-7, 6-8, 6-9, 6-10, 6-11, 7-8, 7-9, 7-10, 7-11, 8-9, 8-10, 8-11, 9-10, or 9-11).

In some embodiments, the compounds disclosed herein have pharmacokinetic properties (e.g., oral bioavailability) suitable for oral administration of the compounds. Formulations suitable for oral administration can, for instance, be presented as discrete units such as capsules, cachets or tablets, each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient can also be administered, for instance, as a bolus, electuary, or paste.

A tablet can be made by compression or molding, optionally with at least accessory ingredients. Compressed tablets can be prepared by compressing in a suitable machine the active ingredient in a free-flowing form such as, for instance, a powder or granules, optionally mixed with a binder, lubricant, inert diluent, preservative, surface active, dispersing agent, or a combination thereof. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom.

For infections of the eye or other external tissues (e.g., mouth and skin), the formulations can be applied as a topical ointment or cream containing the active ingredient(s) in an amount of, for example, 0.075 to 20% w/w (including active ingredient(s) in a range from 0.1% to 20% in increments of 0.1% w/w such as 0.6% w/w, 0.7% w/w, etc.), from 0.2% to 15% w/w, or from 0.5% to 10% w/w. When formulated in an ointment, the active ingredients can be employed in some embodiments with either a paraffinic or a water-miscible ointment base. Alternatively, the active ingredients can be formulated in a cream with an oil-in-water cream base.

In some embodiments, the aqueous phase of the cream base can include, for example, from 30% to 90% (e.g., 35%, 40%, 45%, 50%, 55%, 60%, 65%, 70%, 75%, 80%, 85%) w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. In some embodiments, the cream base can include, for instance, a compound that enhances absorption or penetration of the active ingredient through the skin or other affected areas. Examples of such dermal penetration enhancers include, but are not limited to, dimethyl sulfoxide and related analogs. In some embodiments, the cream or emulsion does not include water.

The oily phase of the emulsions can be constituted from known ingredients in a known manner. In some embodiments, the phase comprises merely an emulsifier (otherwise known as an emulgent). In some embodiments, the phase comprises a mixture of at least one emulsifier with a fat, an oil, or a combination thereof. In some embodiments, a hydrophilic emulsifier is included together with a lipophilic emulsifier that acts as a stabilizer. Together, the emulsifier(s) with or without stabilizer(s) can make up the so-called emulsifying wax, and the wax together with the oil and fat make up the so-called emulsifying ointment base that can form the oily dispersed phase of the cream formulations.

Emulgents and emulsion stabilizers suitable for use in the formulation can include, but are not limited to, TWEEN® 60, TWEEN® 80, SPAN® 80, cetostearyl alcohol, benzyl alcohol, myristyl alcohol, glyceryl mono-stearate, sodium lauryl sulfate, and combinations thereof.

The choice of suitable oils or fats for the formulation can be based on achieving the desired cosmetic properties. In some embodiments, the cream can be a non-greasy, non-staining, and washable product with suitable consistency to avoid leakage from tubes or other containers. In some embodiments, esters can be included, such as, for example, straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate, a blend of branched chain esters known as CRODAMOL® CAP, or a combination thereof. In some embodiments, high melting point lipids such as white soft paraffin and/or liquid paraffin or other mineral oils can be included.

In some embodiments, the compounds disclosed herein are administered alone. In some embodiments, the compounds disclosed herein are administered in pharmaceutical compositions. In some embodiments, the pharmaceutical compositions are for veterinary use. In some embodiments, the pharmaceutical compositions are for human use. In some embodiments, the pharmaceutical compositions disclosed herein include at least one additional therapeutic agent. In some embodiments, the pharmaceutical compositions disclosed herein include one or more additional therapeutic agents. In some embodiments, the one or more additional therapeutic agents is independently a chemotherapeutic agent, an immunotherapeutic agent, a hormonal agent, an anti-hormonal agent, a targeted therapy agent, or an anti-angiogenesis agent.

Pharmaceutical compositions disclosed herein can be in any form suitable for the intended method of administration. The pharmaceutical compositions disclosed herein can be presented in unit dosage form and can be prepared by any of the methods well known in the art of pharmacy. Exemplary techniques and formulations can be found, for instance, in REMINGTON'S PHARMACEUTICAL SCIENCES (Mack Publishing Co., Easton, PA). Such methods can include the step of bringing into association a compound disclosed herein with the carrier that constitutes at least accessory ingredients. In general, the formulations can be prepared by uniformly and intimately bringing into association the active ingredient with liquid carriers or finely divided solid carriers or both, and then, if necessary, shaping the product.

When used for oral use for example, tablets, troches, lozenges, aqueous or oil suspensions, dispersible powders or granules, emulsions, hard or soft capsules, solutions, syrups or elixirs can be prepared. Formulations intended for oral use can be prepared according to any method known to the art for the manufacture of pharmaceutical compositions and such formulations can contain at least agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients can be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets can be uncoated or can be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax can be employed.

Formulations for oral use can be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin, or olive oil.

Aqueous suspensions can contain the active materials in admixture with excipients suitable for the manufacture of aqueous suspensions. Such excipients can include, for instance, a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension can also contain, for example, at least preservatives such as ethyl or n-propyl p-hydroxy-benzoate, one or more coloring agents, one or more flavoring agents, one or more sweetening agents (such as sucrose or saccharin), or combinations thereof. Further non-limiting examples of suspending agents include cyclodextrin. In some embodiments, the suspending agent is sulfobutyl ether beta-cyclodextrin (SEB-beta-CD), for example CAPTISOL®.

Oil suspensions can be formulated by suspending the active ingredient in a vegetable oil (e.g., arachis oil, olive oil, sesame oil, coconut oil, or a combination thereof), a mineral oil such as liquid paraffin, or a combination thereof. The oral suspensions can contain, for instance, a thickening agent, such as beeswax, hard paraffin, cetyl alcohol, or a combination thereof. In some embodiments, sweetening agents, such as those set forth above, and/or flavoring agents, are added to provide a palatable oral preparation. In some embodiments, the formulations disclosed herein are preserved by the addition of an antioxidant such as ascorbic acid.

Dispersible powders and granules suitable for preparation of an aqueous suspension by the addition of water can provide the active ingredient in admixture with a dispersing or wetting agent, a suspending agent, a preservative, and combinations thereof. Suitable dispersing or wetting agents and suspending agents are exemplified by those disclosed above. Additional excipients, for example sweetening, flavoring and coloring agents, can also be present.

The pharmaceutical compositions can also be in the form of oil-in-water emulsions. The oily phase can be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion can also contain sweetening and flavoring agents. Syrups and elixirs can be formulated with sweetening agents, such as for instance, glycerol, sorbitol or sucrose. Such formulations can also contain, for instance, a demulcent, a preservative, a flavoring, a coloring agent, or a combination thereof.

The pharmaceutical compositions can also include a permeation enhancer that promote the transport of the compounds across the intestinal mucosa by increasing paracellular or transcellular permeation. Various permeation enhancers and methods for the oral delivery of therapeutic agents is described in Brayden, D. J., Mrsny, R. J., 2011. Oral peptide delivery: prioritizing the leading technologies. Ther. Delivery 2 (12), 1567-1573. Examples of absorption enhancers may include Bile salts, fatty acids, surfactants (anionic, cationic, and nonanionic) chelators, Zonular OT, esters, cyclodextrin, dextran sulfate, azone, crown ethers, EDTA, sucrose esters, and phosphotidyl choline, for example. Although absorption enhancers are not typically carriers by themselves, they are also widely associated with other carriers to improve oral bioavailability by transporting of compounds and therapeutics across the intestinal mucosa. Such substances can be added to the pharmaceutical composition as excipients or incorporated to form non specific interactions with the intended compound.

Other permeation enhancers can include sodium salts of medium chain fatty acids (MCFAS). Representative MCFAS include, but are not limited to, sodium caprate, a salt of capric acid, which comprises 2-3% of the fatty acids in the milk fat fraction. The permeation properties of another dietary MCFAS, sodium caprylate (8-carbon), were shown in vitro to be lower when compared to sodium caprate. In some embodiments, the permeation enhancer can be sodium caprylate. In some embodiments, the permeation enhancer can be sodium caprate. In some embodiments, the permeation enhancer can be sodium 8-(2-hydroxybenzamido)octanoate (SNAC).

The pharmaceutical compositions can be in the form of a sterile injectable or intravenous preparation, such as a sterile injectable aqueous or oleaginous suspension. This suspension can be formulated according to the known art using those suitable dispersing or wetting agents and suspending agents which have been mentioned above. The sterile injectable or intravenous preparation can also be a sterile injectable solution or suspension in a non-toxic parenterally acceptable diluent or solvent, such as a solution in 1,3-butane-diol or prepared as a lyophilized powder. Among the acceptable vehicles and solvents that can be employed are water, Ringer's solution and isotonic sodium chloride solution. In addition, sterile fixed oils can be employed as a solvent or suspending medium. For this purpose, any bland fixed oil can be employed including synthetic mono- or diglycerides. In addition, fatty acids such as oleic acid can likewise be used in the preparation of injectables. Among the acceptable vehicles and solvents that can be employed include, but are not limited to, water, Ringer's solution isotonic sodium chloride solution, and hypertonic sodium chloride solution.

The amount of active ingredient that can be combined with the carrier material to produce a single dosage form will vary depending upon the host treated and the particular mode of administration. For example, a time-release formulation intended for oral administration to humans can contain approximately 1 mg to 2000 mg of active material compounded with an appropriate and convenient amount of carrier material, which can vary from 5% to 95% of the total formulations (weight:weight). For example, a time-release formulation intended for oral administration to humans can contain approximately 1 mg to 1000 mg of active material compounded with an appropriate and convenient amount of carrier material, which can vary from 5% to 95% of the total formulations (weight:weight). The pharmaceutical composition can be prepared to provide easily measurable amounts for administration. For example, an aqueous solution intended for intravenous infusion can contain from 3 μg to 500 μg of the active ingredient per milliliter of solution in order that infusion of a suitable volume at a rate of 30 mL/hr can occur.

Formulations suitable for topical administration to the eye also include eye drops wherein the active ingredient is dissolved or suspended in a suitable carrier, especially an aqueous solvent for the active ingredient. In some embodiments, the compounds disclosed herein are included in the pharmaceutical compositions disclosed herein in a concentration of 0.5% to 20% (e.g., 0.5% to 10%, 1.5% w/w).

Formulations suitable for topical administration in the mouth include lozenges can comprise an active ingredient (i.e., a compound disclosed herein and/or additional therapeutic agents) in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration can be presented as a suppository with a suitable base comprising for example cocoa butter or a salicylate.

Formulations suitable for vaginal administration can be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions that can contain anti-oxidants, buffers, bacteriostats and solutes that render the formulation isotonic with the blood of the intended recipient; and aqueous and non-aqueous sterile suspensions that can include suspending agents and thickening agents.

The formulations can be presented in unit-dose or multi-dose containers, for example, sealed ampoules and vials, and can be stored in a freeze-dried (lyophilized) condition requiring only the addition of the sterile liquid carrier, for example water for injection, immediately before use. Extemporaneous injection solutions and suspensions are prepared from sterile powders, granules and tablets of the kind previously described. Preferred unit-dosage formulations are those containing a daily dose or unit daily sub-dose, as herein above recited, or an appropriate fraction thereof, of the active ingredient.

It should be understood that in addition to the ingredients particularly mentioned above the formulations can include other agents conventional in the art having regard to the type of formulation in question, for example those suitable for oral administration can include flavoring agents.

Further provided are veterinary formulations comprising a compound disclosed herein together with a veterinary carrier therefor.

Veterinary carriers are materials useful for the purpose of administering the formulation and can be solid, liquid or gaseous materials which are otherwise inert or acceptable in the veterinary art and are compatible with the active ingredient. These veterinary formulations can be administered orally, parenterally, or by any other desired route.

Compounds herein are used to provide controlled release pharmaceutical compositions containing as active ingredient one or more of the compounds (“controlled release formulations”) in which the release of the active ingredient can be controlled and regulated to allow less frequency dosing or to improve the pharmacokinetic or toxicity profile of a given active ingredient.

Effective dose of active ingredient depends at least on the nature of the condition being treated, toxicity, whether the compound is being used prophylactically (lower doses) or against an active disease, the method of delivery, and the pharmaceutical composition, and will be determined by the clinician using conventional dose escalation studies. In some embodiments, the effective dose is from 0.0001 to 100 mg/kg body weight per day; for instance, from 10 to 30 mg/kg body weight per day; from 15 to 25 mg/kg body weight per day; from 10 to 15 mg/kg body weight per day; or from 20 to 30 mg/kg body weight per day. For example, the daily candidate dose for an adult human of approximately 70 kg body weight can range from 1 mg to 2000 mg (e.g., from 5 mg to 500 mg, from 500 mg to 1000 mg, from 1000 mg to 1500 mg, from 1500 mg to 2000 mg), and can take the form of single or multiple doses. For example, the daily candidate dose for an adult human of approximately 70 kg body weight can range from 1 mg to 1000 mg (e.g., from 5 mg to 500 mg), and can take the form of single or multiple doses.

### V. Kits

Also provided herein are kits that include a compound disclosed herein or a pharmaceutically acceptable salt thereof. In some embodiments the kits described herein can comprise a label and/or instructions for use of the compound in the treatment of a disease or condition in a subject (e.g., human) in need thereof. In some embodiments, the disease or condition is an inflammatory condition such as immune mediated diseases or conditions, IL-4/IL-13 mediated conditions, dermatology and allergic disease indications, and atopic dermatitis.

In some embodiments, the kits can also comprise one or more additional therapeutic agents and/or instructions for use of additional therapeutic agents in combination with the compound disclosed herein in the treatment of the disease or condition in a subject (e.g., human) in need thereof.

In some embodiments, the kits provided herein comprise individual dose units of a compound as described herein, or a pharmaceutically acceptable salt, racemate, enantiomer, diastereomer, tautomer, polymorph, pseudopolymorph, amorphous form, hydrate or solvate thereof. Examples of individual dosage units can include pills, tablets, capsules, prefilled syringes or syringe cartridges, IV bags, inhalers, nebulizers etc., each comprising a therapeutically effective amount of the compound in question, or a pharmaceutically acceptable salt, racemate, enantiomer, diastereomer, tautomer, polymorph, pseudopolymorph, amorphous form, hydrate or solvate thereof. In some embodiments, the kit can contain a single dosage unit and in others multiple dosage units are present, such as the number of dosage units required for a specified regimen or period.

Also provided are articles of manufacture that include a compound disclosed herein, or a pharmaceutically acceptable salt, stereoisomer, mixture of stereoisomers or tautomer thereof; and a container. In some embodiments, the container of the article of manufacture is a vial, jar, ampoule, preloaded syringe, blister package, tin, can, bottle, box, an intravenous bag, an inhaler, or a nebulizer.

### VI. Administration

One or more of the compounds of Formula J, I, or Ia, (herein referred to as the active ingredients) are administered by any route appropriate to the condition to be treated. Suitable routes include oral, rectal, nasal, pulmonary (e.g., inhaled), topical (including buccal and sublingual), vaginal and parenteral (including subcutaneous, intramuscular, intravenous, intradermal, intrathecal and epidural), and the like. It will be appreciated that the route may vary with for example the condition of the recipient. In some embodiments, one or more compounds herein are orally bioavailable and can be dosed orally.

The compounds of the present disclosure (also referred to herein as the active ingredients), can be administered by any route appropriate to the condition to be treated.

Suitable routes include oral, rectal, nasal, topical (including buccal and sublingual), transdermal, pulmonary, vaginal and parenteral (including subcutaneous, intramuscular, intravenous, intradermal, intrathecal and epidural), and the like. It will be appreciated that the route may vary with for example the condition of the recipient.

A compound of the present disclosure may be administered to an individual in accordance with an effective dosing regimen for a desired period of time or duration, such as at least about one month, at least about 2 months, at least about 3 months, at least about 6 months, or at least about 12 months or longer. In some embodiments, the compound is administered on a daily or intermittent schedule for the duration of the individual's life.

The dosage or dosing frequency of a compound of the present disclosure may be adjusted over the course of the treatment, based on the judgment of the administering physician.

The compound may be administered to an individual (e.g., a human) in an effective amount. In some embodiments, the compound is administered once daily.

The compound can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 mg to about 30 mg per day, or such as from about 30 mg to about 300 mg per day.

A compound of the present disclosure may be combined with one or more additional therapeutic agents in any dosage amount of the compound of the present disclosure (e.g., from about 1 mg to about 1000 mg of compound). Therapeutically effective amounts may include from about 1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose. Other therapeutically effective amounts of the compound of the present disclosure are about 100, about 125, about 150, about 175, about 200, about 225, about 250, about 275, about 300, about 325, about 350, about 375, about 400, about 425, about 450, about 475, or about 500 mg per dose. Other therapeutically effective amounts of the compound of the present disclosure are about 100 mg per dose, or about 125, about 150, about 175, about 200, about 225, about 250, about 275, about 300, about 325, about 350, about 375, about 400, about 425, about 450, or about 500 mg per dose. A single dose can be administered hourly, daily, or weekly. For example, a single dose can be administered once about every 1 hour, about 2, about 3, about 4, about 6, about 8, about 12, about 16 or once about every 24 hours. A single dose can also be administered once about every 1 day, about 2, about 3, about 4, about 5, about 6, or once about every 7 days. A single dose can also be administered once about every 1 week, about 2, about 3, or once about every 4 weeks. In some embodiments, a single dose can be administered once about every week. A single dose can also be administered once about every month.

Other therapeutically effective amounts of the compound of the present disclosure are about 20, about 25, about 30, about 35, about 40, about 45, about 50, about 55, about 60, about 65, about 70, about 75, about 80, about 85, about 90, about 95, or about 100 mg per dose.

The frequency of dosage of the compound of the present disclosure can be determined by the needs of the individual patient and can be, for example, once per day or twice, or more times, per day. Administration of the compound continues for as long as necessary to treat the disease or condition. For example, a compound can be administered to a human having an inflammatory condition for a period of from about 20 days to about 180 days or, for example, for a period of from about 20 days to about 90 days or, for example, for a period of from about 30 days to about 60 days.

Administration can be intermittent, with a period of several or more days during which a patient receives a daily dose of the compound of the present disclosure followed by a period of several or more days during which a patient does not receive a daily dose of the compound. For example, a patient can receive a dose of the compound every other day, or three times per week. Again by way of example, a patient can receive a dose of the compound each day for a period of from about 1 to about 14 days, followed by a period of about 7 to about 21 days during which the patient does not receive a dose of the compound, followed by a subsequent period (e.g., from about 1 to about 14 days) during which the patient again receives a daily dose of the compound. Alternating periods of administration of the compound, followed by non-administration of the compound, can be repeated as clinically required to treat the patient.

In some embodiments, pharmaceutical compositions comprising a compound of the present disclosure, or a pharmaceutically acceptable salt thereof, in combination with one or more (e.g., one, two, three, four, one or two, one to three, or one to four) additional therapeutic agents, and a pharmaceutically acceptable excipient are provided.

In some embodiments, kits comprising a compound of the present disclosure, or a pharmaceutically acceptable salt thereof, in combination with one or more (e.g., one, two, three, four, one or two, one to three, or one to four) additional therapeutic agents are provided.

In some embodiments, a compound of the present disclosure, or a pharmaceutically acceptable salt thereof, is combined with one, two, three, four or more additional therapeutic agents. In some embodiments, a compound of the present disclosure, or a pharmaceutically acceptable salt thereof, is combined with two additional therapeutic agents. In some embodiments, a compound of the present disclosure, or a pharmaceutically acceptable salt thereof, is combined with three additional therapeutic agents. In some embodiments, a compound of the present disclosure, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In some embodiments, when a compound of the present disclosure is combined with one or more additional therapeutic agents as described herein, the components of the composition are administered as a simultaneous or sequential regimen. When administered sequentially, the combination may be administered in two or more administrations.

In some embodiments, a compound of the present disclosure is combined with one or more additional therapeutic agents in a unitary dosage form for simultaneous administration to a patient, for example as a solid dosage form for oral administration.

In some embodiments, a compound of the present disclosure is co-administered with one or more additional therapeutic agents.

In order to prolong the effect of a compound of the present disclosure, it is often desirable to slow the absorption of a compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending a compound in an oil vehicle. Injectable depot forms are made by forming microencapsule matrices of a compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping a compound in liposomes or microemulsions that are compatible with body tissues.

### VII. Methods of Use

The disclosure further relates to the use of compounds disclosed herein for the treatment and/or prophylaxis of diseases and/or conditions through modulation of signal transducer and activator of transcription 6 (STAT6) protein activity. Further, the present disclosure relates to the use of said compounds of Formula J, I, or Ia for the preparation of a medicament for the treatment and/or prophylaxis of inflammatory conditions.

Medicaments as referred to herein can be prepared by conventional processes, including the combination of a compound according to the present disclosure and a pharmaceutically acceptable carrier.

In some embodiments, the present disclosure provides a method of treating an immune mediated disease or condition in a subject in need thereof, the method comprising administering to the subject a therapeutically effective amount of a compound of the present disclosure, or a pharmaceutically acceptable salt thereof, or a pharmaceutical composition of the present disclosure.

In some embodiments, the present disclosure provides a method of modulating a signal transducer and activator of transcription 6 (STAT6) protein activity in a subject in need thereof, the method comprising administering to the subject a therapeutically effective amount of a compound of the present disclosure, or a pharmaceutically acceptable salt thereof, or a pharmaceutical composition of the present disclosure. In some embodiments, the method of the present disclosure includes degrading STAT6 protein in a subject in need thereof.

In some embodiments, the present disclosure provides a method of treating a disease or condition mediated by a signal transducer and activator of transcription 6 (STAT6) protein activity in a subject in need thereof, the method comprising administering to the subject a therapeutically effective amount of a compound of the present disclosure, or a pharmaceutically acceptable salt thereof, or a pharmaceutical composition of the present disclosure.

In some embodiments, the method of treating a disease or condition mediated by a signal transducer and activator of transcription 6 (STAT6) protein activity in a subject in need thereof is the method wherein the STAT6-mediated disorder or disease is from the class of diseases or rheumatology, gastroenterology, pulmonology, hepatology, nephrology, dermatology or an allergic disease.

In some embodiments, the method of treating a disease or condition mediated by a signal transducer and activator of transcription 6 (STAT6) protein activity in a subject in need thereof is the method wherein the STAT6-mediated disorder or disease is rheumatoid arthritis (RA), systemic lupus erythematosus (SLE), lupus nephritis (LN), osteoarthritis (OA), ulcerative colitis (UC), Crohn's disease (CD), idiopathic pulmonary fibrosis (IPF), interstitial lung disease (ILD), metabolic dysfunction-associated steatohepatitis (MASH), Diabetic kidney disease (DKD) (diabetic nephropathy), or atopic dermatitis (AD).

In some embodiments, the method of treating a disease or condition mediated by a signal transducer and activator of transcription 6 (STAT6) protein activity in a subject in need thereof is the method wherein the STAT6-mediated disorder or disease is atopic dermatitis, contact dermatitis, vitiligo, alopecia areata, acne, psoriasis, dermatomyositis, scleroderma, or morphea.

In some embodiments, the method of treating a disease or condition mediated by a signal transducer and activator of transcription 6 (STAT6) protein activity in a subject in need thereof is the method wherein the STAT6-mediated disorder or disease is atopic dermatitis.

In some embodiments, the present disclosure provides a method of modulating a signal transducer and activator of transcription 6 (STAT6) protein activity in a subject, wherein the method comprises administering to the subject a therapeutically effective amount of a compound of the present disclosure, or a pharmaceutically acceptable salt thereof, or a pharmaceutical composition of the present disclosure.

In some embodiments, the present disclosure provides a method of treating a disease or condition mediated by interleukin 4 (IL-4) or interleukin 3 (IL-3) in a subject in need thereof, the method comprising administering to the subject a therapeutically effective amount of a compound of the present disclosure, or a pharmaceutically acceptable salt thereof, or a pharmaceutical composition of the present disclosure.

In some embodiments, the present disclosure provides a method for manufacturing a medicament for treating a disease or condition mediated by a signal transducer and activator of transcription 6 (STAT6) protein activity in a subject in need thereof, characterized in that a compound of the present disclosure, or a pharmaceutically acceptable salt thereof, is used.

In some embodiments, the present disclosure provides use of the compound of the present disclosure, or a pharmaceutically acceptable salt thereof, for the manufacture of a medicament for the treatment of a disease or condition mediated by a signal transducer and activator of transcription 6 (STAT6) protein activity in a subject.

In some embodiments, the present disclosure provides the compound of the present disclosure, or a pharmaceutically acceptable salt thereof, for use in treating a disease or condition mediated by a signal transducer and activator of transcription 6 (STAT6) protein activity in a subject in need thereof.

In some embodiments, the present invention provides the compound of the present disclosure, or a pharmaceutically acceptable salt thereof, for use in therapy.

In some embodiments, the STAT6-associated disease or condition is an inflammation/immune disease or disorder. In some embodiments, the STAT6-associated disease or condition is a rheumatology disease, such as rheumatoid arthritis (RA), systemic lupus erythematosus (SLE), lupus nephritis (LN), or osteoarthritis (OA). In some embodiments, the STAT6-associated disease or condition is Sjogren's syndrome, systemic sclerosis (SSc), ankylosing spondylitis (AS), dermatomyositis, psoriatic arthritis (PsA), ANCA vasculitis, IgG4-related disease, non-radiographic axial spondyloarthritis (nr-AxSpA), polymyositis, or Takayasu arteritis. In some embodiments, the STAT6-associated disease or condition is cutaneous lupus erythematosus (CLE) types: chronic CLE (including discoid), subacute CLE, acute CLE; seropositive or seronegative RA, juvenile idiopathic arthritis (JIA); primary osteoarthritis or secondary osteoarthritis, cervical and lumbar spinal osteoarthritis, hip osteoarthritis, knee osteoarthritis, or erosive osteoarthritis.

In some embodiments, the STAT6-associated disease or condition is a gastroenterology disease, such as ulcerative colitis (UC), or Crohn's disease (CD). In some embodiments, the STAT6-associated disease or condition is eosinophilic gastrointestinal disorders (EGIDs), such as eosinophilic esophagitis (EoE), eosinophilic gastroenteritis, eosinophilic colitis, or microscopic colitis. In some embodiments, the STAT6-associated disease or condition is ulcerative proctitis, proctosignmoiditis, left-sided colitis, extensive colitis, pancolitis, ileocolitis, ileitis, gastroduodenal CD, jejunoileitis, or Crohn's (granulomatous) colitis.

In some embodiments, the STAT6-associated disease or condition is a pulmonology disease, such as idiopathic pulmonary fibrosis (IPF), or interstitial lung disease (ILD). In some embodiments, the STAT6-associated disease or condition is acute respiratory distress syndrome (ARDS), asthma: bronchiolitis obliterans, chronic obstructive pulmonary disease (COPD); Connective tissue disease-associated interstitial lung disease (CTD-ILD), collagen vascular disease, alveolar proteinosis, hypersensitivity pneumonitis (HP), non-cystic fibrosis bronchiectasis (non-CFB), cystic fibrosis; bronchiectasis; primary ciliary dyskinesia; pneumonia pulmonary arterial hypertension (PAH); lymphangioleiomyomatosis; nonspecific interstitial pneumonia; cryptogenic organizing pneumonia; acute interstitial pneumonia; familial interstitial lung disease, or bleomycin induced pulmonary fibrosis.

In some embodiments, the STAT6-associated disease or condition is a hepatology disease. In some embodiments, the STAT6-associated disease or condition is metabolic dysfunction-associated steatohepatitis (MASH). In some embodiments, the STAT6-associated disease or condition is Primary sclerosing cholangitis (PSC), primary biliary cirrhosis (PBC), autoimmune hepatitis, alcoholic steatohepatitis (ASH), or alcoholic hepatitis. In some embodiments, the STAT6-associated disease or condition is chronic intrahepatic or extrahepatic cholestatic disease, obstructive or chronic inflammatory disorders of the liver, liver fibrosis, liver cirrhosis, liver steatosis, liver ischemia, chemotherapy associated steatohepatitis (CASH), lipid and lipoprotein disorders, Type II Diabetes, Type I Diabetes, Non-Alcoholic Fatty Liver Disease (NAFLD), or Barrett's esophagus.

In some embodiments, the STAT6-associated disease or condition is a nephrology disease, such as diabetic kidney disease (DKD) (diabetic nephropathy). In some embodiments, the STAT6-associated disease or condition is chronic kidney disease (CKD), kidney disease, kidney fibrosis, kidney insufficiency, acute kidney injury, tubular disfunction, 2,8-dihydroxyadenine nephropathy, renal transplant rejection, renal protection against drugs inducing Fanconi's syndrome, hereditary fructose intolerance, metabolic syndrome, obesity, hyperlipidemia, hypertriglyceridemia, hypertension, steatosis, cardiometabolic syndrome, insulin resistance, cardiovascular disease, heart failure, type 1 and type 2 diabetes mellitus, or hyperuricemia.

In some embodiments, the STAT6-associated disease or condition is a dermatology or allergic disease, such as atopic dermatitis (AD). In some embodiments, the STAT6-associated disease or condition is atopic dermatitis, contact dermatitis, vitiligo, alopecia areata, acne, psoriasis, dermatomyositis, scleroderma, or morphea.

In some embodiments, the STAT6-associated disease or condition is an inflammation/immune disease or disorder, such as
adult-onset Still's disease, alcoholic hepatitis, alcoholic steatohepatitis, alcoholic liver disease, asthma, including allergen-induced asthma, bullous pemphigoid (BP) asthma, non-allergen induced asthma, allergies and allergic conditions such as allergic bronchopulmonary aspergillosis, allergic conjunctivitis, allergic encephalomyelitis, and allergic neuritis, food allergies, allograft rejection, alcoholic steatohepatitis (ASH), ANCA vasculitis, anti-glomerular basement membrane disease (Anti-GBM), antiphospholipid syndrome, aphthous stomatitis, appendicitis, arthritis, autoimmune diseases, atrophic thyroiditis, autoimmune hemolytic anemia (immune pancytopenia, paroxysmal nocturnal hemoglobinuria), autoimmune polyendocrinopathies, autoimmune thrombocytopenia (idiopathic thrombocytopenic purpura, immune-mediated thrombocytopenia), autoimmune hepatitis, pernicious anemia (Addison's disease), and autoimmune thyroid disorders, autoinflammatory diseases, autosomal dominant polycystic kidney disease (ADPKD), ankylosing spondylitis (AS), acute respiratory distress syndrome (ARDS),
Bechet's disease or syndrome, bee sting-induced inflammation, Blau syndrome, bursitis, Barrett's esophagus, bleomycin induced pulmonary fibrosis, bronchiolitis obliterans,
cardiac hypertrophy, gluten-sensitive enteropathy (Celiac disease), chemical irritant-induced inflammation, chorioretinitis, chronic atypical neutrophilic dermatosis with lipodystrophy and elevated temperature (CANDLE) syndrome, chronic obstructive pulmonary disease (COPD), chronic pancreatitis, chronic prostatitis, chronic recurrent multifocal osteomyelitis, cicatricial alopecia, colitis, complex regional pain syndrome, chronic intrahepatic or extrahepatic cholestatic disease, conjunctivitis, connective tissue disease, Connective tissue disease-associated interstitial lung disease (CTD-ILD), corneal ulcer, cryopyrin-associated periodic syndromes, cutaneous lupus erythematosus (CLE), cystic fibrosis,
deficiency of the interleukin-1 receptor antagonist (DIRA), deficiency of IL36R antagonist (DITRA), dermatitis, diabetic kidney disease (DKD) (diabetic nephropathy), diverticulitis, discoid lupus erythematosus, drug induced delayed type cutaneous allergic reactions,
encephalitis, esophagitis, eosinophilic gastrointestinal disorders (EGIDs), such as eosinophilic esophagitis (EoE), eosinophilic gastroenteritis, eosinophilic colitis,
familial cold urticarial, familial Mediterranean fever, fistulizing Crohn's disease,
giant cell arteritis, glomerulonephritis, gout, gouty arthritis, graft-versus-host disease (GVHD), granulomatous hepatitis, Guillain-Barre syndrome (GBS), Graves' disease,
Hashimoto's thyroiditis, Henoch-Schönlein purpura, hidradenitis suppurativa (HS), hyaline membrane disease, hyperactive inflammatory response, hypereosinophilic syndrome (HES), hyperimmunoglobulinemia D with recurrent fever (HIDS), hypersensitivity pneumonitis (HP),
immunoglobulin (IgA) nephropathies, IgG4-related disease, immune complex nephritis, immune thrombocytopenic purpura (ITP), inflammation, inflammation of the CNS, inflammatory bowel disease (IBD), inflammatory disease of the respiratory tract (upper or lower) such as inflammatory lung disease, bronchitis, sinusitis, inflammatory ischemic event such as stroke or cardiac arrest, inflammatory liver disease, inflammatory myopathy, inflammatory neuropathy, inflammatory pain, insect bite-induced inflammation, interstitial cystitis, iritis, irritant-induced inflammation,
juvenile arthritis, juvenile rheumatoid arthritis,
keratitis, kidney transplant rejection, kidney disease, kidney fibrosis, kidney insufficiency,
leukocyte adhesion deficiency, Loeffler's syndrome, lupus, lupus nephritis (LN), liver fibrosis, liver steatosis, liver ischemia, lipid and lipoprotein disorders,
mast cell activation syndrome, mastocytosis, meningitis, microscopic colitis, mixed connective tissue disease, morphea or morphea variants, Muckle-Wells syndrome (urticaria deafness amyloidosis), mucositis, myelitis, myocarditis, myositis, necrotizing enterocolitis, neonatal onset multisystem inflammatory disease (NOMID),
nasal polyps, neovascular glaucoma, neuritis, non-alcoholic fatty liver disease (NAFLD), metabolic dysfunction-associated steatohepatitis (MASH), non-radiographic axial spondyloarthritis (nr-AxSpA), non-cystic fibrosis bronchiectasis (non-CFB),
obstructive or chronic inflammatory disorders of the liver, ocular allergy, optic neuritis, organ transplant rejection, osteoarthritis (OA), otitis,
pancreatitis, pancolitis, pelvic inflammatory disease, pemphigus vulgaris (PV), bullous pemphigoid (BP), pericarditis, periodontitis, PFAPA (periodic fever, aphthous stomatitis, pharyngitis, adenitis), plant irritant-induced inflammation, pneumocystis infection, pneumonia, pneumonitis, poison ivy/urushiol oil-induced inflammation, polyarteritis nodosa, polychondritis, polycystic kidney disease (PCKD), polymyalgia rheumatic, polymyositis, pouchitis, proctitis, proctosignmoiditis, psoriatic arthritis (PsA), pulmonary arterial hypertension (PAH), pulmonary fibrosis, pyogenic sterile arthritis, pruritus,
reperfusion injury and transplant rejection, primary biliary cirrhosis (PBC), primary sclerosing cholangitis (PSC), Raynaud's syndrome, Reiter's disease, reactive arthritis, renal graft rejection, reperfusion injury, rheumatic carditis, rheumatic diseases, rheumatic fever, rheumatoid arthritis (RA), rhinitis, rhinitis psoriasis,
sarcoidosis, Schnitzler syndrome, scleritis, sclerosis, such as systemic sclerosis (SSc), seborrhea, sepsis, septic shock, Sjogren's syndrome, inflammatory skin diseases or conditions, such as acne, alopecia areata, atopic dermatitis, rosacea, eczema, dermatitis, dermatitis endotoxemia, dermatomyositis, stasis dermatitis, Stevens-Johnson syndrome (SJS), skin irritation, skin rash, skin sensitization (contact dermatitis or allergic contact dermatitis), scleroderma, psoriasis, psoriasis vulgaris, psoriatic arthritis, spinal stenosis, spondyloarthropathies, synovial inflammation, systemic inflammatory response syndrome (SIRS), systemic lupus erythematosus (SLE), systemic mast cell disease (SMCD), systemic vasculitis, systemic-onset juvenile idiopathic arthritis,
temporal arteritis, tendinitis, tenosynovitis, thyroditis, transplantation rejection, tubulointerstitial nephritis, tubular disfunction, Takayasu arteritis, toxic epidermal necrolysis,
urticaria, uterine fibroids, uveitis, uveoretinitis,
vasculitis, vasculitis (NHLBI), vitiligo, or
Wegener's granulomatosis.

In some embodiments, the STAT6-associated disease or condition is a disease or disorder, such as
acne, acid-induced lung injury, Addison's disease, adrenal hyperplasia, adrenocortical insufficiency, age-related macular degeneration, aging, alcoholic liver disease, Alzheimer's disease, angina pectoris, angiofibroma, anhidrotic ectodermal dysplasia, ascites, aspergillosis, atherosclerosis, atherosclerotic plaques, amyloidosis, amyotrophic lateral sclerosis (ALS), angioedema, acute myocardial infarction, antigen-antibody complex mediated diseases, alpha-1-antitrypsin deficiency,
back pain, Bacillus anthracis infection, Bell's palsy, berylliosis, bone pain, burns, bullous pemphigoid,
cancer, carpal tunnel syndrome, Castleman's disease, catabolic disorders, cataracts, cerebral aneurysm, complications of organ transplantation, corneal graft neovascularization, cryptococcosis, a non-malignant hyperproliferative disorder, a malignant hyperproliferative disorder, hepatocellular carcinoma, colon adenoma, polyposis, colon adenocarcinoma, breast cancer, pancreatic adenocarcinoma,
chronic heart failure, chronic lung disease of prematurity, cardiometabolic syndrome, cardiovascular disease, cutaneous T cell lymphoma,
diabetic macular edema, dyslipidemia,
endometriosis, endotoxemia, eosinophilic GI disease (EGID), eosinophilic esophagitis (EoE), eosinophilic pneumonias, epicondylitis, epidermolysis bullosa, erythema multiforme, erythroblastopenia,
familial amyloidotic polyneuropathy, fetal growth retardation, fibromyalgia,
glaucoma, glioblastoma, glomerular disease, gut diseases, growth plate injuries,
hair loss, herpes zoster and simplex, hypoplastic and other anemias, head injury, hepatitis A, B, C, D, and E, herpes, headache, hearing loss, heart disease, hemangioma, hemophilic joints, hereditary periodic fever syndrome, heritable disorders of connective tissue, Hodgkin's disease, Huntington's disease, hyperammonemia, hypercalcemia, hypercholesterolemia, hemolytic anemia, hepatitis, hip replacement, hypertropic bone formation, hypersensitivity pneumonia, hereditary fructose intolerance, hypertension, hyperuricemia,
idiopathic demyelinating polyneuropathy, infectious diseases including viral diseases such as AIDS (HIV infection), ichthyosis, incontinentia pigmenti (IP, Bloch-Siemens syndrome), idiopathic thrombocytopenic purpura, infectious mononucleosis, ischemia/reperfusion, insulin resistance,
joint replacement,
kidney injury caused by parasitic infections,
leptospirosis, lichen sclerosus (LS), lichen planus, Lambert-Eaton myasthenic syndrome, Lyme disease, liver failure, including acute liver failure,
muscle wasting, muscular dystrophy, Marfan syndrome (MFS), meningioma, mesothelioma, multiple organ injury syndrome, myasthenia gravis (MG), myelodysplastic syndrome, metabolic syndrome, multiple sclerosis,
nephrotic syndrome, neuropathological diseases, nuclear factor-kappa B essential modulator (NEMO) deficiency syndrome,
obesity, Osler-Weber syndrome, osteogenesis imperfecta, osteonecrosis, osteoporosis,
pachyonychia congenita, Paget's disease, Paget's disease of bone, Parkinson's disease, periodic fever, pertussis, primary pulmonary hypertension, pyoderma gangrenosum, pyogenic granuloma retrolental fibroplasias, peritoneal endometriosis, Prurigo nodularis, psychosocial stress diseases, pulmonary disease, pulmonary hypertension,
respiratory distress syndrome, renal disease, retinal disease, retrolental fibroplasia, renal transplant rejection, renal protection against drugs inducing Fanconi's syndrome, respiratory tract illness caused by respiratory syncytial virus, rhinosinusitis, radiation induced fibrosis.
sarcoidosis, severe pain, sleep apnea, scoliosis, sickle cell anemia, sports injuries, sprains and strains, sunburn, spinal cord injury, Sézary syndrome, silica-induced disease (Silicosis), subarachnoid hemorrhage,
tuberculosis, tumor necrosis factor (TNF) receptor associated periodic syndrome (TRAPS), thrombosis, traumatic brain injury, tissue transplant, complications from type 1 or type 2 diabetes, toxoplasmosis, thrombocytopenia, trachoma,
vascular restenosis, ventilator induced lung injury,
Whipple's disease, or
2,8-dihydroxyadenine nephropathy.

In some embodiments, provided herein is a method of inhibiting a signal transducer and activator of transcription 6 (STAT6) protein in a subject in need thereof, the method comprising administering to the subject a therapeutically effective amount of a compound of Formula J, I, or Ia, or a pharmaceutically acceptable salt thereof, or a pharmaceutical composition comprising a compound of Formula J, I, or Ia.

In some embodiments, provided herein is a method of treating inflammation in a subject in need thereof, the method comprising administering to the subject a therapeutically effective amount of a compound of Formula J, I, or Ia, or a pharmaceutically acceptable salt thereof, or a pharmaceutical composition comprising a compound of Formula J, I, or Ia.

When treating or preventing a STAT6 associated disease or condition for which compounds of the present disclosure are indicated, generally satisfactory results are obtained when the compounds of the present disclosure are administered at a daily dosage of from about 0.1 milligram to about 300 milligram per kilogram of animal body weight. In some embodiments, the compounds of the present disclosure are given as a single daily dose or in divided doses two to six times a day, or in sustained release form. For most large mammals, the total daily dosage is from about 1 milligram to about 1000 milligrams, or from about 1 milligram to about 50 milligrams. In the case of a 70 kg adult human, the total daily dose will generally be from about 0.1 milligrams to about 200 milligrams. This dosage regimen may be adjusted to provide the optimal therapeutic response. In some embodiments, the total daily dosage is from about 1 milligram to about 900 milligrams, about 1 milligram to about 800 milligrams, about 1 milligram to about 700 milligrams, about 1 milligram to about 600 milligrams, about 1 milligram to about 400 milligrams, about 1 milligram to about 300 milligrams, about 1 milligram to about 200 milligrams, about 1 milligram to about 100 milligrams, about 1 milligram to about 50 milligrams, about 1 milligram to about 20 milligram, or about 1 milligram to about 10 milligrams.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are frequently alternated with resting periods of about 1 to 28 days, commonly about 7 days or about 14 days, between cycles. The treatment cycles, in other embodiments, may also be continuous.

In some embodiments, the methods provided herein comprise administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

In some embodiments, the compound or pharmaceutically acceptable salt thereof of the present disclosure is administered in combination with one or more additional therapeutic agent or therapeutic modality.

In some embodiments, the present disclosure provides the pharmaceutical composition or the method wherein the one or more additional therapeutic agent or therapeutic modality comprises one, two, three, or four additional therapeutic agents and/or therapeutic modalities.

In some embodiments, the method includes administering one or more additional therapeutic agents. The one or more additional therapeutic agents can be one or more therapeutic agents as described below.

In another embodiment, the present disclosure provides a method for manufacturing a medicament for treating inflammation in a subject in need thereof, characterized in that a compound of the present disclosure, or a pharmaceutically acceptable salt thereof, is used.

In another embodiment, the present disclosure provides use of the compound of the present disclosure, or a pharmaceutically acceptable salt thereof, for the manufacture of a medicament for the treatment of inflammation in a subject.

In another embodiment, the present disclosure provides the compound of the present disclosure, or a pharmaceutically acceptable salt thereof, for use in the treatment of inflammation in a subject in need thereof.

In another embodiment, the present disclosure provides the compound of the present disclosure, or a pharmaceutically acceptable salt thereof, for use in therapy.

### VIII. Combination Therapy

In some embodiments, a compound of Formula J, I, or Ia, provided herein, or pharmaceutically acceptable salt thereof, is administered in combination with one or more additional therapeutic agents to treat or prevent a disease or condition disclosed herein. In some embodiments, the one or more additional therapeutic agents are one, two, three, or four additional therapeutic agents. In some embodiments, the one or more additional therapeutic agents are one additional therapeutic agent. In some embodiments, the one or more additional therapeutic agents are two additional therapeutic agents. In some embodiments, the one or more additional therapeutic agents are three additional therapeutic agents. In some embodiments, the one or more additional therapeutic agents are four additional therapeutic agents.

In some embodiments, the pharmaceutical compositions provided herein include a compound of Formula J, I, or Ia provided herein, or pharmaceutically acceptable salt thereof, and one or more additional therapeutic agents. In some embodiments, the one or more additional therapeutic agents are one, two, three, or four additional therapeutic agents. In some embodiments, the one or more additional therapeutic agents are one additional therapeutic agent. In some embodiments, the one or more additional therapeutic agents are two additional therapeutic agents. In some embodiments, the one or more additional therapeutic agents are three additional therapeutic agents. In some embodiments, the one or more additional therapeutic agents are four additional therapeutic agents.

In some embodiments, a compound of the disclosure is co-administered with one or more (e.g., one, two, three, or four) additional therapeutic agents. In some embodiments, the additional therapeutic agent includes an agent useful for modulating, treating, or preventing inflammation, such as a 4-1BB ligand, 5-Alpha-reductase inhibitor, 5-HT 1a receptor antagonist, 5-HT 1a receptor partial agonist, 5-HT 2a receptor antagonist, 5-HT 2a receptor partial agonist, 5-HT 2b receptor antagonist, 5-HT 3 receptor antagonist, 5-HT 4 receptor agonist, 5-HT 6 receptor antagonist, 5-HT 7 receptor antagonist, 5-Lipoxygenase activating protein inhibitor, 5-Lipoxygenase inhibitor, Accessory gene regulator A inhibitor, Acetaldehyde dehydrogenase modulator, Acetylcholine receptor agonist, Acetylcholine receptor antagonist, Acetylcholinesterase inhibitor, Acidic mammalian chitinase inhibitor, ACTH receptor agonist, Activity-dependent neuroprotector modulator, ADAM-33 inhibitor, ADAM-9 inhibitor, Adenosine A1 receptor antagonist, Adenosine A1 receptor modulator, Adenosine A2b receptor antagonist, Adenosine A3 receptor agonist, Adenosine A3 receptor antagonist, Adenosine deaminase stimulator, Adenosylhomocysteinase inhibitor, Adenylate cyclase stimulator, Adrenergic receptor agonist, Adrenocorticotrophic hormone ligand, Advanced glycosylation product receptor antagonist, AGER gene inhibitor, AIMP multisynthetase complex protein 1 inhibitor, Albumin antagonist, Alcohol dehydrogenase 5 inhibitor, Aldose reductase inhibitor, Alk-5 protein kinase inhibitor, Alpha 1 antitrypsin modulator, Alpha 1 antitrypsin stimulator, Alpha 1 proteinase inhibitor, Alpha 2 adrenoceptor agonist, Amiloride sensitive sodium channel inhibitor, AMP activated protein kinase alpha 2 stimulator, AMP activated protein kinase stimulator, Amyloid protein deposition inhibitor, Androgen receptor antagonist, Angiotensin II AT-1 receptor antagonist, Angiotensin II AT-2 receptor agonist, Anoctamin 1 stimulator, Aortic smooth muscle actin inhibitor, AP1 transcription factor modulator, Apelin receptor agonist, Apolipoprotein A antagonist, Apolipoprotein A5 stimulator, Apolipoprotein B modulator, Apolipoprotein E modulator, Apoptosis regulator Bcl X inhibitor, Apoptosis regulator Bcl w inhibitor, APRIL receptor modulator, Aryl hydrocarbon receptor agonist, Aryl hydrocarbon receptor modulator, B and T lymphocyte attenuator stimulator, B-lymphocyte antigen CD19 inhibitor, B-lymphocyte antigen CD19 modulator, B-lymphocyte antigen CD20 inhibitor, B-lymphocyte stimulator ligand inhibitor, B-lymphocyte stimulator ligand modulator, Bcl-2 protein inhibitor, Beta 1 adrenoceptor antagonist, Beta 2 adrenoceptor agonist, Beta 2 adrenoceptor antagonist, Beta 2 adrenoceptor modulator, Beta adrenoceptor agonist, Beta amyloid antagonist, Beta-catenin inhibitor, Beta-catenin modulator, Bifunctional aminoacyl tRNA synthetase inhibitor, BMP10 gene inhibitor, BMP15 gene inhibitor, Bone marrow proteoglycan modulator, Botulinum toxin A stimulator, Bromodomain containing protein 1 inhibitor, Bromodomain containing protein inhibitor, Btk tyrosine kinase inhibitor, C-myc binding protein inhibitor, C-type lectin domain protein 4C inhibitor, Ca2+ release activated Ca2+ channel 1 inhibitor, Calcineurin inhibitor, Calcium channel inhibitor, Cannabinoid CB1 receptor antagonist, Cannabinoid CB1 receptor inverse agonist, Cannabinoid CB2 receptor agonist, Cannabinoid CB2 receptor modulator, Cannabinoid receptor agonist, Cannabinoid receptor antagonist, Cannabinoid receptor modulator, Catalase stimulator, CCL26 gene inhibitor, CCR3 chemokine modulator, CCR5 chemokine antagonist, CCR6 chemokine antagonist, CCR8 chemokine antagonist, CD11b antagonist, CD122 agonist, CD122 modulator, CD19 modulator, CD2 antagonist, CD223 modulator, CD3 modulator, CD30 modulator, CD4 antagonist, CD40 ligand receptor antagonist, CD47 antagonist, CDw123 modulator, Cell adhesion molecule inhibitor, Cell surface glycoprotein CD200R agonist, Cell surface glycoprotein MUC18 inhibitor, CFTR modulator, CFTR stimulator, Chaperonin stimulator, Chemokine receptor antagonist, Chitinase inhibitor, Collagen I agonist, Collagen I antagonist, Collagen modulator, Collagen VII antagonist, Complement C1q subcomponent inhibitor, Complement C1s subcomponent inhibitor, Complement C5 factor inhibitor, Complement factor C2 inhibitor, Complement factor D inhibitor, COVID19 spike glycoprotein modulator, CSF-1 antagonist, CXC10 chemokine ligand inhibitor, CXCR2 chemokine antagonist, cyclic GMP AMP synthase inhibitor, Cyclooxygenase inhibitor, Cytokine receptor agonist, Cytokine receptor antagonist, Cytokine receptor common beta chain inhibitor, Cytoplasmic protein NCK inhibitor, Cytosolic phospholipase A2 inhibitor, Cytotoxic T-lymphocyte protein-4 stimulator, Deoxyribonuclease gamma stimulator, DHFR inhibitor, Diacylglycerol O acyltransferase 1 inhibitor, Dihydroorotate dehydrogenase inhibitor, Dipeptidyl peptidase I inhibitor, Dipeptidyl peptidase IV inhibitor, DNA gyrase inhibitor, DNA methyltransferase inhibitor, Dopamine D2 receptor partial agonist, Dopamine D3 receptor agonist, Dopamine D3 receptor partial agonist, Dopamine D4 receptor partial agonist, DYRK-1 alpha protein kinase inhibitor, Ectonucleotide pyrophosphatase-PDE-2 inhibitor, EGF like module receptor 1 antagonist, EGFR family tyrosine kinase receptor inhibitor, Elastase inhibitor, Endonuclease modulator, Endostatin modulator, Endothelin ET-A receptor antagonist, Endothelin ET-B receptor antagonist, Enolase 1 inhibitor, Eosinophil peroxidase inhibitor, Eotaxin 2 ligand inhibitor, Eotaxin ligand inhibitor, EP4 prostanoid receptor antagonist, Epidermal growth factor receptor antagonist, Epidermal growth factor receptor modulator, Estradiol agonist, Estrogen receptor agonist, Extracellular signal related kinase-2 inhibitor, Facilitated glucose transporter-1 modulator, Fatty acid synthase inhibitor, FGF receptor antagonist, FGF-2 ligand, FGF-4 ligand, FGF3 receptor antagonist, Filaggrin stimulator, Flt3 tyrosine kinase inhibitor, FMLP related receptor I agonist, FMLP related receptor II agonist, Free fatty acid receptor 2 agonist, Free fatty acid receptor 3 agonist, Fyn tyrosine kinase inhibitor, FXR agonist, G-protein coupled bile acid receptor 1 agonist, G-protein coupled receptor-44 antagonist, G-protein coupled receptor-44 modulator, GABA A receptor agonist, GABA A receptor alpha-2 subunit modulator, GABA A receptor alpha-3 subunit modulator, GABA A receptor alpha-5 subunit modulator, Galectin-10 modulator, GATA 3 transcription factor inhibitor, Glucagon-like peptide 1 receptor agonist, Glucocorticoid receptor agonist, Glutamate dehydrogenase modulator, Glutamate receptor modulator, Glutathione dependent PGD synthase inhibitor, Glutathione independent PGD synthase inhibitor, Glutathione reductase inhibitor, GroEL protein 2 inhibitor, Guanylate cyclase stimulator, H+ K+ ATPase inhibitor, Heat shock protein inhibitor, Heme oxygenase 1 modulator, Heparin agonist, High mobility group protein B1 inhibitor, Histamine H1 receptor antagonist, Histamine H4 receptor antagonist, Histamine receptor antagonist, Histone deacetylase-1 inhibitor, Histone deacetylase-2 inhibitor, Histone deacetylase-2 stimulator, Histone deacetylase-3 inhibitor, Histone deacetylase-6 inhibitor, Histone H2A modulator, Histone H4 modulator, HMG CoA reductase inhibitor, Hsp 90 inhibitor, Hsp70 binding protein 1 inhibitor, Hyaluronidase stimulator, Hypoxia inducible factor stimulator, I-kappa B kinase beta inhibitor, I-kappa B kinase epsilon inhibitor, IgG receptor FcRn large subunit p51 modulator, IL-1 receptor accessory protein inhibitor, IL-1 receptor antagonist, IL-10 receptor agonist, IL-10 receptor antagonist, IL-12 receptor antagonist, IL-13 receptor antagonist, IL-13 receptor modulator, IL-15 receptor antagonist, IL-17 antagonist, IL-18 antagonist, IL-2 receptor alpha subunit stimulator, IL-2 receptor antagonist, IL-2 receptor modulator, IL-22 antagonist, IL-23 antagonist, IL-3 receptor modulator, IL-4 receptor antagonist, IL-4 receptor modulator, IL-5 receptor antagonist, IL-6 receptor antagonist, IL-7 receptor antagonist, IL-8 receptor antagonist, IL17RA gene inhibitor, IL2 gene stimulator, Immunoglobulin E antagonist, Immunoglobulin E modulator, Immunoglobulin G agonist, Immunoglobulin G1 modulator, Immunoglobulin agonist, Immunoglobulin kappa modulator, Immunoglobulin modulator, Inducible nitric oxide synthase inhibitor, Insulin receptor substrate-1 inhibitor, Insulin-like growth factor 1 receptor antagonist, Integrin alpha-2/beta-1 antagonist, Integrin alpha-4/beta-1 antagonist, Integrin alpha-4/beta-7 antagonist, Integrin alpha-5/beta-1 antagonist, Integrin alpha-5/beta-3 modulator, Integrin alpha-V/beta-1 antagonist, Integrin beta 1 binding protein modulator, Interferon alpha 14 ligand, Interferon alpha ligand inhibitor, Interferon beta ligand, Interferon beta ligand inhibitor, Interferon gamma ligand inhibitor, Interferon gamma receptor antagonist, Interferon type I receptor antagonist, Interleukin 1 delta ligand inhibitor, Interleukin 1 like receptor (IL33R) antagonist, Interleukin 1 like receptor 1 modulator, Interleukin 1 like receptor 2 inhibitor, Interleukin 13 ligand inhibitor, Interleukin 13 receptor alpha 1 antagonist, Interleukin 15 ligand inhibitor, Interleukin 17 ligand inhibitor, Interleukin 17A ligand inhibitor, Interleukin 17A ligand modulator, Interleukin 17E ligand inhibitor, Interleukin 17E ligand modulator, Interleukin 17F ligand inhibitor, Interleukin 17F ligand modulator, Interleukin 18 ligand inhibitor, Interleukin 23A inhibitor, Interleukin 31 ligand inhibitor, Interleukin 31 ligand modulator, Interleukin 33 ligand inhibitor, Interleukin 33 ligand modulator, Interleukin receptor 17A antagonist, Interleukin receptor 17B antagonist, Interleukin-1 alpha ligand inhibitor, Interleukin-1 beta ligand modulator, Interleukin-1 ligand inhibitor, Interleukin-2 ligand, Interleukin-2 ligand inhibitor, Interleukin-31 receptor modulator, Interleukin-4 ligand inhibitor, Interleukin-5 ligand inhibitor, Interleukin-6 ligand inhibitor, Interleukin-8 ligand inhibitor, Interleukin-9 ligand inhibitor, IRAK-4 protein kinase inhibitor, IRAK-4 protein kinase degrader, Itk tyrosine kinase inhibitor, JAK tyrosine kinase inhibitor, Jak1 tyrosine kinase inhibitor, Jak2 tyrosine kinase inhibitor, Jak3 tyrosine kinase inhibitor, Jun N terminal kinase inhibitor, Kallikrein 2 inhibitor, Kallikrein 5 modulator, Kallikrein 7 inhibitor, Kallikrein 7 modulator, Kallikrein inhibitor, KCNA voltage-gated potassium channel-3 inhibitor, Kelch like ECH associated protein 1 inhibitor, Kelch like ECH associated protein 1 modulator, Kit tyrosine kinase inhibitor, LanC like protein 2 stimulator, Lanosterol-14 demethylase inhibitor, Lck tyrosine kinase inhibitor, Lectin mannose binding protein inhibitor, Leukocyte Ig like receptor A4 modulator, Leukocyte elastase inhibitor, Leukotriene A4 hydrolase inhibitor, Leukotriene BLT receptor antagonist, Leukotriene C4 antagonist, Leukotriene C4 synthase inhibitor, Leukotriene D4 agonist, Leukotriene D4 antagonist, Leukotriene E4 antagonist, Leukotriene receptor antagonist, Liver X receptor agonist, LOXL2 gene inhibitor, Lung surfactant associated protein D stimulator, Lyn tyrosine kinase inhibitor, Lysophosphatidate-1 receptor antagonist, Lysophospholipase inhibitor, Macrophage migration inhibitory factor inhibitor, Major allergen I polypeptide chain 2 inhibitor, Major allergen inhibitor, MALT protein 1 inhibitor, Mannan-binding lectin serine protease inhibitor, MAP kinase modulator, MAPKAPK2 inhibitor, MARCKS protein inhibitor, Mas-related G-protein receptor X2 antagonist, Mas-related G-protein receptor X2 inhibitor, MEK protein kinase inhibitor, MEK-1 protein kinase inhibitor, Melanocortin MC1 receptor agonist, Melanocortin MC5 receptor antagonist, Melanocortin receptor agonist, Melanocyte stimulating hormone ligand, Membrane copper amine oxidase inhibitor, Metalloprotease-12 inhibitor, MEX3B gene inhibitor, Mineralocorticoid receptor antagonist, MIP 3 alpha ligand inhibitor, Mite allergen modulator, Mitochondrial 10 kDa heat shock protein stimulator, MKL myocardin like protein inhibitor, MMP1 gene stimulator, MNK protein kinase inhibitor, Monocyte chemotactic protein 1 ligand inhibitor, MS4A2 gene modulator, mTOR complex 1 inhibitor, mTOR complex 2 inhibitor, MUC5AC gene inhibitor, Muscarinic M1 receptor antagonist, Muscarinic M2 receptor antagonist, Muscarinic M3 receptor antagonist, Muscarinic M4 receptor antagonist, Muscarinic M5 receptor antagonist, Muscarinic receptor agonist, Muscarinic receptor antagonist, Muscarinic receptor modulator, Myeloperoxidase inhibitor, Myosin heavy chain inhibitor, NACHT LRR PYD domain protein 3 inhibitor, NEDD4 family interacting protein 1 stimulator, Neuropilin 2 modulator, Neutral endopeptidase inhibitor, NFE2L2 gene stimulator, Nicotinic aCh receptor alpha 7 subunit stimulator, Nicotinic acetylcholine receptor agonist, NK1 receptor antagonist, NKG2 D activating NK receptor antagonist, NLR family member X1 stimulator, Non receptor tyrosine kinase TYK2 antagonist, Nuclear erythroid 2-related factor 1 stimulator, Nuclear erythroid 2-related factor 2 stimulator, Nuclear erythroid 2-related factor inhibitor, Nuclear factor kappa B gene inhibitor, Nuclear factor kappa B inducing kinase inhibitor, Nuclear factor kappa B inhibitor, Nuclear factor kappa B modulator, Oncostatin M receptor subunit beta inhibitor, Opioid growth factor receptor agonist, Opioid receptor delta antagonist, Opioid receptor kappa agonist, Orexin 1 receptor antagonist, Orexin 2 receptor antagonist, Orphan nuclear receptor antagonist, Outer membrane protein modulator, OX-40 receptor agonist, OX-40 receptor antagonist, OX40 ligand inhibitor, OX40 ligand modulator, Oxoeicosanoid receptor 1 antagonist, P-Glycoprotein inhibitor, P-selectin glycoprotein ligand-1 inhibitor, P2X2 purinoceptor antagonist, P2X3 purinoceptor antagonist, P2X7 purinoceptor modulator, P2Y6 purinoceptor modulator, p38 MAP kinase inhibitor, p53 tumor suppressor protein stimulator, PcrV protein type III inhibitor, PDE 3 inhibitor, PDE 4 inhibitor, PDE 4b inhibitor, PDE 4d inhibitor, PDE 5 inhibitor, PDGF receptor antagonist, Peptidase 1 inhibitor, PGD2 antagonist, PGI2 agonist, Phosphatidylinositol 4 kinase beta inhibitor, Phosphoinositide 3-kinase inhibitor, Phosphoinositide-3 kinase delta inhibitor, Phospholipase A2 inhibitor, Phospholipase C inhibitor, PIM-1 protein kinase inhibitor, PIM-2 protein kinase inhibitor, PIM-3 protein kinase inhibitor, Placenta growth factor ligand inhibitor, Plasminogen activator inhibitor 1 inhibitor, Platelet activating factor receptor antagonist, Poly ADP ribose polymerase 14 inhibitor, PPAR gamma agonist, PPAR gene modulator, Progesterone receptor agonist, Programmed cell death ligand 1 modulator, Programmed cell death protein 1 modulator, Programmed cell death protein 1 stimulator, Prostaglandin E synthase inhibitor, Prostaglandin E synthase-1 inhibitor, Protease inhibitor, Protease-activated receptor-2 antagonist, Proteasome beta-8 subunit modulator, Proteasome inhibitor, Protein kinase C theta inhibitor, Protein kinase inhibitor, Protein NOV homolog modulator, Protein tyrosine kinase inhibitor, Protoporphyrinogen oxidase modulator, Pyruvate kinase muscle isozyme stimulator, Raf B protein kinase inhibitor, Ras gene inhibitor, Reactive oxygen species modulator inhibitor, Ret tyrosine kinase receptor inhibitor, Retinoic acid receptor agonist, Retinoic acid receptor antagonist, Retinoic acid receptor gamma agonist, Retinoic acid receptor gamma antagonist, Retinoic acid receptor gamma inverse agonist, Retinoic acid receptor modulator, Retinoid receptor agonist, Retinoid X receptor agonist, Retinoid X receptor modulator, Retinoid Z receptor gamma antagonist, Retinoid Z receptor gamma inverse agonist, Rev protein modulator, Rho associated protein kinase 1 inhibitor, Rho associated protein kinase 2 inhibitor, Ribonuclease P inhibitor, Ribonuclease stimulator, RIP-1 kinase inhibitor, S100 calcium binding protein A4 inhibitor, S100A8 gene inhibitor, S100A9 gene inhibitor, SARS coronavirus 3C protease like inhibitor, Secretory phospholipase A2 receptor antagonist, Serine protease inhibitor, Serine threonine protein kinase TBK1 inhibitor, Serum amyloid A protein modulator, SH2 domain containing protein inhibitor, Sialic acid-binding Ig-like lectin 8 inhibitor, SIRT3 gene stimulator, SMAD inhibitor, SNAI1 transcription factor inhibitor, SOD3 gene stimulator, Sodium channel inhibitor, Sphingosine 1 phosphate phosphatase 1 stimulator, Sphingosine-1-phosphate receptor-1 agonist, Sphingosine-1-phosphate receptor-1 antagonist, Sphingosine-1-phosphate receptor-1 modulator, Sphingosine-1-phosphate receptor-3 modulator, Sphingosine-1-phosphate receptor-4 antagonist, Sphingosine-1-phosphate receptor-4 modulator, Sphingosine-1-phosphate receptor-5 modulator, Sphingosylphosphorylcholine receptor antagonist, Src tyrosine kinase inhibitor, STAT inhibitor, STAT-1 modulator, STAT-3 inhibitor, STAT-5 inhibitor, STAT-6 inhibitor, STAT-6 degrader, Stearoyl CoA desaturase-1 inhibitor, Stress induced secreted protein 1 stimulator, Superoxide dismutase modulator, Superoxide dismutase stimulator, Syk tyrosine kinase inhibitor, Synuclein alpha inhibitor, T cell receptor antagonist, T cell receptor modulator, T cell surface glycoprotein CD28 inhibitor, T-cell antigen CD7 modulator, T-cell differentiation antigen CD6 inhibitor, T-cell surface glycoprotein CD8 inhibitor, T-cell transcription factor NFAT modulator, Tau aggregation inhibitor, Tau deposition inhibitor, Tec tyrosine kinase inhibitor, TGF beta 1 ligand inhibitor, TGF beta 3 ligand inhibitor, TGF beta 3 ligand modulator, TGF beta ligand inhibitor, TGF beta receptor agonist, TGF beta receptor antagonist, TGF-beta activated kinase-1 inhibitor, TGF-beta type II receptor antagonist, TGF-beta type II receptor modulator, TGF-beta type III receptor antagonist, Thromboxane A2 antagonist, Thromboxane synthetase inhibitor, Thymic stromal lymphopoietin ligand, Thymic stromal lymphopoietin ligand inhibitor, Thymic stromal lymphopoietin ligand modulator, Thymic stromal lymphopoietin receptor antagonist, Thymic stromal lymphopoietin receptor modulator, TLR agonist, TLR modulator, TLR-2 agonist, TLR-2 antagonist, TLR-4 antagonist, TLR-6 agonist, TLR-7 antagonist, TLR-8 antagonist, TLR-9 agonist, TLR-9 antagonist, TNF agonist, TNF alpha ligand inhibitor, TNF alpha ligand modulator, TNF antagonist, TNF binding agent, TNF related apoptosis inducing ligand, TNF-like receptor-2 modulator, Tumor necrosis factor 15 ligand inhibitor, Topoisomerase IV inhibitor, TRAIL receptor agonist, Transcription factor inhibitor, Transcription factor modulator, Transthyretin modulator, Trk tyrosine kinase receptor inhibitor, TRP cation channel A1 inhibitor, TRP cation channel A1 modulator, TRP cation channel C1 inhibitor, TRP cation channel V1 antagonist, TRP cation channel V1 modulator, TRP cation channel V2 modulator, Ts1 protein kinase inhibitor, Tubulin binding agent, Tubulin receptor antagonist, Tumor necrosis factor 13B receptor modulator, Tumor necrosis factor 13C receptor modulator, Tumor necrosis factor 14 ligand inhibitor, Tumor necrosis factor 15 ligand modulator, Tumor necrosis factor ligand inhibitor, Txk tyrosine kinase inhibitor, Tyk2 tyrosine kinase inhibitor, Tyk2 tyrosine kinase modulator, Type I IL-1 receptor antagonist, Type I TNF receptor antagonist, Type II TNF receptor modulator, Tyrosine phosphatase substrate 1 inhibitor, Ubiquitin inhibitor, Ubiquitin ligase modulator, Ubiquitin ligase stimulator, Ubiquitin thioesterase-4 inhibitor, Unspecified GPCR antagonist, Unspecified GPCR modulator, Unspecified ion channel inhibitor, Uteroglobin stimulator, V-set transmembrane domain protein 1 stimulator, Vascular cell adhesion protein 1 antagonist, VEGF ligand inhibitor, VEGF receptor modulator, VEGF-2 receptor modulator, Vimentin inhibitor, Vitamin D3 receptor agonist, Wnt 5A ligand inhibitor, Wnt ligand modulator or YSK-4 protein kinase inhibitor.

In some embodiments, a compound of the disclosure, or a pharmaceutically acceptable salt thereof, is co-administered with one or more agents useful for the treatment and/or prophylaxis of a dermatologic or allergic condition, such as atopic dermatitis (AD). Non-limiting examples of such agents include topical corticosteroids (TCS) (e.g., desonid, hydrocortisone, fluocinolone, triamcinolone, betamethasone diproprionate), topical calcineurin inhibitors (TCI) (e.g., tacrolimus, pimecrolimus), cyclosporine, methotrexate, mycophenolate mofetil, azathioprine, interferon gamma, phosphodiesterase 4 (PDE4) inhibitor such as crisaborole, JAK inhibitor (e.g., ruxolitinib, upadacitinib, abrocitinib, baricitinib), systemic glucocorticoids (e.g., prednisone), dupilumab, and anti-IL-13 antibody (e.g., tralokinumab).

In some embodiments, a compound of the disclosure, or a pharmaceutically acceptable salt thereof, is co-administered with one or more agents useful for the treatment and/or prophylaxis of a dermatologic or allergic condition, such as acne. Non-limiting examples of such agents include topical therapies such as benzoyl peroxide, topical retinoids, topical antibiotic, clascoterone, salicylic acid and azelaic acid; and systemic therapies such as doxycycline, minocycline, sarecycline, combined oral contraceptives, spironolactone, and isotretinoin.

In some embodiments, a compound of the disclosure, or a pharmaceutically acceptable salt thereof, is co-administered with one or more agents useful for the treatment and/or prophylaxis of a dermatologic or allergic condition, such as alopecia areata. Non-limiting examples of such agents include topical therapies such as systemic corticosteroids (such as prednisolone), cyclosporine, azathioprine, methotrexate, sulfasalazine, simvastatin/exetimibe, inosiplex, antihistamines (such as fexofenadine), and oral JAK inhibitors (such as ritlecitinib or brepocitinib).

In some embodiments, a compound of the disclosure, or a pharmaceutically acceptable salt thereof, is co-administered with one or more agents useful for the treatment and/or prophylaxis of a dermatologic or allergic condition, such as asthma. Non-limiting examples of such agents include inhaled ICS-formoterol (such as budesonide-formoterol), short-acting beta2 agonists (such as albuterol sulfate), leukotriene receptor antagonists (such as montelukast), immunoglobulin E antibodies (such as omalizumab) and long-acting muscarinic antagonists (such as tiotropium).

In some embodiments, a compound of the disclosure, or a pharmaceutically acceptable salt thereof, is co-administered with one or more agents useful for the treatment and/or prophylaxis of a dermatologic or allergic condition, such as chronic obstructive pulmonary disease (COPD). Non-limiting examples of such agents include short-acting beta2 agonists (such as albuterol sulfate), short-acting muscarinic antagonists (such as aiprtropium), long-acting beta2 agonists (such as olodaterol), and long-acting muscarinic antagonists (such as tiotropium).

In some embodiments, a compound of the disclosure, or a pharmaceutically acceptable salt thereof, is co-administered with one or more agents useful for the treatment and/or prophylaxis of a dermatologic or allergic condition, such as chronic rhinosinusitis with nasal polyps. Non-limiting examples of such agents include intranasal corticosteroid, or biologics such as benralizumab (targets IL-5), dupilumab (targets IL-13), omalizumab (targets IgE).

In some embodiments, a compound of the disclosure, or a pharmaceutically acceptable salt thereof, is co-administered with one or more agents useful for the treatment and/or prophylaxis of a dermatologic or allergic condition, such as contact dermatitis. Non-limiting examples of such agents include topical corticosteroids, topical calcineurin inhibitors (such as pimecrolimus, tacrolimus), topical phosphodiesterase 4 inhibitors, such as crisaborole, systemic immunosuppressants and modulators, such as systemic corticosteroids, methotrexate, azathioprine, mycophenolate mofetil, cyclosporine or dupilumab.

In some embodiments, a compound of the disclosure, or a pharmaceutically acceptable salt thereof, is co-administered with one or more agents useful for the treatment and/or prophylaxis of a dermatologic or allergic condition, such as dermatomyositis. Non-limiting examples of such agents include topical corticosteroids, topical calcineurin inhibitors (such as pimecrolimus, tacrolimus), systemic corticosteroids (such as prednisone), antimalarials (such as hydroxychloroquine, cholorquine, quinacrine), methotrexate, mycophenolate mofetil, intravenous immunoglobulin, rituximab, and JAK inhibitors (such as tofacitinib).

In some embodiments, a compound of the disclosure, or a pharmaceutically acceptable salt thereof, is co-administered with one or more agents useful for the treatment and/or prophylaxis of a dermatologic or allergic condition, such as esophageal eosinophilia. Non-limiting examples of such agents include systemic corticosteroids (such as budesonide, fluticasone, prednisone), topical corticosteroids, and proton pump inhibitors (such as omeprazole, esomeprazole, pantoprazole and lansoprazole).

In some embodiments, a compound of the disclosure, or a pharmaceutically acceptable salt thereof, is co-administered with one or more agents useful for the treatment and/or prophylaxis of a dermatologic or allergic condition, such as psoriasis. Non-limiting examples of such agents include topical treatments such as topical corticosteroids (such as betamethasone dipropionate, clobetasol propionate, desoximetasone, diflorasone diacetate, fluocinonide, flurandrenolide, halobetasol propionate, amcinonide, mometasone furoate, triamcinolone acetonide, fluticasone propionate, hydrocortisone valerate, clocortolone pivalate), topical calcineurin inhibitors (such as pimecrolimus, tacrolimus), topical vitamin D analogues (such as calcipotriene, calcitriol, tacalcitol or mazacalcitol), topical retinoids (such as tazarotene); systemic nonbiologic therapies such as methotrexate, phosphodiesterase 4 inhibitors (such as apremilast), immunosuppressants (such as cyclosporine), oral retinoids (such as acitretin), oral Janus kinase inhibitors (such as tofacitinib), fumaric acid esters (such as dimethyl fumarate), systemic immunosuppressants and antimetabolites (such as hydroxyurea, mycophenolate mofetil, azathioprine, leflunomide, tacrolimus and thioguanine); and biologic therapies such as TNF-α inhibitors (such as etanercept, infliximab, adalimumab, certolizumab), IL-12/IL-23 inhibitors (such as ustekinumab), IL-17 inhibitors (such as secukinumab, ixekizumab, brodalumab), and IL-23 inhibitors (such as guselkumab, tildrakizumab, risankizumab).

In some embodiments, a compound of the disclosure, or a pharmaceutically acceptable salt thereof, is co-administered with one or more agents useful for the treatment and/or prophylaxis of a dermatologic or allergic condition, such as scleroderma. Non-limiting examples of such agents include immunosuppressive treatments (such as methotrexate, mycophenolate mofetil, cyclophosphamide, tocilizumab, and rituximab), and autologous haematopoietic stem cell transplantation.

In some embodiments, a compound of the disclosure, or a pharmaceutically acceptable salt thereof, is co-administered with one or more agents useful for the treatment and/or prophylaxis of a dermatologic or allergic condition, such as vitiligo. Non-limiting examples of such agents include topical treatments such as topical corticosteroids, topical calcineurin inhibitors (such as pimecrolimus, tacrolimus), topical vitamin D analogues (such as calcipotriene); and systemic therapies such as oral corticosteroids (such as betamethasone).

In some embodiments, an additional therapeutic agent includes one or more of 608, 610, 611, clindamycin phosphate+benzoyl peroxide, 101BHG-D01, 1-H-11, 4P-022, 5-OXO-ETE receptor antagonists, 9MW-1911, AB-1000, AB-101a, abatacept, ABBV-712, ABCL-575, AbIPL-IL-17, ABM-125, abrocitinib, ABY-035/AFO2, ABY-062, AC-201, ACE-1334, acitretin, aclidinium bromide, aclidinium bromide+formoterol fumarate, acumapimod, AD-17002, adakitug, adalimumab, adapalene, adapalene+benzoyl peroxide, adapalene+clindamycin hydrochloride, adapalene+clindamycin phosphate, aderamastat, Adi, aDi-100, Adipocell, adipose tissue-derived mesenchymal stem cell-derived exosomes, adipose-derived stem cell therapy, ADMSC-CM, ADSTEM, ADX-246, aerosolized hydroxychloroquine, afamelanotide, AJ-101, AJ-303, AK-101, AK-119, AKP-08, albuterol sulfate, ALD-R491, alefacept, Allergovac depot, allogeneic adipose-derived mesenchymal stem cell therapy, allogeneic adult pluripotent stem cells, allogeneic mesenchymal stem cell therapy, allogeneic UC-MSC therapy, allogeneic umbilical cord mesenchymal stem cell therapy, AlloRx, alprazolam, AM-1476, ambroxol hydrochloride, AMG-0101, Amilo-5MER, aminolevulinic acid, aminolevulinic acid hydrochloride, aminopterin, amlitelimab, AMTX-100, AMTX-100 CF, Anapsos, ANB-032, ANB-101, anifrolumab, anti-CD19 CAR T cell therapy, anti-CD7 CAR T-cell therapy, anti-EMAP II fully humanized antibodies, anti-IL-4/IL-13 vaccine, anti-P2X7 monoclonal antibody humanized, anti-PAR2 therapeutics, antroquinonol, APD-588, APG-222, APG-777, APG-808, APG-990, APGT-001, APIRx-1603, apremilast, aprepitant, APT-101, AQ-001S, AQ-280, AR-100DP1, AR-110, arformoterol, ARG-201, ARGX-118, ARN-4079, ARO-MUC5AC, ARO-RAGE, ARO-TSLP, ARQ-234, arsenic trioxide, ARTS-011, AS-012, asengeprast, asivatrep, ASN-008, astegolimab, AT-004, AT-005, AT-0287, AT-193, ATB-1606, ATI-2138, ATL-105, ATR-006, ATR-01, ATTO-002, ATTO-1310, atuliflapon, AUR-101, auremolimab, autologous leukocyte cell therapy, avenciguat, AVI-3307, AVID-200, AVX-001, AWEPO-003, AX-158, AX-202, AZD-0284, AZD-0449, AZD-8630, azelaic acid, azelastine, azithromycin, B-244, Bacmune, bambuterol, baricitinib, barzolvolimab, BAT-6026, bazlitoran, BB-1511, BBACN, BBI-03, BBI-6000, BCG polysaccharide+nucleic acid, BCI-332, beclometasone dipropionate+formoterol fumarate, beclomethasone dipropionate, beclomethasone dipropionate+formoterol fumarate+glycopyrronium bromide, bedoradrine, begelomab, belimumab, belumosudil, bempikibart, bencycloquidium bromide, benralizumab, benzoyl peroxide, benzoyl peroxide+tretinoin, berdazimer sodium, bermekimab, bersiporocin dihydrochloride, bertilimumab, betamethasone, betamethasone dipropionate, betamethasone valerate, bexotegrast, BFP-002, BFP-102, BGB-23339, BI-1291583, BI-1323495, BI-765250, bilastine, bimekizumab, bimiralisib, BIO-11006 Inhalation Solution, BioLexa, BITT-CD4D11, BITT-CD4F10, BLR-200, BLU-808, BMS-986313, BMS-986322, BMS-986326, BMX-010, boningmycin, BOS-475, Bosakitug, bosentan, bovhyaluronidase azoximer, Box-5, BR-201, branebrutinib, BRE-AD01, brensocatib, brentuximab vedotin, brepocitinib, brilacidin, brilaroxazine hydrochloride, briquilimab, brodalumab, BSI-056T, BSI-502, BTX-1204, BTX-1308, BTX-1503, budesonide, budesonide+arformoterol, budesonide+formoterol, budesonide+formoterol fumarate, budesonide+procaterol hydrochloride, budesonide+salbutamol, budesonide+salmeterol, buloxibutid, BV-200 series, BVX-20, BZ-371, BZ-371B, C4X-6746, C-867, CABA-201, CAL-4, calcipotriol, calcipotriol+betamethasone, calcipotriol+betamethasone dipropionate, calcipotriol+cortisone, calcitriol, CALY-002, camoteskimab, CAN-10, cannabidiol, cannabidiol+dronabinol, cannabinoid CB2 receptor agonist antibody, carbon dioxide+perfluorooctyl bromide, cavosonstat, CB-06-01, CB2 receptor agonists, CB5138-3, CC-90006, CC-92252, CCI-15106, CCX-624, CD19-CAR-DNT, CEE-321, cendakimab, certolizumabpegol, CG-459, Chanllergen, CHF-6333, CHF-6366, CHF-6550, ciclesonide, ciclosporin, ciprofloxacin hydrochloride, CIT-013, CJRB-402, CKBA, clascoterone, CLBS-03, clindamycin, clindamycin phosphate+benzoyl peroxide, clindamycin phosphate+tretinoin, clobetasol propionate, clobetasol propionate+tretinoin, CM-101, CM-326, CMK-389, CMR-316, CMS-D001, ColiFin, COPD vaccine, cord blood derived stem cells, corticotropin, COYA-204, CPL-409116, crisaborole, CS-12192, CS-32582, CS-43001, CSJ-117, CSPCHA-115, CT-05, CT-303, CT-P55, CTX-101, CTXT-102, cudetaxestat sodium, CUR-N399, Cutaquig, CVXL-0074, CXF-11, CXG-86, CXG-87, cyproterone acetate+ethinyl estradiol, D-2570, D4-103-01, D4-103-02, D4-103-03, D4-103-04, daniluromer, dapansutrile, dapsone, daridorexant hydrochloride, daxdilimab, dazukibart, DB-007-4, DBI-001, DBM-1152A, DC-806, DC-853, deflazacort, delgocitinib, denifanstat, depemokimab, dersimelagon, desloratadine, desogestrel+ethinylestradiol, desonide, deucravacitinib, deuruxolitinib phosphate, dexamethasone sodium phosphate, dexpramipexole, difamilast, dimethyl fumarate, dimethyl fumarate+ethyl hydrogen fumarate calcium+ethyl hydrogen fumarate magnesium+ethyl hydrogen fumarate zinc, diroleuton, dithranol cream, divozilimab, DLQ-02, DLX-105, DLX-2323, DMT-210, DMT-310, DMX-700, DMXD-011, DNX-114, doxofylline, doxofylline (bronchiectasis), Alitair Pharmaceuticals, doxycycline hyclate, doxycycline hyclate (delayed release), Mayne, doxycycline hyclate (easy-to-swallow, acne, bacterial infection), Aqua Pharmaceuticals, DPT-0218, drospirenone+ethinylestradiol, dual alpha-V/beta-1 and alpha-5/beta-1 integrin inhibitors, dual AMCase/CHIT1 inhibitors, dual anti-CD19/anti-BAFF CAR T-cell therapy, dual JAK3/TEC inhibitor, dupilumab, dust mite vaccine, DW-20085, DYV-024, DZ-2002, EB-005, EB-06, EBI-H, eblasakimab, efzofitimod, EI-001, elapegademase, elarekibep, emedastine, empasiprubart, ENA-002, ENB-109, endonuclease modulators, ENERGI-F708, enpatoran, ensifentrine, ensifentrine+glycopyrrolate, EP-104-GI, EP-262, epeleuton, Epi-13, epinastine hydrochloride, epinephrine, EpiTight, EPM-301, EQ-101, erdosteine, erlotinib, ESK-001, etanercept, EtanerRel, ETD-001, ETH-47, etrasimod, etrinabdione, EVX-B4, EYD-001, F-200, F-528, factor D inhibitor, farudodstat, FB-102, FB-401, FB-704A, FB-825, FB-918, FCR-001, FCX-013, fevipiprant, filgotinib maleate, fipaxalparant, flunisolide, fluocinonide, fluticasone, fluticasone+formoterol, fluticasone furoate, fluticasone furoate+umeclidinium+vilanterol, fluticasone furoate+vilanterol trifenatate, fluticasone propionate, fluticasone propionate+formoterol fumarate, fluticasone propionate+salbutamol sulfate, fluticasone propionate+salmeterol, fluticasone propionate+salmeterol xinafoate, formoterol, formoterol fumarate, formoterol fumarate+fluticasone propionate, formoterol fumarate+glycopyrronium bromide, FPP-003, FPP-005, froniglutide, FRTX-02, FTC-001, FWB-1313, FZ-007, FZJ-003, GABAA receptor agonists, Gamunex, GB-001, GB-0895, GD-134, GD-iExo-001, gefurulimab, GEN-501, GL-7190, GLPG-3667, glutathione+ascorbic acid+bicarbonate, glycopyrrolate+formoterol fumarate+budesonide, glycopyrronium+formoterol fumarate+fluticasone propionate, glycopyrronium+vilanterol, glycopyrronium bromide, glycopyrronium bromide+indacaterol maleate, GMDP, GM-XANTHO, GN-037, GNKS-356, GNR-068, GPCR antagonists, GR-010, GR-1501, GR-1802, GR-2002, GR-2301, Grastek, GRC-39815, GRT-6015, GSK-1070806, GSK-2831781, GSK-3862995B, GSK-3923868, GT-20029, gumokimab, gusacitinib, guselkumab, GZ-21T, H-018, halobetasol propionate, halobetasol propionate+tazarotene, halogenated xanthene, halometasone, HB00-17, HB-0034, HB-0043, HB-1734, HBM-9001, HBM-9378, HCW-9302, HDM-3010, HECB-1800301, HEMP-001, HI-1640V, histamine human immunoglobulin, Hizentra, HJ-787, HL-231, HLA-open conformer-specific monoclonal antibody, HLK-6002, house dust mite allergen, house dust mites immunotherapy, HP-1901, Hpb glutamate dehydrogenase modulator, HPP-737, HpVac-R13, HRG-2005, HRS-9821, HS-10374, HS-401, HT-004, HuL-001, human adipose-derived mesenchymal stem cells, human umbilical cord-derived mesenchymal stem cell therapy, HY-07170702, HY-072808, HY-1770, HY-209, hypericin, hypochlorous acid, HZ-J001, IBI-3002, IBI-356, IBIO-100, IBL-101, icanbelimod, ICP-332, ICP-488, iCP-NI, ifetroban, IFNalpha kinoide, IgE inhibitors, IHL-675A, IL-17 NanoAb, IL-25 targeted therapeutic, IL-4R alpha antagonist, IL-4Ra targeted therapeutic, ILB-2107, iloprost, IMB-101, IMG-007, IMG-008, IMG-036, immune globulin intravenous, imsidolimab, IMX-120, IN-A002, inaticabtagene autoleucel, INCB-054707, indacaterol, indacaterol acetate+glycopyrronium bromide+mometasone furoate, Indamet, inebilizumab, infliximab, Integrin alpha-2/beta-1 inhibitor, Integrin alpha-5/beta-1 inhibitor, Interleukin IL-17A inhibitor, INV-007, INV-103, INV-17, IPG-1094, IPG-7236, ipratropium+fenoterol, ipratropium bromide, ipratropium bromide+salbutamol sulfate, IR-444, IRL-201104, IRX-4204, isotretinoin, itepekimab, itolizumab, ivacaftor, ivarmacitinib sulfate, ivermectin, ixekizumab, izokibep, JadiCell therapy, JAK inhibitors, JAK-989, jaktinib dihydrochloride monohydrate, jaktinib hydrochloride, JK-0001, JK-0002, JNJ-1459, JNJ-2113, JNJ-3534, JNJ-67484703, JRF-106, JRF-401, JRP-878, JS-005, JTE-051, JTE-451, JW-1601, JW-202232, JW-2202, JYB-1904, JYP-0061, JYP-0066, K-1032, KB-5XX, KBL-693, KBL-697, KI-696, KINE-201, KITCL-27, KN-002, KP-470, KT-294, KT-474, KT-621, KX-826, KYV-101, L-608, LABA+LAMA therapy, Langopept, larsucosterol, LAS-200019, LBG-1600M, LCK inhibitor, lebrikizumab, lepzacitinib, levalbuterol, levalbuterol hydrochloride, levonorgestrel+ethinylestradiol, LG-283, LGM-1506, LGM-2605, LH-8, LIT-00505, lithium succinate, LMY-920, LNK-01001, LNK-01004, LNP-1955, LNR-653.1, londamocitinib, long acting beta agonist/long acting muscarinic agonist, long-acting aerosolized peptide-based therapy, lonodelestat acetate, lp-003, LP-0200, LQ-036, LQ-041, LQ-043, LR-19019, LR-20016, LT-002-158, lucinactant, lunsekimig, LUT-014, LW-104, LY-3509754, LY-3872386, LY-3972406, LYS-006, lysophospholipase inhibitor, LZM-012, M-119102, M3 muscarinic receptor antagonists, M-605110, M610101, manfidokimab, masitinib, MAX-40070, maxacalcitol, maxacalcitol+betamethasone, MCM-001, MDI-1228, MDNA-413, MDPK-67b, ME-3183, Melgain, mepolizumab, mesalazine, Mesenchymal stem/stromal cell therapy, mesenchymoangioblast-derived mesenchymal stem cell therapy, metenkefalin acetate+tridecactide acetate, methotrexate, methyl aminolevulinate hydrochloride, methylprednisolone suleptanate, MG-01, MG-K10, MG-S-2525, MGY-1838, MGZG-122, MH-004, MH-080, minocycline, minocycline+adapalene, minocycline hydrochloride, MIT-001, mitiperstat, Mitizax, MM-09, mometasone, mometasone+formoterol, mometasone furoate, mometasone furoate+indacaterol acetate, monlunabant, montelukast, montelukast sodium, montelukast sodium+levocetirizine dihydrochloride, mosedipimod, mouse monoclonal antibody against human interleukin-8, MP-1032, MSB-01, MSB-03, MSB-3163, MSM-605, MT-5562, MTC-896, mucosa-associated lymphoid tissue lymphoma translocation protein 1 inhibitors, mufemilast, MufroSyn, mugwort pollen allergen vaccine, muscarinic M3 receptor antagonist, MYJ-1633, nacystelyn, nadifloxacin, nadolol, nalfurafine, NBL-012, NCP-111, NCP-112, ND-003, NDX-3315, NDX-3324, nedocromil, nemolizumab, netakimab, nibrozetone, niclosamide, NIK inhibitors, nitric oxide, nitroglycerin, NLP-91, NM26-2198, nomacopan, norethindrone acetate+ethinylestradiol, noscapine/noscapine analogs, NP-339, nrf2 activator, NS-402, NTR-441, NVS-451, NX-73, OATD-01, OB-756, obefazimod, OC-701, OCR-4715, Octagam 10%, olodaterol, olodaterol hydrochloride+tiotropium bromide monohydrate 1, olopatadine, OLX-103, OM-001, omalizumab, omiganan pentahydrochloride, omilancor, OMN-71, ONO-4685, OP-2101, opinercept, OpSCF, ordesekimab, ORI-001, orismilast, ORKA-001, ORKA-002, orticumab, ozagrel hydrochloride, ozenoxacin, PA-9159, paridiprubart, PBF-680, PBI-100, PC-114, PDC-APB, PDE4 inhibitor, pegtarazimod, pemirolast, peresolimab, PF-07264660, PF-07275315, PG-011, PG-102, Viromed, phimelanotide, PHP-1212, PI3K-delta inhibitor, picankibart, piclidenoson, pimecrolimus, PIPE-791, pirfenidone, pitavastatin, PKC theta inhibitors, PLM-301, PNV-5032, POLB-002, ponesimod, potassium dobesilate, PR-023, pranlukast, pranlukast hydrate, PRCL-02, PrEP-001, prostaglandin D2 synthase inhibitors, Prozumab, PRP-PBMC autologous cellular therapy, PS-35, psoriasis therapeutics, PT-101, P-TET, PUL-042, PUR-0110, PUR-1800, PX-128, PX-130, PZ-07/2024, Q-1804, Q-301, QBKPN, QLM-3003, QN-02, QP-CO1, QRX-008, quisovalimab, QX-002-N, QX-004-N, QX-005-N, QX-007-N, QX-008-N, QX-009-N, QX-010-N, QY-101, QY-201, QY-211, R-187, R-552, rademikibart, rare phytocannabinoids, ravulizumab, RAY-121, RB-1000, RBM-009, RBN-012759, RBO-0987, RC-1416, RCD-405, recombinant midismase, reformulated calcipotriol+betamethasone, REGEND-001, REGN-1908-1909, remetinostat, remibrutinib, renzapride, repirinast, repurposed aldesleukin, Repurposed azeliragon, repurposed lenabasum, reslizumab, RESP-1000 series, RESP-2000 series, RESP-X, retinoic acid, revefenacin, REX-7117, rezpegaldesleukin, RG-6151, RG-6244, RG-6314, RG-6315, RG-6341, RG-6421, RGRN-305, rilzabrutinib, riociguat, risankizumab, ritlecitinib, rituximab, RLS-1496, RLV-102, rocatinlimab, roflumilast, ropsacitinib, ROR gamma T inverse agonists, ROR-gamma inverse agonists, rose bengal sodium, RP-3128, RSBT-001, RSS-0393, R-TPR-022, rupatadine+montelukast, RUTI, ruxolitinib, RYSW-01, S1P1 agonist, salbutamol, salmeterol, salmeterol xinafoate+fluticasone propionate, SAMiRNA program, SAR-441566, SAR-443726, sarecycline, SB-010, SB-011, SCD-044, SCD-153, SCT-640A, SCT-650-C, SDC-1801, secukinumab, SEGRA, seletalisib, SEL-K2, selnoflast, seratrodast, SFA-002, SFA-004, SG-100, SGT-510, SH2 domain inhibitor program targeting STAT6, SHR-1703, SHR-1819, SHR-1905, SHR-4597, si-544, SIG-1322, SIG-1451, SIG-1456, SIM-0278, SIM-335, sitaxentan, SKI-O-703, SKL-XYZ, SLS-008, SM-17, small mobile stem cell therapy, SMET-D1, SNC-103, SNG-001, SNG-100, SNK-01, sodium chromoglycate, sodium pyruvate, sonelokimab, soquelitinib, sovleplenib, spesolimab, SPL84-23, SSGJ-621, SSS-07, ST-1830, stapokibart, STAR-0310, STAT3 inhibitor, STMC-103H, STS-01, STSA-1201, SUDO-286, SUL-238, SuperMApo, suplatast tosilate, SYHX-1901, SYX-5219, T-517, tacalcitol, tacrolimus, TAF-001, tagraxofusp, TAGX-0003, TAKC-02, tanimilast, TAP-1502, TAP-1503, tapinarof, Tavo-101, tazarotene, tazarotene+betamethasone dipropionate, tazarotene+clindamycin, TD-8236, TDM-180935, TDM-Atop01, TDM-Psor01, TDM-Scar01, telazorlimab, Tempol, temtokibart, teprotumumab, TER-101, terbutalin, terguride, tesnatilimab, TEV-48574, TEV-53275, tezepelumab, TFF-HMW-HA, Thalassophryne nattereri peptide, THB-001, theophylline, THOR-809, TI-520, TI-620, tibulizumab, tildrakizumab, timolumab, tiotropium, tiotropium bromide, tipelukast, tirbanibulin, TLL-018, TLY-012, TO-210, tofacitinib, tofacitinib+fingolimod, tofacitinib citrate, tonabacase, TOP-N44, TOP-N53, torudokimab, tosufloxacin, tozorakimab, TP-317, TQC-2731, TQC-2938, TQC-3564, TQC-3721, TQC-3927, TQH-2722, TQH-2929, TQH-3906, TQH-3910, trabikibart, tralokinumab, tranilast, transcription factor pathway inhibitor, tregalizumab, treprostinil, treprostinil diolamine, tretinoin, tretinoin+benzoyl peroxide, trifarotene, TRIV-509, TRN-157, TRPA1 antagonists, TS-0001, TT-01, TT-01688, tulinercept, tulobuterol, TVB-3567, UA-021, UB-221, UCB-1381, UCB-9741, ucenprubart, UHE-101, UHE-105, UI-009, UI-010, UI-031, UI-033, UI-034, ulobetasol, umbilical cord blood-derived stem cell therapy, UMC119-06, umeclidinium bromide, umeclidinium bromide+vilanterol trifenatate, upadacitinib, USP-4 inhibitors, ustekinumab, UTAA-09, VALERGEN-DS, vamorolone, vapendavir, vardenafil, VB-1953, VC-005, VDAA, VDAD, VDJ-006, VEGFR targeted DK4/10, venanprubart, VENT-03, verekitug, vilanterol+fluticasone furoate+glycopyrronium bromide, vipoglanstat hydrogensulfate, VISTA agonist, vixarelimab, VLRX-001, VM-AD, VNLG-152, vonifimod, voriconazole, votucalis, VRN-04, VS-105, VSG-158, VSTM-1 agonist, VT-014, VTH-212, vunakizumab, VYN-201, VYN-202, W16P-0576, WD-890, WM-1R3, WNT inhibitor, WNT pathway agonist antibodies, wnt pathway stimulator, WXFL-10203614, WXSH-0150, XCUR-17, XKH-001, XmAb-564, XT-0528, XZ.700, YH-25487, YH-35324, YKRH-00020, YR-001, Yso-3, zabedosertib, zafirlukast, zasocitinib, ZB-168, Zemaira, ZeP-3, zetomipzomib, zevaquenabant, ZHB-107-108, zibotentan, zileuton, zirconium zr 89 crefmirlimab berdoxam, ZL-1102, ZPL-521, and/or bi-specific antibodies targeting one or more targets referenced herein.

In some embodiments a compound of the disclosure provided herein, or pharmaceutically acceptable salt thereof, is administered with one or more therapeutic agents selected from a PPARδ inhibitor, IRAK4 inhibitor, TPL2 inhibitor, α4β7 inhibitor, BTLA agonist, PD1 agonist, an ACC inhibitor, a GLP1 agonist, or an FXR agonist.

In some embodiments a compound of the disclosure provided herein, or pharmaceutically acceptable salt thereof, is administered with one or more therapeutic agents selected from seladelpar, edecesertib, tilpisertib fosmecarbil, GS-1427, GS-0272, GS-0151, GS-6791, or cilofexor.

### IX. Compound Preparation

In some embodiments, the present disclosure provides processes and intermediates useful for preparing the compounds disclosed herein or pharmaceutically acceptable salts thereof.

Compounds disclosed herein can be purified by any of the means known in the art, including chromatographic means, including but not limited to high-performance liquid chromatography (HPLC), preparative thin layer chromatography, flash column chromatography, ion exchange chromatography, and supercritical fluid chromatography (SFC). Any suitable stationary phase can be used, including but not limited to, normal and reversed phases as well as ionic resins. In some embodiments, the disclosed compounds are purified via silica gel and/or alumina chromatography.

During any of the processes for preparation of the compounds provided herein, it can be necessary and/or desirable to protect sensitive or reactive groups on any of the molecules concerned. This can be achieved by means of conventional protecting groups as described in standard works, such as T. W. Greene and P. G. M. Wuts, PROTECTIVE GROUPS IN ORGANIC SYNTHESIS, 4th ed., Wiley, New York 2006. The protecting groups can be removed at a convenient subsequent stage using methods known in the art.

Exemplary chemical entities useful in methods of the embodiments will now be described by reference to illustrative synthetic schemes for their general preparation herein and the specific examples that follow. Skilled artisans will recognize that, to obtain the various compounds herein, starting materials can be suitably selected so that the ultimately desired substituents will be carried through the reaction scheme with or without protection as appropriate to yield the desired product. Alternatively, it can be necessary or desirable to employ, in the place of the ultimately desired substituent, a suitable group that can be carried through the reaction scheme and replaced as appropriate with the desired substituent. Furthermore, one of skill in the art will recognize that the transformations shown in the schemes below can be performed in any order that is compatible with the functionality of the particular pendant groups.

The methods of the present disclosure generally provide a specific enantiomer or diastereomer as the desired product, although the stereochemistry of the enantiomer or diastereomer was not determined in all cases. When the stereochemistry of the specific stereocenter in the enantiomer or diastereomer is not determined, the compound is drawn without showing any stereochemistry at that specific stereocenter even though the compound can be substantially enantiomerically or diastereomerically pure.

Compounds disclosed herein can be prepared from commercially available reagents using the synthetic methods and reaction schemes described herein, or using other reagents and conventional methods known to persons of ordinary skill in the art. For instance, representative syntheses of compounds of the present disclosure are described in the schemes below, and the particular examples that follow.

### X. General Schemes

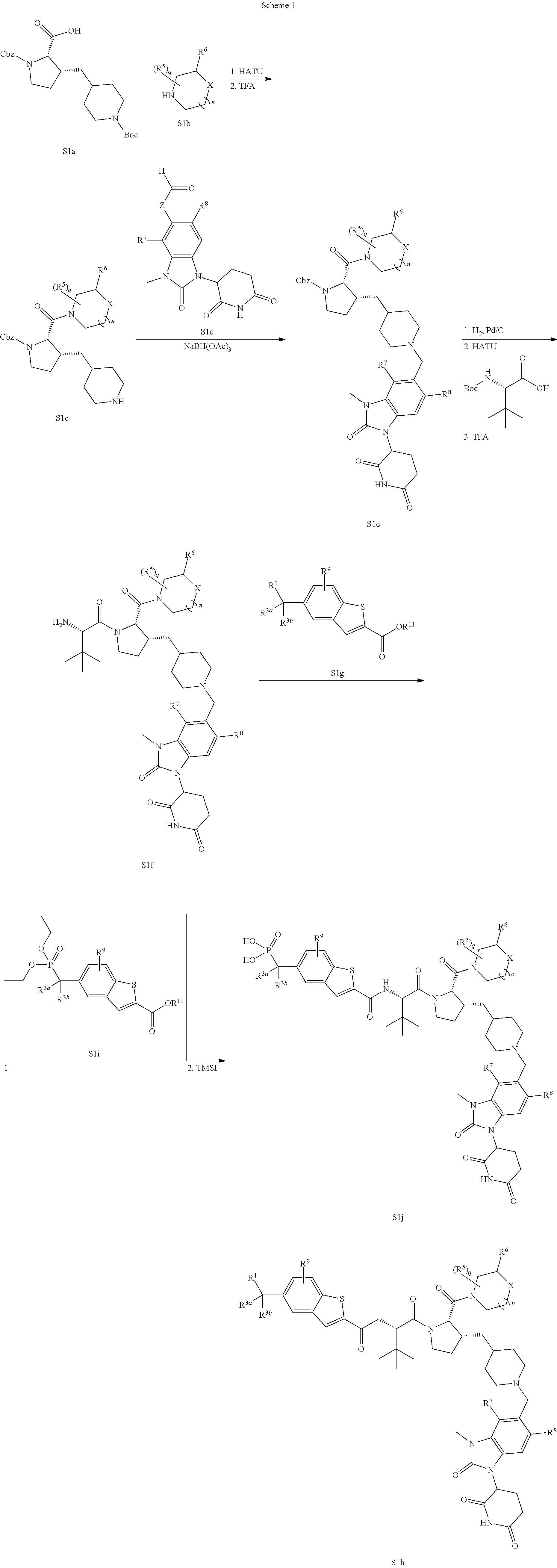

Scheme 1 shows a general synthesis of compounds beginning with the coupling of an acid (e.g. S1a) to an amine (e.g. S1b) in the presence of an amide coupling reagent (e.g. HATU) with subsequent protecting group (e.g. Boc) deprotection under acidic conditions (e.g. TEA) can afford amine (e.g. S1c). A reductive amination with aldehyde (e.g. S1d) wherein Z is C₁-C₃ alkyl, C₁-C₃ haloalkyl, C₃-C₆ cycloalkyl, or a 4- to 7-membered heterocycloalkyl having 1 to 3 heteroatoms each independently N, O, or S, wherein the bond connecting the aldehyde and heterocyclyl group is a carbon-carbon bond, under reducing conditions (e.g. NaBH(OAc)₃, NaBH₃CN) can afford an amine (e.g S1e). Removal of the protecting group (e.g. Cbz) under reducing conditions (e.g. hydrogen, Pd/C) and subsequent coupling to Boc-Tle-OH in the presence of an amide coupling reagent (e.g. HATU) with subsequent protecting group (e.g. Boc) removal under acidic conditions (e.g. TFA) can afford an amine (e.g. S1f). The amine (e.g. S1f) is coupled with an ester or acid of the type S1g wherein R¹¹ is hydrogen or an activating group (e.g. perfluorophenyl or 4-nitro-phenyl) affording final compounds (e.g. examples 5.4, 5.5) of the type S1h. Alternatively, the amine S1f can be coupled with an ester or acid of the type S1i wherein R¹¹ is hydrogen or an activating group (e.g. perfluorophenyl or 4-nitrophenyl) with subsequent deprotection in the presence of TMSI can afford final compounds (e.g. examples 2.1, 2.2, 3.3) of the type S1j.

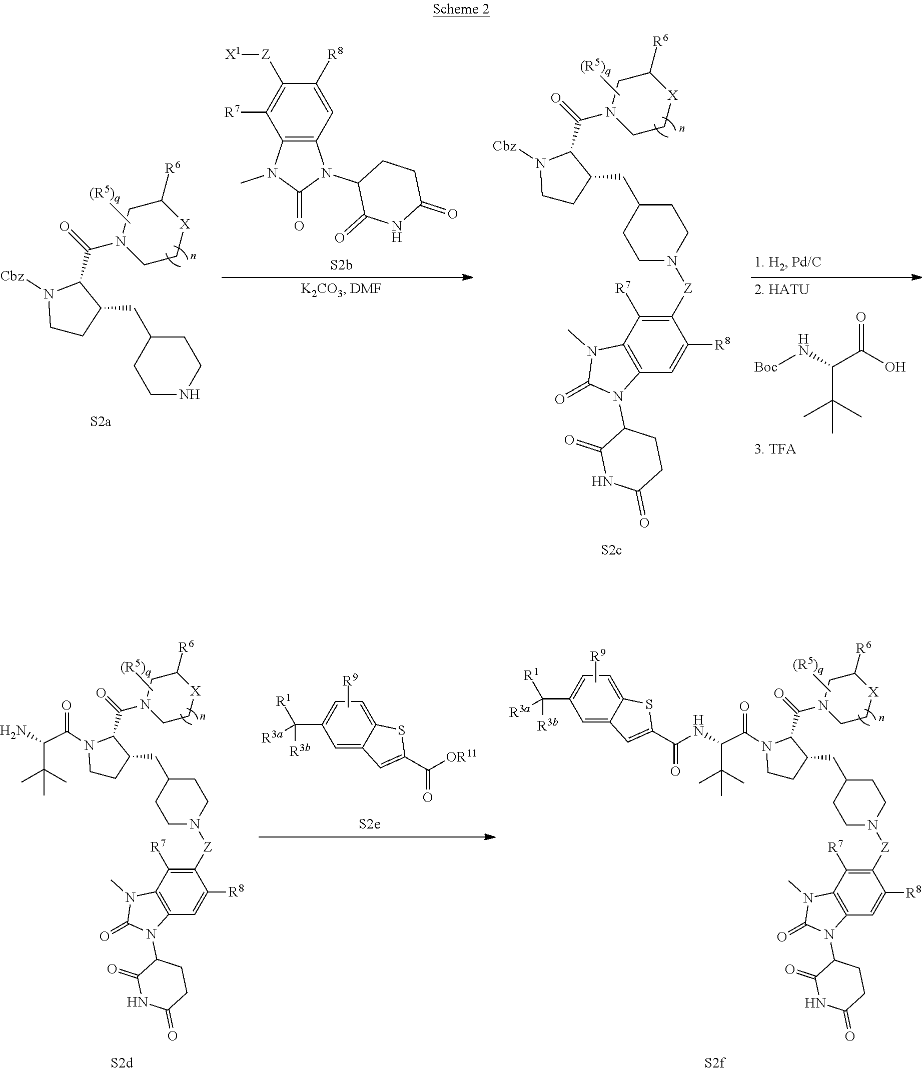

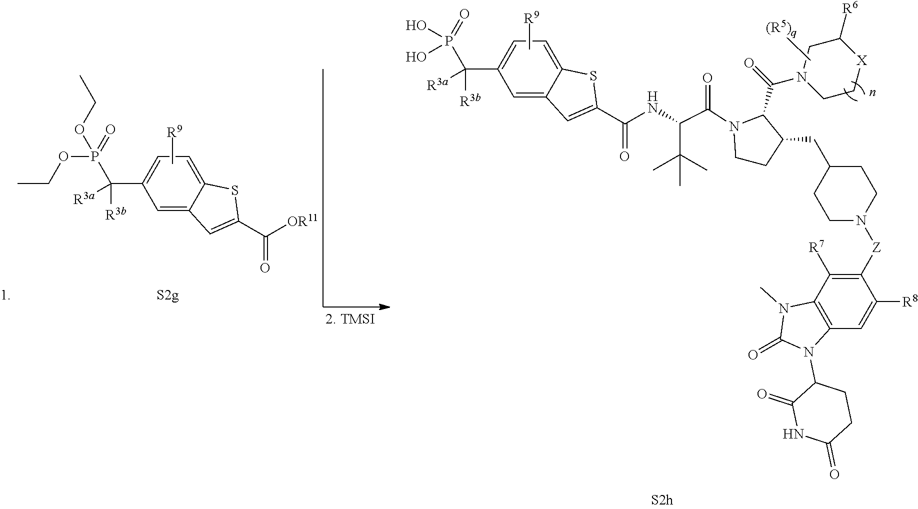

Scheme 2 shows a general synthesis of compounds beginning with the nucleophilic displacement of an alkyl halide (e.g. S2b) by an amine (e.g. S2a) under basic conditions (e.g. K₂CO₃, DMF) can afford a compound of the type S2c, wherein X¹ is a leaving group (e.g. chloride, bromide, iodide, mesylate, tosylate, triflate) and Z is C₁-C₃ alkyl, C₁-C₃ haloalkyl, C₃-C₆ cycloalkyl, or a 4- to 7-membered heterocycloalkyl having 1 to 3 heteroatoms each independently N, O, or S, wherein the bond connecting the aldehyde and heterocyclyl group is a carbon-carbon bond. Removal of the protecting group (e.g. Cbz) under reducing conditions (e.g. hydrogen, Pd/C), subsequent coupling to Boc-Tle-OH in the presence of an amide coupling reagent (e.g. HATU), and subsequent deprotection (e.g. Boc removal) under acidic conditions (e.g. TEA) can afford an amine (e.g. S2d). The amine (e.g. S2d) can be coupled with an ester or acid of the type S2e wherein R¹¹ is hydrogen or an activating group (e.g. perfluorophenyl or 4-nitrophenyl) which can afford final compounds (e.g. examples 5.4, 5.5) of the type S2f. Alternatively, the amine S2d can be coupled with an ester or acid of the type S2g wherein R¹¹ is hydrogen or an activating group (e.g. perfluorophenyl or 4-nitrophenyl) with subsequent deprotection in the presence of TMSI can afford final compounds (e.g. examples 2.1, 2.2, 3.3) of the type S2h.

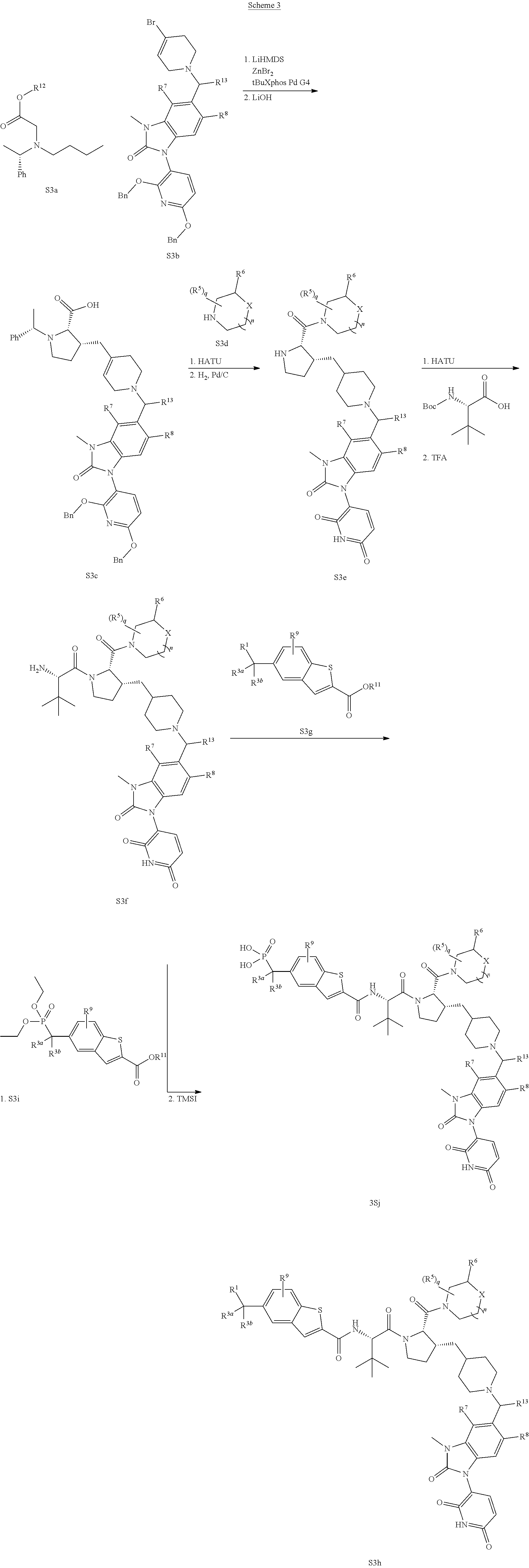

Scheme 3 shows a general synthesis of compounds beginning with the base and metal-mediated (e.g. LiHMDS, ZnBr₂, tBuXphos Pd G4) cyclization of S3a, wherein R¹² is an alkyl group (e.g. methyl, ethyl, tert-butyl) that is optionally substituted with phenyl (benzyl) and in situ coupling of a vinyl halide (e.g. S3b), wherein R¹³ is independently hydrogen, C₁-C₃ alkyl, or C₁-C₃ haloalkyl, followed by ester hydrolysis (e.g. under basic conditions with LiOH) can afford an acid (e.g. S3c). Coupling of an acid of the type S3c to an amine (e.g. S3d) in the presence of an amide coupling reagent (e.g. HATU) with subsequent global reduction and protecting group (e.g. Cbz) removal under reducing conditions (e.g. hydrogen, Pd/C) can afford an amine (e.g. S3e). Coupling of an amine of the type S3e to Boc-Tle-OH in the presence of an amide coupling reagent (e.g. HATU) with subsequent protecting group (e.g. Boc) removal under acidic conditions (e.g. TFA) can afford an amine (e.g. S3f). The amine (e.g. S3f) can be coupled with an ester or acid of the type S3g wherein R¹¹ is hydrogen or an activating group (e.g. perfluorophenyl or 4-nitro-phenyl) can afford final compounds (e.g. examples 5.4, 5.5) of the type S3h. Alternatively, the amine S3f can be coupled with an ester or acid of the type S3i wherein R¹¹ is hydrogen or an activating group (e.g. perfluorophenyl or 4-nitrophenyl) with subsequent deprotection in the presence of TMSI can afford final compounds (e.g. examples 2.1, 2.2, 3.3) of the type S3j.

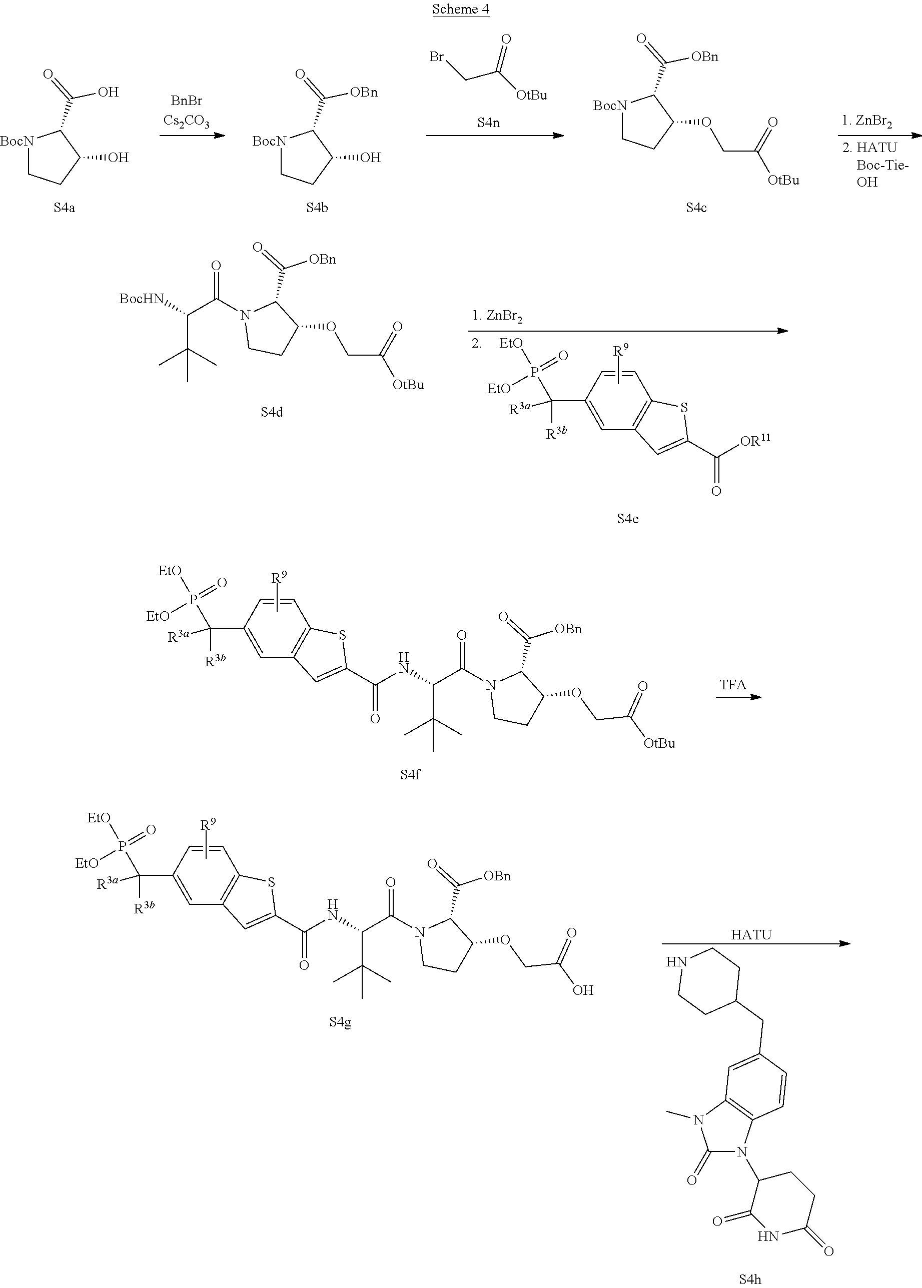

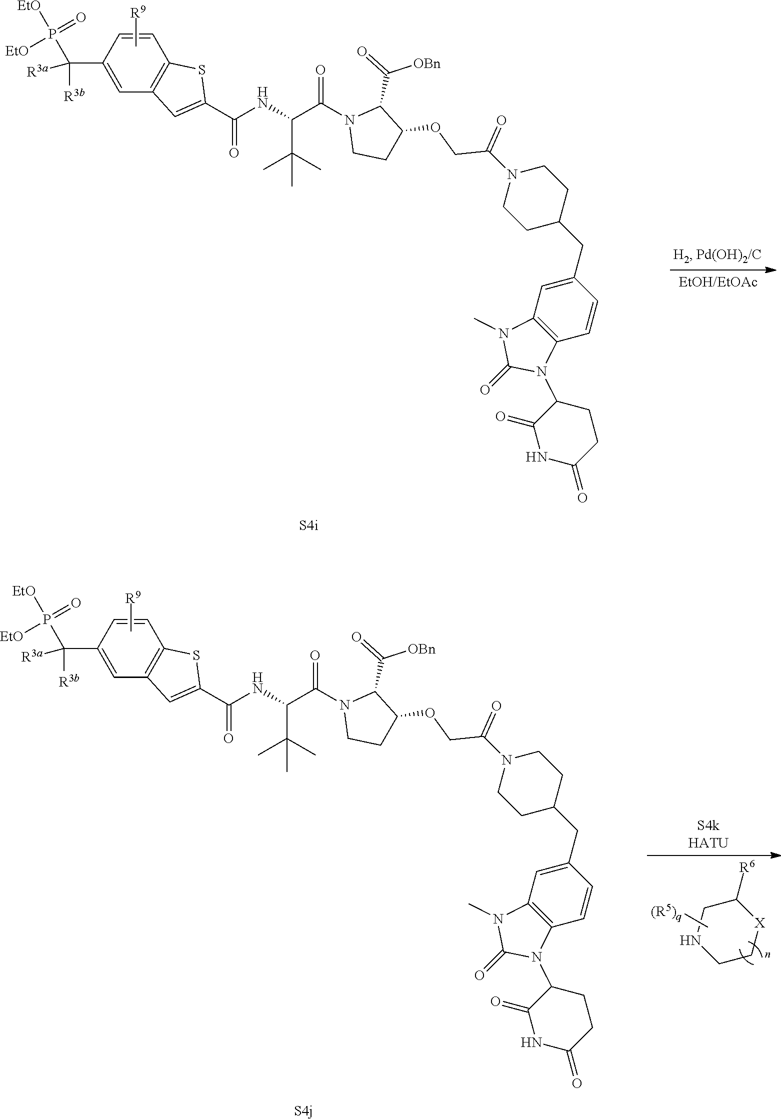

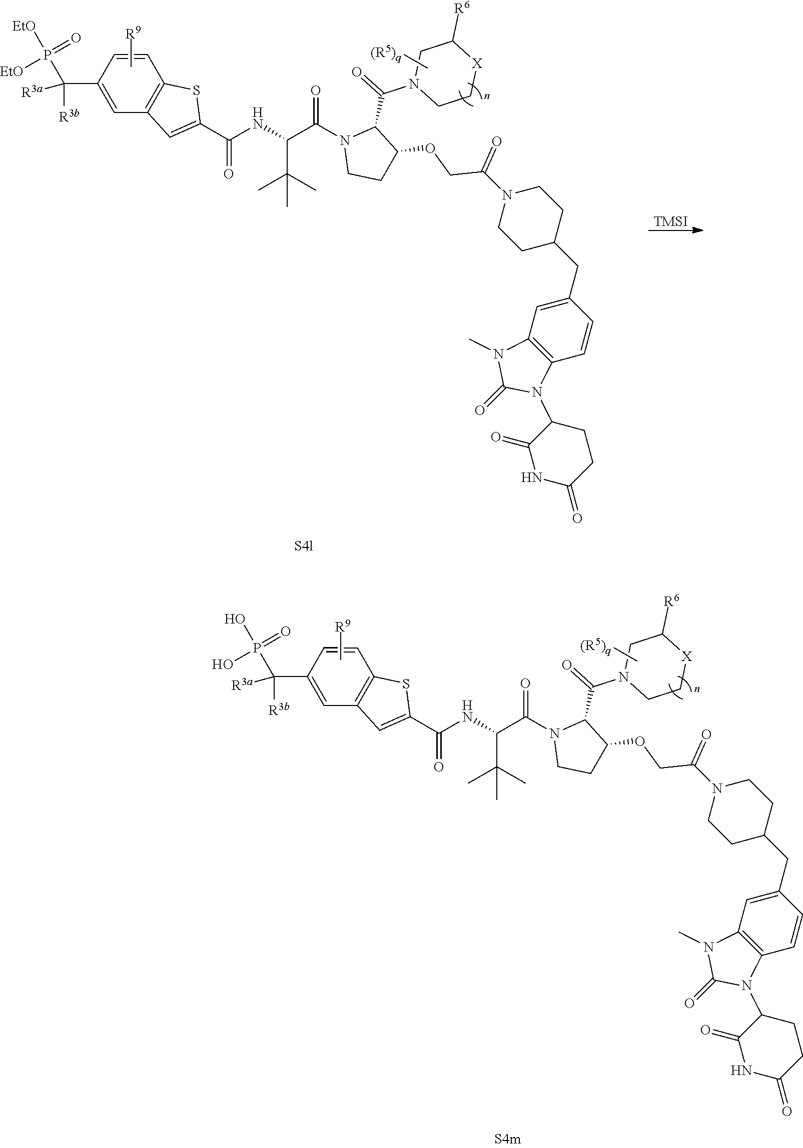

Scheme 4 shows a general synthesis of compounds beginning with the protection (e.g. benzylation) of a hydroxy acid (e.g. S4a) with an alkyl halide (e.g. benzyl bromide) and base (e.g. Cs₂CO₃) to afford an alcohol (e.g. S4b). Alcohol S4b is then alkylated with an alkyl halide (e.g. S4n) in the presence of base (e.g. Cs₂CO₃) can afford a product of the type S4c. Compound S4c is then deprotected (e.g. Boc group removal) in the presence of a Lewis acid (e.g. ZnBr₂) affording an amine which is then coupled to Boc-Tle-OH in the presence of an amide coupling reagent (e.g. HATU) can afford a compound of the type S4d. Compound S4d is then deprotected (e.g. Boc group removal) in the presence of a Lewis acid (e.g. ZnBr₂) affording an amine which is then coupled with an ester or acid of the type S4e wherein R¹¹ is hydrogen or an activating group (e.g. perfluorophenyl or 4-nitrophenyl) can afford a compound of the type S4f. Deprotection (e.g. t-Bu ester removal) in the presence of acid (e.g. TFA) affords a carboxylic acid (e.g. S4g) which can be then coupled to an amine (e.g. S4h) in the presence of an amide coupling reagent (e.g. HATU) to afford a compound of the type S4i. Removal of the protecting group (e.g. Bn) under reducing conditions (e.g. hydrogen, Pd/C) can afford an acid (e.g. S4j) which can then be coupled to an amine (e.g. S4k) in the presence of an amide coupling reagent (e.g. HATU) to afford compound of the type S4l. Deprotection of S4l in the presence of TMSI can afford final compounds (e.g. examples 1.17, 1.18) of the type S4m.

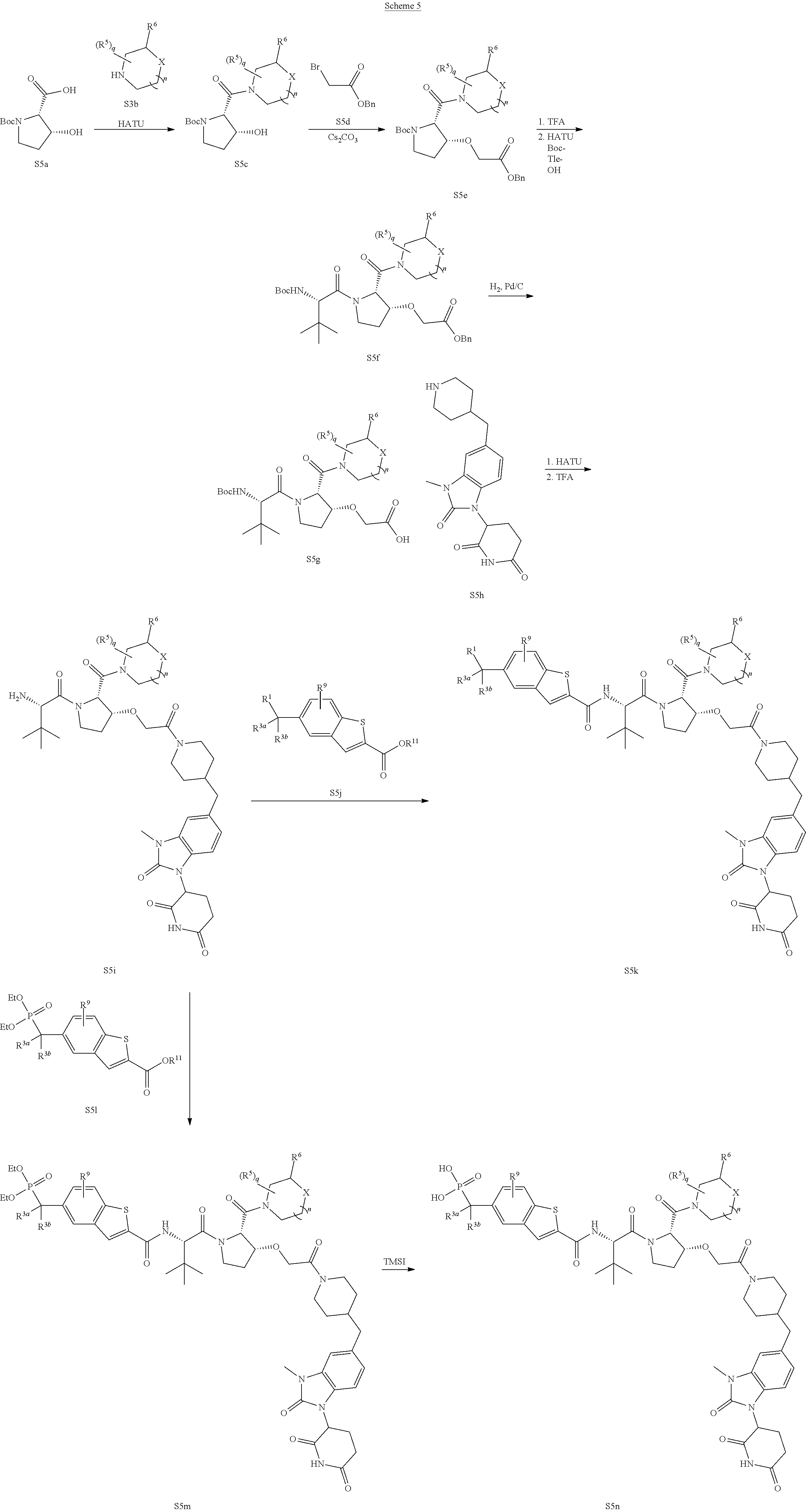

Scheme 5 shows a general synthesis of compounds beginning with the coupling of an acid (e.g. S5a) to an amine (e.g. S5b) in the presence of an amide coupling reagent (e.g. HATU) which can afford a compound of the type S5c. The alcohol (e.g. S5c) can then be alkylated with an alkyl halide (e.g. S5d) in the presence of base (e.g. Cs₂CO₃) to afford a compound of the type S5e. Compound S5e can then be deprotected (e.g. Boc removal) in the presence of acid (e.g. TFA) to afford an amine which can then be coupled to Boc-Tle-OH in the presence of an amide coupling reagent (e.g. HATU) to afford a compound of the type S5f. Removal of the protecting group (e.g. Cbz) under reducing conditions (e.g. hydrogen, Pd/C) can afford an acid (e.g. S5g) which can then be coupled to an amine (e.g. S5h) in the presence of an amide coupling reagent (e.g. HATU) to afford an amine (e.g. S5i). The amine (e.g. S5i) can be coupled with an ester or acid of the type S5j wherein R¹¹ is hydrogen or an activating group (e.g. perfluorophenyl or 4-nitrophenyl) to afford final compounds (e.g. examples 5.1, 5.2, 5.3, 5.6) of the type S5k. Alternatively, the amine S5i can be coupled with an ester or acid of the type S5l wherein R¹¹ is hydrogen or an activating group (e.g. perfluorophenyl or 4-nitrophenyl) with subsequent deprotection in the presence of TMSI to afford final compounds (e.g. examples 3.1, 3.2) of the type S5n.

### EXAMPLES

### I. Abbreviations

Certain abbreviations and acronyms are used in describing the experimental details. Although most of these would be understood by one skilled in the art, Table 1 contains a list of many of these abbreviations and acronyms.

TABLE 1
List of Abbreviations and Acronyms

| Abbreviation | Meaning |
|---|---|
| ° C. | degree(s) Celsius |
| μg or ug | microgram(s) |
| μL or uL | microliter(s) |
| μm or um | micron(s) |
| μmol or umol | micromole(s) |
| ACN | Acetonitrile |
| Aq | Aqueous |
| BAIB | iodobenzene diacetate |
| Bn | Benzyl |
| Boc | tert-butoxycarbonyl |
| br s | broad singlet |
| BSTFA | N,O-Bis(trimethylsilyl)trifluoroacetamide |
| tBu | tert-butyl |
| n-BuLi | n-butyllithium |
| Cbz | Benzyloxycarbonyl |
| D | Doublet |
| DBU | 1,8-diazabicyclo[5.4.0]undec-7-ene |
| DCM | Dichloromethane |
| dd | doublet of doublets |
| ddd | doublet of doublet of doublets |
| ddt | doublet of doublet of triplets |
| DIEA, DIPEA | N,N-diisopropylethylamine |
| DMA | Dimethylacetamide |
| DMAP | 4-dimethylamiopyridine |
| DME | 1,2-dimethoxyethane |
| DMF | Dimethylformamide |
| DMP | Dess-Martin periodinane |
| DMSO | dimethyl sulfoxide |
| dt | doublet of triplets |
| Et | Ethyl |
| EtOAc | ethyl acetate |
| Fmoc | fluorenylmethoxycarbonyl |
| G | gram(s) |
| H | hour(s) |
| HATU | hexafluorophosphate azabenzotriazole |
| | tetramethyl uronium |
| HPLC | high-performance liquid chromatography |
| Hz | Hertz |
| IPA or iPrOH | isopropyl alcohol |
| J | coupling constant |
| KHMDS | potassium bis(trimethylsilyl)amide |
| KOAc | potassium acetate |
| LAH | lithium aluminum hydride |
| LCMS | liquid chromatography mass spectrometry |
| LDA | lithium diisopropylamide |
| LED | Light emitting diode |
| tLeu | tert-Leucine |
| LiHMDS | lithium bis(trimethylsilyl)amide |
| m | multiplet |
| M | Molarity |
| Me | Methyl |
| MeCN | acetonitrile |
| MeOH | methanol |
| mg | milligram(s) |
| MHz | megahertz |
| min | minute(s) |
| mL | milliliter(s) |
| mm | millimeter(s) |
| mmol | millimole(s) |
| MOMO or | methoxymethyl |
| OMOM | |
| NaHMDS | sodium bis(trimethylsilyl)amide |
| NaOMe | sodium methoxide |
| NBS | N-bromosuccinimide |
| NFSI | N-Fluorobenzenesulfonimide |
| NMI | 1-methylimidazole |
| NMR | nuclear magnetic resonance |
| OSEM | 2-(trimethylsilyl)ethoxymethoxy |
| OTf | trifluoromethanesulfonate |
| PFP | Pentafluorophenyl |
| Ph | Phenyl |
| Piv | Pivaloyl |
| Pr | Propyl |
| iPr | iso-propyl |
| qd | quartet of doublets |
| rt | room temperature |
| s | Singlet |
| SFC | supercritical fluid chromatography |
| t | Triplet |
| TBAF | tetrabutylammonium fluoride |
| TBDPS | Tert-butyldiphenylsilane |
| TBS | tert-butyldimethylsilyl |
| TBSCI | tert-Butyldimethylsilyl chloride |
| TCFH | chloro-N,N,N′,N′-tetramethylformamidinium |
| | hexafluorophosphate |
| td | triplet of doublets |
| TEA | triethylamine |
| TEMPO | 2,2,6,6-Tetramethyl-1-piperidinyloxy |
| TES | triethylsilane |
| TFA | trifluoroacetic acid |
| TF₂O or Tf₂O | trifluoromethanesulfonic anhydride |
| THF | tetrahydrofuran |
| TIPS | triisopropylsilyl |
| TMS | trimethylsilyl |
| TMSI | iodotrimethylsilane |
| tt | triplet of triplets |
| TTMSS | Tris(trimethylsilyl)silane |
| v/v | volume/volume |
| wt | Weight |
| δ | parts per million referenced to residual |
| | non-deuterated solvent peak |
| * | Single stereoisomer (R or S), but unknown |
| | which stereoisomer |

### II. Procedures

Unless otherwise stated, proton nuclear magnetic resonance spectra are reported in pants per million (ppm) on the δ scale and are referenced from the residual protium in the NMR solvent (CDCl₃: δ 7.26, CD₃OD: δ 3.31, DMSO-d₆: δ 2.50, CD₃CN: δ 1.94, D₂O: δ 4.79). Data is reported as follows: chemical shift [multiplicity (s=singlet, d=doublet, t=triplet, q=quartet, p=pentet, sep=septet, m=multiplet, br=broad, app=apparent), coupling constants (J) in Hertz, integration. Data is reported as follows: chemical shift [multiplicity (d=doublet), coupling constants (J) in Hertz. Peak integration was conducted over regions deemed to have sufficient baseline separation and respective proton counts are provided for the specified region.

### III. Intermediates

### Preparation of 3-(5-((3,8-diazabicyclo[3.2.1]octan-3-yl)methyl)-3-methyl-2-oxo-2,3-dihydro-1H-benzo[d]imidazol-1-yl)piperidine-2,6-dione (A1.2)

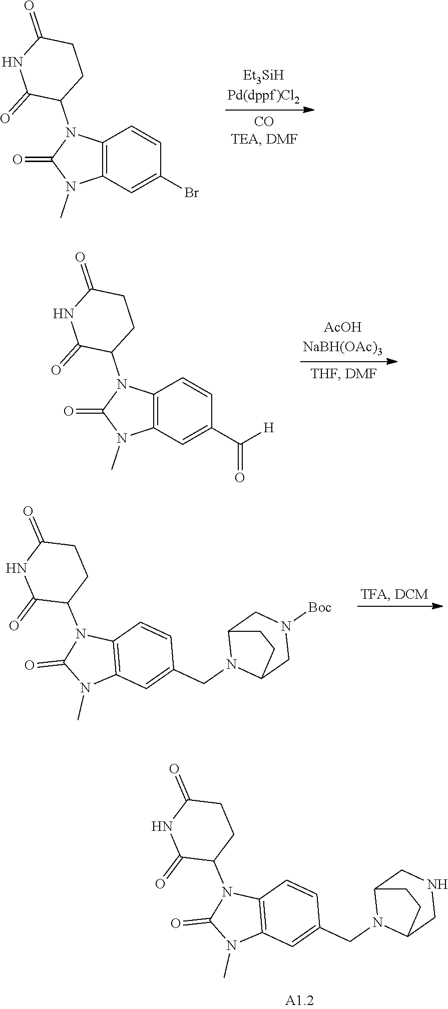

1-(2,6-dioxopiperidin-3-yl)-3-methyl-2-oxo-2,3-dihydro-1H-benzo[d]imidazole-5-carbaldehyde (A1.1) To a solution of 3-(5-bromo-3-methyl-2-oxo-2,3-dihydro-1H-benzo[d]imidazol-1-yl)piperidine-2,6-dione (32 g, 94.6 mmol, 1 eq), TEA (40 mL, 284 mmol, 3 eq), Pd(dppf)Cl₂ (7.2 g, 9.6 mmol, 0.1 eq) and Et₃SiH (32.8 g, 284 mmol, 3 eq) was added in DMF (800 mL) and after stirring at 80° C. for 18 h under CO (15 psi). The reaction mixture was diluted with EtOAc. The organic layer was washed with brine, dried over Na₂SO₄, filtered, concentrated to the crude product which was further purified by beating with EtOAc/MeCN to give A1.1. ¹H NMR (400 MHz, DMSO-d6) δ: 11.16 (s, 1H), 9.94 (s, 1H), 7.70-7.69 (m, 2H), 7.36 (t, J=4 Hz, 1H), 5.50-5.45 (m, 1H), 3.42 (s, 3H) 2.95-2.86 (m, 1H), 2.79-2.62 (m, 2H), 2.09-1.98 (m, 1H). ES/MS: m/z=288.1 [M+1]⁺.

tert-butyl 3-((1-(2,6-dioxopiperidin-3-yl)-3-methyl-2-oxo-2,3-dihydro-1H-benzo[d]imidazol-5-yl)methyl)-3,8-diazabicyclo[3.2.1]octane-8-carboxylate. A stirred solution of 1-(2,6-dioxo-3-piperidyl)-3-methyl-2-oxo-benzimidazole-5-carbaldehyde (150 mg, 0.522 mmol), tert-butyl 3,8-diazabicyclo[3.2.1]octane-3-carboxylate (222 mg, 1.04 mmol), acetic acid (0.09 mL, 1.57 mmol) in THF (1 mL) and DMF (1 mL) was treated with sodium triacetoxyborohydride (332 mg, 1.57 mmol) at room temperature. The reaction mixture was stirred for 16 h then was basified with saturated aqueous sodium bicarbonate. The mixture was extracted with ethyl acetate (3×5 mL), and the combined organic layers were washed with water (3×10 mL), dried over MgSO₄, and concentrated. The crude residue was purified by silica gel column chromatography (0-20% MeOH/DCM) to afford tert-butyl 8-[[1-(2,6-dioxo-3-piperidyl)-3-methyl-2-oxo-benzimidazol-5-yl]methyl]-3,8-diazabicyclo[3.2.1]octane-3-carboxylate. MS (ESI): m/z=484.2 [M+H]⁺.

3-(5-((3,8-diazabicyclo[3.2.1]octan-3-yl)methyl)-3-methyl-2-oxo-2,3-dihydro-1H-benzo[d]imidazol-1-yl)piperidine-2,6-dione (A1.2). A stirred solution of tert-butyl 8-[[1-(2,6-dioxo-3-piperidyl)-3-methyl-2-oxo-benzimidazol-5-yl]methyl]-3,8-diazabicyclo[3.2.1]octane-3-carboxylate (180 mg, 0.372 mmol) in DCM (3 mL) was treated with TFA (1.5 mL) at room temperature. The reaction mixture was stirred for 1 hour then was concentrated and used directly without further purification. MS (ESI): m/z=384.2 [M+H]⁺.

### Preparation of 3-(3-methyl-5-(((1r,4r)-4-((methylamino)methyl)cyclohexyl)methyl)-2-oxo-2,3-dihydro-1H-benzo[d]imidazol-1-yl)piperidine-2,6-dione (A2.1)

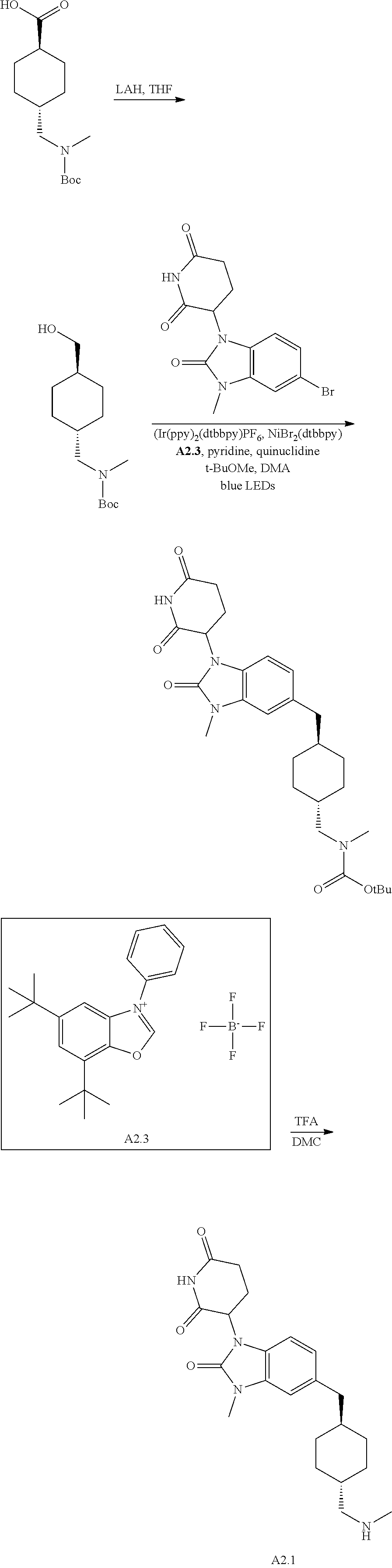

tert-butyl (((1r,4r)-4-(hydroxymethyl)cyclohexyl)methyl)(methyl)carbamate. To a solution of 4-[[tert-butoxycarbonyl(methyl)amino]methyl]cyclohexanecarboxylic acid (920 mg, 3.3 mmol) in tetrahydrofuran (15.0 mL) was added lithium aluminum hydride (2.5 mol/L, 1.36 mL, 3.3 mmol) at 0° C. under N₂. The mixture was stirred at 0° C. for 0.5 h under N₂, then sat. aq. MgSO₄ (1 mL) was added at 0° C. and followed by 1 g solid MgSO₄. The mixture was then filtered and washed with ethyl acetate (200 mL) and concentrated under reduced pressure to give the title compound. ¹H NMR (400 MHz, chloroform-d) δ 3.47 (d, J=6.1 Hz, 2H), 3.12-3.01 (m, 2H), 2.85 (s, 3H), 1.89-1.70 (m, 4H), 1.46 (s, 11H), 1.41-1.31 (m, 1H), 1.05-0.84 (m, 4H).

tert-butyl ((trans-4-((1-(2,6-dioxopiperidin-3-yl)-3-methyl-2-oxo-2,3-dihydro-1H-benzo[d]imidazol-5-yl)methyl)cyclohexyl)methyl)(methyl)carbamate. Tert-butyl N-[[4-(hydroxymethyl)cyclohexyl]methyl]-N-methyl-carbamate (0.6 g, 2.5 mmol) and 5,7-Di-tert-butyl-3-phenylbenzo[d]oxazol-3-ium tetrafluoroborate (A2.3, 0.9 g, 2.3 mmol) in MTBE (4.00 mL) was stirred at 20° C. for 5 min under N₂ to give mixture 1. Pyridine (0.18 g, 2.3 mmol) in MTBE (2.00 mL) was added dropwise to the mixture 1 and stirred at 20° C. for 10 min under N₂ and was then filtered to give mixture 2. The solution 3-(5-bromo-3-methyl-2-oxo-benzimidazol-1-yl)piperidine-2,6-dione (500 mg, 1.4 mmol), (Ir(ppy)₂(dtbbpy)PF₆ (0.02 g, 0.02 mmol), NiBr₂(dtbbpy) (0.03 g, 0.07 mmol), quinuclidine (0.28 g, 2.5 mmol) in DMA (6.00 mL) as mixture 3. Mixture 2 was added to mixture 3 and stirred at 20° C. for 12 hr under Argon and blue LEDs (450 nm). The reaction mixture was diluted with H₂O (20 mL) and extracted with ethyl acetate (10 mL×3). The combined organic layers were washed with brine (10 mL×3), dried over Na₂SO₄, filtered and concentrated under reduced pressure to give a residue. The residue was purified by reverse-phase HPLC (TFA) to give the title compound. ¹H NMR (400 MHz, Chloroform-d) δ 8.07 (s, 1H), 6.88 (d, J=8.1 Hz, 1H), 6.84 (s, 1H), 6.74 (d, J=7.9 Hz, 1H), 5.31-5.22 (m, 1H), 3.46 (s, 3H), 3.06 (br d, J=7.0 Hz, 2H), 3.02-2.94 (m, 1H), 2.89-2.84 (m, 3H), 2.82 (br s, 1H), 2.56 (br d, J=7.0 Hz, 2H), 2.32-2.21 (m, 1H), 1.80-1.64 (m, 4H), 1.62-1.42 (m, 12H), 1.04-0.82 (m, 4H). MS (ESI): m/z=443.3 [M-tBu+H]⁺.

3-(3-methyl-5-(((1r,4r)-4-((methylamino)methyl)cyclohexyl)methyl)-2-oxo-2,3-dihydro-1H-benzo[d]imidazol-1-yl)piperidine-2,6-dione (A2.1). tert-butyl N-[[4-[[1-(2,6-dioxo-3-piperidyl)-3-methyl-2-oxo-benzimidazol-5-yl]methyl]cyclohexyl]methyl]-N-methyl-carbamate (50.0 mg, 0.100 mmol) was charged to a vial equipped with a stir bar and diluted in 1 mL of DCM. TFA (0.15 mL, 2.01 mmol) was added and the mixture was stirred at room temperature for 4 hours. After 4 hours the mixture was concentrated under reduced pressure. The residue was diluted with 5 mL of toluene and further concentrated under reduced pressure to afford 3-(3-methyl-5-(((1r,4r)-4-((methylamino)methyl)cyclohexyl)methyl)-2-oxo-2,3-dihydro-1H-benzo[d]imidazol-1-yl)piperidine-2,6-dione which was used without further purification. ES/MS: m/z=399.0 [M+H]⁺.

### Preparation of 3-(5-(((1r,4r)-4-(aminomethyl)cyclohexyl)methyl)-3-methyl-2-oxo-2,3-dihydro-1H-benzo[d]imidazol-1-yl)piperidine-2,6-dione (A2.2)

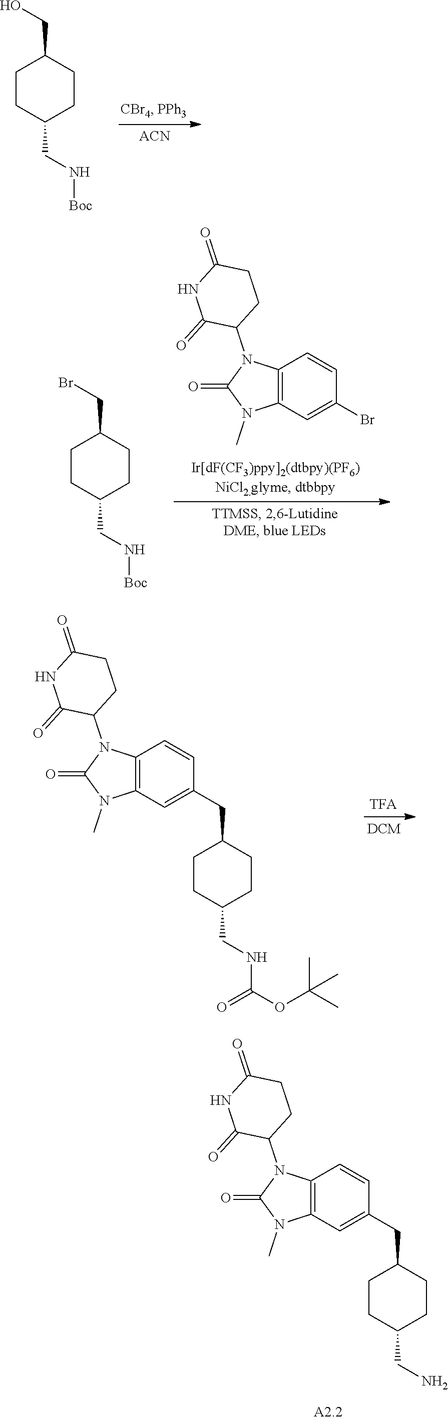

tert-butyl (((1r,4r)-4-(bromomethyl)cyclohexyl)methyl)carbamate. To a mixture of tert-butyl N-[[4-(hydroxymethyl)cyclohexyl]methyl]carbamate (2 g, 8.2 mmol, 1 eq) in ACN (20 mL) was added CBr₄ (5.4 g, 16.4 mmol) and PPh₃ (4.3 g, 16.4 mmol). The mixture was stirred at 90° C. for 7 hrs. The mixture was filtered and the filtrate was concentrated under reduced pressure to give a residue. The residue was purified by purified by flash silica gel chromatography (0-20% petroleum ether/EtOAc gradient) to give tert-butyl (((1r,4r)-4-(bromomethyl)cyclohexyl)methyl)carbamate.

tert-butyl (((1r,4r)-4-((1-(2,6-dioxopiperidin-3-yl)-3-methyl-2-oxo-2,3-dihydro-1H-benzo[d]imidazol-5-yl)methyl)cyclohexyl)methyl)carbamate. To a mixture of tert-butyl (((1r,4r)-4-(bromomethyl)cyclohexyl)methyl)carbamate (1.6 g, 5.36 mmol) and 3-(5-bromo-3-methyl-2-oxo-benzimidazol-1-yl)piperidine-2,6-dione (1.8 g, 5.36 mmol) in 1,2-dimethoxyethane (16.0 mL) was added Ir[dF(CF₃)ppy]₂(dtbpy)(PF₆) (60.1 mg, 0.054 mmol), NiCl₂·glyme (5.8 mg, 0.027 mmol), dtbbpy (7.1 mg, 0.027 mmol), tris(trimethylsilyl)silane (TTMSS; 1.3 g, 5.36 mmol) and 2,6-Lutidine (1.1 g, 10.3 mmol). The mixture was stirred and irradiated with a 34 W blue LED lamp at 25° C. for 12 hrs. The mixture was then filtered with ethyl acetate (80 mL) and the filtrate was washed with brine (20 mL×3), dried over Na₂SO₄, filtered, and concentrated under reduced pressure to give a residue. The residue was passed through a syringe filter and the filtrate was purified by prep-HPLC (TFA condition, column: Phenomenex luna C18 (250*70 mm, 15 um); mobile phase: [A: H₂O (0.1% TFA); B: ACN]; B %: 45.00%-75.00%, 20.00 min) to give tert-butyl (((1r,4r)-4-((1-(2,6-dioxopiperidin-3-yl)-3-methyl-2-oxo-2,3-dihydro-1H-benzo[d]imidazol-5-yl)methyl)cyclohexyl)methyl)carbamate. ¹H NMR (400 MHz, DMSO-d₆) δ 11.08 (s, 1H), 7.02-6.94 (m, 2H), 6.81 (d, J=9.0 Hz, 1H), 6.76 (br t, J=5.7 Hz, 1H), 5.33 (dd, J=5.3, 12.7 Hz, 1H), 3.32 (s, 3H), 3.01-2.82 (m, 1H), 2.73 (br t, J=6.3 Hz, 2H), 2.71-2.65 (m, 1H), 2.65-2.56 (m, 1H), 2.49-2.41 (m, 2H), 2.05-1.93 (m, 1H), 1.65 (br d, J=10.3 Hz, 4H), 1.53-1.21 (m, 11H), 0.98-0.70 (m, 4H). MS (ESI): m/z=429.2 (M-Bu+H)⁺.

3-(5-(((1r,4r)-4-(aminomethyl)cyclohexyl)methyl)-3-methyl-2-oxo-2,3-dihydro-1H-benzo[d]imidazol-1-yl)piperidine-2,6-dione (A2.2). tert-Butyl N-[[4-[[1-(2,6-dioxo-3-piperidyl)-3-methyl-2-oxo-benzimidazol-5-yl]methyl]cyclohexyl]methyl]carbamate (50.0 mg, 0.103 mmol) was charged to a vial equipped with a stir bar and diluted in 1 mL of DCM. TFA (0.16 mL, 2.06 mmol) was added and the mixture was stirred at room temperature for 4 hours. After 4 hours the mixture was concentrated under reduced pressure and was then further diluted with 5 mL of toluene and concentrated under reduced pressure to afford 3-[5-[[4-(aminomethyl)cyclohexyl]methyl]-3-methyl-2-oxo-benzimidazol-1-yl]piperidine-2,6-dione (A2.2) which was used without further purification. ES/MS: m/z=385.3 [M+H]⁺.

### Preparation of 3-(3-methyl-2-oxo-5-(piperidin-4-yl)-2,3-dihydro-1H-benzo[d]imidazol-1-yl)piperidine-2,6-dione (A3.2)

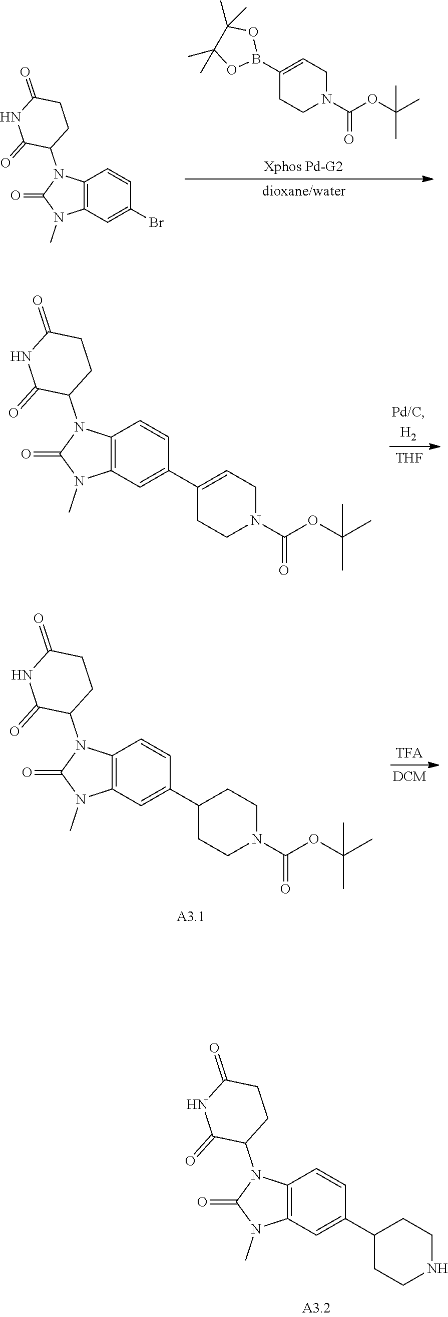

tert-butyl 4-(1-(2,6-dioxopiperidin-3-yl)-3-methyl-2-oxo-2,3-dihydro-1H-benzo[d]imidazol-5-yl)-3,6-dihydropyridine-1(2H)-carboxylate. To a solution of 3-(5-bromo-3-methyl-2-oxo-benzimidazol-1-yl)piperidine-2,6-dione (2 g, 5.9 mmol, 1 eq) in dioxane (30 mL)/H₂O (3 mL) was added tert-butyl 4-(4,4,5,5-tetramethyl-1,3,2-dioxaborolan-2-yl)-3,6-dihydro-2H-pyridine-1-carboxylate (2.7 g, 8.8 mmol, 1.5 eq), K₃PO₄ (2.5 g, 11.8 mmol, 2 eq) and Xphos Pd-G2 (465.3 mg, 591.4 μmol, 0.1 eq). The mixture was stirred at 80° C. for 1 hr under N₂. The reaction mixture was filtered and the filtrate was concentrated under reduced pressure to give a residue. The residue was dissolved in EtOAc (40 mL) and washed with H₂O (40 mL). The aqueous phase was extracted with EtOAc (40 mL×3), the combined organic layers were dried over Na₂SO₄, filtered and concentrated under reduced pressure to give a residue. The residue was triturated with EtOAc at 25° C. for 30 min, the mixture was filtered to give the title compound. ¹H NMR (400 MHz, DMSO-d₆) δ 7.28 (s, 1H), 7.11-7.01 (m, 2H), 6.12 (br s, 1H), 5.36 (dd, 1H, J=5.6, 12.8 Hz), 4.01 (br s, 2H), 3.57-3.54 (m, 2H), 3.36-3.35 (m, 4H), 3.33-3.29 (m, 1H), 2.94-2.86 (m, 1H), 2.73-2.61 (m, 2H), 2.03-1.99 (m, 1H), 1.44 (s, 9H). MS (ESI): m/z=385.2 [M-tBu+H]⁺.

tert-butyl 4-(1-(2,6-dioxopiperidin-3-yl)-3-methyl-2-oxo-2,3-dihydro-1H-benzo[d]imidazol-5-yl)piperidine-1-carboxylate (A3.1). To a bottle with Pd/C (10.0%, 0.5 g) was added tetrahydrofuran (10.0 mL) and tert-butyl 4-[1-(2,6-dioxo-3-piperidyl)-3-methyl-2-oxo-benzimidazol-5-yl]-3,6-dihydro-2H-pyridine-1-carboxylate (1.0 g, 2.2 mmol) under an H₂ atmosphere. The mixture was stirred at 20° C. for 12 hr under H₂ at 15 PSI. The reaction mixture was filtrated and the filter cake was washed with THF (3000 mL). The combined filtrate was concentrated to get a residue. The residue was triturated with MTBE (20 mL) at 25° C. for 1 hr. The residue was filtered, washed with MTBE (20 mL) and then the filter cake was dried under reduced pressure to give the title compound. ¹H NMR (400 MHz, DMSO-d₆) δ 11.09 (s, 1H), 7.11 (s, 1H), 7.01 (d, J=8.1 Hz, 1H), 6.91 (dd, J=1.1, 8.2 Hz, 1H), 5.34 (dd, J=5.4, 12.8 Hz, 1H), 4.15-4.04 (m, 2H), 3.33 (s, 3H), 2.95-2.57 (m, 6H), 2.04-1.94 (m, 1H), 1.75 (br d, J=11.9 Hz, 2H), 1.55 (dq, J=4.1, 12.5 Hz, 2H), 1.42 (s, 9H). MS (ESI): m/z=387.2 [M-Bu+H]⁺.

3-(3-methyl-2-oxo-5-(piperidin-4-yl)-2,3-dihydro-1H-benzo[d]imidazol-1-yl)piperidine-2,6-dione (A3.2): tert-butyl 4-[1-(2,6-dioxo-3-piperidyl)-3-methyl-2-oxo-benzimidazol-5-yl]piperidine-1-carboxylate (27 mg, 0.06 mmol) was suspended in HCl (4.0M in dioxane, 1 mL). After stirring for 10 minutes, the mixture was concentrated to afford 3-(3-methyl-2-oxo-5-(piperidin-4-yl)-2,3-dihydro-1H-benzo[d]imidazol-1-yl)piperidine-2,6-dione. ES/MS: m/z=342.9 [M+H]⁺.

### Preparation of tert-butyl 4-((1-(2,6-dioxopiperidin-3-yl)-3-methyl-2-oxo-2,3-dihydro-1H-benzo[d]imidazol-5-yl)methyl)piperidine-1-carboxylate (A3.3)

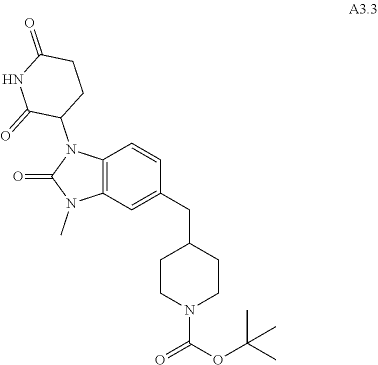 [SMILES: Cn1c(=O)n(C2CCC(=O)NC2=O)c2ccc(CC3CCN(C(=O)OC(C)(C)C)CC3)cc21]

A3.3 was prepared analogously to A3.1 using tert-butyl 4-((4,4,5,5-tetramethyl-1,3,2-dioxaborolan-2-yl)methylene)piperidine-1-carboxylate instead of tert-butyl 4-(4,4,5,5-tetramethyl-1,3,2-dioxaborolan-2-yl)-3,6-dihydro-2H-pyridine-1-carboxylate. ¹H NMR (400 MHz, DMSO-d₆) δ 11.08 (s, 1H), 7.03-6.96 (m, 2H), 6.83 (d, J=8.0 Hz, 1H), 5.33 (dd, J=5.3, 12.6 Hz, 1H), 3.91 (br d, J=12.0 Hz, 2H), 3.32 (s, 3H), 2.96-2.83 (m, 1H), 2.75-2.58 (m, 4H), 2.53 (br d, J=7.0 Hz, 2H), 2.04-1.96 (m, 1H), 1.72-1.61 (m, 1H), 1.56 (br d, J=13.1 Hz, 2H), 1.38 (s, 9H), 1.03 (dq, J=3.8, 12.2 Hz, 2H). MS (ESI): m/z=401.2 [M-Bu+H]⁺.

### Preparation of 3-(3-methyl-2-oxo-5-(piperidin-4-ylmethyl)-2,3-dihydro-1H-benzo[d]imidazol-1-yl)piperidine-2,6-dione (A3.4)

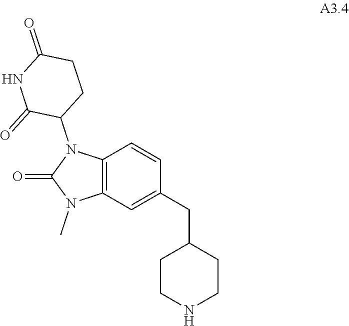 [SMILES: Cn1c(=O)n(C2CCC(=O)NC2=O)c2ccc(CC3CCNCC3)cc21]

A3.4 was prepared analogously to A3.2 using A3.3 instead of A3.1. ES/MS: m/z=357.1 [M+H]⁺.

### Preparation of 3-(5-([1,4′-bipiperidin]-4-yl)-3-methyl-2-oxo-2,3-dihydro-1H-benzo[d]imidazol-1-yl)piperidine-2,6-dione (A3.5)

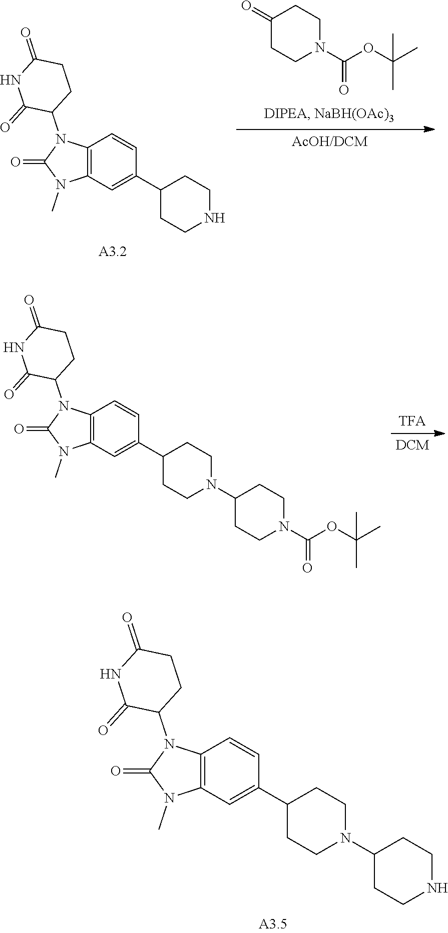 [SMILES: Cn1c(=O)n(C2CCC(=O)NC2=O)c2ccc(C3CCNCC3)cc21 ; CC(C)(C)OC(=O)N1CCC(=O)CC1 ; Cn1c(=O)n(C2CCC(=O)NC2=O)c2ccc(C3CCN(C4CCN(C(=O)OC(C)(C)C)CC4)CC3)cc21 ; Cn1c(=O)n(C2CCC(=O)NC2=O)c2ccc(C3CCN(C4CCNCC4)CC3)cc21]

tert-butyl 4-(1-(2,6-dioxopiperidin-3-yl)-3-methyl-2-oxo-2,3-dihydro-1H-benzo[d]imidazol-5-yl)-[1,4′-bipiperidine]-1′-carboxylate. To a mixture of A3.2 (0.500 g, 0.00110 mol) and tert-butyl 4-oxopiperidine-1-carboxylate (0.327 g, 0.00164 mol) in dichloromethane (4.00 mL) was added N,N-diisopropylethylamine (0.142 g, 0.00110 mol), the mixture was adjusted pH=5 with AcOH, the mixture was stirred at 25° C. for 1 hr, then the NaBH(OAc)₃ (0.348 g, 0.00164 mol) was added, the mixture was stirred at 25° C. for 12 hrs. The reaction mixture was quenched by addition NaHCO₃ (1 M) solution (5 mL) at 0° C., and then extracted with DCM (5 mL×3). The combined organic layers were dried over Na₂SO₄, filtered and concentrated under reduced pressure to give a residue. The crude product was triturated with EtOAc at 25° C. for 5 mins. Then the mixture was filtered and collected filter cake to give the title compound. MS: m/z=526.3 [M+H]⁺.

3-(5-([1,4′-bipiperidin]-4-yl)-3-methyl-2-oxo-2,3-dihydro-1H-benzo[d]imidazol-1-yl)piperidine-2,6-dione (A3.5). To the mixture of tert-butyl 4-(1-(2,6-dioxopiperidin-3-yl)-3-methyl-2-oxo-2,3-dihydro-1H-benzo[d]imidazol-5-yl)-[1,4′-bipiperidine]-1′-carboxylate (0.300 g, 0.000571 mol) in dichloromethane (3.00 mL) was added trifluoroacetic acid (1.00 mL), the mixture was stirred at 25° C. for 0.5 hr. The reaction mixture was concentrated under reduced pressure to give a residue. The residue was purified by prep-HPLC (TFA) to give the title compound. 1H NMR (400 MHz, CD₃OD) 6 ppm 7.09-7.03 (m, 3H), 5.33 (dd, 1H, J=5.6, 12.4 Hz), 3.72-3.62 (m, 5H), 3.43 (s, 3H), 3.28-3.22 (m, 2H), 3.17-3.11 (m, 2H), 2.99-2.88 (m, 2H), 2.85-2.77 (m, 2H), 2.44 (br d, 2H, J=13.2 Hz), 2.20-2.01 (m, 7H). MS: m/z=426.3[M+H]⁺.

### Preparation of 3-(3-methyl-2-oxo-4-(piperidin-4-ylmethyl)-2,3-dihydro-1H-benzo[d]imidazol-1-yl)piperidine-2,6-dione (A3.6)

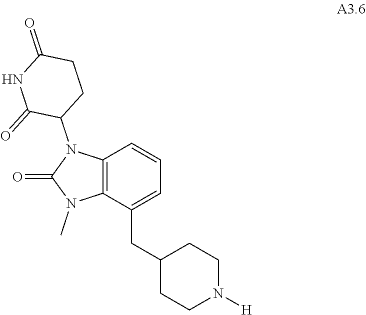 [SMILES: Cn1c(=O)n(C2CCC(=O)NC2=O)c2cccc(CC3CCNCC3)c21]

3-(3-methyl-2-oxo-4-(piperidin-4-ylmethyl)-2,3-dihydro-1H-benzo[d]imidazol-1-yl)piperidine-2,6-dione (A3.6). A3.6 was prepared analogously to A3.4 using 3-(4-bromo-3-methyl-2-oxo-2,3-dihydro-1H-benzo[d]imidazol-1-yl)piperidine-2,6-dione and tert-butyl 4-((4,4,5,5-tetramethyl-1,3,2-dioxaborolan-2-yl)methylene)piperidine-1-carboxylate as starting materials. MS: m/z=357.2 [M+H]⁺.

### Preparation of 3-(5-([4,4′-bipiperidin]-1-yl)-3-methyl-2-oxo-2,3-dihydro-1H-benzo[d]imidazol-1-yl)piperidine-2,6-dione (A.4.1)

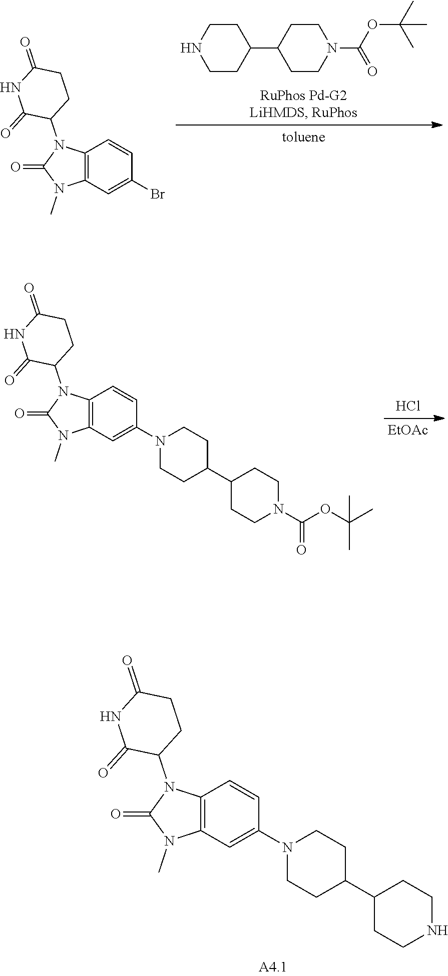

tert-butyl 1′-(1-(2,6-dioxopiperidin-3-yl)-3-methyl-2-oxo-2,3-dihydro-1H-benzo[d]imidazol-5-yl)-[4,4′-bipiperidine]-1-carboxylate. To a mixture of 3-(5-bromo-3-methyl-2-oxo-benzimidazol-1-yl)piperidine-2,6-dione (0.800 g, 0.00237 mol) and tert-butyl 4-(4-piperidyl)piperidine-1-carboxylate (0.762 g, 0.00284 mol) in toluene (40.00 mL) was added lithium bis(trimethylsilyl)amide (1.00 mol/L, 14.2 mL, 0.0142 mol) and 2-dicyclohexylphosphino-2′,6′-di-1-propoxy-1,1′-biphenyl (0.221 g, 0.000473 mol) and [2-(2-aminophenyl)phenyl]-hydroxyoxo-palladium; [2-chloro-6-(2,6-diisopropoxyphenyl)phenyl]-dicyclohexyl-phosphane (0.383 g, 0.000473 mol) and the mixture was stirred at 100° C. for 1 hr under N₂. The reaction mixture was quenched by addition of formic acid at 0° C. to adjust pH=7, and then filtered and concentrated under reduced pressure to give a residue. The residue was purified by prep-HPLC (TFA) to give the title compound. MS: m/z=526.4[M+H]⁺.

3-(5-([4,4′-bipiperidin]-1-yl)-3-methyl-2-oxo-2,3-dihydro-1H-benzo[d]imidazol-1-yl)piperidine-2,6-dione (A.4.1). To the mixture of tert-butyl 1′-(1-(2,6-dioxopiperidin-3-yl)-3-methyl-2-oxo-2,3-dihydro-1H-benzo[d]imidazol-5-yl)-[4,4′-bipiperidine]-1-carboxylate (0.400 g, 0.000761 mol) in ethyl acetate (3.00 mL) was added HCl (1.00 mL, 4 M), the mixture was stirred at 25° C. for 0.5 hr. The mixture was concentrated under reduced pressure to give a residue. The residue was purified by prep-HPLC (TFA) to give the title compound. 1H NMR (400 MHz, DMSO-d6) δ ppm 11.13 (s, 1H), 9.23-9.09 (m, 2H), 7.93 (s, 1H), 7.70 (dd, 1H, J=1.2, 8.4 Hz), 7.33 (br d, 1H, J=8.8 Hz), 5.49 (dd, 1H, J=5.2, 12.8 Hz), 3.67 (br s, 2H), 3.51 (br d, 2H, J=9.6 Hz), 3.36 (s, 3H), 3.25 (br d, 2H, J=11.2 Hz), 2.93-2.89 (m, 1H), 2.86-2.70 (m, 3H), 2.62 (br d, 1H, J=16.8 Hz), 2.05-2.02 (m, 3H), 1.91-1.81 (m, 4H), 1.55-1.46 (m, 4H). MS: m/z=426.2 [M+H]⁺.

### Preparation of 3-(3-methyl-2-oxo-5-(4-(piperidin-4-yloxy)piperidin-1-yl)-2,3-dihydro-1H-benzo[d]imidazol-1-yl)piperidine-2,6-dione (A4.2)

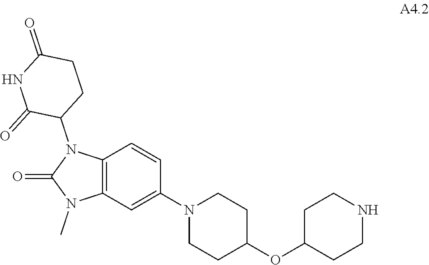 [SMILES: Cn1c(=O)n(C2CCC(=O)NC2=O)c2ccc(N3CCC(OC4CCNCC4)CC3)cc21]

A4.2 was prepared analogously to A4.1 using tert-butyl 4-(piperidin-4-yloxy)piperidine-1-carboxylate instead of tert-butyl 4-(4-piperidyl)piperidine-1-carboxylate. 1H NMR (400 MHz, DMSO-d6) δ ppm 11.15 (s, 1H), 9.26 (br s, 2H), 7.87 (br d, 1H, J=1.2 Hz), 7.68 (br d, 1H, J=8.4 Hz), 7.33 (br d, 1H, J=8.8 Hz), 5.49 (br dd, 1H, J=5.2, 12.8 Hz), 3.89-3.55 (m, 6H), 3.37 (s, 3H), 3.16 (br s, 2H), 2.96-2.89 (m, 3H), 2.74-2.60 (m, 2H), 2.16 (br s, 3H), 2.08-1.91 (m, 4H), 1.76 (br s, 2H). MS (254 nm): m/z=442.2[M+H]⁺.

### Preparation of 3-(3-methyl-2-oxo-5-(4-(piperidin-4-ylmethyl)piperidin-1-yl)-2,3-dihydro-1H-benzo[d]imidazol-1-yl)piperidine-2,6-dione (A4.3)

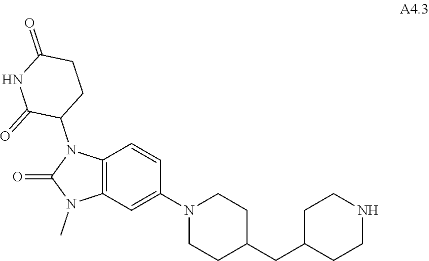 [SMILES: Cn1c(=O)n(C2CCC(=O)NC2=O)c2ccc(N3CCC(CC4CCNCC4)CC3)cc21]

A4.3 was prepared analogously to A4.1 using tert-butyl 4-(piperidin-4-ylmethyl)piperidine-1-carboxylate instead of tert-butyl 4-(4-piperidyl)piperidine-1-carboxylate. 1H NMR (400 MHz, DMSO-d6) δ ppm 11.13 (s, 1H), 9.16-8.97 (m, 2H), 7.90 (d, 1H, J=1.2 Hz), 7.69 (br d, 1H, J=8.8 Hz), 7.31 (d, 1H, J=8.8 Hz), 5.46 (dd, 1H, J=5.6, 12.8 Hz), 3.67 (br s, 2H), 3.49 (br d, 2H, J=10.0 Hz), 3.37 (s, 3H), 3.22 (br d, 2H, J=12.0 Hz), 2.92-2.71 (m, 4H), 2.63 (br d, 1H, J=16.8 Hz), 2.04-2.01 (m, 1H), 1.90 (br s, 4H), 1.79 (br d, 3H, J=12.8 Hz), 1.67 (br s, 1H), 1.39-1.31 (m, 2H), 1.22 (br s, 2H). MS: m/z=440.2[M+H]⁺.

### Preparation of 3-(3-methyl-2-oxo-5-(piperazine-1-carbonyl)-2,3-dihydro-1H-benzo[d]imidazol-1-yl)piperidine-2,6-dione (A4.5)

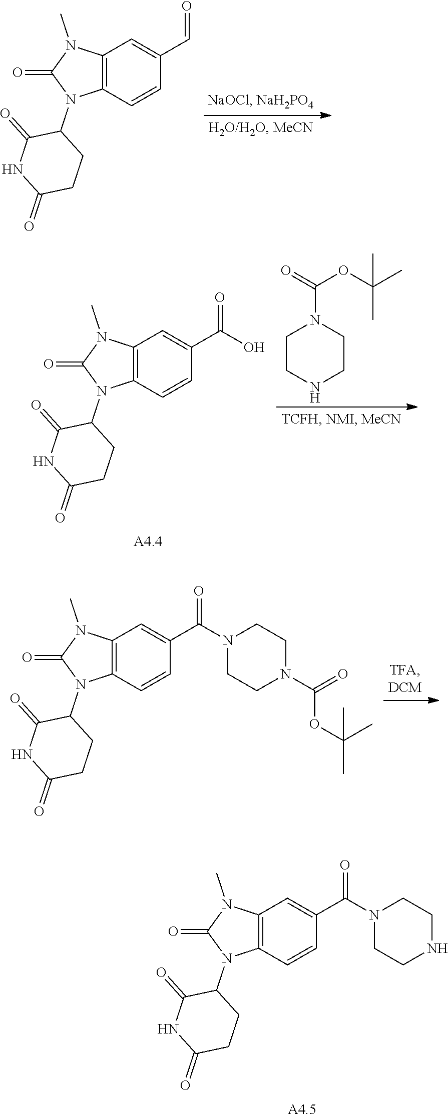

1-(2,6-dioxopiperidin-3-yl)-3-methyl-2-oxo-2,3-dihydro-1H-benzo[d]imidazole-5-carboxylic acid (A4.4). A stirred solution of 1-(2,6-dioxo-3-piperidyl)-3-methyl-2-oxo-benzimidazole-5-carbaldehyde (120 mg, 0.418 mmol) and NaH₂PO₄ (250 mg, 2.08 mmol) in acetonitrile (1.5 mL) at 0° C. was treated sequentially with H₂O₂ (30% solution, 0.08 mL, 0.835 mmol) and NaOCl (264 mg, 2.92 mmol) in water (1.5 mL). The resulting mixture was stirred for an additional hour while warming to room temperature, then was quenched by addition of saturated aqueous Na₂S₂O₃ (3 mL). The mixture was filtered and the solid was washed with acetonitrile (2 mL×3) to afford 1-(2,6-dioxo-3-piperidyl)-3-methyl-2-oxo-benzimidazole-5-carboxylic acid that was used directly without further purification. ES/MS: m/z=304.2 [M+1].

tert-butyl 4-(1-(2,6-dioxopiperidin-3-yl)-3-methyl-2-oxo-2,3-dihydro-1H-benzo[d]imidazole-5-carbonyl)piperazine-1-carboxylate. A solution of 1-(2,6-dioxo-3-piperidyl)-3-methyl-2-oxo-benzimidazole-5-carboxylic acid (65 mg, 0.215 mmol), tert-butyl piperazine-1-carboxylate (40 mg, 0.215 mmol), and 1-methylimidazole (0.55 mL, 6.87 mmol) in acetonitrile (2 mL) at room temperature was treated with chloro-N,N,N′,N′-tetramethylformamidinium hexafluorophosphate (151 mg, 0.537 mmol). The reaction mixture was stirred for an additional hour then was concentrated under vacuo, the crude residue was purified by reverse phase HPLC (0-100% acetonitrile/water/0.1% TFA) to give tert-butyl 4-[1-(2,6-dioxo-3-piperidyl)-3-methyl-2-oxo-benzimidazole-5-carbonyl]piperazine-1-carboxylate. ES/MS: m/z=472.2 [M+1].

3-(3-methyl-2-oxo-5-(piperazine-1-carbonyl)-2,3-dihydro-1H-benzo[d]imidazol-1-yl)piperidine-2,6-dione (A4.5). A stirred solution of tert-butyl 4-[1-(2,6-dioxo-3-piperidyl)-3-methyl-2-oxo-benzimidazole-5-carbonyl]piperazine-1-carboxylate (32 mg, 0.0679 mmo) in DCM (1 mL) at room temperature was treated with TFA (0.2 mL). The reaction mixture was stirred for 1 hour and was concentrated directly to afford 3-[3-methyl-2-oxo-5-(piperazine-1-carbonyl)benzimidazol-1-yl]piperidine-2,6-dione, which was used directly without further purification. ES/MS: m/z=372.1 [M+1].

### Preparation of 3-(3-methyl-2-oxo-5-(4-(piperazin-1-yl)piperidin-1-yl)-2,3-dihydro-1H-benzo[d]imidazol-1-yl)piperidine-2,6-dione (A4.6)

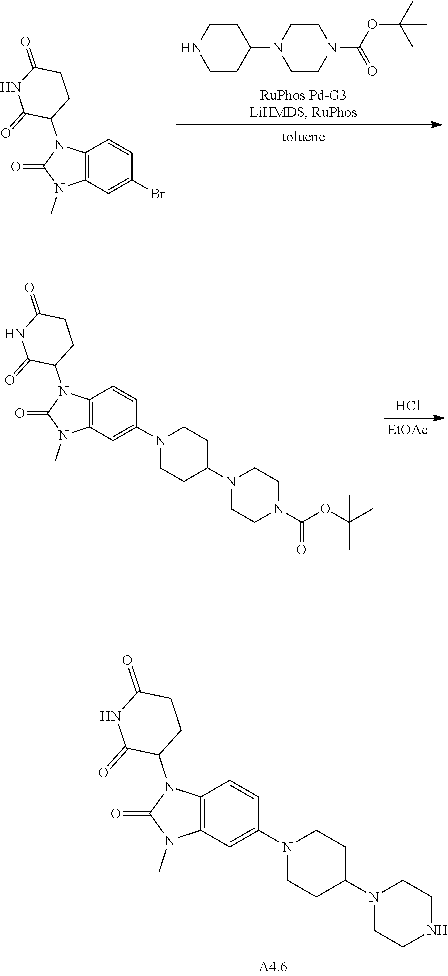

tert-butyl 4-(1-(1-(2,6-dioxopiperidin-3-yl)-3-methyl-2-oxo-2,3-dihydro-1H-benzo[d]imidazol-5-yl)piperidin-4-yl)piperazine-1-carboxylate. 3-(5-bromo-3-methyl-2-oxo-benzimidazol-1-yl)piperidine-2,6-dione (100 mg, 0.30 mmol), tert-butyl piperazine-1-carboxylate (66 mg, 0.35 mmol), RuPhos Pd G3 (25 mg, 0.030 mmol), RuPhos (13 mg, 0.030 mmol) was added to a vial with a stir bar. The vial was evacuated and backfilled with argon 3 times. 3 mL of toluene was added to the mixture. 1 M LiHMDS in THF (1.5 mL, 1.5 mmol) was added dropwise, and the mixture was heated to 80° C. for 30 min. After 30 minutes, the mixture was cooled to room temperature and 10 mL saturated aqueous NH₄Cl was added. The mixture was extracted with EtOAc (3×15 mL). The combined organics were washed with brine (10 mL), dried over Na₂SO₄, filtered and concentrated under reduced pressure. The crude material was triturated with Et₂O (˜10 mL) and filtered to afford the title compound. ES/MS: m/z=444.3 [M+1].

3-(3-methyl-2-oxo-5-(4-(piperazin-1-yl)piperidin-1-yl)-2,3-dihydro-1H-benzo[d]imidazol-1-yl)piperidine-2,6-dione (A4.6). Tert-butyl 4-[1-[1-(2,6-dioxo-3-piperidyl)-3-methyl-2-oxo-benzimidazol-5-yl]-4-piperidyl]piperazine-1-carboxylate (20 mg, 0.038 mmol) was charged to a vial equipped with a stir bar and diluted in 2 mL of DCM. 0.5 mL of TFA was added and the mixture was stirred at room temperature for 3 hours. After 3 hours the mixture was concentrated under reduced pressure to afford an oil. The oil was diluted with 2 mL of toluene and concentrated under reduced pressure to afford the title compound which was used without further purification. ES/MS: m/z=426.9 [M+1].

### Preparation of 3-(3-methyl-2-oxo-5-(piperazin-1-yl)-2,3-dihydro-1H-benzo[d]imidazol-1-yl)piperidine-2,6-dione (A4.7)

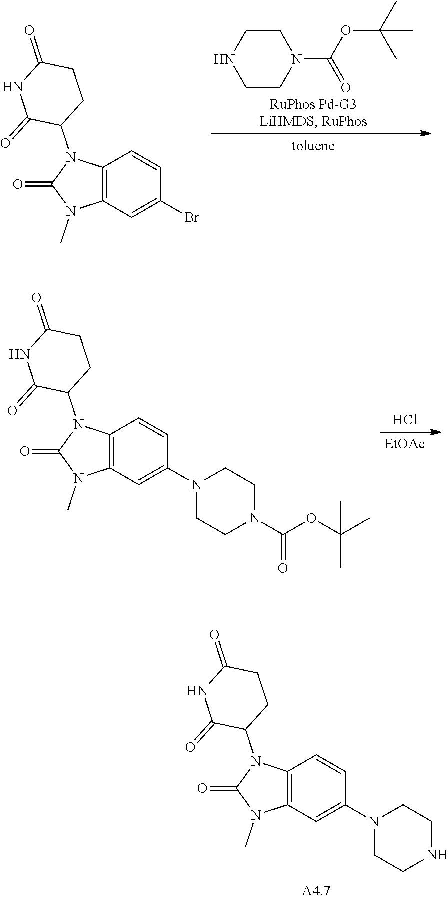

tert-butyl 4-(1-(2,6-dioxopiperidin-3-yl)-3-methyl-2-oxo-2,3-dihydro-1H-benzo[d]imidazol-5-yl)piperazine-1-carboxylate. 3-(5-bromo-3-methyl-2-oxo-benzimidazol-1-yl)piperidine-2,6-dione (100 mg, 0.30 mmol), tert-butyl piperazine-1-carboxylate (66 mg, 0.35 mmol), RuPhos Pd G3 (25 mg, 0.030 mmol), RuPhos (13 mg, 0.030 mmol) was added to a vial with a stir bar. The vial was evacuated and backfilled with argon 3 times. 3 mL of toluene was added to the mixture. 1 M LiHMDS in THF (1.5 mL, 1.5 mmol) was added dropwise, and the mixture was heated to 80° C. for 30 min. After 30 minutes, the mixture was cooled to room temperature and 10 mL saturated aqueous NH₄Cl was added. The mixture was extracted with EtOAc (3×15 mL). The combined organics were washed with brine (10 mL), dried over Na₂SO₄, filtered and concentrated under reduced pressure. The crude material was triturated with Et₂O and filtered to afford tert-butyl 4-[1-(2,6-dioxo-3-piperidyl)-3-methyl-2-oxo-benzimidazol-5-yl]piperazine-1-carboxylate. ES/MS: m/z=444.3 [M+H]⁺.

3-(3-methyl-2-oxo-5-(piperazin-1-yl)-2,3-dihydro-1H-benzo[d]imidazol-1-yl)piperidine-2,6-dione (A4.7). 3-(5-bromo-3-methyl-2-oxo-benzimidazol-1-yl)piperidine-2,6-dione (104 mg, 0.23 mmol) was charged to a vial equipped with a magnetic stir bar and diluted with 10 mL of DCM. 4.1 mL of 4M HCl in 1,4-dioxane was added and the mixture was allowed to stir for 2 h at room temperature. After 2 hours, the mixture was concentrated under reduced pressure, triturated with Et₂O and filtered to afford the 3-(3-methyl-2-oxo-5-piperazin-1-yl-benzimidazol-1-yl)piperidine-2,6-dione which was used without further purification. ES/MS: m/z=344.2 [M+H]⁺.

### Preparation of 3-(5-(3,8-diazabicyclo[3.2.1]octan-8-yl)-3-methyl-2-oxo-2,3-dihydro-1H-benzo[d]imidazol-1-yl)piperidine-2,6-dione (A4.8)

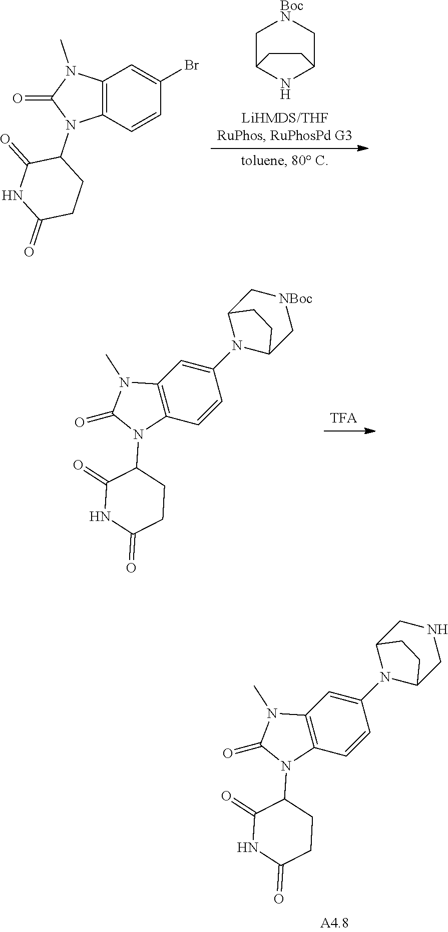 [SMILES: Cn1c(=O)n(C2CCC(=O)NC2=O)c2ccc(Br)cc21 ; CC(C)(C)OC(=O)N1CC2CCC(C1)N2 ; Cn1c(=O)n(C2CCC(=O)NC2=O)c2ccc(N3C4CCC3CN(C(=O)OC(C)(C)C)C4)cc21 ; Cn1c(=O)n(C2CCC(=O)NC2=O)c2ccc(N3C4CCC3CNC4)cc21]

tert-butyl 8-(1-(2,6-dioxopiperidin-3-yl)-3-methyl-2-oxo-2,3-dihydro-1H-benzo[d]imidazol-5-yl)-3,8-diazabicyclo[3.2.1]octane-3-carboxylate. A stirred solution of 3-(5-bromo-3-methyl-2-oxo-benzimidazol-1-yl)piperidine-2,6-dione (50 mg, 0.148 mmol), tert-butyl 3,8-diazabicyclo[3.2.1]octane-3-carboxylate (37.7 mg, 0.177 mmol), 2-Dicyclohexylphosphino-2′,6′-di-1-propoxy-1,1′-biphenyl (3.3 mg, 0.00739 mmol), (2-Dicyclohexylphosphino-2′,6′-diisopropoxy-1,1′-biphenyl)[2-(2′-amino-1,1′-biphenyl)]palladium(HJ) methanesulfonate (6.2 mg, 0.00739 mmol), and activated 4 Å molecular sieves powder (20 mg) in toluene (1.5 mL) was degassed with argon for 10 minutes, then was treated with lithium bis(trimethylsilyl)amide (1M in THF, 0.89 mL, 0.89 mmol) at room temperature. The resulting mixture was heated at 80° C. for 2 hours under argon. The reaction mixture was cooled to 0° C. and was quenched with formic acid (1 mL), then was diluted with water (10 mL) and extracted with EtOAc (10 mL×3). The combined organic layers was dried over MgSO₄, concentrated and was purified by silica flash column chromatography (0-100% EtOAc/Hexanes then 0-60% MeOH/EtOAc) to afford tert-butyl 8-[1-(2,6-dioxo-3-piperidyl)-3-methyl-2-oxo-benzimidazol-5-yl]-3,8-diazabicyclo[3.2.1]octane-3-carboxylate. ES/MS: m/z=470.2 [M+1].

3-(5-(3,8-diazabicyclo[3.2.1]octan-8-yl)-3-methyl-2-oxo-2,3-dihydro-1H-benzo[d]imidazol-1-yl)piperidine-2,6-dione (A4.8). A stirred solution of tert-butyl 8-[1-(2,6-dioxo-3-piperidyl)-3-methyl-2-oxo-benzimidazol-5-yl]-3,8-diazabicyclo[3.2.1]octane-3-carboxylate (55 mg, 0.117 mmol) in DCM (1 mL) at room temperature was treated with TFA (0.5 mL). The reaction mixture was stirred for 1 hour then concentrated in vacuo to afford 3-[5-(3,8-diazabicyclo[3.2.1]octan-8-yl)-3-methyl-2-oxo-benzimidazol-1-yl]piperidine-2,6-dione, which was used directly without further purification. ES/MS: m/z=370.2 [M+1].

### Preparation of 3-(3-methyl-2-oxo-5-(3,9-diazaspiro[5.5]undecan-3-yl)-2,3-dihydro-1H-benzo[d]imidazol-1-yl)piperidine-2,6-dione (A4.9)

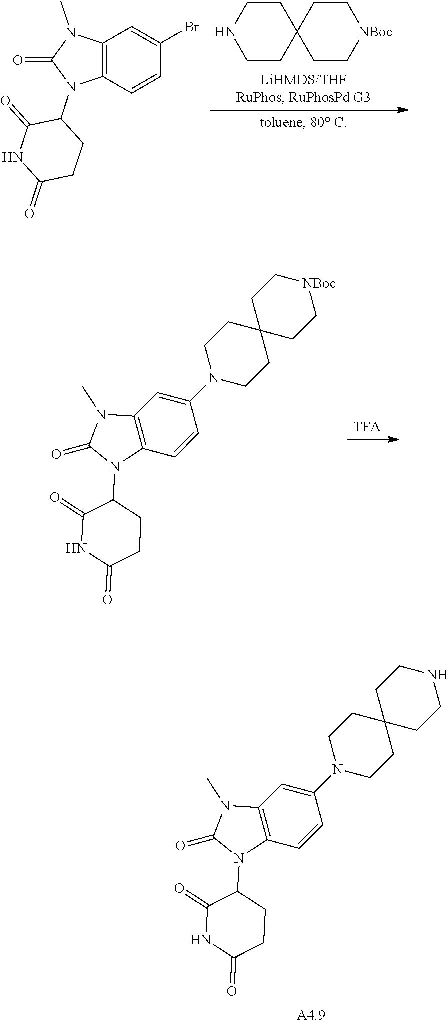

tert-butyl 9-(1-(2,6-dioxopiperidin-3-yl)-3-methyl-2-oxo-2,3-dihydro-1H-benzo[d]imidazol-5-yl)-3,9-diazaspiro[5.5]undecane-3-carboxylate. A stirred solution of 3-(5-bromo-3-methyl-2-oxo-benzimidazol-1-yl)piperidine-2,6-dione (50 mg, 0.148 mmol), tert-butyl 3,9-diazaspiro[5.5]undecane-3-carboxylate (37.7 mg, 0.177 mmol), 2-Dicyclohexylphosphino-2′,6′-di-1-propoxy-1,1′-biphenyl (3.3 mg, 0.00739 mmol), (2-Dicyclohexylphosphino-2′,6′-diisopropoxy-1,1′-biphenyl)[2-(2′-amino-1,1′-biphenyl)]palladium(II) methanesulfonate (6.2 mg, 0.00739 mmol), and activated 4 Å molecular sieves powder (20 mg) in toluene (1.5 mL) was degassed with argon for 10 minutes, then was treated with lithium bis(trimethylsilyl)amide (1M in THF, 0.89 mL, 0.89 mmol) at room temperature. The resulting mixture was heated at 80° C. for 2 hours under argon. The reaction mixture was cooled to 0° C. and was quenched with formic acid (1 mL), then was diluted with water (10 mL) and extracted with EtOAc (10 mL×3). The combined organic layers was dried over MgSO₄, concentrated and was purified by silica flash column chromatography (0-100% EtOAc/Hexanes then 0-60% MeOH/EtOAc) to afford tert-butyl 9-[1-(2,6-dioxo-3-piperidyl)-3-methyl-2-oxo-benzimidazol-5-yl]-3,9-diazaspiro[5.5]undecane-3-carboxylate. ES/MS: m/z=512.2 [M+1].

3-(3-methyl-2-oxo-5-(3,9-diazaspiro[5.5]undecan-3-yl)-2,3-dihydro-1H-benzo[d]imidazol-1-yl)piperidine-2,6-dione (A4.9). A stirred solution tert-butyl 9-[1-(2,6-dioxo-3-piperidyl)-3-methyl-2-oxo-benzimidazol-5-yl]-3,9-diazaspiro[5.5]undecane-3-carboxylate (39 mg, 0.0.0762 mmol) in DCM (1 mL) at room temperature was treated with TFA (0.5 mL). The reaction mixture was stirred for 1 hour then concentrated in vacuo to afford 3-[5-(3,9-diazaspiro[5.5]undecan-3-yl)-3-methyl-2-oxo-benzimidazol-1-yl]piperidine-2,6-dione, which was used directly without further purification. ES/MS: m/z=412.2 [M+1].

### Preparation of 3-(3-methyl-2-oxo-5-(piperazin-1-ylmethyl)-2,3-dihydro-1H-benzo[d]imidazol-1-yl)piperidine-2,6-dione (A4.10)

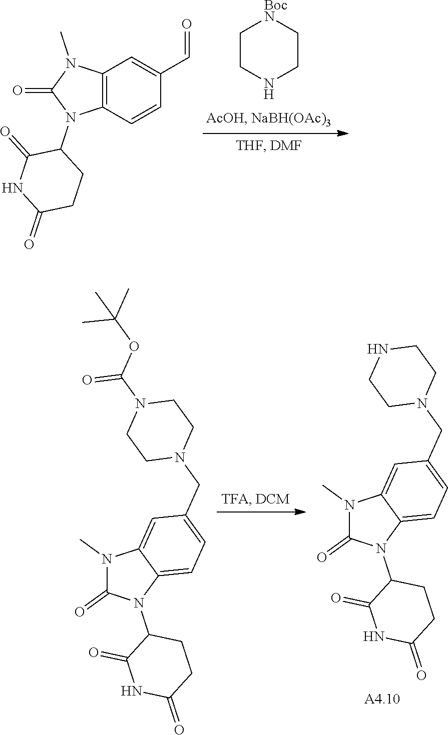 [SMILES: Cn1c(=O)n(C2CCC(=O)NC2=O)c2ccc(C=O)cc21 ; CC(C)(C)OC(=O)N1CCNCC1 ; Cn1c(=O)n(C2CCC(=O)NC2=O)c2ccc(CN3CCN(C(=O)OC(C)(C)C)CC3)cc21 ; Cn1c(=O)n(C2CCC(=O)NC2=O)c2ccc(CN3CCNCC3)cc21]

tert-butyl 4-((1-(2,6-dioxopiperidin-3-yl)-3-methyl-2-oxo-2,3-dihydro-1H-benzo[d]imidazol-5-yl)methyl)piperazine-1-carboxylate. A stirred solution of 1-(2,6-dioxo-3-piperidyl)-3-methyl-2-oxo-benzimidazole-5-carbaldehyde (100 mg, 0.348 mmol), tert-butyl piperazine-1-carboxylate (130 mg, 0.696 mmol), acetic acid (0.06 mL, 1.04 mmol) in THF (1 mL) and DMF (1 mL) was treated with sodium triacetoxyborohydride (221 mg, 1.04 mmol) at room temperature. The reaction mixture was stirred for 16 h then was basified with saturated aqueous sodium bicarbonate. The mixture was extracted with ethyl acetate (3×5 mL), and the combined organic layers were washed with water (3×10 mL), dried over MgSO4, and concentrated. The crude residue was purified by silica FCC (0-20% MeOH/DCM) to afford tert-butyl 4-[[1-(2,6-dioxo-3-piperidyl)-3-methyl-2-oxo-benzimidazol-5-yl]methyl]piperazine-1-carboxylate. ES/MS: m/z=458.2 [M+1].

3-(3-methyl-2-oxo-5-(piperazin-1-ylmethyl)-2,3-dihydro-1H-benzo[d]imidazol-1-yl)piperidine-2,6-dione (A4.10). A stirred solution of tert-butyl 4-[[1-(2,6-dioxo-3-piperidyl)-3-methyl-2-oxo-benzimidazol-5-yl]methyl]piperazine-1-carboxylate (130 mg, 0.284 mmol) in DCM (2 mL) was treated with TFA (1 mL) at room temperature. The reaction mixture was stirred for 1 hour then was concentrated to afford 3-[3-methyl-2-oxo-5-(piperazin-1-ylmethyl)benzimidazol-1-yl]piperidine-2,6-dione, which was used directly without further purification. ES/MS: m/z=358.2 [M+1].

### Preparation of 2-(1-(2,6-dioxopiperidin-3-yl)-3-methyl-2-oxo-2,3-dihydro-1H-benzo[d]imidazol-5-yl)acetaldehyde (A5.1)

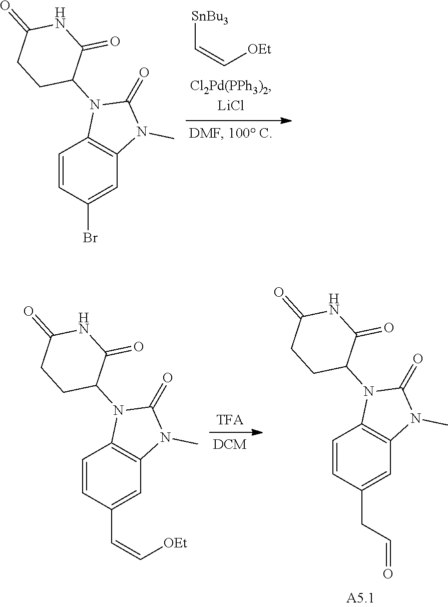

(Z)-3-(5-(2-ethoxyvinyl)-3-methyl-2-oxo-2,3-dihydro-1H-benzo[d]imidazol-1-yl)piperidine-2,6-dione. tributyl-[(Z)-2-ethoxyvinyl]stannane (0.34 mL, 1.03 mmol) was added to a mixture of 3-(5-bromo-3-methyl-2-oxo-benzimidazol-1-yl)piperidine-2,6-dione (315 mg, 0.93 mmol), Cl₂Pd(PPh₃)₂ (65 mg, 0.09 mmol), and LiCl (160 mg, 3.7 mmol) in DMF (4.0 mL). The mixture was heated to 100° C. overnight, then was diluted with EtOAc and washed with 10% aq. LiCl. The organic layer was dried over MgSO4, filtered, and concentrated. Purification by silica gel flash column chromatography afforded (Z)-3-(5-(2-ethoxyvinyl)-3-methyl-2-oxo-2,3-dihydro-1H-benzo[d]imidazol-1-yl)piperidine-2,6-dione. ES/MS: m/z=329.9 [M+H]⁺.

2-(1-(2,6-dioxopiperidin-3-yl)-3-methyl-2-oxo-2,3-dihydro-1H-benzo[d]imidazol-5-yl)acetaldehyde (A5.1). (Z)-3-(5-(2-ethoxyvinyl)-3-methyl-2-oxo-2,3-dihydro-1H-benzo[d]imidazol-1-yl)piperidine-2,6-dione (143 mg, 0.43 mmol) was suspended in DCM/TFA (4:1, 2.0 mL). After stirring for 1 hour, the mixture was concentrated and purified by silica gel flash column chromatography to afford 2-(1-(2,6-dioxopiperidin-3-yl)-3-methyl-2-oxo-2,3-dihydro-1H-benzo[d]imidazol-5-yl)acetaldehyde. ES/MS: m/z=301.9 [M+H]⁺.

### Preparation of 3-((1r,4r)-4-((1-(2,6-dioxopiperidin-3-yl)-3-methyl-2-oxo-2,3-dihydro-1H-benzo[d]imidazol-5-yl)methyl)cyclohexyl)propanoic acid (A6.1)

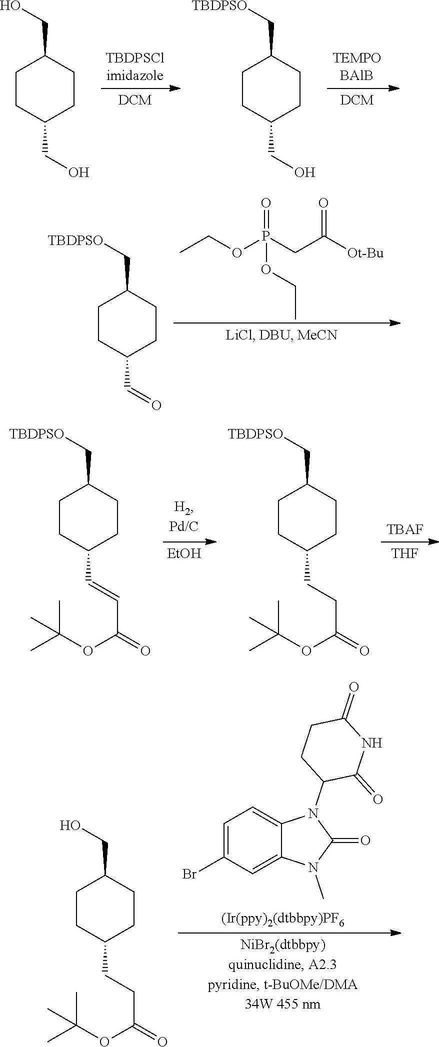

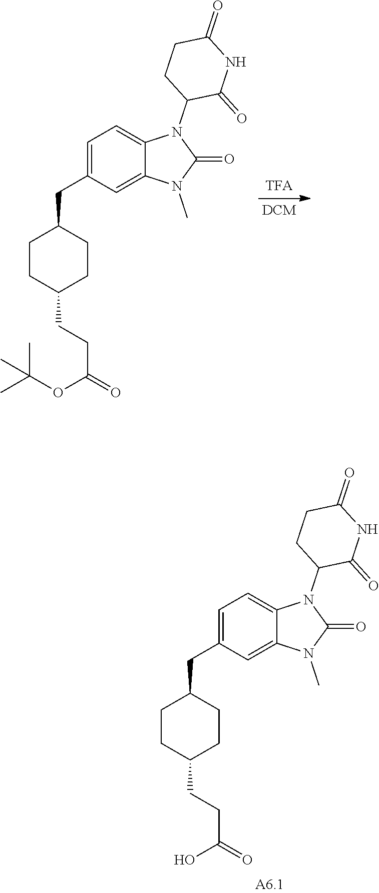 [SMILES: Cn1c(=O)n(C2CCC(=O)NC2=O)c2ccc(C[C@H]3CC[C@H](CCC(=O)OC(C)(C)C)CC3)cc21 ; Cn1c(=O)n(C2CCC(=O)NC2=O)c2ccc(C[C@H]3CC[C@H](CCC(=O)O)CC3)cc21]

(1r,4r)-4-(((tert-butyldiphenylsilyl)oxy)methyl)cyclohexyl)methanol. To a solution of [4-(hydroxymethyl)cyclohexyl]methanol (5.0 g, 34.7 mmol) in DMF (80.0 mL) at 0° C. was added imidazole (2.3 g, 34.7 mmol) and tert-butylchlorodiphenylsilane (9.5 g, 4.7 mol). The mixture was stirred at 20° C. for 2 hr under N₂. The reaction mixture was diluted with ethyl acetate 100 mL and washed with brine (100 mL×3), dried over Na₂SO₄, filtered and concentrated under reduced pressure to give a residue. The residue was purified by flash silica gel chromatography (20 g Flash Silica Column, Eluent of 0-15% Ethyl acetate/Petroleum ether gradient @60 mL/min) to give (1r,4r)-4-(((tert-butyldiphenylsilyl)oxy)methyl)cyclohexyl)methanol. ¹H NMR (400 MHz, Chloroform-d) δ 7.71-7.65 (m, 4H), 7.46-7.36 (m, 6H), 3.48 (t, J=6.0 Hz, 4H), 1.94-1.76 (m, 4H), 1.61-1.40 (m, 2H), 1.06 (s, 9H), 1.04-0.94 (m, 4H).

(1r,4r)-4-(((tert-butyldiphenylsilyl)oxy)methyl)cyclohexane-1-carbaldehyde. To a solution of (1r,4r)-4-(((tert-butyldiphenylsilyl)oxy)methyl)cyclohexyl)methanol (6.5 g, 17.0 mmol) in dichloromethane (100.0 mL) was added 2,2,6,6-Tetramethyl-1-piperidinyloxy (TEMPO) (0.529 g, 1.7 mmol) and Iodobenzene diacetate, 98% (10.9 g, 34.0 mol) at 20° C. The mixture was stirred at 20° C. for 5 hr under N₂. The reaction mixture was quenched by addition of 10% Na₂S₂O₃ 50 mL, and extracted with DCM (50 mL×3). The combined organic layers were dried over Na₂SO₄, filtered and concentrated under reduced pressure to give a residue. The residue was purified by flash silica gel chromatography (40 g Flash Silica Column, Eluent of 0-15% Ethyl acetate/Petroleum ether gradient @60 mL/min) to give (1r,4r)-4-(((tert-butyldiphenylsilyl)oxy)methyl)cyclohexane-1-carbaldehyde. ¹H NMR (400 MHz, Chloroform-d) δ 9.63 (d, J=1.3 Hz, 1H), 7.69-7.63 (m, 4H), 7.44-7.36 (m, 6H), 3.50 (d, J=6.0 Hz, 2H), 2.24-2.13 (m, 1H), 2.06-1.99 (m, 2H), 1.96-1.89 (m, 2H), 1.55-1.47 (m, 1H), 1.35-1.22 (m, 2H), 1.08-1.04 (m, 11H).

tert-butyl (E)-3-((1r,4r)-4-(((tert-butyldiphenylsilyl)oxy)methyl)cyclohexyl)acrylate. To a solution of (1r,4r)-4-(((tert-butyldiphenylsilyl)oxy)methyl)cyclohexane-1-carbaldehyde (3.2 g, 8.4 mmol) in Acetonitrile (40.0 mL) was added tert-butyl 2-diethoxyphosphorylacetate (2.5 g, 10.1 mmol), lithium chloride (0.428 g, 10.1 mmol) and 1,8-Diazabicyclo[5.4.0]undec-7-ene (1.5 g, 10.1 mmol). The mixture was stirred at 25° C. for 2 hr. The reaction mixture was diluted with H₂O 20 mL and extracted with ethyl acetate (20 mL×3). The combined organic layers were washed with brine (10 mL×3), dried over Na₂SO₄, filtered and concentrated under reduced pressure to give a residue. The residue was purified by flash silica gel chromatography (12 g Flash Silica Column, Eluent of 0-20% Ethyl acetate/Petroleum ether gradient @80 mL/min) to give tert-butyl (E)-3-((1r,4r)-4-(((tert-butyldiphenylsilyl)oxy)methyl)cyclohexyl)acrylate. ¹H NMR (400 MHz, Chloroform-d) δ 7.59 (br d, J=6.4 Hz, 4H), 7.40-7.27 (m, 6H), 6.79-6.69 (m, 1H), 5.68-5.58 (m, 1H), 3.40 (d, J=6.1 Hz, 2H), 2.05-1.91 (m, 1H), 1.76 (br t, J=13.4 Hz, 4H), 1.41 (s, 10H), 1.14-1.03 (m, 2H), 0.98 (s, 10H), 0.85-0.75 (m, 1H).

tert-butyl 3-((1r,4r)-4-(((tert-butyldiphenylsilyl)oxy)methyl)cyclohexyl)propanoate. To a bottle with Pd/C (10.0%, 1.5 g) was added EtOH (20.0 mL) and tert-butyl (E)-3-((1r,4r)-4-(((tert-butyldiphenylsilyl)oxy)methyl)cyclohexyl)acrylate (3.0 g, 6.27 mmol) in EtOH (30.0 mL) under N₂ atmosphere. The mixture was stirred at 50° C. for 12 hr under H₂ at 15 PSI. The reaction mixture was filtered, washed with 1000 mL THF and concentrated under reduced pressure to give tert-butyl 3-((1r,4r)-4-(((tert-butyldiphenylsilyl)oxy)methyl)cyclohexyl)propanoate. ¹H NMR (400 MHz, Chloroform-d) δ 7.70-7.64 (m, 4H), 7.46-7.35 (m, 6H), 3.46 (d, J=6.3 Hz, 2H), 2.23 (t, J=7.8 Hz, 2H), 1.88-1.72 (m, 4H), 1.53-1.47 (m, 3H), 1.45 (s, 9H), 1.24-1.13 (m, 1H), 1.05 (s, 9H), 1.02-0.86 (m, 4H).

tert-butyl 3-((1r,4r)-4-(((tert-butyldiphenylsilyl)oxy)methyl)cyclohexyl)propanoate. To a bottle with Pd/C (10.0%, 1.5 g) was added EtOH (20.0 mL) and tert-butyl (E)-3-((1r,4r)-4-(((tert-butyldiphenylsilyl)oxy)methyl)cyclohexyl)acrylate (3.0 g, 6.27 mmol) in EtOH (30.0 mL) under N₂ atmosphere. The mixture was stirred at 50° C. for 12 hr under H₂ at 15 PSI. The reaction mixture was filtered, washed with 1000 mL THF and concentrated under reduced pressure to to give tert-butyl 3-((1r,4r)-4-(((tert-butyldiphenylsilyl)oxy)methyl)cyclohexyl)propanoate. ¹H NMR (400 MHz, Chloroform-d) δ 7.70-7.64 (m, 4H), 7.46-7.35 (m, 6H), 3.46 (d, J=6.3 Hz, 2H), 2.23 (t, J=7.8 Hz, 2H), 1.88-1.72 (m, 4H), 1.53-1.47 (m, 3H), 1.45 (s, 9H), 1.24-1.13 (m, 1H), 1.05 (s, 9H), 1.02-0.86 (m, 4H).

tert-butyl 3-((1r,4r)-4-(hydroxymethyl)cyclohexyl)propanoate. To a solution of tert-butyl 3-[4-[[tert-butyl(diphenyl)silyl]oxymethyl]cyclohexyl]propanoate (3.0 g, 6.24 mmol) in THF (30.0 mL) was added tetrabutylammonium fluoride (1 mol/L, 6.2 mL, 6.2 mmol). The mixture was stirred at 20° C. for 2 hr The reaction mixture was diluted with NH₄Cl (20 mL) and extracted with ethyl acetate (20 mL×3). The combined organic layers were washed with brine (20 mL×3), dried over Na₂SO₄, filtered and concentrated under reduced pressure to give a residue. The residue was purified by flash silica gel chromatography (12 g Flash Silica Column, Eluent of 0-20% Ethyl acetate/Petroleum ether gradient @60 mL/min) to give tert-butyl 3-((1r,4r)-4-(hydroxymethyl)cyclohexyl)propanoate. ¹H NMR (400 MHz, DMSO-d₆) δ 4.32 (t, J=5.4 Hz, 1H), 3.18 (t, J=5.8 Hz, 2H), 2.18 (t, J=7.7 Hz, 2H), 1.78-1.64 (m, 4H), 1.43-1.34 (m, 11H), 1.30-1.21 (m, 1H), 1.16-1.05 (m, 1H), 0.91-0.74 (m, 4H).

tert-butyl 3-((1r,4r)-4-((1-(2,6-dioxopiperidin-3-yl)-3-methyl-2-oxo-2,3-dihydro-1H-benzo[d]imidazol-5-yl)methyl)cyclohexyl)propanoate. tert-butyl 3-((1r,4r)-4-(hydroxymethyl)cyclohexyl)propanoate (0.627 g, 2.59 mmol) and A2.3 (0.935 g, 2.37 mmol) in MTBE (6 mL) was stirred at 20° C. for 5 min under N₂ to give mixture 1. pyridine (0.187 g, 2.37 mmol) in MTBE (6.0 mL) was added dropwise to the mixture 1 and stirred at 20° C. for 10 min under N₂ then filtered to give a liquid as mixture 2. The 3-(5-bromo-3-methyl-2-oxo-benzimidazol-1-yl)piperidine-2,6-dione (0.5 g, 1.48 mmol), (Ir(ppy)₂(dtbbpy)PF₆ (0.0203 g, 0.022 mmol), NiBr₂(dtbbpy) (0.0360 g, 0.074 mmol), quinuclidine (0.288 g, 2.59 mmol) and in DMA (6.00 mL) as mixture 3. Mixture 2 was added to mixture 3 and stirred at 20° C. for 12 hr under Argon and blue LEDs (450 nm). The reaction mixture was diluted with H₂O 20 mL and extracted with ethyl acetate (10 mL×3). The combined organic layers were washed with brine (10 mL×3), dried over Na₂SO₄, filtered and concentrated under reduced pressure to give a residue. The residue was triturated with 20 mL MTBE at 20° C. for 20 min, filtered, washed with 20 mL MTBE and the filter cake was dried under reduced pressure to give a residue. The residue filtrate was purified by prep-HPLC (TFA condition, column: Phenomenex Luna C18 100*30 mm*5 um; mobile phase: [A: H₂O (0.1% TFA); B: ACN]; B %: 50.00%-80.00%, 9.00 min) to give tert-butyl 3-((1r,4r)-4-((1-(2,6-dioxopiperidin-3-yl)-3-methyl-2-oxo-2,3-dihydro-1H-benzo[d]imidazol-5-yl)methyl)cyclohexyl)propanoate. ¹H NMR (400 MHz, Chloroform-d) δ 8.04 (s, 1H), 6.85 (br d, J=7.9 Hz, 1H), 6.81 (s, 1H), 6.71 (d, J=7.9 Hz, 1H), 5.29-5.15 (m, 1H), 3.43 (s, 3H), 3.03-2.91 (m, 1H), 2.89-2.82 (m, 1H), 2.81-2.70 (m, 1H), 2.52 (br d, J=7.0 Hz, 2H), 2.29-2.17 (m, 3H), 1.72 (br t, J=8.9 Hz, 4H), 1.52-1.41 (m, 12H), 1.27-1.10 (m, 1H), 1.03-0.80 (m, 4H). MS (ESI): m/z=248.2 [M+H]⁺.

3-((1r,4r)-4-((1-(2,6-dioxopiperidin-3-yl)-3-methyl-2-oxo-2,3-dihydro-1H-benzo[d]imidazol-5-yl)methyl)cyclohexyl)propanoic acid (A6.1). tert-butyl 3-((1r,4r)-4-((1-(2,6-dioxopiperidin-3-yl)-3-methyl-2-oxo-2,3-dihydro-1H-benzo[d]imidazol-5-yl)methyl)cyclohexyl)propanoate (75 mg, 0.16 mmol) was charged to a vial dissolved in 3 mL DCM and 0.5 mL TFA. The mixture was stirred for 90 minutes and was the concentrated under reduced pressure. Hexane and toluene were then added to the vial and was then concentrated under reduced pressure and the material was used without further purification. ES/MS: m/z=425.9 [M−H]⁻.

### Preparation of 3-(6-Fluoro-3-methyl-2-oxo-5-(piperidin-4-ylmethyl)-2,3-dihydro-1H-benzo[d]imidazol-1-yl)piperidine-2,6-dione (A7.3)

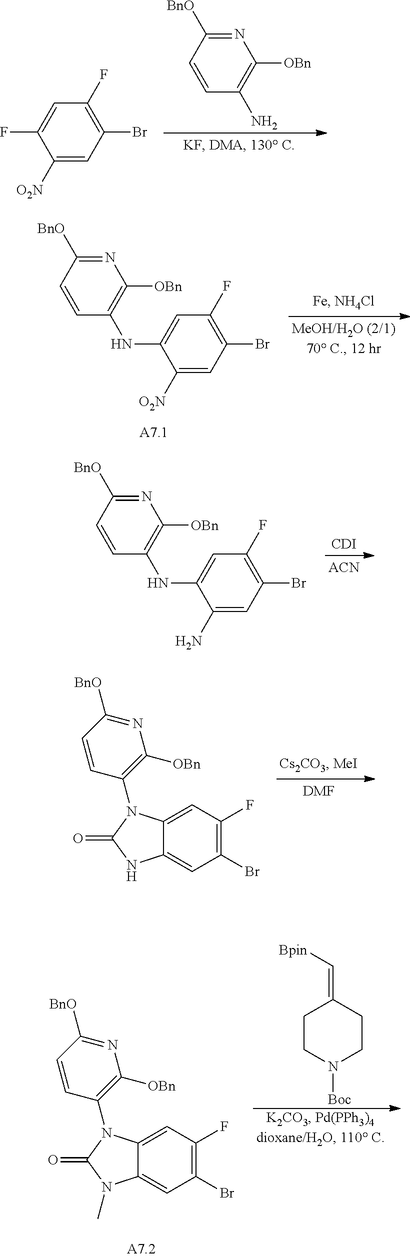

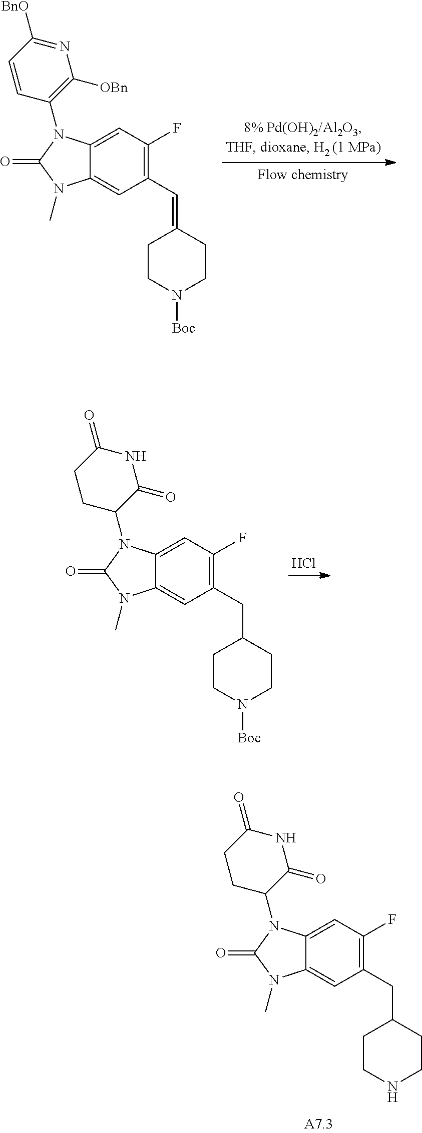 [SMILES: Cn1c(=O)n(-c2ccc(OCc3ccccc3)nc2OCc2ccccc2)c2cc(F)c(C=C3CCN(C(=O)OC(C)(C)C)CC3)cc21 ; "Flow chemistry" ; Cn1c(=O)n(C2CCC(=O)NC2=O)c2cc(F)c(CC3CCN(C(=O)OC(C)(C)C)CC3)cc21 ; Cl ; Cn1c(=O)n(C2CCC(=O)NC2=O)c2cc(F)c(CC3CCNCC3)cc21]

2,6-bis(benzyloxy)-N-(4-bromo-5-fluoro-2-nitrophenyl)pyridin-3-amine (A7.1). To a solution of 1-bromo-2,4-difluoro-5-nitro-benzene (10 g, 42.0 mmol, 1 eq) in DMA (10 mL) was added 2,6-dibenzyloxypyridin-3-amine (12.8 g, 42.0 mmol, 1 eq) and KF (2.9 g, 50.4 mmol, 1.2 eq). The mixture was stirred at 130° C. for 12 hr. The reaction mixture was diluted with H₂O 300 mL and extracted with Ethyl acetate 1200 mL (400 mL×3). The combined organic layers were washed with brine 600 mL (200 mL×3), dried over Na₂SO₄, filtered and concentrated under reduced pressure to give 2,6-bis(benzyloxy)-N-(4-bromo-5-fluoro-2-nitrophenyl)pyridin-3-amine. MS (ESI): m/z=524.1/526.0 [M+H]⁺.

N¹-(2,6-bis(benzyloxy)pyridin-3-yl)-4-bromo-5-fluorobenzene-1,2-diamine. To a solution of 2,6-bis(benzyloxy)-N-(4-bromo-5-fluoro-2-nitrophenyl)pyridin-3-amine (20.1 g, 38.4 mmol) in MeOH (264 mL)/H₂O (132 mL) was added Fe (12.9 g, 230.0 mmol) and ammonium chloride (5.1 g, 96.0 mmol). The mixture was stirred at 70° C. for 12 hr. The reaction mixture was filtered with EtOH (2000 mL) and the filtrate was concentrated under reduced pressure to give a residue. Then the residue was diluted with H₂O 400 mL and extracted with Ethyl acetate 1200 mL (400 mL×3). The combined organic layers were washed with brine 600 mL (200 mL×3), dried over Na₂SO₄, filtered and concentrated under reduced pressure to give a residue. The residue was purified by flash silica gel chromatography to give N¹-(2,6-bis(benzyloxy)pyridin-3-yl)-4-bromo-5-fluorobenzene-1,2-diamine. ¹H NMR (400 MHz, DMSO-d₆) δ7.44-7.26 (m, 1 OH), 6.85 (br d, J=5.9 Hz, 1H), 6.50 (br s, 1H), 6.42 (br d, J=7.1 Hz, 1H), 6.35 (br d, J=10.0 Hz, 1H), 5.39 (br s, 2H), 5.29 (br s, 2H), 4.88 (br s, 2H); MS (ESI): m/z=494.0/496.0 [M+H]⁺.

1-(2,6-bis(benzyloxy)pyridin-3-yl)-5-bromo-6-fluoro-1,3-dihydro-2H-benzo[d]imidazol-2-one. To a solution of N¹-(2,6-bis(benzyloxy)pyridin-3-yl)-4-bromo-5-fluorobenzene-1,2-diamine (13.6 g, 27.6 mmol) in ACN (273 mL) was added di(imidazol-1-yl)methanone (5.3 g, 33.1 mmol) at 20° C. The mixture was stirred at 25° C. for 12 hr. The reaction mixture was diluted with H₂O 100 mL and extracted with Ethyl acetate 600 mL (200 mL×3). The combined organic layers were washed with brine 600 mL (200 mL×3), dried over Na₂SO₄, filtered and concentrated under reduced pressure to give a residue. The residue was triturated with MTBE (60 mL) at 25° C. for 1 hr. The residue was filtered, washed with MTBE (50 mL) and then the filter cake was dried under reduced pressure to give 1-(2,6-bis(benzyloxy)pyridin-3-yl)-5-bromo-6-fluoro-1,3-dihydro-2H-benzo[d]imidazol-2-one. ¹H NMR (400 MHz, DMSO-d₆) δ 11.30 (s, 1H), 7.81 (d, J=8.3 Hz, 1H), 7.49-7.42 (m, 2H), 7.42-7.31 (m, 3H), 7.30-7.22 (m, 6H), 6.77 (d, J=8.8 Hz, 1H), 6.61 (d, J=8.3 Hz, 1H), 5.52-5.28 (m, 4H); MS (ESI): m/z=520.1/521.8 [M+H]⁺.

1-(2,6-bis(benzyloxy)pyridin-3-yl)-5-bromo-6-fluoro-3-methyl-1,3-dihydro-2H-benzo[d]imidazol-2-one (A7.2). To a solution of 1-(2,6-bis(benzyloxy)pyridin-3-yl)-5-bromo-6-fluoro-1,3-dihydro-2H-benzo[d]imidazol-2-one (10.3 g, 19.9 mmol) in Dimethylformamide (100 mL) was added cesium carbonate (13.0 g, 39.8 mmol) and iodomethane (5.6 g, 39.8 mmol). The mixture was stirred at 70° C. for 12 hr. The reaction mixture was diluted with H₂O 100 mL and extracted with Ethyl acetate 300 mL (100 mL×3). The combined organic layers were washed with brine 300 mL (100 mL×3), dried over Na₂SO₄, filtered and concentrated under reduced pressure to give a residue. The residue was purified by flash silica gel chromatography to give 1-(2,6-bis(benzyloxy)pyridin-3-yl)-5-bromo-6-fluoro-3-methyl-1,3-dihydro-2H-benzo[d]imidazol-2-one. ¹H NMR (400 MHz, DMSO-d₆) δ 7.81 (d, J=8.3 Hz, 1H), 7.59 (d, J=5.9 Hz, 1H), 7.47-7.42 (m, 2H), 7.42-7.32 (m, 3H), 7.27 (s, 5H), 6.85 (d, J=8.6 Hz, 1H), 6.62 (d, J=8.3 Hz, 1H), 5.49-5.29 (m, 4H), 3.38 (s, 3H); MS (ESI): m/z=534.0/535.9 [M+H]⁺.

Tert-butyl 4-((1-(2,6-bis(benzyloxy)pyridin-3-yl)-6-fluoro-3-methyl-2-oxo-2,3-dihydro-1H-benzo[d]imidazol-5-yl)methylene)piperidine-1-carboxylate. To a solution of 1-(2,6-bis(benzyloxy)pyridin-3-yl)-5-bromo-6-fluoro-3-methyl-1,3-dihydro-2H-benzo[d]imidazol-2-one (5.0 g, 9.3 mmol) in 1,4-Dioxane (100 mL)/H₂O (20.0 mL) was added tert-butyl 4-ethylidenepiperidine-1-carboxylate (2.9 g, 14.0 mmol), potassium carbonate (2.7 g, 19.6 mmol) and Tetrakis(triphenylphosphine)palladium(0) (1.7 g, 1.5 mmol). The mixture was stirred at 110° C. for 12 hr. The reaction mixture was diluted with H₂O 40 mL and extracted with Ethyl acetate 120 mL (40 mL×3). The combined organic layers were washed with brine 60 mL (20 mL×3), dried over Na₂SO₄, filtered and concentrated under reduced pressure to give a residue. The residue was purified by flash silica gel chromatography to give tert-butyl 4-((1-(2,6-bis(benzyloxy)pyridin-3-yl)-6-fluoro-3-methyl-2-oxo-2,3-dihydro-1H-benzo[d]imidazol-5-yl)methylene)piperidine-1-carboxylate. ¹H NMR (400 MHz, CHLOROFORM-d) δ 7.61 (d, J=8.4 Hz, 1H), 7.47-7.32 (m, 5H), 7.27 (s, 5H), 6.75 (d, J=5.9 Hz, 1H), 6.53 (d, J=8.3 Hz, 1H), 6.46 (d, J=9.3 Hz, 1H), 6.31 (s, 1H), 5.53-5.25 (m, 4H), 3.55 (br t, J=5.6 Hz, 2H), 3.46 (s, 3H), 3.45-3.42 (m, 1H), 2.43-2.32 (m, 4H), 1.67 (br d, J=6.3 Hz, 1H), 1.50 (s, 9H); MS (ESI): m/z=651.4 [M+H]⁺.

Tert-butyl 4-((1-(2,6-dioxopiperidin-3-yl)-6-fluoro-3-methyl-2-oxo-2,3-dihydro-1H-benzo[d]imidazol-5-yl)methyl)piperidine-1-carboxylate. Dissolve the tert-butyl 4-((1-(2,6-bis(benzyloxy)pyridin-3-yl)-6-fluoro-3-methyl-2-oxo-2,3-dihydro-1H-benzo[d]imidazol-5-yl)methylene)piperidine-1-carboxylate (4.4 g, 6.7 mmol) in THF (132 mL) and 1,4-dioxane (132 mL). The Fixed bed was packed with Pd(OH)₂/Al₂O₃ (8.0%, 2.0 g). The H₂ back pressure regulator was adjusted to 1 MPa, and the flow rate of H₂ was 30 mL/min. Then the solution S1 was pumped by Pump 1 {S1, P1, 0.4 mL/min} to fixed bed {FLR1, SS, fixed bed, 6.350 (¼″) mm, 1 mL, 50° C.}. The solution S1 was flowing through {FLR1, 2.5 mins} to leave the reactor zone, then the reaction mixture was collected from the reactor output. The fixed bed was washed by extra THF. Then the reaction mixture concentrated under reduced pressure to give a residue (2 g). Then dissolve the residue in 1,4-dioxane (60.0 mL) and THF (60.0 mL). The Fixed bed was packed with Pd(OH)₂/Al₂O₃ (8.0%, 0.5 g). The H₂ back pressure regulator was adjusted to 1 Mpa, and the flow rate of H₂ was 30 mL/min. Then the solution S1 was pumped by FLR1, SS, Fixed bed, 6.350 (¼″) mm, 1 mL, 50° C. to fixed bed FLR1, 2.5 min. The solution S1 was flowing through FLR1, 2.5 min to leave the reactor zone, then the reaction mixture was collected from the reactor output. The reaction mixture was concentrated under reduced pressure to give a residue. The residue was purified by flash silica gel chromatography, then the residue was triturated with MTBE (5 mL) at 25° C. for 1 hr. The residue was filtered, washed with MTBE (20 mL) and then the filter cake was dried under reduced to give tert-butyl 4-((1-(2,6-dioxopiperidin-3-yl)-6-fluoro-3-methyl-2-oxo-2,3-dihydro-1H-benzo[d]imidazol-5-yl)methyl)piperidine-1-carboxylate (A7.1). ¹H NMR (400 MHz, CHLOROFORM-d) δ 8.12 (s, 1H), 6.74 (d, J=6.0 Hz, 1H), 6.55 (d, J=9.3 Hz, 1H), 5.18 (dd, J=5.3, 12.8 Hz, 1H), 4.21-3.95 (m, 2H), 3.42 (s, 3H), 3.01-2.92 (m, 1H), 2.90-2.78 (m, 1H), 2.76-2.58 (m, 5H), 2.30-2.19 (m, 1H), 1.68 (br dd, J=3.5, 7.1 Hz, 1H), 1.64 (br s, 2H), 1.46 (s, 9H), 1.25-1.12 (m, 2H); MS (ESI): m/z=375.2 (MS-Boc+H)⁺.

3-(6-Fluoro-3-methyl-2-oxo-5-(piperidin-4-ylmethyl)-2,3-dihydro-1H-benzo[d]imidazol-1-yl)piperidine-2,6-dione (A7.3). Tert-butyl 4-((1-(2,6-dioxopiperidin-3-yl)-6-fluoro-3-methyl-2-oxo-2,3-dihydro-1H-benzo[d]imidazol-5-yl)methyl)piperidine-1-carboxylate (0.0500 g, 0.000105 mol) was suspended in 3 mL of 1M HCl in dioxane and allower to stir overnight. After the allotted time, the reaction mixture was concentrated to afford crude 3-(6-Fluoro-3-methyl-2-oxo-5-(piperidin-4-ylmethyl)-2,3-dihydro-1H-benzo[d]imidazol-1-yl)piperidine-2,6-dione (A7.2), which was used as is in subsequent reactions. MS (ESI): m/z=375.1 [M+H]⁺.

### Preparation of 2,6-bis(benzyloxy)-N-(4-bromo-3-fluoro-2-nitrophenyl)pyridin-3-amine (A8.1)

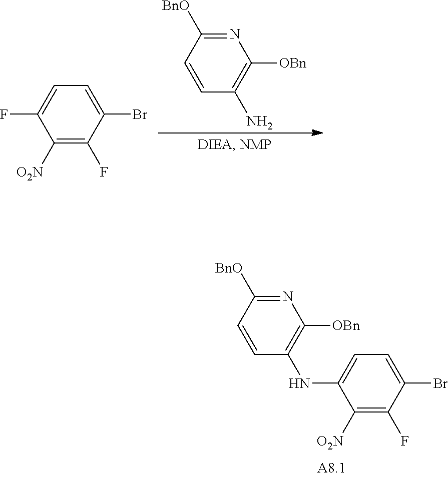 [SMILES: O=[N+]([O-])c1c(F)ccc(Br)c1F ; Nc1ccc(OCc2ccccc2)nc1OCc1ccccc1 ; O=[N+]([O-])c1c(Nc2ccc(OCc3ccccc3)nc2OCc2ccccc2)ccc(Br)c1F]

2,6-bis(benzyloxy)-N-(4-bromo-3-fluoro-2-nitrophenyl)pyridin-3-amine (A8.1). To a solution of 1-bromo-2,4-difluoro-3-nitro-benzene (9 g, 37.8 mmol, 1 eq) and 2,6-dibenzyloxypyridin-3-amine (11.5 g, 37.8 mmol, 1 eq) in NMP (32 mL) was added DIEA (4.8 g, 37.8 mmol, 6.5 mL, 1 eq). The mixture was stirred at 110° C. for 16 hr. At room temperature, H₂O (100 mL) was added the mixture was and extracted with ethyl acetate (100 mL×3). The combined organic layers were washed with NaCl (100 mL×3), dried over Na₂SO₄, filtered and concentrated under reduced pressure to give 2,6-bis(benzyloxy)-N-(4-bromo-3-fluoro-2-nitrophenyl)pyridin-3-amine, which was used crude in subsequent reactions. MS (ESI): m/z=526.0/524.0 [M+H]⁺.

### Preparation of 3-(4-fluoro-3-methyl-2-oxo-5-(piperidin-4-ylmethyl)-2,3-dihydro-1H-benzo[d]imidazol-1-yl)piperidine-2,6-dione (A8.2)

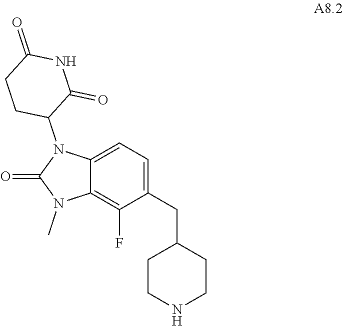 [SMILES: Cn1c(=O)n(C2CCC(=O)NC2=O)c2ccc(CC3CCNCC3)c(F)c21]

3-(4-fluoro-3-methyl-2-oxo-5-(piperidin-4-ylmethyl)-2,3-dihydro-1H-benzo[d]imidazol-1-yl)piperidine-2,6-dione (A8.2) was prepared analogously to A7.3, beginning with A8.1 instead of A7.1. MS (ESI): m/z=375.1 [M+H]⁺.

### Preparation of 3-(5-((2,7-diazaspiro[3.5]nonan-2-yl)methyl)-3-methyl-2-oxo-2,3-dihydro-1H-benzo[d]imidazol-1-yl)piperidine-2,6-dione (A9.1)

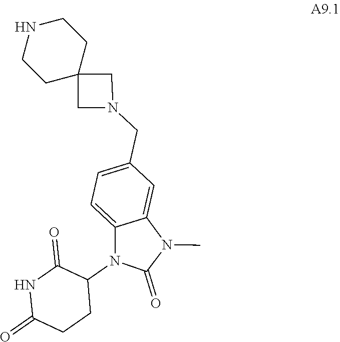 [SMILES: Cn1c(=O)n(C2CCC(=O)NC2=O)c2ccc(CN3CC4(CCNCC4)C3)cc21]

3-(5-((2,7-diazaspiro[3.5]nonan-2-yl)methyl)-3-methyl-2-oxo-2,3-dihydro-1H-benzo[d]imidazol-1-yl)piperidine-2,6-dione (A9.1). A9.1 was prepared analogously to A1.2 using A1.1 and tert-butyl 2,7-diazaspiro[3.5]nonane-7-carboxylate as starting materials. MS (ESI): m/z=398.2 [M+H]⁺.

### Preparation of 3-(5-(((1S,4S)-2,5-diazabicyclo[2.2.1]heptan-2-yl)methyl)-3-methyl-2-oxo-2,3-dihydro-1H-benzo[d]imidazol-1-yl)piperidine-2,6-dione (A10.1)

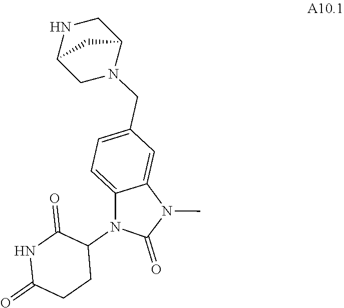

3-(5-(((1S,4S)-2,5-diazabicyclo[2.2.1]heptan-2-yl)methyl)-3-methyl-2-oxo-2,3-dihydro-1H-benzo[d]imidazol-1-yl)piperidine-2,6-dione (A10.1). A10.1 was prepared analogously to A1.2 using A1.1 and tert-butyl (1S,4S)-2,5-diazabicyclo[2.2.1]heptane-2-carboxylate as starting materials. (ESI): m/z=370.2 [M+H]⁺.

### Preparation of 3-(5-((2,7-diazaspiro[3.5]nonan-7-yl)methyl)-3-methyl-2-oxo-2,3-dihydro-1H-benzo[d]imidazol-1-yl)piperidine-2,6-dione (A11.1)

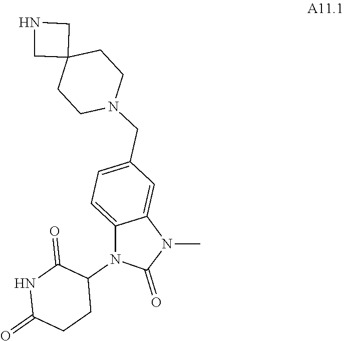 [SMILES: Cn1c(=O)n(C2CCC(=O)NC2=O)c2ccc(CN3CCC4(CC3)CNC4)cc21]

A11.1. A11.1 was prepared analogously to A1.2 using A1.1 and tert-butyl 2,7-diazaspiro[3.5]nonane-2-carboxylate as starting materials. ES/MS: m/z=398.2 [M+H]⁺.

### Preparation of 3-(5-((3,6-diazabicyclo[3.1.1]heptan-6-yl)methyl)-3-methyl-2-oxo-2,3-dihydro-1H-benzo[d]imidazol-1-yl)piperidine-2,6-dione (A12.1)

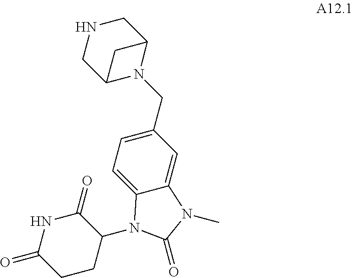 [SMILES: Cn1c(=O)n(C2CCC(=O)NC2=O)c2ccc(CN3C4CNCC3C4)cc21]

A12.1. A12.1 was prepared analogously to A1.2 using A1.1 and tert-butyl 3,6-diazabicyclo[3.1.1]heptane-3-carboxylate as starting materials. ES/MS: m/z=370.2 [M+H]⁺.

### Preparation of 3-(5-((2,6-diazaspiro[3.4]octan-2-yl)methyl)-3-methyl-2-oxo-2,3-dihydro-1H-benzo[d]imidazol-1-yl)piperidine-2,6-dione (A13.1)

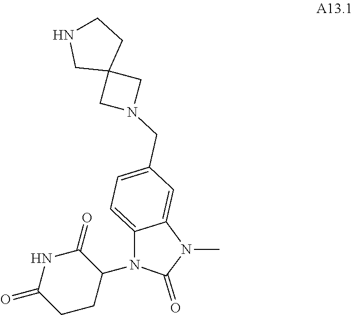 [SMILES: Cn1c(=O)n(C2CCC(=O)NC2=O)c2ccc(CN3CC4(CCNC4)C3)cc21]

A13.1. A13.1 was prepared analogously to A1.2 using A1.1 and tert-butyl 2,6-diazaspiro[3.4]octane-6-carboxylate as starting materials. ES/MS: m/z=384.2 [M+H]⁺.

### Preparation of 3-(5-(((1R,4R)-2,5-diazabicyclo[2.2.1]heptan-2-yl)methyl)-3-methyl-2-oxo-2,3-dihydro-1H-benzo[d]imidazol-1-yl)piperidine-2,6-dione (A14.1)

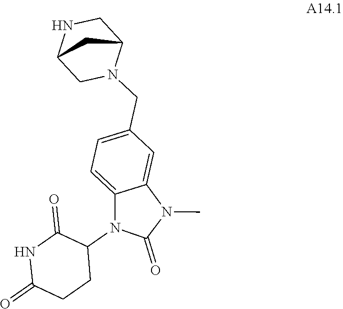 [SMILES: Cn1c(=O)n(C2CCC(=O)NC2=O)c2ccc(CN3C[C@H]4C[C@@H]3CN4)cc21]

A14.1. A14.1 was prepared analogously to A1.2 using A1.1 and tert-butyl (1R,4R)-2,5-diazabicyclo[2.2.1]heptane-2-carboxylate as starting materials. ES/MS: m/z=370.1 [M+H]⁺.

### Preparation of 3-(5-((3,8-diazabicyclo[3.2.1]octan-3-yl)methyl)-3-methyl-2-oxo-2,3-dihydro-1H-benzo[d]imidazol-1-yl)piperidine-2,6-dione (A15.1)

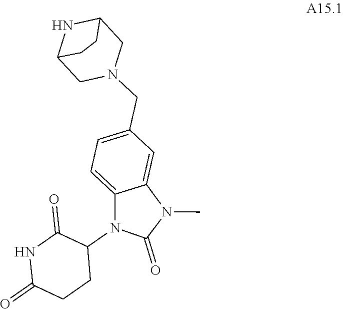 [SMILES: Cn1c(=O)n(C2CCC(=O)NC2=O)c2ccc(CN3CC4CCC(C3)N4)cc21]

A15.1. A15.1 was prepared analogously to A1.2 using A1.1 and tert-butyl 3,8-diazabicyclo[3.2.1]octane-8-carboxylate as starting materials. ES/MS: m/z=384.2 [M+H]⁺.

### Preparation of 3-(5-((3,6-diazabicyclo[3.1.1]heptan-3-yl)methyl)-3-methyl-2-oxo-2,3-dihydro-1H-benzo[d]imidazol-1-yl)piperidine-2,6-dione

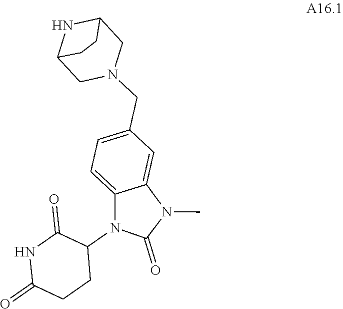 [SMILES: Cn1c(=O)n(C2CCC(=O)NC2=O)c2ccc(CN3CC4CCC(C3)N4)cc21]

A16.1. A16.1 was prepared analogously to A1.2 using A1.1 and tert-butyl 3,6-diazabicyclo[3.1.1]heptane-6-carboxylate as starting materials. (ESI): m/z=370.2 [M+H]⁺.

### Preparation of 3-(3-methyl-2-oxo-5-(4-(piperidin-4-yl)piperazin-1-yl)-2,3-dihydro-1H-benzo[d]imidazol-1-yl)piperidine-2,6-dione (A17.1)

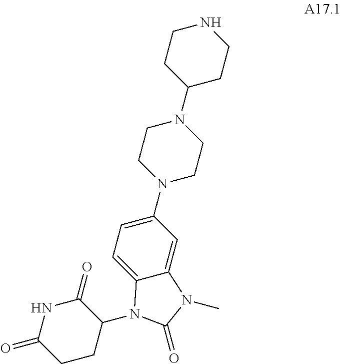 [SMILES: Cn1c(=O)n(C2CCC(=O)NC2=O)c2ccc(N3CCN(C4CCNCC4)CC3)cc21]

A17.1 was prepared analogously to A4.1 using tert-butyl 4-(piperazin-1-yl)piperidine-1-carboxylate. (ESI): m/z=427.2 [M+H]⁺.

### Preparation of 3-(5-(((S)-2,7-diazaspiro[4.4]nonan-2-yl)methyl)-3-methyl-2-oxo-2,3-dihydro-1H-benzo[d]imidazol-1-yl)piperidine-2,6-dione (A18.1)

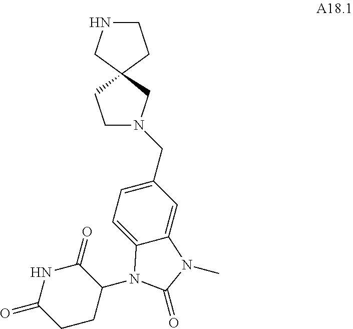 [SMILES: Cn1c(=O)n(C2CCC(=O)NC2=O)c2ccc(CN3CC[C@]4(CCNC4)C3)cc21]

A18.1. A18.1 was prepared analogously to A1.2 using A1.1 and tert-butyl (S)-2,7-diazaspiro[4.4]nonane-2-carboxylate as starting materials. ES/MS: m/z=398.2 [M+H]⁺.

### Preparation of 3-(5-(((3aR,6aS)-hexahydropyrrolo[3,4-c]pyrrol-2(1H)-yl)methyl)-3-methyl-2-oxo-2,3-dihydro-1H-benzo[d]imidazol-1-yl)piperidine-2,6-dione (A19.1)

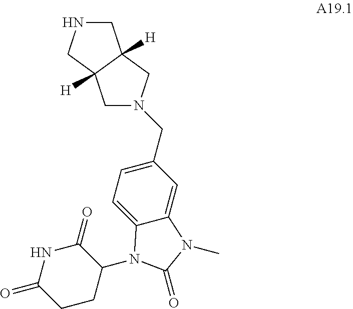 [SMILES: Cn1c(=O)n(C2CCC(=O)NC2=O)c2ccc(CN3C[C@H]4CNC[C@H]4C3)cc21]

A19.1. A19.1 was prepared analogously to A1.2 using A1.1 and tert-butyl (3aR,6aS)-hexahydropyrrolo[3,4-c]pyrrole-2(1H)-carboxylate as starting materials. ES/MS: m/z=383.2 [M+H]⁺.

### Preparation of 3-(5-(((R)-2,7-diazaspiro[4.4]nonan-2-yl)methyl)-3-methyl-2-oxo-2,3-dihydro-1H-benzo[d]imidazol-1-yl)piperidine-2,6-dione (A20.1)

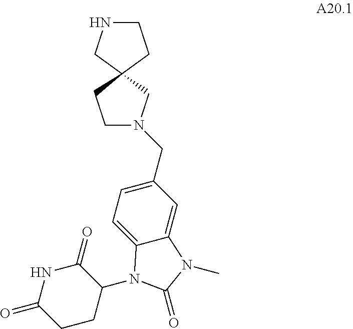 [SMILES: Cn1c(=O)n(C2CCC(=O)NC2=O)c2ccc(CN3CC[C@@]4(CCNC4)C3)cc21]

A20.1. A20.1 was prepared analogously to A1.2 using A1.1 and tert-butyl (R)-2,7-diazaspiro[4.4]nonane-2-carboxylate as starting materials. ES/MS: m/z=398.2 [M+H]⁺.

### Preparation of 3-(5-((2,6-diazaspiro[3.4]octan-6-yl)methyl)-3-methyl-2-oxo-2,3-dihydro-1H-benzo[d]imidazol-1-yl)piperidine-2,6-dione (A21.1)

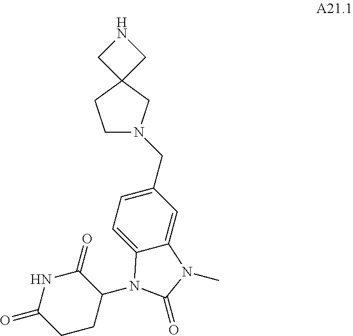 [SMILES: Cn1c(=O)n(C2CCC(=O)NC2=O)c2ccc(CN3CCC4(CNC4)C3)cc21]

A21.1. A21.1 was prepared analogously to A1.2 using A1.1 and tert-butyl 2,6-diazaspiro[3.4]octane-2-carboxylate as starting materials. (ESI): m/z=384.1 [M+H]⁺.

### Preparation of 1-(2,6-dioxopiperidin-3-yl)-4,6-difluoro-3-methyl-2-oxo-2,3-dihydro-1H-benzo[d]imidazole-5-carbaldehyde (A22.1)

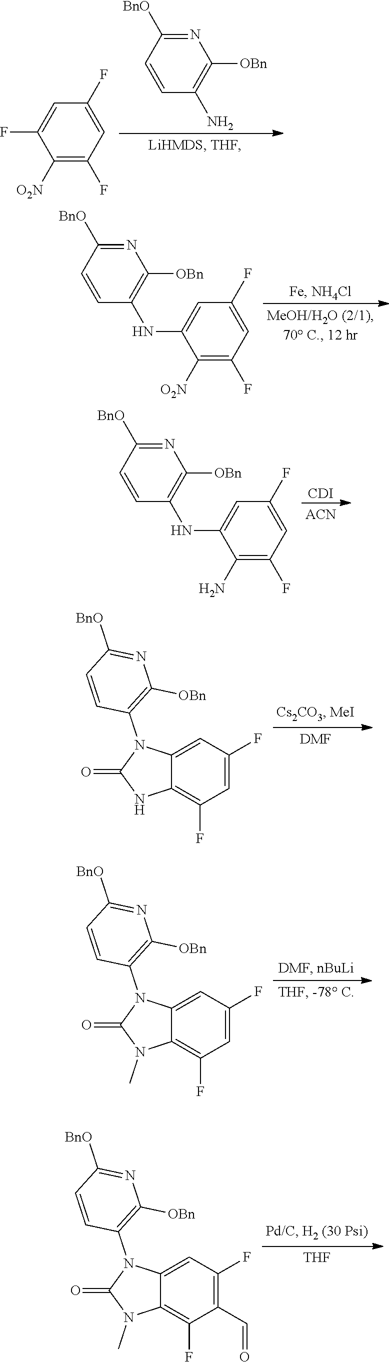

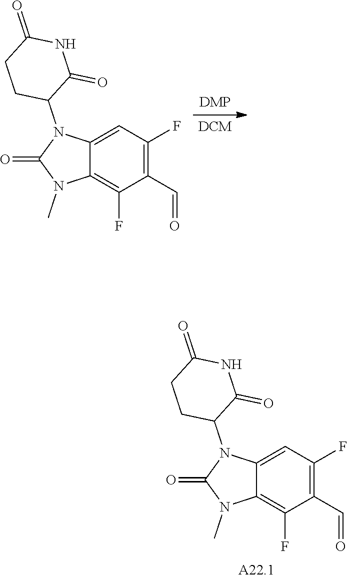

2,6-bis(benzyloxy)-N-(3,5-difluoro-2-nitrophenyl)pyridin-3-amine. A solution of 1,3,5-trifluoro-2-nitro-benzene (10.0 g, 0.0565 mol) and 2,6-dibenzyloxypyridin-3-amine (17.3 g, 0.0565 mol) in Tetrahydrofuran (100.0 mL) was cooled to −10° C. under N₂, Lithium bis(trimethylsilyl)amide (1.00 mol/L, 169 mL, 0.169 mol) was added dropwise to the mixture over 1 hr below 0° C. The mixture was stirred at 20° C. for 12 hr under N₂ atmosphere. The mixture was quenched by addition NH₄Cl 100 mL at 25° C., and extracted with EtOAc 150 mL (50 mL×3). The combined organic layers were washed with brine 100 mL (50 mL×2), dried over Na₂SO₄, filtered and concentrated under reduced pressure to give a residue. The residue was purified by flash silica gel chromatography to give the title compound. MS (ESI): m/z=464.2 [M+H]⁺

N1-(2,6-bis(benzyloxy)pyridin-3-yl)-3,5-difluorobenzene-1,2-diamine. To a solution of 2,6-dibenzyloxy-N-(3,5-difluoro-2-nitro-phenyl)pyridin-3-amine (20.0 g, 0.0432 mol) in MeOH (210 mL)/H₂O (105 mL) was added Fe (14.5 g, 0.259 mol) and ammonium chloride (5.77 g, 0.108 mol). The mixture was stirred at 70° C. for 12 hr. The mixture was filtered with EtOH (20 mL)/DCM (500 mL) and the filtrate was concentrated under reduced pressure to give a residue. Then the residue was diluted with H₂O 300 mL and extracted with Ethyl acetate 600 mL (200 mL×3). The combined organic layers were washed with brine 900 mL (300 mL×3), dried over Na₂SO₄, filtered and concentrated under reduced pressure to afford the title compound, which was used without further purification. MS (ESI): m/z=434.2 [M+H]⁺

1-(2,6-bis(benzyloxy)pyridin-3-yl)-4,6-difluoro-1,3-dihydro-2H-benzo[d]imidazol-2-one. To a solution of N1-(2,6-dibenzyloxy-3-pyridyl)-3,5-difluoro-benzene-1,2-diamine (18.0 g, 0.0415 mol) in ACN (360 mL) was added di(imidazol-1-yl)methanone (8.08 g, 0.0498 mol) at 20° C. The mixture was stirred at 25° C. for 12 hr. LC-MS showed Reactant 3 was consumed completely and desired compound was detected. The reaction mixture was diluted with H₂O 300 mL and extracted with Ethyl acetate 600 mL (200 mL×3). The combined organic layers were washed with brine 600 mL (200 mL×3), dried over Na₂SO₄, filtered and concentrated under reduced pressure to give a residue. The residue was triturated with MTBE (20 mL) at 25° C. for 1 hr. The residue was filtered, washed with MTBE (10 mL) and then the filter cake was dried under reduced pressure to give the title compound. MS (ESI): m/z=460.2 [M+H]⁺

1-(2,6-bis(benzyloxy)pyridin-3-yl)-4,6-difluoro-3-methyl-1,3-dihydro-2H-benzo[d]imidazol-2-one. To a solution of 3-(2,6-dibenzyloxy-3-pyridyl)-5,7-difluoro-1H-benzimidazol-2-one (14.0 g, 0.0305 mol) in Dimethylformamide (140.0 mL) was added cesium carbonate (19.9 g, 0.0609 mol) and iodomethane (8.65 g, 0.0609 mol). The mixture was stirred at 70° C. for 12 hr. LCMS sowed reactant 4 was consumed completely and desired mass was detected. The reaction mixture was diluted with H₂O 300 mL and extracted with Ethyl acetate 900 mL (300 mL×3). The combined organic layers were washed with brine 900 mL (300 mL×3), dried over Na₂SO₄, filtered and concentrated under reduced pressure to give a residue. The residue was triturated with MTBE (10 mL) at 25° C. for 1 hr. The residue was filtered, washed with MTBE (3 mL) and then the filter cake was dried under reduced pressure to give the title compound. ¹H NMR (400 MHz, CHLOROFORM-d) δ 7.50 (d, J=8.3 Hz, 1H), 7.38-7.25 (m, 5H), 7.19 (s, 3H), 7.18-7.14 (m, 2H), 6.59-6.51 (m, 1H), 6.45 (d, J=8.3 Hz, 1H), 6.17 (dd, J=1.8, 8.1 Hz, 1H), 5.40 (d, J=12.6 Hz, 1H), 5.29 (s, 2H), 5.20 (d, J=12.8 Hz, 1H), 3.56 (d, J=1.5 Hz, 3H). MS (ESI): m/z=474.2 [M+H]⁺

1-(2,6-bis(benzyloxy)pyridin-3-yl)-4,6-difluoro-3-methyl-2-oxo-2,3-dihydro-1H-benzo[d]imidazole-5-carbaldehyde. To a solution of 1-(2,6-dibenzyloxy-3-pyridyl)-4,6-difluoro-3-methyl-benzimidazol-2-one (4.00 g, 0.00845 mol) in Tetrahydrofuran (40.0 mL) was added dropwise N-Butyllithium solution 2.5 M in hexanes (2.50 mol/L, 4.39 mL, 0.0110 mol) at −78° C. under N₂. The mixture was stirred at −78° C. for 1 hr. Dimethylformamide (0.618 g, 0.00845 mol) in Tetrahydrofuran (2.0 mL) was added dropwise to the solution at −78° C. The mixture was stirred at −78° C. for 2 hr under N₂. The reaction mixture was quenched by addition of NH₄Cl 10 mL. Then extracted with ethyl acetate (15 mL×3). The combined organic layers were washed with brine (10 mL×3), dried over Na₂SO₄, filtered and concentrated under reduced pressure to give a residue. The residue was triturated with MTBE (10 mL). The residue was filtered, washed with MTBE (5 mL) and then the filter cake was dried under reduced pressure to give the title compound. ¹H NMR (400 MHz, CHLOROFORM-d) δ 10.32 (s, 1H), 7.59 (d, J=8.4 Hz, 1H), 7.47-7.36 (m, 5H), 7.33-7.29 (m, 3H), 7.28-7.24 (m, 2H), 6.56 (d, J=8.3 Hz, 1H), 6.32 (d, J=9.9 Hz, 1H), 5.50 (d, J=12.5 Hz, 1H), 5.40 (s, 2H), 5.32-5.26 (m, 1H), 3.68 (d, J=1.6 Hz, 3H). MS (ESI): m/z=502.2 [M+H]⁺

3-(4,6-difluoro-5-(hydroxymethyl)-3-methyl-2-oxo-2,3-dihydro-1H-benzo[d]imidazol-1-yl)piperidine-2,6-dione. To a bottle with Pd/C (10.0%, 2500 mg, 0.00235 mol) was added THF (100.00 mL) and 1-(2,6-dibenzyloxy-3-pyridyl)-4,6-difluoro-3-methyl-2-oxo-benzimidazole-5-carbaldehyde (2.50 g, 0.00499 mol) in THF (5.00 mL) under N₂. The mixture was stirred at 25° C. for 6 hr under H₂ at 30 psi. LC-MS showed Reactant 6 was consumed and desired compound mass was detected. The reaction mixture filtered, washed with 1000 mL THF and concentrated under reduced pressure to give a residue. The residue was triturated with MTBE (10 mL). The residue was filtered, washed with MTBE (5 mL) and then the filter cake was dried under reduced pressure to give the title compound. ¹H NMR (400 MHz, DMSO-d6) δ 11.14 (s, 1H), 7.09 (d, J=9.5 Hz, 1H), 5.42-5.33 (m, 1H), 5.21 (t, J=5.5 Hz, 1H), 4.53 (br d, J=5.1 Hz, 2H), 3.48 (d, J=1.4 Hz, 3H), 2.93-2.57 (m, 3H), 2.07-1.98 (m, 1H). MS (ESI): m/z=326.2[M+H]⁺

1-(2,6-dioxopiperidin-3-yl)-4,6-difluoro-3-methyl-2-oxo-2,3-dihydro-1H-benzo[d]imidazole-5-carbaldehyde (A22.2). To a solution of 3-[4,6-difluoro-5-(hydroxymethyl)-3-methyl-2-oxo-benzimidazol-1-yl]piperidine-2,6-dione (1.10 g, 0.00338 mol) in Dichloromethane (12.00 mL) was added Dess-Martin Periodinane (2.87 g, 0.00676 mol) at 0° C. The mixture was stirred at 20° C. for 2 hr. LC-MS showed the desired mass was detected. The residue was diluted with H₂O 10 mL and extracted with DCM (10 mL×3). The combined organic layers were washed with brine (10 mL×3), dried over Na₂SO₄, filtered and concentrated under reduced pressure to give a residue. The residue was purified by prep-HPLC (TFA condition: column: Phenomenex Luna C18 100×30 mm×5 um; mobile phase: [A: H₂O (0.1% TFA); B: ACN]; B %: 1.00%35.00%, 8.00 min; flow rate: 50.00 ml/min to give the title compound. ¹H NMR (400 MHz, DMSO-d6) δ 11.20 (s, 1H), 10.18 (s, 1H), 7.30 (d, J=11.0 Hz, 1H), 5.45 (dd, J=5.3, 12.7 Hz, 1H), 3.50 (s, 3H), 2.89-2.62 (m, 3H), 2.09-2.01 (m, 1H). MS (ESI): m/z=324.1 [M+H]⁺.

### Preparation of 1-(2,6-dioxopiperidin-3-yl)-4-fluoro-3-methyl-2-oxo-2,3-dihydro-1H-benzo[d]imidazole-5-carbaldehyde (A23.1)

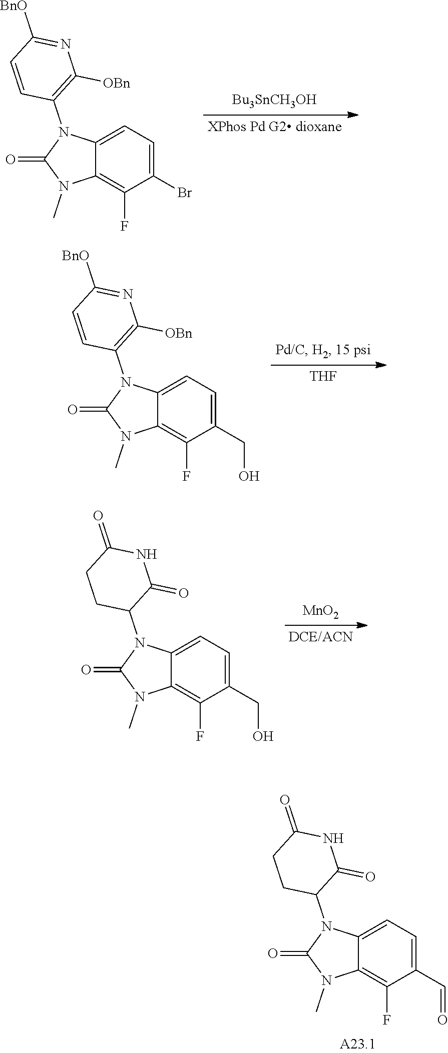

1-(2,6-bis(benzyloxy)pyridin-3-yl)-4-fluoro-5-(hydroxymethyl)-3-methyl-1,3-dihydro-2H-benzo[d]imidazol-2-one. A mixture of 5-bromo-1-(2,6-dibenzyloxy-3-pyridyl)-4-fluoro-3-methyl-benzimidazol-2-one (1.20 g, 0.00225 mol), tributylstannylmethanol (1.08 g, 0.00337 mol) and XPhos Pd G2 (0.265 g, 0.000337 mol) in 1,4-Dioxane (15.00 mL) was degassed and purged with N₂ for 3 times, the mixture was then stirred at 80° C. for 3 hr under N₂ atmosphere. The reaction mixture was concentrated under reduced pressure to give a residue. Then added 10 mL H₂O and extracted with ethyl acetate (20 mL×3). The combined organic layers were washed with brine (10 mL×3), dried over Na₂SO₄, filtered and concentrated under reduced pressure to give a residue. The residue was purified by flash silica gel chromatography to afford the title compound. ¹H NMR (400 MHz, CHLOROFORM-d) δ 7.59 (d, J=8.3 Hz, 1H), 7.46-7.32 (m, 5H), 7.29-7.21 (m, 5H), 7.02-6.95 (m, 1H), 6.58-6.44 (m, 2H), 5.50-5.41 (m, 1H), 5.36 (s, 2H), 5.32-5.23 (m, 1H), 4.78 (s, 2H), 3.65 (d, J=1.5 Hz, 3H). MS (ESI): m/z=486.3 [M+H]⁺

3-(4-fluoro-5-(hydroxymethyl)-3-methyl-2-oxo-2,3-dihydro-1H-benzo[d]imidazol-1-yl)piperidine-2,6-dione. To a bottle with Pd/C (10.0%, 0.25 g) was added THF (5.00 mL) and 1-(2,6-dibenzyloxy-3-pyridyl)-4-fluoro-5-(hydroxymethyl)-3-methyl-benzimidazol-2-one (0.50 g, 0.00103 mol) in THF (5.00 mL) under N₂. The mixture was stirred at 25° C. for 4 hr under H₂ at 15 psi. The reaction mixture filtered, washed with 300 mL THF and concentrated under reduced pressure to provide the title compound. MS (ESI): m/z=308.2 [M+H]⁺

1-(2,6-dioxopiperidin-3-yl)-4-fluoro-3-methyl-2-oxo-2,3-dihydro-1H-benzo[d]imidazole-5-carbaldehyde (A23.1). To a solution of 3-(4-fluoro-5-(hydroxymethyl)-3-methyl-2-oxo-2,3-dihydro-1H-benzo[d]imidazol-1-yl)piperidine-2,6-dione (0.350 g, 0.00114 mol) in DCE (5.00 mL) and ACN (5.0 mL) was added MANGANESE DIOXIDE (0.990 g, 0.0114 mol). The mixture was stirred at 25° C. for 12 hr. The mixture was filtered, washed with 200 mL THF and concentrated under reduced pressure to give a residue. The residue filtrate was purified by prep-HPLC (TFA condition, column: Phenomenex Luna C18 100*30 mm*5 um; mobile phase: [A: H₂O (0.1% TFA); B: ACN]; B %: 10.00%-40.00%, 8.00 min; flow rate: 50.00 ml/min) to give the title compound. ¹H NMR (400 MHz, DMSO-d6) δ 11.19 (s, 1H), 10.14 (s, 1H), 7.62-7.51 (m, 1H), 7.21 (d, J=8.4 Hz, 1H), 5.53-5.43 (m, 1H), 3.54 (d, J=1.3 Hz, 3H), 2.95-2.82 (m, 1H), 2.78-2.68 (m, 1H), 2.68-2.60 (m, 1H), 2.11-2.03 (m, 1H). MS (ESI): m/z=306.2 [M+H]⁺

### Preparation of perfluorophenyl 5-((diethoxyphosphoryl)difluoromethyl)benzo[b]thiophene-2-carboxylate (B1.1)

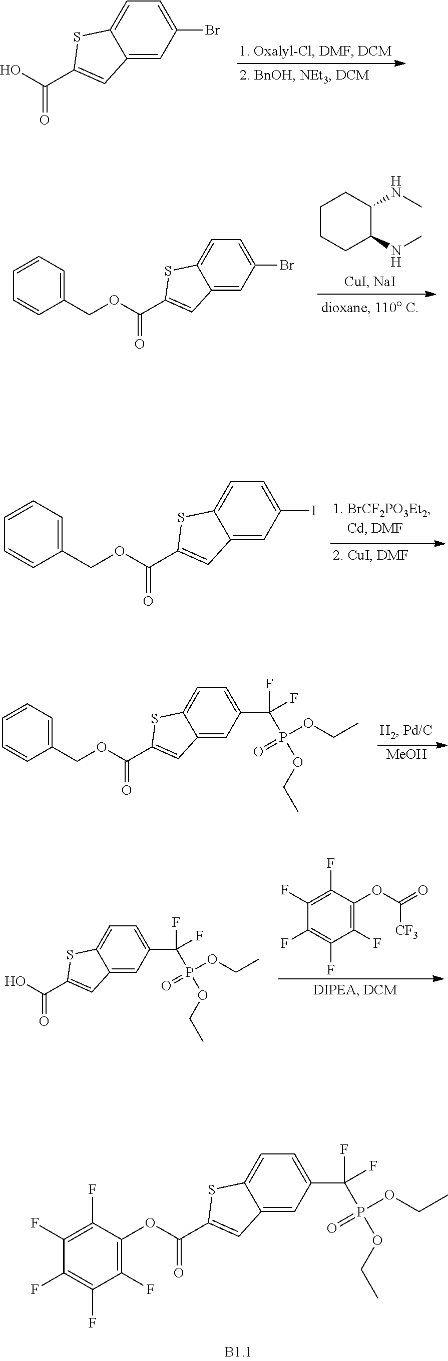

benzyl 5-bromobenzo[b]thiophene-2-carboxylate. To a stirred solution of 5-bromo-1-benzothiophene-2-carboxylic acid (100 g, 389 mmol) in DCM (1000 mL) was added (COCl)₂ (99.4 mL, 1167 mmol, 3.00 equiv) dropwise at 0° C. To the above mixture was added DMF (3.01 mL, 38.9 mmol, 0.10 equiv) dropwise at 0° C. The resulting mixture was stirred at room temperature for additional 2 h and was concentrated under reduced pressure affording 5-bromobenzo[b]thiophene-2-carbonyl chloride which was used directly without further purification. To a stirred solution of benzyl alcohol (46.27 g, 427.880 mmol, 1.10 equiv) and TEA (59.04 g, 583.431 mmol, 1.5 equiv) in DCM (1000 mL) was added 5-bromobenzo[b]thiophene-2-carbonyl chloride dropwise at room temperature. The resulting mixture was stirred at room temperature for 2 h. The resulting mixture was concentrated under reduced pressure. The resulting mixture was extracted with EtOAc (3×500 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (10:1) to afford benzyl 5-bromo-1-benzothiophene-2-carboxylate. 1H NMR (300 MHz, DMSO-d6) δ 8.30-8.26 (m, 1H), 8.23-8.20 (m, 1H), 8.09-8.03 (m, 1H), 7.68 (dd, J=8.7, 2.0 Hz, 1H), 7.54-7.32 (m, 5H), 5.40 (s, 2H).

benzyl 5-iodobenzo[b]thiophene-2-carboxylate. To a stirred solution of benzyl 5-bromo-1-benzothiophene-2-carboxylate (80 g, 230 mmol, 1 equiv) and (1S,2S)-1-N,2-N-dimethylcyclohexane-1,2-diamine (6.55 g, 46.0 mmol, 0.20 equiv) in 1,4-dioxane (800 mL) was added CuI (4.39 g, 23.1 mmol, 0.10 equiv) and NaI (69.07 g, 460.8 mmol, 2 equiv) at room temperature. The resulting mixture was stirred at 110° C. for 72 h. The mixture was allowed to cool down to room temperature. The resulting mixture was filtered, the filter cake was washed with DCM (3×500 mL). The filtrate was concentrated under reduced pressure. The residue was purified by silica gel column chromatography, eluted with PE/EA (10:1) to afford benzyl 5-iodo-1-benzothiophene-2-carboxylate. ¹H NMR (300 MHz, Chloroform-d) δ 8.24 (d, J=1.6 Hz, 1H), 8.01-7.99 (m, 1H), 7.72 (dd, J=8.6, 1.6 Hz, 1H), 7.64-7.59 (m, 1H), 7.52-7.34 (m, 5H), 5.42 (s, 2H).

benzyl 5-((diethoxyphosphoryl)difluoromethyl)benzo[b]thiophene-2-carboxylate. To a stirred solution of cadmium (70 g, 614 mmol, 3.5 equiv) in DMF (300 mL) was added diethyl (bromodifluoromethyl)phosphonate (95 g, 355 mmol, 2 equiv) dropwise at 0° C. under a nitrogen atmosphere. The resulting mixture was stirred at room temperature for 30 min under a nitrogen atmosphere. The resulting mixture was filtered, the filter cake was washed with DMF (10 mL). To a stirred solution of benzyl 5-iodo-1-benzothiophene-2-carboxylate (70.00 g, 177.561 mmol, 1 equiv) and CuI (67.63 g, 355.122 mmol, 2 equiv) in DMF (400 mL) was added the above solution dropwise at room temperature. The resulting mixture was stirred at room temperature for 16 h. The resulting mixture was extracted with EtOAc (3×500 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (3:1) to afford benzyl 5-[(diethoxyphosphoryl)difluoromethyl]-1-benzothiophene-2-carboxylate. ¹H NMR (400 MHz, Chloroform-d) δ 8.18-8.12 (m, 2H), 7.96 (d, J=8.6 Hz, 1H), 7.74-7.68 (m, 1H), 7.52-7.34 (m, 5H), 5.42 (s, 2H), 4.32-4.11 (m, 4H), 1.34 (t, J=7.1 Hz, 6H).

5-((diethoxyphosphoryl)difluoromethyl)benzo[b]thiophene-2-carboxylic acid. To a stirred solution of benzyl 5-[(diethoxyphosphoryl)difluoromethyl]-1-benzothiophene-2-carboxylate (41.00 g, 90.225 mmol, 1 equiv) in MeOH (450 mL) was added Pd/C (0.96 g, 9.022 mmol, 0.1 equiv) at room temperature under nitrogen atmosphere. The resulting mixture was stirred at room temperature for 16 h under a hydrogen atmosphere. The reaction was monitored by LCMS. The resulting mixture was filtered, the filter cake was washed with MeOH (3×200 mL). The filtrate was concentrated under reduced pressure affording 5-[(diethoxyphosphoryl)difluoromethyl]-1-benzothiophene-2-carboxylic acid. ¹H NMR (400 MHz, DMSO-d₆) δ 13.66 (s, 1H), 8.31-8.21 (m, 3H), 7.68-7.61 (m, 1H), 4.24-4.05 (m, 4H), 1.23 (t, J=7.0 Hz, 6H).

perfluorophenyl 5-((diethoxyphosphoryl)difluoromethyl)benzo[b]thiophene-2-carboxylate (B1.1). To a stirred solution of 5-[(diethoxyphosphoryl)difluoromethyl]-1-benzothiophene-2-carboxylic acid (25 g, 68.625 mmol, 1 equiv) and 2,3,4,5,6-pentafluorophenyl 2,2,2-trifluoroacetate (23.08 g, 82.350 mmol, 1.2 equiv) in DCM (500 mL) was added DIPEA (17.75 g, 137.250 mmol, 2 equiv) at room temperature under air atmosphere. The resulting mixture was stirred for 1 h at room temperature under air. The resulting mixture was concentrated under reduced pressure. The residue was purified by reversed-phase flash chromatography with the following conditions: column, C18 gel; mobile phase, MeCN in Water (0.1% FA), 40% to 70% gradient. Desired fractions were collected at and concentrated under reduced pressure to afford 2,3,4,5,6-pentafluorophenyl 5-[(diethoxyphosphoryl)difluoromethyl]-1-benzothiophene-2-carboxylate. 1H NMR (400 MHz, Chloroform-d) δ 8.41 (s, 1H), 8.26 (s, 1H), 8.04 (d, J=8.7 Hz, 1H), 7.81 (d, J=8.7 Hz, 1H), 4.39-4.20 (m, 4H), 1.36 (t, J=7.1 Hz, 6H). ES/MS: m/z=531.0 [M+H]⁺.

### Preparation of 5-((diethoxyphosphoryl)fluoromethyl)benzo[b]thiophene-2-carboxylic acid (B2.1)

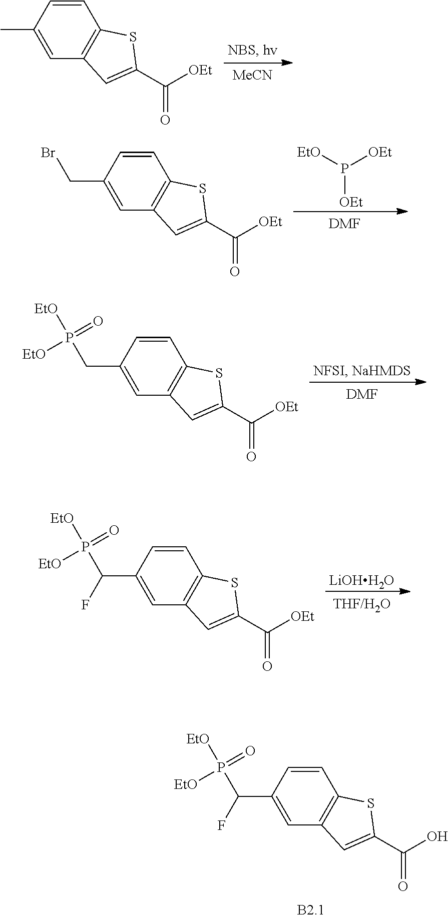

ethyl 5-(bromomethyl)benzo[b]thiophene-2-carboxylate. Solution 1: ethyl 5-methylbenzo[b]thiophene-2-carboxylate (100 g, 453.9 mmol, 1 eq) and NBS (96.9 g, 544.7 mmol, 1.2 eq) in MeCN (1000 mL). The volume of flow reactor 1 {⅛″ PEA coil} was {55 mL}. The residence time of flow reactor 1 was {15 min}. Set the bath at {40° C.} for flow reactor 1, hv=365 nm 200 W×2. The flow rate of Pump 1 was adjusted to 3.67 mL/min for solution 1. The mixture was collected with a bottle. The Pump 1 was started and the reaction mixture was collected after running 9 hr. The reaction mixture was concentrated under reduced pressure to give a residue. The reaction mixture was diluted with aq. Na₂SO₃ 1000 mL at 25° C., and extracted with EtOAc (500 mL×3). The combined organic layers were washed with brine (500 mL×2), dried over Na₂SO₄, filtered and concentrated under reduced pressure to give ethyl 5-(bromomethyl)benzo[b]thiophene-2-carboxylate which was used without further purification. ¹H NMR (400 MHz, chloroform-d) δ 8.03 (s, 1H), 7.90-7.83 (m, 2H), 7.50 (dd, J=1.6, 8.4 Hz, 1H), 4.64 (s, 2H), 4.46-4.37 (m, 2H), 1.43 (t, J=7.1 Hz, 3H). MS (ESI): m/z=299.1 [M+H]⁺.

ethyl 5-((diethoxyphosphoryl)methyl)benzo[b]thiophene-2-carboxylate. To a solution of ethyl 5-(bromomethyl)benzo[b]thiophene-2-carboxylate (10 g, 26.7 mmol, 1 eq) in DMF (100 mL) stirred at 20° C. was added triethyl phosphite (4.8 g, 29.4 mmol, 5.0 mL, 1.1 eq). The mixture was heated to 100° C. and stirred for 3 hrs. The reaction mixture was diluted H₂O 40 mL at 25° C., and extracted with EtOAc (30 mL×3). The combined organic layers were washed with brine (30 mL×2), dried over Na₂SO₄, filtered and concentrated under reduced pressure to give a residue. The residue was purified by flash silica gel chromatography (0 to 100% EtOAc/petroleum ether gradient) to give the title compound. ¹H NMR (400 MHz, CDCl₃) δ 8.04-8.00 (m, 1H), 7.83-7.79 (m, 2H), 7.44-7.39 (m, 1H), 4.41 (q, J=7.1 Hz, 2H), 4.10-3.98 (m, 4H), 1.42 (t, J=7.1 Hz, 3H), 1.25 (t, J=7.1 Hz, 6H). MS (ESI): m/z=357.2 [M+H]⁺.

ethyl 5-((diethoxyphosphoryl)fluoromethyl)benzo[b]thiophene-2-carboxylate. A mixture of ethyl 5-((diethoxyphosphoryl)methyl)benzo[b]thiophene-2-carboxylate(1 g, 2.8 mmol) in THF (20 mL) was degassed and purged with N₂ for 3 times and cooled −70° C. NaHMDS (1 M, 3.3 mL) was added to the solution. The mixture was stirred at −70° C. for 30 min under N₂ atmosphere. Then NFSI (884.8 mg, 2.8 mmol) in THF (4 mL) was added to the solution. The mixture was stirred at −70° C. for 1 h under N₂ atmosphere. The reaction mixture was quenched by addition sat. NH₄Cl aq 100 mL at 25° C., and extracted with EtOAc (20 mL×3). The combined organic layers were washed with brine (10 mL×2), dried over Na₂SO₄, filtered and concentrated under reduced pressure to give a residue. The residue was purified by flash silica gel chromatography (0 to 100% EtOAc/petroleum ether gradient) to give ethyl 5-((diethoxyphosphoryl)fluoromethyl)benzo[b]thiophene-2-carboxylate. 1H NMR (400 MHz, CHLOROFORM-d) δ 8.07 (s, 1H), 8.00 (s, 1H), 7.91 (d, J=8.5 Hz, 1H), 7.59 (br d, J=8.5 Hz, 1H), 5.89-5.74 (m, 1H), 4.43 (q, J=7.1 Hz, 2H), 4.23-4.02 (m, 4H), 1.43 (t, J=7.1 Hz, 3H), 1.29 (dt, J=4.5, 7.0 Hz, 6H). MS (ESI): m/z=375.2 [M+H]⁺.

rac-5-((diethoxyphosphoryl)fluoromethyl)benzo[b]thiophene-2-carboxylic acid (B2.1). To a solution of ethyl 5-((diethoxyphosphoryl)fluoromethyl)benzo[b]thiophene-2-carboxylate (1.2 g, 3.3 mmol) in EtOH (6.8 mL) and H₂O (2.7 mL) was added LiOH monohydrate (307.5 mg, 7.3 mmol). The mixture was stirred at 20° C. for 1 hr. The residue was diluted with H₂O 5 mL and added HCl (1 M) to adjust the pH to 3 and extracted with EtOAc (10 mL×3). The combined organic layers were washed with brine (10 mL×2), dried over Na₂SO₄, filtered, and concentrated under reduced pressure to give the title compound. MS (ESI): m/z=346.9 [M+H]⁺.

### Chiral SFC Separation of B2.1 to B3.1 and B3.2:

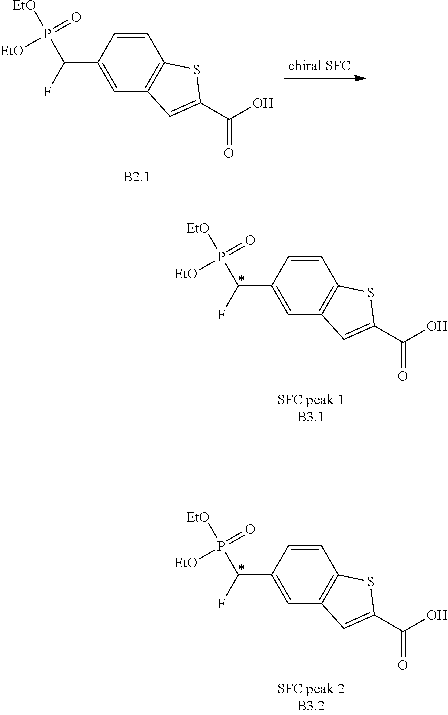 [SMILES: CCOP(=O)(OCC)C(F)c1ccc2sc(C(=O)O)cc2c1 ; CCOP(=O)(OCC)C(F)c1ccc2sc(C(=O)O)cc2c1 ; CCOP(=O)(OCC)C(F)c1ccc2sc(C(=O)O)cc2c1]

5-diethoxyphosphoryl(fluoro)methyl]benzothiophene-2-carboxylic acid (B2.1, 1.2 g) was purified by prep-HPLC (TFA) and was further separated by prep-SFC (column: Daicel ChiralPak IG (250*30 mm, 10 um); mobile phase: [A: CO₂; B: EtOH]; B %: 30.00%-30.00%, 6.30 min) to isolate the first eluting SFC peak 5-((diethoxyphosphoryl)fluoromethyl)benzo[b]thiophene-2-carboxylic acid (B3.1, 330 mg, RT=2.25 min) and the second eluting SFC peak 5-((diethoxyphosphoryl)fluoromethyl)benzo[b]thiophene-2-carboxylic acid (B3.2, 340 mg, RT=2.51 min).

First eluting SFC peak, B3.1: ¹H NMR (400 MHz, DMSO-d₆) δ 13.82-13.42 (m, 1H), 8.19 (s, 1H), 8.16-8.07 (m, 2H), 7.57 (br d, J=8.4 Hz, 1H), 6.36-6.17 (m, 1H), 4.13-3.93 (m, 4H), 1.25-1.12 (m, 6H).

Second eluting SFC peak, B3.2: ¹H NMR (400 MHz, DMSO-d₆) δ 13.77-13.39 (m, 1H), 8.19 (s, 1H), 8.15-8.05 (m, 2H), 7.57 (br d, J=8.3 Hz, 1H), 6.36-6.17 (m, 1H), 4.13-3.93 (m, 4H), 1.28-1.13 (m, 6H).

### Preparation of perfluorophenyl 5-((diethoxyphosphoryl)fluoromethyl)benzo[b]thiophene-2-carboxylate (B3.3)

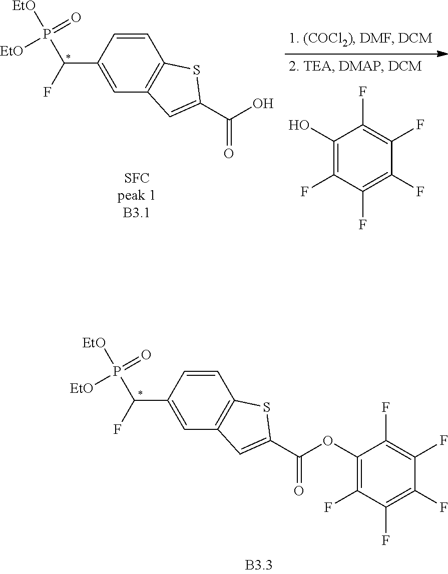 [SMILES: CCOP(=O)(OCC)C(F)c1ccc2sc(C(=O)O)cc2c1 ; Oc1c(F)c(F)c(F)c(F)c1F ; CCOP(=O)(OCC)C(F)c1ccc2sc(C(=O)Oc3c(F)c(F)c(F)c(F)c3F)cc2c1]

To a solution of B3.1 (0.330 g, 0.953 mmol) in DCM (6.0 mL) was added oxalyl chloride (0.122 mL, 1.43 mmol) and two drops Dimethylformamide at 0° C. The mixture was stirred at 0° C. for 0.5 hr. The reaction mixture was concentrated under reduced pressure to remove solvent. The residue was dissolved in DCM (6.0 mL) and followed by addition of 4-dimethylaminopyridine (58.2 mg, 0.4 mmol), triethylamine (0.397 mL, 2.8 mmol) and 2,3,4,5,6-pentafluorophenol (228 mg, 1.2 mmol) in DCM (0.5 mL) at 0° C. under N₂. The mixture was stirred at 20° C. for 2 hr. The mixture was concentrated under reduced pressure to remove solvent. The reaction mixture was diluted with H₂O 20 mL and extracted with ethyl acetate (20 mL×3). The combined organic layers were washed with brine (10 mL×3), dried over Na₂SO₄, filtered, and concentrated under reduced pressure to give a residue. The residue was purified by prep-HPLC (TFA condition, column: Phenomenex Luna C18 100*30 mm*5 um; mobile phase: [A: H₂O (0.1% TFA); B: ACN]; B %: 45.00%-75.00%, 8.00 min) to give B3.3. ¹H NMR (400 MHz, DMSO-d₆) δ 8.76 (s, 1H), 8.30-8.22 (m, 2H), 7.70 (br d, J=8.5 Hz, 1H), 6.42-6.26 (m, 1H), 4.14-3.95 (m, 4H), 1.26-1.13 (m, 6H). MS (ESI): m/z=513.1 [M+H]⁺.

### Preparation of perfluorophenyl 5-((diethoxyphosphoryl)fluoromethyl)benzo[b]thiophene-2-carboxylate (B3.4)

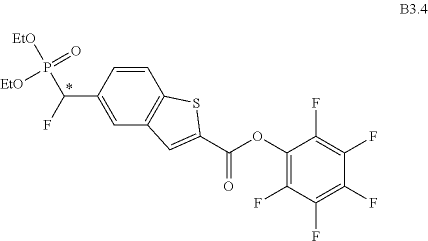 [SMILES: CCOP(=O)(OCC)C(F)c1ccc2sc(C(=O)Oc3c(F)c(F)c(F)c(F)c3F)cc2c1]

B3.4 was prepared analogously to B3.3 using B3.2 (SFC peak 2) instead of B3.1. ¹H NMR (400 MHz, DMSO-d₆) δ 8.75 (s, 1H), 8.29-8.20 (m, 2H), 7.70 (br d, J=8.6 Hz, 1H), 6.44-6.26 (m, 1H), 4.15-3.94 (m, 4H), 1.26-1.13 (m, 6H). MS (ESI): m/z=513.1 [M+H]⁺.

### Preparation of perfluorophenyl 5-(fluoro((((S)-1-oxo-1-propoxypropan-2-yl)amino)(phenoxy)phosphoryl)methyl)benzo[b]thiophene-2-carboxylate (B4.1)

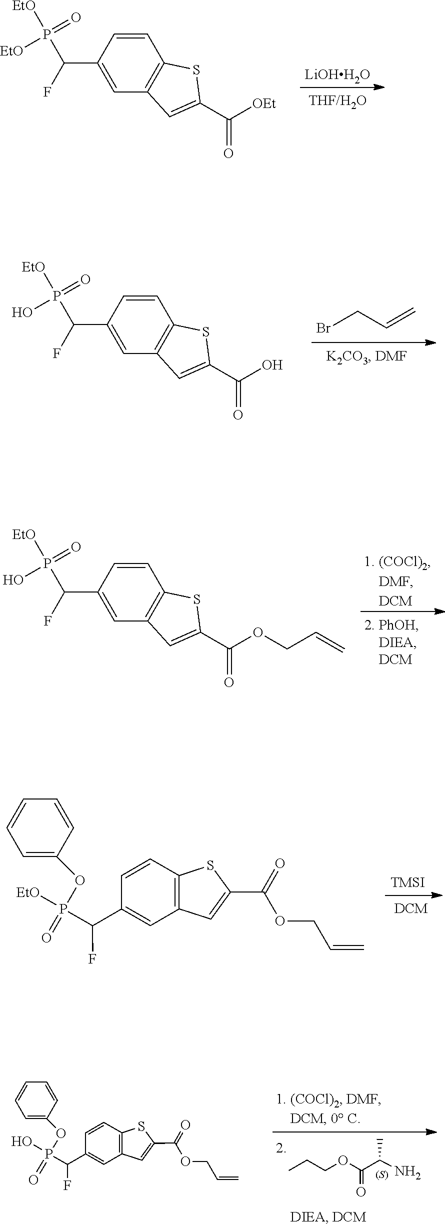

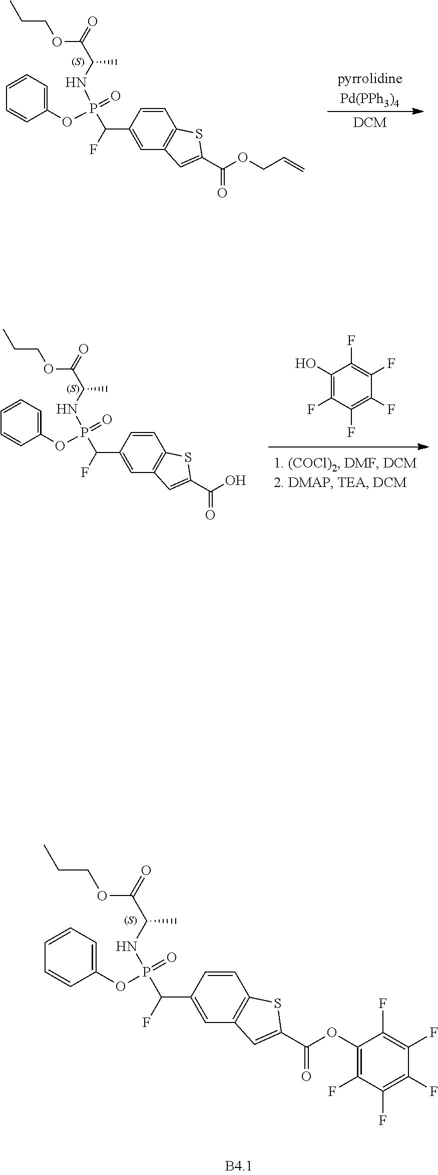

5-((ethoxy(hydroxy)phosphoryl)fluoromethyl)benzo[b]thiophene-2-carboxylic acid. To a solution of ethyl 5-[diethoxyphosphoryl(fluoro)methyl]benzothiophene-2-carboxylate (5.0 g, 13.4 mmol) in THF (28.0 mL)/H₂O (11.0 mL) was added lithium hydroxide monohydrate (1.1 g, 6.7 mmol). The mixture was stirred at 20° C. for 3 hrs. The reaction mixture was adjusted pH to 3-4 by 1M HCl and extracted with ethyl acetate (10 mL×3). The combined organic layers were dried over Na₂SO₄, filtered and concentrated under reduced pressure to give 5-((ethoxy(hydroxy)phosphoryl)fluoromethyl)benzo[b]thiophene-2-carboxylic acid and was used into the next step without further purification. MS (ESI): m/z=318.8 [M+H]⁺.

allyl 5-((ethoxy(hydroxy)phosphoryl)fluoromethyl)benzo[b]thiophene-2-carboxylate. To a solution of 5-[[ethoxy(hydroxy)phosphoryl]-fluoro-methyl]benzothiophene-2-carboxylic acid (4.5 g, 14.1 mmol) in dimethylformamide (50 mL) was added potassium carbonate (5.8 g, 42.4 mmol) and 3-bromoprop-1-ene (2.5 g, 21.2 mmol). The mixture was stirred at 20° C. for 12 hrs. The reaction mixture was diluted with H₂O (50 mL) and extracted with ethyl acetate (50 mL×3). The combined organic layers were washed with brine (50 mL×3), dried over Na₂SO₄, filtered and concentrated under reduced pressure to give allyl 5-((ethoxy(hydroxy)phosphoryl)fluoromethyl)benzo[b]thiophene-2-carboxylate which was used in the next step without further purification. MS (ESI): m/z=358.9 [M+H]⁺.

allyl 5-((ethoxy(phenoxy)phosphoryl)fluoromethyl)benzo[b]thiophene-2-carboxylate. To a solution of allyl 5-((ethoxy(hydroxy)phosphoryl)fluoromethyl)benzo[b]thiophene-2-carboxylate (2.0 g, 5.5 mmol) in DCM (30.0 mL) at 0° C. was added 0.05 mL dimethylformamide and oxalyl chloride (2.1 mL, 25.1 mmol). The mixture was stirred at 0° C. for 1 hr. The reaction mixture was concentrated under reduced pressure to remove solvent affording allyl 5-((chloro(ethoxy)phosphoryl)fluoromethyl)benzo[b]thiophene-2-carboxylate and was used into the next step without further purification.

To a solution of allyl 5-[[chloro(ethoxy)phosphoryl]-fluoro-methyl]benzothiophene-2-carboxylate (76.0%, 2.1 g, 4.2 mmol) in DCM (30.0 mL) was added phenol (0.79 g, 8.4 mmol) in dichloromethane (10.0 mL) at 0° C. under N₂ atmosphere. Then N,N-diisopropylethylamine (3.5 mL, 21.2 mmol) was added dropwise at 0° C. under N₂ atmosphere. The mixture was stirred at 20° C. for 14 hrs under N₂ atmosphere. The reaction mixture was then diluted with H₂O 20 mL and extracted with DCM (20 mL×3). The combined organic layers were dried over Na₂SO₄, filtered, and concentrated under reduced pressure to give a residue. The residue was purified by flash silica gel chromatography (20 g Flash Silica Column, Eluent of 0-60% Ethyl acetate/Petroleum ether gradient @100 mL/min) to give allyl 5-((ethoxy(phenoxy)phosphoryl)fluoromethyl)benzo[b]thiophene-2-carboxylate. ¹H NMR (400 MHz, chloroform-d) δ 8.09 (s, 1H), 8.03 (s, 1H), 7.91 (d, J=8.5 Hz, 1H), 7.62 (br d, J=8.5 Hz, 1H), 7.37-7.28 (m, 2H), 7.22-7.16 (m, 2H), 6.14-6.02 (m, 1H), 6.02-5.87 (m, 1H), 5.50-5.42 (m, 1H), 5.37-5.31 (m, 1H), 4.87 (d, J=5.6 Hz, 2H), 4.28-4.12 (m, 2H), 1.31-1.18 (m, 4H). MS (ESI): m/z=435.0 [M+H]⁺.

allyl 5-(fluoro(hydroxy(phenoxy)phosphoryl)methyl)benzo[b]thiophene-2-carboxylate. To a mixture of allyl 5-[[ethoxy(phenoxy)phosphoryl]-fluoro-methyl]benzothiophene-2-carboxylate (1800 mg, 3.9 mmol) in DCM (30.00 mL) was added trimethylsilyl iodide (1.4 mL, 9.9 mmol) at 0° C. under N₂ atmosphere. The mixture stirred at 0° C. for 1 h under N₂ atmosphere. The mixture was concentrated under reduced pressure to remove solvent affording allyl 5-(fluoro(hydroxy(phenoxy)phosphoryl)methyl)benzo[b]thiophene-2-carboxylate which was used in the next step without further purification. MS (ESI): m/z=406.9 [M+H]⁺.

allyl 5-(fluoro((((S)-1-oxo-1-propoxypropan-2-yl)amino)(phenoxy)phosphoryl)methyl)benzo[b]thiophene-2-carboxylate. To a solution of allyl 5-(fluoro(hydroxy(phenoxy)phosphoryl)methyl)benzo[b]thiophene-2-carboxylate (95.7%, 2.0 g, 4.7 mmol) in DCM (20.0 mL) at 0° C. was added 0.05 mL dimethylformamide and oxalyl chloride (1.2 mL, 14.1 mmol). The mixture was stirred at 0° C. for 1 h and then the mixture was concentrated under reduced pressure. The reside was dissolved in in DCM (20.0 mL) and was added N,N-diisopropylethylamine (3.9 mL, 23.5 mmol) and propyl (2S)-2-aminopropanoate hydrochloride (0.9 g, 5.6 mmol) in DCM (5.0 mL) at 0° C. The mixture was stirred at 20° C. for 1 hr. The mixture was then diluted with H₂O 20 mL and extracted with DCM. The combined organic layers were dried over Na₂SO₄, filtered, and concentrated under reduced pressure to give a residue. The residue was purified by flash silica gel chromatography (12 g Flash Silica Column, Eluent of 0-60% Ethyl acetate/Petroleum ether gradient @100 mL/min) to give allyl 5-(fluoro((((S)-1-oxo-1-propoxypropan-2-yl)amino)(phenoxy)phosphoryl)methyl)benzo[b]thiophene-2-carboxylate. ¹H NMR (400 MHz, chloroform-d) δ 8.15-8.06 (m, 1H), 8.05-7.97 (m, 1H), 7.93-7.85 (m, 1H), 7.69-7.57 (m, 1H), 7.39-7.33 (m, 1H), 7.27-7.17 (m, 2H), 7.15-7.05 (m, 2H), 6.15-5.85 (m, 2H), 5.53-5.30 (m, 2H), 4.87 (d, J=5.6 Hz, 2H), 4.09-3.94 (m, 2H), 3.68-3.49 (m, 1H), 1.68-1.59 (m, 2H), 1.32-1.26 (m, 3H), 0.96-0.82 (m, 3H). MS (ESI): m/z=520.1 [M+H]⁺.

5-(fluoro((((S)-1-oxo-1-propoxypropan-2-yl)amino)(phenoxy)phosphoryl)methyl)benzo[b]thiophene-2-carboxylic acid. To a solution of allyl 5-(fluoro((((S)-1-oxo-1-propoxypropan-2-yl)amino)(phenoxy)phosphoryl)methyl)benzo[b]thiophene-2-carboxylate (1.3 g, 2.5 mmol) in dichloromethane (20.0 mL) was added tetrakis(triphenylphosphine)palladium(0) (0.28 g, 0.25 mmol) and pyrrolidine (0.5 mL, 6.2 mmol) at 0° C. under N₂ atmosphere. The mixture was stirred at 0° C. for 2 hrs. The reaction mixture was concentrated under reduced pressure to remove solvent to afford 5-(fluoro((((S)-1-oxo-1-propoxypropan-2-yl)amino)(phenoxy)phosphoryl)methyl)benzo[b]thiophene-2-carboxylic acid was used in the next step without further purification. MS (ESI): m/z=480.0 [M+H]⁺.

perfluorophenyl 5-(fluoro((((S)-1-oxo-1-propoxypropan-2-yl)amino)(phenoxy)phosphoryl)methyl)benzo[b]thiophene-2-carboxylate (B4.1). To a solution of 5-(fluoro((((S)-1-oxo-1-propoxypropan-2-yl)amino)(phenoxy)phosphoryl)methyl)benzo[b]thiophene-2-carboxylic acid (48.0%, 2.0 g, 2.0 mmol) in DCM (20.0 mL) was added oxalyl chloride (0.34 mL, 4.0 mmol) and 0.05 mL dimethylformamide at 0° C. The mixture was stirred at 0° C. for 0.5 hr. The mixture was concentrated under reduced pressure to remove solvent. The residue was dissolved in DCM (20.00 mL) and followed by addition of 4-dimethylaminopyridine (0.048 g, 0.4 mmol), triethylamine (0.8 mL, 6.0 mmol) and 2,3,4,5,6-pentafluorophenol (0.55 g, 3.0 mmol) in DCM (10 mL) at 0° C. The mixture was stirred at 20° C. for 2 hrs. The mixture was diluted with H₂O 20 mL and extracted with DCM (20 mL×3). The combined organic layers were dried over Na₂SO₄, filtered, and concentrated under reduced pressure to give a residue. The residue was purified by flash silica gel chromatography (20 g Flash Silica Column, Eluent of 0-30% Ethyl acetate/Petroleum ether gradient @100 mL/min) and was then further purified by prep-HPLC (TFA condition, column: Phenomenex Luna C18 100*30 mm*5 um; mobile phase: [A: H₂O (0.1% TFA); B: ACN]; B %: 65.00%-85.00%, 8.00 min) to give perfluorophenyl 5-(fluoro((((S)-1-oxo-1-propoxypropan-2-yl)amino)(phenoxy)phosphoryl)methyl)benzo[b]thiophene-2-carboxylate. ¹H NMR (400 MHz, chloroform-d) δ 8.34 (s, 1H), 8.14-8.06 (m, 1H), 7.99-7.90 (m, 1H), 7.76-7.65 (m, 1H), 7.41-7.34 (m, 1H), 7.32-7.27 (m, 1H), 7.26-7.23 (m, 1H), 7.23-7.12 (m, 1H), 7.08 (br t, J=7.6 Hz, 1H), 6.13-5.88 (m, 1H), 4.20-3.95 (m, 2H), 3.84-3.54 (m, 1H), 1.71-1.47 (m, 2H), 1.36-1.10 (m, 3H), 0.97-0.81 (m, 3H). MS (ESI): m/z=646.2 [M+H]⁺.

### Preparation of allyl 5-((diethoxyphosphoryl)fluoromethyl)benzo[b]thiophene-2-carboxylate (B5.1) and allyl 5-((diethoxyphosphoryl)fluoromethyl)benzo[b]thiophene-2-carboxylate (B5.2)

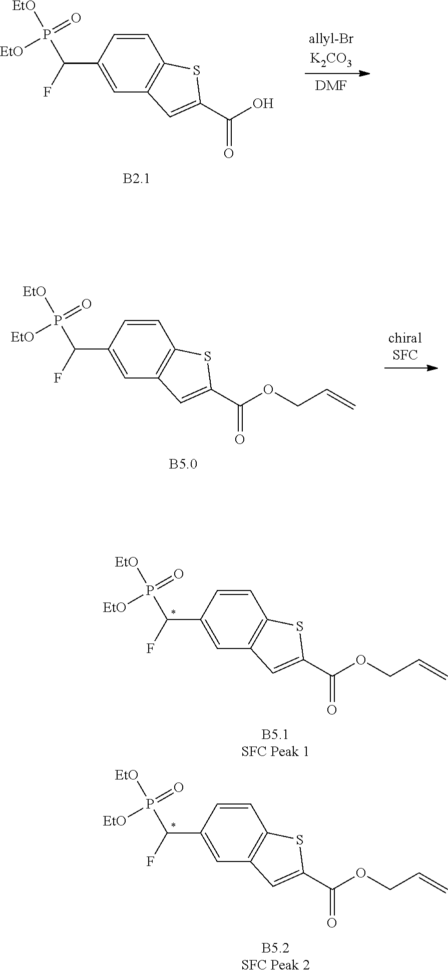

allyl 5-((diethoxyphosphoryl)fluoromethyl)benzo[b]thiophene-2-carboxylate (B5.0). To a stirred mixture of 5-((diethoxyphosphoryl)fluoromethyl)benzo[b]thiophene-2-carboxylic acid (200 g, 0.57 mol) in 1000 mL of N,N-dimethylformamide was added K₂CO₃ (159.53 g, 1.16 mmol, 2.00 equiv) and allyl bromide (75.24 g, 0.63 mmol) in portions at 0° C. under nitrogen atmosphere. The resulting mixture was stirred for overnight at room temperature under nitrogen atmosphere. The reaction was quenched with 2000 mL of water at 0° C. The resulting mixture was extracted with 3×2000 mL of dichloromethane. The organic layers were combined and washed with 2×1000 mL of brine and dried over anhydrous Na₂SO₄ and then filtered. The filtrate was then concentrated under reduced pressure to afford allyl 5-((diethoxyphosphoryl)fluoromethyl)benzo[b]thiophene-2-carboxylate. LCMS (ESI) [M+H]+: 386.08.

allyl 5-((diethoxyphosphoryl)fluoromethyl)benzo[b]thiophene-2-carboxylate (B5.1) and allyl 5-((diethoxyphosphoryl)fluoromethyl)benzo[b]thiophene-2-carboxylate (B5.2). Allyl 5-((diethoxyphosphoryl)fluoromethyl)benzo[b]thiophene-2-carboxylate (145.0 g) was separated by chiral SFC (conditions: instrument=Waters 350 Preparative SFC, column=REGIS(S,S) WHELK-01, 250×50 mm I.D. 10 μm, mobile phase=A for CO₂ and B for MeOH (Neu), gradient=B 30%, flow rate=220 g/min, back pressure=100 bar, column temperature=35° C., wavelength=220 nm, cycle-time=3.3 min) to afford allyl 5-((diethoxyphosphoryl)fluoromethyl)benzo[b]thiophene-2-carboxylate (B5.1) (first eluting peak on Prep-SFC) and allyl 5-((diethoxyphosphoryl)fluoromethyl)benzo[b]thiophene-2-carboxylate (B5.2) (second eluting peak on Prep-SFC). The second eluting peak was purified again by reverse-phase chromatography with the following condition: C18 column, 30% acetonitrile in water (0.5% HCOOH)) as light-yellow oil).

First eluting SFC Peak, B5.1: 1H NMR (400 MHz, Chloroform-d) δ 8.09 (s, 1H), 7.99 (d, J=2.5 Hz, 1H), 7.90 (d, J=8.5 Hz, 1H), 7.59 (dd, J=8.6, 1.8 Hz, 1H), 6.04 (dd, J=16.2, 10.8, 5.6 Hz, 1H), 5.81 (dd, J=44.6, 7.7 Hz, 1H), 5.44 (dd, J=17.1, 1.6 Hz, 1H), 5.33 (dd, J=10.5, 1.5 Hz, 1H), 4.86 (dt, J=5.6, 1.5 Hz, 2H), 4.23-4.00 (m, 4H), 1.28 (td, J=7.1, 4.0 Hz, 6H). LCMS [M+H]+: 387.05.

Second eluting SFC Peak, B5.2: ¹H NMR (300 MHz, Chloroform-d) δ 8.11 (s, 1H), 8.01 (d, J=2.5 Hz, 1H), 7.92 (d, J=8.5 Hz, 1H), 7.59 (dd, J=8.6, 1.8 Hz, 1H), 6.04 (dd, J=16.2, 10.8, 5.6 Hz, 1H), 5.81 (dd, J=44.6, 7.7 Hz, 1H), 5.44 (dd, J=17.1, 1.6 Hz, 1H), 5.33 (dd, J=10.5, 1.5 Hz, 1H), 4.86 (dt, J=5.6, 1.5 Hz, 2H), 4.23-4.00 (m, 4H), 1.28 (td, J=7.1, 4.0 Hz, 6H). LCMS [M+H]+: 387.05.

### Preparation of perfluorophenyl 5-(fluoro((((S)-1-oxo-1-propoxypropan-2-yl)amino)(phenoxy)phosphoryl)methyl)benzo[b]thiophene-2-carboxylate (SFC peak 1, B5.7) and perfluorophenyl 5-(fluoro((((S)-1-oxo-1-propoxypropan-2-yl)amino)(phenoxy)phosphoryl)methyl)benzo[b]thiophene-2-carboxylate (SFC peak 2, B5.8)

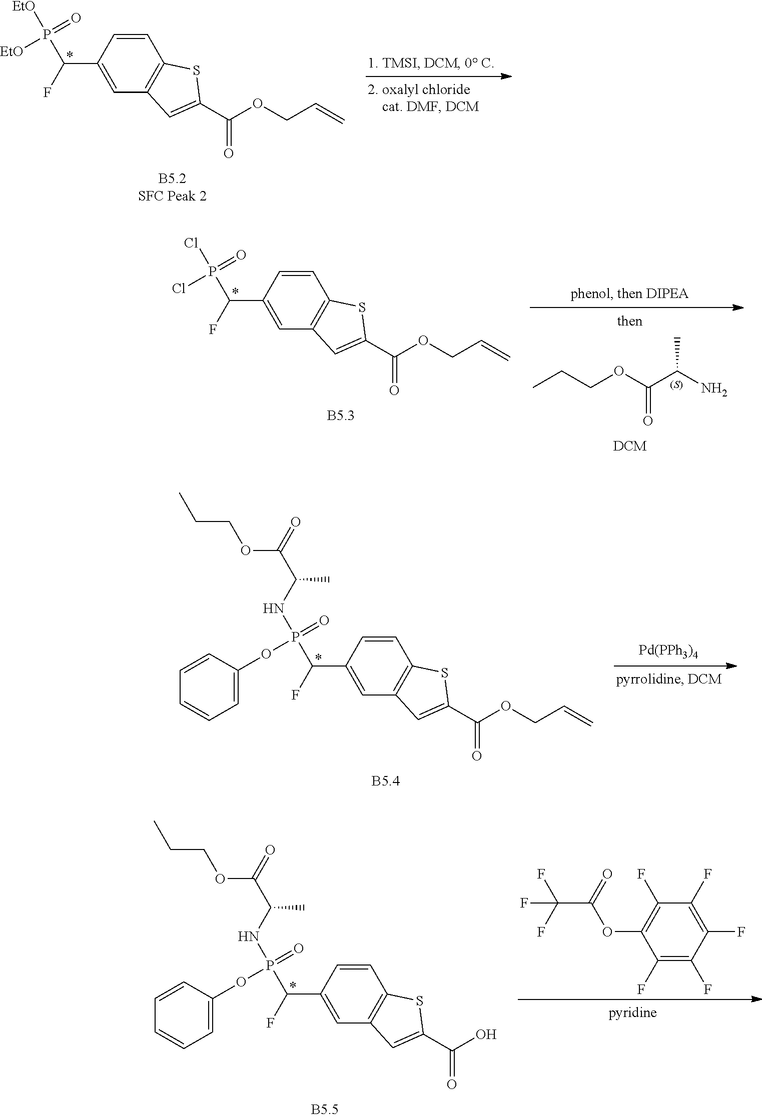

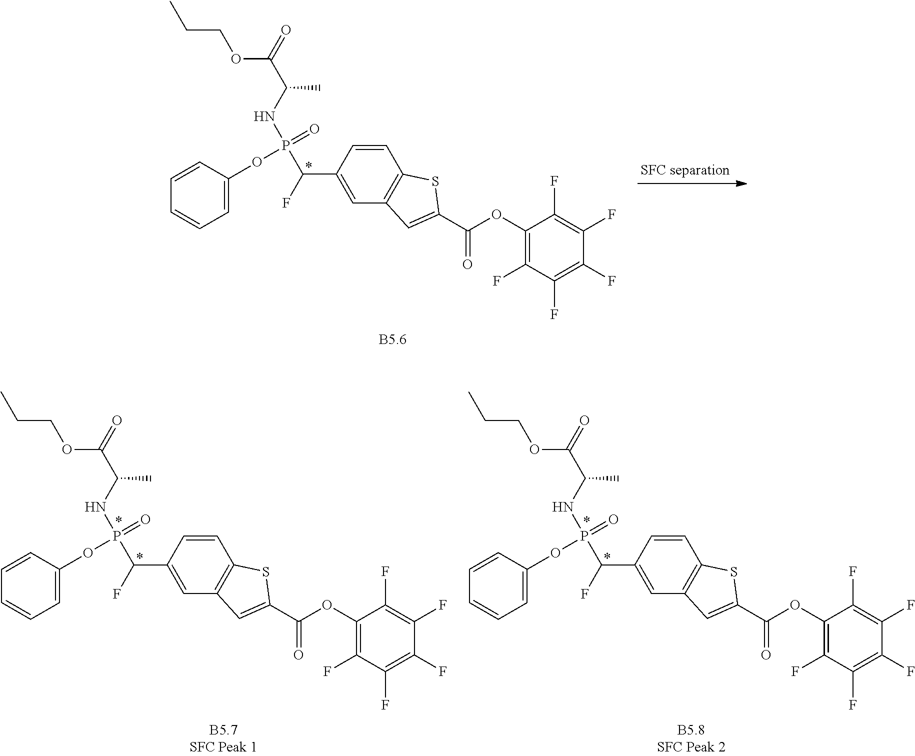

allyl 5-((dichlorophosphoryl)fluoromethyl)benzo[b]thiophene-2-carboxylate (B5.3). To a stirred solution of B5.2 (10.00 g, 25.88 mmol) in 200 mL of dichloromethane was added iodotrimethylsilane (10.36 g, 51.76 mmol) dropwise at 0° C. under nitrogen atmosphere. The resulting mixture was stirred for 2 h at room temperature under nitrogen atmosphere. The resulting mixture was concentrated under reduced pressure. The residue was purified by silica gel column chromatography, eluted with 20% methanol in dichloromethane to afford ((2-((allyloxy)carbonyl)benzo[b]thiophen-5-yl)fluoromethyl)phosphonic acid.

To a stirred solution of ((2-((allyloxy)carbonyl)benzo[b]thiophen-5-yl)fluoromethyl)phosphonic acid (10.00 g, 30.27 mmol) and N, N-dimethylformamide (0.22 g, 3.02 mmol) in 130 mL of dichloromethane was added oxalyl chloride (11.53 g, 90.83 mmol) dropwise at 0° C. The resulting mixture was stirred at 0° C. for 30 minutes and then warmed to 40° C. for 2 h. The resulting mixture was concentrated under reduced pressure to afford allyl 5-((dichlorophosphoryl)fluoromethyl)benzo[b]thiophene-2-carboxylate (B5.3) and was used in the next step directly without further purification. LCMS [M+H]⁺: 366.94.

allyl 5-(fluoro((((S)-1-oxo-1-propoxypropan-2-yl)amino)(phenoxy)phosphoryl)methyl)benzo[b]thiophene-2-carboxylate (B5.4). To a solution of B5.3 (8.00 g, 21.78 mmol) in 100 mL of dichloromethane was added phenol (1.64 g, 17.43 mmol) in 100 mL of dichloromethane in 10 minutes at 0° C., then to the resulting mixture was added a solution of N, N-diisopropylethylamine (8.45 g, 65.36 mmol) in 100 mL of dichloromethane dropwise over 20 minutes and the mixture was stirred at room temperature for 10 min, then to the resulting mixture was added a solution of propyl (2S)-2-aminopropanoate (2.86 g, 21.78 mmol) in 100 mL of dichloromethane, and the mixture was stirred for 1 h at room temperature. The resulting mixture was concentrated under reduced pressure. The residue was purified by silica gel column chromatography, eluted with 50% ethyl acetate in petroleum ether to afford allyl 5-(fluoro((((S)-1-oxo-1-propoxypropan-2-yl)amino)(phenoxy)phosphoryl)methyl)benzo[b]thiophene-2-carboxylate B5.4. LCMS [M+H]⁺: 520.13.

5-(fluoro((((S)-1-oxo-1-propoxypropan-2-yl)amino)(phenoxy)phosphoryl)methyl)benzo[b]thiophene-2-carboxylic acid (B5.5). To a stirred solution of B5.4 (5.00 g, 9.62 mmol) in 40 mL of dichloromethane was added pyrrolidine (0.55 g, 7.69 mmol) dropwise at 0° C. under nitrogen atmosphere, then to the resulting mixture was added tetrakis(triphenylphosphine)palladium (1.67 g, 1.445 mmol) dropwise and stirred at 0° C. for 10 minutes and warmed to room temperature for 10 minutes. The resulting mixture was then concentrated under reduced pressure. The residue was purified by silica gel column chromatography, eluted with 50% methanol in dichloromethane to afford B5.5. LCMS [M+H]⁺: 480.10.

perfluorophenyl 5-(fluoro((((S)-1-oxo-1-propoxypropan-2-yl)amino)(phenoxy)phosphoryl)methyl)benzo[b]thiophene-2-carboxylate (B5.6). To a stirred solution of B5.5 (11.00 g, 22.94 mmol) in 80 mL of pyridine was added 2,3,4,5,6-pentafluorophenyl 2,2,2-trifluoroacetate (19.28 g, 68.82 mmol) dropwise at 0° C. The resulting mixture was stirred for 1 h at room temperature. The resulting mixture was concentrated under reduced pressure and purified by silica gel column chromatography, eluted with 50% ethyl acetate in petroleum ether to afford B5.6. LCMS [M+H]⁺: 646.08.

SFC peak 1 perfluorophenyl 5-(fluoro((((S)-1-oxo-1-propoxypropan-2-yl)amino)(phenoxy)phosphoryl)methyl)benzo[b]thiophene-2-carboxylate (B5.7) and SFC peak 2 perfluorophenyl 5-((R)-fluoro((R or perfluorophenyl 5-(fluoro((((S)-1-oxo-1-propoxypropan-2-yl)amino)(phenoxy)phosphoryl)methyl)benzo[b]thiophene-2-carboxylate (B5.8). B5.6 (8.00 g) was purified by Prep-SFC with the following conditions (Column: CHIRAL ART Cellulose-SB, 4.6*50 mm, 3 μm; Mobile Phase B: EtOH; Gradient: isocratic % B) to obtain perfluorophenyl 5-(fluoro((((S)-1-oxo-1-propoxypropan-2-yl)amino)(phenoxy)phosphoryl)methyl)benzo[b]thiophene-2-carboxylate B5.7 (first eluting peak on Prep-SFC) and perfluorophenyl 5-(fluoro((((S)-1-oxo-1-propoxypropan-2-yl)amino)(phenoxy)phosphoryl)methyl)benzo[b]thiophene-2-carboxylate B5.8 (second eluting peak on Prep-SFC). LCMS [M+H]+: 646.10.

First eluting SFC peak, B5.7: ¹H NMR (400 MHz, Methanol-d₄) δ 8.50 (s, 1H), 8.23 (s, 1H), 8.10 (s, 1H), 7.80 (s, 1H), 7.22-7.12 (m, 2H), 7.17-7.19 (m, 3H), 6.21 (d, J=6.5 Hz, 1H), 3.93 (s, 1H), 3.90-3.78 (m, 2H), 1.51 (m, 2H), 1.21 (m, 3H), 0.86 (m, 3H).

Second eluting SFC peak, B5.8: ¹H NMR (400 MHz, Methanol-d₄) δ 8.50 (s, 1H), 8.23 (s, 1H), 8.11 (d, J=8.6 Hz, 1H), 7.78 (dt, J=8.6, 1.8 Hz, 1H), 7.38-7.30 (m, 2H), 7.25-7.14 (m, 3H), 6.17 (d, J=7.0 Hz, 1H), 3.98 (m, 2H), 3.88 (s, 1H), 1.57 (m, 2H), 1.23 (m, 3H), 0.89 (m, 3H).

### Preparation of dipropyl 2,2′-(((fluoro(2-((perfluorophenoxy)carbonyl)benzo[b]thiophen-5-yl)methyl)phosphoryl)bis(azanediyl))(2S,2′S)-dipropionate (B6.4)

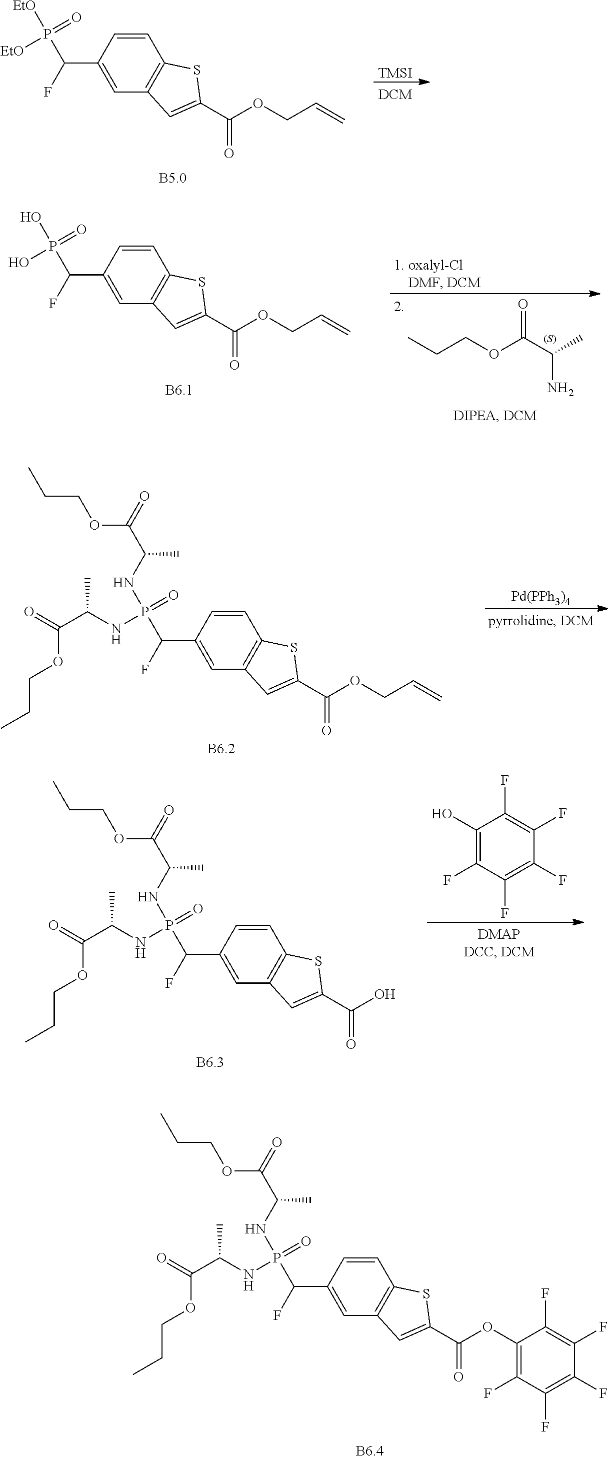

((2-((allyloxy)carbonyl)benzo[b]thiophen-5-yl)fluoromethyl)phosphonic acid (B6.1). To a mixture of allyl 5-[diethoxyphosphoryl(fluoro)methyl]benzothiophene-2-carboxylate (B5.0, 500 mg, 1.2 mmol) in DCM (5.0 mL) was added trimethylsilyl iodide (0.448 mL, 3.0 mmol) at 0° C. under N₂ atmosphere. The mixture was stirred at 0° C. for 30 min. The reaction mixture was concentrated under reduced pressure to give B6.1 which was used in the next step without further purification.

dipropyl 2,2′-((((2-((allyloxy)carbonyl)benzo[b]thiophen-5-yl)fluoromethyl)phosphoryl)bis(azanediyl))(2S,2'S)-dipropionate (B6.2). To a solution of B6.1 (0.4 g, 1.0 mmol) in DCM (5.0 mL) at 0° C. was added dimethylformamide (0.1 mL, 5.4 mmol) and oxalyl chloride (0.279 mL, 3.2 mmol). The mixture was stirred at 0° C. for 0.5 hr. The reaction mixture was concentrated under reduced pressure to remove solvent. The reside was dissolved in DCM (20.0 mL) and was added N,N-diisopropylethylamine (1.8 mL, 10.9 mmol) and propyl (2S)-2-aminopropanoate;hydrochloride (0.4 g, 2.4 mmol) in DCM (10.0 mL) at 0° C. The mixture was stirred at 20° C. for 2 hr. The reaction mixture was then diluted with H₂O 10 mL and extracted with DCM (20 mL×3). The combined organic layers were dried over Na₂SO₄, filtered, and concentrated under reduced pressure. The residue was purified by flash silica gel chromatography (4 g Flash Silica Column, Eluent of 0-60% Ethyl acetate/Petroleum ether gradient @80 mL/min) to give B6.2. MS (ESI): m/z=557.3 [M+H]⁺.

5-((bis(((S)-1-oxo-1-propoxypropan-2-yl)amino)phosphoryl)fluoromethyl)benzo[b]thiophene-2-carboxylic acid (B6.3). To a solution of B6.2 (0.3 g, 0.5 mmol) in dichloromethane (5.0 mL) was added Tetrakis(triphenylphosphine)palladium(0) (0.062 g, 5.39e-5 mol) and pyrrolidine (0.1 mL, 1.3 mmol) at 0° C. under N₂ atmosphere. The mixture was stirred at 20° C. for 1 hr. The reaction mixture was concentrated under reduced pressure to give crude B6.3 and was used into the next step without further purification. MS (ESI): m/z=517.2 [M+H]⁺.

dipropyl 2,2′-(((fluoro(2-((perfluorophenoxy)carbonyl)benzo[b]thiophen-5-yl)methyl)phosphoryl)bis(azanediyl))(2S,2'S)-dipropionate (B6.4). To a solution of B6.3 (0.4 g, 0.38 mmol), 2,3,4,5,6-pentafluorophenol (0.09 g, 0.5 mmol) and 4-Dimethylaminopyridine (0.00416 g, 3.41e-5 mol) in DCM (5.0 mL) was added N,N′-Dicyclohexylcarbodiimide (0.09 g, 0.47 mmol) in DCM (1.00 mL) at 0° C. The mixture was stirred at 20° C. for 3 hr under N₂. The reaction mixture was diluted with H₂O 10 mL and extracted with DCM (10 mL×3). The combined organic layers were dried over Na₂SO₄, filtered, and concentrated under reduced pressure to give a residue. The residue filtrate was purified by prep-HPLC (TFA condition, column: Phenomenex Luna C18 100*30 mm*5 um; mobile phase: [A: H₂O (0.1% TFA); B: ACN]; B %: 50.00%-80.00%, 8.00 min) to give B6.4. ¹H NMR (400 MHz, chloroform-d) δ 8.34 (d, J=2.6 Hz, 1H), 8.08 (br s, 1H), 7.99-7.91 (m, 1H), 7.70 (br t, J=8.6 Hz, 1H), 5.99-5.82 (m, 1H), 4.21-4.00 (m, 4H), 4.00-3.74 (m, 2H), 3.60-3.42 (m, 2H), 1.75-1.59 (m, 3H), 1.54-1.42 (m, 4H), 1.31-1.20 (m, 3H), 1.02-0.79 (m, 6H). MS (ESI): m/z=683.2 [M+H]⁺.

### Preparation of (difluoro(2-((perfluorophenoxy)carbonyl)benzo[b]thiophen-5-yl)methyl)phosphonic acid (B7.1)

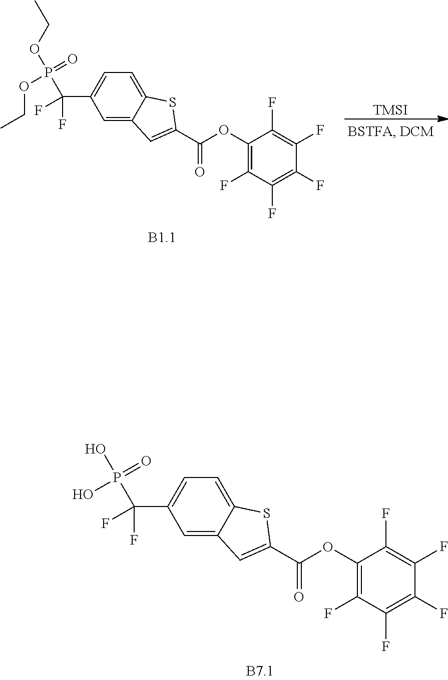 [SMILES: "TMSI" ; CCOP(=O)(OCC)C(F)(F)c1ccc2sc(C(=O)Oc3c(F)c(F)c(F)c(F)c3F)cc2c1 ; O=C(Oc1c(F)c(F)c(F)c(F)c1F)c1cc2cc(C(F)(F)P(=O)(O)O)ccc2s1]

B1.1 (350 mg, 0.66 mmol) was charged to a vial, dissolved in DCM (10.4 mL), and cooled to ° C. N,O-Bis(trimethylsilyl)trifluoroacetamide (1.58 mL, 5.94 mmol) was added followed by Iodotrimethylsilane (2.64 mL of a 1M solution in DCM, 2.64 mmol) and the mixture was stirred for 20 minutes at 0° C. then concentrated under reduced pressure. Purification by reverse-phase HPLC (MeCN/water, TFA) afforded the title compound (B7.1). ES/MS: m/z=472.9 [M−H]−0.1H NMR (400 MHz, DMSO-d6) δ 8.81 (s, 1H), 8.40-8.27 (m, 2H), 7.76 (d, J=8.7 Hz, 1H).

### Preparation of ethyl (R or S)-5-((diethoxyphosphoryl)fluoromethyl)-4-fluorobenzo[b]thiophene-2-carboxylate (B8.2) and (R or S)-5-((diethoxyphosphoryl)fluoromethyl)-4-fluorobenzo[b]thiophene-2-carboxylate (B8.3)

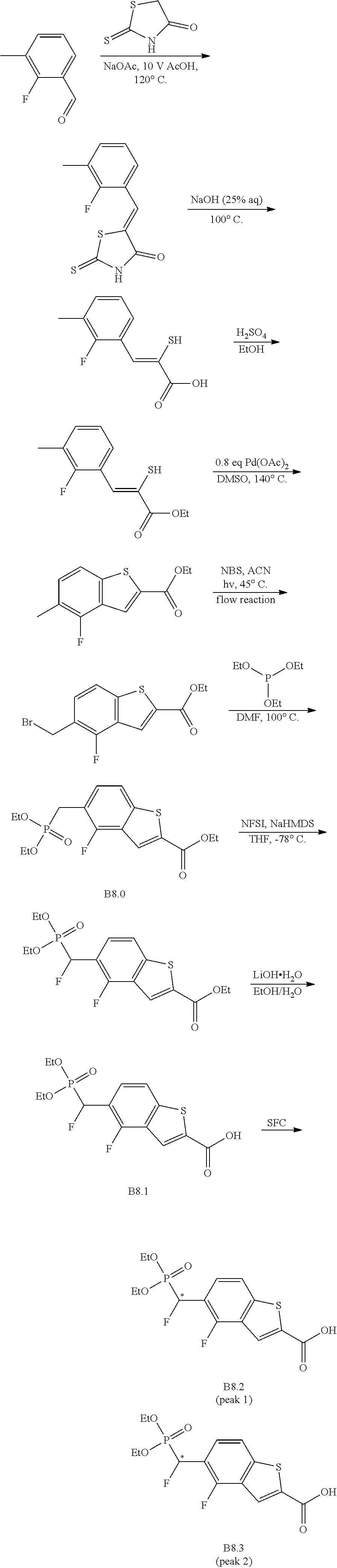

5-(2-fluoro-3-methylbenzylidene)-2-thioxothiazolidin-4-one. A mixture of 2-fluoro-3-methyl-benzaldehyde (50.0 g, 0.362 mol), 2-thioxothiazolidin-4-one (48.2 g, 0.362 mol) and sodium acetate (35.6 g, 0.434 mol) in acetic acid (500.0 mL) was stirred at 120° C. for 12 hr. The mixture was filtered, washed with 500 mL H₂O and the solid was dried under reduced pressure to give the title compound as a mixture of isomers. ¹H NMR (400 MHz, DMSO-d₆) δ 14.29-13.38 (m, 1H), 7.57 (s, 1H), 7.42 (br t, J=6.1 Hz, 1H), 7.33-7.15 (m, 2H), 2.26 (br s, 3H).

3-(2-fluoro-3-methylphenyl)-2-mercaptoacrylic acid. A solution of 5-(2-fluoro-3-methylbenzylidene)-2-thioxothiazolidin-4-one (150 g, 0.592 mol) in sodium hydroxide (750 mL, 25% w/v, aq) was stirred at 100° C. for 1 hr. The mixture was washed with 500 mL DCM. Then water layer was adjusted to pH 3-4 by addition of 6 M HCl, then extracted with ethyl acetate (1000 mL×3). The combined organic layers were dried over Na₂SO₄, filtered and concentrated under reduced pressure to give the title compound. MS (ESI): m/z=211.1 [M-1]

ethyl 3-(2-fluoro-3-methylphenyl)-2-mercaptoacrylate. To a solution of 3-(2-fluoro-3-methylphenyl)-2-mercaptoacrylic acid (50.0 g, 0.236 mol) in EtOH (500 mL) was added sulfuric acid (60.3 mL, 1.13 mol) at 0° C. The mixture was stirred at 90° C. for 12 hr (2 batches). The mixture was concentrated under reduced pressure to remove solvent. The mixture was diluted with H₂O 500 mL at 0° C. and extracted with ethyl acetate (500 mL×3). The combined organic layers were washed with brine (200 mL×3), dried over Na₂SO₄, filtered and concentrated under reduced pressure to give a residue. The residue was purified by flash silica gel chromatography to afford the title compound as a mixture of isomers. ¹H NMR (400 MHz, DMSO-d₆) δ 7.27-7.00 (m, 3H), 5.55-4.88 (m, 1H), 4.24-3.98 (m, 2H), 2.24-2.16 (m, 3H), 1.27-0.98 (m, 3H). MS (ESI): m/z=239.1 [M-1]

ethyl 4-fluoro-5-methylbenzo[b]thiophene-2-carboxylate. A mixture of ethyl 3-(2-fluoro-3-methylphenyl)-2-mercaptoacrylate (21.0 g, 0.0874 mol) and palladium(II) acetate (15.7 g, 0.0699 mol) in DMSO (520.0 mL) was degassed and purged with N₂ for 3 times, the mixture was then stirred at 140° C. for 12 hr under N₂ atmosphere(4 batches, 21 g for each batch). The reaction mixture was diluted with 1000 mL brine and 1000 mL ethyl acetate, filtered through a pad of silica gel and quartz sand, washed with 3000 mL ethyl acetate. Then separated, the water layer was extracted with ethyl acetate (2000 mL×3). The combined organic layers were washed with brine (1000 mL×3), dried over Na₂SO₄, filtered and concentrated under reduced pressure to give a residue. The residue was purified by flash silica gel chromatography to afford the title compound. MS (ESI): m/z=238.9 [M+H]⁺.

Ethyl 5-(bromomethyl)-4-fluorobenzo[b]thiophene-2-carboxylate. Solution 1: ethyl 4-fluoro-5-methylbenzo[b]thiophene-2-carboxylate (the mixture with ethyl 4-fluoro-7-methylbenzo[b]thiophene-2-carboxylate) (9.5 g, 39.9 mmol) and N-Bromosuccinimide (8.5 g, 47.8 mmol) in Acetonitrile (285 mL). The solution 1 was pumped by Pump 1 S1, P1, 5.338 mL/min to flow reactor 1 FLR1, FEP, Coils reactor, 3.175 (⅛″) mm, 80.0 mL, 45° C. The residence time of flow reactor 1 was FLR1, 15 min. The mixture was collected with a bottle. The reaction mixture was quenched by addition Na₂SO₃ 50 mL, and then extracted with Ethyl acetate 150 mL (50 mL×3). The combined organic layers were washed with brine 90 mL (30 mL×3), dried over Na₂SO₄, filtered and concentrated under reduced pressure to give a residue. The residue was purified by flash silica gel chromatography to give ethyl 5-(bromomethyl)-4-fluorobenzo[b]thiophene-2-carboxylate. MS (ESI): m/z=316.7/318.8 [M+H]⁺.

Ethyl 5-((diethoxyphosphoryl)methyl)-4-fluorobenzo[b]thiophene-2-carboxylate. To a solution of ethyl 5-(bromomethyl)-4-fluorobenzo[b]thiophene-2-carboxylate (11.0 g, 34.7 mmol) in DMF (105 mL) was added triethyl phosphite (8.6 g, 52.0 mmol). The mixture was stirred at 100° C. for 12 hr. The reaction mixture was diluted with H₂O 40 mL and extracted with Ethyl acetate 120 mL (40 mL×3). The combined organic layers were washed with brine 60 mL (20 mL×3), dried over Na₂SO₄, filtered and concentrated under reduced pressure to give a residue. The residue was purified by flash silica gel chromatography to give ethyl 5-((diethoxyphosphoryl)methyl)-4-fluorobenzo[b]thiophene-2-carboxylate. MS (ESI): m/z=374.9 [M+H]⁺.

Ethyl 5-((diethoxyphosphoryl)fluoromethyl)-4-fluorobenzo[b]thiophene-2-carboxylate. To a solution of ethyl 5-((diethoxyphosphoryl)methyl)-4-fluorobenzo[b]thiophene-2-carboxylate (5.0 g, 13.4 mmol) in Tetrahydrofuran (60.0 mL) was added Sodium bis(trimethylsilyl)amide (1.0 mol/L, 13.4 mL, 13.4 mmol) at −78° C. under N₂. The mixture was stirred at −78° C. for 1 hr under N₂. N-Fluorobenzenesulfonimide, 97% (4.21 g, 13.4 mmol) in Tetrahydrofuran (30.0 mL) was added to the solution. The mixture was stirred at −78° C. for 1 hr under N₂. The mixture was quenched by addition into sat. NH₄Cl 30 mL at 0° C. Then extracted with ethyl acetate (50 mL×3). The combined organic layers were washed with brine (50 mL×3), dried over Na₂SO₄, filtered and concentrated under reduced pressure to give a residue. The residue was purified by flash silica gel chromatography to give product. MS (ESI): m/z=393.1 [M+H]⁺.

Ethyl (R and S)-5-((diethoxyphosphoryl)fluoromethyl)-4-fluorobenzo[b]thiophene-2-carboxylate (B8.1). A mixture of ethyl 5-((diethoxyphosphoryl)fluoromethyl)-4-fluorobenzo[b]thiophene-2-carboxylate (19.7 g, 0.0502 mol) and lithium hydroxide monohydrate (4.64 g, 0.110 mol) in EtOH (240 mL)/H₂O (80.0 mL) stirred at 20° C. for 0.5 hr. The mixture was quenched by addition of 6M HCl to adjust pH<3, then concentrated under reduced pressure to remove solvent. Then diluted with H₂O 50 mL and extracted with ethyl acetate (100 mL×3). The combined organic layers were dried over Na₂SO₄, filtered and concentrated under reduced pressure to give a residue. The residue filtrate was purified by prep-HPLC (TFA condition, column: Phenomenex luna C18 (250*70 mm, 15 um); mobile phase: [A: H₂O (0.1% TFA); B: ACN]; B %: 25.00%-55.00%, 25.00 min; flow rate: 130.00 ml/min) to give the title compound. MS (ESI): m/z=365.0 [M+H]⁺.

(R or S)-5-((diethoxyphosphoryl)fluoromethyl)-4-fluorobenzo[b]thiophene-2-carboxylic acid (B8.2) and (R or S)-5-((diethoxyphosphoryl)fluoromethyl)-4-fluorobenzo[b]thiophene-2-carboxylic acid (B8.). Ethyl (R and S)-5-((diethoxyphosphoryl)fluoromethyl)-4-fluorobenzo[b]thiophene-2-carboxylate (B8.1) was further separated by SFC (column: DAICEL CHIRALPAK AD (250 mm*30 mm, 10 um); mobile phase: [A: CO₂; B: IPA(0.1% NH₃H₂O)]; B %: 18.00%-18.00%, 8.00 min)(LCMS CB39-28-P1B2) to isolate (R or S)-5-((diethoxyphosphoryl)fluoromethyl)-4-fluorobenzo[b]thiophene-2-carboxylic acid (B8.2, peak 1) and (R or S)-5-((diethoxyphosphoryl)fluoromethyl)-4-fluorobenzo[b]thiophene-2-carboxylic acid (B8.3, peak 2). MS (ESI): m/z=365.0 [M+H]⁺. ¹H NMR (400 MHz, CHLOROFORM M-d) δ 7.96 (s, 1H), 7.71-7.62 (m, 2H), 6.33-6.16 (m, 1H), 4.41-4.20 (m, 4H), 1.45-1.38 (m, 6H).

### Preparation of (R or S)-allyl 5-((diethoxyphosphoryl)fluoromethyl)-4-fluorobenzo[b]thiophene-2-carboxylate (B8.4)

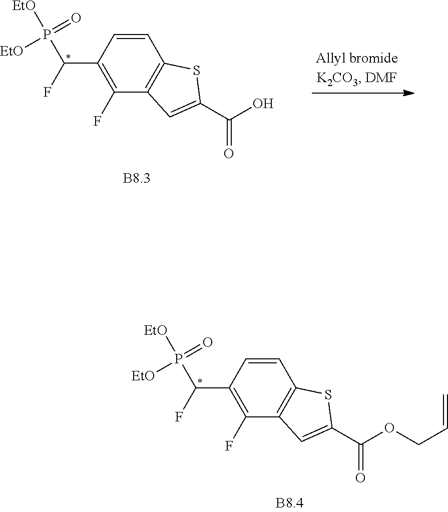 [SMILES: CCOP(=O)(OCC)C(F)c1ccc2sc(C(=O)O)cc2c1F ; C=CCOC(=O)c1cc2c(F)c(C(F)P(=O)(OCC)OCC)ccc2s1]

(R or S)-allyl 5-((diethoxyphosphoryl)fluoromethyl)-4-fluorobenzo[b]thiophene-2-carboxylate (B8.4). 5-[(R)-diethoxyphosphoryl(fluoro)methyl]-4-fluoro-benzothiophene-2-carboxylic acid (B8.3, 560 mg, 0.00154 mol), POTASSIUM CARBONATE (0.234 g, 0.00169 mol) and ALLYL BROMIDE (0.133 mL, 0.00154 mol) were suspended in DMF (11.2 mL) and allowed to stir at room temperature for 2 hours. After the allotted time, the reaction was quenched with water, extracted with ethyl acetate (2×15 mL), dried over magnesium sulfate, filtered and concentrated under reduced pressure to afford the title compound, which was used as is in subsequent reactions. ES/MS: m/z=404.9 [M+H]⁺.

### Preparation of 5-((diethoxyphosphoryl)difluoromethyl)-4-fluorobenzo[b]thiophene-2-carboxylic acid (B8.5)

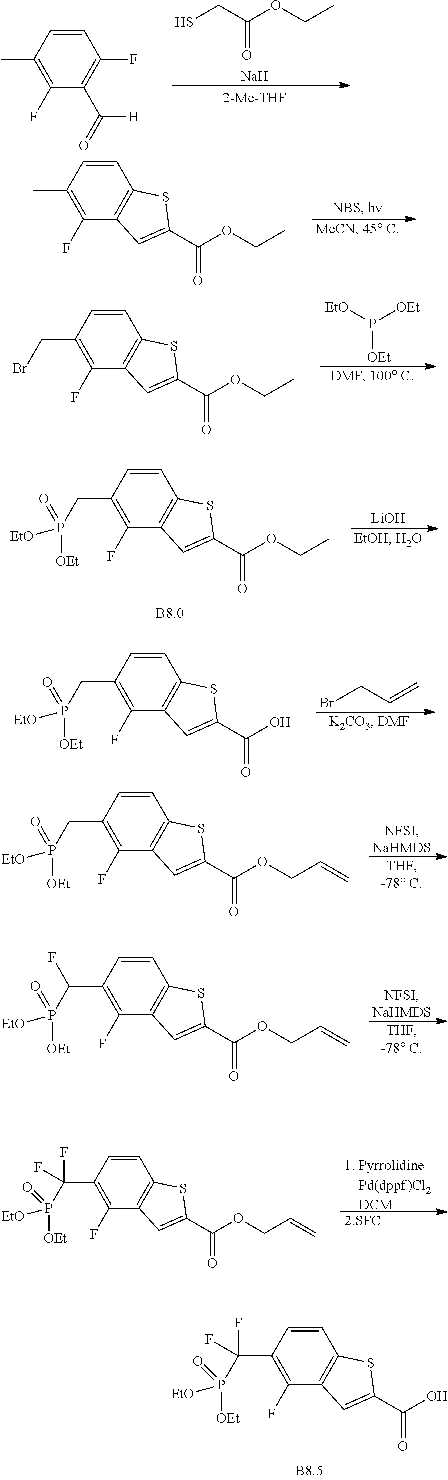

Ethyl 4-fluoro-5-methylbenzo[b]thiophene-2-carboxylate. To a solution of sodium hydride (60.0%, 3.8 g, 96.1 mmol) in 2-MeTHF (100 mL) was added ethyl 2-sulfanylacetate (10.0 g, 83.3 mmol) at 25° C. under N₂. The mixture was stirred at 20° C. for 1 hr. Then 2,6-difluoro-3-methyl-benzaldehyde (10.0 g, 64.0 mmol) in 2-MeTHF (50.0 mL) was added to the solution at 20° C. The mixture was stirred at 20° C. for 2 hr under N₂. To the mixture was added sat. aq. NH₄Cl (200 mL) at 25° C., and extracted with EtOAc 450 mL (150 mL×3). The combined organics were washed with brine 200 mL (100 mL×2), dried over Na₂SO₄, filtered and concentrated under reduced pressure to give a residue. The residue was purified by flash silica gel chromatography (120 g Silica Flash Column, Eluent of 0-5% Ethyl acetate/Petroleum ether gradient) to give ethyl 4-fluoro-5-methylbenzo[b]thiophene-2-carboxylate as a mixture with ethyl 4-fluoro-7-methylbenzo[b]thiophene-2-carboxylate. ¹H NMR (400 MHz, CHLOROFORM-d) δ 8.17 (s, 1H), 8.11 (s, 1H), 7.51 (d, J=8.3 Hz, 1H), 7.27 (s, 1H), 7.19 (dd, J=4.8, 7.9 Hz, 1H), 6.99 (dd, J=8.1, 9.8 Hz, 1H), 4.47-4.38 (m, 3H), 2.52 (s, 3H), 1.46-1.44 (m, 1H), 1.44-1.40 (m, 3H). MS (ESI): m/z=238.9 [M+H]⁺

Ethyl 5-(bromomethyl)-4-fluorobenzo[b]thiophene-2-carboxylate. Solution 1: ethyl 4-fluoro-5-methylbenzo[b]thiophene-2-carboxylate (the mixture with ethyl 4-fluoro-7-methylbenzo[b]thiophene-2-carboxylate) (9.5 g, 39.9 mmol) and N-Bromosuccinimide (8.5 g, 47.8 mmol) in acetonitrile (285 mL). “Solution 1” was pumped at a rate 5.338 mL/min to flow reactor 1 (FEP, Coils reactor, 3.175 (⅛″) mm, 80.0 mL, 45° C.). The residence time of flow reactor 1 was 15 min. The mixture was collected and Na₂SO₃ 50 mL was added, and then extracted with ethyl acetate 150 mL (50 mL×3). The combined organics were washed with brine (30 mL×3), dried over Na₂SO₄, filtered, and concentrated under reduced pressure to give a residue. The residue was purified by flash silica gel chromatography (40 g Silica Flash Column, Eluent of 0-20% Ethyl acetate/Petroleum ether gradient) to give ethyl 5-(bromomethyl)-4-fluorobenzo[b]thiophene-2-carboxylate (as a mixture with ethyl 7-(bromomethyl)-4-fluorobenzo[b]thiophene-2-carboxylate). ¹H NMR (400 MHz, CHLOROFORM-d) δ 8.21 (s, 1H), 8.19 (s, 1H), 8.15 (s, 1H), 7.48-7.44 (m, 1H), 7.08-7.02 (m, 1H), 4.72 (s, 2H), 4.50-4.40 (m, 4H), 1.48-1.41 (m, 6H). MS (ESI): m/z=316.7/318.8 [M+H]⁺

Ethyl 5-((diethoxyphosphoryl)methyl)-4-fluorobenzo[b]thiophene-2-carboxylate. To a solution of ethyl 5-(bromomethyl)-4-fluorobenzo[b]thiophene-2-carboxylate (as a mixture with ethyl 7-(bromomethyl)-4-fluorobenzo[b]thiophene-2-carboxylate) (11.0 g, 34.7 mmol) in DMF (105 mL) was added triethyl phosphite (8.6 g, 52.0 mmol). The mixture was stirred at 100° C. for 12 hr. The mixture was then diluted with H₂O 40 mL and extracted with Ethyl acetate 120 mL (40 mL×3). The combined organics were washed with brine 60 mL (20 mL×3), dried over Na₂SO₄, filtered, and concentrated under reduced pressure to give a residue. The residue was purified by flash silica gel chromatography (40 g Silica Flash Column, Eluent of 0-40% Ethyl acetate/Petroleum ether gradient) to give ethyl 5-((diethoxyphosphoryl)methyl)-4-fluorobenzo[b]thiophene-2-carboxylate as a mixture with ethyl 7-((diethoxyphosphoryl)methyl)-4-fluorobenzo[b]thiophene-2-carboxylate. ¹H NMR (400 MHz, CHLOROFORM-d) δ 8.15 (s, 1H), 8.02 (s, 1H), 7.59 (d, J=8.4 Hz, 1H), 7.48-7.40 (m, 1H), 7.06 (t, J=8.9 Hz, 1H), 4.42 (q, J=7.1 Hz, 3H), 4.19-4.10 (m, 1H), 4.09-3.99 (m, 6H), 3.40-3.27 (m, 3H), 1.42 (t, J=7.2 Hz, 5H), 1.22 (t, J=7.1 Hz, 6H). MS (ESI): m/z=374.9 [M+H]⁺

5-((diethoxyphosphoryl)methyl)-4-fluorobenzo[b]thiophene-2-carboxylic acid. To a solution of ethyl 5-((diethoxyphosphoryl)methyl)-4-fluorobenzo[b]thiophene-2-carboxylate (as a mixture with ethyl 7-((diethoxyphosphoryl)methyl)-4-fluorobenzo[b]thiophene-2-carboxylate) (3.00 g, 8.0 mmol) in EtOH (57.0 mL)/H₂O (19.0 mL) was added lithium hydroxide monohydrate (0.7 g, 17.6 mmol). The mixture was stirred at 20° C. for 0.5 hr. The mixture was adjusted pH to 3-4 with 1M aq. HCl and then extracted with ethyl acetate (50 mL×3). The combined organic layers were dried over Na2SO4, filtered and concentrated under reduced pressure to give the crude 5-((diethoxyphosphoryl)methyl)-4-fluorobenzo[b]thiophene-2-carboxylic acid as a mixture with 7-((diethoxyphosphoryl)methyl)-4-fluorobenzo[b]thiophene-2-carboxylic acid which was used in the next step without further purification. MS (ESI): m/z=346.8 [M+H]+

Allyl 5-((diethoxyphosphoryl)methyl)-4-fluorobenzo[b]thiophene-2-carboxylate. To a solution of 5-((diethoxyphosphoryl)methyl)-4-fluorobenzo[b]thiophene-2-carboxylic acid as a mixture with 7-((diethoxyphosphoryl)methyl)-4-fluorobenzo[b]thiophene-2-carboxylic acid (2.7 g, 8.0 mmol) in dimethylformamide (22.7 mL) was added potassium carbonate (3.3 g, 24.1 mmol) and 3-bromoprop-1-ene (1.9 g, 16.1 mmol). The mixture was stirred at 20° C. for 12 hr. The residue was purified by flash silica gel chromatography (12 g Flash Silica Column, Eluent of 0-40% Ethyl acetate/Petroleum ether gradient) to give allyl 5-((diethoxyphosphoryl)methyl)-4-fluorobenzo[b]thiophene-2-carboxylate as a mixture with allyl 7-((diethoxyphosphoryl)methyl)-4-fluorobenzo[b]thiophene-2-carboxylate. MS (ESI): m/z=386.9 [M+H]+

Allyl 5-((diethoxyphosphoryl)fluoromethyl)-4-fluorobenzo[b]thiophene-2-carboxylate. To a solution of allyl 5-((diethoxyphosphoryl)methyl)-4-fluorobenzo[b]thiophene-2-carboxylate as a mixture with allyl 7-((diethoxyphosphoryl)methyl)-4-fluorobenzo[b]thiophene-2-carboxylate (2.4 g, 6.2 mmol) in tetrahydrofuran (30.0 mL) was added sodium bis(trimethylsilyl)amide (1.0 mol/L, 9.3 mL, 9.3 mmol) at −78° C. under N₂. The mixture was stirred at −78° C. for 1 hr under N₂. Then N-Fluorobenzenesulfonimide, 97% (3.9 g, 12.4 mmol) in tetrahydrofuran (10.0 mL) was added to the solution. The mixture was stirred at −78° C. for 1 hr under N₂. To the mixture was then added into sat. aq. NH₄Cl 30 mL at 0° C. The mixture was then warmed to room temperature and extracted with ethyl acetate (30 mL×3). The combined organic layers were washed with brine (20 mL×3), dried over Na₂SO₄, filtered, and concentrated under reduced pressure to give a residue. The residue was purified by flash silica gel chromatography (20 g Silica Flash Column, Eluent of 0-20% Ethyl acetate/Petroleum ether gradient) to give allyl 5-((diethoxyphosphoryl)fluoromethyl)-4-fluorobenzo[b]thiophene-2-carboxylate as a mixture with allyl 7-((diethoxyphosphoryl)fluoromethyl)-4-fluorobenzo[b]thiophene-2-carboxylate. MS (ESI): m/z=404.9 [M+H]+

Allyl 5-((diethoxyphosphoryl)difluoromethyl)-4-fluorobenzo[b]thiophene-2-carboxylate. To a solution of allyl 5-((diethoxyphosphoryl)fluoromethyl)-4-fluorobenzo[b]thiophene-2-carboxylate as a mixture with allyl 7-((diethoxyphosphoryl)fluoromethyl)-4-fluorobenzo[b]thiophene-2-carboxylate (1.1 g, 2.9 mmol) in tetrahydrofuran (17.0 mL) was added sodium bis(trimethylsilyl)amide (1.0 mol/L, 3.5 mL, 3.5 mmol) at −78° C. under N₂. The mixture was stirred at -78° C. for 1 hr under N₂. N-Fluorobenzenesulfonimide, 97% (1.1 g, 3.5 mmol) in tetrahydrofuran (8.0 mL) was added to the solution. The mixture was stirred at −78° C. for 1 hr under N₂. The reaction mixture was then added into sat. NH₄Cl 10 mL at 0° C., warmed to room temperature, and then extracted with ethyl acetate (20 mL×3). The combined organic layers were washed with brine (10 mL×3), dried over Na₂SO₄, filtered, and concentrated under reduced pressure to give a residue. The residue was purified by flash silica gel chromatography (20 g Silica Flash Column, Eluent of 0-20% Ethyl acetate/Petroleum ether gradient) to give allyl 5-((diethoxyphosphoryl)difluoromethyl)-4-fluorobenzo[b]thiophene-2-carboxylate as a mixture with allyl 7-((diethoxyphosphoryl)difluoromethyl)-4-fluorobenzo[b]thiophene-2-carboxylate. MS (ESI): m/z=422.9 [M+H]+

5-((diethoxyphosphoryl)difluoromethyl)-4-fluorobenzo[b]thiophene-2-carboxylic acid (B8.5). To a solution of allyl 5-((diethoxyphosphoryl)difluoromethyl)-4-fluorobenzo[b]thiophene-2-carboxylate as a mixture with allyl 7-((diethoxyphosphoryl)difluoromethyl)-4-fluorobenzo[b]thiophene-2-carboxylate (2.2 g, 5.2 mmol, 1 eq) in DCM (40 mL) was added pyrrolidine (370.4 mg, 5.2 mmol, 434.81 μL, 1 eq) and Pd(dppf)Cl₂ (381.1 mg, 520.8 μmol, 0.1 eq) at 25° C. under N₂. The mixture was stirred at 25° C. for 1.5 hr. The mixture was then concentrated under reduced pressure to give a residue. The residue was purified by prep-HPLC (TFA condition; column: Phenomenex luna C18 (250*70 mm, 15 um); mobile phase: [H₂O (0.1% TFA)-ACN]; gradient: 20%-65% B over 25.0 min to give 5-((diethoxyphosphoryl)difluoromethyl)-4-fluorobenzo[b]thiophene-2-carboxylic acid which was further separated by SFC (column: DAICEL CHIRALPAK AD (250 mm*30 mm, 10 um); mobile phase: [CO₂-EtOH (0.1% NH3H2O)]; B %: 30%, isocratic elution mode) to afford 5-((diethoxyphosphoryl)difluoromethyl)-4-fluorobenzo[b]thiophene-2-carboxylic acid (B8.5). 1H NMR (400 MHz, DMSO-d6) δ 8.04-7.98 (m, 2H), 7.55 (t, J=7.6 Hz, 1H), 7.34-6.94 (m, 1H), 4.23-4.10 (m, 4H), 1.27-1.21 (m, 6H).

### Preparation of 5-((bis(((S)-1-oxo-1-propoxypropan-2-yl)amino)phosphoryl)fluoromethyl)-4-fluorobenzo[b]thiophene-2-carboxylic acid (B9.1)

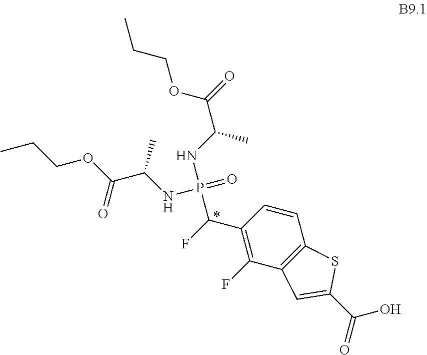 [SMILES: CCCOC(=O)[C@H](C)NP(=O)(N[C@@H](C)C(=O)OCCC)C(F)c1ccc2sc(C(=O)O)cc2c1F]

(R or S)-5-((bis(((S)-1-oxo-1-propoxypropan-2-yl)amino)phosphoryl)fluoromethyl)-4-fluorobenzo[b]thiophene-2-carboxylic acid (B9.1). B9.1 was prepared analogously to B6.3, beginning with B8.4 instead of B6.1. ES/MS: m/z=535.2 [M+H]⁺.

### Preparation of Allyl 4-fluoro-5-(fluoro((((S)-1-oxo-1-propoxypropan-2-yl)amino)(phenoxy)phosphoryl)methyl)benzo[b]thiophene-2-carboxylate (B10.2)

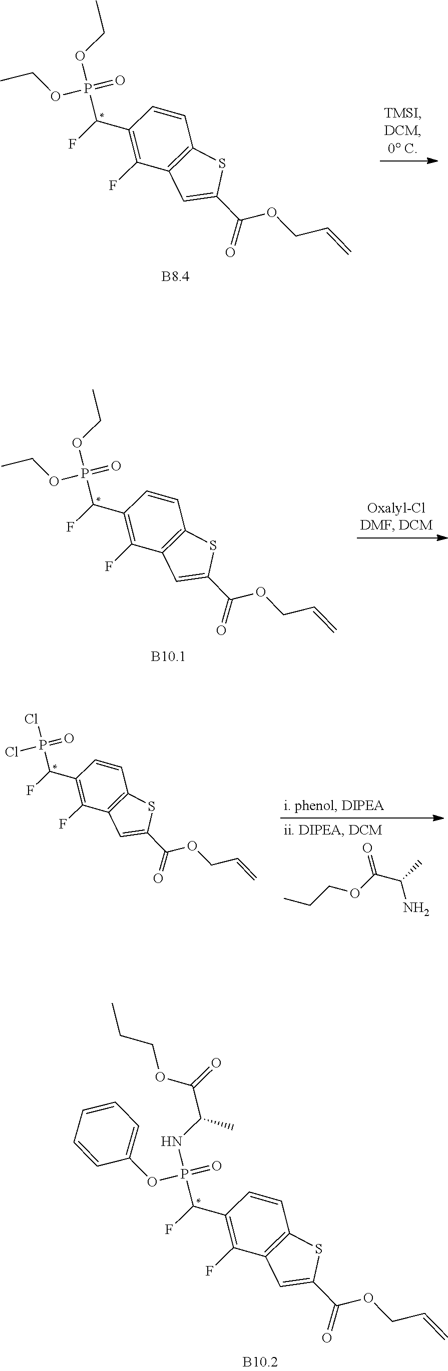

((2-((allyloxy)carbonyl)-4-fluorobenzo[b]thiophen-5-yl)fluoromethyl)phosphonic acid (B10.1). B8.4 (127 mg, 0.31 mmol) was suspended in DCM (0.3 mL, 110 mM). The solution was cooled to 0° C. in an ice bath and allowed to stir for 5 minutes. 1 M iodotrimethylsilane in DCM (2.51 mL, 8 equiv, 2.5 mmol) was then added drop-wise. After stirring for 30 minutes at 0 TC, the reaction mixture was concentrated and purified directly via reverse phase column chromatography to afford the title compound. MS (ESI): m/z=346.9 [M−H]⁻.

Allyl 5-((dichlorophosphoryl)fluoromethyl)-4-fluorobenzo[b]thiophene-2-carboxylate. ((2-((allyloxy)carbonyl)-4-fluorobenzo[b]thiophen-5-yl)fluoromethyl)phosphonic acid (B10.1, 34 mg, 97 mmol) was suspended in DCM (0.28 mL, 0.31 mM). The solution was cooled to 0° C. in an ice bath and allowed to stir for 5 minutes. Oxalyl chloride (0.29 mmol, 3 equiv) was then added drop-wise. The solution was allowed to stir at 0° C. for 5 additional minutes before being warmed to room temperature and stirred for an additional 30 minutes. At this point, the solution was concentrated under reduced pressure and used as is in subsequent reactions.

Allyl 4-fluoro-5-(fluoro((((S)-1-oxo-1-propoxypropan-2-yl)amino)(phenoxy)phosphoryl)methyl)benzo[b]thiophene-2-carboxylate (B10.2). Allyl 5-((dichlorophosphoryl)fluoromethyl)-4-fluorobenzo[b]thiophene-2-carboxylate (36 mg, 94 mmol) was suspended in DCM (0.6 mL, 120 mM). The solution was cooled to 0° C. in an ice bath and allowed to stir for 5 minutes. Phenol (7.9 mg, 0.9 equiv, 84 mmol) was then added drop-wise, followed by drop-wise addition of DIPEA (0.17 mL, 10 equiv, 0.94 mmol). The reaction was allowed to stir for 30 minutes at 0° C. Propyl L-alaninate hydrochloride (31 mg, 2.5 equiv, 0.23 mmol) was then added drop-wise in 0.2 mL of DCM. The mixture was stirred at 0° C. for 15 minutes, then concentrated under reduced pressure and purified directly via reverse phase column chromatography to afford the title compound. MS (ESI): m/z=538.0 [M+H]⁺.

### Preparation of 4-fluoro-5-(fluoro((((S)-1-oxo-1-propoxypropan-2-yl)amino)(phenoxy)phosphoryl)methyl)benzo[b]thiophene-2-carboxylic acid (B10.3)

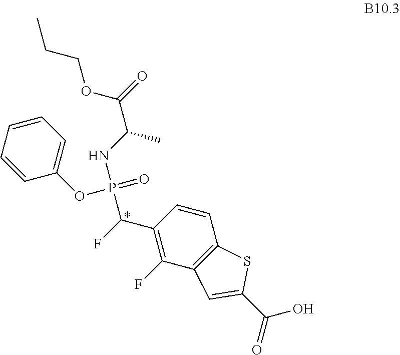 [SMILES: CCCOC(=O)[C@H](C)NP(=O)(Oc1ccccc1)C(F)c1ccc2sc(C(=O)O)cc2c1F]

4-Fluoro-5-(fluoro((((S)-1-oxo-1-propoxypropan-2-yl)amino)(phenoxy)phosphoryl)methyl)benzo[b]thiophene-2-carboxylic acid (B10.3). B10.3 was prepared analogously to B6.3 using B10.2 as the starting material. MS (ESI): m/z=498.0 [M+H]⁺.

### Preparation of perfluorophenyl 5-(fluoro((((S)-1-oxo-1-propoxypropan-2-yl)amino)(phenoxy)phosphoryl)methyl)benzo[b]thiophene-2-carboxylate (B11.1) and perfluorophenyl 5-(fluoro((((S)-1-oxo-1-propoxypropan-2-yl)amino)(phenoxy)phosphoryl)methyl)benzo[b]thiophene-2-carboxylate (B11.2)

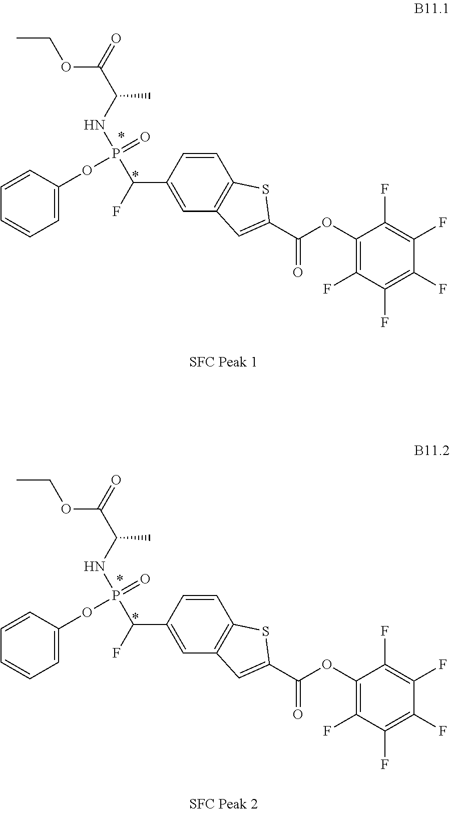 [SMILES: CCOC(=O)[C@H](C)NP(=O)(Oc1ccccc1)C(F)c1ccc2sc(C(=O)Oc3c(F)c(F)c(F)c(F)c3F)cc2c1 ; CCOC(=O)[C@H](C)NP(=O)(Oc1ccccc1)C(F)c1ccc2sc(C(=O)Oc3c(F)c(F)c(F)c(F)c3F)cc2c1]

Perfluorophenyl 5-(fluoro((((S)-1-oxo-1-propoxypropan-2-yl)amino)(phenoxy)phosphoryl)methyl)benzo[b]thiophene-2-carboxylate (B11.1) and perfluorophenyl 5-(fluoro((((S)-1-oxo-1-propoxypropan-2-yl)amino)(phenoxy)phosphoryl)methyl)benzo[b]thiophene-2-carboxylate (B11.2). B11.1 and B11.2 were prepared analogously to B5.7 and B5.8 using ethyl L-alanine. MS (ESI): m/z=632.1 [M+H]⁺.

### Preparation of perfluorophenyl 5-(fluoro((((S)-1-isopropoxy-1-oxopropan-2-yl)amino)(phenoxy)phosphoryl)methyl)benzo[b]thiophene-2-carboxylate (B12.1) and perfluorophenyl 5-(fluoro((((S)-1-isopropoxy-1-oxopropan-2-yl)amino)(phenoxy)phosphoryl)methyl)benzo[b]thiophene-2-carboxylate (B12.2)

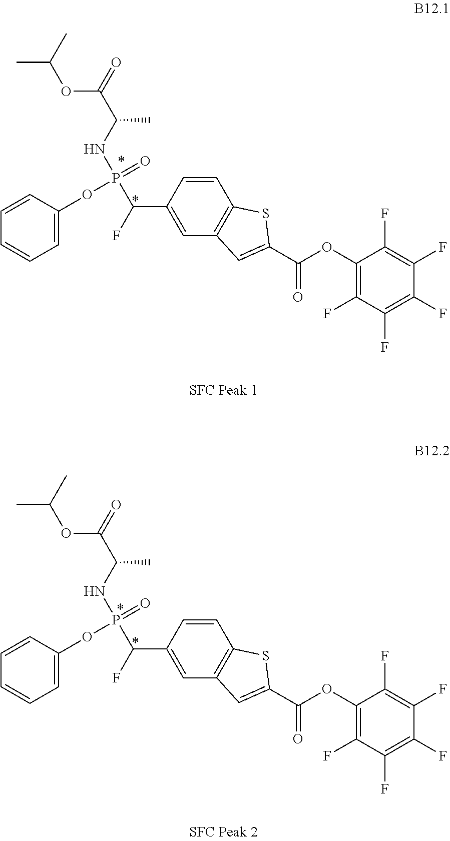 [SMILES: CC(C)OC(=O)[C@H](C)NP(=O)(Oc1ccccc1)C(F)c1ccc2sc(C(=O)Oc3c(F)c(F)c(F)c(F)c3F)cc2c1 ; CC(C)OC(=O)[C@H](C)NP(=O)(Oc1ccccc1)C(F)c1ccc2sc(C(=O)Oc3c(F)c(F)c(F)c(F)c3F)cc2c1]

Perfluorophenyl 5-(fluoro((((S)-1-isopropoxy-1-oxopropan-2-yl)amino)(phenoxy)phosphoryl)methyl)benzo[b]thiophene-2-carboxylate (B12.1) and perfluorophenyl 5-(fluoro((((S)-1-isopropoxy-1-oxopropan-2-yl)amino)(phenoxy)phosphoryl)methyl)benzo[b]thiophene-2-carboxylate (B12.2). B12.1 and B12.2 were prepared analogously to B5.7 and B5.8 using isopropyl L-alaninate. MS (ESI): m/z=646.1 [M+H]⁺.

### Preparation of perfluorophenyl 5-(fluoro((((S)-1-methoxy-1-oxopropan-2-yl)amino)(phenoxy)phosphoryl)methyl)benzo[b]thiophene-2-carboxylate (B13.1) and perfluorophenyl 5-(fluoro((((S)-1-methoxy-1-oxopropan-2-yl)amino)(phenoxy)phosphoryl)methyl)benzo[b]thiophene-2-carboxylate (B13.2)

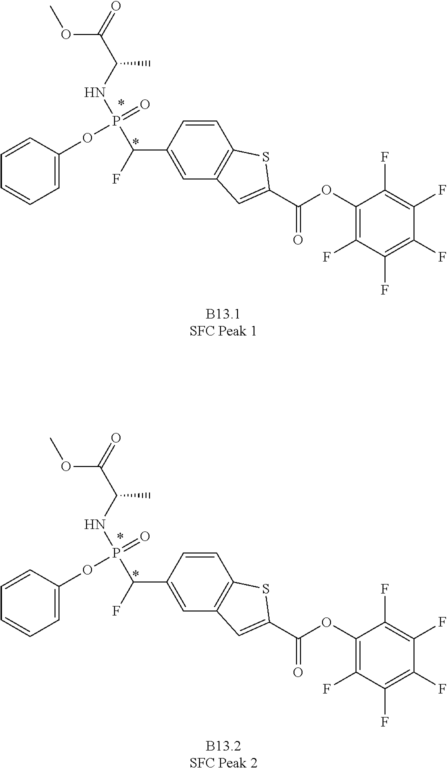 [SMILES: COC(=O)[C@H](C)NP(=O)(Oc1ccccc1)C(F)c1ccc2sc(C(=O)Oc3c(F)c(F)c(F)c(F)c3F)cc2c1 ; COC(=O)[C@H](C)NP(=O)(Oc1ccccc1)C(F)c1ccc2sc(C(=O)Oc3c(F)c(F)c(F)c(F)c3F)cc2c1]

perfluorophenyl 5-(fluoro((((S)-1-methoxy-1-oxopropan-2-yl)amino)(phenoxy)phosphoryl)methyl)benzo[b]thiophene-2-carboxylate (B13.1) and perfluorophenyl 5-(fluoro((((S)-1-methoxy-1-oxopropan-2-yl)amino)(phenoxy)phosphoryl)methyl)benzo[b]thiophene-2-carboxylate (B13.2). B13.1 and B13.2 were prepared analogously to B5.7 and B5.8 using methyl L-alaninate. MS (ESI): m/z=618.1 [M+H]⁺.

### Preparation of 2-(((2S,3R)-1-((S)-2-(5-((diethoxyphosphoryl)difluoromethyl)benzo[b]thiophene-2-carboxamido)-3,3-dimethylbutanoyl)-2-((R)-2-phenylmorpholine-4-carbonyl)pyrrolidin-3-yl)oxy)acetic acid (C1.5)

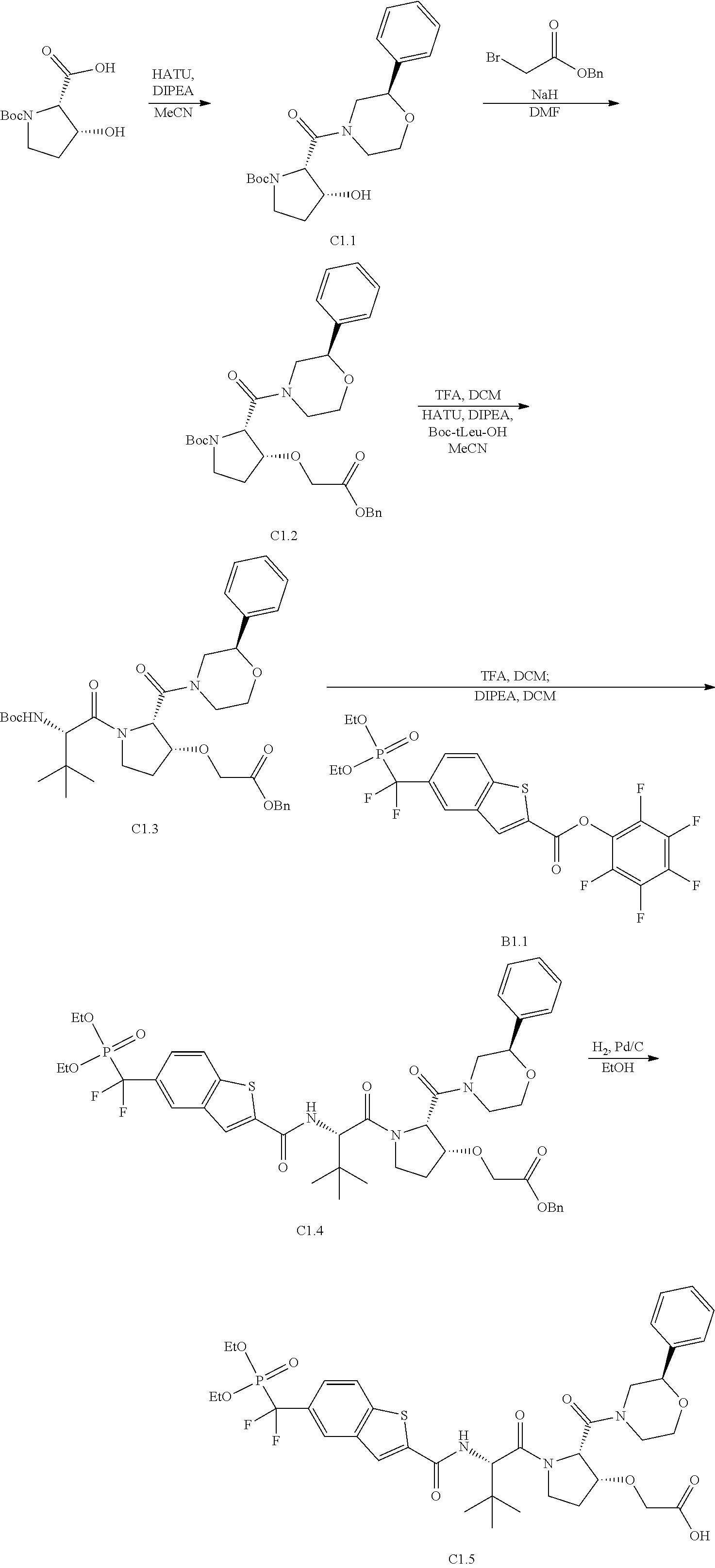

tert-butyl (2S,3R)-3-hydroxy-2-((R)-2-phenylmorpholine-4-carbonyl)pyrrolidine-1-carboxylate (C1.1). To a stirred solution of (2S,3R)-1-tert-butoxycarbonyl-3-hydroxy-pyrrolidine-2-carboxylic acid (2.0 g, 8.7 mmol) and (2R)-2-phenylmorpholine (1.4 g, 8.7 mmol) in MeCN (30 mL) was added DIPEA (3.1 mL, 17 mmol) and HATU (3.1 g, 8.2 mmol). The mixture was stirred for 30 minutes, then concentrated. The crude residue was purified by silica gel flash column chromatography to afford tert-butyl (2S,3R)-3-hydroxy-2-((R)-2-phenylmorpholine-4-carbonyl)pyrrolidine-1-carboxylate. ES/MS: m/z=376.9 [M+H]⁺. ¹H NMR (500 MHz, DMSO-d6) δ 7.51-7.33 (m, 5H), 5.49-5.23 (m, 1H), 4.90-4.62 (m, 1H), 4.56-4.20 (m, 3H), 4.15-3.93 (m, 2H), 3.78-3.40 (m, 2H), 3.32-2.97 (m, 2H), 2.89-2.57 (m, 1H), 1.98-1.69 (m, 2H), 1.47-1.27 (m, 9H).

tert-butyl (2S,3R)-3-(2-(benzyloxy)-2-oxoethoxy)-2-((R)-2-phenylmorpholine-4-carbonyl)pyrrolidine-1-carboxylate (C1.2). Sodium hydride (60% dispersion in mineral oil, 460 mg, 12 mmol) was added to a solution of tert-butyl (2S,3R)-3-hydroxy-2-((R)-2-phenylmorpholine-4-carbonyl)pyrrolidine-1-carboxylate (3.0 g, 8.0 mmol) in DMF (35 mL) at 0° C. After stirring for 30 minutes, benzyl bromoacetate (1.4 mL, 8.8 mmol) was added. The mixture was allowed to warm to room temperature and stir overnight. Sat. Aq. NH₄Cl was then added, and the mixture was diluted with EtOAc. The mixture was transferred to a separatory funnel, and the organic layer was washed with 10% aq. LiCl then brine, dried over MgSO₄, filtered, and concentrated. Purification by silica gel flash column chromatography afforded tert-butyl (2S,3R)-3-(2-(benzyloxy)-2-oxoethoxy)-2-((R)-2-phenylmorpholine-4-carbonyl)pyrrolidine-1-carboxylate. ES/MS: m/z=524.9 [M+H]⁺. ¹H NMR (500 MHz, DMSO-d6) δ 7.49-7.25 (m, 10H), 5.29-5.12 (m, 2H), 5.01-4.76 (m, 1H), 4.52-3.88 (m, 8H), 3.72-3.58 (m, 1H), 3.58-3.46 (m, 1H), 3.35-3.16 (m, 2H), 2.27-2.10 (m, 1H), 2.07-1.93 (m, 1H), 1.57-1.33 (m, 9H).

benzyl 2-(((2S,3R)-1-((S)-2-((tert-butoxycarbonyl)amino)-3,3-dimethylbutanoyl)-2-((R)-2-phenylmorpholine-4-carbonyl)pyrrolidin-3-yl)oxy)acetate (C1.3). tert-butyl (2S,3R)-3-(2-(benzyloxy)-2-oxoethoxy)-2-((R)-2-phenylmorpholine-4-carbonyl)pyrrolidine-1-carboxylate (2.1 g, 4.0 mmol) was suspended in DCM/TFA (5:1, 20 mL). After stirring for 1 hour, the mixture was concentrated then resuspended in MeCN (20 mL). (2S)-2-(tert-butoxycarbonylamino)-3,3-dimethyl-butanoic acid (970 mg, 4.2 mmol), DIPEA, (3.6 mL, 20.0 mmol), and HATU (1.4 g, 3.7 mmol) were then added. After stirring for 30 min, the mixture was concentrated and purified by silica gel flash column chromatography to afford benzyl 2-(((2S,3R)-1-((S)-2-((tertbutoxycarbonyl)amino)-3,3-dimethylbutanoyl)-2-((R)-2-phenylmorpholine-4-carbonyl)pyrrolidin-3-yl)oxy)acetate. ES/MS: m/z=637.9 [M+H]⁺. ¹H NMR (500 MHz, DMSO-d6) δ 7.52-7.25 (m, 10H), 6.09-5.58 (m, 1H), 5.31-5.05 (m, 3H), 4.63-3.90 (m, 10H), 3.86-3.57 (m, 3H), 2.30-2.12 (m, 2H), 1.09-0.90 (m, 9H).

benzyl 2-(((2S,3R)-1-((S)-2-(5-((diethoxyphosphoryl)difluoromethyl)benzo[b]thiophene-2-carboxamido)-3,3-dimethylbutanoyl)-2-((R)-2-phenylmorpholine-4-carbonyl)pyrrolidin-3-yl)oxy)acetate (C1.4). benzyl 2-(((2S,3R)-1-((S)-2-((tert-butoxycarbonyl)amino)-3,3-dimethylbutanoyl)-2-((R)-2-phenylmorpholine-4-carbonyl)pyrrolidin-3-yl)oxy)acetate (340 mg, 0.53 mmol) was suspended in DCM/TFA (5:1, 2.0 mL). After stirring for 1 hour, the mixture was concentrated and resuspended in DCM (5 mL). (2,3,4,5,6-pentafluorophenyl) 5-[diethoxyphosphoryl(difluoro)methyl]benzothiophene-2-carboxylate (280 mg, 0.53 mmol) B1.1 and DIPEA (1.4 mL, 8.0 mmol) were then added. After stirring overnight, the mixture was concentrated and purified by silica gel flash column chromatography to afford benzyl 2-(((2S,3R)-1-((S)-2-(5-((diethoxyphosphoryl)difluoromethyl)benzo[b]thiophene-2-carboxamido)-3,3-dimethylbutanoyl)-2-((R)-2-phenylmorpholine-4-carbonyl)pyrrolidin-3-yl)oxy)acetate. ES/MS: m/z=881.8 [M−H]⁻.

2-(((2S,3R)-1-((S)-2-(5-((diethoxyphosphoryl)difluoromethyl)benzo[b]thiophene-2-carboxamido)-3,3-dimethylbutanoyl)-2-((R)-2-phenylmorpholine-4-carbonyl)pyrrolidin-3-yl)oxy)acetic acid (C1.5). Pd/C (10% w/w, 33 mg, 0.03 mmol) was added to a solution of benzyl 2-(((2S,3R)-1-((S)-2-(5-((diethoxyphosphoryl)difluoromethyl)benzo[b]thiophene-2-carboxamido)-3,3-dimethylbutanoyl)-2-((R)-2-phenylmorpholine-4-carbonyl)pyrrolidin-3-yl)oxy)acetate (270 mg, 0.31 mmol) in EtOH (3.0 mL). The mixture was stirred under an atmosphere of hydrogen gas for 30 minutes before being filtered over Celite. Concentration of the filtrate afforded 2-(((2S,3R)-1-((S)-2-(5-((diethoxyphosphoryl)difluoromethyl)benzo[b]thiophene-2-carboxamido)-3,3-dimethylbutanoyl)-2-((R)-2-phenylmorpholine-4-carbonyl)pyrrolidin-3-yl)oxy)acetic acid. ES/MS: m/z=791.8 [M−H]⁻.

### Preparation of 3-(5-((1-(2-(((2S,3R)-1-((S)-2-amino-3,3-dimethylbutanoyl)-2-((R)-2-phenylmorpholine-4-carbonyl)pyrrolidin-3-yl)oxy)acetyl)piperidin-4-yl)methyl)-3-methyl-2-oxo-2,3-dihydro-1H-benzo[d]imidazol-1-yl)piperidine-2,6-dione (C2.2)

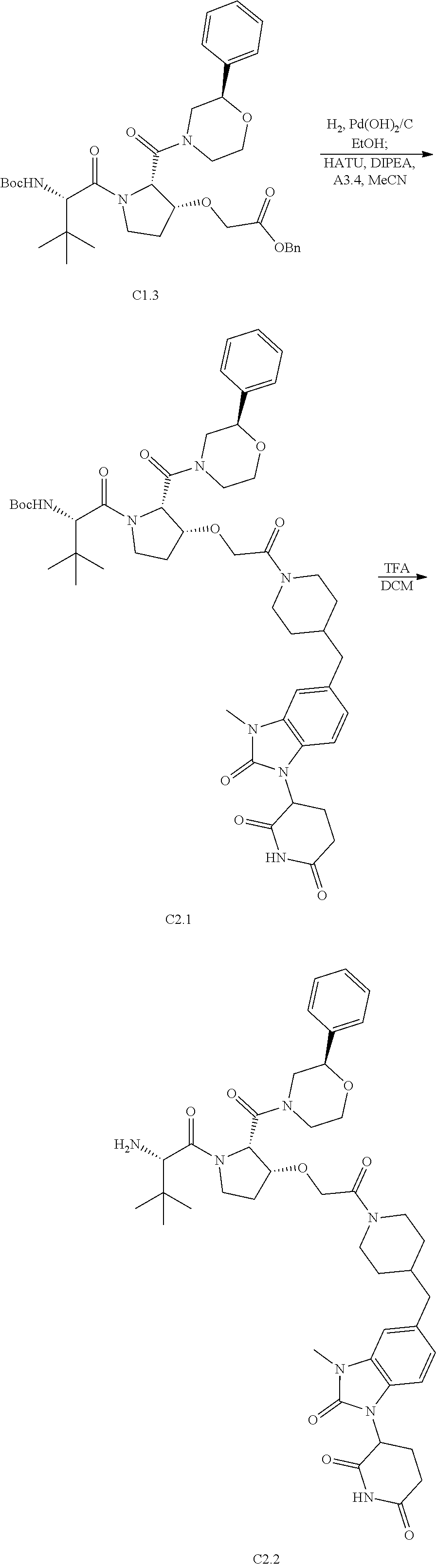

tert-butyl ((2S)-1-((2S,3R)-3-(2-(4-((1-(2,6-dioxopiperidin-3-yl)-3-methyl-2-oxo-2,3-dihydro-1H-benzo[d]imidazol-5-yl)methyl)piperidin-1-yl)-2-oxoethoxy)-2-((R)-2-phenylmorpholine-4-carbonyl)pyrrolidin-1-yl)-3,3-dimethyl-1-oxobutan-2-yl)carbamate (C2.1). Pd₂/C (10% w/w, 48 mg, 0.05 mmol) was added to a solution of benzyl 2-(((2S,3R)-1-((S)-2-((tert-butoxycarbonyl)amino)-3,3-dimethylbutanoyl)-2-((R)-2-phenylmorpholine-4-carbonyl)pyrrolidin-3-yl)oxy)acetate (290 mg, 0.46 mmol) in EtOH (3.0 mL). The mixture was stirred under an atmosphere of hydrogen gas for 30 minutes before being filtered over Celite and concentrated. The residue was suspended in MeCN (3.0 mL) and 3-(3-methyl-2-oxo-5-(piperidin-4-ylmethyl)-2,3-dihydro-1H-benzo[d]imidazol-1-yl)piperidine-2,6-dione A3.4 (200 mg, 0.46 mmol) was added followed by DIPEA (0.33 mL, 1.8 mmol) and HATU (130 mg, 0.34 mmol). After stirring for 30 minutes, the mixture was concentrated and purified by silica gel column chromatography to afford tert-butyl ((2S)-1-((2S,3R)-3-(2-(4-((1-(2,6-dioxopiperidin-3-yl)-3-methyl-2-oxo-2,3-dihydro-1H-benzo[d]imidazol-5-yl)methyl)piperidin-1-yl)-2-oxoethoxy)-2-((R)-2-phenylmorpholine-4-carbonyl)pyrrolidin-1-yl)-3,3-dimethyl-1-oxobutan-2-yl)carbamate. ES/MS: m/z=886.4 [M+H]⁺.

3-(5-((1-(2-(((2S,3R)-1-((S)-2-amino-3,3-dimethylbutanoyl)-2-((R)-2-phenylmorpholine-4-carbonyl)pyrrolidin-3-yl)oxy)acetyl)piperidin-4-yl)methyl)-3-methyl-2-oxo-2,3-dihydro-1H-benzo[d]imidazol-1-yl)piperidine-2,6-dione (C2.2). tert-butyl ((2S)-1-((2S,3R)-3-(2-(4-((1-(2,6-dioxopiperidin-3-yl)-3-methyl-2-oxo-2,3-dihydro-1H-benzo[d]imidazol-5-yl)methyl)piperidin-1-yl)-2-oxoethoxy)-2-((R)-2-phenylmorpholine-4-carbonyl)pyrrolidin-1-yl)-3,3-dimethyl-1-oxobutan-2-yl)carbamate (220 mg, 0.25 mmol) was suspended in DCM/TFA (5:1, 3 mL). After stirring for 30 min, the mixture was concentrated to afford 3-(5-((1-(2-(((2S,3R)-1-((S)-2-amino-3,3-dimethylbutanoyl)-2-((R)-2-phenylmorpholine-4-carbonyl)pyrrolidin-3-yl)oxy)acetyl)piperidin-4-yl)methyl)-3-methyl-2-oxo-2,3-dihydro-1H-benzo[d]imidazol-1-yl)piperidine-2,6-dione. ES/MS: m/z=786.4 [M+H]⁺. ¹H NMR (400 MHz, DMSO-d6) δ 6.74-6.49 (m, 5H), 6.33-6.06 (m, 3H), 4.65-4.30 (m, 2H), 3.95-3.20 (m, 9H), 3.14-2.90 (m, 4H), 2.76-2.56 (m, 4H), 2.30-1.73 (m, 8H), 1.56-1.21 (m, 3H), 1.14-0.78 (m, 3H), 0.62-0.50 (m, 2H), 0.47-0.32 (m, 9H).

### Preparation of (2S,3R)-1-((S)-2-(5-((diethoxyphosphoryl)difluoromethyl)benzo[b]thiophene-2-carboxamido)-3,3-dimethylbutanoyl)-3-(2-(4-((1-(2,6-dioxopiperidin-3-yl)-3-methyl-2-oxo-2,3-dihydro-1H-benzo[d]imidazol-5-yl)methyl)piperidin-1-yl)-2-oxoethoxy)pyrrolidine-2-carboxylate (C3.6)

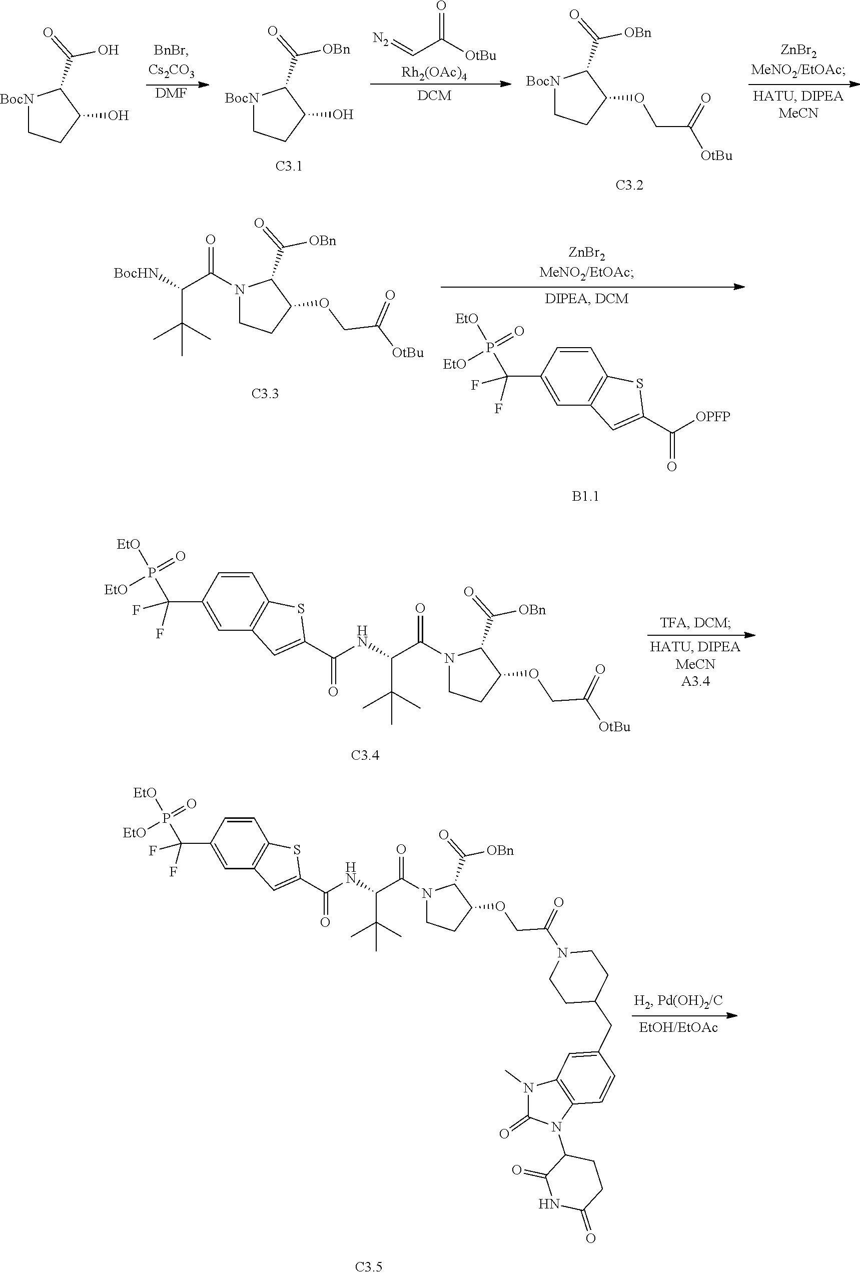

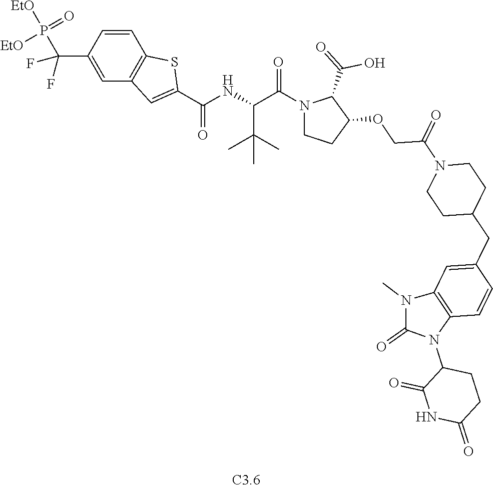 [SMILES: CCOP(=O)(OCC)C(F)(F)c1ccc2sc(C(=O)N[C@H](C(=O)N3CC[C@@H](OCC(=O)N4CCC(Cc5ccc6c(c5)n(C)c(=O)n6C5CCC(=O)NC5=O)CC4)[C@H]3C(=O)O)C(C)(C)C)cc2c1]

2-benzyl 1-(tert-butyl) (2S,3R)-3-hydroxypyrrolidine-1,2-dicarboxylate (C3.1). To a stirred solution of (2S,3R)-1-tert-butoxycarbonyl-3-hydroxy-pyrrolidine-2-carboxylic acid (700 mg, 3.0 mmol) in DMF (6.0 mL) was added Cs₂CO₃ (1.1 g, 3.3 mmol) and benzyl bromide (0.36 mL, 2.0 mmol). After stirring overnight, the mixture was diluted with EtOAc and washed with 10% Aq. LiCl then brine, dried over MgSO₄, filtered, and concentrated. Purification of the crude residue by silica gel flash column chromatography afforded 2-benzyl 1-(tert-butyl) (2S,3R)-3-hydroxypyrrolidine-1,2-dicarboxylate. ES/MS: m/z=[M+H]⁺. ¹H NMR (500 MHz, DMSO-d6) δ 7.51-7.21 (m, 5H), 5.53 (dd, J=4.9, 2.9 Hz, 1H), 5.25-5.00 (m, 2H), 4.50 (p, J=6.6 Hz, 1H), 4.26 (dd, J=9.1, 7.1 Hz, 1H), 3.44 (dtd, J=10.6, 5.4, 2.6 Hz, 1H), 3.27 (d, J=3.2 Hz, 1H), 1.84 (dd, J=14.0, 7.3 Hz, 1H), 1.39 (s, 3H), 1.26 (s, 4H).

2-benzyl 1-(tert-butyl) (2S,3R)-3-(2-(tert-butoxy)-2-oxoethoxy)pyrrolidine-1,2-dicarboxylate (C3.2). Tert-butyl diazoacetate (0.39 mL, 2.8 mmol) was added to a mixture of 2-benzyl 1-(tert-butyl) (2S,3R)-3-hydroxypyrrolidine-1,2-dicarboxylate (450 mg, 1.4 mmol) and Rh₂(OAc)₄ (62 mg, 0.14 mmol) in DCM (14 mL) at 0° C. The mixture was warmed to room temperature and stirred for 5 hours. The mixture was diluted with EtOAc and washed with water and brine, dried over MgSO₄, filtered, and concentrated. Purification of the residue by silica gel flash column chromatography afforded benzyl (2S,3R)-3-(2-(tert-butoxy)-2-oxoethoxy)-1-((S)-2-((tert-butoxycarbonyl)amino)-3,3-dimethylbutanoyl)pyrrolidine-2-carboxylate. ES/MS: m/z=457.9 [M+Na]⁺. ¹H NMR (400 MHz, Chloroform-d) δ 7.43-7.30 (m, 5H), 5.33-5.09 (m, 2H), 4.59 (d, J=7.3 Hz, 0.5H, rotamer), 4.49 (d, J=7.3 Hz, 0.5H, rotamer), 4.41-4.27 (m, 1H), 3.96 (d, J=3.0 Hz, 1H), 3.91 (d, J=2.9 Hz, 1H), 3.75-3.56 (m, 1H), 3.41-3.26 (m, 1H), 2.24-2.09 (m, 2H), 1.55 (s, 4.5H, rotamer), 1.46 (s, 4.5H, rotamer), 1.44 (s, 4.5H, rotamer), 1.33 (s, 4.5H, rotamer).

benzyl (2S,3R)-3-(2-(tert-butoxy)-2-oxoethoxy)-1-((S)-2-((tert-butoxycarbonyl)amino)-3,3-dimethylbutanoyl)pyrrolidine-2-carboxylate (C3.3). Zinc Bromide (600 mg, 2.6 mmol) was added to a solution of benzyl (2S,3R)-3-(2-(tert-butoxy)-2-oxoethoxy)-1-((S)-2-((tert-butoxycarbonyl)amino)-3,3-dimethylbutanoyl)pyrrolidine-2-carboxylate (230 mg, 0.53 mmol) in MeNO₂/EtOAc (5:1, 6.0 mL) at 0° C. After warming to room temperature and stirring for 4 hours, the mixture was diluted with EtOAc, washed with sat. aq. NaHCO₃, dried over MgSO₄, filtered, and concentrated. The resulting residue was resuspended in MeCN (6.0 mL) and (2S)-2-(tert-butoxycarbonylamino)-3,3-dimethyl-butanoic acid (130 mg, 0.58 mmol), DIPEA (0.19 mL, 1.1 mmol) and HATU (150 mg, 0.39 mmol) were added. After stirring for 30 minutes, the mixture was concentrated and purified by silica gel flash column chromatography to afford benzyl (2S,3R)-3-(2-(tert-butoxy)-2-oxoethoxy)-1-((S)-2-(5-((diethoxyphosphoryl)difluoromethyl)benzo[b]thiophene-2-carboxamido)-3,3-dimethylbutanoyl)pyrrolidine-2-carboxylate. ES/MS: m/z=548.9 [M+H]⁺.

benzyl (2S,3R)-3-(2-(tert-butoxy)-2-oxoethoxy)-1-((S)-2-(5-((diethoxyphosphoryl)difluoromethyl)benzo[b]thiophene-2-carboxamido)-3,3-dimethylbutanoyl)pyrrolidine-2-carboxylate (C3.4). Zinc Bromide (310 mg, 1.4 mmol) was added to a solution of benzyl (2S,3R)-3-(2-(tert-butoxy)-2-oxoethoxy)-1-((S)-2-(5-((diethoxyphosphoryl)difluoromethyl)benzo[b]thiophene-2-carboxamido)-3,3-dimethylbutanoyl)pyrrolidine-2-carboxylate (150 mg, 0.27 mmol) in MeNO₂/EtOAc (5:1, 3.0 mL) at 0° C. After warming to room temperature and stirring for 4 hours, the mixture was diluted with EtOAc, washed with sat. aq. NaHCO₃, dried over MgSO₄, filtered, and concentrated. The resulting residue was resuspended in DCM (2.0 mL) and (2,3,4,5,6-pentafluorophenyl) 5-[diethoxyphosphoryl(difluoro)methyl]benzothiophene-2-carboxylate B1.1 (150 mg, 0.27 mmol) and DIPEA (0.15 mL, 0.8 mmol) were added. After stirring overnight, the mixture was concentrated and purified by silica gel flash column chromatography to afford benzyl (2S,3R)-1-((S)-2-(5-((diethoxyphosphoryl)difluoromethyl)benzo[b]thiophene-2-carboxamido)-3,3-dimethylbutanoyl)-3-(2-(4-((1-(2,6-dioxopiperidin-3-yl)-3-methyl-2-oxo-2,3-dihydro-1H-benzo[d]imidazol-5-yl)methyl)piperidin-1-yl)-2-oxoethoxy)pyrrolidine-2-carboxylate. ES/MS: m/z=792.8 [M−H]—. ¹H NMR (400 MHz, Methanol-d4) δ 8.22-8.18 (m, 1H), 8.15 (s, 1H), 8.08 (d, J=8.6 Hz, 1H), 7.67-7.61 (m, 1H), 7.45-7.28 (m, 5H), 5.22-5.13 (m, 2H), 4.77 (d, J=6.4 Hz, 1H), 4.47-4.40 (m, 1H), 4.29-4.15 (m, 4H), 3.96 (d, J=3.5 Hz, 1H), 2.34-2.07 (m, 2H), 1.46 (s, 9H), 1.13-1.06 (m, 9H).

benzyl (2S,3R)-1-((S)-2-(5-((diethoxyphosphoryl)difluoromethyl)benzo[b]thiophene-2-carboxamido)-3,3-dimethylbutanoyl)-3-(2-(4-((1-(2,6-dioxopiperidin-3-yl)-3-methyl-2-oxo-2,3-dihydro-1H-benzo[d]imidazol-5-yl)methyl)piperidin-1-yl)-2-oxoethoxy)pyrrolidine-2-carboxylate (C3.5). benzyl (2S,3R)-1-((S)-2-(5-((diethoxyphosphoryl)difluoromethyl)benzo[b]thiophene-2-carboxamido)-3,3-dimethylbutanoyl)-3-(2-(4-((1-(2,6-dioxopiperidin-3-yl)-3-methyl-2-oxo-2,3-dihydro-1H-benzo[d]imidazol-5-yl)methyl)piperidin-1-yl)-2-oxoethoxy)pyrrolidine-2-carboxylate (100 mg, 0.13 mmol) was suspended in DCM/TFA (4:1, 2.5 mL). After stirring for 45 minutes, the mixture was concentrated and resuspended in MeCN (2.5 mL). 3-(3-methyl-2-oxo-5-(piperidin-4-ylmethyl)-2,3-dihydro-1H-benzo[d]imidazol-1-yl)piperidine-2,6-dione A3.4 (62 mg, 0.14 mmol) was added followed by DIPEA (0.24 mL, 1.4 mmol) and HATU (48 mg, 0.13 mmol). After stirring for 30 minutes, the mixture was concentrated and purified by silica gel column chromatography to afford benzyl (2S,3R)-1-((S)-2-(5-((diethoxyphosphoryl)difluoromethyl)benzo[b]thiophene-2-carboxamido)-3,3-dimethylbutanoyl)-3-(2-(4-((1-(2,6-dioxopiperidin-3-yl)-3-methyl-2-oxo-2,3-dihydro-1H-benzo[d]imidazol-5-yl)methyl)piperidin-1-yl)-2-oxoethoxy)pyrrolidine-2-carboxylate. ES/MS: m/z=1074.8 [M−H]—.

(2S,3R)-1-((S)-2-(5-((diethoxyphosphoryl)difluoromethyl)benzo[b]thiophene-2-carboxamido)-3,3-dimethylbutanoyl)-3-(2-(4-((1-(2,6-dioxopiperidin-3-yl)-3-methyl-2-oxo-2,3-dihydro-1H-benzo[d]imidazol-5-yl)methyl)piperidin-1-yl)-2-oxoethoxy)pyrrolidine-2-carboxylic acid (C3.6). Pd(OH)₂/C (20% w/w, 17 mg, 0.02 mmol) was added to a solution of benzyl (2S,3R)-1-((S)-2-(5-((diethoxyphosphoryl)difluoromethyl)benzo[b]thiophene-2-carboxamido)-3,3-dimethylbutanoyl)-3-(2-(4-((1-(2,6-dioxopiperidin-3-yl)-3-methyl-2-oxo-2,3-dihydro-1H-benzo[d]imidazol-5-yl)methyl)piperidin-1-yl)-2-oxoethoxy)pyrrolidine-2-carboxylate (130 mg, 0.12 mmol) in EtOH/EtOAc (2:1, 6.0 mL). The mixture was stirred under an atmosphere of hydrogen gas for 3 hours, then filtered over Celite and concentrated to afford (2S,3R)-1-((S)-2-(5-((diethoxyphosphoryl)difluoromethyl)benzo[b]thiophene-2-carboxamido)-3,3-dimethylbutanoyl)-3-(2-(4-((1-(2,6-dioxopiperidin-3-yl)-3-methyl-2-oxo-2,3-dihydro-1H-benzo[d]imidazol-5-yl)methyl)piperidin-1-yl)-2-oxoethoxy)pyrrolidine-2-carboxylic acid. ES/MS: m/z=984.8 [M−H]⁻. ¹H NMR (400 MHz, DMSO-d) 11.09 (s, 1H), 8.57-8.48 (m, 2H), 8.21 (d, J=8.5 Hz, 1H), 8.11 (s, 1H), 7.60 (d, J=8.7 Hz, 1H), 7.04-6.98 (m, 2H), 6.84 (d, J=8.1 Hz, 2H), 5.35 (dd, J=12.7, 5.4 Hz, 2H), 4.76 (d, J=8.8 Hz, 2H), 4.60-4.46 (m, 1H), 4.39-4.07 (m, 7H), 3.86 (s, 1H), 3.76-3.68 (m, 2H), 3.33 (s, 3H), 2.88 (dd, J=12.4, 4.8 Hz, 2H), 2.82-2.64 (m, 1H), 2.16-1.95 (m, 3H), 1.76 (s, 1H), 1.60 (d, J=12.6 Hz, 2H), 1.23 (td, J=7.0, 3.2 Hz, 5H), 1.10-0.98 (m, 9H).

### Preparation of (2S,3R)-1-((benzyloxy)carbonyl)-3-((1-(tert-butoxycarbonyl)piperidin-4-yl)methyl)pyrrolidine-2-carboxylic acid (C4.3)

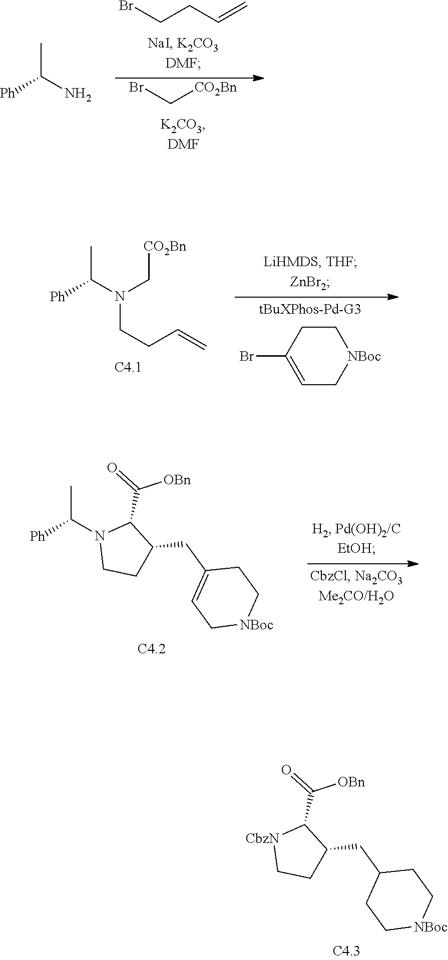

benzyl (S)—N-(but-3-en-1-yl)-N-(1-phenylethyl)glycinate (C4.1). A mixture of (1S)-1-phenylethanamine (5.5 mL, 43 mmol), 4-bromobut-1-ene (3.4 mL, 33 mmol), K₂CO₃ (18.4 g, 133 mmol), and NaI (20.0 g, 133 mmol) was heated in DMF (45 mL) overnight at 90° C. After cooling to room temperature, the mixture was diluted with EtOAc and washed with water and brine. The organic layer was dried over MgSO4, filtered, and concentrated. The resulting residue was suspended in DMF (30 mL) and K₂CO₃ (9.2 g, 67 mmol) was added followed by benzyl bromoacetate (5.3 mL, 33 mmol). The resulting mixture was stirred overnight at 50° C. then cooled to room temperature. The mixture was diluted with EtOAc and washed with water and brine. The organic layer was dried over MgSO4, filtered, and concentrated. Purification by silica gel flash column chromatography afforded benzyl (S)—N-(but-3-en-1-yl)-N-(1-phenylethyl)glycinate. ES/MS: m/z=324.0 [M+H]⁺.

tert-butyl 4-(((2S,3R)-2-((benzyloxy)carbonyl)-1-((S)-1-phenylethyl)pyrrolidin-3-yl)methyl)-3,6-dihydropyridine-1(2H)-carboxylate (C4.2). LiHMDS (1.0 M in THF, 5.6 mL, 5.6 mmol) was added to a solution of benzyl (S)—N-(but-3-en-1-yl)-N-(1-phenylethyl)glycinate (1.5 g, 4.6 mmol) in THF (15 mL) at −78° C. After stirring for 10 minutes, the mixture was allowed to warm to 0° C. and stir for 30 minutes before being cooled to −78° C. ZnBr₂ (3.1 g, 14 mmol) in THF (12 mL) was then added slowly. The mixture was warmed to room temperature and allowed to stir for 1 hour. tert-butyl 4-bromo-3,6-dihydro-2H-pyridine-1-carboxylate (1.1 g, 4.3 mmol) and tBu-xPhos-Pd-G3 (320 mg, 0.46 mmol) were then added. The mixture was heated to 30° C. and allowed to stir overnight. The mixture was diluted with EtOAc and washed with 1N HCl and sat. aq. NaHCO₃, then dried over MgSO₄, filtered, and concentrated. Purification by silica gel flash column chromatography afforded tert-butyl 4-(((2S,3R)-2-((benzyloxy)carbonyl)-1-((S)-1-phenylethyl)pyrrolidin-3-yl)methyl)-3,6-dihydropyridine-1(2H)-carboxylate. ES/MS: m/z=505.0 [M+H]⁺. ¹H NMR (400 MHz, Methanol-d4) δ 7.44-7.13 (m, 1 OH), 5.30-5.19 (m, 1H), 5.11-5.00 (m, 2H), 3.77-3.60 (m, 3H), 3.43-3.34 (m, 3H), 3.06-2.84 (m, 2H), 2.61-2.47 (m, 1H), 2.01-1.86 (m, 3H), 1.80-1.52 (m, 2H), 1.46-1.41 (m, 9H), 1.30 (d, J=6.7 Hz, 3H).

(2S,3R)-1-((benzyloxy)carbonyl)-3-((1-(tert-butoxycarbonyl)piperidin-4-yl)methyl)pyrrolidine-2-carboxylic acid (C4.3). A mixture of tert-butyl 4-(((2S,3R)-2-((benzyloxy)carbonyl)-1-((S)-1-phenylethyl)pyrrolidin-3-yl)methyl)-3,6-dihydropyridine-1(2H)-carboxylate (1.4 g, 2.9 mmol) and Pd(OH)₂/C (20% w/w, 2.0 g, 2.9 mmol) in EtOH (30 mL) were stirred under an atmosphere of hydrogen gas overnight. The mixture was filtered over celite and concentrated. The resulting residue was suspended in acetone/H₂O (1:1, 30 mL) and Na₂CO₃ (450 mg, 4.3 mmol) was added followed by benzyl chloroformate (0.45 mL, 3.1 mmol) at 0° C. The mixture was allowed to warm to room temperature and stir overnight. The mixture was diluted with EtOAc and washed with 1N HCl. The aqueous layer was washed with two portions of EtOAc, and the combined organic layers were dried over MgSO₄, filtered, and concentrated to afford crude (2S,3R)-1-((benzyloxy)carbonyl)-3-((1-(tert-butoxycarbonyl)piperidin-4-yl)methyl)pyrrolidine-2-carboxylic acid, which was used without further purification. ES/MS: m/z=445.0 [M−H]⁻.

### Preparation of diethyl ((2-(((S)-3,3-dimethyl-1-oxo-1-((2S,3R)-2-((R)-2-phenylmorpholine-4-carbonyl)-3-(piperidin-4-ylmethyl)pyrrolidin-1-yl)butan-2-yl)carbamoyl)benzo[b]thiophen-5-yl)difluoromethyl)phosphonate (C4.7)

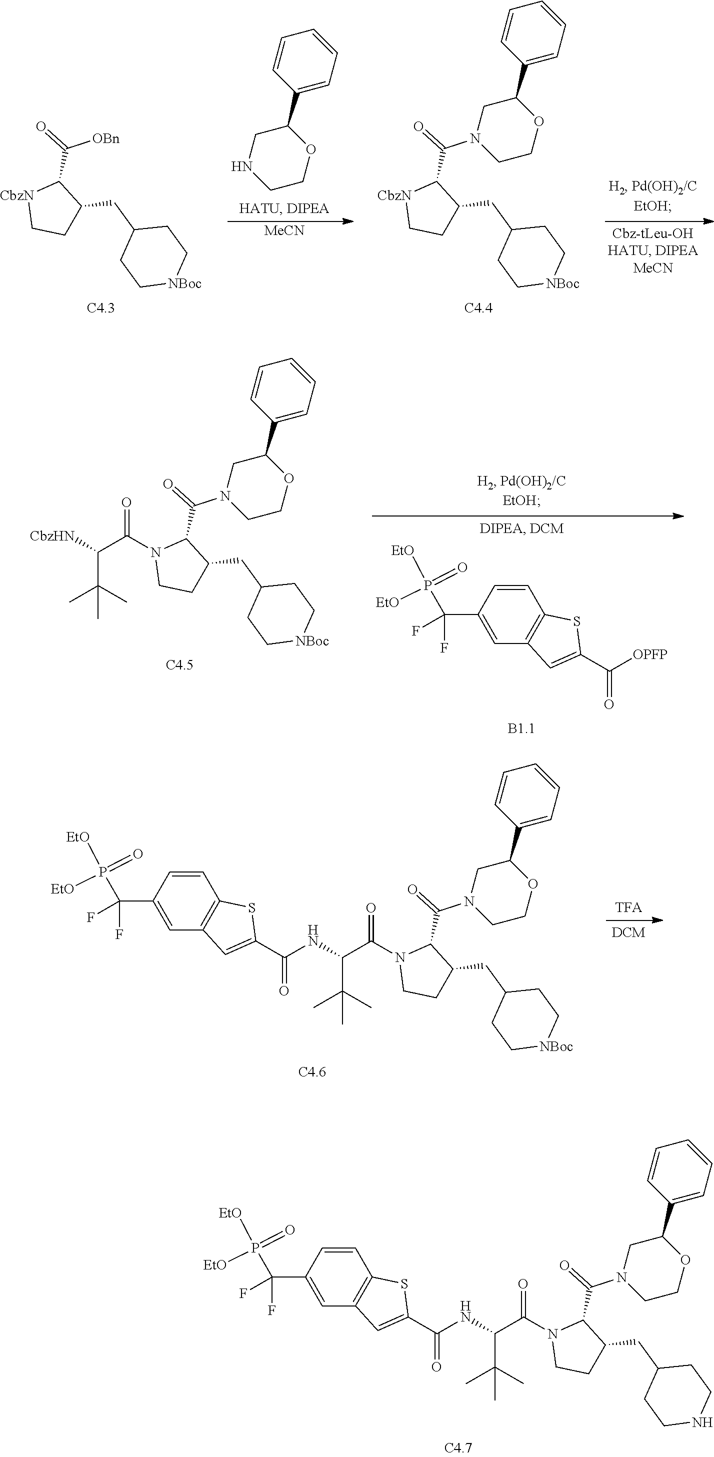

Tert-butyl 4-(((2S,3R)-1-((benzyloxy)carbonyl)-2-((R)-2-phenylmorpholine-4-carbonyl)pyrrolidin-3-yl)methyl)piperidine-1-carboxylate (C4.4). To the crude residue from the previous step (1.2 g, 2.9 mmol) in MeCN (20 mL) was added (2R)-2-phenylmorpholine (420 mg, 2.6 mmol), DIPEA (1.0 mL, 5.7 mmol), and HATU (810 mg, 2.1 mmol). After stirring for 30 minutes, the mixture was concentrated and purified by silica gel flash column chromatography to afford tert-butyl 4-(((2S,3R)-1-((benzyloxy)carbonyl)-2-((R)-2-phenylmorpholine-4-carbonyl)pyrrolidin-3-yl)methyl)piperidine-1-carboxylate. ES/MS: m/z=535.9 [M-tBu+H]⁺. ¹H NMR (400 MHz, Chloroform-d) δ 7.53-7.05 (m, 10H), 5.26-0.90 (m, 35H).

tert-butyl 4-(((2S,3R)-1-((S)-2-(((benzyloxy)carbonyl)amino)-3,3-dimethylbutanoyl)-2-((R)-2-phenylmorpholine-4-carbonyl)pyrrolidin-3-yl)methyl)piperidine-1-carboxylate (C4.5). A mixture of tert-butyl 4-(((2S,3R)-1-((benzyloxy)carbonyl)-2-((R)-2-phenylmorpholine-4-carbonyl)pyrrolidin-3-yl)methyl)piperidine-1-carboxylate (1.1 g, 1.9 mmol) and Pd(OH)₂/C (20% w/w, 260 mg, 0.37 mmol) in EtOH (18 mL) was stirred under an atmosphere of hydrogen gas for 1 hour. The mixture was filtered over celite and concentrated, then resuspended in MeCN (15 mL). (2S)-2-(benzyloxycarbonylamino)-3,3-dimethyl-butanoic acid (590 mg, 2.2 mmol), DIPEA (0.66 mL, 3.7 mmol) and HATU (660 mg, 1.7 mmol) were then added. After stirring for 30 minutes, the mixture was concentrated and purified by silica gel flash column chromatography to afford tert-butyl 4-(((2S,3R)-1-((S)-2-(((benzyloxy)carbonyl)amino)-3,3-dimethylbutanoyl)-2-((R)-2-phenylmorpholine-4-carbonyl)pyrrolidin-3-yl)methyl)piperidine-1-carboxylate. ES/MS: m/z=649.0 [M-tBu+H]⁺. ¹H NMR (400 MHz, Methanol-d) δ 7.56-7.22 (m, 10H), 5.32-1.11 (m, 36H), 1.09-0.97 (m, 9H).

tert-butyl 4-(((2S,3R)-1-((S)-2-(5-((diethoxyphosphoryl)difluoromethyl)benzo[b]thiophene-2-carboxamido)-3,3-dimethylbutanoyl)-2-((R)-2-phenylmorpholine-4-carbonyl)pyrrolidin-3-yl)methyl)piperidine-1-carboxylate (C4.6). A mixture of tert-butyl 4-(((2S,3R)-1-((S)-2-(((benzyloxy)carbonyl)amino)-3,3-dimethylbutanoyl)-2-((R)-2-phenylmorpholine-4-carbonyl)pyrrolidin-3-yl)methyl)piperidine-1-carboxylate (410 mg, 0.58 mmol) and Pd(OH)₂/C (100 mg, 0.15 mmol) in EtOH (5 mL) was stirred under an atmosphere of hydrogen gas for 1 hour. The mixture was then filtered over celite and concentrated before being resuspended in DCM (5 mL). DIPEA (0.3 mL, 1.8 mmol) and (2,3,4,5,6-pentafluorophenyl) 5-[diethoxyphosphoryl(difluoro)methyl]benzothiophene-2-carboxylate B1.1 (310 mg, 0.58 mmol) were then added. The mixture was allowed to stir overnight before being concentrated. Purification by silica gel flash column chromatography afforded tert-butyl 4-(((2S,3R)-1-((S)-2-(5-((diethoxyphosphoryl)difluoromethyl)benzo[b]thiophene-2-carboxamido)-3,3-dimethylbutanoyl)-2-((R)-2-phenylmorpholine-4-carbonyl)pyrrolidin-3-yl)methyl)piperidine-1-carboxylate. ES/MS: m/z=457.8 [proline-tLeu fragmentation]⁺.

diethyl ((2-(((S)-3,3-dimethyl-1-oxo-1-((2S,3R)-2-((R)-2-phenylmorpholine-4-carbonyl)-3-(piperidin-4-ylmethyl)pyrrolidin-1-yl)butan-2-yl)carbamoyl)benzo[b]thiophen-5-yl)difluoromethyl)phosphonate (C4.7). Tert-butyl 4-(((2S,3R)-1-((S)-2-(5-((diethoxyphosphoryl)difluoromethyl)benzo[b]thiophene-2-carboxamido)-3,3-dimethylbutanoyl)-2-((R)-2-phenylmorpholine-4-carbonyl)pyrrolidin-3-yl)methyl)piperidine-1-carboxylate (450 mg, 0.34 mmol) was suspended in DCM/TFA (5:1, 5 mL). After stirring for 30 minutes, the mixture was concentrated, resuspended in EtOAc, and washed with sat. aq. NaHCO₃. The organic layer was concentrated to afford diethyl ((2-(((S)-3,3-dimethyl-1-oxo-1-((2S,3R)-2-((R)-2-phenylmorpholine-4-carbonyl)-3-(piperidin-4-ylmethyl)pyrrolidin-1-yl)butan-2-yl)carbamoyl)benzo[b]thiophen-5-yl)difluoromethyl)phosphonate. ES/MS: m/z=817.4 [M+H]⁺. ¹H NMR 1H NMR (400 MHz, DMSO-d6) δ 8.63-8.42 (m, 2H), 8.25 (d, J=8.5 Hz, 1H), 8.18-8.12 (m, 1H), 7.64 (d, J=8.5 Hz, 1H), 7.53-7.29 (m, 4H), 5.28-4.67 (m, 3H), 4.52-4.34 (m, 2H), 4.26-0.99 (m, 39H).

### Preparation of (2S,3R)-1-((benzyloxy)carbonyl)-3-(2-(1-(tert-butoxycarbonyl)piperidin-4-yl)ethyl)pyrrolidine-2-carboxylic acid (C4.8)

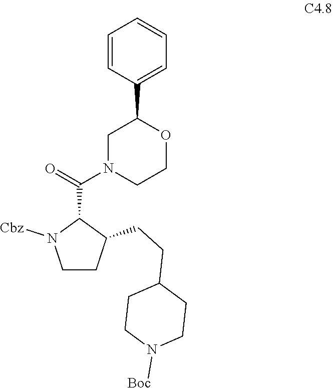 [SMILES: CC(C)(C)OC(=O)N1CCC(CC[C@@H]2CCN(C(=O)OCc3ccccc3)[C@@H]2C(=O)N2CCO[C@H](c3ccccc3)C2)CC1]

(2S,3R)-1-((benzyloxy)carbonyl)-3-(2-(1-(tert-butoxycarbonyl)piperidin-4-yl)ethyl)pyrrolidine-2-carboxylic acid (C4.8). C4.8 was prepared analogously to C4.4, using tert-butyl 4-(bromomethylene)piperidine-1-carboxylate instead of tert-butyl 4-bromo-3,6-dihydropyridine-1(2H)-carboxylate. ES/MS: m/z=606.4 [M+H]⁺.

### Preparation of 3-(5-((4-(((2S,3R)-1-((S)-2-amino-3,3-dimethylbutanoyl)-2-((R)-2-phenylmorpholine-4-carbonyl)pyrrolidin-3-yl)methyl)piperidin-1-yl)methyl)-3-methyl-2-oxo-2,3-dihydro-1H-benzo[d]imidazol-1-yl)piperidine-2,6-dione (C5.3)

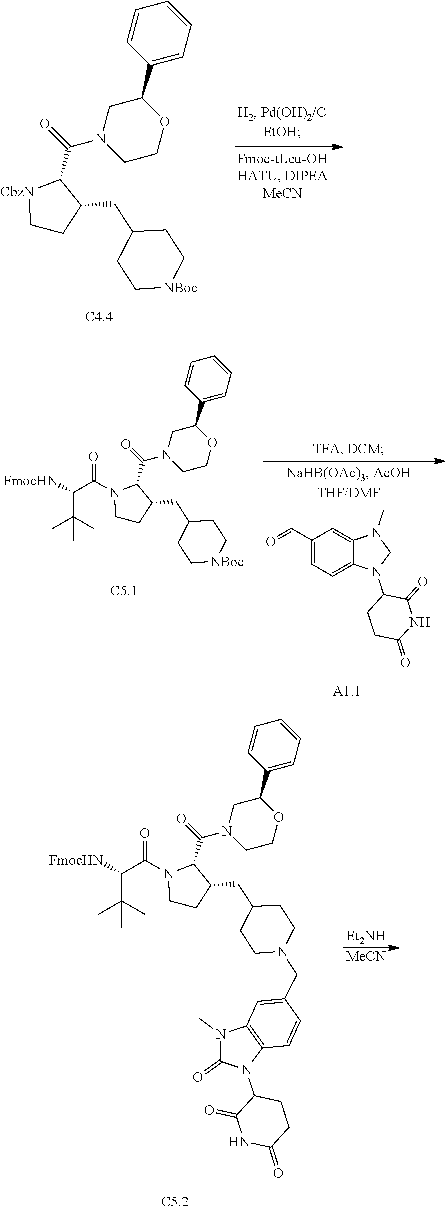

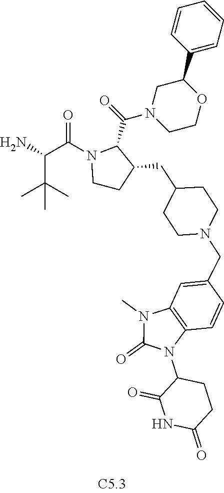 [SMILES: Cn1c(=O)n(C2CCC(=O)NC2=O)c2ccc(CN3CCC(C[C@@H]4CCN(C(=O)[C@@H](N)C(C)(C)C)[C@@H]4C(=O)N4CCO[C@H](c5ccccc5)C4)CC3)cc21]

tert-butyl 4-(((2S,3R)-1-((S)-2-((((9H-fluoren-9-yl)methoxy)carbonyl)amino)-3,3-dimethylbutanoyl)-2-((R)-2-phenylmorpholine-4-carbonyl)pyrrolidin-3-yl)methyl)piperidine-1-carboxylate (C5.1). A mixture of tert-butyl 4-(((2S,3R)-1-((benzyloxy)carbonyl)-2-((R)-2-phenylmorpholine-4-carbonyl)pyrrolidin-3-yl)methyl)piperidine-1-carboxylate (850 mg, 1.44 mmol) and Pd(OH)₂/C (20% w/w, 300 mg, 0.43 mmol) in EtOH (20 mL) was stirred under an atmosphere of hydrogen gas for 1 hour. The mixture was filtered over celite and concentrated, then resuspended in MeCN (20 mL). (2S)-2-(9H-fluoren-9-ylmethoxycarbonylamino)-3,3-dimethyl-butanoic acid (610 mg, 1.72 mmol) was added followed by DIPEA (0.38 mL, 2.15 mmol) and HATU (710 mg, 1.87 mmol). After stirring for 1 hour, the mixture was concentrated and purified by silica gel flash column chromatography to afford tert-butyl 4-(((2S,3R)-1-((S)-2-((((9H-fluoren-9-yl)methoxy)carbonyl)amino)-3,3-dimethylbutanoyl)-2-((R)-2-phenylmorpholine-4-carbonyl)pyrrolidin-3-yl)methyl)piperidine-1-carboxylate. ES/MS: m/z=694.1 [M-Boc+H]⁺. ¹H NMR 1H NMR (400 MHz, Methanol-d4) δ 7.80 (d, J=7.6 Hz, 2H), 7.66 (t, J=7.2 Hz, 2H), 7.51-7.20 (m, 9H), 5.20-4.92 (m, 1H), 4.78-4.59 (m, 1H), 4.48-4.16 (m, 6H), 4.12-3.86 (m, 5H), 3.85-3.40 (m, 3H), 3.26-3.05 (m, 1H), 3.02-2.37 (m, 5H), 2.18-1.97 (m, 3H), 1.91-1.51 (m, 4H), 1.48-1.40 (m, 9H), 1.10-0.92 (m, 9H).

(9H-fluoren-9-yl)methyl ((2S)-1-((2S,3R)-3-((1-((1-(2,6-dioxopiperidin-3-yl)-3-methyl-2-oxo-2,3-dihydro-1H-benzo[d]imidazol-5-yl)methyl)piperidin-4-yl)methyl)-2-((R)-2-phenylmorpholine-4-carbonyl)pyrrolidin-1-yl)-3,3-dimethyl-1-oxobutan-2-yl)carbamate (C5.2). tert-butyl 4-(((2S,3R)-1-((S)-2-((((9H-fluoren-9-yl)methoxy)carbonyl)amino)-3,3-dimethylbutanoyl)-2-((R)-2-phenylmorpholine-4-carbonyl)pyrrolidin-3-yl)methyl)piperidine-1-carboxylate (1.05 g, 1.3 mmol) was suspended in DCM/TFA (5:1, 12 mL). After stirring for 1 hour, the mixture was concentrated. The residue was suspended in EtOAc and washed with sat. aq. NaHCO₃, dried over MgSO4, filtered, and concentrated. The resulting residue was suspended in THF/DMF (3:1, 30 mL) and 1-(2,6-dioxo-3-piperidyl)-3-methyl-2-oxo-benzimidazole-5-carbaldehyde (650 mg, 2.3 mmol) was added followed by AcOH (0.23 mL, 3.97 mmol). After stirring for 40 minutes, sodium triacetoxyborohydride (1.26 g, 5.96 mmol) was added. After stirring overnight, the mixture was diluted with EtOAc and washed with sat. aq. NaHCO₃. The organic layer was dried over MgSO4, filtered, and concentrated. Purification by C18 chromatography afforded (9H-fluoren-9-yl)methyl ((2S)-1-((2S,3R)-3-((1-((1-(2,6-dioxopiperidin-3-yl)-3-methyl-2-oxo-2,3-dihydro-1H-benzo[d]imidazol-5-yl)methyl)piperidin-4-yl)methyl)-2-((R)-2-phenylmorpholine-4-carbonyl)pyrrolidin-1-yl)-3,3-dimethyl-1-oxobutan-2-yl)carbamate. ES/MS: m/z=964.4 [M+H]⁺. ¹H NMR (400 MHz, Methanol-d4) δ 7.88-7.18 (m, 16H), 5.44-5.09 (m, 2H), 5.08-0.92 (m, 45H).

3-(5-((4-(((2S,3R)-1-((S)-2-amino-3,3-dimethylbutanoyl)-2-((R)-2-phenylmorpholine-4-carbonyl)pyrrolidin-3-yl)methyl)piperidin-1-yl)methyl)-3-methyl-2-oxo-2,3-dihydro-1H-benzo[d]imidazol-1-yl)piperidine-2,6-dione (C5.3). To a solution of (9H-fluoren-9-yl)methyl ((2S)-1-((2S,3R)-3-((1-((1-(2,6-dioxopiperidin-3-yl)-3-methyl-2-oxo-2,3-dihydro-1H-benzo[d]imidazol-5-yl)methyl)piperidin-4-yl)methyl)-2-((R)-2-phenylmorpholine-4-carbonyl)pyrrolidin-1-yl)-3,3-dimethyl-1-oxobutan-2-yl)carbamate (250 mg, 0.26 mmol) in acetonitrile (5 mL) was added diethylamine (0.5 mL). After stirring for 1 hour, the mixture was concentrated. Purification by C18 chromatography afforded 3-(5-((4-(((2S,3R)-1-((S)-2-amino-3,3-dimethylbutanoyl)-2-((R)-2-phenylmorpholine-4-carbonyl)pyrrolidin-3-yl)methyl)piperidin-1-yl)methyl)-3-methyl-2-oxo-2,3-dihydro-1H-benzo[d]imidazol-1-yl)piperidine-2,6-dione (180 mg, 94%). ES/MS: m/z=742.4 [M+H]⁺. ¹H NMR (400 MHz, Methanol-d4) δ 7.53-7.47 (m, 1H), 7.45-7.17 (m, 7H), 5.44-5.35 (m, 1H), 5.27-1.07 (m, 43H).

### Preparation of 3-(5-((4-(2-((2S,3R)-1-((S)-2-amino-3,3-dimethylbutanoyl)-2-((R)-2-phenylmorpholine-4-carbonyl)pyrrolidin-3-yl)ethyl)piperidin-1-yl)methyl)-3-methyl-2-oxo-2,3-dihydro-1H-benzo[d]imidazol-1-yl)piperidine-2,6-dione (C5.4)

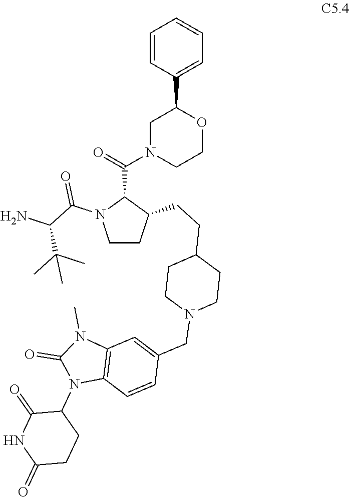 [SMILES: Cn1c(=O)n(C2CCC(=O)NC2=O)c2ccc(CN3CCC(CC[C@@H]4CCN(C(=O)[C@@H](N)C(C)(C)C)[C@@H]4C(=O)N4CCO[C@H](c5ccccc5)C4)CC3)cc21]

3-(5-((4-(2-((2S,3R)-1-((S)-2-amino-3,3-dimethylbutanoyl)-2-((R)-2-phenylmorpholine-4-carbonyl)pyrrolidin-3-yl)ethyl)piperidin-1-yl)methyl)-3-methyl-2-oxo-2,3-dihydro-1H-benzo[d]imidazol-1-yl)piperidine-2,6-dione (C5.4). C5.4 was prepared analogously to C5.5 using C4.8 instead of C4.4. ES/MS: m/z=756.4 [M+H]⁺.

### Preparation of (2S,3R)-1-((S)-2-amino-3,3-dimethylbutanoyl)-2-((R)-2-phenylmorpholine-4-carbonyl)pyrrolidin-3-yl 4-((1-(2,6-dioxopiperidin-3-yl)-3-methyl-2-oxo-2,3-dihydro-1H-benzo[d]imidazol-5-yl)methyl)piperidine-1-carboxylate (C6.5)

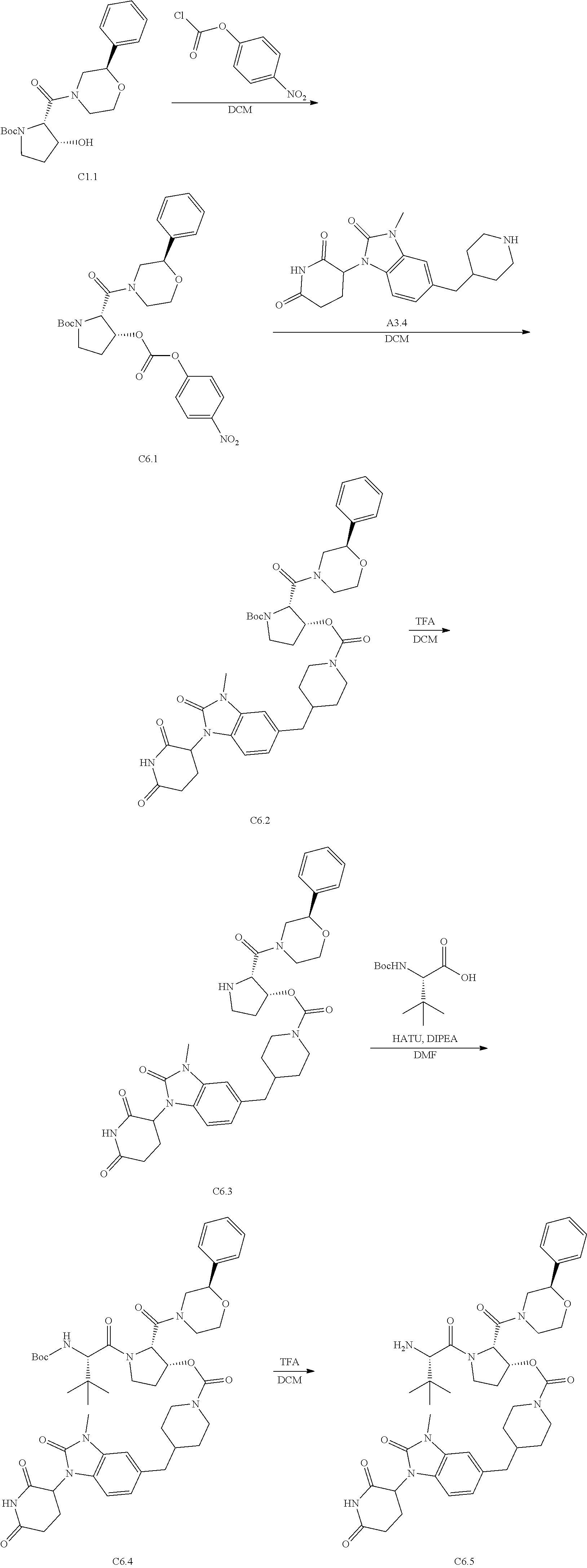

tert-butyl (2S,3R)-3-(((4-nitrophenoxy)carbonyl)oxy)-2-((R)-2-phenylmorpholine-4-carbonyl)pyrrolidine-1-carboxylate (C6.1). 4-Nitrophenylchloroformate (120 mg, 0.632 mmol) was added to a solution of tert-butyl (2S,3R)-3-hydroxy-2-((R)-2-phenylmorpholine-4-carbonyl)pyrrolidine-1-carboxylate (200 mg, 0.531 mmol) and pyridine (50 mg, 0.632 mmol) in CH₂Cl₂ (3 ml). The reaction mixture was stirred overnight. The mixture was washed with three portions of NaHSO₄ 1M and two portions of brine; dried (MgSO₄) and concentrated in vacuo to give tert-butyl (2S,3R)-3-(((4-nitrophenoxy)carbonyl)oxy)-2-((R)-2-phenylmorpholine-4-carbonyl)pyrrolidine-1-carboxylate (C6.1). ES/MS: m/z=564 [M+Na]⁺.

(2S,3R)-1-(tert-butoxycarbonyl)-2-((R)-2-phenylmorpholine-4-carbonyl)pyrrolidin-3-yl 4-((1-(2,6-dioxopiperidin-3-yl)-3-methyl-2-oxo-2,3-dihydro-1H-benzo[d]imidazol-5-yl)methyl)piperidine-1-carboxylate (C6.2). A3.4 (50 mg, 0.14 mmol) was added to a solution of C6.1 (70 mg, 0.13 mmol) in DCM and the mixture was stirred at room temperature for 3 hours. The mixture was then washed with three portions of aqueous 1M NaHSO₄, three portions of saturated aqueous NaHCO₃ and two portions of brine. The organic layer was dried over MgSO₄ and concentrated in vacuo. The residue was purified by automated silica gel chromatography (EtOAc/hexanes, 0 to 100%) to give C6.2. ES/MS: m/z=759.1 [M+H]⁺.

(2S,3R)-2-((R)-2-phenylmorpholine-4-carbonyl)pyrrolidin-3-yl 4-((1-(2,6-dioxopiperidin-3-yl)-3-methyl-2-oxo-2,3-dihydro-1H-benzo[d]imidazol-5-yl)methyl)piperidine-1-carboxylate (C6.3). TFA (0.2 mL) was added to a solution of C6.3 (40 mg, 0.06 mmol) in DCM (1 mL) and the mixture was stirred at room temperature for 1.5 hours. The mixture was then washed with three portions of aqueous 1M NaHSO₄, three portions of saturated aqueous NaHCO₃ and two portions of brine. The organic layer was dried over MgSO₄ and concentrated in vacuo. The residue was used to next step reaction without further purification to give C6.3. ES/MS: m/z=659.3 [M+H]⁺.

(2S,3R)-1-((S)-2-((tert-butoxycarbonyl)amino)-3,3-dimethylbutanoyl)-2-((R)-2-phenylmorpholine-4-carbonyl)pyrrolidin-3-yl 4-((1-(2,6-dioxopiperidin-3-yl)-3-methyl-2-oxo-2,3-dihydro-1H-benzo[d]imidazol-5-yl)methyl)piperidine-1-carboxylate (C6.4). (2S)-2-(tert-butoxycarbonylamino)-3,3-dimethyl-butanoic acid (25 mg, 0.108 mmol), C6.3 (40 mg, 0.56 mmol) and DIPEA (0.04 mL) in acetonitrile (1.5 mL) was added HATU (31 mg, 0.08 mmol). After stirring for 1 hour, water was added to the mixture. The mixture was transferred to a separatory funnel and extracted with EtOAc. The organic extract was washed with brine, dried over MgSO₄, filtered, and concentrated under reduced pressure. Purification of the crude residue by silica gel column chromatography afforded C6.4 ES/MS: m/z=872.3 [M+H]⁺.

2S,3R)-1-((S)-2-amino-3,3-dimethylbutanoyl)-2-((R)-2-phenylmorpholine-4-carbonyl)pyrrolidin-3-yl 4-((1-(2,6-dioxopiperidin-3-yl)-3-methyl-2-oxo-2,3-dihydro-1H-benzo[d]imidazol-5-yl)methyl)piperidine-1-carboxylate (C6.5). TFA (0.2 mL) was added to a solution of C6.4 (60 mg, 0.069 mmol) in DCM (1 mL) and the mixture was stirred at room temperature for 1.5 hours. The mixture was then washed with three portions of aqueous 1M NaHSO₄, three portions of saturated aqueous NaHCO₃ and two portions of brine. The organic layer was dried over MgSO₄ and concentrated in vacuo. The residue was purified by silica gel chromatography (EtOAc/hexanes, 0 to 100%) to give C6.5. ES/MS: m/z=773.3 [M+H]⁺.

### Preparation of 3-[5-[[1-[[(2S,3S)-1-[(2S)-2-amino-3,3-dimethyl-butanoyl]-2-[(2R)-2-phenylmorpholine-4-carbonyl]pyrrolidin-3-yl]methyl]-4-piperidyl]methyl]-3-methyl-2-oxo-benzimidazol-1-yl]piperidine-2,6-dione (C7.1)

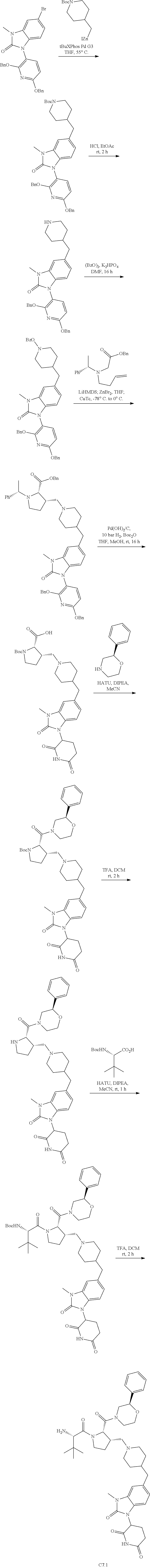

Tert-butyl 4-[[1-(2,6-dibenzyloxy-3-pyridyl)-3-methyl-2-oxo-benzimidazol-5-yl]methyl]piperidine-1-carboxylate. A stirred suspension of 5-bromo-1-(2,6-dibenzyloxy-3-pyridyl)-3-methyl-benzimidazol-2-one (1.5 g, 2.9 mmol) and tBuXPhosPdG3 (231 mg, 0.29 mmol) in THF (30 mL) was degassed with argon for 10 minutes, then was treated with (1-tert-butoxycarbonyl-4-piperidyl)methyl-iodo-zinc (0.5 M in THF, 8.7 mL, 4.36 mmol) and heated at 55 degree Celsius for 2 hours. The mixture was cooled to room temperature, and quenched with saturate aqueous bicarbonate (30 mL). The biphasic layers were separated, and the aqueous layer was extracted with ethyl acetate (3×30 mL). The combined organic layers were dried over MgSO₄, concentrated, and the resulting crude residue was purified on silica to using ethyl acetate and hexanes as eluents to give tert-butyl 4-[[1-(2,6-dibenzyloxy-3-pyridyl)-3-methyl-2-oxo-benzimidazol-5-yl]methyl]piperidine-1-carboxylate. ES/MS: m/z=635.4 [M+H]⁺.

1-(2,6-dibenzyloxy-3-pyridyl)-3-methyl-5-(4-piperidylmethyl)benzimidazol-2-one. tert-butyl 4-[[1-(2,6-dibenzyloxy-3-pyridyl)-3-methyl-2-oxo-benzimidazol-5-yl]methyl]piperidine-1-carboxylate (1.2 g, 1.93 mmol) in ethyl acetate (200 mL) was treated with 4M HCl in ethyl acetate (50 mL, 201 mmol) at room temperature and was stirred for 72 hours. The mixture was cooled to 0 degree Celsius and was basified with triethylamine. The resulting slurry was washed with water (2×100 mL), brine (1×100 mL), and the organic layer was dried over MgSO₄, concentrated to give crude residue which was purified on silica using methanol and dichloromethane as eluents to give 1-(2,6-dibenzyloxy-3-pyridyl)-3-methyl-5-(4-piperidylmethyl)benzimidazol-2-one. ES/MS m/z: [M+H⁺] 535.2

[4-[[1-(2,6-dibenzyloxy-3-pyridyl)-3-methyl-2-oxo-benzimidazol-5-yl]methyl]-1-piperidyl]benzoate. A stirred suspension of dibenzoyl peroxide (75% w/w water, 487 mg, 1.51 mmol) and potassium phosphate dibasic (364 mg, 2.09 mmol) in DMF (17.9 mL) was treated with 1-(2,6-dibenzyloxy-3-pyridyl)-3-methyl-5-(4-piperidylmethyl)benzimidazol-2-one (895 mg, 1.67 mmol) and stirred at room temperature for 72 hours. The mixture was quenched with water (10 mL) then extracted with dichloromethane (3×30 mL). The combined organic layers was washed with water (3×30 mL), brine (1×30 mL), dried over MgSO₄, and concentrated. The crude residue was purified on silica using ethyl acetate and hexanes as eluents to give [4-[[1-(2,6-dibenzyloxy-3-pyridyl)-3-methyl-2-oxo-benzimidazol-5-yl]methyl]-1-piperidyl]benzoate. ES/MS: m/z=655.3 [M+H]⁺.

Benzyl (2S,3S)-3-[[4-[[1-(2,6-dibenzyloxy-3-pyridyl)-3-methyl-2-oxo-benzimidazol-5-yl]methyl]-1-piperidyl]methyl]-1-[(1S)-1-phenylethyl]pyrrolidine-2-carboxylate. A stirred solution of benzyl 2-[but-3-enyl-[(1S)-1-phenylethyl]amino]acetate (340 mg, 1.05 mmol) in THF was degassed with argon for 10 minutes, cooled to −78 degree Celsius, then treated with LiHMDS (1.5 M in THF, 1.05 mL, 1.58 mmol). The mixture was stirred at same temperature for 10 minutes, then warmed to 0 degree Celsius and stirred for 30 minutes. The mixture was cooled to −78 degree Celsius, then treated with a solution of argon-degassed zinc bromide (709 mg, 3.15 mmol) in THF (3 mL). The mixture was stirred at same temperature for 10 minutes, warmed to room temperature over 1 hour, then was added dropwise to a argon-degassed solution of [4-[[1-(2,6-dibenzyloxy-3-pyridyl)-3-methyl-2-oxo-benzimidazol-5-yl]methyl]-1-piperidyl]benzoate (350 mg, 0.535 mmol) in THF (6 mL) at −78 degree Celsius. The resulting mixture was stirred at same temperature for 10 minutes, then warmed slowly to 0 degree Celsius over 1 hour. The mixture was quenched with saturated aqueous ammonium chloride at 0 degree Celsius, then extracted with ethyl acetate (3×20 mL). The combined organic layers was dried over MgSO₄, concentrated, and the resulting crude residue was purified by silica using dichloromethane and methanol as eluents to give benzyl (2S,3S)-3-[[4-[[1-(2,6-dibenzyloxy-3-pyridyl)-3-methyl-2-oxo-benzimidazol-5-yl]methyl]-1-piperidyl]methyl]-1-[(1S)-1-phenylethyl]pyrrolidine-2-carboxylate. ES/MS m/z: [M+H⁺] 856.3.

(2S,3S)-1-tert-butoxycarbonyl-3-[[4-[[1-(2,6-dioxo-3-piperidyl)-3-methyl-2-oxo-benzimidazol-5-yl]methyl]-1-piperidyl]methyl]pyrrolidine-2-carboxylic acid. A stirred suspension of benzyl (2S,3S)-3-[[4-[[1-(2,6-dibenzyloxy-3-pyridyl)-3-methyl-2-oxo-benzimidazol-5-yl]methyl]-1-piperidyl]methyl]-1-[(1S)-1-phenylethyl]pyrrolidine-2-carboxylate (250 mg, 0.292 mmol), palladium hydroxide on carbon (20% w/w, 820 mg, 1.17 mmol), and di-tert-butyl dicarbonate (127 mg, 0.584 mmol) in THF (5.5 mL) and MeOH (5.5 mL) was placed under 10 bar hydrogen gas, and continued to stir for 16 hours. The mixture was filtered over celite, and the filtrate was concentrated to give (2S,3S)-1-tert-butoxycarbonyl-3-[[4-[[1-(2,6-dioxo-3-piperidyl)-3-methyl-2-oxo-benzimidazol-5-yl]methyl]-1-piperidyl]methyl]pyrrolidine-2-carboxylic acid. ES/MS m/z: [M+H⁺] 584.1

Tert-butyl (2S,3S)-3-[[4-[[1-(2,6-dioxo-3-piperidyl)-3-methyl-2-oxo-benzimidazol-5-yl]methyl]-1-piperidyl]methyl]-2-[(2R)-2-phenylmorpholine-4-carbonyl]pyrrolidine-1-carboxylate. A stirred solution of (2S,3S)-1-tert-butoxycarbonyl-3-[[4-[[1-(2,6-dioxo-3-piperidyl)-3-methyl-2-oxo-benzimidazol-5-yl]methyl]-1-piperidyl]methyl]pyrrolidine-2-carboxylic acid (170 mg, 0.292 mmol), (2R)-2-phenylmorpholine (76.3 mg, 0.467 mmol), and DIPEA (0.254 mL, 1.46 mmol) in MeCN (2.5 mL) and DMF (0.5 mL) was treated with HATU (76.3 mg, 0.467 mmol) at room temperature, and was stirred for 1 hour. The mixture was concentrated then purified directly on C18 reverse phase HPLC using MeCN/H₂O/0.1% TFA as eluents to give tert-butyl (2S,3S)-3-[[4-[[1-(2,6-dioxo-3-piperidyl)-3-methyl-2-oxo-benzimidazol-5-yl]methyl]-1-piperidyl]methyl]-2-[(2R)-2-phenylmorpholine-4-carbonyl]pyrrolidine-1-carboxylate. ES/MS m/z: [M+H⁺] 729.1

3-[3-methyl-2-oxo-5-[[1-[[(2S,3S)-2-[(2R)-2-phenylmorpholine-4-carbonyl]pyrrolidin-3-yl]methyl]-4-piperidyl]methyl]benzimidazol-1-yl]piperidine-2,6-dione. A stirred solution of tert-butyl (2S,3S)-3-[[4-[[1-(2,6-dioxo-3-piperidyl)-3-methyl-2-oxo-benzimidazol-5-yl]methyl]-1-piperidyl]methyl]-2-[(2R)-2-phenylmorpholine-4-carbonyl]pyrrolidine-1-carboxylate (172 mg, 0.236 mmol) in DCM (2 mL) was treated with TFA (0.2 mL) at room temperature. After 1 h, the mixture was concentrated to afford crude 3-[3-methyl-2-oxo-5-[[1-[[(2S,3S)-2-[(2R)-2-phenylmorpholine-4-carbonyl]pyrrolidin-3-yl]methyl]-4-piperidyl]methyl]benzimidazol-1-yl]piperidine-2,6-dione, which was used directly without further purification. ES/MS m/z: [M+H⁺] 629.2

Tert-butyl N-[(1S)-1-[(2S,3S)-3-[[4-[[1-(2,6-dioxo-3-piperidyl)-3-methyl-2-oxo-benzimidazol-5-yl]methyl]-1-piperidyl]methyl]-2-[(2R)-2-phenylmorpholine-4-carbonyl]pyrrolidine-1-carbonyl]-2,2-dimethyl-propyl]carbamate. A stirred solution of 3-[3-methyl-2-oxo-5-[[1-[[(2S,3S)-2-[(2R)-2-phenylmorpholine-4-carbonyl]pyrrolidin-3-yl]methyl]-4-piperidyl]methyl]benzimidazol-1-yl]piperidine-2,6-dione (148 mg, 0.236 mmol), (2S)-2-(tertbutoxycarbonylamino)-3,3-dimethyl-butanoic acid (60 mg, 0.26 mmol), and DIPEA (0.41 mL, 2.36 mmol) in MeCN (2.4 mL) was treated with HATU (98.6 mg, 0.26 mmol) at room temperature. After 1 hour the mixture was concentrated and redissolved in EtOAc (10 mL), which was subsequently washed 1M aqueous HCl (5 mL), saturated aqueous sodium bicarbonate (5 mL), dried over MgSO₄, then concentrated. The resulting crude tert-butyl N-[(1S)-1-[(2S,3S)-3-[[4-[[1-(2,6-dioxo-3-piperidyl)-3-methyl-2-oxo-benzimidazol-5-yl]methyl]-1-piperidyl]methyl]-2-[(2R)-2-phenylmorpholine-4-carbonyl]pyrrolidine-1-carbonyl]-2,2-dimethyl-propyl]carbamate was used directly without further purification. ES/MS m/z: [M+H⁺] 842.2

3-[5-[[1-[[(2S,3S)-1-[(2S)-2-amino-3,3-dimethyl-butanoyl]-2-[(2R)-2-phenylmorpholine-4-carbonyl]pyrrolidin-3-yl]methyl]-4-piperidyl]methyl]-3-methyl-2-oxo-benzimidazol-1-yl]piperidine-2,6-dione (C7.1). A stirred solution of tert-butyl N-[(1S)-1-[(2S,3S)-3-[[4-[[1-(2,6-dioxo-3-piperidyl)-3-methyl-2-oxo-benzimidazol-5-yl]methyl]-1-piperidyl]methyl]-2-[(2R)-2-phenylmorpholine-4-carbonyl]pyrrolidine-1-carbonyl]-2,2-dimethyl-propyl]carbamate (199 mg, 0.236 mmol) in DCM (2 mL) was treated with TFA (0.2 mL) at room temperature. After 1 hour the mixture was concentrated to give crude 3-[5-[[1-[[(2S,3S)-1-[(2S)-2-amino-3,3-dimethyl-butanoyl]-2-[(2R)-2-phenylmorpholine-4-carbonyl]pyrrolidin-3-yl]methyl]-4-piperidyl]methyl]-3-methyl-2-oxo-benzimidazol-1-yl]piperidine-2,6-dione, which was used directly without further purification. ES/MS m/z: [M+H⁺] 742.4.

### Preparation of (6R,7S)-4-((2S,3S)-1-((S)-2-amino-3,3-dimethylbutanoyl)-3-((4-((1-(2,6-dioxopiperidin-3-yl)-3-methyl-2-oxo-2,3-dihydro-1H-benzo[d]imidazol-5-yl)methyl)piperidin-1-yl)methyl)pyrrolidine-2-carbonyl)-6-phenyl-4-azaspiro[2.4]heptane-7-carbonitrile or (6S,7R)-4-((2S,3S)-1-((S)-2-amino-3,3-dimethylbutanoyl)-3-((4-((1-(2,6-dioxopiperidin-3-yl)-3-methyl-2-oxo-2,3-dihydro-1H-benzo[d]imidazol-5-yl)methyl)piperidin-1-yl)methyl)pyrrolidine-2-carbonyl)-6-phenyl-4-azaspiro[2.4]heptane-7-carbonitrile (C7.2)

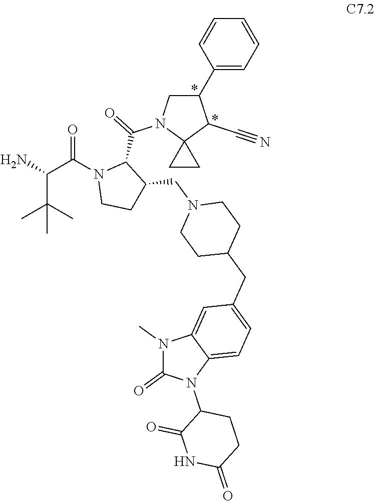 [SMILES: Cn1c(=O)n(C2CCC(=O)NC2=O)c2ccc(CC3CCN(C[C@@H]4CCN(C(=O)[C@@H](N)C(C)(C)C)[C@@H]4C(=O)N4CC(c5ccccc5)C(C#N)C45CC5)CC3)cc21]

(6R,7S)-4-((2S,3S)-1-((S)-2-amino-3,3-dimethylbutanoyl)-3-((4-((1-(2,6-dioxopiperidin-3-yl)-3-methyl-2-oxo-2,3-dihydro-1H-benzo[d]imidazol-5-yl)methyl)piperidin-1-yl)methyl)pyrrolidine-2-carbonyl)-6-phenyl-4-azaspiro[2.4]heptane-7-carbonitrile or (6S,7R)-4-((2S,3S)-1-((S)-2-amino-3,3-dimethylbutanoyl)-3-((4-((1-(2,6-dioxopiperidin-3-yl)-3-methyl-2-oxo-2,3-dihydro-1H-benzo[d]imidazol-5-yl)methyl)piperidin-1-yl)methyl)pyrrolidine-2-carbonyl)-6-phenyl-4-azaspiro[2.4]heptane-7-carbonitrile (C7.2). C7.2 was prepared analogously to C7.1, using D4.3 instead of (R)-2-phenylmorpholine. ES/MS m/z: [M+H⁺] 777.4.

### Preparation of 3-[5-[[4-[(2S,3R)-1-[(2S)-2-amino-3,3-dimethyl-butanoyl]-2-[(2R)-2-phenylmorpholine-4-carbonyl]pyrrolidin-3-yl]oxy-1-piperidyl]methyl]-3-methyl-2-oxo-benzimidazol-1-yl]piperidine-2,6-dione (C8.1)

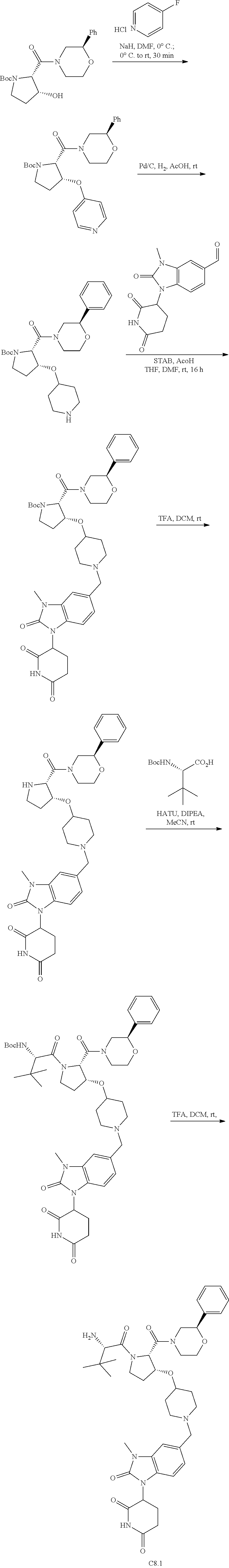

Tert-butyl (2S,3R)-2-[(2R)-2-phenylmorpholine-4-carbonyl]-3-(4-pyridyloxy)pyrrolidine-1-carboxylate. A stirred solution of tert-butyl (2S,3R)-3-hydroxy-2-[(2R)-2-phenylmorpholine-4-carbonyl]pyrrolidine-1-carboxylate (1.45 g, 3.85 mmol) in DMF (32 mL) at 0 degree Celsius was treated with sodium hydride (60% w/w, 1.18 g, 30.8 mmol). The mixture was stirred for 30 minutes at same temperature, then was treated with 4-fluoropyridine hydrochloride (1.54 g, 11.6 mmol) and warmed to room temperature over 30 minutes. The mixture was quenched with saturated aqueous ammonium chloride (10 mL), then was diluted with water (100 mL). The mixture was extracted with DCM (3×50 mL), and the combined organic layers were washed with water (3×100 mL), brine (1×100 mL), dried over MgSO₄, then concentrated. The crude residue was purified on silica using DCM and MeOH as eluents to give tert-butyl (2S,3R)-2-[(2R)-2-phenylmorpholine-4-carbonyl]-3-(4-pyridyloxy)pyrrolidine-1-carboxylate. ES/MS m/z: [M+H⁺]454.1.

Tert-butyl (2S,3R)-2-[(2R)-2-phenylmorpholine-4-carbonyl]-3-(4-piperidyloxy)pyrrolidine-1-carboxylate. A stirred suspension of tert-butyl (2S,3R)-2-[(2R)-2-phenylmorpholine-4-carbonyl]-3-(4-pyridyloxy)pyrrolidine-1-carboxylate (100 mg, 0.22 mmol) and palladium on carbon (10% w/w, 235 mg, 0.22 mmol) in AcOH (2.2 mL) was placed under an atmosphere of hydrogen gas for 16 hours. The mixture was filtered over celite, and the filtrate was concentrated then purified on silica using DCM and MeOH as eluents to give tert-butyl (2S,3R)-2-[(2R)-2-phenylmorpholine-4-carbonyl]-3-(4-piperidyloxy)pyrrolidine-1-carboxylate. ES/MS m/z: [M+H⁺] 460.1.

Tert-butyl (2S,3R)-3-[[1-[[1-(2,6-dioxo-3-piperidyl)-3-methyl-2-oxo-benzimidazol-5-yl]methyl]-4-piperidyl]oxy]-2-[(2R)-2-phenylmorpholine-4-carbonyl]pyrrolidine-1-carboxylate. A stirred solution of tert-butyl (2S,3R)-2-[(2R)-2-phenylmorpholine-4-carbonyl]-3-(4-piperidyloxy)pyrrolidine-1-carboxylate (56 mg, 0.122 mmol), 1-(2,6-dioxo-3-piperidyl)-3-methyl-2-oxo-benzimidazole-5-carbaldehyde (70 mg, 0.244 mmol), and acetic acid (0.021 mL, 0.366 mmol) in THF (0.5 mL) and DMF (0.5 mL) was treated with sodium triacetoxyborohydride (77.5 mg, 0.366 mmol) at room temperature. The mixture was stirred for 16 hours, then was quenched with saturated aqueous sodium bicarbonate (5 mL) and extracted with DCM (3×10 mL). The combined organic layers were washed with water (3×20 mL), brine (1×20 mL), dried over MgSO₄, and concentrated. The crude residue was purified by C18 reverse phase HPLC using MeCN/H₂O/0.1% TFA as eluent to give tert-butyl (2S,3R)-3-[[1-[[1-(2,6-dioxo-3-piperidyl)-3-methyl-2-oxo-benzimidazol-5-yl]methyl]-4-piperidyl]oxy]-2-[(2R)-2-phenylmorpholine-4-carbonyl]pyrrolidine-1-carboxylate. ES/MS m/z: [M+H⁺] 731.2.

3-[3-methyl-2-oxo-5-[[4-[(2S,3R)-2-[(2R)-2-phenylmorpholine-4-carbonyl]pyrrolidin-3-yl]oxy-1-piperidyl]methyl]benzimidazol-1-yl]piperidine-2,6-dione. A stirred solution of tert-butyl (2S,3R)-3-[[1-[[1-(2,6-dioxo-3-piperidyl)-3-methyl-2-oxo-benzimidazol-5-yl]methyl]-4-piperidyl]oxy]-2-[(2R)-2-phenylmorpholine-4-carbonyl]pyrrolidine-1-carboxylate (30 mg, 0.041 mmol) in DCM (2 mL) was treated with TFA (0.5 mL) at room temperature. After 1 hour the mixture was concentrated to give 3-[3-methyl-2-oxo-5-[[4-[(2S,3R)-2-[(2R)-2-phenylmorpholine-4-carbonyl]pyrrolidin-3-yl]oxy-1-piperidyl]methyl]benzimidazol-1-yl]piperidine-2,6-dione, which was used directly without further purification. ES/MS m/z: [M+H⁺] 631.2.

Tert-butyl N-[(1S)-1-[(2S,3R)-3-[[1-[[1-(2,6-dioxo-3-piperidyl)-3-methyl-2-oxo-benzimidazol-5-yl]methyl]-4-piperidyl]oxy]-2-[(2R)-2-phenylmorpholine-4-carbonyl]pyrrolidine-1-carbonyl]-2,2-dimethyl-propyl]carbamate. A stirred solution of tert-butyl (2S,3R)-3-[[1-[[1-(2,6-dioxo-3-piperidyl)-3-methyl-2-oxo-benzimidazol-5-yl]methyl]-4-piperidyl]oxy]-2-[(2R)-2-phenylmorpholine-4-carbonyl]pyrrolidine-1-carboxylate (30 mg, 0.041 mmol), (2S)-2-(tert-butoxycarbonylamino)-3,3-dimethyl-butanoic acid (10.4 mg, 0.0451 mmol), and DIPEA (0.071 mL, 0.41 mmol) in MeCN (1 mL) was treated with HATU (15.6 mg, 0.041 mmol). After 1 hour the mixture was concentrated and redissolved in DCM (10 mL). The organic layer was washed with 1M aqueous HCl (5 mL), saturated aqueous sodium bicarbonate (5 mL), dried over MgSO₄, and concentrated. The crude tert-butyl N-[(1S)-1-[(2S,3R)-3-[[1-[[1-(2,6-dioxo-3-piperidyl)-3-methyl-2-oxo-benzimidazol-5-yl]methyl]-4-piperidyl]oxy]-2-[(2R)-2-phenylmorpholine-4-carbonyl]pyrrolidine-1-carbonyl]-2,2-dimethyl-propyl]carbamate was used directly without further purification. ES/MS m/z: [M+H⁺] 844.2.

3-[5-[[4-[(2S,3R)-1-[(2S)-2-amino-3,3-dimethyl-butanoyl]-2-[(2R)-2-phenylmorpholine-4-carbonyl]pyrrolidin-3-yl]oxy-1-piperidyl]methyl]-3-methyl-2-oxo-benzimidazol-1-yl]piperidine-2,6-dione (C8.1). A stirred solution of tert-butyl N-[(1S)-1-[(2S,3R)-3-[[1-[[1-(2,6-dioxo-3-piperidyl)-3-methyl-2-oxo-benzimidazol-5-yl]methyl]-4-piperidyl]oxy]-2-[(2R)-2-phenylmorpholine-4-carbonyl]pyrrolidine-1-carbonyl]-2,2-dimethyl-propyl]carbamate (34.6 mg, 0.041 mmol) in DCM (1 mL) was treated with TFA (0.2 mL) at room temperature. After 1 hour the mixture was concentrated to give crude 3-[5-[[4-[(2S,3R)-1-[(2S)-2-amino-3,3-dimethyl-butanoyl]-2-[(2R)-2-phenylmorpholine-4-carbonyl]pyrrolidin-3-yl]oxy-1-piperidyl]methyl]-3-methyl-2-oxo-benzimidazol-1-yl]piperidine-2,6-dione, which was used directly without further purification. ES/MS m/z: [M+H⁺] 744.2.

### Preparation of 3-(5-((4-(2-(((2S,3R)-1-((S)-2-amino-3,3-dimethylbutanoyl)-2-((R)-2-phenylmorpholine-4-carbonyl)pyrrolidin-3-yl)oxy)acetyl)piperazin-1-yl)methyl)-3-methyl-2-oxo-2,3-dihydro-1H-benzo[d]imidazol-1-yl)piperidine-2,6-dione (C9.1)

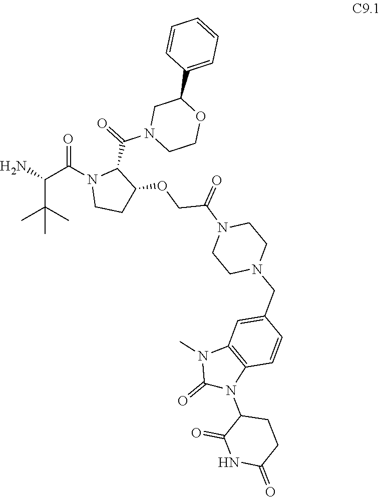 [SMILES: Cn1c(=O)n(C2CCC(=O)NC2=O)c2ccc(CN3CCN(C(=O)CO[C@@H]4CCN(C(=O)[C@@H](N)C(C)(C)C)[C@@H]4C(=O)N4CCO[C@H](c5ccccc5)C4)CC3)cc21]

3-(5-((4-(2-(((2S,3R)-1-((S)-2-amino-3,3-dimethylbutanoyl)-2-((R)-2-phenylmorpholine-4-carbonyl)pyrrolidin-3-yl)oxy)acetyl)piperazin-1-yl)methyl)-3-methyl-2-oxo-2,3-dihydro-1H-benzo[d]imidazol-1-yl)piperidine-2,6-dione (C9.1). C9.1 was prepared analogously to C2.2, using A4.10 instead of A3.4. ES/MS m/z: [M+H⁺] 787.4.

### Preparation of 2-(((2S,3R)-1-((2S)-2-(5-((diethoxyphosphoryl)fluoromethyl)benzo[b]thiophene-2-carboxamido)-3,3-dimethylbutanoyl)-2-((R)-2-phenylmorpholine-4-carbonyl)pyrrolidin-3-yl)oxy)acetic acid (C10.1)

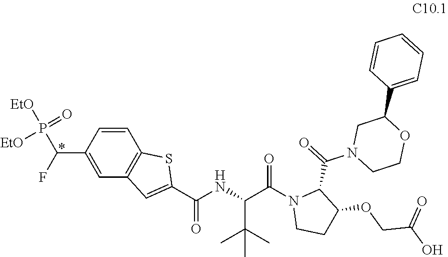 [SMILES: CCOP(=O)(OCC)C(F)c1ccc2sc(C(=O)N[C@H](C(=O)N3CC[C@@H](OCC(=O)O)[C@H]3C(=O)N3CCO[C@H](c4ccccc4)C3)C(C)(C)C)cc2c1]

2-(((2S,3R)-1-((2S)-2-(5-((diethoxyphosphoryl)fluoromethyl)benzo[b]thiophene-2-carboxamido)-3,3-dimethylbutanoyl)-2-((R)-2-phenylmorpholine-4-carbonyl)pyrrolidin-3-yl)oxy)acetic acid (C10.1). C10.1 was prepared analogously to C1.5, using B3.3 instead of B1.1. ES/MS m/z: [M+H⁺] 776.2.

### Preparation of 3-(5-((3-(2-(((2S,3R)-1-((S)-2-amino-3,3-dimethylbutanoyl)-2-((R)-2-phenylmorpholine-4-carbonyl)pyrrolidin-3-yl)oxy)acetyl)-3,8-diazabicyclo[3.2.1]octan-8-yl)methyl)-3-methyl-2-oxo-2,3-dihydro-1H-benzo[d]imidazol-1-yl)piperidine-2,6-dione (C11.1)

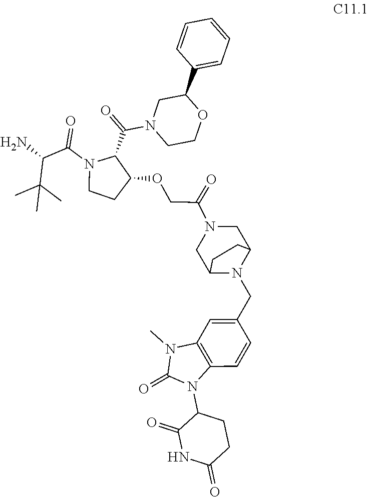 [SMILES: Cn1c(=O)n(C2CCC(=O)NC2=O)c2ccc(CN3C4CCC3CN(C(=O)CO[C@@H]3CCN(C(=O)[C@@H](N)C(C)(C)C)[C@@H]3C(=O)N3CCO[C@H](c5ccccc5)C3)C4)cc21]

3-(5-((3-(2-(((2S,3R)-1-((S)-2-amino-3,3-dimethylbutanoyl)-2-((R)-2-phenylmorpholine-4-carbonyl)pyrrolidin-3-yl)oxy)acetyl)-3,8-diazabicyclo[3.2.1]octan-8-yl)methyl)-3-methyl-2-oxo-2,3-dihydro-1H-benzo[d]imidazol-1-yl)piperidine-2,6-dione (C11.1). C11.1 was prepared analogously to C2.2, using A1.2 instead of A3.4. ES/MS m/z: [M+H⁺] 813.4.

### Preparation of 3-(5-((3-((2S,3S)-1-((S)-2-amino-3,3-dimethylbutanoyl)-2-((R)-2-phenylmorpholine-4-carbonyl)pyrrolidin-3-yl)azetidin-1-yl)methyl)-3-methyl-2-oxo-2,3-dihydro-1H-benzo[d]imidazol-1-yl)piperidine-2,6-dione (C12.1)

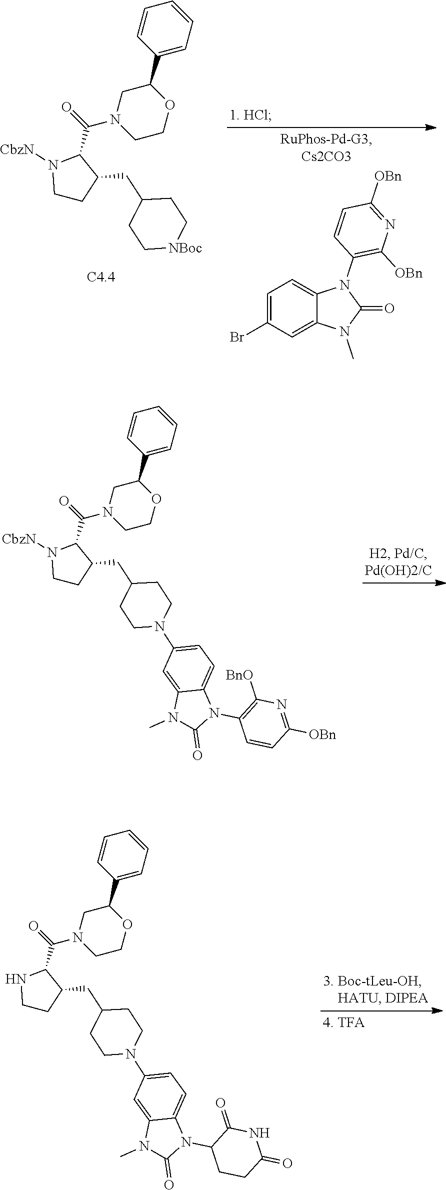

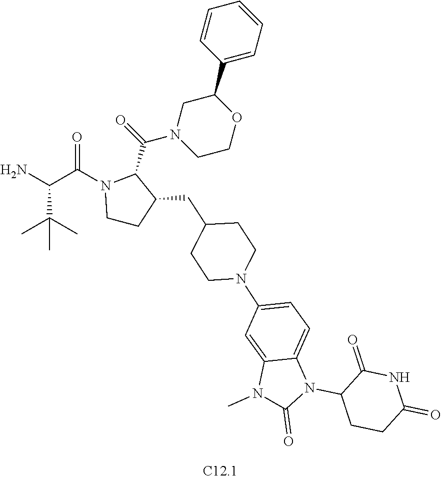 [SMILES: Cn1c(=O)n(C2CCC(=O)NC2=O)c2ccc(N3CCC(C[C@@H]4CCN(C(=O)[C@@H](N)C(C)(C)C)[C@@H]4C(=O)N4CCO[C@H](c5ccccc5)C4)CC3)cc21]

Step 1. C4.4 (300 mg, 0.61 mmol) was suspended in 4.0N HCl (4 mL). After stirring for 2 hours, the mixture was concentrated, then resuspended in dioxane (4.0 mL). RuPhos-Pd-G3 (45 mg, 0.06 mmol), 5-bromo-1-(2,6-dibenzyloxy-3-pyridyl)-3-methyl-benzimidazol-2-one (347 mg, 0.67 mmol), and cesium carbonate (596 mg, 1.8 mmol) were added. The mixture was heated to 110° C. overnight, then cooled to room temperature. Water and EtOAc were added, and the organic layer was separated and concentrated. The residue was purified by flash column chromatography to afford benzyl (2S,3R)-3-[[1-[1-(2,6-dibenzyloxy-3-pyridyl)-3-methyl-2-oxo-benzimidazol-5-yl]-4-piperidyl]methyl]-2-[(2R)-2-phenylmorpholine-4-carbonyl]pyrrolidine-1-carboxylate.

Step 2. A mixture of benzyl (2S,3R)-3-[[1-[1-(2,6-dibenzyloxy-3-pyridyl)-3-methyl-2-oxo-benzimidazol-5-yl]-4-piperidyl]methyl]-2-[(2R)-2-phenylmorpholine-4-carbonyl]pyrrolidine-1-carboxylate (250 mg, 0.27 mmol) Pd/C (10% w/w, 143 mg, 0.135 mmol), and Pd(OH)₂/C (20% w/w, 947 mg, 1.35 mmol) were stirred under an atmosphere of hydrogen for 2 days. The mixture was filtered over Celite and concentrated to afford 3-[3-methyl-2-oxo-5-[4-[[(2S,3R)-2-[(2R)-2-phenylmorpholine-4-carbonyl]pyrrolidin-3-yl]methyl]-1-piperidyl]benzimidazol-1-yl]piperidine-2,6-dione. ES/MS: m/z=615.2 [M+H]⁺.

Step 3. 3-[3-methyl-2-oxo-5-[4-[[(2S,3R)-2-[(2R)-2-phenylmorpholine-4-carbonyl]pyrrolidin-3-yl]methyl]-1-piperidyl]benzimidazol-1-yl]piperidine-2,6-dione (50 mg, 0.08 mmol), (2S)-2-(tert-butoxycarbonylamino)-3,3-dimethyl-butanoic acid (22 mg, 0.098 mmol), HATU (43 mg, 0.11 mmol), and DIPEA (0.07 mL, 0.41 mmol) were stirred in MeCN (1 mL) for 2 hours. The mixture was concentrated and purified by flash column chromatography to afford tert-butyl N-[(1S)-1-[(2S,3R)-3-[[1-[1-(2,6-dioxo-3-piperidyl)-3-methyl-2-oxo-benzimidazol-5-yl]-4-piperidyl]methyl]-2-[(2R)-2-phenylmorpholine-4-carbonyl]pyrrolidine-1-carbonyl]-2,2-dimethyl-propyl]carbamate. ES/MS: m/z=828.4 [M+H]⁺.

Step 4. tert-butyl N-[(1S)-1-[(2S,3R)-3-[[1-[1-(2,6-dioxo-3-piperidyl)-3-methyl-2-oxo-benzimidazol-5-yl]-4-piperidyl]methyl]-2-[(2R)-2-phenylmorpholine-4-carbonyl]pyrrolidine-1-carbonyl]-2,2-dimethyl-propyl]carbamate (53 mg, 0.06 mmol) was suspended in DCM/TFA (5:1 (2 mL). After stirring for 1 hour, the mixture was concentrated to afford 3-(5-((3-((2S,3S)-1-((S)-2-amino-3,3-dimethylbutanoyl)-2-((R)-2-phenylmorpholine-4-carbonyl)pyrrolidin-3-yl)azetidin-1-yl)methyl)-3-methyl-2-oxo-2,3-dihydro-1H-benzo[d]imidazol-1-yl)piperidine-2,6-dione (C12.1). ES/MS: m/z=728.4 [M+H]⁺.

### Preparation of tert-butyl 6-(pyridin-3-yl)-4-azaspiro[2.4]heptane-4-carboxylate (SFC peak 1, D1.1) and tert-butyl 6-(pyridin-3-yl)-4-azaspiro[2.4]heptane-4-carboxylate (SFC peak 2, D1.2)

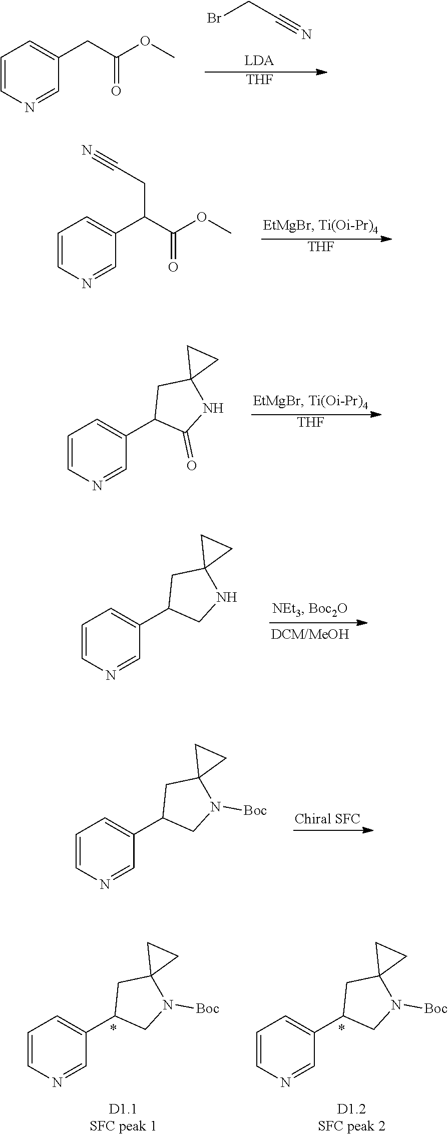

methyl 3-cyano-2-(pyridin-3-yl)propanoate. To a solution of methyl 2-(pyridin-3-yl)acetate (20.0 g, 132.30 mmol, 1.00 eq) in THF (400.0 mL) was added LDA (99.3 mL, 198.45 mmol, 1.50 eq, 2.0 M in THF) dropwise at −65° C. under N2 atmosphere. The reaction was stirred at −65° C. for 1 h. 2-bromoacetonitrile (19.0 g, 158.76 mmol, 1.20 eq) was added to the mixture drop-wise at −65° C. under N2 atmosphere, then the mixture was stirred at −65° C. for another 1 h. To the mixture was added ice-cold NH₄Cl (aq, 500.0 mL), then was extracted with EtOAc (500.0 mL×2). The organic layer was washed with brine, dried and concentrated. The residue was purified by silica gel chromatography with (PE:EA=3:1 to 1:1) to afford methyl 3-cyano-2-(pyridin-3-yl)propanoate. MS (ESI) m/z: 191.0 [M+H]+. 1H NMR (400 MHz, DMSO-d6) 8.58-8.54 (m, 2H), 7.79-7.76 (m, 1H), 7.44-7.41 (m, 1H), 4.30 (t, J=7.2 Hz, 1H), 3.65 (s, 3H), 3.24-3.11 (m, 2H).

6-(pyridin-3-yl)-4-azaspiro[2.4]heptan-5-one. To a solution of methyl 3-cyano-2-(pyridin-3-yl)propanoate (38.5 g, 202.42 mmol, 1.00 eq) and Ti(OiPr)₄ (69.0 g, 242.90 mmol, 1.20 eq) in THF (500.0 mL) was added EtMgBr (455.5 mL, 455.44 mmol, 2.25 eq, 1.0 M in THF) at 0° C. dropwise. After addition, the reaction was stirred at 0° C. for 1 h and room temperature for another 15 h. To the mixture was added ice-water (300.0 mL), filtered and the filtrate was extracted with EA (600.0 mL×3), the organic layer was dried and concentrated. The residue was then purified by column chromatography with (DCM:MeOH=50:1 to 20:1) to afford 6-(pyridin-3-yl)-4-azaspiro[2.4]heptan-5-one. MS (ESI) m/z: 189.0 [M+H]+. 1H NMR (400 MHz, DMSO-d6) 8.51 (d, J=2.0 Hz, 1H), 8.46 (dd, J=4.8 Hz, 1.6 Hz, 1H), 7.99 (s, 1H), 7.72-7.69 (m, 1H), 7.38-7.35 (m, 1H), 3.86 (t, J=8.8 Hz, 1H), 2.49-2.42 (m, 1H), 2.28-2.22 (m, 1H), 0.85-0.66 (m, 4H).

6-(pyridin-3-yl)-4-azaspiro[2.4]heptane. To a solution of 6-(pyridin-3-yl)-4-azaspiro[2.4]heptan-5-one (19.2 g, 102.00 mmol, 1.00 eq) in THF (200.0 mL) was added borane-methyl sulfide complex (51.0 mL, 510.00 mmol, 5.00 eq, 10.0 M in THF) at 0° C. under argon. Then the mixture was stirred at 80° C. for 12 h. The mixture was then cooled to 0° C., MeOH (100.0 mL) was added to the mixture at dropwise, then 3N HCl (50.0 mL) was added to the mixture. This mixture was stirred at reflux for 2 h then was concentrated to remove THF. Water (200.0 mL) was then added to the mixture and extracted with EA (200.0 mL×3. The aqueous phase was adjusted pH to 10-11, extracted with EA (600.0 mL×3), and the organic layers were dried over Na₂SO₄ and concentrated to afford 6-(pyridin-3-yl)-4-azaspiro[2.4]heptane which was used in the next step without further purification. MS (ESI) m/z: 175.3 [M+H]⁺.

tert-butyl 6-(pyridin-3-yl)-4-azaspiro[2.4]heptane-4-carboxylate. To a solution of 6-(pyridin-3-yl)-4-azaspiro[2.4]heptane (11.0 g, 63.13 mmol, 1.00 eq) and TEA (19.2 g, 189.38 mmol, 3.00 eq) in MeOH (100.0 mL) and DCM (100.0 mL) was added Boc₂O (16.5 g, 75.75 mmol, 1.20 eq) at 0° C. dropwise. Then the mixture was stirred at room temperature for 12 h. The mixture was concentrated, water (200.0 mL) was added, extracted with EA (200.0 mL×2), the organic layers were washed with brine, concentrated and purified by column chromatography (PE:EA=5:1 to 1:1) to afford tert-butyl 6-(pyridin-3-yl)-4-azaspiro[2.4]heptane-4-carboxylate.

SFC peak 1 tert-butyl 6-(pyridin-3-yl)-4-azaspiro[2.4]heptane-4-carboxylate (D1.1) and SFC peak 2 tert-butyl 6-(pyridin-3-yl)-4-azaspiro[2.4]heptane-4-carboxylate (D1.2). 14.2 g of racemic tert-butyl 6-(pyridin-3-yl)-4-azaspiro[2.4]heptane-4-carboxylate was separated by chiral SFC (IG, 250 mm*50 mm*10 um Daicel, 75% CO2, 25% MeOH (+0.1% 7.0 mol/1 Ammonia in MeOH), Flow: 150 mL/min, Temp: RT, Back Pressure: 100 bar) to afford D1.1 (First eluting SFC peak) and D1.2 (Second eluting SFC peak).

First eluting SFC peak (D1.1): MS (ESI) m/z: 275.1 [M+H]+. 1H NMR (400 MHz, DMSO-d6) 8.55 (d, J=2.8 Hz, 1H), 8.44 (dd, J=4.8 Hz, 1.6 Hz, 1H), 7.79-7.76 (m, 1H), 7.34 (dd, J=8.0 Hz, 4.8 Hz, 1H), 3.89 (dd, J=10.0 Hz, 7.2 Hz, 1H), 3.51-3.44 (m, 1H), 3.40-3.35 (m, 1H), 2.33 (t, J=11.2 Hz, 1H), 2.05-2.00 (m, 1H), 1.80-1.70 (m, 1H), 1.37 (s, 9H), 1.33-1.31 (m, 1H), 0.53-0.50 (m, 2H).

Second eluting SFC peak (D1.2): MS (ESI) m/z: 275.1 [M+H]+. 1H NMR (400 MHz, DMSO-d6) 8.55 (d, J=2.4 Hz, 1H), 8.44 (dd, J=4.8 Hz, 1.6 Hz, 1H), 7.79-7.76 (m, 1H), 7.34 (dd, J=7.6 Hz, 4.8 Hz, 1H), 3.89 (dd, J=10.0 Hz, 7.2 Hz, 1H), 3.51-3.44 (m, 1H), 3.40-3.35 (m, 1H), 2.33 (t, J=11.2 Hz, 1H), 2.05-2.00 (m, 1H), 1.80-1.70 (m, 1H), 1.37 (s, 9H), 1.33-1.31 (m, 1H), 0.53-0.50 (m, 2H).

### Preparation of 6-(pyridin-3-yl)-4-azaspiro[2.4]heptane (D1.3)

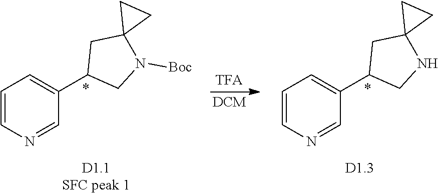 [SMILES: CC(C)(C)OC(=O)N1CC(c2cccnc2)CC12CC2 ; c1cncc(C2CNC3(CC3)C2)c1]

tert-butyl 6-(pyridin-3-yl)-4-azaspiro[2.4]heptane-4-carboxylate D1.1 (50 mg, 0.18 mmol) was suspended in DCM/TFA (5:1, 2 mL). After stirring for 30 minutes, the mixture was concentrated, resuspended in EtOAc, and washed with sat. aq. NaHCO₃. The organic layer was concentrated to afford 6-(pyridin-3-yl)-4-azaspiro[2.4]heptane D1.3 which was used without further purification. ES/MS: m/z=175.1 [M+H]+.

### Preparation of 6-(pyridin-3-yl)-4-azaspiro[2.4]heptane (D1.4)

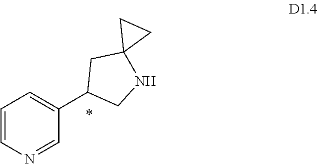 [SMILES: c1cncc(C2CNC3(CC3)C2)c1]

D1.4 was prepared analogously to D1.3 using tert-butyl D1.2 instead of D1.1. ES/MS: m/z=175.1 [M+H]+.

### Preparation of Phenylmorpholine with 4,4-difluoro-3-phenyl-piperidine (D2.1)

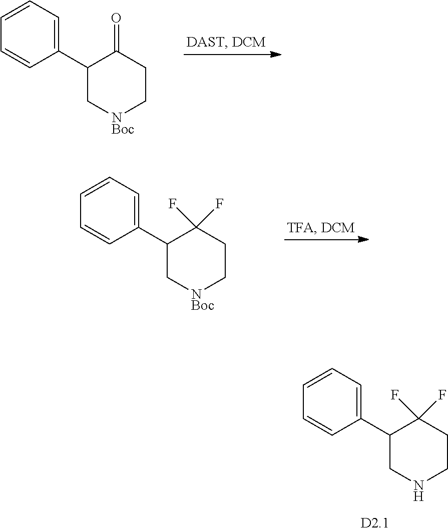 [SMILES: CC(C)(C)OC(=O)N1CCC(=O)C(c2ccccc2)C1 ; CC(C)(C)OC(=O)N1CCC(F)(F)C(c2ccccc2)C1 ; FC1(F)CCNCC1c1ccccc1]

Tert-butyl 4,4-difluoro-3-phenyl-piperidine-1-carboxylate. A stirred solution of tert-butyl 4-oxo-3-phenyl-piperidine-1-carboxylate (100 mg, 0.363 mmol) in DCM (1.8 mL) at 0 degree Celsius was treated with diethylaminosulfur trifluoride (0.096 mL, 0.726 mmol). After 6 hours at the same temperature, the mixture was quenched with addition of saturated aqueous sodium bicarbonate and layers were separated. The aqueous layer was extracted with DCM (3×5 mL), and the combined organic layers were dried over MgSO₄, concentrated. The resulting crude residue was purified on silica using ethyl acetate and hexanes as eluents to give tert-butyl 4,4-difluoro-3-phenyl-piperidine-1-carboxylate. ES/MS m/z: (M+H⁺) 298.2

Phenylmorpholine with 4,4-difluoro-3-phenyl-piperidine (D2.1). A stirred solution of tert-butyl 4,4-difluoro-3-phenyl-piperidine-1-carboxylate (69 mg, 0.232 mmol) in DCM (2.2 mL) was treated with TFA (0.2 mL) at room temperature. After 1 hour the mixture was concentrated to give 4,4-difluoro-3-phenyl-piperidine, which was used directly without further purification. ES/MS m/z: (M+H⁺) 198.2

### Preparation of 3-(5-((4-(((2S,3R)-1-((S)-2-amino-3,3-dimethylbutanoyl)-2-(4,4-difluoro-3-phenylpiperidine-1-carbonyl)pyrrolidin-3-yl)methyl)piperidin-1-yl)methyl)-3-methyl-2-oxo-2,3-dihydro-1H-benzo[d]imidazol-1-yl)piperidine-2,6-dione (D2.2)

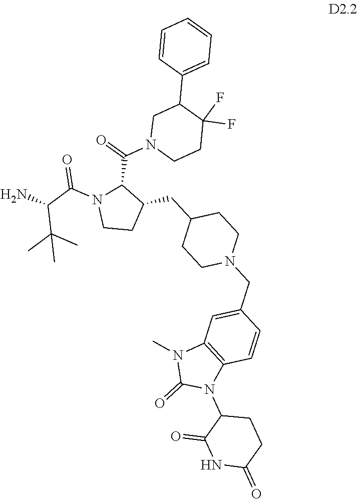 [SMILES: Cn1c(=O)n(C2CCC(=O)NC2=O)c2ccc(CN3CCC(C[C@@H]4CCN(C(=O)[C@@H](N)C(C)(C)C)[C@@H]4C(=O)N4CCC(F)(F)C(c5ccccc5)C4)CC3)cc21]

3-(5-((4-(((2S,3R)-1-((S)-2-amino-3,3-dimethylbutanoyl)-2-(4,4-difluoro-3-phenylpiperidine-1-carbonyl)pyrrolidin-3-yl)methyl)piperidin-1-yl)methyl)-3-methyl-2-oxo-2,3-dihydro-1H-benzo[d]imidazol-1-yl)piperidine-2,6-dione (D2.2). D2.2 was prepared analogously to C5.3 using D2.1 instead of (R)-2-phenylmorpholine. ES/MS m/z: (M+H⁺) 776.4.

### Preparation of (2R)-1-methyl-2-phenyl-piperazine (D3.1)

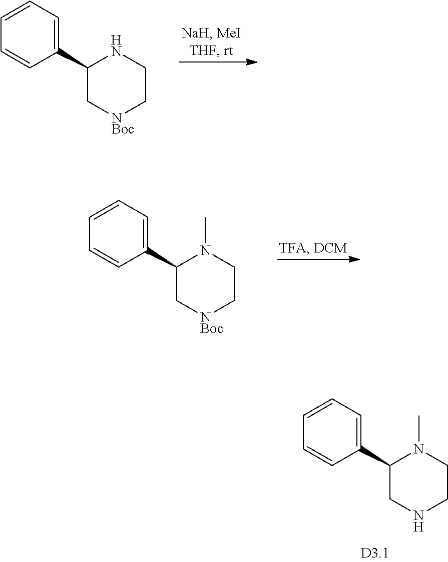 [SMILES: CC(C)(C)OC(=O)N1CCN[C@H](c2ccccc2)C1 ; CN1CCN(C(=O)OC(C)(C)C)C[C@H]1c1ccccc1 ; CN1CCNC[C@H]1c1ccccc1]

Tert-butyl (3R)-4-methyl-3-phenyl-piperazine-1-carboxylate. A stirred solution of tert-butyl (3R)-3-phenylpiperazine-1-carboxylate hydrochloride (277 mg, 0.927 mmol) in THF (8.31 mL) at 0 degree Celsius was treated with sodium hydride (60% w/w, 284 mg, 7.42 mmol). After 30 minutes at same temperature, the mixture was treated with iodomethane (0.069 mL, 1.11 mmol) then was warmed to room temperature over 16 hours. The mixture was quenched with addition of saturated aqueous ammonium chloride (10 mL), diluted with ethyl acetate (10 mL), and the layers were separated. The aqueous layer was extracted with ethyl acetate (2×10 mL), and the combined organic layers were dried over MgSO₄ then concentrated. The resulting crude residue was purified by silica using DCM and MeOH as eluents to give tert-butyl (3R)-4-methyl-3-phenyl-piperazine-1-carboxylate. ES/MS m/z: (M+1) 277.2.

(2R)-1-methyl-2-phenyl-piperazine (D3.1). A stirred solution of tert-butyl (3R)-4-methyl-3-phenyl-piperazine-1-carboxylate (170 mg, 0.615 mmol) in DCM (3 mL) was treated with TFA (0.6 mL) at room temperature. After 1 hour the mixture was concentrated to give (2R)-1-methyl-2-phenyl-piperazine, which was used directly without further purification. ES/MS m/z: (M+1) 177.2

### Preparation of 3-(5-((4-(((2S,3R)-1-((S)-2-amino-3,3-dimethylbutanoyl)-2-((R)-4-methyl-3-phenylpiperazine-1-carbonyl)pyrrolidin-3-yl)methyl)piperidin-1-yl)methyl)-3-methyl-2-oxo-2,3-dihydro-1H-benzo[d]imidazol-1-yl)piperidine-2,6-dione (D3.2)

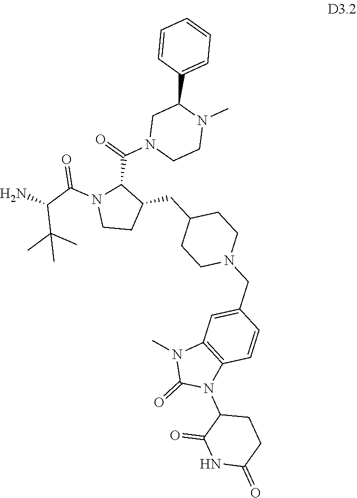 [SMILES: CN1CCN(C(=O)[C@@H]2[C@H](CC3CCN(Cc4ccc5c(c4)n(C)c(=O)n5C4CCC(=O)NC4=O)CC3)CCN2C(=O)[C@@H](N)C(C)(C)C)C[C@H]1c1ccccc1]

3-(5-((4-(((2S,3R)-1-((S)-2-amino-3,3-dimethylbutanoyl)-2-((R)-4-methyl-3-phenylpiperazine-1-carbonyl)pyrrolidin-3-yl)methyl)piperidin-1-yl)methyl)-3-methyl-2-oxo-2,3-dihydro-1H-benzo[d]imidazol-1-yl)piperidine-2,6-dione (D3.2). D3.2 was prepared analogously to C5.3 using D3.1 instead of (R)-2-phenylmorpholine. ES/MS m/z: (M+H⁺) 755.4.

### Preparation of (6R,7S)-6-phenyl-4-azaspiro[2.4]heptane-7-carbonitrile (D4.3)

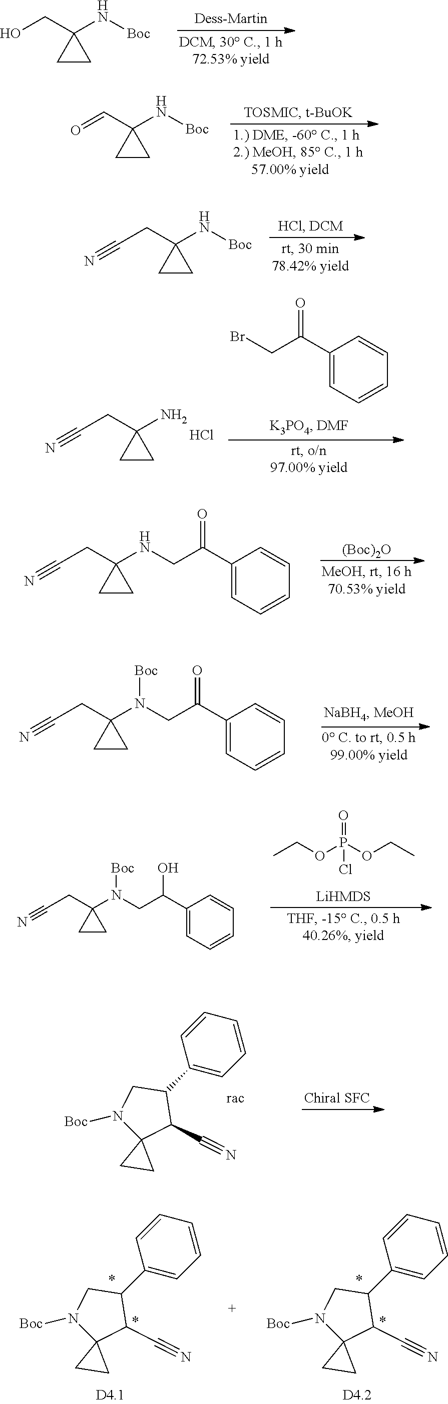

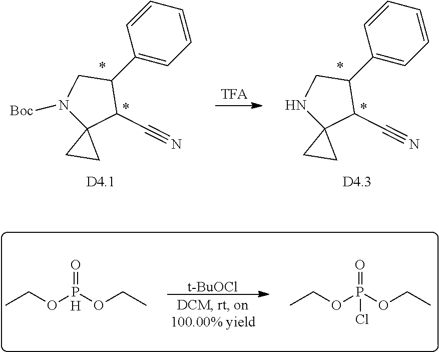

Tert-butyl (1-formylcyclopropyl)carbamate. To a stirred solution of tert-butyl (1-(hydroxymethyl)cyclopropyl)carbamate (200 g, 1068 mmol) in DCM (4 L) was slowly added Dess-Martin periodinane (453 g, 1068 mmol) at 0° C. over 10 minutes. The reaction was stirred at 30° C. for one hour. After cooling, the mixture was concentrated and the resulting was filtered and the filtrate was concentrated in vacuo to afford crude product. The crude product was purified by silica-gel chromatography to afford the title compound. ¹H NMR (400 MHz, DMSO-d₆) δ 8.99 (s, 1H), 7.53 (s, 1H), 1.38 (s, 9H), 1.36-1.35 (m, 2H), 1.15-1.12 (m, 2H).

Tert-butyl (1-(cyanomethyl)cyclopropyl)carbamate. Potassium tert-butoxide (150 g, 1318 mmol, 2.44 equiv) was suspended in anhydrous DME (600 mL) and cooled to −60° C. Tos-MIC (160 g, 805 mmol, 1.49 equiv.) was dissolved in anhydrous DME (500 mL), and this was added drop-wise to the potassium tert-butoxide solution over 2 minutes. After stirring for 20 minutes between −60° C., t tert-butyl (1-formylcyclopropyl)carbamate (100 g, 540 mmol) in anhydrous DME (500 mL), was added over 2 minutes. The reaction was stirred one hour at −60° C. to yield a thick suspension. Methanol (600 mL) was then added resulting in a clear brown solution. The cooling bath was removed, and after stirring 5 minutes in air, the reaction flask was immersed in an oil bath preheated to 85° C. The reaction was stirred for 1 hour. After cooling, the mixture was concentrated and the resulting tan solid was dissolved in water. This was extracted with ethyl acetate (500 mL×3), and these extracts were combined, washed with saturated aqueous sodium chloride (500 mL), dried over sodium sulfate, filtered and concentrated, then dissolved in dichloromethane and purified by silica-gel chromatography to afford the title compound. ¹H NMR (400 MHz, DMSO-d₆) δ 5.10 (s, 1H), 2.72 (s, 2H), 1.44 (s, 9H), 0.85-0.95 (m, 4H).

2-(1-aminocyclopropyl)acetonitrile hydrochloride. To a stirred solution of tert-butyl N-[1-(cyanomethyl) cyclopropyl]carbamate (60 g, 305.5 mmol) in DCM (620 mL) was added 4N HCl in dioxane (155 mL). The resulting mixture was stirred at room temperature for 30 min. The precipitate was collected by filtration and dried under reduced pressure to afford the title compound. LCMS (ESI) m/z=97.1 [M+H]+.

2-(1-((2-oxo-2-phenylethyl)amino)cyclopropyl)acetonitrile. To a solution of 2-(1-aminocyclopropyl)acetonitrile hydrochloride (31.7 g, 239.5 mmol) in DMF (250 mL) was added 2-bromo-1-phenylethan-1-one (47.8 g, 239.5 mmol) followed by potassium phosphate tribasic (127 g, 598.75 mmol). The mixture was stirred at room temperature for 4 h. The reaction was quenched with the addition of 1 N HCl (500 mL, pH=1) at room temperature. The aqueous solution was washed with EtOAc (2×300 mL) then basified with 1 N NaOH aqueous solution (until pH=9, 20 mL) then extracted with EtOAc (2×400 mL). The combined organic extracts were washed with brine (2×500 mL), dried over anhydrous Na₂SO₄, filtered and concentrated under reduced pressure. This residue was purified by silica gel column chromatography to give the title compound. LCMS (ESI) m/z=215.2 [M+H]+.

Tert-butyl (1-(cyanomethyl)cyclopropyl)(2-oxo-2-phenylethyl)carbamate. To a solution of 2-(1-((2-oxo-2-phenylethyl)amino)cyclopropyl)acetonitrile (50 g, 233 mmol) in MeOH (500 mL) were added di-tert-butyl dicarbonate (76 g, 350 mmol). The reaction was stirred for 16 h at room temperature. The reaction was concentrated under reduced pressure. This residue was purified by silica gel column chromatography to afford the title compound. LCMS (ES) m/z=259.2 (M-t-Bu+H).

Tert-butyl (1-(cyanomethyl)cyclopropyl)(2-hydroxy-2-phenylethyl)carbamate. To a solution of tert-butyl (1-(cyanomethyl)cyclopropyl)(2-oxo-2-phenylethyl) carbamate (39 g, 124.5 mmol) in MeOH (700 mL) was added sodium borohydride (4.7 g, 124.05 mmol) at 0° C. The reaction was stirred for 0.5 h at room temperature. The reaction diluted with water and extracted with EtOAc. The organic layer was washed with brine, dried over anhydrous Na₂SO₄, filtered and concentrated under reduced pressure to afford the title compound. LCMS (ES) m/z=339.3 (M+Na).

Diethyl phosphorochloridate. To a solution of diethyl phosphonate (10 g, 72.4 mmol) in dry DCM (150 mL) were added t-BuOCl (23.6 g, 217.2 mmol) at room temperature under Ar. The reaction was stirred for 16 h at room temperature. The reaction was concentrated under reduced pressure to afford the title compound which was used without further purification.

Rac-tert-butyl (6S,7R)-7-cyano-6-phenyl-4-azaspiro[2.4]heptane-4-carboxylate. To a solution of tert-butyl (1-(cyanomethyl)cyclopropyl)(2-hydroxy-2-phenylethyl)carbamate (21.6 g, 68.3 mmol) in THF (1000 mL) was added diethyl phosphorochloridate (12.4 g, 71.7 mmol) and 1M LiHMDS (170 mL, 171 mmol) at −15° C. under Ar. The reaction was stirred for 0.5 h at −15° C. The reaction diluted with water and extracted with EtOAc. The organic layer was washed with brine, dried over anhydrous Na₂SO₄, filtered and concentrated under reduced pressure. This residue was purified by TLC (PE/EA=10/1) to give the title compound. LCMS (ES) m/z=243.3 (M-t-Bu+H).

Tert-butyl (6R,7S)-7-cyano-6-phenyl-4-azaspiro[2.4]heptane-4-carboxylate or tert-butyl (6S,7R)-7-cyano-6-phenyl-4-azaspiro[2.4]heptane-4-carboxylate (D4.1) and tert-butyl (6R,7S)-7-cyano-6-phenyl-4-azaspiro[2.4]heptane-4-carboxylate or tert-butyl (6S,7R)-7-cyano-6-phenyl-4-azaspiro[2.4]heptane-4-carboxylate (D4.2). 14 g of Rac-tert-butyl (6S,7R)-7-cyano-6-phenyl-4-azaspiro[2.4]heptane-4-carboxylate was purified by chiral SFC (System: Waters SFC 150, Method: Column: DAICELCHIRALPAK®IG; Column size: 250*40 mm 10 μm; Injection: 2 mL; Mobile phase: MEOH (+0.1% 7.0 mol/1 Ammonia in MEOH); Flow rate: 80 ml/min; Wave length: UV 214 nm; Temperature: RT) to afford D4.1 (peak 1) and D4.2 (peak 2).

(6R,7S)-6-phenyl-4-azaspiro[2.4]heptane-7-carbonitrile or (6S,7R)-6-phenyl-4-azaspiro[2.4]heptane-7-carbonitrile (D4.3). A stirred solution of D4.1 (150 mg) in DCM (3 mL) was treated with TFA (0.6 mL) at room temperature. After 1 hour the mixture was concentrated to give the title compound, which was used directly without further purification. ES/MS m/z: (M+1) 177.2

### Preparation of (6R)-2,2-difluoro-6-phenyl-morpholine (D5.1)

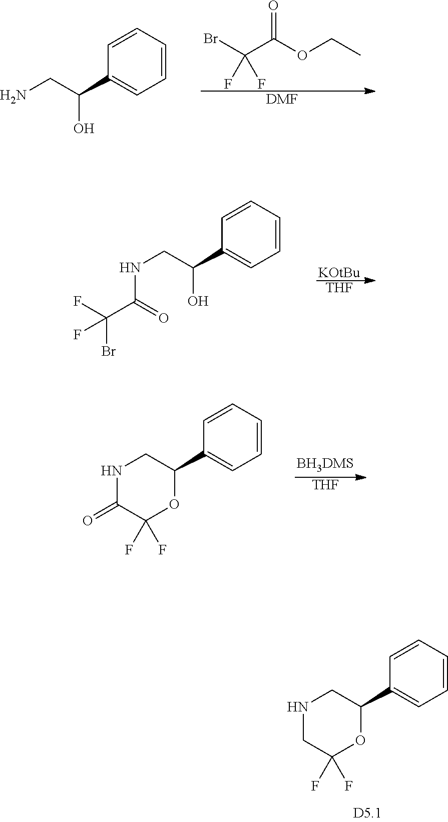

2-Bromo-2,2-difluoro-N-[(2R)-2-hydroxy-2-phenyl-ethyl]acetamide. To (1R)-2-amino-1-phenyl-ethanol (1.45 g, 1 eq)) in DMF (15 mL) was added ethyl 2-bromo-2,2-difluoroacetate (2.2 g, 1.05 eq). The reaction was stir overnight and then quenched by water. The mixture was extracted with EA and washed by brine. Purify the crude with normal phase column to yield 2-bromo-2,2-difluoro-N-[(2R)-2-hydroxy-2-phenyl-ethyl]acetamide. MS (ESI): m/z=293.4/295.6 [M+H]⁺

(6R)-2,2-difluoro-6-phenyl-morpholin-3-one. To a solution of 2-bromo-2,2-difluoro-N-[(2R)-2-hydroxy-2-phenyl-ethyl]acetamide (2.4 g, 1 eq) in THF (60 mL) was added a solution of potassium tertbutoxide in THF (I M in THF, 16 mL, 2 eq) dropwise over a period of 30 min at room temperature under nitrogen atmosphere. The mixture was heated to 70° C. for 10 min. After cooling to room temperature, water (100 mL) was added, the organic layer was separated, and the aqueous layer was extracted with ethyl acetate twice. The combined organic layer was washed with brine, dried over anhydrous sodium sulfate, filtered and concentrated. The residue was purified by flash chromatography to give the title compound. MS (ESI): m/z=214.0 [M+H]⁺

(6R)-2,2-difluoro-6-phenyl-morpholine. To (6R)-2,2-difluoro-6-phenyl-morpholin-3-one (981 mg, 1 eq) in THF (40 mL) was added borane dimethyl sulfide complex (1.3 mL, 3 eq) and stir overnight. The reaction mixture was quenched by 4N HCl in dioxanes and stir at 40° C. for 2 hours. The solution was directly concentrated down to yield (6R)-2,2-difluoro-6-phenyl-morpholine. MS (ESI): m/z=199.9 [M+H]⁺

### Preparation of ((2S,3R)-1-((S)-2-(5-((diethoxyphosphoryl)difluoromethyl)benzo[b]thiophene-2-carboxamido)-3,3-dimethylbutanoyl)-2-((R)-2-phenylmorpholine-4-carbonyl)pyrrolidin-3-yl)glycine (E1.2)

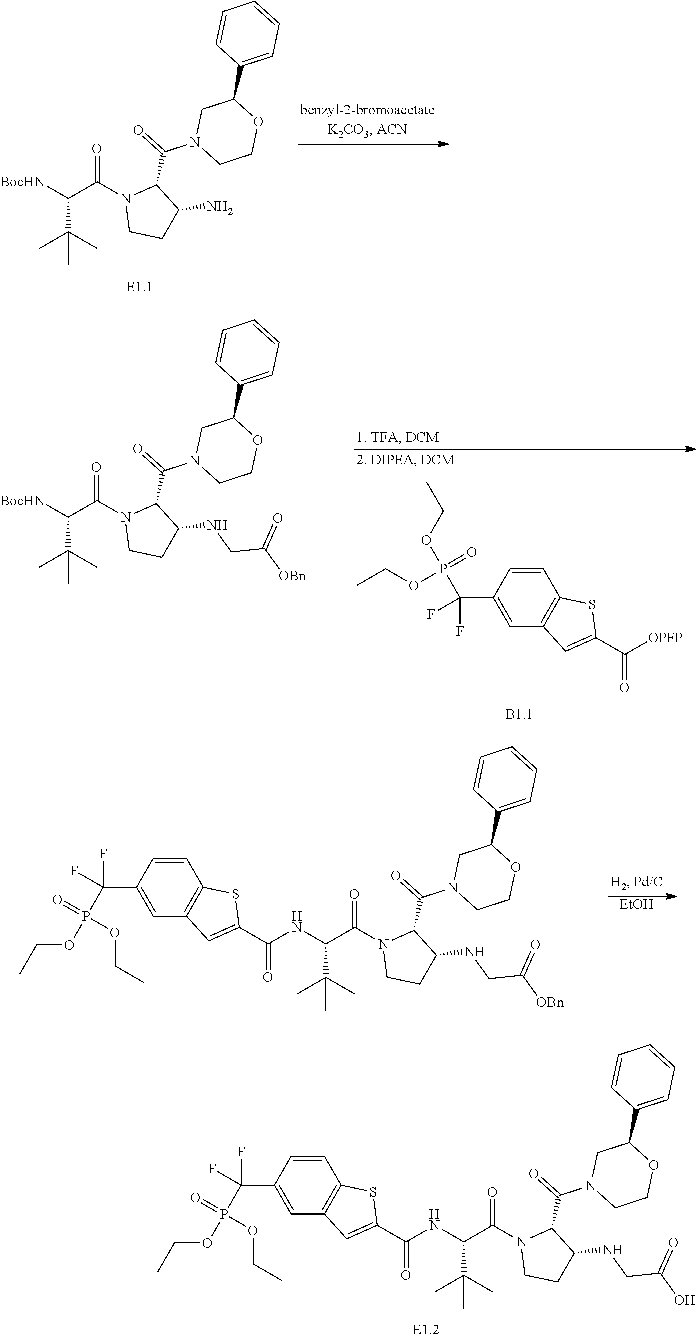

Benzyl ((2S,3R)-1-((S)-2-((tert-butoxycarbonyl)amino)-3,3-dimethylbutanoyl)-2-((R)-2-phenylmorpholine-4-carbonyl)pyrrolidin-3-yl)glycinate. To a suspension of tert-butyl ((S)-1-((2S,3R)-3-amino-2-((R)-2-phenylmorpholine-4-carbonyl)pyrrolidin-1-yl)-3,3-dimethyl-1-oxobutan-2-yl)carbamate (E1.1, 100 mg, 0.205 mmol) and potassium carbonate (113 mg, 0.819 mmol) in ACN (2 mL), was added benzyl 2-bromoacetate (0.0391 mL, 0.246 mmol). The reaction was stirred at rt overnight. The reaction mixture was diluted with EtOAc, washed with brine, and dried over sodium sulfate. The crude product was purified by silica gel column chromatography eluting with EtOAc and hexane to afford the title compound. ES/MS: m/z=637.2 [M]⁺.

benzyl ((2S,3R)-1-((S)-2-(5-((diethoxyphosphoryl)difluoromethyl)benzo[b]thiophene-2-carboxamido)-3,3-dimethylbutanoyl)-2-((R)-2-phenylmorpholine-4-carbonyl)pyrrolidin-3-yl)glycinate. To a solution of benzyl ((2S,3R)-1-((S)-2-((tert-butoxycarbonyl)amino)-3,3-dimethylbutanoyl)-2-((R)-2-phenylmorpholine-4-carbonyl)pyrrolidin-3-yl)glycinate (99.50 mg, 0.1563 mmol) in DCM (2 mL), was added Trifluoroacetic acid (0.1196 mL, 1.563 mmol). The reaction was stirred at rt overnight, then concentrated to dryness. The crude mixture was then resuspended in DCM (3 mL), and DIPEA (0.279 mL, 1.57 mmol) was added followed by B1.1 (84.2 mg, 0.159 mmol). The reaction was stirred at rt overnight, then concentrated to dryness and purified by silica gel column chromatography eluting with EtOAc and hexane to afford the title compound. ES/MS: m/z=883.2 [M]⁺.

((2S,3R)-1-((S)-2-(5-((diethoxyphosphoryl)difluoromethyl)benzo[b]thiophene-2-carboxamido)-3,3-dimethylbutanoyl)-2-((R)-2-phenylmorpholine-4-carbonyl)pyrrolidin-3-yl)glycine (E1.2). A suspension of benzyl ((2S,3R)-1-((S)-2-(5-((diethoxyphosphoryl)difluoromethyl)benzo[b]thiophene-2-carboxamido)-3,3-dimethylbutanoyl)-2-((R)-2-phenylmorpholine-4-carbonyl)pyrrolidin-3-yl)glycinate (133 mg, 0.151 mmol) in EtOH (10 mL) was degassed with Ar and purged with vacuum 3×. Added Palladium on carbon (10%) (10.0%, 16.0 mg, 0.0151 mmol) and stirred at rt under an atmosphere of hydrogen for 6 hr. The reaction was filtered over celite and rinsed with EtOAc to give title product, which was used without further purification. ES/MS: m/z=793.2 [M]⁺.

### Preparation of diethyl ((2-(((S)-1-((2S,3R)-3-(azetidin-3-ylmethyl)-2-((R)-2-phenylmorpholine-4-carbonyl)pyrrolidin-1-yl)-3,3-dimethyl-1-oxobutan-2-yl)carbamoyl)benzo[b]thiophen-5-yl)difluoromethyl)phosphonate (E2.3)

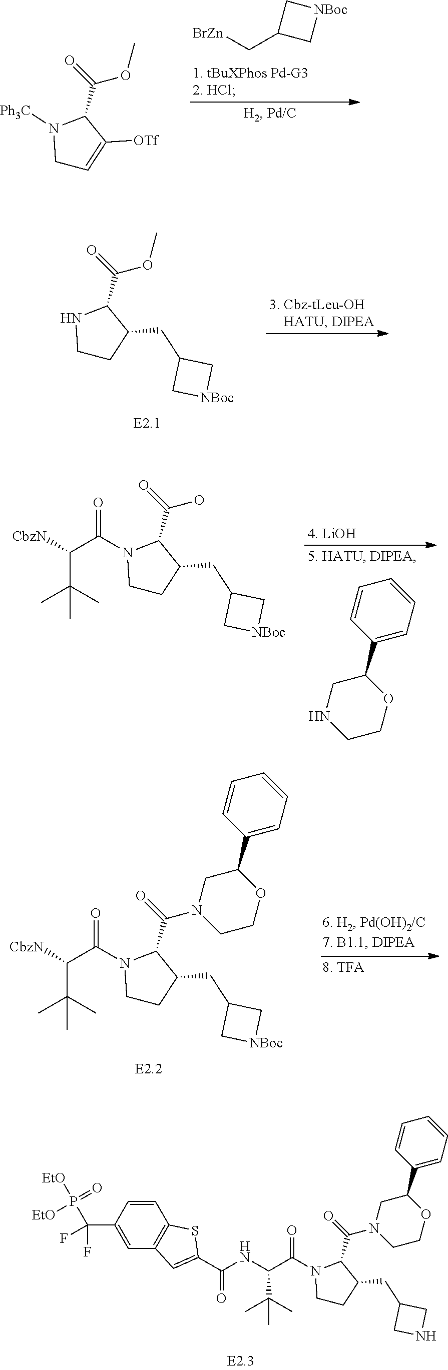

Step 1: A mixture of methyl (2S)-3-(trifluoromethylsulfonyloxy)-1-trityl-2,5-dihydropyrrole-2-carboxylate (170 mg, 0.33 mmol), (1-tert-butoxycarbonylazetidin-3-yl)methyl-iodo-zinc (0.5M, 3.3 mL, 1.6 mmol), and tBu-XPhos Pd-G3 (46 mg, 0.07 mmol) in THF (3.0 mL) were stirred at 55° C. under an argon atmosphere for 20 minutes. The mixture was cooled to room temperature, washed with sat. aq. NaHCO₃, and concentrated. Purification by flash column chromatography afforded methyl (2S)-3-[(1-tert-butoxycarbonylazetidin-3-yl)methyl]-1-trityl-2,5-dihydropyrrole-2-carboxylate. ES/MS: m/z=561.0 [M+Na]⁺. ¹H NMR (400 MHz, Methanol-d₄) δ 7.54-7.47 (m, 6H), 7.29-7.21 (m, 6H), 7.21-7.14 (m, 3H), 5.08 (s, 1H), 4.42 (d, J=4.7 Hz, 1H), 4.17-4.07 (m, 2H), 3.78 (t, J=8.4 Hz, 2H), 3.72 (s, 3H), 3.65-3.58 (m, 1H), 3.22-3.09 (m, 3H), 2.43-2.27 (m, 2H), 2.17-2.05 (m, 1H), 1.95-1.85 (m, 1H), 1.43 (s, 9H).

Step 2. To a solution of methyl (2S)-3-[(1-tert-butoxycarbonylazetidin-3-yl)methyl]-1-trityl-2,5-dihydropyrrole-2-carboxylate (110 mg, 0.20 mmol) in MeOH (4.0 mL) was added HCl (4.0M in dioxane, 0.05 mL, 0.20 mmol) at 0° C. After stirring for 30 minutes, the mixture was concentrated, then suspended in MeOH (4.0 mL). Pd/C (10% w/w, 65 mg, 0.06 mmol) was then added and the mixture was placed under a hydrogen atmosphere for 2 hours. The mixture was then filtered over Celite and concentrated to afford methyl (2S,3R)-3-[(1-tert-butoxycarbonylazetidin-3-yl)methyl]pyrrolidine-2-carboxylate (E2.1). ES/MS: m/z=243.0 [MtBu+H]⁺. ¹H NMR (400 MHz, Methanol-d₄) δ 4.44 (d, J=7.9 Hz, 1H), 4.16-3.98 (m, 2H), 3.89 (s, 3H), 3.64-3.50 (m, 4H), 2.74-2.13 (m, 4H), 1.88-1.48 (m, 2H), 1.43 (s, 9H).

Step 3. A solution of methyl (2S,3R)-3-[(1-tert-butoxycarbonylazetidin-3-yl)methyl]pyrrolidine-2-carboxylate (61 mg, 0.20 mmol), (2S)-2-(benzyloxycarbonylamino)-3,3-dimethyl-butanoic acid (65 mg, 0.25 mmol), HATU (93 mg, 0.25 mmol), and DIPEA (0.18 mL, 1.0 mmol) in MeCN (2.0 mL). After 1 hour, the mixture was concentrated and purified by flash column chromatography to afford methyl (2S,3R)-1-[(2S)-2-(benzyloxycarbonylamino)-3,3-dimethyl-butanoyl]-3-[(1-tert-butoxycarbonylazetidin-3-yl)methyl]pyrrolidine-2-carboxylate. ES/MS: m/z=568.1 [M+Na]⁺.

Step 4-5. LiOH (6.1 mg, 0.26 mmol) was added to a solution of methyl (2S,3R)-1-[(2S)-2-(benzyloxycarbonylamino)-3,3-dimethyl-butanoyl]-3-[(1-tert-butoxycarbonylazetidin-3-yl)methyl]pyrrolidine-2-carboxylate (70 mg, 0.13 mmol) in THF/MeOH/H₂O (1:1:1, 1.5 mL). After stirring for 3 hours, the mixture was brought to pH 5 with 1N HCl, then diluted with EtOAc. The organic layer was concentrated. The resulting residue was suspended in MeCN and (2R)-2-phenylmorpholine (25 mg, 0.15 mmol), HATU (59 mg, 0.15 mmol), and DIPEA (0.09 mL, 0.51 mmol) were added. After stirring for 1 hour, the mixture was concentrated and purified by flash column chromatography to afford tert-butyl 3-[[(2S,3R)-1-[(2S)-2-(benzyloxycarbonylamino)-3,3-dimethyl-butanoyl]-2-[(2R)-2-phenylmorpholine-4-carbonyl]pyrrolidin-3-yl]methyl]azetidine-1-carboxylate (E2.2). ES/MS: m/z=699.1 [M+Na]⁺.

Step 6. A mixture of tert-butyl 3-[[(2S,3R)-1-[(2S)-2-(benzyloxycarbonylamino)-3,3-dimethyl-butanoyl]-2-[(2R)-2-phenylmorpholine-4-carbonyl]pyrrolidin-3-yl]methyl]azetidine-1-carboxylate (86 mg, 0.13 mmol) and Pd(OH)2/C (20% w/w, 22 mg, 0.03 mmol) was stirred under an atmosphere of hydrogen overnight. The mixture was filtered over Celite and concentrated to afford tert-butyl 3-[[(2S,3R)-1-[(2S)-2-amino-3,3-dimethyl-butanoyl]-2-[(2R)-2-phenylmorpholine-4-carbonyl]pyrrolidin-3-yl]methyl]azetidine-1-carboxylate. ES/MS: m/z=543.2 [M+H]⁺.

Step 7. A mixture of tert-butyl 3-[[(2S,3R)-1-[(2S)-2-amino-3,3-dimethyl-butanoyl]-2-[(2R)-2-phenylmorpholine-4-carbonyl]pyrrolidin-3-yl]methyl]azetidine-1-carboxylate (69 mg, 0.13 mmol), (2,3,4,5,6-pentafluorophenyl) 5-[diethoxyphosphoryl(difluoro)methyl]benzothiophene-2-carboxylate B1.1 (67 mg, 0.13 mmol) and DIPEA (0.11 mL, 0.64 mmol) in DCM (2.0 mL) was stirred overnight. The mixture was concentrated and purified by flash column chromatography to afford tert-butyl 3-[[(2S,3R)-1-[(2S)-2-[[5-[diethoxyphosphoryl(difluoro)methyl]benzothiophene-2-carbonyl]amino]-3,3-dimethyl-butanoyl]-2-[(2R)-2-phenylmorpholine-4-carbonyl]pyrrolidin-3-yl]methyl]azetidine-1-carboxylate. ES/MS: m/z=887.1 [M−H]⁻.

Step 8. tert-butyl 3-[[(2S,3R)-1-[(2S)-2-[[5-[diethoxyphosphoryl(difluoro)methyl]benzothiophene-2-carbonyl]amino]-3,3-dimethyl-butanoyl]-2-[(2R)-2-phenylmorpholine-4-carbonyl]pyrrolidin-3-yl]methyl]azetidine-1-carboxylate was suspended in DCM/TFA (5:1, 1 mL). After stirring for 30 minutes, the mixture was concentrated to afford diethyl ((2-(((S)-1-((2S,3R)-3-(azetidin-3-ylmethyl)-2-((R)-2-phenylmorpholine-4-carbonyl)pyrrolidin-1-yl)-3,3-dimethyl-1-oxobutan-2-yl)carbamoyl)benzo[b]thiophen-5-yl)difluoromethyl)phosphonate (E2.3). ES/MS: m/z=789.2 [M+H]⁺.

### Preparation of 3-(5-(((R)-3-(((2S,3R)-1-((S)-2-amino-3,3-dimethylbutanoyl)-2-((R)-2-phenylmorpholine-4-carbonyl)pyrrolidin-3-yl)methyl)pyrrolidin-1-yl)methyl)-3-methyl-2-oxo-2,3-dihydro-1H-benzo[d]imidazol-1-yl)piperidine-2,6-dione (E2.5)

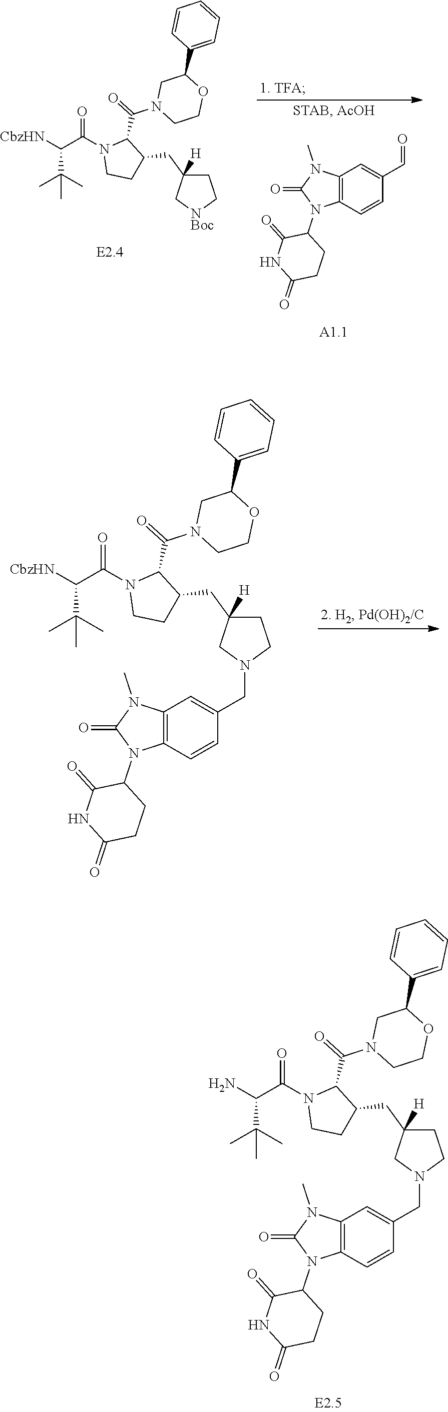 [SMILES: CC(C)(C)OC(=O)N1CC[C@@H](C[C@@H]2CCN(C(=O)[C@@H](NC(=O)OCc3ccccc3)C(C)(C)C)[C@@H]2C(=O)N2CCO[C@H](c3ccccc3)C2)C1 ; Cn1c(=O)n(C2CCC(=O)NC2=O)c2ccc(C=O)cc21 ; Cn1c(=O)n(C2CCC(=O)NC2=O)c2ccc(CN3CC[C@@H](C[C@@H]4CCN(C(=O)[C@@H](NC(=O)OCc5ccccc5)C(C)(C)C)[C@@H]4C(=O)N4CCO[C@H](c5ccccc5)C4)C3)cc21 ; Cn1c(=O)n(C2CCC(=O)NC2=O)c2ccc(CN3CC[C@@H](C[C@@H]4CCN(C(=O)[C@@H](N)C(C)(C)C)[C@@H]4C(=O)N4CCO[C@H](c5ccccc5)C4)C3)cc21]

Tert-butyl (R)-3-(((2S,3R)-1-((S)-2-(((benzyloxy)carbonyl)amino)-3,3-dimethylbutanoyl)-2-((R)-2-phenylmorpholine-4-carbonyl)pyrrolidin-3-yl)methyl)pyrrolidine-1-carboxylate (E2.4) was prepared in a similar manner to E2.2 starting with (S)-((1-(tert-butoxycarbonyl)pyrrolidin-3-yl)methyl)zinc(II) iodide. ES/MS: m/z=591.2 [M-Boc+H]⁺.

Step 1. To a solution of E2.4 (340 mg, 0.49 mmol) in DCM (8 mL) was added TFA (2 mL). After stirring for 1 hour, the mixture was concentrated, then suspended in EtOAc and washed with sat. aq. NaHCO₃. The organic layer was dried over MgSO4 and concentrated. The resulting residue was suspended in THF/DMF (3:1, 10 mL) and A1.1 (240 mg, 0.84 mmol) and AcOH (0.08 mL, 1.5 mmol) were added. After stirring for 1 hour, sodium triacetoxyborohydride (470 mg, 2.2 mmol) was added. After stirring overnight, the mixture was diluted with EtOAc and washed with sat. Aq. NaHCO₃. The organic layer was dried over MgSO4, filtered, and concentrated. Purification by reverse phase flash column chromatography afforded benzyl ((2S)-1-((3R)-3-(((3R)-1-((1-(2,6-dioxopiperidin-3-yl)-3-methyl-2-oxo-2,3-dihydro-1H-benzo[d]imidazol-5-yl)methyl)pyrrolidin-3-yl)methyl)-2-((R)-2-phenylmorpholine-4-carbonyl)pyrrolidin-1-yl)-3,3-dimethyl-1-oxobutan-2-yl)carbamate. ES/MS: m/z=862.4 [M+H]⁺.

Step 2. A suspension of benzyl ((2S)-1-((3R)-3-(((3R)-1-((1-(2,6-dioxopiperidin-3-yl)-3-methyl-2-oxo-2,3-dihydro-1H-benzo[d]imidazol-5-yl)methyl)pyrrolidin-3-yl)methyl)-2-((R)-2-phenylmorpholine-4-carbonyl)pyrrolidin-1-yl)-3,3-dimethyl-1-oxobutan-2-yl)carbamate (xx mg, xx mmol) and Pd(OH)2/C (xx mg, xx mmol) in THF (xx mL) was stirred under an atmosphere of hydrogen overnight. The mixture was filtered over Celite and concentrated to afford 3-(5-(((3R)-3-(((3R)-1-((S)-2-amino-3,3-dimethylbutanoyl)-2-((R)-2-phenylmorpholine-4-carbonyl)pyrrolidin-3-yl)methyl)pyrrolidin-1-yl)methyl)-3-methyl-2-oxo-2,3-dihydro-1H-benzo[d]imidazol-1-yl)piperidine-2,6-dione (E2.5). ES/MS: m/z=728.4 [M+H]⁺.

### Preparation of 3-(5-(((3S)-3-(((3R)-1-((S)-2-amino-3,3-dimethylbutanoyl)-2-((R)-2-phenylmorpholine-4-carbonyl)pyrrolidin-3-yl)methyl)pyrrolidin-1-yl)methyl)-3-methyl-2-oxo-2,3-dihydro-1H-benzo[d]imidazol-1-yl)piperidine-2,6-dione (E2.6)

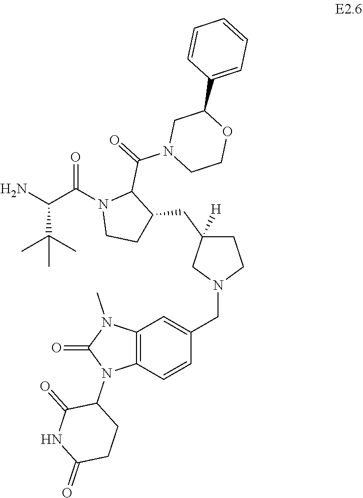 [SMILES: Cn1c(=O)n(C2CCC(=O)NC2=O)c2ccc(CN3CC[C@H](C[C@@H]4CCN(C(=O)[C@@H](N)C(C)(C)C)C4C(=O)N4CCO[C@H](c5ccccc5)C4)C3)cc21]

3-(5-(((3S)-3-(((3R)-1-((S)-2-amino-3,3-dimethylbutanoyl)-2-((R)-2-phenylmorpholine-4-carbonyl)pyrrolidin-3-yl)methyl)pyrrolidin-1-yl)methyl)-3-methyl-2-oxo-2,3-dihydro-1H-benzo[d]imidazol-1-yl)piperidine-2,6-dione (E2.6) was prepared in a similar manner to E2.5 starting with (R)-((1-(tert-butoxycarbonyl)pyrrolidin-3-yl)methyl)zinc(II) iodide. ES/MS: m/z=728.4 [M+H]⁺.

### Preparation of benzyl (2S,3S)-3-(1-(tert-butoxycarbonyl)azetidin-3-yl)-2-((R)-2-phenylmorpholine-4-carbonyl)pyrrolidine-1-carboxylate (E2.8)

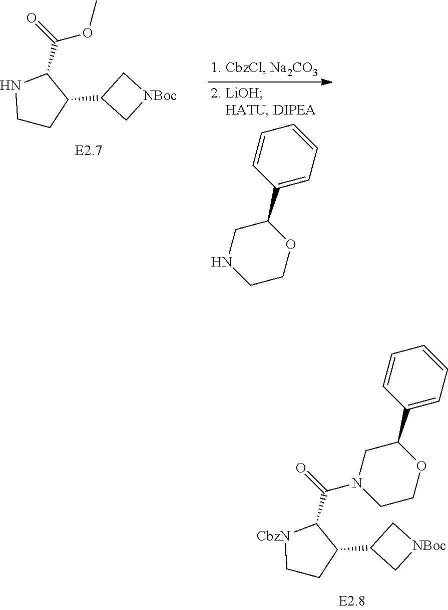 [SMILES: COC(=O)[C@H]1NCC[C@H]1C1CN(C(=O)OC(C)(C)C)C1 ; c1ccc([C@@H]2CNCCO2)cc1 ; CC(C)(C)OC(=O)N1CC([C@@H]2CCN(C(=O)OCc3ccccc3)[C@@H]2C(=O)N2CCO[C@H](c3ccccc3)C2)C1]

Methyl (2S,3S)-3-(1-(tert-butoxycarbonyl)azetidin-3-yl)pyrrolidine-2-carboxylate (E2.7) Was prepared in a similar manner to E2.1 starting with (1-(tert-butoxycarbonyl)azetidin-3-yl)zinc(II) iodide. ES/MS: m/z=285.1 [M+H]⁺.

Step 1. BnCOCl (0.21 mL, 1.44 mmol) was added to a stirred solution of E2.7 (341 mg, 1.2 mmol) and sodium carbonate (381 mg, 3.6 mmol) in Acetone/water (1:1 10 mL). The mixture was allowed to stir overnight, then diluted with EtOAc. The organic layer was separated, dried over MgSO4, filtered, and concentrated. Purification by flash column chromatography afforded 1-benzyl 2-methyl (2S,3S)-3-(1-(tert-butoxycarbonyl)azetidin-3-yl)pyrrolidine-1,2-dicarboxylate. ES/MS: m/z=441.1 [M+Na]⁺.

Step 2. LiOH (47 mg, 1.94 mmol) was added to a solution of 1-benzyl 2-methyl (2S,3S)-3-(1-(tert-butoxycarbonyl)azetidin-3-yl)pyrrolidine-1,2-dicarboxylate (406 mg, 0.97 mmol) in THF/MeOH/water (1:1:1, 10 mL). After 2 hours, the mixture was brought to pH 5 with 1N HCl and diluted with EtOAc. The organic layer was separated and concentrated. The residue was suspended in MeCN and (2R)-2-phenylmorpholine (190 mg, 1.16 mmol), HATU (442 mg, 1.16 mmol), and DIPEA (0.69 mL, 3.88 mmol) were added. After stirring for 1 hour, the mixture was concentrated and purified by flash column chromatography to afford benzyl (2S,3S)-3-(1-(tertbutoxycarbonyl)azetidin-3-yl)-2-((R)-2-phenylmorpholine-4-carbonyl)pyrrolidine-1-carboxylate (E2.8) ES/MS: m/z=550.1 [M+H]⁺.

### Preparation of 3-(5-((3-((2S,3S)-1-((S)-2-amino-3,3-dimethylbutanoyl)-2-((R)-2-phenylmorpholine-4-carbonyl)pyrrolidin-3-yl)azetidin-1-yl)methyl)-3-methyl-2-oxo-2,3-dihydro-1H-benzo[d]imidazol-1-yl)piperidine-2,6-dione (E2.9)

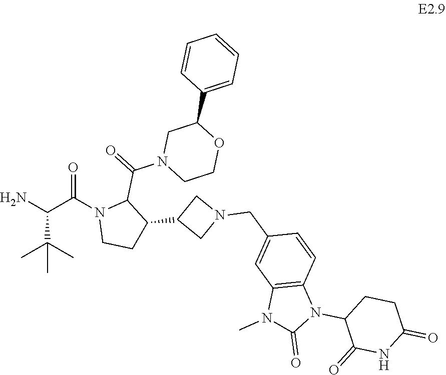 [SMILES: Cn1c(=O)n(C2CCC(=O)NC2=O)c2ccc(CN3CC([C@@H]4CCN(C(=O)[C@@H](N)C(C)(C)C)C4C(=O)N4CCO[C@H](c5ccccc5)C4)C3)cc21]

3-(5-((3-((2S,3S)-1-((S)-2-amino-3,3-dimethylbutanoyl)-2-((R)-2-phenylmorpholine-4-carbonyl)pyrrolidin-3-yl)azetidin-1-yl)methyl)-3-methyl-2-oxo-2,3-dihydro-1H-benzo[d]imidazol-1-yl)piperidine-2,6-dione (E2.9) was prepared in a similar manner to C5.3 starting with E2.8. ES/MS: m/z=700.2 [M+H]⁺.

### IV. Compounds

### Example 1.1: ((2-(((2S)-1-((2S,3R)-3-(2-(4-(1-(2,6-dioxopiperidin-3-yl)-3-methyl-2-oxo-2,3-dihydro-1H-benzo[d]imidazol-5-yl)piperidin-1-yl)-2-oxoethoxy)-2-((R)-2-phenylmorpholine-4-carbonyl)pyrrolidin-1-yl)-3,3-dimethyl-1-oxobutan-2-yl)carbamoyl)benzo[b]thiophen-5-yl)difluoromethyl)phosphonic acid

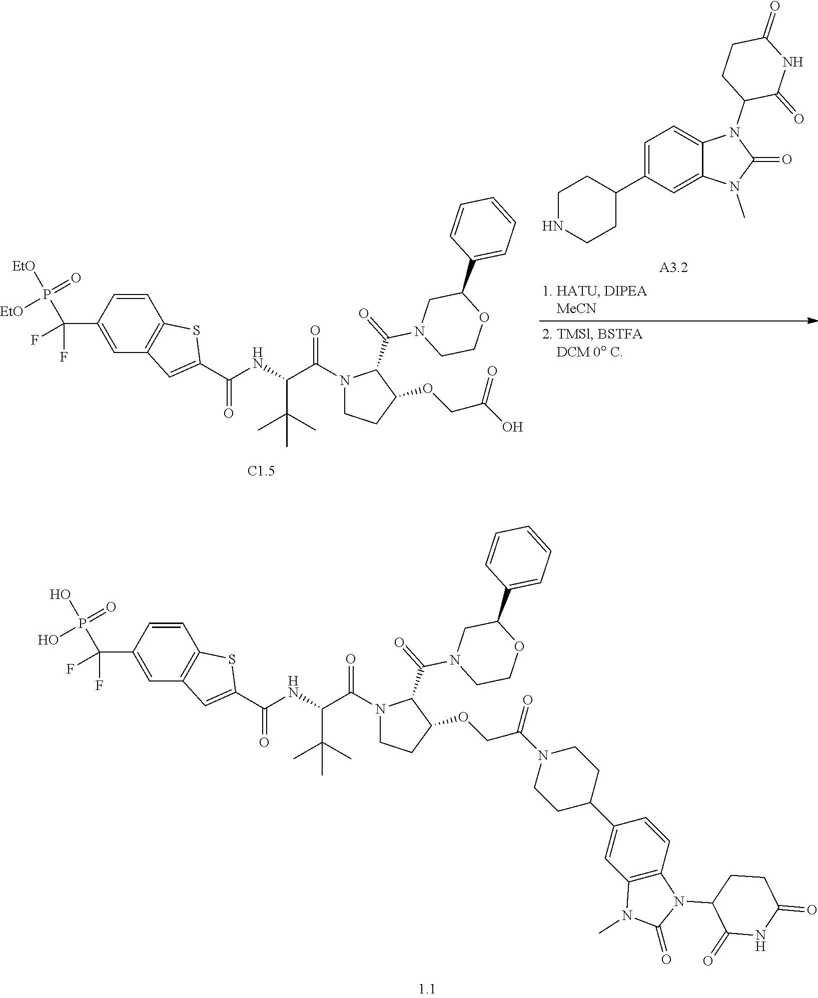

To a mixture of 2-(((2S,3R)-1-((S)-2-(5-((diethoxyphosphoryl)difluoromethyl)benzo[b]thiophene-2-carboxamido)-3,3-dimethylbutanoyl)-2-((R)-2-phenylmorpholine-4-carbonyl)pyrrolidin-3-yl)oxy)acetic acid (40 mg, 0.05 mmol) C1.5 and 3-(3-methyl-2-oxo-5-(piperidin-4-yl)-2,3-dihydro-1H-benzo[d]imidazol-1-yl)piperidine-2,6-dione (21 mg, 0.06 mmol) A3.2 in MeCN (3 mL) was added HATU (18 mg, 0.05 mmol) and DIPEA (0.45 mL, 0.25 mmol). After stirring for 30 minutes, the mixture was concentrated and purified by silica gel flash column chromatography. The purified residue was resuspended in DCM and N,O-Bis(trimethylsilyl)trifluoroacetamide (0.12 mL, 0.45 mmol) and TMSI (1.0M in DCM, 0.15 mL, 0.15 mmol) were added at 0° C. After 30 minutes, the mixture was concentrated. Purification by reverse phase HPLC afforded ((2-(((2S)-1-((2S,3R)-3-(2-(4-(1-(2,6-dioxopiperidin-3-yl)-3-methyl-2-oxo-2,3-dihydro-1H-benzo[d]imidazol-5-yl)piperidin-1-yl)-2-oxoethoxy)-2-((R)-2-phenylmorpholine-4-carbonyl)pyrrolidin-1-yl)-3,3-dimethyl-1-oxobutan-2-yl)carbamoyl)benzo[b]thiophen-5-yl)difluoromethyl)phosphonic acid (1.1).

The compounds in Table 1.1 were prepared analogously to Example 1.1 modified as described in Table 1.2

TABLE 1.1
Examples 1.2 to 1.46

| Ex. | Name | Structure |
|---|---|---|
| 1.2 | ((2-(((2S)-1-((2S,3R)-3-(2- (8-((1-(2,6-dioxopiperidin- 3-yl)-3-methyl-2-oxo-2,3- dihydro-1H-benzo[d]imid- azol-5-yl)methyl)-3,8- diazabicyclo[3.2.1]octan- 3-yl)-2-oxoethoxy)-2-((R)- 2-phenylmorpholine-4- carbonyl)pyrrolidin-1-yl)- 3,3-dimethyl-1-oxobutan- 2-yl)carbamoyl)benzo[b] thiophen-5-yl)difluoro- methyl)phosphonic acid | 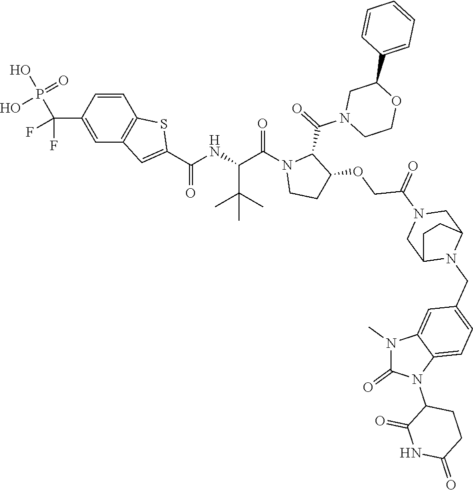 [SMILES: Cn1c(=O)n(C2CCC(=O)NC2=O)c2ccc(CN3C4CCC3CN(C(=O)CO[C@@H]3CCN(C(=O)[C@@H](NC(=O)c5cc6cc(C(F)(F)P(=O)(O)O)ccc6s5)C(C)(C)C)[C@@H]3C(=O)N3CCO[C@H](c5ccccc5)C3)C4)cc21] |
| 1.3 | 2-((2-(((2S)-1-((2S,3R)-3- (2-((((1r,4R)-4-((1-(2,6- dioxopiperidin-3-yl)-3- methyl-2-oxo-2,3-dihydro- 1H-benzo[d]imidazol-5- yl)methyl)cyclohexyl)- methyl)(methyl)amino)-2- oxoethoxy)-2-((R)-2-phen- ylmorpholine-4-carbonyl)- pyrrolidin-1-yl)-3,3- dimethyl-1-oxobutan-yl)- carbamoyl)benzo[b]thio- phen-5-yl)difluoromethyl)- phosphonic acid | 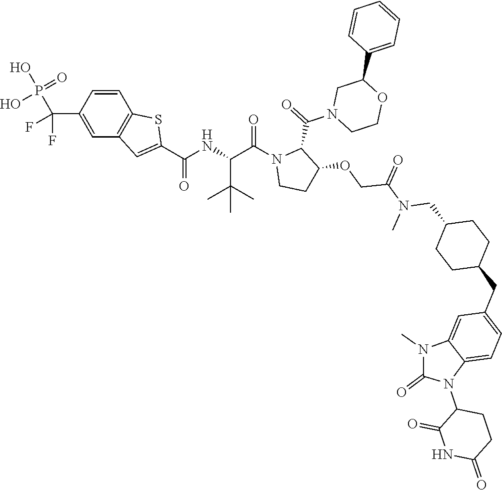 [SMILES: CN(C[C@H]1CC[C@H](Cc2ccc3c(c2)n(C)c(=O)n3C2CCC(=O)NC2=O)CC1)C(=O)CO[C@@H]1CCN(C(=O)[C@@H](NC(=O)c2cc3cc(C(F)(F)P(=O)(O)O)ccc3s2)C(C)(C)C)[C@@H]1C(=O)N1CCO[C@H](c2ccccc2)C1] |
| 1.4 | ((2-(((2S)-1-((2S,3R)-3-(2- (4-((1-(2,6-dioxopiperidin- 3-yl)-3-methyl-2-oxo-2,3- dihydro-1H-benzo[d]imid- azol-5-yl)methyl)piperidin- 1-yl)-2-oxoethoxy)-2-((R)- 2-phenylmorpholine-4- carbonyl)pyrrolidin-1-yl)- 3,3-dimethyl-1-oxobutan- 2-yl)carbamoyl)benzo[b] thiophen-5-yl)difluoro- methyl)phosphonic acid | 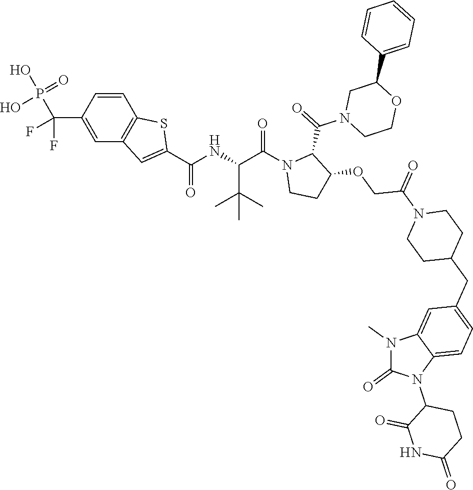 [SMILES: Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21] |
| 1.5 | ((2-(((2S)-1-((2S,3R)-3-(2- (4-(1-(2,6-dioxopiperidin- 3-yl)-3-methyl-2-oxo-2,3- dihydro-1H-benzo[d]imid- azol-5-yl)-[1,4′-bipiperi- din]-1′-yl)-2-oxoethoxy)-2- ((R)-2-phenylmorpholine- 4-carbonyl)pyrrolidin-1- yl)-3,3-dimethyl-1-oxobu- tan-2-yl)carbamoyl)benzo [b]thiophen-5-yl)difluoro- methyl)phosphonic acid | 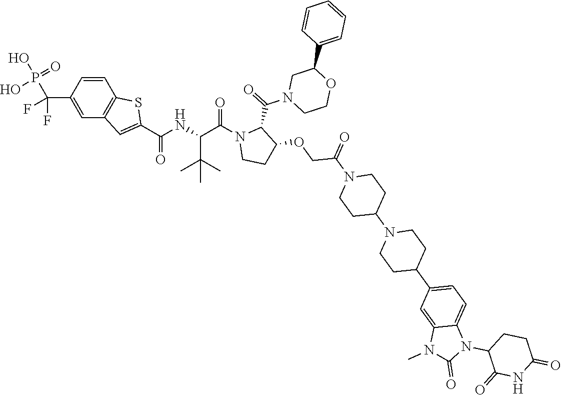 [SMILES: Cn1c(=O)n(C2CCC(=O)NC2=O)c2ccc(C3CCN(C4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21] |
| 1.6 | ((2-(((2S)-1-((2S,3R)-3-(2- (4-((1-(1-(2,6-dioxopiperi- din-3-yl)-3-methyl-2-oxo- 2,3-dihydro-1H-benzo[d] imidazol-5-yl)piperidin-4- yl)oxy)piperidin-1-yl)-2- oxoethoxy)-2-((R)-2-phen- ylmorpholine-4-carbonyl)- pyrrolidin-1-yl)-3,3-di- methyl-1-oxobutan-2-yl)- carbamoyl)benzo[b]thio- phen-5-yl)-difluorometh- yl)phosphonic acid | 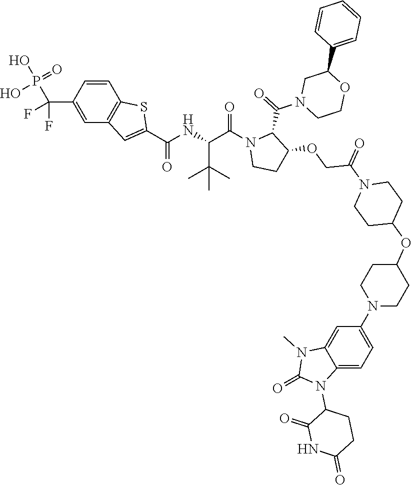 [SMILES: Cn1c(=O)n(C2CCC(=O)NC2=O)c2ccc(N3CCC(OC4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21] |
| 1.7 | ((2-(((2S)-1-((2S,3R)-3-(2- (1′-(1-(2,6-dioxopiperidin- 3-yl)-3-methyl-2-oxo-2,3- dihydro-1H-benzo[d]imid- azol-5-yl)-[4,4′-bipiperi- din]-1-yl)-2-oxoethoxy)-2- ((R)-2-phenylmorpholine- 4-carbonyl)pyrrolidin-1-yl)- 3,3-dimethyl-1-oxobutan- 2-yl)carbamoyl)benzo[b] thiophen-5-yl)difluoro- methyl)phosphonic acid | 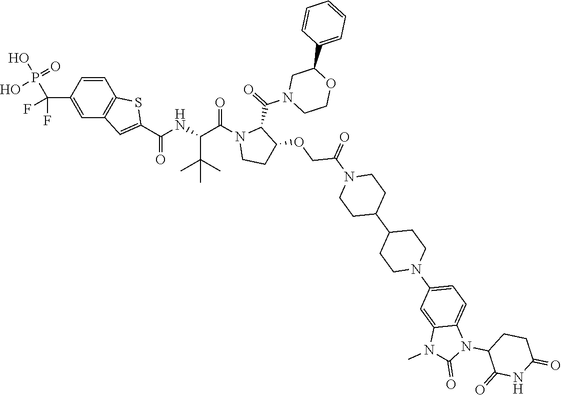 [SMILES: Cn1c(=O)n(C2CCC(=O)NC2=O)c2ccc(N3CCC(C4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21] |
| 1.8 | ((2-(((2S)-1-((2S,3R)-3-(2- (4-((1-(1-(2,6-dioxopiperi- din-3-yl)-3-methyl-2-oxo- 2,3-dihydro-1H-benzo[d] imidazol-5-yl)piperidin-4- yl)methyl)piperidin-1-yl)- 2-oxoethoxy)-2-((R)-2- phenylmorpholine-4- carbonyl)pyrrolidin-1-yl)- 3,3-dimethyl-1-oxobutan- 2-yl)carbamoyl)benzo[b] thiophen-5-yl)difluoro- methyl)phosphonic acid | 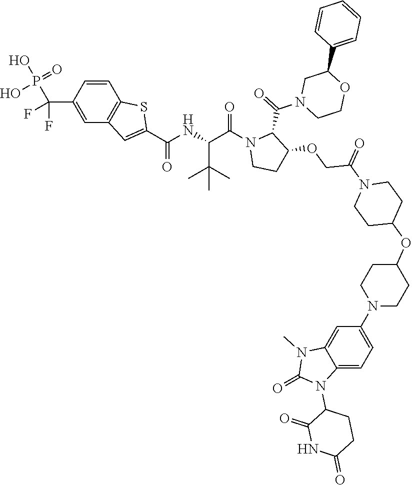 [SMILES: Cn1c(=O)n(C2CCC(=O)NC2=O)c2ccc(N3CCC(OC4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21] |
| 1.9 | ((2-(((2S)-1-((2S,3R)-3-((1- (1-(2,6-dioxopiperidin-3- yl)-3-methyl-2-oxo-2,3- dihydro-1H-benzo[d]imid- azole-5-carbonyl)piperidin- 4-yl)methyl)-2-((R)-2- phenylmorpholine-4- carbonyl)pyrrolidin-1-yl)- 3,3-dimethyl-1-oxobutan- 2-yl)carbamoyl)benzo[b] thiophen-5-yl)difluoro- methyl)phosphonic acid | 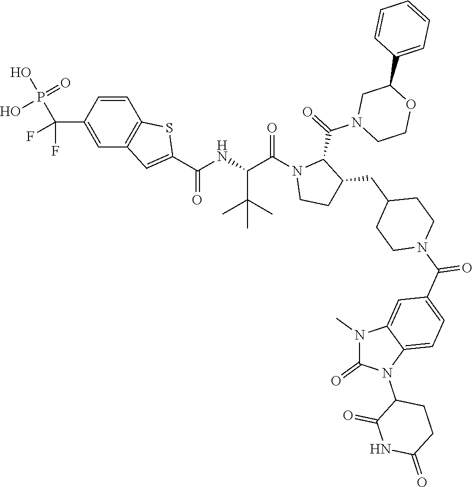 [SMILES: Cn1c(=O)n(C2CCC(=O)NC2=O)c2ccc(C(=O)N3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21] |
| 1.10 | ((2-(((2S)-1-((2S,3R)-3-(2- (4-(1-(2,6-dioxopiperidin- 3-yl)-3-methyl-2-oxo-2,3- dihydro-1H-benzo[d]imid- azol-5-yl)piperazin-1-yl)-2- oxoethoxy)-2-((R)-2-phen- ylmorpholine-4-carbonyl)- pyrrolidin-1-yl)-3,3-di- methyl-1-oxobutan-2-yl)- carbamoyl)benzo[b]thio- phen-5-yl)difluoromethyl)- phosphonic acid | 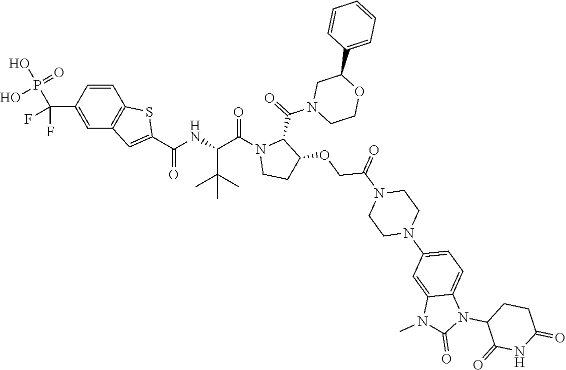 [SMILES: Cn1c(=O)n(C2CCC(=O)NC2=O)c2ccc(N3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21] |
| 1.11 | ((2-(((2S)-1-((2S,3R)-3-(2- (4-(1-(1-(2,6-dioxopiperi- din-3-yl)-3-methyl-2-oxo- 2,3-dihydro-1H-benzo[d] imidazol-5-yl)piperidin-4- yl)piperazin-1-yl)-2-oxo- ethoxy)-2-((R)-2-phenyl- morpholine-4-carbonyl)- pyrrolidin-1-yl)-3,3-di- methyl-1-oxobutan-2-yl)- carbamoyl)benzo[b]thio- phen-5-yl)difluoromethyl)- phosphonic acid | 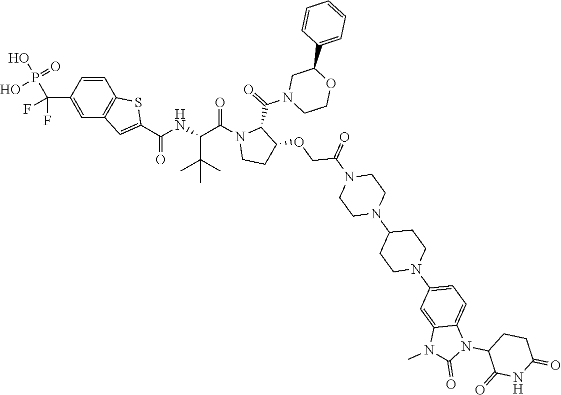 [SMILES: Cn1c(=O)n(C2CCC(=O)NC2=O)c2ccc(N3CCC(N4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21] |
| 1.12 | ((2-(((2S)-1-((2S,3R)-3-(2- (4-(1-(2,6-dioxopiperidin- 3-yl)-3-methyl-2-oxo-2,3- dihydro-1H-benzo[d]imid- azole-5-carbonyl)piperazin- 1-yl)-2-oxoethoxy)-2-((R)- 2-phenylmorpholine-4- carbonyl)pyrrolidin-1-yl)- 3,3-dimethyl-1-oxobutan- 2-yl)carbamoyl)benzo[b] thiophen-5-yl)difluoro- methyl)phosphonic acid | 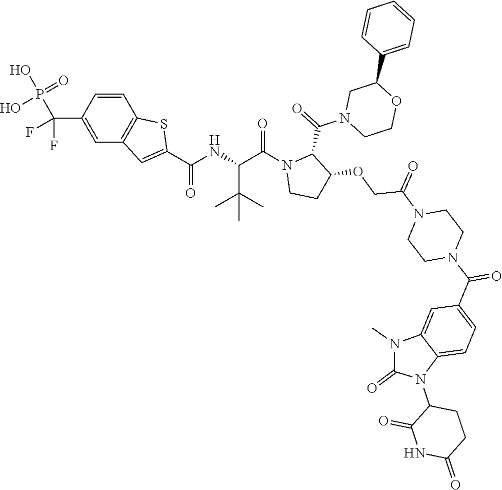 [SMILES: Cn1c(=O)n(C2CCC(=O)NC2=O)c2ccc(C(=O)N3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21] |
| 1.13 | ((2-(((2S)-1-((2S,3R)-3-(2- (8-(1-(2,6-dioxopiperidin- 3-yl)-3-methyl-2-oxo-2,3- dihydro-1H-benzo[d]imid- azol-5-yl)-3,8-diazabicyclo [3.2.1]octan-3-yl)-2-oxo- ethoxy)-2-((R)-2-phenyl- morpholine-4-carbonyl)- pyrrolidin-1-yl)-3,3-di- methyl-1-oxobutan-2-yl)- carbamoyl)benzo[b]thio- phen-5-yl)difluoromethyl)- phosphonic acid | 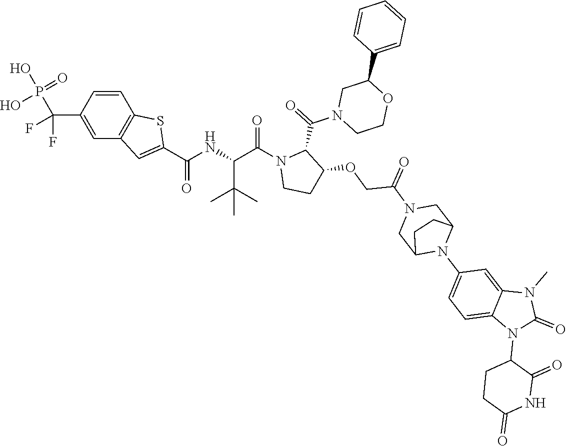 [SMILES: Cn1c(=O)n(C2CCC(=O)NC2=O)c2ccc(N3C4CCC3CN(C(=O)CO[C@@H]3CCN(C(=O)[C@@H](NC(=O)c5cc6cc(C(F)(F)P(=O)(O)O)ccc6s5)C(C)(C)C)[C@@H]3C(=O)N3CCO[C@H](c5ccccc5)C3)C4)cc21] |
| 1.14 | ((2-(((2S)-1-((2S,3R)-3-(2- ((((1r,4R)-4-((1-(2,6-dioxo- piperidin-3-yl)-3-methyl-2- oxo-2,3-dihydro-1H-benzo [d]imidazol-5-yl)methyl)- cyclohexyl)methyl)amino)- 2-oxoethoxy)-2-((R)-2- phenylmorpholine-4- carbonyl)pyrrolidin-1-yl)- 3,3-dimethyl-1-oxobutan- 2-yl)carbamoyl)benzo[b] thiophen-5-yl)difluoro- methyl)-phosphonic acid | 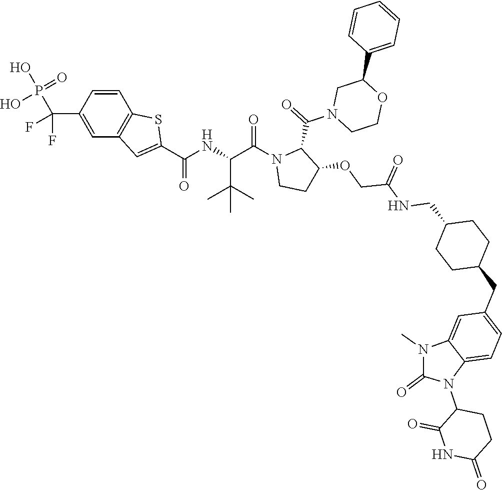 [SMILES: Cn1c(=O)n(C2CCC(=O)NC2=O)c2ccc(C[C@H]3CC[C@H](CNC(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21] |
| 1.15 | ((2-(((2S)-1-((2S,3R)-3-(2- (9-(1-(2,6-dioxopiperidin- 3-yl)-3-methyl-2-oxo-2,3- dihydro-1H-benzo[d]imid- azol-5-yl)-3,9-diazaspiro [5.5]undecan-3-yl)-2-oxo- ethoxy)-2-((R)-2-phenyl- morpholine-4-carbonyl)- pyrrolidin-1-yl)-3,3-di- methyl-1-oxobutan-2-yl)- carbamoyl)benzo[b]thio- phen-5-yl)difluoromethyl)- phosphonic acid | 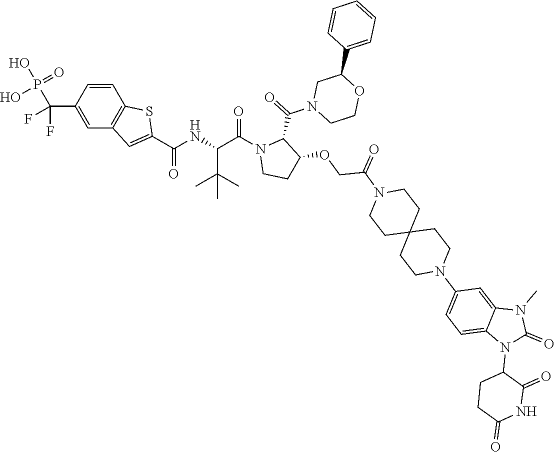 [SMILES: Cn1c(=O)n(C2CCC(=O)NC2=O)c2ccc(N3CCC4(CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21] |
| 1.16 | (2-(4-((1-(2,6-dioxopiperi- din-3-yl)-3-methyl-2-oxo- 2,3-dihydro-1H-benzo[d] imidazol-5-yl)methyl)- piperazin-1-yl)-2-oxoeth- oxy)-2-((R)-2-phenylmor- pho-line-4-carbonyl)pyr- rolidin-1-yl)-3,3-dimethyl- 1-oxobutan-2-yl)carbamo- yl)benzo[b]thiophen-5-yl)- difluoromethyl)phosphonic acid | 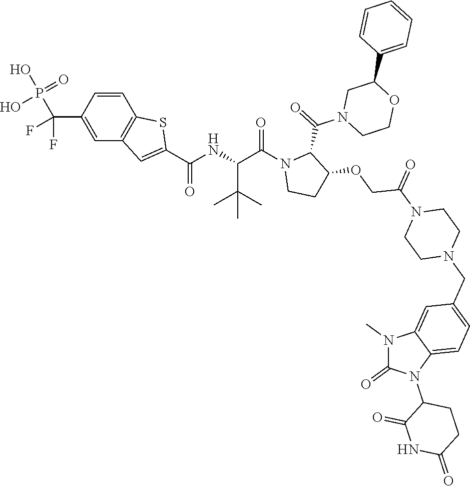 [SMILES: Cn1c(=O)n(C2CCC(=O)NC2=O)c2ccc(CN3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21] |
| 1.17 | ((2-(((2S)-1-((2S,3R)-3-(2- (4-((1-(2,6-dioxopiperidin- 3-yl)-3-methyl-2-oxo-2,3- dihydro-1H-benzo[d]imid- azol-5-yl)methyl)piperidin- 1-yl)-2-oxoethoxy)-2-(6- (pyridin-3-yl)-4-azaspiro [2.4]heptane-4-carbonyl)- pyrrolidin-1-yl)-3,3-di- methyl-1-oxobutan-2-yl)- carbamoyl)benzo[b]thio- phen-5-yl)difluoromethyl)- phosphonic acid | 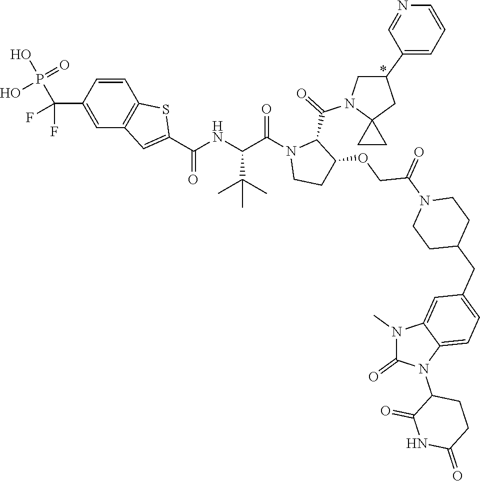 [SMILES: Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CC(c5cccnc5)CC45CC5)CC3)cc21] |
| 1.18 | ((2-(((2S)-1-((2S,3R)-3-(2- (4-((1-(2,6-dioxopiperidin- 3-yl)-3-methyl-2-oxo-2,3- dihydro-1H-benzo[d]imid- azol-5-yl)methyl)piperidin- 1-yl)-2-oxoethoxy)-2-(6- (pyridin-3-yl)-4-azaspiro [2.4]heptane-4-carbonyl)- pyrrolidin-1-yl)-3,3-di- methyl-1-oxobutan-2-yl)- carbamoyl)benzo[b]thio- phen-5-yl)difluoromethyl)- phosphonic acid | 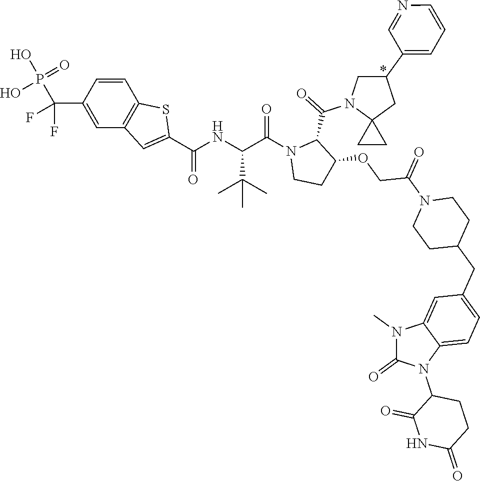 [SMILES: Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CC(c5cccnc5)CC45CC5)CC3)cc21] |
| 1.19 | ((2-(((2S)-1-((2S,3R)-2- ((6S,7R)-7-cyano-6-phen- yl-4-azaspiro[2.4]heptane- 4-carbonyl)-3-(2-(4-((1- (2,6-dioxopiperidin-3-yl)- 3-methyl-2-oxo-2,3-di- hydro-1H-benzo[d]imid- azol-5-yl)methyl)piperidin- 1-yl)-2-oxoethoxy)pyrroli- din-1-yl)-3,3-dimethyl-1- oxobutan-2-yl)carbamoyl)- benzo[b]thiophen-5-yl)- difluoromethyl)phosphonic acid or ((2-(((2S)-1-((2S, 3R)-2-((6R,7S)-7-cyano-6- phenyl-4-azaspiro[2.4] heptane-4-carbonyl)-3-(2- (4-((1-(2,6-dioxopiperidin- 3-yl)-3-methyl-2-oxo-2,3- dihydro-1H-benzo[d]imid- azol-5-yl)methyl)piperidin- 1-yl)-2-oxoethoxy)pyrroli- din-1-yl)-3,3-dimethyl-1- oxobutan-2-yl)carbamoyl)- benzo[b]thiophen-5-yl)- difluoromethyl)phosphonic acid | 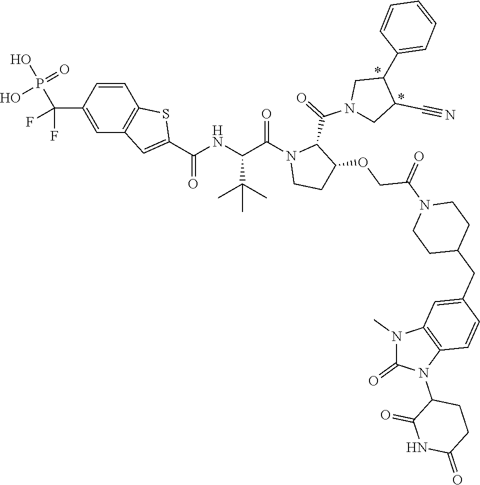 [SMILES: Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CC(C#N)C(c5ccccc5)C4)CC3)cc21] |
| 1.20 | ((2-(((2S)-1-((2S,3R)-3-(2- (4-((1-(2,6-dioxopiperidin- 3-yl)-6-fluoro-3-methyl-2- oxo-2,3-dihydro-1H-benzo [d]imidazol-5-yl)methyl)- piperidin-1-yl)-2-oxo- ethoxy)-2-((R)-2-phenyl- morpholine-4-carbonyl)- pyrrolidin-1-yl)-3,3-di- methyl-1-oxobutan-2-yl)- carbamoyl)benzo[b]thio- phen-5-yl)difluoromethyl)- phosphonic acid | 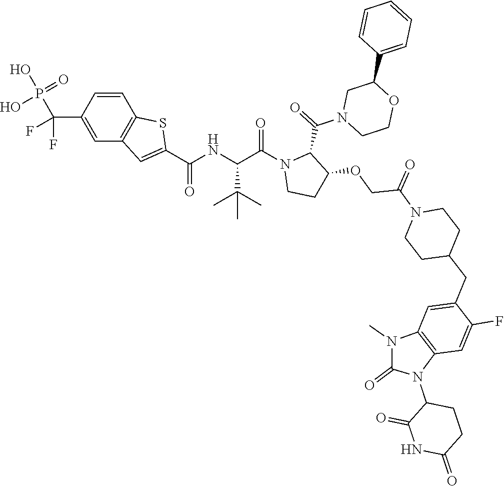 [SMILES: Cn1c(=O)n(C2CCC(=O)NC2=O)c2cc(F)c(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21] |
| 1.21 | ((2-(((2S)-1-((2S,3R)-2- ((R)-2,2-difluoro-6-phen- ylmorpholine-4-carbonyl)- 3-(2-(4-((1-(2,6-dioxo- piperidin-3-yl)-3-methyl-2- oxo-2,3-dihydro-1H-benzo [d]imidazol-5-yl)methyl)- piperidin-1-yl)-2-oxoeth- oxy)pyrrolidin-1-yl)-3,3- dimethyl-1-oxobutan-2-yl)- carbamoyl)benzo[b]thio- phen-5-yl)difluoromethyl)- phosphonic acid | 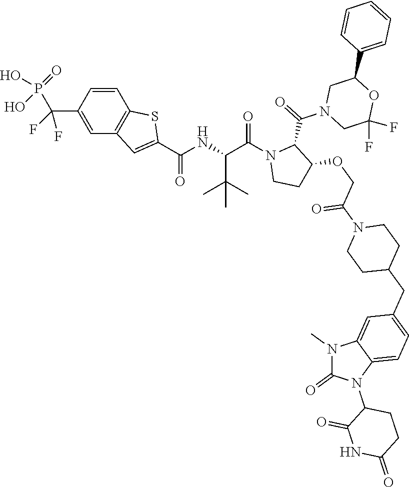 [SMILES: Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4C[C@@H](c5ccccc5)OC(F)(F)C4)CC3)cc21] |
| 1.22 | ((2-(((2S)-1-((2S,3R)-3-(2- (4-((1-(2,6-dioxopiperidin- 3-yl)-3-methyl-2-oxo-2,3- dihydro-1H-benzo[d]imid- azol-5-yl)methyl)piperidin- 1-yl)-2-oxoethoxy)-2-((R)- 4-methyl-3-phenylpipera- zine-1-carbonyl)pyrrolidin- 1-yl)-3,3-dimethyl-1-oxo- butan-2-yl)carbamoyl)- benzo[b]thiophen-5-yl)di- fluoromethyl)phosphonic acid | 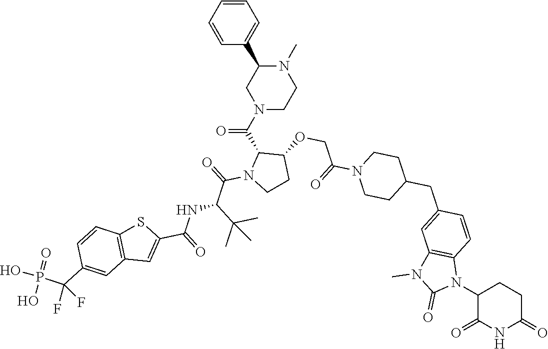 [SMILES: CN1CCN(C(=O)[C@@H]2[C@H](OCC(=O)N3CCC(Cc4ccc5c(c4)n(C)c(=O)n5C4CCC(=O)NC4=O)CC3)CCN2C(=O)[C@@H](NC(=O)c2cc3cc(C(F)(F)P(=O)(O)O)ccc3s2)C(C)(C)C)C[C@H]1c1ccccc1] |
| 1.23 | ((2-(((2S)-1-((2S,3R)-3-(2- (4-((1-(2,6-dioxopiperidin- 3-yl)-4-fluoro-3-methyl-2- oxo-2,3-dihydro-1H-benzo [d]imidazol-5-yl)methyl)- piperidin-1-yl)-2-oxoeth- oxy)-2-((R)-2-phenylmor- pholine-4-carbonyl)pyrroli- din-1-yl)-3,3-dimethyl-1- oxobutan-2-yl)carbamoyl)- benzo[b]thiophen-5-yl)- difluoromethyl)phosphonic acid | 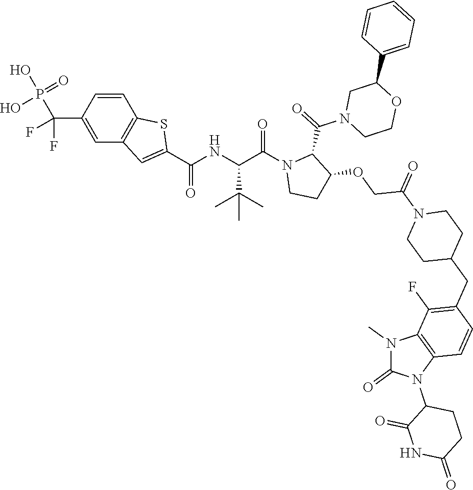 [SMILES: Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)c(F)c21] |
| 1.24 | ((1S)-(2-(((2S)-1-((2S,3R)- 3-(2-(4-((1-(2,6-dioxopiper- idin-3-yl)-3-methyl-2-oxo- 2,3-dihydro-1H-benzo[d] imidazol-5-yl)methyl)piper- azin-1-yl)-2-oxoethoxy)-2- ((R)-2-phenylmorpholine- 4-carbonyl)pyrrolidin-1-yl)- 3,3-dimethyl-1-oxobutan- 2-yl)carbamoyl)-4-fluoro- benzo[b]thiophen-5-yl)- fluoromethyl)phosphonic acid or ((1R)-(2-(((2S)-1- ((2S,3R)-3-(2-(4-((1-(2,6- dioxopiperidin-3-yl)-3- methyl-2-oxo-2,3-dihydro- 1H-benzo[d]imidazol-5- yl)methyl)piperazin-1-yl)- 2-oxoethoxy)-2-((R)-2- phenylmorpholine-4- carbonyl)pyrrolidin-1-yl)- 3,3-dimethyl-1-oxobutan- 2-yl)carbamoyl)-4-fluoro- benzo[b]thiophen-5-yl)- fluoromethyl)phosphonic acid | 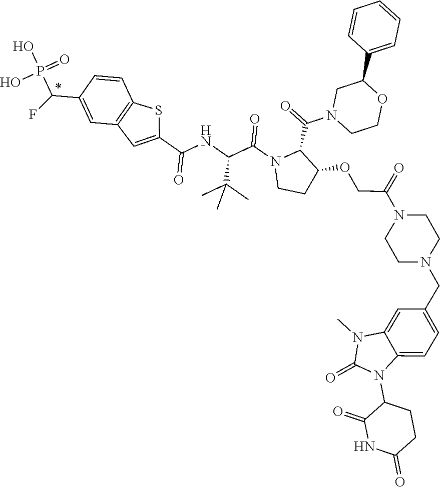 [SMILES: Cn1c(=O)n(C2CCC(=O)NC2=O)c2ccc(CN3CCN(C(=O)CO[C@@H]4CCN(C(=O)[C@@H](NC(=O)c5cc6cc(C(F)P(=O)(O)O)ccc6s5)C(C)(C)C)[C@@H]4C(=O)N4CCO[C@H](c5ccccc5)C4)CC3)cc21] |
| 1.25 | ((2-(((2S)-1-((2S,3R)-3-(2- (4-((1-(2,6-dioxopiperidin- 3-yl)-3-methyl-2-oxo-2,3- dihydro-1H-benzo[d]imid- azol-5-yl)methyl)piperidin- 1-yl)-2-oxoethoxy)-2-((R)- 3-phenylpiperidine-1- carbonyl)pyrrolidin-1-yl)- 3,3-dimethyl-1-oxobutan- 2-yl)carbamoyl)benzo[b] thiophen-5-yl)difluoro- methyl)phosphonic acid | 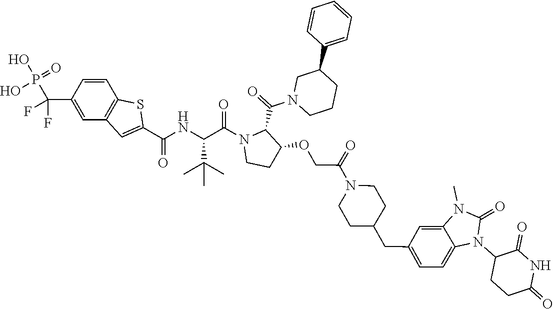 [SMILES: Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCC[C@H](c5ccccc5)C4)CC3)cc21] |
| 1.26 | ((1S)-(2-(((2S)-1-((2S,3R)- 3-(2-((1R,5S)-8-((1-(2,6- dioxopiperidin-3-yl)-3- methyl-2-oxo-2,3-dihydro- 1H-benzo[d]imidazol-5- yl)methyl)-3,8-diazabicyclo [3.2.1]octan-3-yl)-2-oxo- ethoxy)-2-((R)-2-phenyl- morpholine-4-carbonyl)- pyrrolidin-1-yl)-3,3- dimethyl-1-oxobutan-2-yl)- carbamoyl)-4-fluorobenzo [b]thiophen-5-yl)fluoro- methyl)phosphonic acid or ((1R)-(2-(((2S)-1-((2S,3R)- 3-(2-((1R,5S)-8-((1-(2,6- dioxopiperidin-3-yl)-3- methyl-2-oxo-2,3-dihydro- 1H-benzo[d]imidazol-5- yl)methyl)-3,8-diazabicyclo [3.2.1]octan-3-yl)-2-oxo- ethoxy)-2-((R)-2-phenyl- morpholine-4-carbonyl)- pyrrolidin-1-yl)-3,3-di- methyl-1-oxobutan-2-yl)- carbamoyl)-4-fluorobenzo [b]thiophen-5-yl)fluoro- methyl)phosphonic acid | 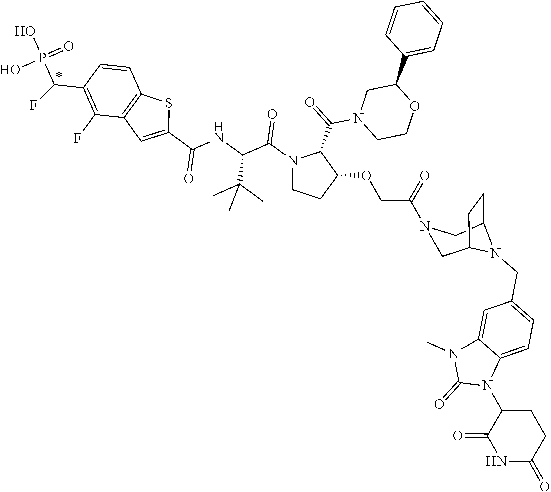 [SMILES: Cn1c(=O)n(C2CCC(=O)NC2=O)c2ccc(CN3C4CCC3CN(C(=O)CO[C@@H]3CCN(C(=O)[C@@H](NC(=O)c5cc6c(F)c(C(F)P(=O)(O)O)ccc6s5)C(C)(C)C)[C@@H]3C(=O)N3CCO[C@H](c5ccccc5)C3)C4)cc21] |
| 1.27 | ((2-(((2S)-1-((2S,3R)-3-(2- (4-((1-(2,6-dioxopiperidin- 3-yl)-3-methyl-2-oxo-2,3- dihydro-1H-benzo[d]imid- azol-4-yl)methyl)piperidin- 1-yl)-2-oxoethoxy)-2-((R)- 2-phenylmorpholine-4- carbonyl)pyrrolidin-1-yl)- 3,3-dimethyl-1-oxobutan- 2-yl)carbamoyl)benzo[b] thiophen-5-yl)difluoro- methyl)phosphonic acid | 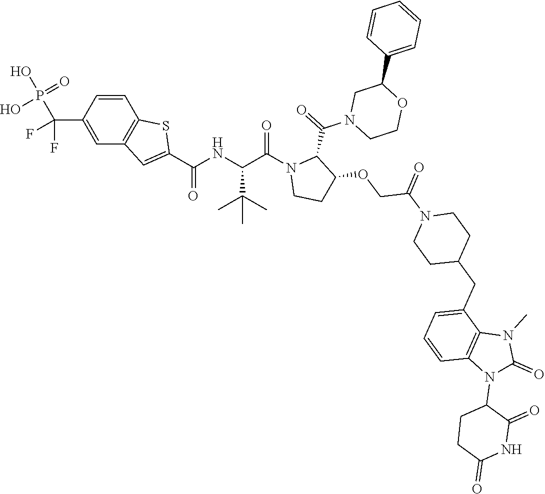 [SMILES: Cn1c(=O)n(C2CCC(=O)NC2=O)c2cccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)c21] |
| 1.28 | ((1S)-(2-(((2S)-1-((2S,3R)- 3-((1-((1-(2,6-dioxopiper- idin-3-yl)-3-methyl-2-oxo- 2,3-dihydro-1H-benzo[d] imidazol-5-yl)methyl)- piperidin-4-yl)methyl)-2- ((R)-2-phenylmorpholine- 4-carbonyl)pyrrolidin-1-yl)- 3,3-dimethyl-1-oxobutan- 2-yl)carbamoyl)-4-fluoro- benzo[b]thiophen-5-yl)- fluoromethyl)phosphonic acid or ((1R)-(2-(((2S)-1- ((2S,3R)-3-((1-((1-(2,6- dioxopiperidin-3-yl)-3- methyl-2-oxo-2,3-dihydro- 1H-benzo[d]imidazol-5- yl)methyl)piperidin-4-yl)- methyl)-2-((R)-2-phenyl- morpholine-4-carbonyl)- pyrrolidin-1-yl)-3,3-di- methyl-1-oxobutan-2-yl)- carbamoyl)-4-fluorobenzo [b]thiophen-5-yl)fluoro- methyl)phosphonic acid | 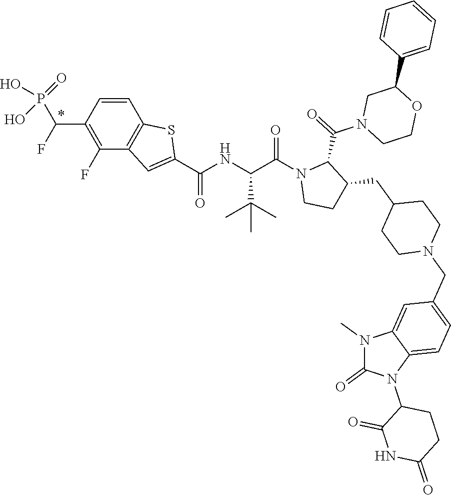 [SMILES: Cn1c(=O)n(C2CCC(=O)NC2=O)c2ccc(CN3CCC(C[C@@H]4CCN(C(=O)[C@@H](NC(=O)c5cc6c(F)c(C(F)P(=O)(O)O)ccc6s5)C(C)(C)C)[C@@H]4C(=O)N4CCO[C@H](c5ccccc5)C4)CC3)cc21] |
| 1.29 | ((1S)-(2-(((2S)-1-((2S,3R)- 3-(2-(2-((1-(2,6-dioxo- piperidin-3-yl)-3-methyl-2- oxo-2,3-dihydro-1H-benzo [d]imidazol-5-yl)methyl)- 2,7-diazaspiro[3.5]nonan-7- yl)-2-oxoethoxy)-2-((R)-2- phenylmorpholine-4- carbonyl)pyrrolidin-1-yl)- 3,3-dimethyl-1-oxobutan- 2-yl)carbamoyl)benzo[b] thiophen-5-yl)fluorometh- yl)phosphonic acid or ((1R)-(2-(((2S)-1-((2S,3R)- 3-(2-(2-((1-(2,6-dioxo- piperidin-3-yl)-3-methyl-2- oxo-2,3-dihydro-1H-benzo [d]imidazol-5-yl)methyl)- 2,7-diazaspiro[3.5]nonan-7- yl)-2-oxoethoxy)-2-((R)-2- phenylmorpholine-4- carbonyl)pyrrolidin-1-yl)- 3,3-dimethyl-1-oxobutan- 2-yl)carbamoyl)benzo[b] thiophen-5-l)fluoromethyl)- phosphonic acid | 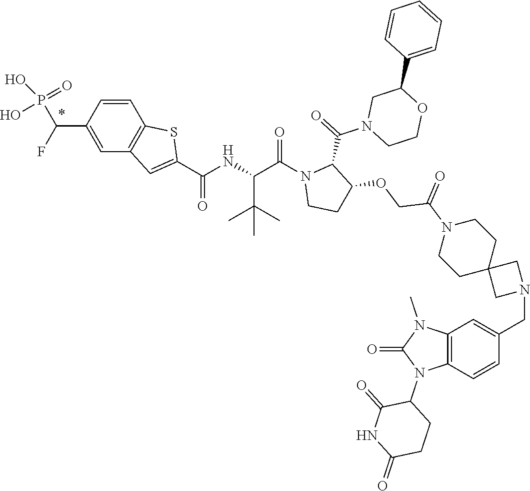 [SMILES: Cn1c(=O)n(C2CCC(=O)NC2=O)c2ccc(CN3CC4(CCN(C(=O)CO[C@@H]5CCN(C(=O)[C@@H](NC(=O)c6cc7cc(C(F)P(=O)(O)O)ccc7s6)C(C)(C)C)[C@@H]5C(=O)N5CCO[C@H](c6ccccc6)C5)CC4)C3)cc21] |
| 1.30 | ((1S)-(2-(((2S)-1-((2S,3R)- 3-(2-((1S,4S)-5-((1-(2,6- dioxopiperidin-3-yl)-3- methyl-2-oxo-2,3-dihydro- 1H-benzo[d]imidazol-5-yl)- methyl)-2,5-diazabicyclo [2.2.1]heptan-2-yl)-2-oxo- ethoxy)-2-((R)-2-phenyl- morpholine-4-carbonyl)pyr- rolidin-1-yl)-3,3-dimethyl- 1-oxobutan-2-yl)carbamo- yl)-benzo[b]thiophen-5-yl)- fluoromethyl)phosphonic acid or ((1R)-(2-(((2S)-1- ((2S,3R)-3-(2-((1S,4S)-5- ((1-(2,6-dioxopiperidin-3- yl)-3-methyl-2-oxo-2,3- dihydro-1H-benzo[d]imid- azol-5-yl)methyl)-2,5- diazabicyclo[2.2.1]heptan- 2-yl)-2-oxoethoxy)-2-((R)- 2-phenylmorpholine-4- carbonyl)pyrrolidin-1-yl)- 3,3-dimethyl-1-oxobutan-2- yl)carbamoyl)benzo[b]thio- phen-5-yl)fluoromethyl)- phosphonic acid | 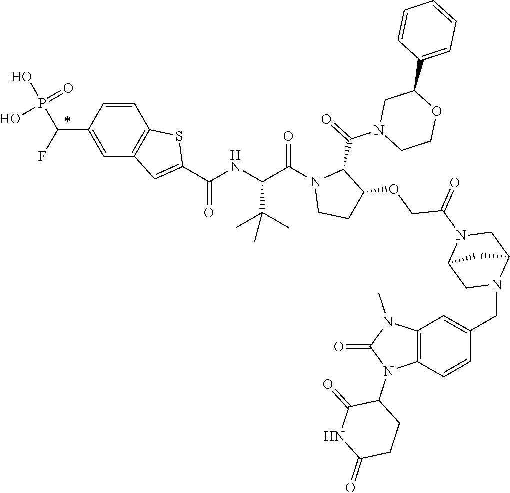 [SMILES: Cn1c(=O)n(C2CCC(=O)NC2=O)c2ccc(CN3C[C@@H]4C[C@H]3CN4C(=O)CO[C@@H]3CCN(C(=O)[C@@H](NC(=O)c4cc5cc(C(F)P(=O)(O)O)ccc5s4)C(C)(C)C)[C@@H]3C(=O)N3CCO[C@H](c4ccccc4)C3)cc21] |
| 1.31 | ((1S)-(2-(((2S)-1-((2S,3R)- 3-(2-(7-((1-(2,6-dioxopiper- idin-3-yl)-3-methyl-2-oxo- 2,3-dihydro-1H-benzo[d] imidazol-5-yl)methyl)-2,7- diazaspiro[3.5]nonan-2- yl)-2-oxoethoxy)-2-((R)- 2-phenylmorpholine-4- carbonyl)pyrrolidin-1-yl)- 3,3-dimethyl-1-oxobutan- 2-yl)carbamoyl)benzo[b] thiophen-5-yl)fluorometh- yl)phosphonic acid or ((1R)-(2-(((2S)-1-((2S,3R)- 3-(2-(7-((1-(2,6-dioxo- piperidin-3-yl)-3-methyl-2- oxo-2,3-dihydro-1H-benzo [d]imidazol-5-yl)methyl)- 2,7-diazaspiro[3.5]nonan- 2-yl)-2-oxoethoxy)-2-((R)- 2-phenylmorpholine-4- carbonyl)pyrrolidin-1-yl)- 3,3-dimethyl-1-oxobutan- 2-yl)carbamoyl)benzo[b] thiophen-5-yl)fluorometh- yl)phosphonic acid | 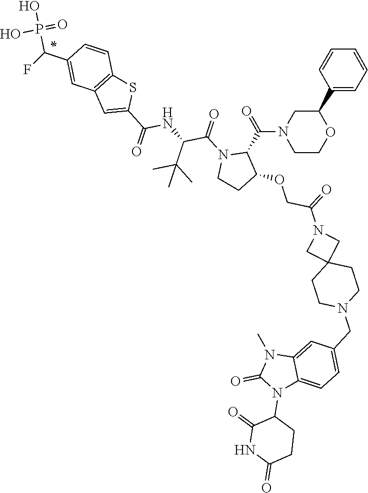 [SMILES: Cn1c(=O)n(C2CCC(=O)NC2=O)c2ccc(CN3CCC4(CC3)CN(C(=O)CO[C@@H]3CCN(C(=O)[C@@H](NC(=O)c5cc6cc(C(F)P(=O)(O)O)ccc6s5)C(C)(C)C)[C@@H]3C(=O)N3CCO[C@H](c5ccccc5)C3)C4)cc21] |
| 1.32 | ((1S)-(2-(((2S)-1-((2S,3R)- 3-(2-(6-((1-(2,6-dioxo- piperidin-3-yl)-3-methyl-2- oxo-2,3-dihydro-1H-benzo [d]imidazol-5-yl)methyl)- 3,6-diazabicyclo[3.1.1] heptan-3-yl)-2-oxoethoxy)- 2-((R)-2-phenylmorpholine- 4-carbonyl)pyrrolidin-1-yl)- 3,3-dimethyl-1-oxobutan-2- yl)carbamoyl)benzo[b]thio- phen-5-yl)fluoromethyl)- phosphonic acid or ((1R)- (2-(((2S)-1-((2S,3R)-3-(2- (6-((1-(2,6-dioxopiperidin- 3-yl)-3-methyl-2-oxo-2,3- dihydro-1H-benzo[d]imid- azol-5-yl)methyl)-3,6- diazabicyclo[3.1.1]heptan- 3-yl)-2-oxoethoxy)-2-((R)- 2-phenylmorpholine-4- carbonyl)pyrrolidin-1-yl)- 3,3-dimethyl-1-oxobutan- 2-yl)carbamoyl)benzo[b] thiophen-5-yl)fluorometh- yl)phosphonic acid | 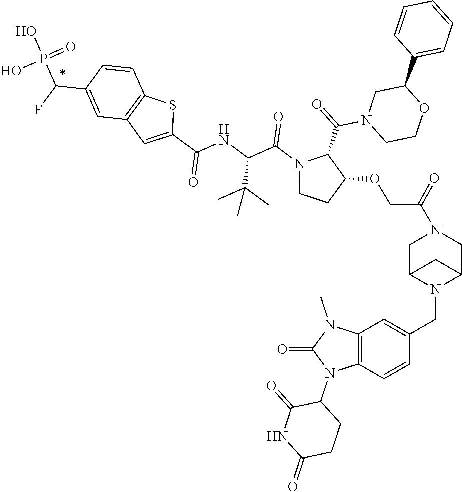 [SMILES: Cn1c(=O)n(C2CCC(=O)NC2=O)c2ccc(CN3C4CC3CN(C(=O)CO[C@@H]3CCN(C(=O)[C@@H](NC(=O)c5cc6cc(C(F)P(=O)(O)O)ccc6s5)C(C)(C)C)[C@@H]3C(=O)N3CCO[C@H](c5ccccc5)C3)C4)cc21] |
| 1.33 | ((1R)-(2-(((2S)-1-((2S,3R)- 3-(2-((1R,5S)-8-((1-(2,6- dioxopiperidin-3-yl)-3- methyl-2-oxo-2,3-dihydro- 1H-benzo[d]imidazol-5- yl)methyl)-3,8-diazabicyclo [3.2.1]octan-3-yl)-2-oxoeth- oxy)-2-((R)-2-phenylmor- pholine-4-carbonyl)pyrroli- din-1-yl)-3,3-dimethyl-1- oxobutan-2-yl)carbamoyl)- benzo[b]thiophen-5-yl)- fluoromethyl)phosphonic acid or ((1S)-(2-(((2S)-1- ((2S,3R)-3-(2-((1R,5S)-8- ((1-(2,6-dioxopiperidin-3- yl)-3-methyl-2-oxo-2,3- dihydro-1H-benzo[d]imid- azol-5-yl)methyl)-3,8- diazabicyclo[3.2.1]octan- 3-yl)-2-oxoethoxy)-2-((R)- 2-phenylmorpholine-4- carbonyl)pyrrolidin-1-yl)- 3,3-dimethyl-1-oxobutan- 2-yl)carbamoyl)benzo[b] thiophen-5-yl)fluorometh- yl)phosphonic acid | 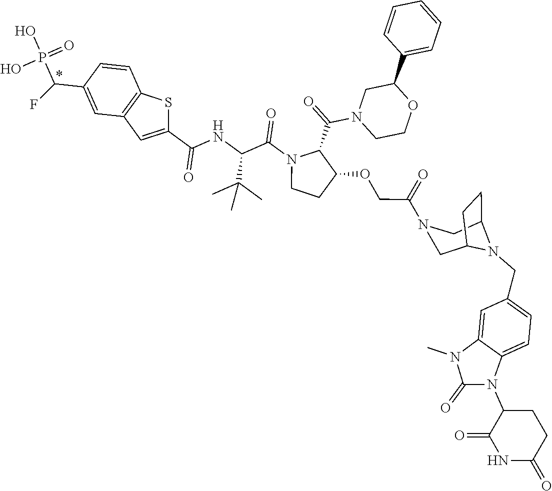 [SMILES: Cn1c(=O)n(C2CCC(=O)NC2=O)c2ccc(CN3C4CCC3CN(C(=O)CO[C@@H]3CCN(C(=O)[C@@H](NC(=O)c5cc6cc(C(F)P(=O)(O)O)ccc6s5)C(C)(C)C)[C@@H]3C(=O)N3CCO[C@H](c5ccccc5)C3)C4)cc21] |
| 1.34 | ((1S)-(2-(((2S)-1-((2S,3R)- 3-(2-(4-((1-(2,6-dioxo- piperidin-3-yl)-3-methyl-2- oxo-2,3-dihydro-1H-benzo [d]imidazol-5-yl)methyl)- piperazin-1-yl)-2-oxoeth- oxy)-2-((R)-2-phenylmor- pholine-4-carbonyl)pyrroli- din-1-yl)-3,3-dimethyl-1- oxobutan-2-yl)carbamoyl)- benzo[b]thiophen-5-yl)- fluoromethyl)phosphonic acid or ((1R)-(2-(((2S)-1- ((2S,3R)-3-(2-(4-((1-(2,6- dioxopiperidin-3-yl)-3- methyl-2-oxo-2,3-dihydro- 1H-benzo[d]imidazol-5- yl)methyl)piperazin-1-yl)- 2-oxoethoxy)-2-((R)-2- phenylmorpholine-4-car- bonyl)pyrrolidin-1-yl)-3,3- dimethyl-1-oxobutan-2-yl)- carbamoyl)benzo[b]thio- phen-5-yl)fluoromethyl)- phosphonic acid | 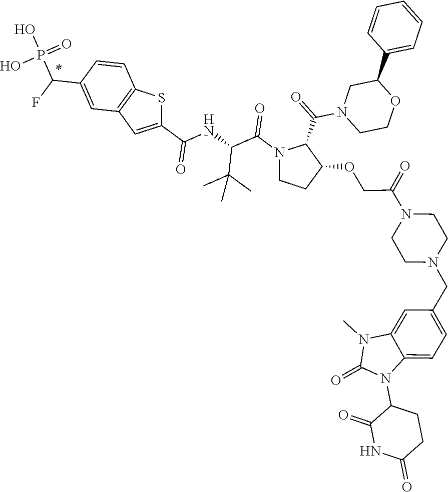 [SMILES: Cn1c(=O)n(C2CCC(=O)NC2=O)c2ccc(CN3CCN(C(=O)CO[C@@H]4CCN(C(=O)[C@@H](NC(=O)c5cc6cc(C(F)P(=O)(O)O)ccc6s5)C(C)(C)C)[C@@H]4C(=O)N4CCO[C@H](c5ccccc5)C4)CC3)cc21] |
| 1.35 | ((1R)-(2-(((2S)-1-((2S,3R)- 3-(2-(2-((1-(2,6-dioxo- piperidin-3-yl)-3-methyl-2- oxo-2,3-dihydro-1H-benzo [d]imidazol-5-yl)methyl)- 2,6-diazaspiro[3.4]octan-6- yl)-2-oxoethoxy)-2-((R)-2- phenylmorpholine-4-car- bonyl)pyrrolidin-1-yl)-3,3- dimethyl-1-oxobutan-2-yl)- carbamoyl)benzo[b]thio- phen-5-yl)fluoromethyl)- phosphonic acid or ((1S)- (2-(((2S)-1-((2S,3R)-3-(2- (2-((1-(2,6-dioxopiperidin- 3-yl)-3-methyl-2-oxo-2,3- dihydro-1H-benzo[d]imid- azol-5-yl)methyl)-2,6- diazaspiro[3.4]octan-6-yl)- 2-oxoethoxy)-2-((R)-2- phenylmorpholine-4- carbonyl)pyrrolidin-1-yl)- 3,3-dimethyl-1-oxobutan- 2-yl)carbamoyl)benzo[b] thiophen-5-l)fluoromethyl)- phosphonic acid | 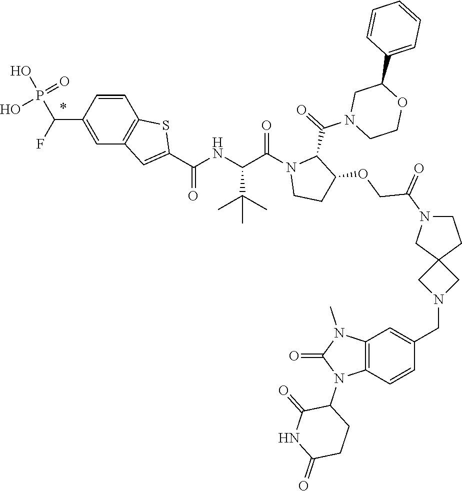 [SMILES: Cn1c(=O)n(C2CCC(=O)NC2=O)c2ccc(CN3CC4(CCN(C(=O)CO[C@@H]5CCN(C(=O)[C@@H](NC(=O)c6cc7cc(C(F)P(=O)(O)O)ccc7s6)C(C)(C)C)[C@@H]5C(=O)N5CCO[C@H](c6ccccc6)C5)C4)C3)cc21] |
| 1.36 | ((1S)-(2-(((2S)-1-((2S,3R)- 3-(2-((1R,4R)-5-((1-(2,6- dioxopiperidin-3-yl)-3- methyl-2-oxo-2,3-dihydro- 1H-benzo[d]imidazol-5- yl)methyl)-2,5-diazabicyclo [2.2.1]heptan-2-yl)-2-oxo- ethoxy)-2-((R)-2-phenyl- morpholine-4-carbonyl)- pyrrolidin-1-yl)-3,3-di- methyl-1-oxobutan-2-yl)- carbamoyl)benzo[b]thio- phen-5-yl)fluoromethyl)- phosphonic acid or ((1R)- (2-(((2S)-1-((2S,3R)-3-(2- ((1R,4R)-5-((1-(2,6-dioxo- piperidin-3-yl)-3-methyl-2- oxo-2,3-dihydro-1H-benzo [d]imidazol-5-yl)methyl)- 2,5-diazabicyclo[2.2.1] heptan-2-yl)-2-oxoethoxy)- 2-((R)-2-phenylmorpho- line-4-carbonyl)pyrrolidin- 1-yl)-3,3-dimethyl-1-oxo- butan-2-yl)carbamoyl)- benzo[b]thiophen-5-yl)- fluoromethyl)phosphonic acid | 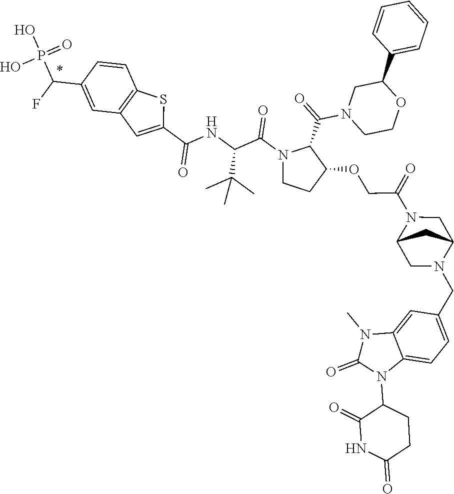 [SMILES: Cn1c(=O)n(C2CCC(=O)NC2=O)c2ccc(CN3C[C@H]4C[C@@H]3CN4C(=O)CO[C@@H]3CCN(C(=O)[C@@H](NC(=O)c4cc5cc(C(F)P(=O)(O)O)ccc5s4)C(C)(C)C)[C@@H]3C(=O)N3CCO[C@H](c4ccccc4)C3)cc21] |
| 1.37 | ((1S)-(2-(((2S)-1-((2S,3R)- 3-(2-((1R,5R)-3-((1-(2,6- dioxopiperidin-3-yl)-3- methyl-2-oxo-2,3-dihydro- 1H-benzo[d]imidazol-5- yl)methyl)-3,8-diazabi- cyclo[3.2.1]octan-8-yl)-2- oxoethoxy)-2-((R)-2-phen- ylmorpholine-4-carbonyl)- pyrrolidin-1-yl)-3,3-di- methyl-1-oxobutan-2-yl)- carbamoyl)benzo[b]thio- phen-5-yl)fluoromethyl)- phosphonic acid or ((1R)- (2-(((2S)-1-((2S,3R)-3-(2- ((1R,5R)-3-((1-(2,6-dioxo- piperidin-3-yl)-3-methyl-2- oxo-2,3-dihydro-1H-benzo [d]imidazol-5-yl)methyl)- 3,8-diazabicyclo[3.2.1] octan-8-yl)-2-oxoethoxy)-2- ((R)-2-phenylmorpholine- 4-carbonyl)pyrrolidin-1-yl)- 3,3-dimethyl-1-oxobutan-2- yl)carbamoyl)benzo[b]thio- phen-5-yl)fluoromethyl)- phosphonic acid | 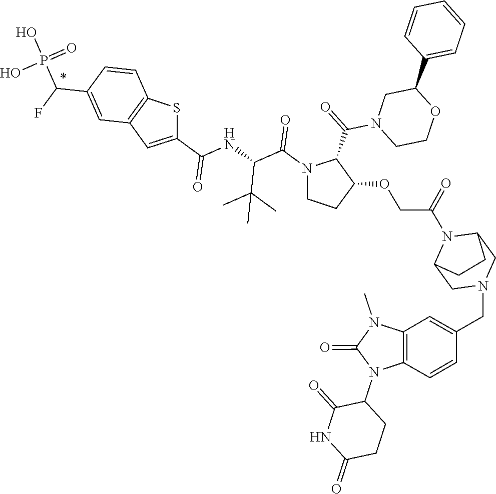 [SMILES: Cn1c(=O)n(C2CCC(=O)NC2=O)c2ccc(CN3CC4CCC(C3)N4C(=O)CO[C@@H]3CCN(C(=O)[C@@H](NC(=O)c4cc5cc(C(F)P(=O)(O)O)ccc5s4)C(C)(C)C)[C@@H]3C(=O)N3CCO[C@H](c4ccccc4)C3)cc21] |
| 1.38 | ((1R)-(2-(((2S)-1-((2S,3R)- 3-(2-(3-((1-(2,6-dioxo- piperidin-3-yl)-3-methyl-2- oxo-2,3-dihydro-1H-benzo [d]imidazol-5-yl)methyl)- 3,6-diazabicyclo[3.1.1] heptan-6-yl)-2-oxoethoxy)- 2-((R)-2-phenylmorpholine- 4-carbonyl)pyrrolidin-1-yl)- 3,3-dimethyl-1-oxobutan-2- yl)carbamoyl)benzo[b]thio- phen-5-yl)fluoromethyl) phosphonic acid or ((1S)- (2-(((2S)-1-((2S,3R)-3-(2- (3-((1-(2,6-dioxopiperidin- 3-yl)-3-methyl-2-oxo-2,3- dihydro-1H-benzo[d]imid- azol-5-yl)methyl)-3,6- diazabicyclo[3.1.1]heptan- 6-yl)-2-oxoethoxy)-2-((R)- 2-phenylmorpholine-4- carbonyl)pyrrolidin-1-yl)- 3,3-dimethyl-1-oxobutan-2- yl)carbamoyl)benzo[b]thio- phen-5-yl)fluoromethyl)- phosphonic acid | 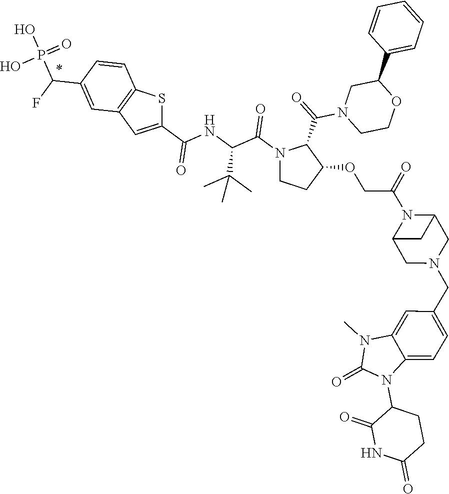 [SMILES: Cn1c(=O)n(C2CCC(=O)NC2=O)c2ccc(CN3CC4CC(C3)N4C(=O)CO[C@@H]3CCN(C(=O)[C@@H](NC(=O)c4cc5cc(C(F)P(=O)(O)O)ccc5s4)C(C)(C)C)[C@@H]3C(=O)N3CCO[C@H](c4ccccc4)C3)cc21] |
| 1.39 | ((2-(((2S)-1-((2S,3R)-3-(2- (4-(4-(1-(2,6-dioxopiperi- din-3-yl)-3-methyl-2-oxo- 2,3-dihydro-1H-benzo[d] imidazol-5-yl)piperazin-1- yl)piperidin-1-yl)-2-oxo- ethoxy)-2-((R)-2-phenyl- morpholine-4-carbonyl)- pyrrolidin-1-yl)-3,3-di- methyl-1-oxobutan-2-yl)- carbamoyl)benzo[b]thio- phen-5-yl)difluoromethyl)- phosphonic acid | 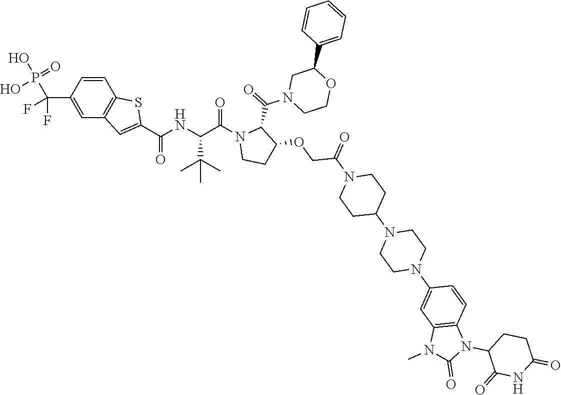 [SMILES: Cn1c(=O)n(C2CCC(=O)NC2=O)c2ccc(N3CCN(C4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21] |
| 1.40 | ((1R)-(2-(((2S)-1-((2S,3R)- 3-(2-((5R)-7-((1-(2,6- dioxopiperidin-3-yl)-3- methyl-2-oxo-2,3-dihydro- 1H-benzo[d]imidazol-5- yl)methyl)-2,7-diazaspiro [4.4]nonan-2-yl)-2-oxo- ethoxy)-2-((R)-2-phenyl- morpholine-4-carbonyl)- pyrrolidin-1-yl)-3,3-di- methyl-1-oxobutan-2-yl)- carbamoyl)benzo[b]thio- phen-5-yl)fluoromethyl)- phosphonic acid or ((1S)- (2-(((2S)-1-((2S,3R)-3-(2- ((5R)-7-((1-(2,6-dioxo- piperidin-3-yl)-3-methyl-2- oxo-2,3-dihydro-1H-benzo [d]imidazol-5-yl)methyl)- 2,7-diazaspiro[4.4]nonan- 2-yl)-2-oxoethoxy)-2-((R)- 2-phenylmorpholine-4- carbonyl)pyrrolidin-1-yl)- 3,3-dimethyl-1-oxobutan- 2-yl)carbamoyl)benzo[b] thiophen-5-yl)fluorometh- yl)phosphonic acid | 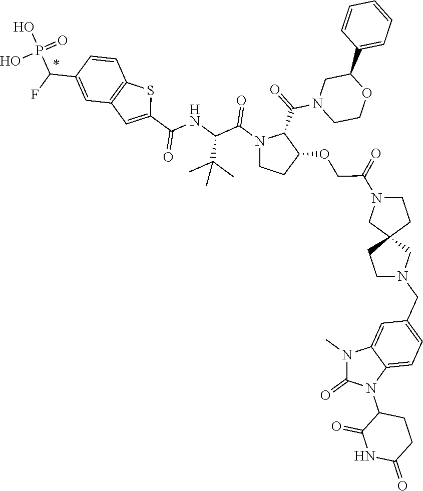 [SMILES: Cn1c(=O)n(C2CCC(=O)NC2=O)c2ccc(CN3CC[C@@]4(CCN(C(=O)CO[C@@H]5CCN(C(=O)[C@@H](NC(=O)c6cc7cc(C(F)P(=O)(O)O)ccc7s6)C(C)(C)C)[C@@H]5C(=O)N5CCO[C@H](c6ccccc6)C5)C4)C3)cc21] |
| 1.41 | ((1S)-(2-(((2S)-1-((2S,3R)- 3-(2-((3aR,6aS)-5-((1-(2,6- dioxopiperidin-3-yl)-3- methyl-2-oxo-2,3-dihydro- 1H-benzo[d]imidazol-5- yl)methyl)hexahydropyr- rolo[3,4-c]pyrrol-2(1H)-yl)- 2-oxoethoxy)-2-((R)-2- phenylmorpholine-4- carbonyl)pyrrolidin-1-yl)- 3,3-dimethyl-1-oxobutan- 2-yl)carbamoyl)benzo[b] thiophen-5-yl)fluorometh- yl)phosphonic acid or ((1R)-(2-(((2S)-1-((2S,3R)- 3-(2-((3aR,6aS)-5-((1-(2,6- dioxopiperidin-3-yl)-3- methyl-2-oxo-2,3-dihydro- 1H-benzo[d]imidazol-5- yl)methyl)hexahydropyr- rolo[3,4-c]pyrrol-2(1H)-yl)- 2-oxoethoxy)-2-((R)-2- phenylmorpholine-4- carbonyl)pyrrolidin-1-yl)- 3,3-dimethyl-1-oxobutan- 2-yl)carbamoyl)benzo[b] thiophen-5-yl)fluorometh- yl)phosphonic acid | 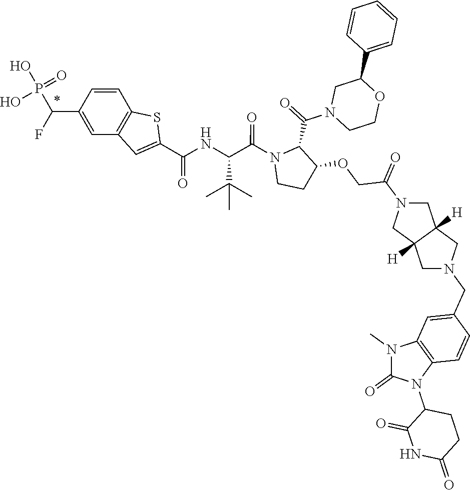 [SMILES: Cn1c(=O)n(C2CCC(=O)NC2=O)c2ccc(CN3C[C@@H]4CN(C(=O)CO[C@@H]5CCN(C(=O)[C@@H](NC(=O)c6cc7cc(C(F)P(=O)(O)O)ccc7s6)C(C)(C)C)[C@@H]5C(=O)N5CCO[C@H](c6ccccc6)C5)C[C@@H]4C3)cc21] |
| 1.42 | ((1S)-(2-(((2S)-1-((2S,3R)- 3-(2-((5S)-7-((1-(2,6-dioxo- piperidin-3-yl)-3-methyl-2- oxo-2,3-dihydro-1H-benzo [d]imidazol-5-yl)methyl)- 2,7-diazaspiro[4.4]nonan-2- yl)-2-oxoethoxy)-2-((R)- 2-phenylmorpholine-4- carbonyl)pyrrolidin-1-yl)- 3,3-dimethyl-1-oxobutan- 2-yl)carbamoyl)benzo[b] thiophen-5-yl)fluorometh- yl)phosphonic acid or ((1R)-(2-(((2S)-1-((2S,3R)- 3-(2-((5S)-7-((1-(2,6-dioxo- piperidin-3-yl)-3-methyl-2- oxo-2,3-dihydro-1H-benzo [d]imidazol-5-yl)methyl)- 2,7-diazaspiro[4.4]nonan- 2-yl)-2-oxoethoxy)-2-((R)- 2-phenylmorpholine-4- carbonyl)pyrrolidin-1-yl)- 3,3-dimethyl-1-oxobutan- 2-yl)carbamoyl)benzo[b] thiophen-5-yl)fluorometh- yl)phosphonic acid | 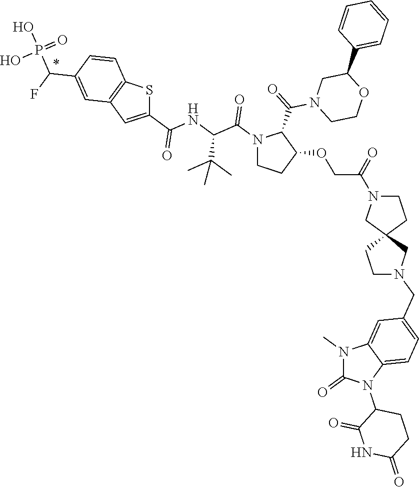 [SMILES: Cn1c(=O)n(C2CCC(=O)NC2=O)c2ccc(CN3CC[C@]4(CCN(C(=O)CO[C@@H]5CCN(C(=O)[C@@H](NC(=O)c6cc7cc(C(F)P(=O)(O)O)ccc7s6)C(C)(C)C)[C@@H]5C(=O)N5CCO[C@H](c6ccccc6)C5)C4)C3)cc21] |
| 1.43 | ((1S)-(2-(((2S)-1-((2S,3R)- 3-(2-(6-((1-(2,6-dioxo- piperidin-3-yl)-3-methyl-2- oxo-2,3-dihydro-1H-benzo [d]imidazol-5-yl)methyl)- 2,6-diazaspiro[3.4]octan-2- yl)-2-oxoethoxy)-2-((R)-2- phenylmorpholine-4- carbonyl)pyrrolidin-1-yl)- 3,3-dimethyl-1-oxobutan- 2-yl)carbamoyl)benzo[b] thiophen-5-yl)fluorometh- yl)phosphonic acid or ((1R)-(2-(((2S)-1-((2S,3R)- 3-(2-(6-((1-(2,6-dioxo- piperidin-3-yl)-3-methyl-2- oxo-2,3-dihydro-1H-benzo [d]imidazol-5-yl)methyl)- 2,6-diazaspiro[3.4]octan-2- yl)-2-oxoethoxy)-2-((R)-2- phenylmorpholine-4- carbonyl)pyrrolidin-1-yl)- 3,3-dimethyl-1-oxobutan- 2-yl)carbamoyl)benzo[b] thiophen-5-yl)fluorometh- yl)phosphonic acid | 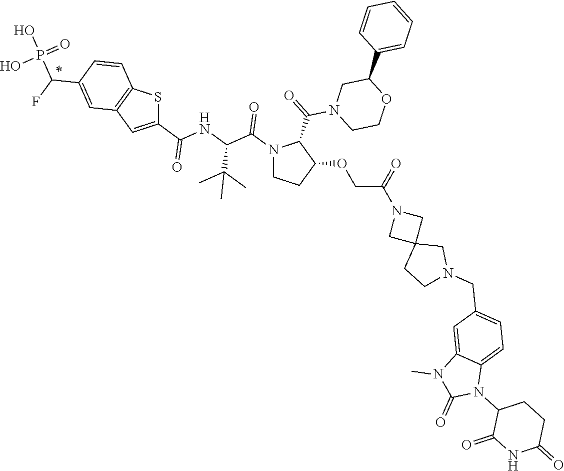 [SMILES: Cn1c(=O)n(C2CCC(=O)NC2=O)c2ccc(CN3CCC4(C3)CN(C(=O)CO[C@@H]3CCN(C(=O)[C@@H](NC(=O)c5cc6cc(C(F)P(=O)(O)O)ccc6s5)C(C)(C)C)[C@@H]3C(=O)N3CCO[C@H](c5ccccc5)C3)C4)cc21] |
| 1.44 | ((2-(((2S)-1-((2S,3R)-3-((2- (4-((1-(2,6-dioxopiperidin- 3-yl)-3-methyl-2-oxo-2,3- dihydro-1H-benzo[d]imid- azol-5-yl)methyl)piperidin- 1-yl)-2-oxoethyl)amino)-2- ((R)-2-phenylmorpholine- 4-carbonyl)pyrrolidin-1-yl)- 3,3-dimethyl-1-oxobutan- 2-yl)carbamoyl)benzo[b] thiophen-5-yl)difluorometh- yl)phosphonic acid | 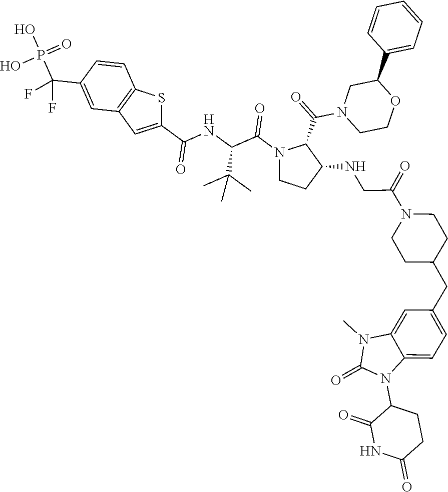 [SMILES: Cn1c(=O)n(C2CCC(=O)NC2=O)c2ccc(CC3CCN(C(=O)CN[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21] |
| 1.45 | ((1R)-(2-(((2S)-1-((2S,3R)- 3-(2-(4-((1-(2,6-dioxo- piperidin-3-yl)-3-methyl-2- oxo-2,3-dihydro-1H-benzo [d]imidazol-5-yl)methyl)- piperidin-1-yl)-2-oxoeth- oxy)-2-((R)-2-phenylmor- pholine-4-carbonyl)pyrroli- din-1-yl)-3,3-dimethyl-1- oxobutan-2-yl)carbamoyl)- 4-fluorobenzo[b]thiophen- 5-yl)fluoromethyl)phos- phonic acid or ((1S)-(2- (((2S)-1-((2S,3R)-3-(2-(4- ((1-(2,6-dioxopiperidin-3- yl)-3-methyl-2-oxo-2,3- dihydro-1H-benzo[d]imid- azol-5-yl)methyl)piperidin- 1-yl)-2-oxoethoxy)-2-((R)- 2-phenylmorpholine-4- carbonyl)pyrrolidin-1-yl)- 3,3-dimethyl-1-oxobutan- 2-yl)carbamoyl)-4-fluoro- benzo[b]thiophen-5-yl)- fluoromethyl)phosphonic acid | 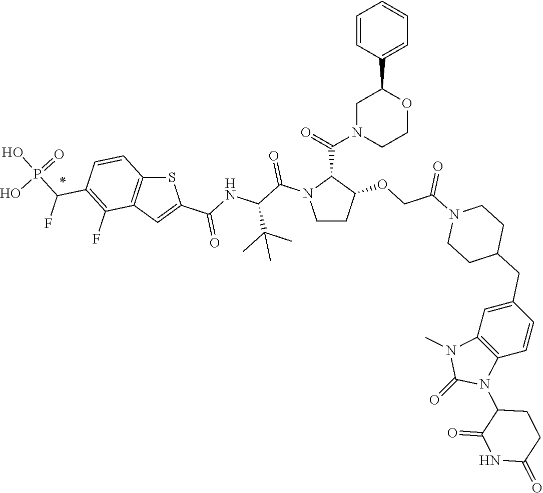 [SMILES: Cn1c(=O)n(C2CCC(=O)NC2=O)c2ccc(CC3CCN(C(=O)CO[C@@H]4CCN(C(=O)[C@@H](NC(=O)c5cc6c(F)c(C(F)P(=O)(O)O)ccc6s5)C(C)(C)C)[C@@H]4C(=O)N4CCO[C@H](c5ccccc5)C4)CC3)cc21] |
| 1.46 | ((2-(((2S)-1-((2S,3R)-3-((1- ((1-(2,6-dioxopiperidin-3- yl)-3-methyl-2-oxo-2,3- dihydro-1H-benzo[d]imid- azol-5-yl)methyl)piperidin- 4-yl)methyl)-2-((R)-2- phenylmorpholine-4- carbonyl)pyrrolidin-1-yl)- 3,3-dimethyl-1-oxobutan- 2-yl)carbamoyl)-4-fluoro- benzo[b]thiophen-5-yl)- difluoromethyl)phosphonic acid | 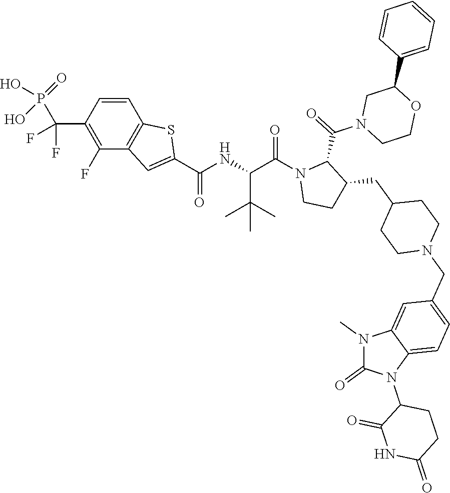 [SMILES: Cn1c(=O)n(C2CCC(=O)NC2=O)c2ccc(CN3CCC(C[C@@H]4CCN(C(=O)[C@@H](NC(=O)c5cc6c(F)c(C(F)(F)P(=O)(O)O)ccc6s5)C(C)(C)C)[C@@H]4C(=O)N4CCO[C@H](c5ccccc5)C4)CC3)cc21] |

TABLE 1.2
Procedures and analogs to Example 1.1 Synthesis.

| Example | Starting | Instead of | Variation from |
|---|---|---|---|
| Number | Material | A3.2 | Example 1.1 |
| 1.2 | C1.5 | A1.2 | |
| 1.3 | C1.5 | A2.1 | Step 1 run for 16 h |
| 1.4 | C1.5 | A3.4 | |
| 1.5 | C1.5 | A3.5 | Step 1 run for 16 h |
| 1.6 | C1.5 | A4.2 | Step 1 run for 16 h |
| 1.7 | C1.5 | A4.1 | Step 1 run for 16 h |
| 1.8 | C1.5 | A4.3 | 15 eq. of DIPEA used in step 1 |
| 1.9 | C4.7 | A4.4 | |
| 1.10 | C1.5 | A4.7 | DMF used instead of MeCN |
| | | | in step 1 |
| 1.11 | C1.5 | A4.6 | DMF used instead of MeCN |
| | | | in step 1 |
| 1.12 | C1.5 | A4.5 | |
| 1.13 | C1.5 | A4.8 | |
| 1.14 | C1.5 | A2.2 | Step 1 run for 16 h |
| 1.15 | C1.5 | A4.9 | |
| 1.16 | C1.5 | A4.10 | |
| 1.17 | C3.6 | D1.4 | |
| 1.18 | C3.6 | D1.3 | |
| 1.19 | C3.6 | D4.3 | |
| 1.20 | C1.5 | A7.3 | |
| 1.21 | C3.6 | D5.1 | |
| 1.22 | C3.6 | D3.1 | |
| 1.23 | C1.5 | A8.2 | |
| 1.24 | C9.1 | B8.3 | |
| 1.25 | C3.6 | (R)-3- | |
| | | phenylpiperidine | |
| 1.26 | C11.1 | B8.3 | |
| 1.27 | C1.5 | A3.6 | |
| 1.28 | C5.3 | B8.3 | Head coupling |
| 1.29 | C10.1 | A9.1 | |
| 1.30 | C10.1 | A10.1 | |
| 1.31 | C10.1 | A11.1 | |
| 1.32 | C10.1 | A12.1 | |
| 1.33 | C10.1 | A1.2 | |
| 1.34 | C10.1 | A4.10 | |
| 1.35 | C10.1 | A13.1 | |
| 1.36 | C10.1 | A14.1 | |
| 1.37 | C10.1 | A15.1 | |
| 1.38 | C10.1 | A16.1 | |
| 1.39 | C1.5 | A17.1 | |
| 1.40 | C10.1 | A20.1 | |
| 1.41 | C10.1 | A19.1 | |
| 1.42 | C10.1 | A18.1 | |
| 1.43 | C10.1 | A21.1 | |
| 1.44 | E1.2 | A3.4 | |
| 1.45 | C2.2 | B8.3 | |
| 1.46 | C5.3 | B8.5 | |

TABLE 1.3
ES/MS and 1H NMR data for Examples 1.1 to 1.46

| Example | ES/MS | | |
|---|---|---|---|
| Number | m/z | Ion | 1H NMR |
| 1.1 | 1059.7 | [M − H]⁻ | (400 MHz, DMSO-d6) δ 11.08 (s, 1H), 8.58- |
| | | | 8.43 (m, 1H), 8.40-8.23 (m, 1H), 8.18- |
| | | | 8.09 (m, 1H), 8.06 (s, 1H), 7.64-7.57 (m, |
| | | | 1H), 7.47-6.87 (m, 8H), 5.38-5.07 (m, 2H), |
| | | | 4.80-0.87 (m, 40H). |
| 1.2 | 1101.2 | [M − H]⁻ | (500 MHz, DMSO-d6, 80° C.) δ 10.87 (s, 1H), |
| | | | 8.43 (s, 1H), 8.18-7.92 (m, 3H), 7.68-7.60 |
| | | | (m, 1H), 7.48-7.28 (m, 6H), 7.28-7.16 (m, |
| | | | 2H), 5.37 (dd, J = 12.7, 5.4 Hz, 1H), 4.77 (d, |
| | | | J = 8.8 Hz, 1H), 4.48-3.45 (m, 20H), 2.97- |
| | | | 2.87 (m, 3H), 2.80-2.63 (m, 4H), 2.33-2.02 |
| | | | (m, 7H), 1.18-1.01 (m, 9H). |
| 1.3 | 1116.3 | [M − H]⁻ | (400 MHz, DMSO-d6) δ 11.07 (s, 1H), 8.52 |
| | | | (d, J = 10.6 Hz, 1H), 8.36-8.20 (m, 1H), 8.14 |
| | | | (d, J = 8.5 Hz, 1H), 8.05 (s, 1H), 7.63-7.54 |
| | | | (m, 1H), 7.44-7.23 (m, 5H), 7.02-6.92 (m, |
| | | | 2H), 6.85-6.74 (m, 1H), 5.37-5.15 (m, 2H), |
| | | | 5.13-2.36 (m, 23H), 2.24-1.89 (m, 3H), |
| | | | 1.71-1.29 (m, 7H), 1.17-0.64 (m, 15H). |
| 1.4 | 1073.8 | [M − H]⁻ | (400 MHz, DMSO-d6) δ 11.12 (s, 1H), 8.65- |
| | | | 8.47 (m, 1H), 8.43-8.26 (m, 1H), 8.23- |
| | | | 8.14 (m, 1H), 8.09 (s, 1H), 7.66-7.58 (m, |
| | | | 1H), 7.52-7.23 (m, 5H), 7.09-6.97 (m, 2H), |
| | | | 6.93-6.80 (m, 1H), 5.42-5.07 (m, 2H), 4.88- |
| | | | 1.49 (m, 33H), 1.30-0.89 (m, 9H). |
| 1.5 | 1142.8 | [M − H]⁻ | (400 MHz, DMSO-d6) δ 11.09 (s, 1H), 9.26 |
| | | | (br. s, 1H), 8.57-8.43 (m, 1H), 8.40-8.24 |
| | | | (m, 1H), 8.13 (d, J = 8.6 Hz, 1H), 8.05 (s, 1H), |
| | | | 7.59 (d, J = 8.7 Hz, 1H), 7.50-7.22 (m, 5H), |
| | | | 7.12-6.96 (m, 2H), 6.91 (d, J = 7.8 Hz, 1H), |
| | | | 5.41-4.99 (m, 2H), 4.83-4.65 (m, 1H), 4.62- |
| | | | 1.30 (m, 39H), 1.16-0.96 (m, 9H). |
| 1.6 | 1161.5 | [M + H]⁺ | (400 MHz, DMSO-d6) δ 11.10 (s, 1H), 8.52 |
| | | | (d, J = 12.4 Hz, 1H), 8.40-8.24 (m, 1H), 8.17- |
| | | | 7.99 (m, 2H), 7.63-7.55 (m, 1H), 7.46- |
| | | | 6.81 (m, 8H), 5.51-5.01 (m, 3H), 4.93-1.57 |
| | | | (m, 36H), 1.56-1.30 (m, 2H), 1.15-0.98 (m, |
| | | | 10H). |
| 1.7 | 1142.7 | [M − H]⁻ | |
| 1.8 | 1159.5 | [M + H]⁺ | (400 MHz, DMSO-d6) δ 11.10 (s, 1H), 8.52 |
| | | | (d, J = 12.2 Hz, 1H), 8.40-8.18 (m, 1H), 8.13 |
| | | | (d, J = 8.5 Hz, 1H), 8.05 (s, 1H), 7.59 (d, J = |
| | | | 8.8 Hz, 1H), 7.47-6.84 (m, 8H), 5.40-5.02 |
| | | | (m, 2H), 4.81-4.67 (m, 1H), 4.57-0.76 (m, |
| | | | 50H). |
| 1.9 | 1043.8 | [M − H]⁻ | (400 MHz, DMSO-d6) δ 11.15 (s, 1H), 8.66- |
| | | | 8.02 (m, 5H), 7.73-7.55 (m, 1H), 7.55- |
| | | | 7.01 (m, 6H), 5.49-5.10 (m, 2H), 5.09-0.85 |
| | | | (m, 40H). |
| 1.10 | 1060.7 | [M − H]⁻ | (400 MHz, DMSO-d6) δ 11.07 (s, 1H), 8.53 |
| | | | (d, J = 11.8 Hz, 1H), 8.41-8.21 (m, 1H), 8.18- |
| | | | 8.01 (m, 2H), 7.59 (d, J = 8.6 Hz, 1H), 7.45- |
| | | | 7.40 (m, 1H), 7.38-7.24 (m, 4H), 7.03- |
| | | | 6.86 (m, 2H), 6.74-6.61 (m, 1H), 5.36-5.19 |
| | | | (m, 2H), 5.16-4.67 (m, 2H), 4.62-2.42 (m, |
| | | | 25H), 2.30-1.91 (m, 3H), 1.06 (dd, J = 25.2, |
| | | | 14.8 Hz, 9H). |
| 1.11 | 1143.8 | [M − H]⁻ | (400 MHz, DMSO-d6) δ 11.08 (s, 1H), 9.55 |
| | | | (s, 1H), 8.59-8.46 (m, 1H), 8.42-8.24 (m, |
| | | | 1H), 8.18-8.02 (m, 2H), 7.60 (d, J = 8.7 Hz, |
| | | | 1H), 7.51-7.15 (m, 5H), 7.04-6.87 (m, 2H), |
| | | | 6.71 (d, J = 8.6 Hz, 1H), 5.35-5.16 (m, 2H), |
| | | | 4.77 (d, J = 10.3 Hz, 1H), 4.61-2.40 (m, |
| | | | 31H), 2.27-1.87 (m, 5H), 1.55 (m, 2H), 1.07 |
| | | | (m, 9H). |
| 1.12 | 1089.2 | [M − H]⁻ | (500 MHz, DMSO-d6, 80° C.) δ 10.87 (br. s, |
| | | | 1H), 8.43 (s, 1H), 8.14-7.95 (m, 3H), 7.64 |
| | | | (d, J = 8.7 Hz, 1H), 7.46-7.09 (m, 7H), 5.42- |
| | | | 5.29 (m, 1H), 4.80-4.72 (m, 1H), 4.54- |
| | | | 3.43 (m, 23H), 2.96-2.63 (m, 4H), 2.27- |
| | | | 2.03 (m, 4H), 1.16-1.00 (m, 9H). |
| 1.13 | 1087.2 | [M − H]⁻ | (400 MHz, DMSO-d6) δ 11.06 (s, 1H), 8.68- |
| | | | 6.52 (m, 10H), 5.41-1.47 (m, 37H), 1.15- |
| | | | 0.99 (m, 9H). |
| 1.14 | 1102.2 | [M − H]⁻ | (400 MHz, DMSO-d6) δ 11.07 (s, 1H), 8.53 |
| | | | (d, J = 13.9 Hz, 1H), 8.40-8.22 (m, 1H), 8.14 |
| | | | (d, J = 8.6 Hz, 1H), 8.05 (s, 1H), 7.59 (d, J = |
| | | | 8.7 Hz, 1H), 7.44-7.10 (m, 7H), 7.02-6.87 |
| | | | (m, 2H), 6.75-6.60 (m, 1H), 5.39-5.25 (m, |
| | | | 2H), 5.13-2.24 (m, 23H), 2.21-1.93 (m, |
| | | | 3H), 1.69-1.54 (m, 4H), 1.49-1.26 (m, 2H), |
| | | | 1.12-1.02 (m, 9H), 0.93-0.75 (m, 4H). |
| 1.15 | 1129.2 | [M − H]⁻ | (400 MHz, DMSO-d6) δ 11.11 (s, 1H), 8.65- |
| | | | 7.96 (m, 4H), 7.74-6.96 (m, 9H), 5.51-3.89 |
| | | | (m, 20H), 3.02-2.58 (m, 5H), 2.27-0.94 (m, |
| | | | 25H). |
| 1.16 | 1075.2 | [M − H]⁻ | (400 MHz, DMSO-d6) δ 11.14 (s, 1H), 8.60- |
| | | | 7.99 (m, 4H), 7.67-7.07 (m, 9H), 5.53-4.70 |
| | | | (m, 3H), 4.12 (m, 29H), 2.20-1.94 (m, 2H), |
| | | | 1.16-0.99 (m, 9H). |
| 1.17 | 1085.2 | [M − H]⁻ | (400 MHz, DMSO-d6) δ 11.08 (s, 1H), 8.66- |
| | | | 8.40 (m, 3H), 8.37-8.21 (m, 1H), 8.20- |
| | | | 7.99 (m, 2H), 7.92-7.73 (m, 1H), 7.58 (d, |
| | | | 1H), 7.49-7.28 (m, 1H), 7.09-6.92 (m, 2H), |
| | | | 6.84 (d, J = 7.7 Hz, 1H), 5.34 (dd, J = 12.9, 5.4 |
| | | | Hz, 1H), 4.82-4.70 (m, 1H), 4.53-4.08 (m, |
| | | | 4H), 3.92-0.77 (m, 40H). |
| 1.18 | 1085.2 | [M − H]⁻ | |
| 1.19 | 1111.3 | [M + H]⁺ | (400 MHz, Methanol-d4) δ 8.19 (m, 2H), 8.17- |
| | | | 8.10 (m, 1H), 8.04 (d, J = 8.6 Hz, 1H), 7.69 |
| | | | (d, J = 8.4 Hz, 1H), 7.49 (d, J = 7.4 Hz, 2H), |
| | | | 7.46-7.27 (m, 3H), 7.03 (d, J = 2.4 Hz, 2H), |
| | | | 6.95 (d, J = 8.1 Hz, 1H), 5.33 (m, 1H), 4.95 |
| | | | (d, J = 7.2 Hz, 1H), 4.69 (s, 1H), 4.60-4.29 |
| | | | (m, 4H), 4.29-4.04 (m, 1H), 4.04-3.86 (m, |
| | | | 2H), 3.86-3.64 (m, 3H), 3.56-3.39 (m, 3H), |
| | | | 3.27-3.10 (m, 1H), 2.95 (m, 2H), 2.90-2.75 |
| | | | (m, 2H), 2.65 (q, J = 9.9, 9.1 Hz, 3H), 2.42- |
| | | | 2.11 (m, 4H), 1.91 (m, 3H), 1.79-1.62 (m, |
| | | | 3H), 1.18 (s, 8H), 1.09 (s, 1H), 0.96 (d, J = 7.0 |
| | | | Hz, 1H), 0.83-0.41 (m, 1H). |
| 1.20 | 1092.1 | [M − H]⁻ | (400 MHz, DMSO-d6) δ 11.09 (s, 1H), 8.65- |
| | | | 6.99 (m, 10H), 5.44-0.84 (m, 46H). |
| 1.21 | 1110.7 | [M − H]⁻ | (400 MHz, DMSO-d6) δ 11.09 (s, 1H), 8.55 |
| | | | (s, 1H), 8.38 (d, J = 8.4 Hz, 1H), 8.14 (d, J = |
| | | | 8.5 Hz, 1H), 8.06 (s, 1H), 7.59 (d, J = 8.4 Hz, |
| | | | 1H), 7.50-7.34 (m, 5H), 7.01 (d, J = 8.6 Hz, |
| | | | 2H), 6.83 (s, 1H), 5.39-5.07 (m, 3H), 4.82- |
| | | | 4.68 (m, 2H), 4.54-3.82 (m, 11H), 2.97-2.56 |
| | | | (m, 8H), 2.22-1.94 (m, 4H), 1.78-1.49 (m, |
| | | | 4H), 1.20-0.91 (m, 12H). |
| 1.22 | 1087.2 | [M − H]⁻ | (400 MHz, DMSO) δ 11.08 (s, 1H), 8.59- |
| | | | 5.79 (m, 13H), 5.46-0.50 (m, 47H). |
| 1.23 | 1092.1 | [M − H]⁻ | (400 MHz, DMSO-d6) δ 11.12 (s, 1H), 8.60- |
| | | | 6.68 (m, 12H), 5.50-0.83 (m, 42H). |
| 1.24 | 1077.1 | [M + H]⁺ | (400 MHz, DMSO-d6) δ 11.14 (s, 1H), 8.69 |
| | | | (d, J = 11.8 Hz, 1H), 8.64-8.52 (m, 1H), 7.93 |
| | | | (d, J = 8.5 Hz, 1H), 7.73-7.56 (m, 1H), 7.51- |
| | | | 7.01 (m, 6H), 6.25-5.75 (m, 1H), 5.50- |
| | | | 5.37 (m, 1H), 5.34-5.03 (m, 1H), 4.75 (dd, J = |
| | | | 17.4, 8.5 Hz, 1H), 4.63-3.86 (m, 11H), |
| | | | 3.78-2.83 (m, 20H), 2.74 (s, 1H), 2.55 (d, J = |
| | | | 1.1 Hz, 1H), 2.15 (s, 2H), 1.22-0.99 (m, |
| | | | 9H). |
| 1.25 | 1074.1 | [M + H]⁺ | 1H NMR (400 MHz, Methanol-d4) δ 8.21 (d, |
| | | | J = 8.3 Hz, 2H), 8.19-8.08 (m, 1H), 8.03 (d, |
| | | | J = 8.6 Hz, 1H), 7.72 (d, J = 8.6 Hz, 1H), 7.27 |
| | | | (m, 5H), 7.12-6.81 (m, 4H), 5.55-5.16 (m, |
| | | | 2H), 4.92 (m, 6H), 4.72-4.31 (m, 2H), 4.31- |
| | | | 4.01 (m, 2H), 4.01 (s, 1H), 3.55-3.37 (m, |
| | | | 3H), 3.28-3.10 (m, 1H), 3.05 (m, 1H), 3.00- |
| | | | 2.89 (m, 1H), 2.81 (m, 2H), 2.29 (s, 1H), |
| | | | 2.25-2.01 (m, 3H), 1.96 (s, 1H), 1.90-1.54 |
| | | | (m, 7H), 1.31 (s, 1H), 1.20 (m, 9H), 1.12 (m, |
| | | | 3H). |
| 1.26 | 1103.2 | [M + H]⁺ | (400 MHz, DMSO-d6) δ 11.14 (s, 1H), 9.74 |
| | | | (s, 1H), 8.69 (d, J = 12.4 Hz, 1H), 8.62-8.47 |
| | | | (m, 1H), 7.92 (d, J = 8.5 Hz, 1H), 7.62 (t, J = |
| | | | 7.5 Hz, 1H), 7.47-7.28 (m, 4H), 7.26 (s, 2H), |
| | | | 6.25-5.75 (m, 1H), 5.59-5.36 (m, 1H), 5.5- |
| | | | 5.0 (m, 1H), 4.88-4.66 (m, 1H), 4.64-3.86 |
| | | | (m, 11H), 3.80-3.18 (m, 15H), 3.16-2.84 |
| | | | (m, 1H), 2.87-2.62 (m, 3H), 2.57-2.53 (m, |
| | | | 1H), 2.36-1.89 (m, 4H), 1.81-1.45 (m, 1H), |
| | | | 1.22-0.95 (m, 9H). |
| 1.27 | 1074.1 | [M − H]⁻ | (400 MHz, DMSO-d6) δ 11.10 (s, 1H), 8.65- |
| | | | 6.64 (m, 16H), 5.45-0.95 (m, 33H). |
| 1.28 | 1030.1 | [M − H]⁻ | (400 MHz, DMSO-d6) δ 11.12 (s, 1H), 8.83- |
| | | | 8.47 (m, 2H), 8.04-7.78 (m, 1H), 7.72-7.52 |
| | | | (m, 1H), 7.52-6.98 (m, 8H), 6.14-5.81 (m, |
| | | | 1H), 5.50-5.32 (m, 1H), 5.30-0.60 (m, |
| | | | 43H). |
| 1.29 | 1097.3 | [M − H]⁻ | (400 MHz, DMSO-d6) δ 11.13 (s, 1H), 9.96 |
| | | | (s, 1H), 8.57-8.17 (m, 2H), 8.09-7.79 (m, |
| | | | 2H), 7.54 (d, J = 8.5 Hz, 1H), 7.47-7.11 (m, |
| | | | 7H), 6.00-5.80 (m, 1H), 5.41 (dd, J = 12.8, |
| | | | 5.4 Hz, 1H), 5.29-5.02 (m, 1H), 4.74 (m, 1H), |
| | | | 4.59-4.48 (m, 1H), 4.44-3.70 (m, 10H), |
| | | | 3.09-2.54 (m, 2H), 2.38-1.95 (m, 3H), 1.74 |
| | | | (m, 4H), 1.06 (m, 9H). |
| 1.30 | 1069.5 | [M − H]⁻ | (400 MHz, DMSO-d6) δ 11.13 (s, 1H), 9.42 |
| | | | (s, 1H), 8.74-8.12 (m, 2H), 8.09-7.80 (m, |
| | | | 2H), 7.63-7.12 (m, 8H), 5.91-5.80 (m, 1H), |
| | | | 5.51-5.01 (m, 1H), 4.85-3.86 (m, 5H), 3.02- |
| | | | 2.57 (m, 3H), 2.33 (m, 1H), 2.23-1.95 (m, |
| | | | 4H), 1.76 (s, 1H), 1.06 (m, 9H). |
| 1.31 | 1097.7 | [M − H]⁻ | (400 MHz, DMSO-d6) δ 11.13 (d, J = 3.2 Hz, |
| | | | 1H), 9.4-9.1 (m, 1H), 8.60-8.45 (m, 1H), |
| | | | 8.37-8.18 (m, 1H), 8.04 (d, J = 8.5 Hz, 1H), |
| | | | 7.96 (s, 1H), 7.54 (d, J = 8.5 Hz, 1H), 7.47- |
| | | | 7.17 (m, 7H), 6.0-5.75 (m, 1H), 5.42 (dd, J = |
| | | | 12.8, 5.4 Hz, 1H), 5.36-5.01 (m, 1H), 4.93 |
| | | | -4.44 (m, 1H), 4.44-4.19 (m, 4H), 4.17- |
| | | | 3.18 (m, 24H), 3.17-2.83 (m, 2H), 2.83- |
| | | | 2.59 (m, 2H), 2.20-1.90 (m, 4H), 1.89-1.69 |
| | | | (m, 1H), 1.22-1.03 (m, 9H). |
| 1.32 | 1069.9 | [M − H]⁻ | (400 MHz, DMSO-d6) δ 11.13 (s, 1H), 10.21- |
| | | | 9.17 (m, 1H), 8.49 (d, J = 11.8 Hz, 1H), 8.40- |
| | | | 8.18 (m, 1H), 8.07-7.85 (m, 2H), 7.63- |
| | | | 7.04 (m, 7H), 5.98-5.77 (m, 1H), 5.76 (s, |
| | | | 1H), 5.51-5.04 (m, 2H), 4.81-3.66 (m, |
| | | | 14H), 3.33 (m, 10H), 3.02-2.54 (m, 3H), |
| | | | 2.37-1.69 (m, 6H), 1.17-0.89 (m, 9H). |
| 1.33 | 1083.7 | [M − H]⁻ | (400 MHz, DMSO-d6) δ 11.14 (s, 1H), 9.78 |
| | | | (s, 1H), 8.49 (d, J = 12.5 Hz, 1H), 8.35-8.25 |
| | | | (m, 1H), 8.04 (d, J = 8.5 Hz, 1H), 7.97 (s, 1H), |
| | | | 7.54 (d, J = 8.6 Hz, 1H), 7.50-7.18 (m, 6H), |
| | | | 6.0-5.75 (m, 1H), 5.43 (dd, J = 12.7, 5.4 Hz, |
| | | | 1H), 5.4-5.0 (m, 1H), 4.74 (dd, J = 16.0, 8.7 |
| | | | Hz, 1H), 4.59-3.84 (m, 10H), 3.83-3.04 (m, |
| | | | 18H), 3.03-2.62 (m, 4H), 2.39-1.95 (m, |
| | | | 3H), 1.75-1.25 (m, 1H), 1.2-0.9 (m, 9H). |
| 1.34 | 1057.7 | [M − H]⁻ | (400 MHz, DMSO-d6) δ 11.14 (s, 1H), 9.73 |
| | | | (s, 1H), 8.49 (d, J = 11.9 Hz, 1H), 8.35-8.25 |
| | | | (m, 1H), 8.04 (d, J = 8.5 Hz, 1H), 7.97 (s, 1H), |
| | | | 7.54 (d, J = 8.5 Hz, 1H), 7.50-7.04 (m, 8H), |
| | | | 6.0-5.75 (m, 1H), 5.42 (d, J = 13.0 Hz, 1H), |
| | | | 5.34-4.95 (m, 1H), 4.85-3.79 (m, 12H), |
| | | | 3.75-3.2 (m, 15H), 3.03-2.62 (m, 3H), 2.20- |
| | | | 1.88 (m, 3H), 1.20-0.99 (m, 9H). |
| 1.35 | 1083.4 | [M − H]⁻ | (400 MHz, DMSO-d6) δ 11.13 (s, 1H), 10.06 |
| | | | (m, 1H), 8.49 (d, J = 11.3 Hz, 1H), 8.29 (m, |
| | | | 1H), 8.09-7.79 (m, 2H), 7.54 (d, J = 8.5 Hz, |
| | | | 1H), 7.48-7.04 (m, 9H), 5.91 (d, J = 8.0 Hz, |
| | | | 1H), 5.80 (d, J = 8.0 Hz, 1H), 5.45-4.97 (m, |
| | | | 2H), 4.83-4.47 (m, 1H), 4.45-3.74 (m, |
| | | | 10H), 3.00-2.53 (m, 5H), 2.06 (m, 1H), 1.76 |
| | | | (s, 1H), 1.20-0.93 (m, 9H). |
| 1.36 | 1069.4 | [M − H]⁻ | (400 MHz, DMSO-d6) δ 11.13 (d, J = 3.2 Hz, |
| | | | 1H), 9.7-9.3 (m, 1H), 8.60-8.45 (m, 1H), |
| | | | 8.37-8.18 (m, 1H), 8.04 (d, J = 8.5 Hz, 1H), |
| | | | 7.96 (s, 1H), 7.54 (d, J = 8.5 Hz, 1H), 7.47- |
| | | | 7.17 (m, 7H), 6.0-5.75 (m, 1H), 5.62-5.36 |
| | | | (m, 1H), 5.35-5.0 (m, 1H), 4.86-3.82 (m, |
| | | | 12H), 3.8-3.05 (m, 16H), 3.02-2.60 (m, 2H), |
| | | | 2.25-1.8 (m, 4H), 1.25-0.9 (m, 9H). |
| 1.37 | 1083.3 | [M − H]⁻ | (400 MHz, DMSO-d6) δ 11.14 (s, 1H), 9.78 |
| | | | (s, 1H), 8.49 (m, 1H), 8.36-8.20 (m, 1H), |
| | | | 8.04 (d, J = 8.4 Hz, 1H), 7.97 (s, 1H), 7.54 (d, |
| | | | J = 8.5 Hz, 1H), 7.47-7.18 (m, 7H), 5.95- |
| | | | 5.78 (m, 1H), 5.76 (s, 3H), 5.43 (dd, J = 12.7, |
| | | | 5.4 Hz, 1Hr), 5.40-5.0 (m, 1H), 4.74 (m, 1H), |
| | | | 4.59-3.81 (m, 10H), 3.71 (s, 1H), 3.33 (m, |
| | | | 10H), 3.01-2.54 (m, 4H), 2.42-1.84 (m, |
| | | | 5H), 1.85-1.56 (m, 1H), 1.24 (s, 1H), 1.22- |
| | | | 0.95 (m, 9H). |
| 1.38 | 1069.7 | [M − H]⁻ | (400 MHz, DMSO-d6) δ 11.13 (s, 1H), 10- |
| | | | 9.69 (m, 1H), 8.48 (d, J = 13.8 Hz, 1H), 8.4- |
| | | | 8.2 (m, 1H), 8.14-7.76 (m, 2H), 7.61-6.99 |
| | | | (m, 7H), 6.0-5.75 (m, 1H), 5.60-3.05 (m, |
| | | | 30H), 3.03-2.58 (m, 3H), 2.30-1.87 (m, |
| | | | 4H), 1.33-0.82 (m, 9H). |
| 1.39 | 1143.7 | [M − H]⁻ | 1H NMR (400 MHz, DMSO) δ 11.07 (s, 1H), |
| | | | 8.53 (d, J = 11.5 Hz, 1H), 8.33 (m, 1H), 8.17- |
| | | | 8.02 (m, 2H), 7.59 (d, J = 8.7 Hz, 1H), 7.36 |
| | | | (m, 5H), 6.98-6.76 (m, 2H), 6.67 (d, J = 8.6 |
| | | | Hz, 1H), 5.30 (dd, J = 12.4, 5.5 Hz, 1H), 4.74 |
| | | | (m, 1H), 4.59-3.84 (m, 12H), 3.76 (m, 8H), |
| | | | 3.35-3.20 (m, 7H), 3.20-2.55 (m, 4H), 2.38- |
| | | | 1.93 (m, 5H), 1.87-1.66 (m, 2H), 1.25 (m, |
| | | | 1H), 1.06 (m, 10H), 0.07 (s, 1H). |
| 1.40 | 1099.2 | [M + H]⁺ | (400 MHz, DMSO-d6) δ 11.13 (s, 1H), 9.88 |
| | | | (s, 1H), 8.49 (d, J = 11.6 Hz, 1H), 8.35-8.19 |
| | | | (m, 1H), 8.04 (d, J = 8.5 Hz, 1H), 7.97 (s, 1H), |
| | | | 7.54 (d, J = 8.5 Hz, 1H), 7.48-7.25 (m, 5H), |
| | | | 7.21 (d, J = 3.9 Hz, 2H), 6.0-5.75 (m, 1H), |
| | | | 5.42 (dd, J = 12.8, 5.4 Hz, 1H), 5.35-4.97 |
| | | | (m, 1H), 4.89-4.66 (m, 1H), 4.62-3.07 (m, |
| | | | 29H), 3.05-2.79 (m, 1H), 2.79-2.56 (m, |
| | | | 2H), 2.23-1.92 (m, 3H), 1.92-1.77 (m, 2H), |
| | | | 1.23-0.96 (m, 9H). |
| 1.41 | 1083.1 | [M − H]⁻ | (400 MHz, DMSO-d6) δ 11.17 (s, 1H), 8.60- |
| | | | 8.25 (m, 3H), 8.12-7.94 (m, 2H), 7.62-7.13 |
| | | | (m, 8H), 6.02-0.98 (m, 46H). |
| 1.42 | 1099.2 | [M − H]⁻ | (400 MHz, DMSO-d6) δ 11.13 (s, 1H), 9.83 |
| | | | (s, 1H), 8.49 (d, J = 11.7 Hz, 1H), 8.35-8.23 |
| | | | (m, 1H), 8.14-7.85 (m, 2H), 7.54 (d, J = 8.6 |
| | | | Hz, 1H), 7.47-7.24 (m, 5H), 7.24-7.06 (m, |
| | | | 2H), 6.0-5.75 (m, 1H), 5.42 (d, J = 11.7 Hz, |
| | | | 1H), 5.34-5.00 (m, 1H), 4.92-4.64 (m, 1H), |
| | | | 4.57-3.15 (m, 29H), 3.14-2.58 (m, 2H), |
| | | | 2.19-1.78 (m, 4H), 1.38-0.93 (m, 9H). |
| 1.43 | 1083.6 | [M − H]⁻ | (400 MHz, DMSO-d6) δ 11.14 (s, 1H), 9.83 |
| | | | (s, 1H), 8.49 (d, J = 12.8 Hz, 1H), 8.37-8.25 |
| | | | (m, 1H), 8.04 (d, J = 8.5 Hz, 1H), 7.97 (s, 1H), |
| | | | 7.54 (d, J = 8.5 Hz, 1H), 7.48-7.27 (m, 5H), |
| | | | 7.21 (d, J = 9.0 Hz, 2H), 6.0-5.75 (m, 1H), |
| | | | 5.42 (dd, J = 12.7, 5.4 Hz, 1H), 5.19 (d, J = |
| | | | 88.6 Hz, 1H), 4.75 (s, 1H), 4.60-3.0 (m, |
| | | | 28H), 2.99-2.57 (m, 3H), 2.45-1.97 (m, |
| | | | 4H), 1.23-0.98 (m, 9H). |
| 1.44 | 1075.2 | [M − H]⁻ | (400 MHz, DMSO-d6) δ 11.10 (s, 1H), 8.65- |
| | | | 8.40 (m, 2H), 8.14 (d, J = 8.5 Hz, 1H), 8.07 |
| | | | (d, J = 7.6 Hz, 1H), 7.61 (d, J = 8.7 Hz, 1H), |
| | | | 7.46 (d, J = 8.1 Hz, 1H), 7.43-7.21 (m, 3H), |
| | | | 7.03-6.91 (m, 2H), 6.91-6.77 (m, 1H), 5.61- |
| | | | 5.12 (m, 2H), 4.83-4.43 (m, 3H), 4.42- |
| | | | 4.22 (m, 2H), 4.21-3.86 (m, 2H), 3.81-2.96 |
| | | | (m, 19H), 2.99-2.50 (m, 6H), 2.40 (s, 1H), |
| | | | 2.16-1.90 (m, 1H), 1.92-1.72 (m, 1H), 1.72- |
| | | | 1.50 (m, 2H), 1.19-0.88 (m, 9H). |
| 1.45 | 1074.5 | [M − H]⁻ | (400 MHz, DMSO-d6) δ 11.09 (s, 1H), 8.73- |
| | | | 8.52 (m, 2H), 7.93 (d, J = 8.5 Hz, 1H), 7.61 (t, |
| | | | J = 7.5 Hz, 1H), 7.48-7.26 (m, 5H), 7.06-6.96 |
| | | | (m, 2H), 6.84 (s, 1H), 6.13-5.93 (m, 1H), 5.38- |
| | | | 5.26 (m, 1H), 4.83-4.68 (m, 1H), 4.39-4.22 |
| | | | (m, 5H), 4.18-3.78 (m, 9H), 3.09-2.58 (m, |
| | | | 10H), 2.23-1.94 (m, 4H), 1.81-1.45 (m, |
| | | | 4H), 1.14-0.90 (m, 12H). |
| 1.46 | 1048.1 | [M − H]⁻ | 1H NMR (400 MHz, DMSO-d6) δ 11.12 (s, |
| | | | 1H), 8.86-8.51 (m, 2H), 8.01-7.85 (m, 1H), |
| | | | 7.60-7.04 (m, 9H), 5.48-5.32 (m, 1H), 5.30- |
| | | | 4.89 (m, 1H), 4.85-0.77 (m, 42H). |

### Example 2.1: ((2-(((2S)-1-((2S,3R)-3-((1-((1-(2,6-dioxopiperidin-3-yl)-3-methyl-2-oxo-2,3-dihydro-1H-benzo[d]imidazol-5-yl)methyl)piperidin-4-yl)methyl)-2-((R)-2-phenylmorpholine-4-carbonyl)pyrrolidin-1-yl)-3,3-dimethyl-1-oxobutan-2-yl)carbamoyl)benzo[b]thiophen-5-yl)difluoromethyl)phosphonic acid

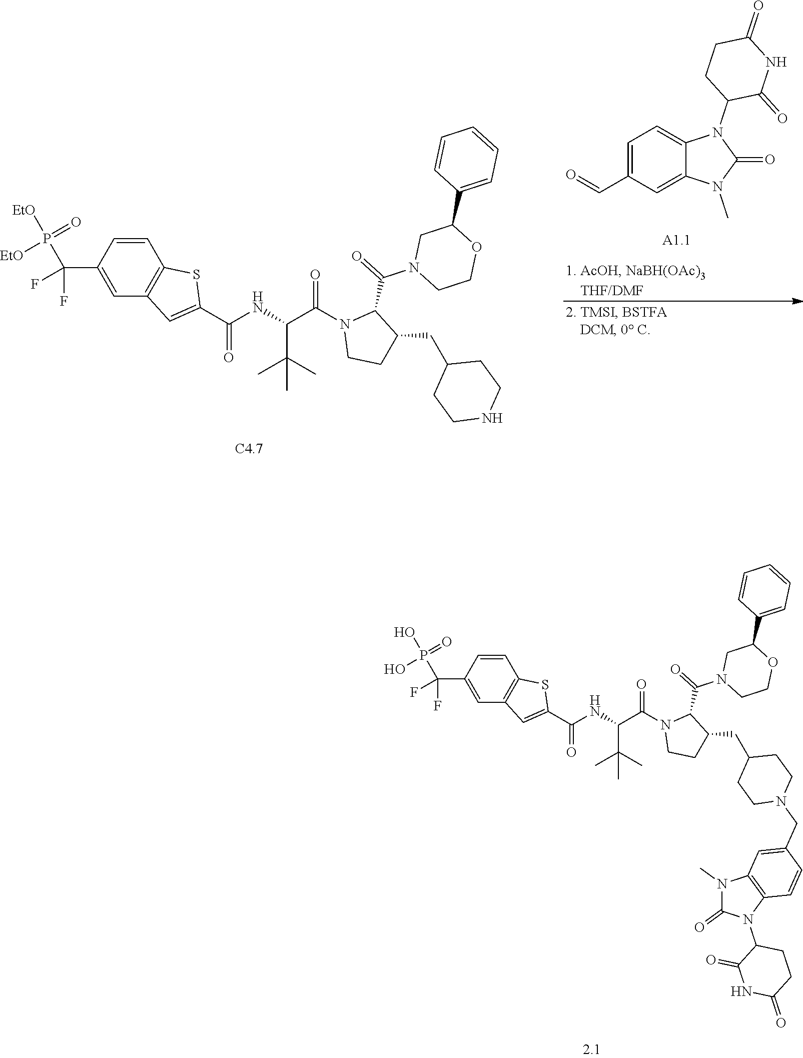 [SMILES: CCOP(=O)(OCC)C(F)(F)c1ccc2sc(C(=O)N[C@H](C(=O)N3CC[C@@H](CC4CCNCC4)[C@H]3C(=O)N3CCO[C@H](c4ccccc4)C3)C(C)(C)C)cc2c1 ; Cn1c(=O)n(C2CCC(=O)NC2=O)c2ccc(C=O)cc21 ; Cn1c(=O)n(C2CCC(=O)NC2=O)c2ccc(CN3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21]

AcOH (0.02 mL, 0.35 mmol) was added to a mixture of diethyl ((2-(((S)-3,3-dimethyl-1-oxo-1-((2S,3R)-2-((R)-2-phenylmorpholine-4-carbonyl)-3-(piperidin-4-ylmethyl)pyrrolidin-1-yl)butan-2-yl)carbamoyl)benzo[b]thiophen-5-yl)difluoromethyl)phosphonate Cd-7 (95 mg, 0.12 mmol) and 1-(2,6-dioxo-3-piperidyl)-3-methyl-2-oxo-benzimidazole-5-carbaldehyde A1.1 (57 mg, 0.2 mmol) in THF/DMF (3:1, 4 mL). After stirring for 1 hour, sodium triacetoxyborohydride (111 mg, 0.52 mmol) was added. After stirring overnight, the mixture was diluted with EtOAc and washed with sat. aq. NaHCO₃. The organic layer was dried over MgSO4, filtered, concentrated, and purified by silica gel flash column chromatography. The purified residue was resuspended in DCM and N,O-Bis(trimethylsilyl)trifluoroacetamide (0.28 mL, 1.1 mmol) and TMSI (1.0M in DCM, 0.35 mL, 0.35 mmol) were added at 0° C. After 30 minutes, the mixture was concentrated. Purification by reverse phase HPLC afforded ((2-(((2S)-1-((2S,3R)-3-((1-((1-(2,6-dioxopiperidin-3-yl)-3-methyl-2-oxo-2,3-dihydro-1H-benzo[d]imidazol-5-yl)methyl)piperidin-4-yl)methyl)-2-((R)-2-phenylmorpholine-4-carbonyl)pyrrolidin-1-yl)-3,3-dimethyl-1-oxobutan-2-yl)carbamoyl)benzo[b]thiophen-5-yl)difluoromethyl)phosphonic acid (2.1).

The compounds in Table 2.1 were prepared analogously to Example 2.1 modified as described in Table 2.2

TABLE 2.1
Examples 2.2-2.5

| Ex. | Name | Structure |
|---|---|---|
| 2.2 | ((2-(((2S)-3,3-dimethyl-1-((2S,3R)- 3-((1-(2-(3-methyl-1-(1-methyl-2,6- dioxopiperidin-3-yl)-2-oxo-2,3- dihydro-1H-benzo[d]imidazol-5- yl)ethyl)piperidin-4-yl)methyl)-2- ((R)-2-phenylmorpholine-4- carbonyl)pyrrolidin-1-yl)-1- oxobutan-2- yl)carbamoyl)benzo[b]thiophen-5- yl)difluoromethyl)phosphonic acid | 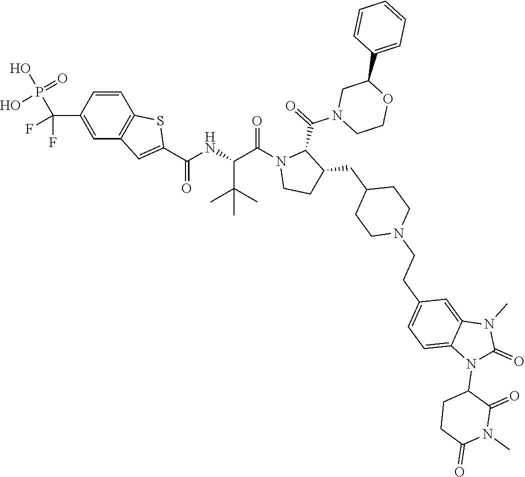 [SMILES: CN1C(=O)CCC(n2c(=O)n(C)c3cc(CCN4CCC(C[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)ccc32)C1=O] |
| 2.3 | ((2-(((2S)-1-((2S,3R)-3-((1-((1-(2,6- dioxopiperidin-3-yl)-3-methyl-2- oxo-2,3-dihydro-1H- benzo[d]imidazol-5- yl)methyl)azetidin-3-yl)methyl)-2- ((R)-2-phenylmorpholine-4- carbonyl)pyrrolidin-1-yl)-3,3- dimethyl-1-oxobutan-2- yl)carbamoyl)benzo[b]thiophen-5- yl)difluoromethyl)phosphonic acid | 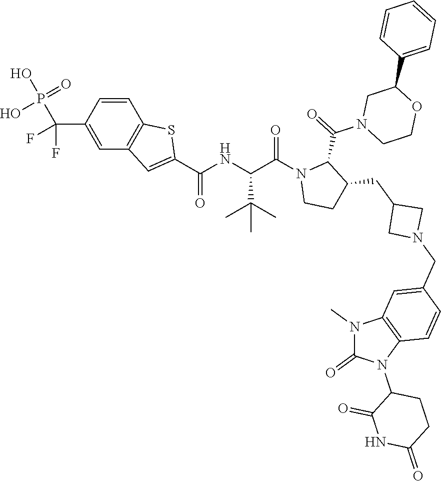 [SMILES: Cn1c(=O)n(C2CCC(=O)NC2=O)c2ccc(CN3CC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)C3)cc21] |
| 2.4 | ((2-(((2S)-1-((2S,3R)-3-((1-((1-(2,6- dioxopiperidin-3-yl)-4,6-difluoro-3- methyl-2-oxo-2,3-dihydro-1H- benzo[d]imidazol-5- yl)methyl)piperidin-4-yl)methyl)-2- ((R)-2-phenylmorpholine-4- carbonyl)pyrrolidin-1-yl)-3,3- dimethyl-1-oxobutan-2- yl)carbamoyl)benzo[b]thiophen-5- yl)difluoromethyl)phosphonic acid | 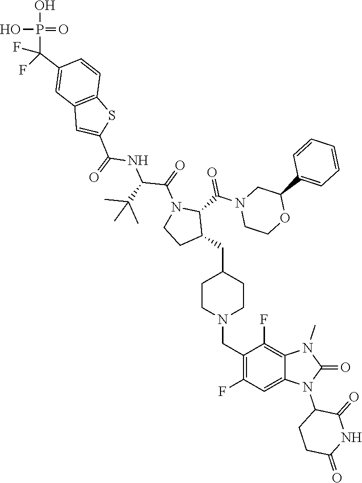 [SMILES: Cn1c(=O)n(C2CCC(=O)NC2=O)c2cc(F)c(CN3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)c(F)c21] |
| 2.5 | ((2-(((2S)-1-((2S,3R)-3-((1-((1-(2,6- dioxopiperidin-3-yl)-4-fluoro-3- methyl-2-oxo-2,3-dihydro-1H- benzo[d]imidazol-5- yl)methyl)piperidin-4-yl)methyl)-2- ((R)-2-phenylmorpholine-4- carbonyl)pyrrolidin-1-yl)-3,3- dimethyl-1-oxobutan-2- yl)carbamoyl)benzo[b]thiophen-5- yl)difluoromethyl)phosphonic acid | 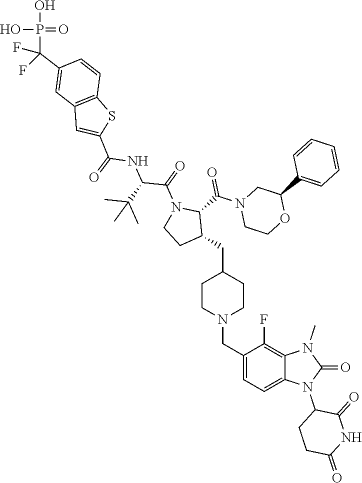 [SMILES: Cn1c(=O)n(C2CCC(=O)NC2=O)c2ccc(CN3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)c(F)c21] |

TABLE 2.2
Procedures and analogs to Example 2.1 Synthesis.

| | | | |
|---|---|---|---|
| Example | Starting | Instead of | Variation from |
| Number | Material | A1.1 | Example 2.1 |

| | | |
|---|---|---|
| 2.2 | C4.7 | A5.1 |
| 2.3 | E2.3 | A1.1 |
| 2.4 | C4.7 | A22.1 |
| 2.5 | C4.7 | A23.1 |

TABLE 2.3
ES/MS and 1H NMR data for Examples 2.1 to 2.5

| Example | ES/MS | | |
|---|---|---|---|
| Number | m/z | Ion | 1H NMR |
| 2.1 | 1029.8 | [M − H]⁻ | (400 MHz, DMSO-d6) δ 11.16 (s, 1H), 8.65- |
| | | | 8.31 (m, 2H), 8.18-7.98 (m, 2H), 7.72-7.58 |
| | | | (m, 1H), 7.54-7.06 (m, 7H), 6.63-6.49 (m, |
| | | | 1H), 5.54-5.22 (m, 2H), 5.10-0.93 (m, |
| | | | 42H). |
| 2.2 | 1043.8 | [M − H]⁻ | (400 MHz, DMSO-d6) δ 11.13 (s, 1H), 8.66- |
| | | | 8.55 (m, 1H), 8.55-8.31 (m, 1H), 8.17 (d, |
| | | | J = 8.9 Hz, 1H), 8.09 (s, 1H), 7.63 (d, J = 8.6 |
| | | | Hz, 1H), 7.54-6.92 (m, 8H), 5.45-5.35 (m, |
| | | | 1H), 5.33-5.03 (m, 1H), 4.86-4.68 (m, |
| | | | 2H), 4.55-0.95 (m, 42H). |
| 2.3 | 1002.1 | [M − H]⁻ | (400 MHz, DMSO-d6) δ 11.12 (s, 1H), 8.62- |
| | | | 8.35 (m, 2H), 8.17-7.99 (m, 2H), 7.66 (d, |
| | | | J = 8.6 Hz, 1H), 7.53-6.92 (m, 8H), 6.38- |
| | | | 0.59 (m, 40H). |
| 2.4 | 1066.7 | [M − H]⁻ | (400 MHz, DMSO-d6) δ 11.16 (s, 1H), 9.46- |
| | | | 9.28 (m, 1H), 8.60-8.29 (m, 2H), 8.13 (d, |
| | | | J = 8.7 Hz, 1H), 8.06 (s, 1H), 7.60 (d, J = 8.6 |
| | | | Hz, 1H), 7.49-7.26 (m, 7H), 5.48-5.40 (m, |
| | | | 1H), 4.81-4.60 (m, 1H), 4.51-4.26 (m, |
| | | | 4H), 4.24-3.74 (m, 5H), 3.06-2.58 (m, |
| | | | 9H), 2.10-1.79 (m, 7H), 1.68-1.51 (m, |
| | | | 1H), 1.49-1.23 (m, 4H), 1.18-0.96 (m, |
| | | | 12H). |
| 2.5 | 1048.7 | [M − H]⁻ | (400 MHz, DMSO-d6) δ 11.16 (s, 1H), 9.20 |
| | | | (s, 1H), 8.59-8.50 (m, 1H), 8.48-8.31 (m, |
| | | | 1H), 8.19-8.01 (m, 2H), 7.60 (d, J = 8.6 Hz, |
| | | | 1H), 7.48-7.27 (m, 6H), 7.27-7.07 (m, |
| | | | 2H), 5.51-5.37 (m, 1H), 5.27-4.97 (m, 1H), |
| | | | 4.80-4.62 (m, 2H), 4.50-4.26 (m, 5H), 4.19- |
| | | | 3.74 (m, 4H), 3.17-2.58 (m, 8H), 2.10-1.78 |
| | | | (m, 6H), 1.60-1.17 (m, 5H), 1.15-0.96 (m, |
| | | | 12H). |

### Example 3.1: ((2-(((2S)-1-((2S,3R)-3-(2-(4-((1-(2,6-dioxopiperidin-3-yl)-3-methyl-2-oxo-2,3-dihydro-1H-benzo[d]imidazol-5-yl)methyl)piperidin-1-yl)-2-oxoethoxy)-2-((R)-2-phenylmorpholine-4-carbonyl)pyrrolidin-1-yl)-3,3-dimethyl-1-oxobutan-2-yl)carbamoyl)benzo[b]thiophen-5-yl)fluoromethyl)phosphonic acid

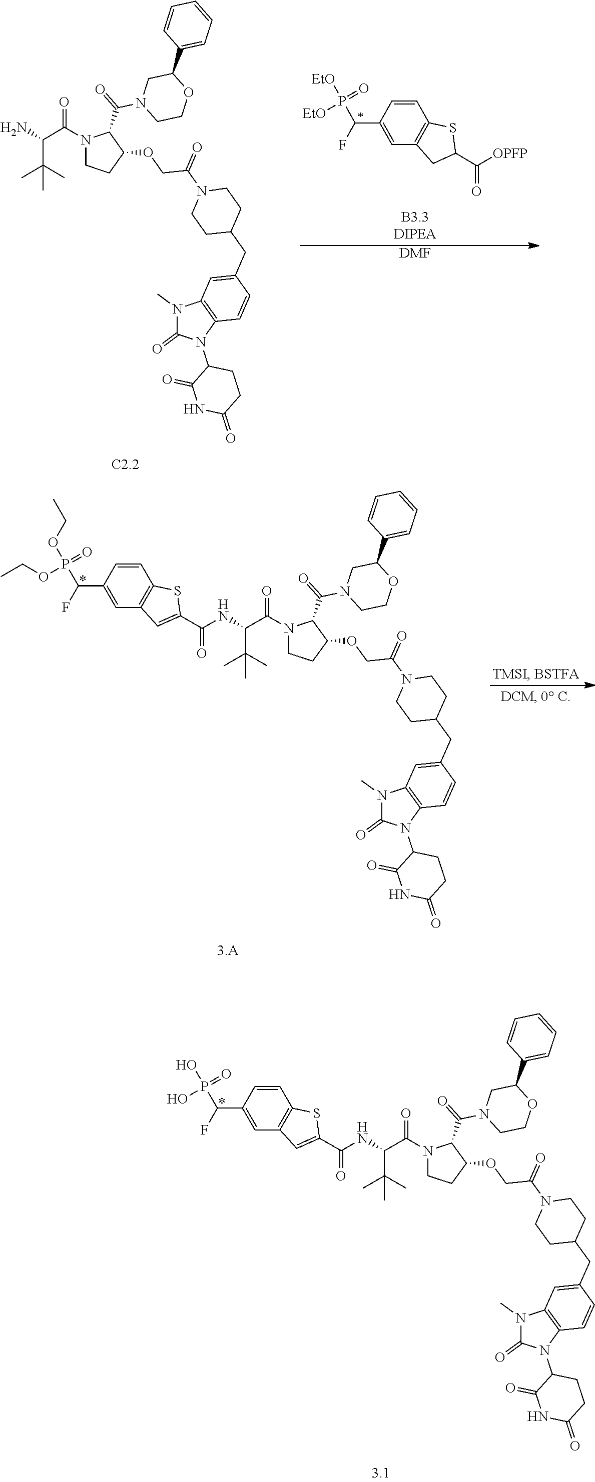

Step 1: To a solution of B3.3 (30 mg, 0.059 mmol, 1.0 equiv.) in DMF (2.0 mL) was added DIPEA (0.10 mL, 0.586 mmol, 10.0 equiv.) followed by a solution of 3-(5-((1-(2-(((2S,3R)-1-((S)-2-amino-3,3-dimethylbutanoyl)-2-((R)-2-phenylmorpholine-4-carbonyl)pyrrolidin-3-yl)oxy)acetyl)piperidin-4-yl)methyl)-3-methyl-2-oxo-2,3-dihydro-1H-benzo[d]imidazol-1-yl)piperidine-2,6-dione 2,2,2-trifluoroacetate (60 mg, 0.067 mmol, 1.14 equiv.) in DMF (1.5 mL). The solution was stirred at room temperature for 18 min and purified directly by silica gel chromatography (0-100% EtOAc in hexanes then 0-20% MeOH in EtOAc) to afford diethyl ((2-(((2S)-1-((2S,3R)-3-(2-(4-((1-(2,6-dioxopiperidin-3-yl)-3-methyl-2-oxo-2,3-dihydro-1H-benzo[d]imidazol-5-yl)methyl)piperidin-1-yl)-2-oxoethoxy)-2-((R)-2-phenylmorpholine-4-carbonyl)pyrrolidin-1-yl)-3,3-dimethyl-1-oxobutan-2-yl)carbamoyl)benzo[b]thiophen-5-yl)fluoromethyl)phosphonate 3.A. ES/MS: m/z=1112.8 [M−H]⁻.

Step 2: To a solution of 3.A (60 mg, 0.054 mmol, 1.0 equiv.) in DCM (6.0 mL) cooled in an ice bath was added BSTFA (0.26 mL, 0.97 mmol, 18.0 equiv.) followed by iodotrimethylsilane (1 M in DCM, 0.48 mL, 0.48 mmol, 9.0 equiv.). The solution was stirred for 20 min prior to concentrating and purifying by prep-HPLC (10-90% ACN (0.1% TEA) in water (0.1% TEA)) to afford the title compound (3.1).

The compounds in Table 3.1 were prepared analogously to Example 3.1 modified as described in Table 3.2

TABLE 3.1
Examples 3.2 to 3.14

| Ex. | Name | Structure |
|---|---|---|
| 3.2 | ((1S)-(2-(((2S)-1- ((2S,3R)-3-(2-(4-((1- (2,6-dioxopiperidin-3- yl)-3-methyl-2-oxo-2,3- dihydro-1H- benzo[d]imidazol-5- yl)methyl)piperidin-1- yl)-2-oxoethoxy)-2- ((R)-2- phenylmorpholine-4- carbonyl)pyrrolidin-1- yl)-3,3-dimethyl-1- oxobutan-2- yl)carbamoyl)benzo[b] thiophen-5- yl)fluoromethyl) phosphonic acid or ((1R)-(2- (((2S)-1-((2S,3R)-3-(2- (4-((1-(2,6- dioxopiperidin-3-yl)-3- methyl-2-oxo-2,3- dihydro-1H- benzo[d]imidazol-5- yl)methyl)piperidin-1- yl)-2-oxoethoxy)-2- ((R)-2- phenylmorpholine-4- carbonyl)pyrrolidin-1- yl)-3,3-dimethyl-1- oxobutan-2- yl)carbamoyl)benzo[b] thiophen-5- yl)fluoromethyl) phosphonic acid | 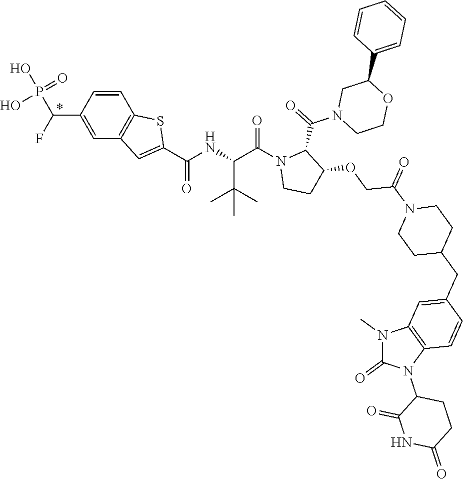 [SMILES: Cn1c(=O)n(C2CCC(=O)NC2=O)c2ccc(CC3CCN(C(=O)CO[C@@H]4CCN(C(=O)[C@@H](NC(=O)c5cc6cc(C(F)P(=O)(O)O)ccc6s5)C(C)(C)C)[C@@H]4C(=O)N4CCO[C@H](c5ccccc5)C4)CC3)cc21] |
| 3.3 | ((1S)-(2-(((2S)-1- ((2S,3R)-3-((1-((1-(2,6- dioxopiperidin-3-yl)-3- methyl-2-oxo-2,3- dihydro-1H- benzo[d]imidazol-5- yl)methyl)piperidin-4- yl)methyl)-2-((R)-2- phenylmorpholine-4- carbonyl)pyrrolidin-1- yl)-3,3-dimethyl-1- oxobutan-2- yl)carbamoyl)benzo[b] thiophen-5- yl)fluoromethyl) phosphonic acid or ((1R)-(2- (((2S)-1-((2S,3R)-3-((1- ((1-(2,6-dioxopiperidin- 3-yl)-3-methyl-2-oxo- 2,3-dihydro-1H- benzo[d]imidazol-5- yl)methyl)piperidin-4- yl)methyl)-2-((R)-2- phenylmorpholine-4- carbonyl)pyrrolidin-1- yl)-3,3-dimethyl-1- oxobutan-2- yl)carbamoyl)benzo[b] thiophen-5- yl)fluoromethyl) phosphonic acid | 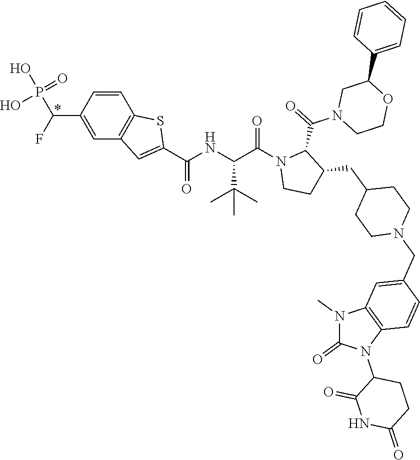 [SMILES: Cn1c(=O)n(C2CCC(=O)NC2=O)c2ccc(CN3CCC(C[C@@H]4CCN(C(=O)[C@@H](NC(=O)c5cc6cc(C(F)P(=O)(O)O)ccc6s5)C(C)(C)C)[C@@H]4C(=O)N4CCO[C@H](c5ccccc5)C4)CC3)cc21] |
| 3.4 | ((2-(((2S)-1-((2S,3R)-3- ((4-((1-(2,6- dioxopiperidin-3-yl)-3- methyl-2-oxo-2,3- dihydro-1H- benzo[d]imidazol-5- yl)methyl)piperidine-1- carbonyl)oxy)-2-((R)-2- phenylmorpholine-4- carbonyl)pyrrolidin-1- yl)-3,3-dimethyl-1- oxobutan-2- yl)carbamoyl)benzo[b] thiophen-5- yl)difluoromethyl) phosphonic acid | 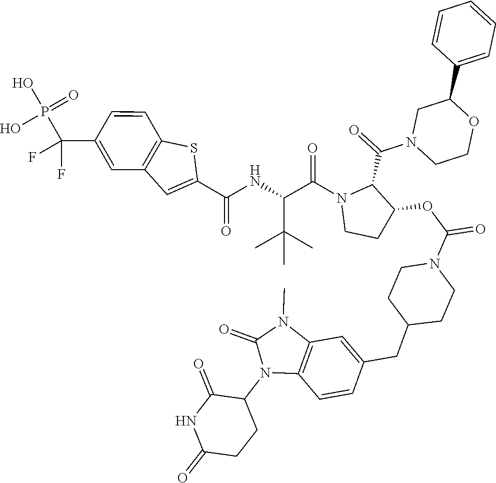 [SMILES: Cn1c(=O)n(C2CCC(=O)NC2=O)c2ccc(CC3CCN(C(=O)O[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21] |
| 3.5 | ((2-(((2S)-1-((2S,3S)-3- ((4-((1-(2,6- dioxopiperidin-3-yl)-3- methyl-2-oxo-2,3- dihydro-1H- benzo[d]imidazol-5- yl)methyl)piperidin-1- yl)methyl)-2-((R)-2- phenylmorpholine-4- carbonyl)pyrrolidin-1- yl)-3,3-dimethyl-1- oxobutan-2- yl)carbamoyl)benzo[b] thiophen-5- yl)difluoromethyl) phosphonic acid | 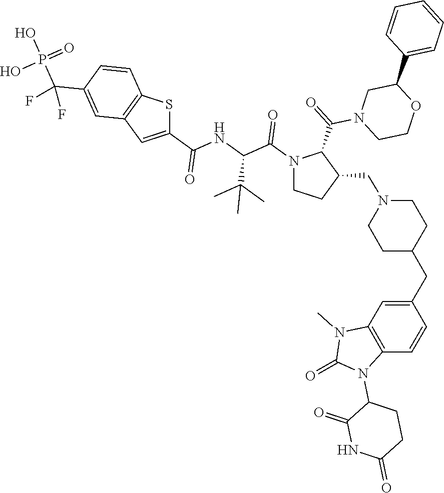 [SMILES: Cn1c(=O)n(C2CCC(=O)NC2=O)c2ccc(CC3CCN(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21] |
| 3.6 | ((2-(((2S)-1-((2S,3R)-3- ((1-((1-(2,6- dioxopiperidin-3-yl)-3- methyl-2-oxo-2,3- dihydro-1H- benzo[d]imidazol-5- yl)methyl)piperidin-4- yl)oxy)-2-((R)-2- phenylmorpholine-4- carbonyl)pyrrolidin-1- yl)-3,3-dimethyl-1- oxobutan-2- yl)carbamoyl)benzo[b] thiophen-5- yl)difluoromethyl) phosphonic acid | 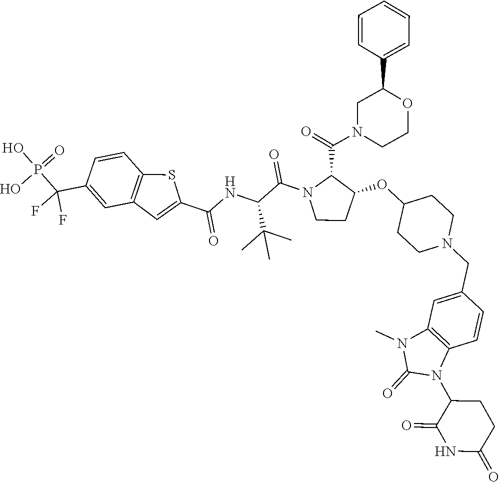 [SMILES: Cn1c(=O)n(C2CCC(=O)NC2=O)c2ccc(CN3CCC(O[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21] |
| 3.7 | ((2-(((2S)-1-((2S,3R)-3- (((3S)-1-((1-(2,6- dioxopiperidin-3-yl)-3- methyl-2-oxo-2,3- dihydro-1H- benzo[d]imidazol-5- yl)methyl)pyrrolidin-3- yl)methyl)-2-((R)-2- phenylmorpholine-4- carbonyl)pyrrolidin-1- yl)-3,3-dimethyl-1- oxobutan-2- yl)carbamoyl)benzo[b] thiophen-5- yl)difluoromethyl) phosphonic acid | 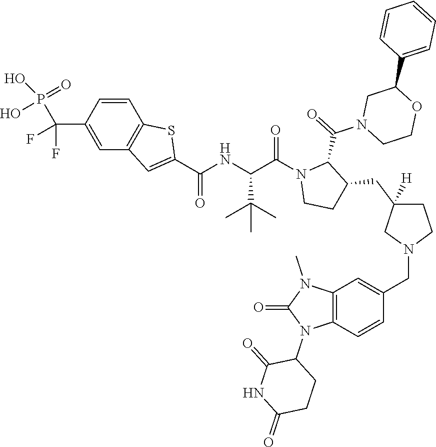 [SMILES: Cn1c(=O)n(C2CCC(=O)NC2=O)c2ccc(CN3CC[C@H](C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)C3)cc21] |
| 3.8 | ((2-(((2S)-1-((2S,3S)-3- (1-((1-(2,6- dioxopiperidin-3-yl)-3- methyl-2-oxo-2,3- dihydro-1H- benzo[d]imidazol-5- yl)methyl)azetidin-3- yl)-2-((R)-2- phenylmorpholine-4- carbonyl)pyrrolidin-1- yl)-3,3-dimethyl-1- oxobutan-2- yl)carbamoyl)benzo[b] thiophen-5- yl)difluoromethyl) phosphonic acid | 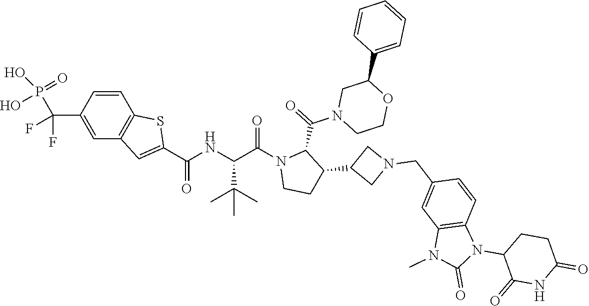 [SMILES: Cn1c(=O)n(C2CCC(=O)NC2=O)c2ccc(CN3CC([C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)C3)cc21] |
| 3.9 | ((2-(((2S)-1-((2S,3R)-3- (2-(1-((1-(2,6- dioxopiperidin-3-yl)-3- methyl-2-oxo-2,3- dihydro-1H- benzo[d]imidazol-5- yl)methyl)piperidin-4- yl)ethyl)-2-((R)-2- phenylmorpholine-4- carbonyl)pyrrolidin-1- yl)-3,3-dimethyl-1- oxobutan-2- yl)carbamoyl)benzo[b] thiophen-5- yl)difluoromethyl) phosphonic acid | 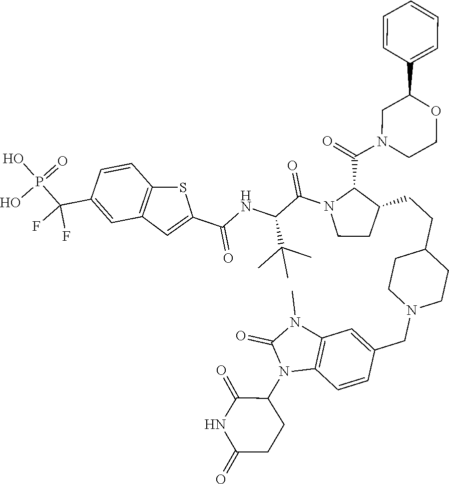 [SMILES: Cn1c(=O)n(C2CCC(=O)NC2=O)c2ccc(CN3CCC(CC[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21] |
| 3.10 | ((2-(((2S)-1-((2S,3R)-3- (((3R)-1-((1-(2,6- dioxopiperidin-3-yl)-3- methyl-2-oxo-2,3- dihydro-1H- benzo[d]imidazol-5- yl)methyl)pyrrolidin-3- yl)methyl)-2-((R)-2- phenylmorpholine-4- carbonyl)pyrrolidin-1- yl)-3,3-dimethyl-1- oxobutan-2- yl)carbamoyl)benzo[b] thiophen-5- yl)difluoromethyl) phosphonic acid | 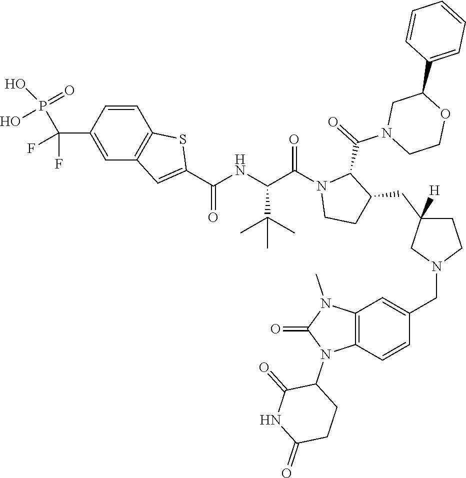 [SMILES: Cn1c(=O)n(C2CCC(=O)NC2=O)c2ccc(CN3CC[C@@H](C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)C3)cc21] |
| 3.11 | ((1S)-(2-(((2S)-1- ((2S,3R)-2-((6S,7R)-7- cyano-6-phenyl-4- azaspiro[2.4]heptane-4- carbonyl)-3-(2-(4-((1- (2,6-dioxopiperidin-3- yl)-3-methyl-2-oxo-2,3- dihydro-1H- benzo[d]imidazol-5- yl)methyl)piperidin-1- yl)-2- oxoethoxy)pyrrolidin- 1-yl)-3,3-dimethyl-1- oxobutan-2- yl)carbamoyl)benzo[b] thiophen-5- yl)fluoromethyl) phosphonic acid or ((1R)-(2- (((2S)-1-((2S,3R)-2- ((6S,7R)-7-cyano-6- phenyl-4- azaspiro[2.4]heptane-4- carbonyl)-3-(2-(4-((1- (2,6-dioxopiperidin-3- yl)-3-methyl-2-oxo-2,3- dihydro-1H- benzo[d]imidazol-5- | 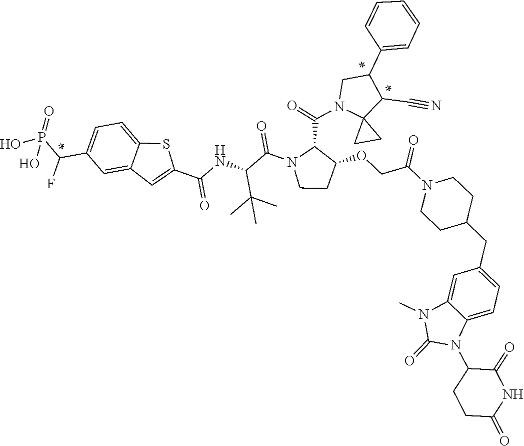 [SMILES: Cn1c(=O)n(C2CCC(=O)NC2=O)c2ccc(CC3CCN(C(=O)CO[C@@H]4CCN(C(=O)[C@@H](NC(=O)c5cc6cc(C(F)P(=O)(O)O)ccc6s5)C(C)(C)C)[C@@H]4C(=O)N4CC(c5ccccc5)C(C#N)C45CC5)CC3)cc21] |
| | yl)methyl)piperidin-1- | |
| | yl)-2- | |
| | oxoethoxy)pyrrolidin- | |
| | 1-yl)-3,3-dimethyl-1- | |
| | oxobutan-2- | |
| | yl)carbamoyl)benzo[b] | |
| | thiophen-5- | |
| | yl)fluoromethyl) | |
| | phosphonic acid | |
| | or ((1R)-(2- | |
| | (((2S)-1-((2S,3R)-2- | |
| | ((6R,7S)-7-cyano-6- | |
| | phenyl-4- | |
| | azaspiro[2.4]heptane-4- | |
| | carbonyl)-3-(2-(4-((1- | |
| | (2,6-dioxopiperidin-3- | |
| | yl)-3-methyl-2-oxo-2,3- | |
| | dihydro-1H- | |
| | benzo[d]imidazol-5- | |
| | yl)methyl)piperidin-1- | |
| | yl)-2- | |
| | oxoethoxy)pyrrolidin- | |
| | 1-yl)-3,3-dimethyl-1- | |
| | oxobutan-2- | |
| | yl)carbamoyl)benzo[b] | |
| | thiophen-5- | |
| | yl)fluoromethyl) | |
| | phosphonic acid | |
| | or ((1S)-(2- | |
| | (((2S)-1-((2S,3R)-2- | |
| | ((6R,7S)-7-cyano-6- | |
| | phenyl-4- | |
| | azaspiro[2.4]heptane-4- | |
| | carbonyl)-3-(2-(4-((1- | |
| | (2,6-dioxopiperidin-3- | |
| | yl)-3-methyl-2-oxo-2,3- | |
| | dihydro-1H- | |
| | benzo[d]imidazol-5- | |
| | yl)methyl)piperidin-1- | |
| | yl)-2- | |
| | oxoethoxy)pyrrolidin- | |
| | 1-yl)-3,3-dimethyl-1- | |
| | oxobutan-2- | |
| | yl)carbamoyl)benzo[b] | |
| | thiophen-5- | |
| | yl)fluoromethyl) | |
| | phosphonic acid | |
| 3.12 | ((2-(((2S)-1-((2S,3R)-3- ((1-(1-(2,6- dioxopiperidin-3-yl)-3- methyl-2-oxo-2,3- dihydro-1H- benzo[d]imidazol-5- yl)piperidin-4- yl)methyl)-2-((R)-2- phenylmorpholine-4- carbonyl)pyrrolidin-1- yl)-3,3-dimethyl-1- oxobutan-2- yl)carbamoyl)benzo[b] thiophen-5- yl)difluoromethyl) phosphonic acid | 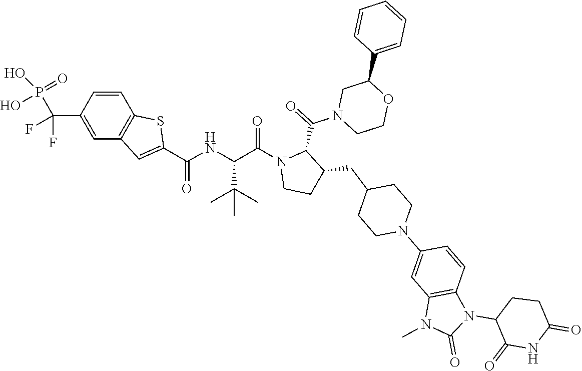 [SMILES: Cn1c(=O)n(C2CCC(=O)NC2=O)c2ccc(N3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21] |
| 3.13 | ((2-(((2S)-1-((2S,3R)-2- (4,4-difluoro-3- phenylpiperidine-1- carbonyl)-3-((1-((1- (2,6-dioxopiperidin-3- yl)-3-methyl-2-oxo-2,3- dihydro-1H- benzo[d]imidazol-5- yl)methyl)piperidin-4- yl)methyl)pyrrolidin-1- yl)-3,3-dimethyl-1- oxobutan-2- yl)carbamoyl)benzo[b] thiophen-5- yl)difluoromethyl) phosphonic acid | 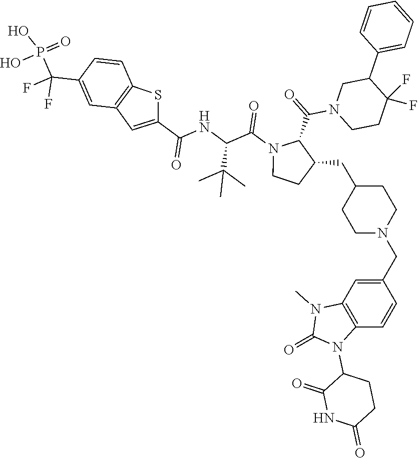 [SMILES: Cn1c(=O)n(C2CCC(=O)NC2=O)c2ccc(CN3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCC(F)(F)C(c5ccccc5)C4)CC3)cc21] |
| 3.14 | ((2-(((2S)-1-((2S,3R)-3- ((1-((1-(2,6- dioxopiperidin-3-yl)-3- methyl-2-oxo-2,3- dihydro-1H- benzo[d]imidazol-5- yl)methyl)piperidin-4- yl)methyl)-2-((R)-4- methyl-3- phenylpiperazine-1- carbonyl)pyrrolidin-1- yl)-3,3-dimethyl-1- oxobutan-2- yl)carbamoyl)benzo[b] thiophen-5- yl)difluoromethyl) phosphonic acid | 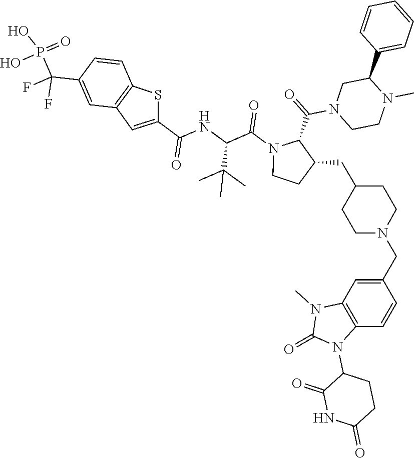 [SMILES: CN1CCN(C(=O)[C@@H]2[C@H](CC3CCN(Cc4ccc5c(c4)n(C)c(=O)n5C4CCC(=O)NC4=O)CC3)CCN2C(=O)[C@@H](NC(=O)c2cc3cc(C(F)(F)P(=O)(O)O)ccc3s2)C(C)(C)C)C[C@H]1c1ccccc1] |

TABLE 3.2
Procedures and analogs to Example 3.1 Synthesis.

| Example | Starting | Instead of | Variation from |
|---|---|---|---|
| Number | Material | B3.3 | Example 3.1 |
| 3.2 | C2.2 | B3.4 | |
| 3.3 | C5.3 | B3.3 | Step 1 performed in |
| | | | DCM overnight |
| 3.4 | C6.5 | B1.1 | |
| 3.5 | C7.1 | B1.1 | |
| 3.6 | C8.1 | B1.1 | |
| 3.7 | E2.6 | B1.1 | |
| 3.8 | E2.9 | B1.1 | |
| 3.9 | C5.4 | B1.1 | |
| 3.10 | E2.5 | B1.1 | |
| 3.11 | C7.2 | B3.3 | |
| 3.12 | C12.1 | B1.1 | |
| 3.13 | D2.2 | B1.1 | |
| 3.14 | D3.2 | B1.1 | |

TABLE 3.3
ES/MS and 1H NMR data for Examples 3.1 to 3.14

| Example | ES/MS | | |
|---|---|---|---|
| Number | m/z | Ion | 1H NMR |
| 3.1 | 1056.8 | [M − H]⁻ | (400 MHz, DMSO-d6) δ 11.08 (s, 1H), 8.55- |
| | | | 8.40 (m, 1H), 8.36-8.21 (m, 1H), 8.07- |
| | | | 7.85 (m, 2H), 7.57-7.49 (m, 1H), 7.46-7.08 |
| | | | (m, 5H), 7.04-6.92 (m, 2H), 6.87-6.74 (m, |
| | | | 1H), 5.93-5.75 (m, 1H), 5.44-1.40 (m, |
| | | | 35H), 1.25-0.89 (m, 9H). |
| 3.2 | 1056.7 | [M − H]⁻ | (400 MHz, DMSO-d6) δ 11.08 (s, 1H), 8.55- |
| | | | 8.39 (m, 1H), 8.35-8.21 (m, 1H), 8.07-8.00 |
| | | | (m, 1H), 8.00-7.86 (m, 1H), 7.58-7.47 (m, |
| | | | 1H), 7.47-7.12 (m, 5H), 7.06-6.93 (m, 2H), |
| | | | 6.88-6.75 (m, 1H), 5.94-5.73 (m, 1H), 5.53- |
| | | | 1.38 (m, 35H), 1.17-0.97 (m, 9H). |
| 3.3 | 1012.2 | [M − H]⁻ | (400 MHz, DMSO-d6) δ 11.16 (s, 1H), 8.64- |
| | | | 8.27 (m, 2H), 8.07 (d, J = 8.4 Hz, 1H), 8.05- |
| | | | 7.91 (m, 1H), 7.62-7.14 (m, 8H), 6.61-6.48 |
| | | | (m, 1H), 6.01-5.74 (m, 1H), 5.52-5.00 (m, |
| | | | 3H), 4.86-4.65 (m, 2H), 4.57-0.90 (m, |
| | | | 39H). |
| 3.4 | 1062.8 | [M + H]⁺ | (400 MHz, Methanol-d4) δ 8.29-8.15 (m, |
| | | | 2H), 8.10-7.98 (m, 2H), 7.83 (d, J = 1.4 Hz, |
| | | | 1H), 7.70 (d, J = 8.7 Hz, 1H), 7.50-7.21 (m, |
| | | | 6H), 7.05-6.78 (m, 3H), 5.31 (dd, J = 11.8, |
| | | | 5.8 Hz, 2H), 5.15 (d, J = 6.2 Hz, 1H), 4.91 (d, |
| | | | J = 3.7 Hz, 1H), 4.60-4.20 (m, 7H), 3.80- |
| | | | 3.40 (m, 4H), 2.92-2.13 (m, 11H), 1.88- |
| | | | 1.02 (m, 11H). |
| 3.5 | 1029.9 | [M − H]⁻ | (500 MHz, DMSO-d6) δ 10.79 (s, 1H), 8.50- |
| | | | 8.40 (m, 1H), 8.17-7.93 (m, 3H), 7.69-7.63 |
| | | | (m, 1H), 7.51-7.28 (m, 5H), 7.05-6.93 (m, |
| | | | 2H), 6.90-6.81 (m, 1H), 5.52-0.77 (m, |
| | | | 44H). |
| 3.6 | 1034.0 | [M + H]⁺ | (400 MHz, DMSO) δ 11.12 (s, 1H), 9.55- |
| | | | 0.60 (m, 55H). |
| 3.7 | 1016.1 | [M − H]⁻ | (400 MHz, DMSO-d6) δ 11.16 (s, 1H), 8.62- |
| | | | 8.04 (m, 5H), 7.67-7.13 (m, 8H), 5.50-0.97 |
| | | | (m, 42H). |
| 3.8 | 988.1 | [M − H]⁻ | (400 MHz, DMSO-d6) δ 11.13 (s, 1H), 8.60- |
| | | | 8.01 (m, 5H), 7.64-7.07 (m, 8H), 5.60-0.50 |
| | | | (m, 38H). |
| 3.9 | 1044.0 | [M − H]⁻ | (400 MHz, DMSO-d6) δ 11.13 (s, 1H), 9.13 |
| | | | (s, 1H), 8.50-8.09 (m, 2H), 8.05 (s, 1H), 7.60 |
| | | | (d, J = 8.6 Hz, 1H), 7.51-7.06 (m, 9H), 5.42 |
| | | | (dd, J = 12.8, 5.5 Hz, 1H), 5.28-4.86 |
| | | | (m, 1H), 4.85-4.60 (m, 2H), 4.51-3.71 (m, |
| | | | 6H), 3.66-3.44 (m, 17H), 2.99-2.60 (m, |
| | | | 4H), 2.41-1.16 (m, 6H), 1.14-0.82 (m, 9H). |
| 3.10 | 1016.1 | [M − H]⁻ | 1H NMR (400 MHz, DMSO-d6) δ 11.16 (s, |
| | | | 1H), 8.66-8.31 (m, 2H), 8.22-8.03 (m, 2H), |
| | | | 7.63 (d, J = 8.6 Hz, 1H), 7.53-7.14 (m, 8H), |
| | | | 5.57-0.78 (m, 42H). |
| 3.11 | 1093.4 | [M + H]⁺ | (400 MHz, DMSO-d6) δ 11.09 (s, 1H), 8.48 |
| | | | (s, 1H), 8.29 (d, J = 8.7 Hz, 1H), 8.14-7.88 |
| | | | (m, 2H), 7.64-7.50 (m, 3H), 7.50-7.19 (m, |
| | | | 6H), 7.02 (d, J = 8.9 Hz, 2H), 6.98-6.65 (m, |
| | | | 1H), 6.06-5.75 (m, 1H), 5.35 (dd, J = 12.7, |
| | | | 5.4 Hz, 1H), 5.19-4.96 (m, 1H), 4.84 (d, J = |
| | | | 7.2 Hz, 1H), 4.77-4.55 (m, 1H), 4.55-3.89 |
| | | | (m, 6H), 3.89-3.66 (m, 4H), 3.57 (s, 1H), |
| | | | 2.90 (m, 2H), 2.77-2.59 (m, 2H), 2.25-1.90 |
| | | | (m, 6H), 1.76 (s, 1H), 1.58 (d, J = 12.7 Hz, |
| | | | 2H), 1.31 (m, 2H), 1.08 (s, 9H), 1.01-0.49 |
| | | | (m, 4H). |
| 3.12 | 1016.1 | [M − H]⁻ | 1H NMR (400 MHz, DMSO-d6) δ 11.14 (s, |
| | | | 1H), 8.64-8.27 (m, 3H), 8.25-7.97 (m, 2H), |
| | | | 7.63 (d, J = 8.6 Hz, 1H), 7.59-7.01 (m, 7H), |
| | | | 5.63-0.72 (m, 42H). |
| 3.13 | 1066.0 | [M + H]⁺ | 1H NMR (500 MHz, DMSO-d6) δ 10.84 (s, |
| | | | 1H), 8.51-8.35 (m, 1H), 8.19-8.00 (m, 3H), |
| | | | 7.69-7.60 (m, 1H), 7.45-7.24 (m, 6H), 7.23- |
| | | | 7.11 (m, 2H), 5.45-0.84 (m, 44H). |
| 3.14 | 1043.2 | [M − H]⁻ | 1H NMR (400 MHz, DMSO) δ 11.13 (s, 1H), |
| | | | 9.53-9.11 (m, 1H), 8.47-8.30 (m, 2H), 8.20- |
| | | | 8.01 (m, 2H), 7.62-7.13 (m, 8H), 9.97- |
| | | | 0.63 (m, 55H). |

### Example 4.1: ((2-(((2S)-1-((2S,3R)-3-((7-(1-(2,6-dioxopiperidin-3-yl)-3-methyl-2-oxo-2,3-dihydro-1H-benzo[d]imidazol-5-yl)hept-6-yn-1-yl)oxy)-2-((R)-2-phenylmorpholine-4-carbonyl)pyrrolidin-1-yl)-3,3-dimethyl-1-oxobutan-2-yl)carbamoyl)benzo[b]thiophen-5-yl)difluoromethyl)phosphonic acid

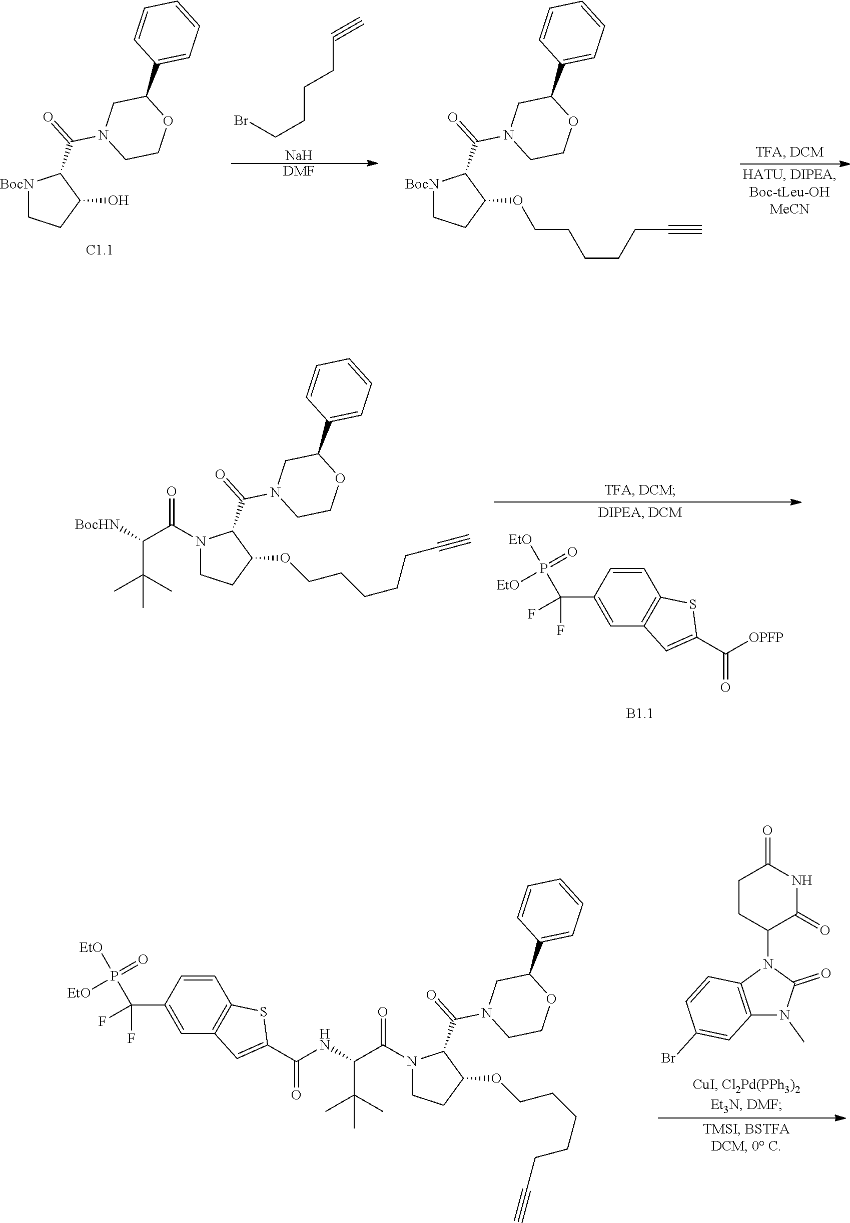

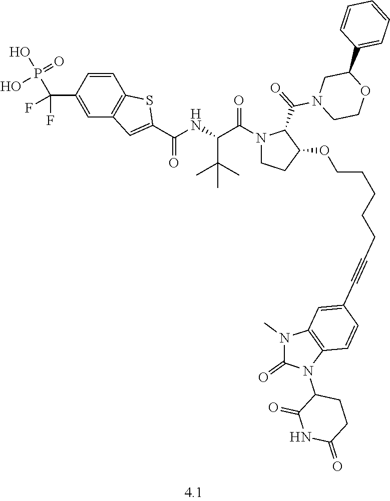 [SMILES: Cn1c(=O)n(C2CCC(=O)NC2=O)c2ccc(C#CCCCCCO[C@@H]3CCN(C(=O)[C@@H](NC(=O)c4cc5cc(C(F)(F)P(=O)(O)O)ccc5s4)C(C)(C)C)[C@@H]3C(=O)N3CCO[C@H](c4ccccc4)C3)cc21]

tert-butyl (2S,3R)-3-(hept-6-yn-1-yloxy)-2-((R)-2-phenylmorpholine-4-carbonyl)pyrrolidine-1-carboxylate (C1.1). NaH (60% in mineral oil, 12 mg, 0.3 mmol) was added to a solution of tert-butyl (2S,3R)-3-hydroxy-2-((R)-2-phenylmorpholine-4-carbonyl)pyrrolidine-1-carboxylate (75 mg, 0.2 mmol) in DMF (2 mL). After stirring for 30 minutes, 7-bromohept-1-yne (38 mg, 0.22 mmol) was added. The mixture was allowed to stir overnight before being quenched by sat. aq. NH₄Cl and diluted with EtOAc. The organic layer was separated, dried over MgSO₄, filtered, and concentrated to afford crude tert-butyl (2S,3R)-3-(hept-6-yn-1-yloxy)-2-((R)-2-phenylmorpholine-4-carbonyl)pyrrolidine-1-carboxylate which was carried forward without further purification. ES/MS: m/z=471.0 [M+H]⁺.

tert-butyl ((S)-1-((2S,3R)-3-(hept-6-yn-1-yloxy)-2-((R)-2-phenylmorpholine-4-carbonyl)pyrrolidin-1-yl)-3,3-dimethyl-1-oxobutan-2-yl)carbamate. The crude tert-butyl (2S,3R)-3-(hept-6-yn-1-yloxy)-2-((R)-2-phenylmorpholine-4-carbonyl)pyrrolidine-1-carboxylate from the previous step was suspended in HCl (4.0M in dioxane, 0.5 mL, 2.0 mmol). After stirring for 3 hours, the mixture was concentrated and resuspended in DMF (2.0 mL). (2S)-2-(tert-butoxycarbonylamino)-3,3-dimethyl-butanoic acid (51 mg, 0.22 mmol), DIPEA (0.14 mL, 0.8 mmol), and HATU (113 mg, 0.3 mmol) were then added. After stirring for 1 hour, the mixture was diluted with EtOAc and washed with 10% aq. LiCl. The organic layer was dried over MgSO₄, filtered, and concentrated. Purification by silica gel flash column chromatography afforded tert-butyl ((S)-1-((2S,3R)-3-(hept-6-yn-1-yloxy)-2-((R)-2-phenylmorpholine-4-carbonyl)pyrrolidin-1-yl)-3,3-dimethyl-1-oxobutan-2-yl)carbamate. ES/MS: m/z=584.2 [M+H]⁺.

diethyl (difluoro(2-(((S)-1-((2S,3R)-3-(hept-6-yn-1-yloxy)-2-((R)-2-phenylmorpholine-4-carbonyl)pyrrolidin-1-yl)-3,3-dimethyl-1-oxobutan-2-yl)carbamoyl)benzo[b]thiophen-5-yl)methyl)phosphonate. tert-butyl ((S)-1-((2S,3R)-3-(hept-6-yn-1-yloxy)-2-((R)-2-phenylmorpholine-4-carbonyl)pyrrolidin-1-yl)-3,3-dimethyl-1-oxobutan-2-yl)carbamate (47 mg, 0.08 mmol) was suspended in DCM/TFA (4:1, 2.0 mL). After stirring for 1 hour, the mixture was concentrated and resuspended in DCM (2 mL). (2,3,4,5,6-pentafluorophenyl) 5-[diethoxyphosphoryl(difluoro)methyl]benzothiophene-2-carboxylate (B1.1, 47 mg, 0.09 mmol) and DIPEA (0.14 mL, 0.81 mmol) were added. After stirring overnight, the mixture was concentrated and purified by silica gel flash column chromatography to afford diethyl (difluoro(2-(((S)-1-((2S,3R)-3-(hept-6-yn-1-yloxy)-2-((R)-2-phenylmorpholine-4-carbonyl)pyrrolidin-1-yl)-3,3-dimethyl-1-oxobutan-2-yl)carbamoyl)benzo[b]thiophen-5-yl)methyl)phosphonate. ES/MS: m/z=827.8 [M−H]⁻.

((2-(((2S)-1-((2S,3R)-3-((7-(1-(2,6-dioxopiperidin-3-yl)-3-methyl-2-oxo-2,3-dihydro-1H-benzo[d]imidazol-5-yl)hept-6-yn-1-yl)oxy)-2-((R)-2-phenylmorpholine-4-carbonyl)pyrrolidin-1-yl)-3,3-dimethyl-1-oxobutan-2-yl)carbamoyl)benzo[b]thiophen-5-yl)difluoromethyl)phosphonic acid (4.1). A mixture of diethyl (difluoro(2-(((S)-1-((2S,3R)-3-(hept-6-yn-1-yloxy)-2-((R)-2-phenylmorpholine-4-carbonyl)pyrrolidin-1-yl)-3,3-dimethyl-1-oxobutan-2-yl)carbamoyl)benzo[b]thiophen-5-yl)methyl)phosphonate (66 mg, 0.08 mmol), 3-(5-bromo-3-methyl-2-oxo-2,3-dihydro-1H-benzo[d]imidazol-1-yl)piperidine-2,6-dione (27 mg, 0.08 mmol), CuI (3.0 mg, 0.02 mmol), and Cl₂Pd(PPh₃)₂ (6.0 mg, 0.008 mmol) in DMF/Et₃N (1:1, 1 mL) was heated to 100° C. for 3 hours. The mixture was cooled to room temperature, diluted with EtOAc, and filtered over Celite. The filtrate was concentrated, resuspended in DCM (2 mL), and cooled to 0° C. N,O-Bis(trimethylsilyl)trifluoroacetamide (0.19 mL, 0.72 mmol) and TMSI (1.0M in DCM, 0.24 mL, 0.24 mmol) were added at 0° C. After 30 minutes, the mixture was concentrated. Purification by reverse phase HPLC afforded ((2-(((2S)-1-((2S,3R)-3-((7-(1-(2,6-dioxopiperidin-3-yl)-3-methyl-2-oxo-2,3-dihydro-1H-benzo[d]imidazol-5-yl)hept-6-yn-1-yl)oxy)-2-((R)-2-phenylmorpholine-4-carbonyl)pyrrolidin-1-yl)-3,3-dimethyl-1-oxobutan-2-yl)carbamoyl)benzo[b]thiophen-5-yl)difluoromethyl)phosphonic acid.

TABLE 4
ES/MS and 1H NMR data for Example 4.1

| Example | ES/MS | | |
|---|---|---|---|
| Number | m/z | Ion | 1H NMR |
| 4.1 | 1028.7 | [M − H]⁻ | (400 MHz, DMSO-d6) δ 11.13 (s, 1H), 8.59- |
| | | | 8.50 (m, 1H), 8.39-8.24 (m, 1H), 8.19-8.10 |
| | | | (m, 1H), 8.06 (s, 1H), 7.64-7.53 (m, 1H), |
| | | | 7.42-6.91 (m, 7H), 6.52-6.39 (m, 1H), 5.43- |
| | | | 5.00 (m, 3H), 4.82-0.93 (m, 38H). |

### Example 5.1: Propyl (((2-(((2S)-1-((2S,3R)-3-(2-(4-((1-(2,6-dioxopiperidin-3-yl)-3-methyl-2-oxo-2,3-dihydro-1H-benzo[d]imidazol-5-yl)methyl)piperidin-1-yl)-2-oxoethoxy)-2-((R)-2-phenylmorpholine-4-carbonyl)pyrrolidin-1-yl)-3,3-dimethyl-1-oxobutan-2-yl)carbamoyl)benzo[b]thiophen-5-yl)fluoromethyl)(phenoxy)phosphoryl)-L-alaninate

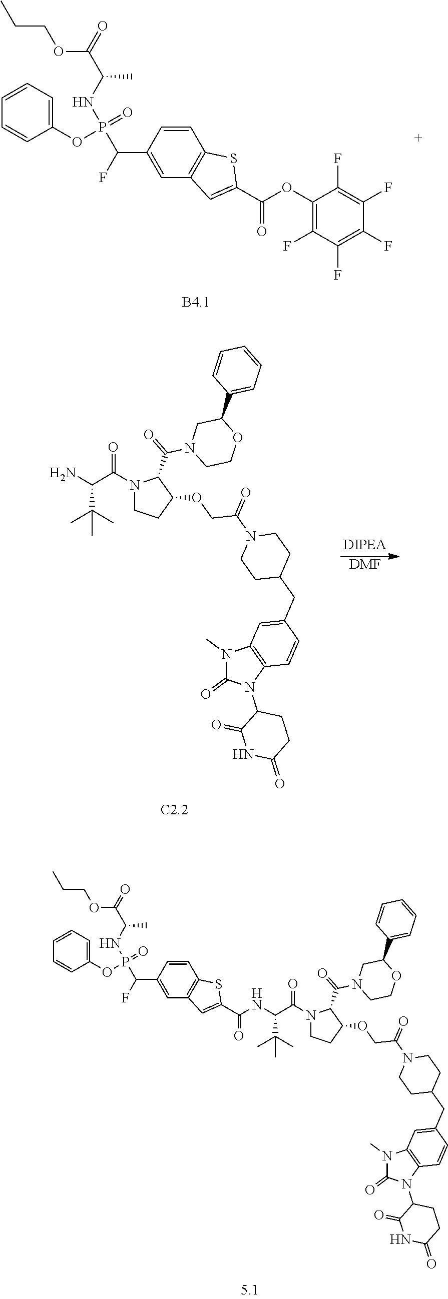

A round bottom flask was charged with perfluorophenyl 5-(fluoro((((S)-1-oxo-1-propoxypropan-2-yl)amino)(phenoxy)phosphoryl)methyl)benzo[b]thiophene-2-carboxylate B4.1 (30.0 mg, 0.0465 mmol, 1.0 equiv.) and 3-(5-((1-(2-(((2S,3R)-1-((S)-2-amino-3,3-dimethylbutanoyl)-2-((R)-2-phenylmorpholine-4-carbonyl)pyrrolidin-3-yl)oxy)acetyl)piperidin-4-yl)methyl)-3-methyl-2-oxo-2,3-dihydro-1H-benzo[d]imidazol-1-yl)piperidine-2,6-dione trifluoroacetate (41.8 mg, 0.0465 mmol, 1.0 equiv.). The reagents were dissolved in DMF (3.0 mL) and DIPEA (0.08 mL, 0.465 mmol, 10.0 equiv.) was added at rt. The solution was stirred at rt for 23 min, prior to quenching with water and purifying by neutral prep-HPLC (10-90% ACN in water) to afford 5.1.

The compounds in Table 5.1 were prepared analogously to Example 5.1 modified as described in Table 5.2

TABLE 5.1
Examples 5.2 to 5.16

| Ex. | Name | Structure |
|---|---|---|
| 5.2 | propyl ((S)-((1R)-(2-(((2S)-1- ((2S,3R)-3-(2-(4-((1-(2,6- dioxopiperidin-3-yl)-3-methyl- 2-oxo-2,3-dihydro-1H- benzo[d]imidazol-5- yl)methyl)piperidin-1-yl)-2- oxoethoxy)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or propyl ((S)-((1S)-(2-(((2S)-1- ((2S,3R)-3-(2-(4-((1-(2,6- dioxopiperidin-3-yl)-3-methyl- 2-oxo-2,3-dihydro-1H- benzo[d]imidazol-5- yl)methyl)piperidin-1-yl)-2- oxoethoxy)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or propyl ((R)-((1S)-(2-(((2S)-1- ((2S,3R)-3-(2-(4-((1-(2,6- dioxopiperidin-3-yl)-3-methyl- 2-oxo-2,3-dihydro-1H- benzo[d]imidazol-5- yl)methyl)piperidin-1-yl)-2- oxoethoxy)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or propyl ((R)-((1R)-(2-(((2S)-1- ((2S,3R)-3-(2-(4-((1-(2,6- dioxopiperidin-3-yl)-3-methyl- 2-oxo-2,3-dihydro-1H- benzo[d]imidazol-5- yl)methyl)piperidin-1-yl)-2- oxoethoxy)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate | 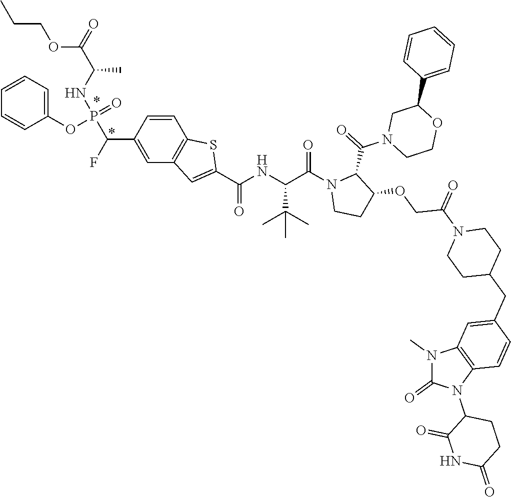 [SMILES: CCCOC(=O)[C@H](C)NP(=O)(Oc1ccccc1)C(F)c1ccc2sc(C(=O)N[C@H](C(=O)N3CC[C@@H](OCC(=O)N4CCC(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1] |
| 5.3 | propyl ((S)-((1R)-(2-(((2S)-1- ((2S,3R)-3-(2-(4-((1-(2,6- dioxopiperidin-3-yl)-3-methyl- 2-oxo-2,3-dihydro-1H- benzo[d]imidazol-5- yl)methyl)piperidin-1-yl)-2- oxoethoxy)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or propyl ((S)-((1S)-(2-(((2S)-1- ((2S,3R)-3-(2-(4-((1-(2,6- dioxopiperidin-3-yl)-3-methyl- 2-oxo-2,3-dihydro-1H- benzo[d]imidazol-5- yl)methyl)piperidin-1-yl)-2- oxoethoxy)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or propyl ((R)-((1S)-(2-(((2S)-1- ((2S,3R)-3-(2-(4-((1-(2,6- dioxopiperidin-3-yl)-3-methyl- 2-oxo-2,3-dihydro-1H- benzo[d]imidazol-5- yl)methyl)piperidin-1-yl)-2- oxoethoxy)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or propyl ((R)-((1R)-(2-(((2S)-1- ((2S,3R)-3-(2-(4-((1-(2,6- dioxopiperidin-3-yl)-3-methyl- 2-oxo-2,3-dihydro-1H- benzo[d]imidazol-5- yl)methyl)piperidin-1-yl)-2- oxoethoxy)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate | 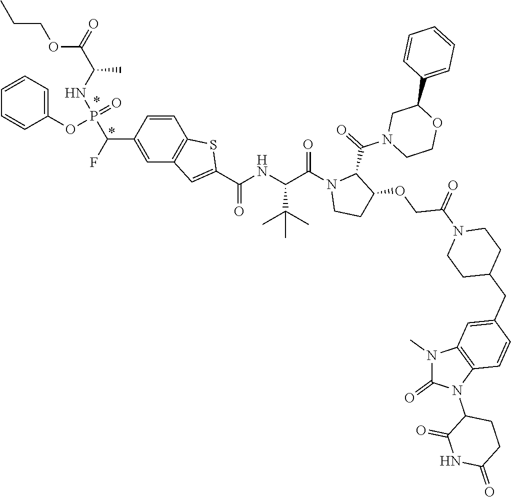 [SMILES: CCCOC(=O)[C@H](C)NP(=O)(Oc1ccccc1)C(F)c1ccc2sc(C(=O)N[C@H](C(=O)N3CC[C@@H](OCC(=O)N4CCC(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1] |
| 5.4 | propyl ((R)-((1R)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or propyl ((R)-((1S)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or propyl ((S)-((1S)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or propyl ((S)-((1R)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate | 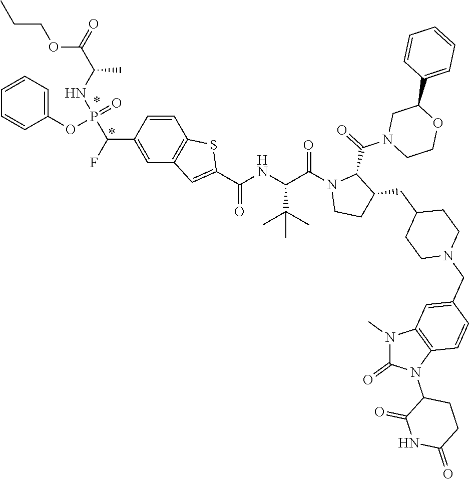 [SMILES: CCCOC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1] |
| 5.5 | propyl ((R)-((1R)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or propyl ((R)-((1S)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or propyl ((S)-((1S)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or propyl ((S)-((1R)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate | 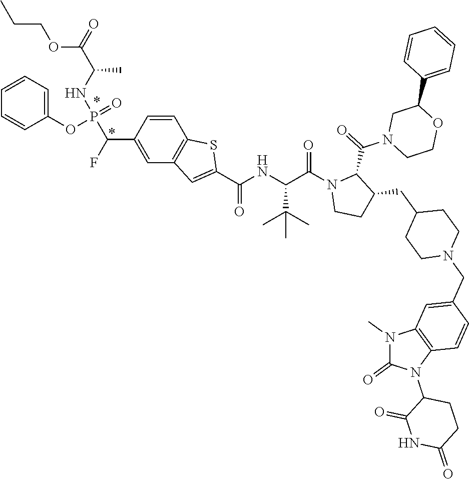 [SMILES: CCCOC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1] |
| 5.6 | dipropyl 2,2′-((((2-(((2S)-1- ((2S,3R)-3-(2-(4-((1-(2,6- dioxopiperidin-3-yl)-3-methyl- 2-oxo-2,3-dihydro-1H- benzo[d]imidazol-5- yl)methyl)piperidin-1-yl)-2- oxoethoxy)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)phosphoryl) bis(azanediyl))(2S,2′S)- dipropionate | 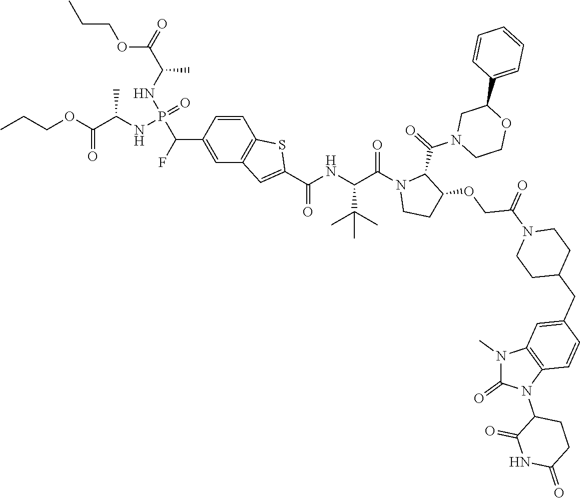 [SMILES: CCCOC(=O)[C@H](C)NP(=O)(N[C@@H](C)C(=O)OCCC)C(F)c1ccc2sc(C(=O)N[C@H](C(=O)N3CC[C@@H](OCC(=O)N4CCC(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1] |
| 5.7 | ethyl (((1R)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or ethyl (((1S)-(2-(((2S)-1-((2S,3R)-3- ((1-((1-(2,6-dioxopiperidin-3- yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or ethyl (((1S)-(2-(((2S)-1-((2S,3R)-3- ((1-((1-(2,6-dioxopiperidin-3- yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or ethyl (((1R)-(2-(((2S)-1-((2S,3R)-3- ((1-((1-(2,6-dioxopiperidin-3- yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate | 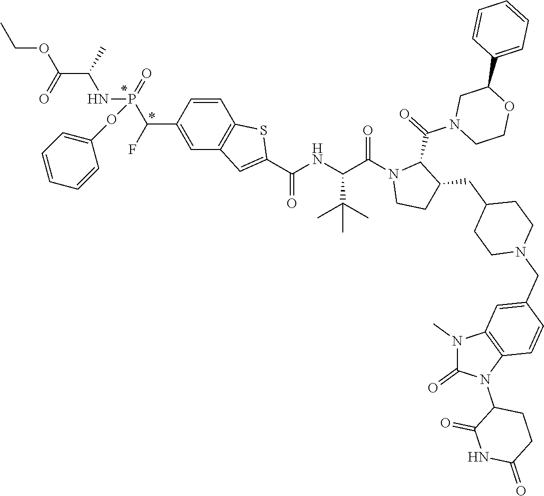 [SMILES: CCOC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1] |
| 5.8 | methyl (((1R)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or methyl (((1S)-(2-(((2S)-1-((2S,3R)-3- ((1-((1-(2,6-dioxopiperidin-3- yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or methyl (((1S)-(2-(((2S)-1-((2S,3R)-3- ((1-((1-(2,6-dioxopiperidin-3- yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or methyl (((1R)-(2-(((2S)-1-((2S,3R)-3- ((1-((1-(2,6-dioxopiperidin-3- yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate | 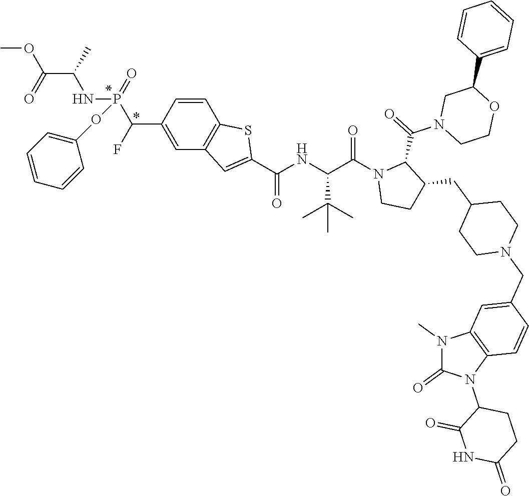 [SMILES: COC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1] |
| 5.9 | methyl (((1R)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or methyl (((1S)-(2-(((2S)-1-((2S,3R)-3- ((1-((1-(2,6-dioxopiperidin-3- yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or methyl (((1S)-(2-(((2S)-1-((2S,3R)-3- ((1-((1-(2,6-dioxopiperidin-3- yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or methyl (((1R)-(2-(((2S)-1-((2S,3R)-3- ((1-((1-(2,6-dioxopiperidin-3- yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate | 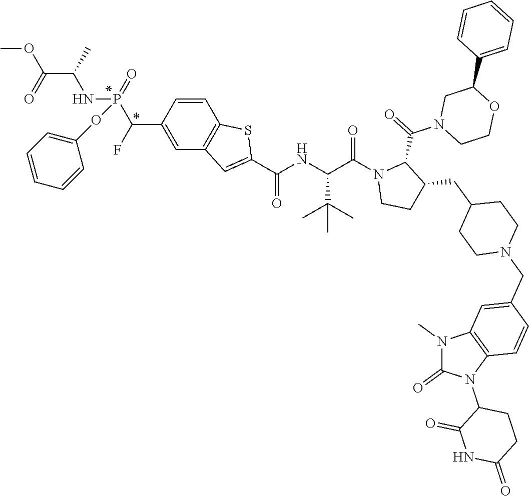 [SMILES: COC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1] |
| 5.10 | dipropyl 2,2′-((((1S)-(2-(((2S)- 1-((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)-4- fluorobenzo[b]thiophen-5- yl)fluoromethyl)phosphoryl) bis(azanediyl))(2S,2′S)- dipropionate or dipropyl 2,2′- ((((1R)-(2-(((2S)-1-((2S,3R)- 3-((1-((1-(2,6-dioxopiperidin- 3-yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)-4- fluorobenzo[b]thiophen-5- yl)fluoromethyl)phosphoryl) bis(azanediyl))(2S,2′S)- dipropionate | 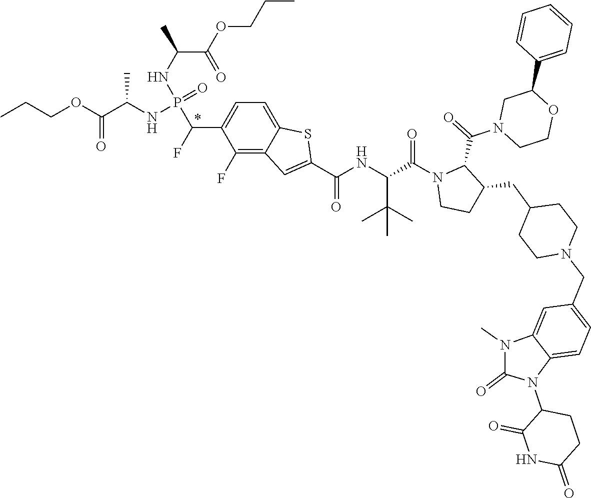 [SMILES: CCCOC(=O)[C@H](C)NP(=O)(N[C@@H](C)C(=O)OCCC)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1F] |
| 5.11 | ethyl (((1R)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or ethyl (((1S)-(2-(((2S)-1-((2S,3R)-3- ((1-((1-(2,6-dioxopiperidin-3- yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or ethyl (((1S)-(2-(((2S)-1-((2S,3R)-3- ((1-((1-(2,6-dioxopiperidin-3- yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L- alaninate or ethyl (((1R)-(2-(((2S)-1-((2S,3R)-3- ((1-((1-(2,6-dioxopiperidin-3- yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate | 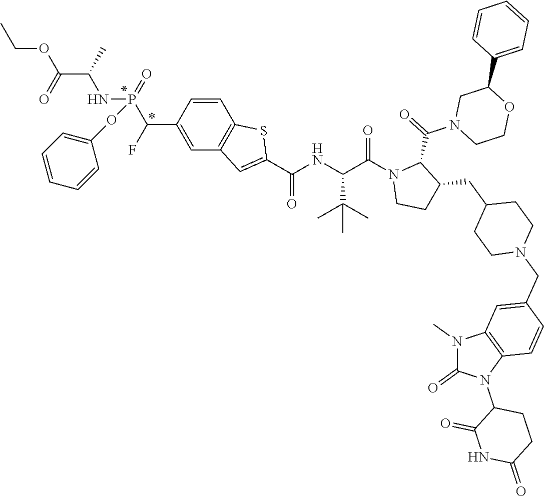 [SMILES: CCOC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1] |
| 5.12 | isopropyl (((1R)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate or isopropyl (((1S)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate or isopropyl (((1S)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate or isopropyl (((1R)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate | 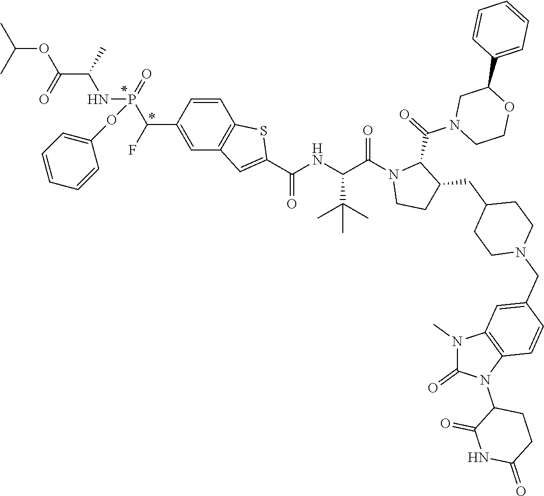 [SMILES: CC(C)OC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1] |
| 5.13 | isopropyl (((1R)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate or isopropyl (((1S)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate or isopropyl (((1S)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate or isopropyl (((1R)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5- yl)fluoromethyl)(phenoxy) phosphoryl)-L-alaninate | 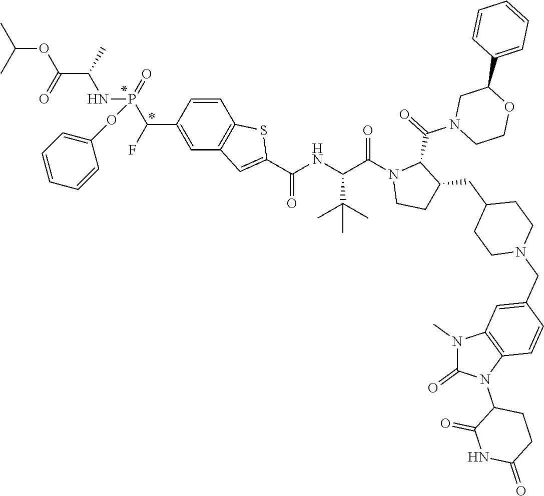 [SMILES: CC(C)OC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1] |
| 5.14 | propyl (((1R)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)-4- fluorobenzo[b]thiophen-5- yl)fluoromethyl)(phenoxy) phosphoryl)-L- alaninate or propyl (((1S)-(2-(((2S)-1-((2S,3R)-3- ((1-((1-(2,6-dioxopiperidin-3- yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)-4- fluorobenzo[b]thiophen-5- yl)fluoromethyl)(phenoxy) phosphoryl)-L-alaninate | 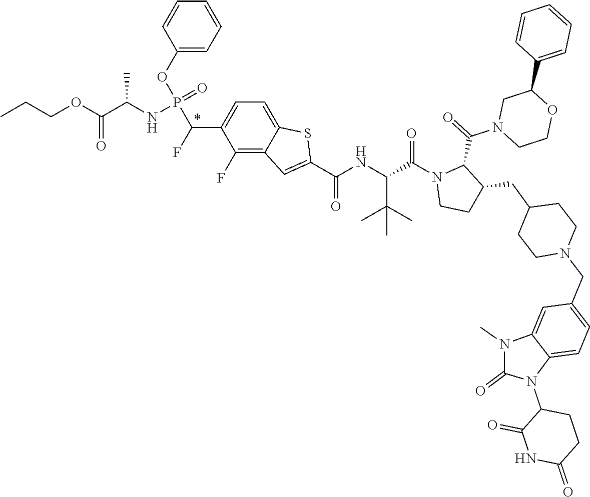 [SMILES: CCCOC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1F] |
| 5.15 | (R)-P-((1R)-(2-(((2S)-1- ((2S,3R)-3-(2-(4-((1-(2,6- dioxopiperidin-3-yl)-3-methyl- 2-oxo-2,3-dihydro-1H- benzo[d]imidazol-5- yl)methyl)piperidin-1-yl)-2- oxoethoxy)-2-((R)-2- phenylmorpholine-4- carbonyl)pyrrolidin-1-yl)-3,3- dimethyl-1-oxobutan-2- yl)carbamoyl)benzo[b] thiophen-5-yl)fluoromethyl)- N-((S)-1- oxo-1-propoxypropan-2- yl)phosphonamidic acid or (R)- P-((1S)-(2-(((2S)-1-((2S,3R)- 3-(2-(4-((1-(2,6- dioxopiperidin-3-yl)-3-methyl- 2-oxo-2,3-dihydro-1H- benzo[d]imidazol-5- yl)methyl)piperidin-1-yl)-2- oxoethoxy)-2-((R)-2- phenylmorpholine-4- carbonyl)pyrrolidin-1-yl)-3,3- dimethyl-1-oxobutan-2- yl)carbamoyl)benzo[b] thiophen-5-yl)fluoro- methyl)-N-((S)-1- oxo-1-propoxypropan-2- yl)phosphonamidic acid or (S)- P-((1S)-(2-(((2S)-1-((2S,3R)- 3-(2-(4-((1-(2,6- dioxopiperidin-3-yl)-3-methyl- 2-oxo-2,3-dihydro-1H- benzo[d]imidazol-5- yl)methyl)piperidin-1-yl)-2- oxoethoxy)-2-((R)-2- phenylmorpholine-4- carbonyl)pyrrolidin-1-yl)-3,3- dimethyl-1-oxobutan-2- yl)carbamoyl)benzo[b] thiophen-5-yl)fluoro- methyl)-N-((S)-1- oxo-1-propoxypropan-2- yl)phosphonamidic acid or (S)- P-((1R)-(2-(((2S)-1-((2S,3R)- 3-(2-(4-((1-(2,6- dioxopiperidin-3-yl)-3-methyl- 2-oxo-2,3-dihydro-1H- benzo[d]imidazol-5- yl)methyl)piperidin-1-yl)-2- oxoethoxy)-2-((R)-2- phenylmorpholine-4- carbonyl)pyrrolidin-1-yl)-3,3- dimethyl-1-oxobutan-2- yl)carbamoyl)benzo[b] thiophen-5-yl)fluoro- methyl)-N-((S)-1- oxo-1-propoxypropan-2- yl)phosphonamidic acid | 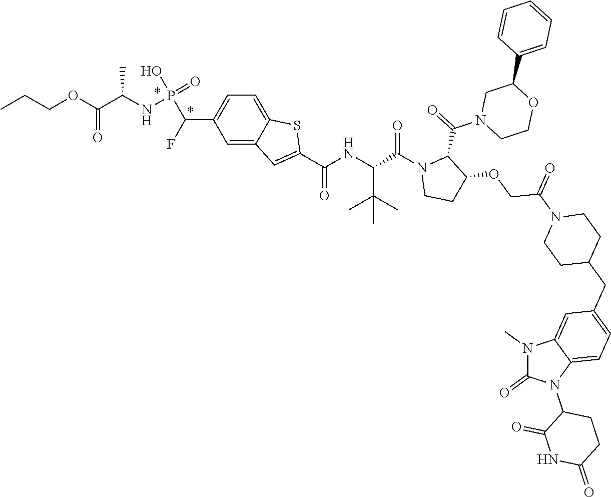 [SMILES: CCCOC(=O)[C@H](C)NP(=O)(O)C(F)c1ccc2sc(C(=O)N[C@H](C(=O)N3CC[C@@H](OCC(=O)N4CCC(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1] |
| 5.16 | (S)-P-((1R)-(2-(((2S)-1- ((2S,3R)-3-((1-((1-(2,6- dioxopiperidin-3-yl)-3-methyl- 2-oxo-2,3-dihydro-1H- benzo[d]imidazol-5- yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5-yl)fluoro- methyl)-N-((S)-1- oxo-1-propoxypropan-2- yl)phosphonamidic acid or (S)- P-((1S)-(2-(((2S)-1-((2S,3R)- 3-((1-((1-(2,6-dioxopiperidin- 3-yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5-yl)fluoro- methyl)-N-((S)-1- oxo-1-propoxypropan-2- yl)phosphonamidic acid or (R)- P-((1S)-(2-(((2S)-1-((2S,3R)- 3-((1-((1-(2,6-dioxopiperidin- 3-yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5-yl)fluoro- methyl)-N-((S)-1- oxo-1-propoxypropan-2- yl)phosphonamidic acid or (R)- P-((1R)-(2-(((2S)-1-((2S,3R)- 3-((1-((1-(2,6-dioxopiperidin- 3-yl)-3-methyl-2-oxo-2,3- dihydro-1H-benzo[d]imidazol- 5-yl)methyl)piperidin-4- yl)methyl)-2-((R)-2- phenylmorpholine-4- carbonyl)pyrrolidin-1-yl)-3,3- dimethyl-1-oxobutan-2- yl)carbamoyl)benzo[b] thiophen-5-yl)fluoro- methyl)-N-((S)-1- oxo-1-propoxypropan-2- yl)phosphonamidic acid | 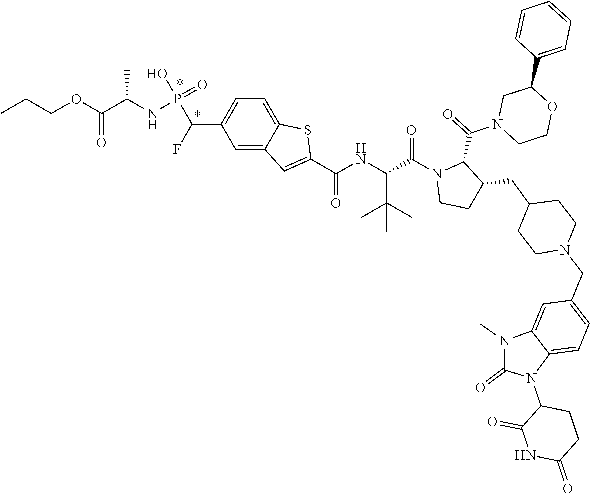 [SMILES: CCCOC(=O)[C@H](C)NP(=O)(O)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1] |

TABLE 5.2
Procedures and analogs to Example 5.1 Synthesis.

| Example | Starting | Instead of | Variation from |
|---|---|---|---|
| Number | Material | B4.1 | Example 5.1 |
| 5.2 | C2.2 | B5.7 | |
| 5.3 | C2.2 | B5.8 | |
| 5.4 | C5.3 | B5.7 | 15 equivalents of DIPEA used |
| | | | for 2 hours |
| 5.5 | C5.3 | B5.8 | 15 equivalents of DIPEA used |
| | | | for 2 hours |
| 5.6 | C2.2 | B6.4 | |
| 5.7 | C5.3 | B11.2 | |
| 5.8 | C5.3 | B13.1 | |
| 5.9 | C5.3 | B13.2 | |
| 5.10 | C5.3 | B9.1 | Reaction run in ACN with 1.2 |
| | | | equivalents of HATU |
| 5.11 | C5.3 | B11.1 | |
| 5.12 | C5.3 | B12.1 | |
| 5.13 | C5.3 | B12.2 | |
| 5.14 | C5.3 | B10.3 | Reaction run in ACN with 1.2 |
| | | | equivalents of HATU |
| 5.15 | C2.2 | B5.8 | 5.15 was a byproduct obtained |
| | | | in the reaction to access 5.3 |
| 5.16 | C5.3 | B5.8 | 5.16 was a byproduct obtained |
| | | | in the reaction to access 5.5 |

TABLE 5.3
ES/MS and 1H NMR data for Examples 5.1 to 5.16

| Example | ES/MS | | |
|---|---|---|---|
| Number | m/z | Ion | 1H NMR |
| 5.1 | 1245.7 | [M − H]⁻ | (400 MHz, DMSO-d6) δ 11.08 (s, 1H), 8.56- |
| | | | 8.27 (m, 2H), 8.13-7.90 (m, 2H), 7.66- |
| | | | 7.55 (m, 1H), 7.49-7.07 (m, 10H), 7.05- |
| | | | 6.90 (m, 2H), 6.89-6.73 (m, 1H), 6.33-6.03 |
| | | | (m, 2H), 5.48-5.00 (m, 2H), 4.83-4.67 (m, |
| | | | 1H), 4.61-0.67 (m, 52H). |
| 5.2 | 1246.2 | [M − H]⁻ | (400 MHz, DMSO-d6) δ 11.08 (s, 1H), 8.58- |
| | | | 8.27 (m, 2H), 8.12-7.99 (m, 2H), 7.62 (d, J = |
| | | | 8.5 Hz, 1H), 7.48-7.07 (m, 10H), 7.05- |
| | | | 6.92 (m, 2H), 6.87-6.74 (m, 1H), 6.28-6.01 |
| | | | (m, 2H), 5.48-5.02 (m, 2H), 4.81-4.67 (m, |
| | | | 1H), 4.62-1.29 (m, 37H), 1.26-0.89 (m, |
| | | | 12H), 0.81-0.69 (m, 3H). |
| 5.3 | 1246.2 | [M − H]⁻ | (400 MHz, DMSO-d6) δ 11.08 (s, 1H), 8.63- |
| | | | 8.26 (m, 2H), 8.14-7.95 (m, 2H), 7.61 (d, |
| | | | J = 8.5 Hz, 1H), 7.48-6.91 (m, 12H), 6.89- |
| | | | 6.74 (m, 1H), 6.31-6.06 (m, 2H), 5.46-5.00 |
| | | | (m, 2H), 4.85-4.65 (m, 1H), 4.62-1.39 (m, |
| | | | 37H), 1.27-0.90 (m, 12H), 0.85-0.77 (m, |
| | | | 3H). |
| 5.4 | 1201.2 | [M − H]⁻ | (400 MHz, DMSO-d6) δ 11.12 (s, 1H), 8.66- |
| | | | 8.33 (m, 2H), 8.19-8.01 (m, 2H), 7.66 (d, |
| | | | J = 8.6 Hz, 1H), 7.50-6.94 (m, 13H), 6.32- |
| | | | 6.04 (m, 2H), 5.42-5.33 (m, 1H), 5.25-4.63 |
| | | | (m, 3H), 4.49-0.86 (m, 48H), 0.84-0.73 (m, |
| | | | 3H). |
| 5.5 | 1201.2 | [M − H]⁻ | (400 MHz, DMSO-d6) δ 11.12 (s, 1H), 8.62- |
| | | | 8.34 (m, 2H), 8.18-8.03 (m, 2H), 7.68- |
| | | | 7.58 (m, 1H), 7.54-6.88 (m, 13H), 6.35- |
| | | | 6.13 (m, 2H), 5.43-5.33 (m, 1H), 5.25-4.64 |
| | | | (m, 3H), 4.50-0.93 (m, 48H), 0.93-0.79 (m, |
| | | | 3H). |
| 5.6 | 1283.2 | [M − H]⁻ | (400 MHz, DMSO-d6) δ 11.08 (s, 1H), 8.59- |
| | | | 8.26 (m, 2H), 8.08-7.79 (m, 2H), 7.65- |
| | | | 6.72 (m, 9H), 6.00-5.75 (m, 1H), 5.48-1.36 |
| | | | (m, 47H), 1.29-0.74 (m, 21H). |
| 5.7 | 1189.2 | [M + H]⁺ | (400 MHz, DMSO-d6) δ 11.09 (s, 1H), 8.61- |
| | | | 8.25 (m, 2H), 8.15-7.91 (m, 2H), 7.62 (d, J = |
| | | | 8.5 Hz, 1H), 7.47-7.24 (m, 7H), 7.18 (dd, |
| | | | J = 8.1, 6.3 Hz, 3H), 7.13-6.87 (m, 3H), 6.29- |
| | | | 6.05 (m, 2H), 5.43-5.26 (m, 1H), 5.24- |
| | | | 4.90 (m, 1H), 4.86-4.68 (m, 1H), 4.66 (m, |
| | | | 1H), 4.35 (m, 2H), 4.20-4.05 (m, 1H), 4.03- |
| | | | 3.96 (m, 2H), 3.93-3.51 (m, 2H), 3.50- |
| | | | 3.36 (m, 2H), 3.31 (s, 3H), 3.16-2.54 (m, |
| | | | 3H), 2.39 (m, 1H), 2.09-1.20 (m, 7H), 1.16- |
| | | | 1.11 (m, 4H), 1.09 (d, J = 7.1 Hz, 9H), 1.02 |
| | | | (s, 4H). |
| 5.8 | 1258.5 | [M + H]⁺ | (400 MHz, DMSO-d6) δ 11.09 (s, 1H), 8.58- |
| | | | 8.31 (m, 2H), 8.13-8.02 (m, 2H), 7.64-7.57 |
| | | | (m, 1H), 7.49-7.26 (m, 7H), 7.18 (dt, J = 7.4, |
| | | | 3.1 Hz, 3H), 7.12-6.88 (m, 3H), 6.34-6.06 |
| | | | (m, 2H), 5.34 (dd, J = 12.7, 5.4 Hz, 1H), 5.26- |
| | | | 4.90 (m, 1H), 4.85-4.70 (m, 1H), 4.66 (d, |
| | | | J = 10.2 Hz, 1H), 4.35 (m, 2H), 4.19-3.96 |
| | | | (m, 1H), 3.94-3.81 (m, 1H), 3.80-3.70 (m, |
| | | | 2H), 3.53 (s, 3H), 3.44 (m, 2H), 3.18-3.02 |
| | | | (m, 1H), 2.99-2.57 (m, 4H), 2.39 (m, 1H), |
| | | | 2.13-1.46 (m, 5H), 1.16-1.05 (m, 9H), 1.02 |
| | | | (s, 4H). |
| 5.9 | 1175.2 | [M + H]⁺ | (400 MHz, DMSO-d6) δ 11.09 (s, 1H), 8.64- |
| | | | 8.20 (m, 2H), 8.11-7.95 (m, 2H), 7.62 (d, J = |
| | | | 8.5 Hz, 1H), 7.49-7.25 (m, 8H), 7.20-6.88 |
| | | | (m, 7H), 6.54 (s, 1H), 6.32-5.93 (m, 2H), |
| | | | 5.45-5.25 (m, 1H), 5.18 (d, J = 8.1 Hz, 0.5H |
| | | | rotamer), 4.97 (d, J = 7.9 Hz, 0.5H rotamer), |
| | | | 4.84-4.51 (m, 2H), 4.35 (m, 2H), 4.20-3.95 |
| | | | (m, 1H), 3.93-3.63 (m, 2H), 3.56 (s, 1H), |
| | | | 3.50-3.37 (m, 5H), 3.14-2.99 (m, 1H), 2.96- |
| | | | 2.52 (m, 4H), 2.39 (m, 1H), 2.08-1.80 (m, |
| | | | 3H), 1.69 (m, 1H), 1.58 (d, J = 11.3 Hz, 1H), |
| | | | 1.13 (d, J = 7.1 Hz, 3H), 1.05 (m, 9H). |
| 5.10 | 1258.6 | [M + H]⁺ | (400 MHz, DMSO-d6) δ 11.13 (s, 1H), 8.82- |
| | | | 8.55 (m, 2H), 8.02-6.99 (m, 9H), 6.53 (s, |
| | | | 1H), 6.22-6.02 (m, 1H), 5.50-0.66 (m, |
| | | | 68H). |
| 5.11 | 1189.4 | [M + H]⁺ | (400 MHz, DMSO-d6) δ 11.09 (s, 1H), 8.53 |
| | | | (m, 1H), 8.50-8.41 (m, 1H), 8.37 (d, J = 8.7 |
| | | | Hz, 1H), 8.13-8.02 (m, 2H), 7.62 (d, J = 8.5 |
| | | | Hz, 1H), 7.49-7.27 (m, 8H), 7.22-6.85 (m, |
| | | | 7H), 6.26 (d, J = 6.2 Hz, 1H), 6.17-5.30 (m, |
| | | | 1H), 5.17 (d, J = 8.1 Hz, 0.5H), 4.97-3.97 |
| | | | (m, 7H), 3.89 (m, 3H), 3.83-3.68 (m, 2H), |
| | | | 3.45 (m, 3H), 3.31 (s, 3H), 3.18-3.05 (m, |
| | | | 1H), 2.99-2.57 (m, 5H), 2.36 (m, 1H), 2.11- |
| | | | 1.48 (m, 6H), 1.39-1.18 (m, 1H), 1.12 (d, |
| | | | J = 7.1 Hz, 3H), 1.07 (d, J = 9.5 Hz, 8H), 1.05- |
| | | | 0.95 (m, 8H). |
| 5.12 | 1203.6 | [M + H]⁺ | (400 MHz, DMSO) δ 11.09 (s, 1H), 8.63- |
| | | | 8.28 (m, 2H), 8.12-7.97 (m, 2H), 7.62 (d, J = |
| | | | 8.5 Hz, 1H), 7.49-7.25 (m, 8H), 7.19 (d, J = |
| | | | 7.8 Hz, 4H), 7.11-6.86 (m, 3H), 6.29-6.10 |
| | | | (m, 2H), 5.34 (dd, J = 12.7, 5.4 Hz, 1H), 5.28- |
| | | | 4.22 (m, 6H), 4.22-3.39 (m, 6H), 3.21- |
| | | | 3.01 (m, 1H), 2.97-2.53 (m, 5H), 2.16-1.41 |
| | | | (m, 5H), 1.33-1.11 (m, 6H), 1.10 (d, J = 9.2 |
| | | | Hz, 9H), 1.02 (s, 5H). |
| 5.13 | 1203.2 | [M + H]⁺ | (400 MHz, DMSO-d6) δ 11.09 (s, 1H), 8.53 |
| | | | (m,1H), 8.48-8.00 (m, 3H), 7.62 (d, J = 8.5 |
| | | | Hz, 1H), 7.48-7.25 (m, 8H), 7.21-7.13 (m, |
| | | | 4H), 7.11-6.89 (m, 3H), 6.35-6.04 (m, 1H), |
| | | | 6.08 (m, 1H), 5.43-5.29 (m, 1H), 5.27-4.86 |
| | | | (m, 1H), 4.83-4.68 (m, 2H), 4.66 (m, 1H), |
| | | | 4.38 (m, 1H), 4.32 (m, 1H), 4.26-3.79 (m, |
| | | | 2H), 3.78-3.50 (m, 2H), 3.44 (m, 2H), 3.31 |
| | | | (s, 2H), 3.15-3.03 (m, 1H), 2.95-2.56 (m, |
| | | | 5H), 2.36 (m, 1H), 2.11-1.75 (m, 3H), 1.64 |
| | | | (m, 2H), 1.12 (s, 2H), 1.11-1.05 (m, 10H), |
| | | | 1.04-0.84 (m, 8H). |
| 5.14 | 1221.4 | [M + H]⁺ | (400 MHz, DMSO-d6) δ 11.13 (s, 1H), 9.36- |
| | | | 8.40 (m, 2H), 8.10-6.13 (m, 19H), 5.42 (d, J = |
| | | | 12.5 Hz, 1H), 5.23 (d, J = 8.0 Hz, 1H), 5.07- |
| | | | 0.51 (m, 51H). |
| 5.15 | 1169.4 | [M − H]⁻ | (400 MHz, DMSO-d6) δ 11.09 (s, 1H), 9.36 |
| | | | (s, 1H), 9.03 (s, 1H), 8.52-8.28 (m, 1H), 8.08- |
| | | | 7.83 (m, 2H), 7.60-7.23 (m, 4H), 7.20- |
| | | | 7.11 (m, 1H), 7.05-6.98 (m, 2H), 6.88-6.72 |
| | | | (m, 3H), 5.50-5.26 (m, 3H), 4.82-4.69 (m, |
| | | | 1H), 4.48-3.82 (m, 12H), 3.08 (m, 2H), 3.05- |
| | | | 2.62 (m, 6H), 2.21-1.98 (m, 2H), 1.65-1.45 |
| | | | (m, 4H), 1.29-0.81 (m, 22H), 0.89-0.82 (m, |
| | | | 4H). |
| 5.16 | 1125.4 | [M − H]⁻ | (400 MHz, DMSO-d6) δ 11.12 (s, 1H), 8.62- |
| | | | 8.34 (m, 2H), 8.18-8.03 (m, 2H), 7.68-7.58 |
| | | | (m, 1H), 7.54-6.88 (m, 13H), 6.35-6.13 (m, |
| | | | 2H), 5.43-5.33 (m, 1H), 5.25-4.64 (m, 3H), |
| | | | 4.50-0.93 (m, 48H), 0.93-0.79 (m, 3H). |

### Example 6.1: ((2-(((2S)-1-((2S,3R)-3-(3-((1r,4R)-4-((1-(2,6-dioxopiperidin-3-yl)-3-methyl-2-oxo-2,3-dihydro-1H-benzo[d]imidazol-5-yl)methyl)cyclohexyl)propanamido)-2-((R)-2-phenylmorpholine-4-carbonyl)pyrrolidin-1-yl)-3,3-dimethyl-1-oxobutan-2-yl)carbamoyl)benzo[b]thiophen-5-yl)difluoromethyl)phosphonic acid

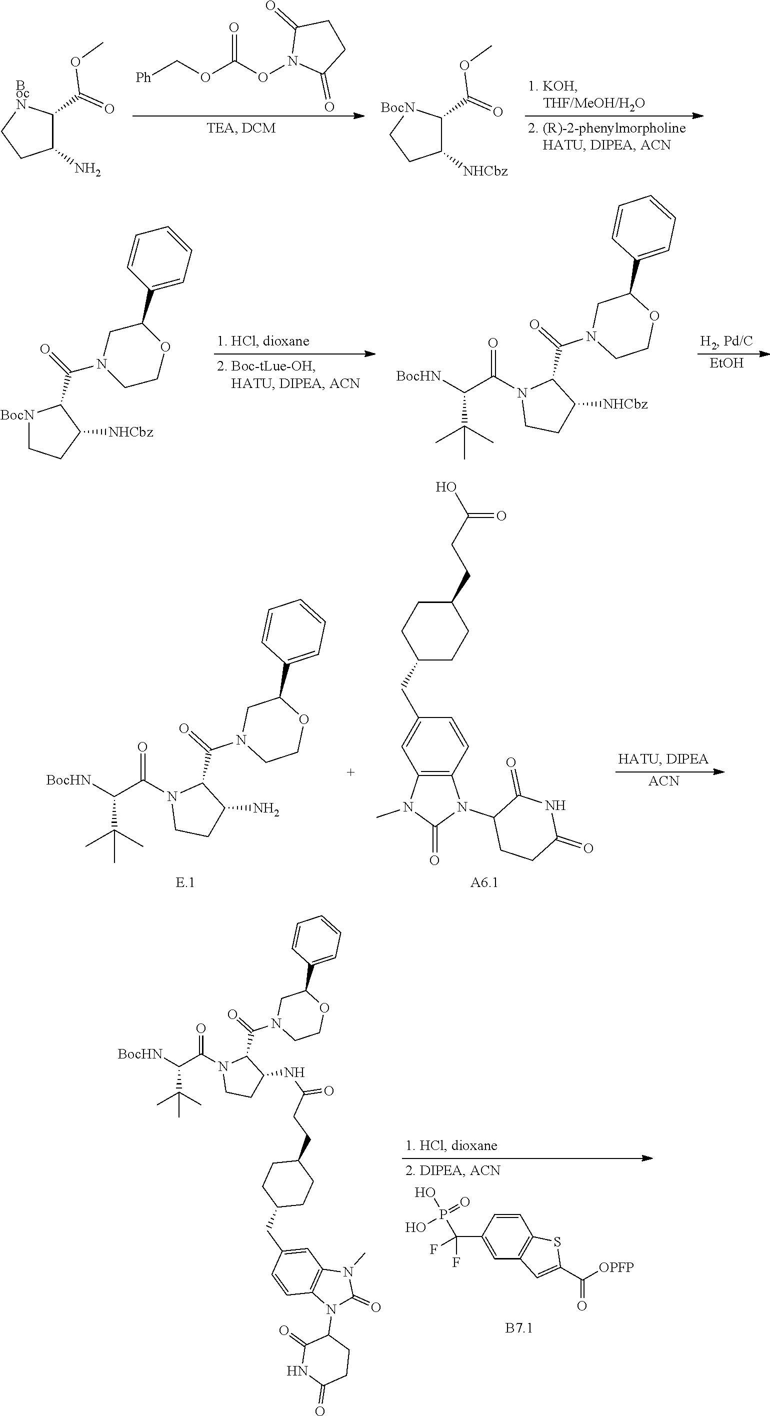

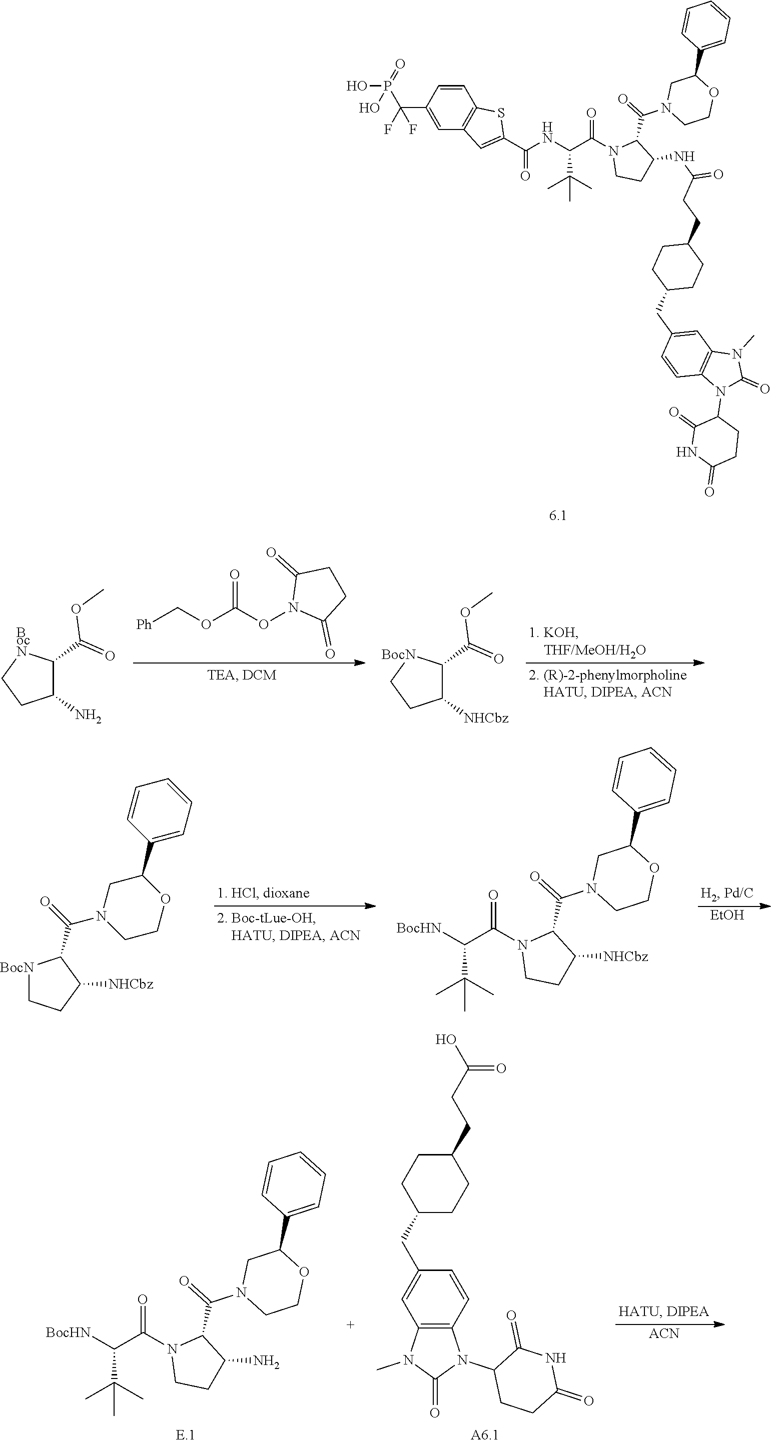

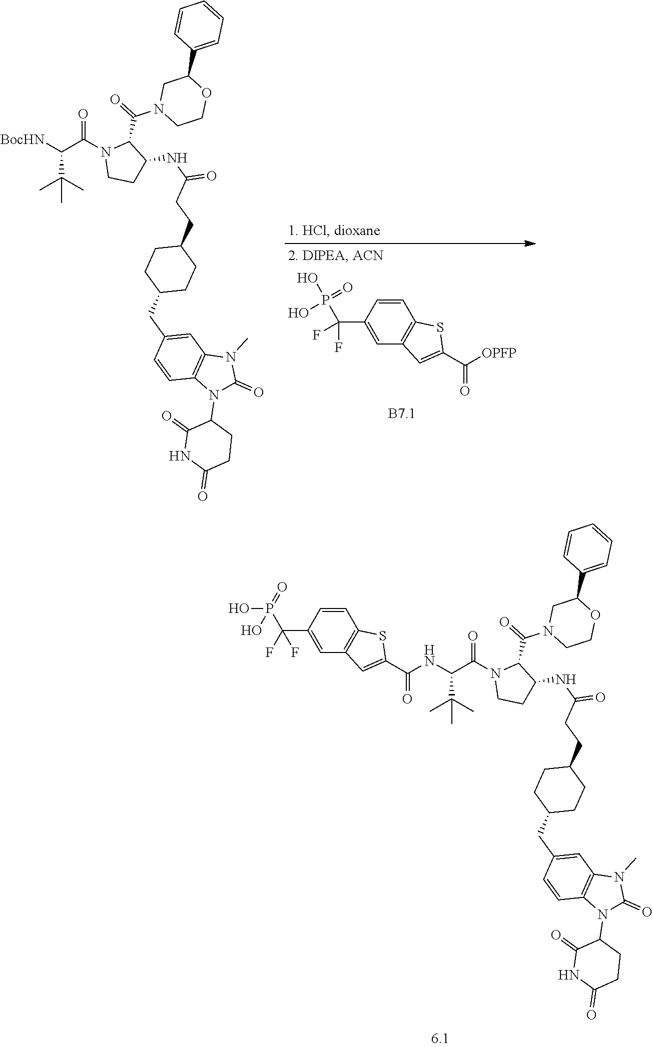

1-(tert-butyl) 2-methyl (2S,3R)-3-(((benzyloxy)carbonyl)amino)pyrrolidine-1,2-dicarboxylate. To a solution of 1-(tert-butyl) 2-methyl (2S,3R)-3-aminopyrrolidine-1,2-dicarboxylate hydrochloride (1.00 g, 3.56 mmol) in 15 mL DCM was added N(Benzyloxycarbonyloxy) succinimide (1065 mg, 4.27 mmol) followed by TEA (1395 μL, 7.84 mmol). After 2 h, the reaction was concentrated and the crude product was purified by silica gel column chromatography eluting with MeOH and DCM to afford the title compound. ES/MS: m/z=401 [M+Na]⁺.

(2S,3R)-3-(((benzyloxy)carbonyl)amino)-1-(tert-butoxycarbonyl)pyrrolidine-2-carboxylic acid. 1-(tert-butyl) 2-methyl (2S,3R)-3-(((benzyloxy)carbonyl)amino)pyrrolidine-1,2-dicarboxylate (1.1 g, 2.9 mmol) was dissolved in 12 mL 1:1 THF:MeOH. potassium hydroxide (1.0 mol/L, 12 mL, 12 mmol) in water was added to the solution and stirred at RT. After 4 h, the organics were distilled off and 12 mL 1N hydrochloric acid was added. The mixture was then frozen and lyophilized to afford the title compound which was used without further purification. ES/MS: m/z=387.1 [M+Na]⁺.

tert-butyl (2S,3R)-3-(((benzyloxy)carbonyl)amino)-2-((R)-2-phenylmorpholine-4-carbonyl)pyrrolidine-1-carboxylate. (2S,3R)-3-(((benzyloxy)carbonyl)amino)-1-(tertbutoxycarbonyl)pyrrolidine-2-carboxylic acid (2.9 mmol) was dissolved in 15 mL ACN. (2R)-2-phenylmorpholine (710 mg, 4.3 mmol), HATU (819 mg, 2.2 mmol), and then Hunig's base (1254 μL, 7.3 mmol) were added to the mixture. After 10 minutes reaction was filtered and the filtrated was concentrated under reduced pressure. The crude material was purified by silica gel column chromatography eluting with MeOH and EA to afford the title compound. ES/MS: m/z=532.2 [M+Na]⁺. 1H NMR (400 MHz, CDCl₃) δ 7.48-7.32 (m, 10H), 5.13-4.84 (m, 2H), 4.69-4.31 (m, 3H), 4.21-3.59 (m, 4H), 3.55-3.29 (m, 2H), 2.98-2.57 (m, 2H), 2.43-2.16 (m, 2H), 1.62-1.37 (m, 9H).

benzyl ((2S,3R)-2-((R)-2-phenylmorpholine-4-carbonyl)pyrrolidin-3-yl)carbamate HCl. Tert-butyl (2S,3R)-3-(((benzyloxy)carbonyl)amino)-2-((R)-2-phenylmorpholine-4-carbonyl)pyrrolidine-1-carboxylate (1.20 g, 2.35 mmol) was dissolved in 4.0M HCl in 1,4-dioxane (12.0 mL) and stirred RT. After 30 minutes, the reaction was frozen and lyophilized to afford the title compound which was used without further purification. ES/MS: m/z=410.2 [M+H]⁺.

benzyl ((2S,3R)-1-((S)-2-((tert-butoxycarbonyl)amino)-3,3-dimethylbutanoyl)-2-((R)-2-phenylmorpholine-4-carbonyl)pyrrolidin-3-yl)carbamate. Benzyl ((2S,3R)-2-((R)-2-phenylmorpholine-4-carbonyl)pyrrolidin-3-yl)carbamate HCl (1.1 g, 2.4 mmol) was dissolve in 10 mL ACN. Boc-L-Tle-OH (660 mg, 2.9 mmol), HATU (671 mg, 1.8 mmol), and then Hunig's base (1028 μL, 5.9 mmol) were added to reaction. After 20 minutes, the reaction was concentrated under reduced pressure. The crude material was purified by silica gel column chromatography eluting with EA and hexanes to afford the title compound. ES/MS: m/z=623.3 [M+H]⁺. 1H NMR (400 MHz, CDCl3) δ 7.49-7.29 (m, 10H), 5.35-4.95 (m, 3H), 4.70-4.22 (m, 4H), 4.08-3.79 (m, 3H), 3.73-3.39 (m, 2H), 3.03-2.59 (m, 2H), 2.43-2.20 (m, 2H), 1.46 (d, J=8.6 Hz, 9H), 1.02 (d, J=18.3 Hz, 9H).

tert-butyl ((S)-1-((2S,3R)-3-amino-2-((R)-2-phenylmorpholine-4-carbonyl)pyrrolidin-1-yl)-3,3-dimethyl-1-oxobutan-2-yl)carbamate (E1.1). Benzyl ((2S,3R)-1-((S)-2-((tert-butoxycarbonyl)amino)-3,3-dimethylbutanoyl)-2-((R)-2-phenylmorpholine-4-carbonyl)pyrrolidin-3-yl)carbamate (700 mg, 1.12 mmol) was dissolved in EtOH. ˜30 mg of 10% Pd/C was added to reaction The reaction was stirred under 1 atm. H₂ for 16 h. The reaction was filtered and the filtrate was concentrated to E1.1 which was used without further purification. ES/MS: m/z=489.3 [M+H]⁺.

tert-butyl ((2S)-1-((2S,3R)-3-(3-((1r,4R)-4-((1-(2,6-dioxopiperidin-3-yl)-3-methyl-2-oxo-2,3-dihydro-1H-benzo[d]imidazol-5-yl)methyl)cyclohexyl)propanamido)-2-((R)-2-phenylmorpholine-4-carbonyl)pyrrolidin-1-yl)-3,3-dimethyl-1-oxobutan-2-yl)carbamate. Tert-butyl ((S)-1-((2S,3R)-3-amino-2-((R)-2-phenylmorpholine-4-carbonyl)pyrrolidin-1-yl)-3,3-dimethyl-1-oxobutan-2-yl)carbamate CA9114-A (73 mg, 0.15 mmol) was in 1 mL ACN. A6.1 (66 mg, 0.15 mmol), HATU (42 mg, 0.11 mmol), and then Hunig's base (104 μL, 0.60 mmol) were added. After 10 minutes, the mixture was concentrated and the crude material was purified by silica gel column chromatography eluting with MeOH and DCM and then purified by reverse phase silica gel and then HPLC chromatography eluting with ACN with 0.1% TFA and water with 0.1% TFA affording the title compound. ES/MS: m/z=920.3 [M+Na]⁺.

1H NMR (400 MHz, CDCl3) δ 8.38-8.29 (m, 1H), 7.45 (dt, J=8.0, 1.5 Hz, 1H), 7.42-7.30 (m, 4H), 6.90-6.78 (m, 2H), 6.76-6.68 (m, 1H), 6.32-6.13 (m, 1H), 5.46-5.06 (m, 4H), 4.74-4.56 (m, 2H), 4.50 (d, J=13.3 Hz, 1H), 4.37-4.23 (m, 2H), 4.15-4.03 (m, 1H), 4.02-3.77 (m, 3H), 3.73-3.49 (m, 2H), 3.20-2.64 (m, 5H), 2.53 (t, J=6.3 Hz, 2H), 2.39-2.01 (m, 5H), 1.79-1.64 (m, 4H), 1.46 (d, J=9.6 Hz, 12H), 1.03 (d, J=22.3 Hz, 13H).

N-((2S,3R)-1-((S)-2-amino-3,3-dimethylbutanoyl)-2-((R)-2-phenylmorpholine-4-carbonyl)pyrrolidin-3-yl)-3-((1r,4R)-4-((1-(2,6-dioxopiperidin-3-yl)-3-methyl-2-oxo-2,3-dihydro-1H-benzo[d]imidazol-5-yl)methyl)cyclohexyl)propenamide HCl. Tert-butyl ((2S)-1-((2S,3R)-3-(3-((1r,4R)-4-((1-(2,6-dioxopiperidin-3-yl)-3-methyl-2-oxo-2,3-dihydro-1H-benzo[d]imidazol-5-yl)methyl)cyclohexyl)propanamido)-2-((R)-2-phenylmorpholine-4-carbonyl)pyrrolidin-1-yl)-3,3-dimethyl-1-oxobutan-2-yl)carbamate (90.0 mg, 0.100 mmol) was dissolved in 4.0M HCl in 1,4-dioxane (4 mL) and stirred RT. After 30 minutes, the mixture was frozen and lyophilized, affording the title compound. ES/MS: m/z=798.4 [M+H]⁺.

((2-(((2S)-1-((2S,3R)-3-(3-((1r,4R)-4-((1-(2,6-dioxopiperidin-3-yl)-3-methyl-2-oxo-2,3-dihydro-1H-benzo[d]imidazol-5-yl)methyl)cyclohexyl)propanamido)-2-((R)-2-phenylmorpholine-4-carbonyl)pyrrolidin-1-yl)-3,3-dimethyl-1-oxobutan-2-yl)carbamoyl)benzo[b]thiophen-5-yl)difluoromethyl)phosphonic acid. N-((2S,3R)-1-((S)-2-amino-3,3-dimethylbutanoyl)-2-((R)-2-phenylmorpholine-4-carbonyl)pyrrolidin-3-yl)-3-((1r,4R)-4-((1-(2,6-dioxopiperidin-3-yl)-3-methyl-2-oxo-2,3-dihydro-1H-benzo[d]imidazol-5-yl)methyl)cyclohexyl)propenamide (42.2 mg, 0.0506 mmol) and [difluoro-[2-(2,3,4,5,6-pentafluorophenoxy)carbonylbenzothiophen-5-yl]methyl]phosphonic acid B7.1 (20.0 mg, 0.0422 mmol) were dissolved in 1 mL ACN. Hunig's base (36.5 μL, 0.211 mmol) was added and the reaction was stirred at RT. After 30 minutes, the reaction was purified by reverse phase chromatography eluting with ACN with 0.1% TEA and water with 0.1% TEA affording the title compound.

TABLE 6.1
ES/MS and 1H NMR data for Example 6.1

| Example | ES/MS | | |
|---|---|---|---|
| Number | m/z | Ion | 1H NMR |
| 6.1 | 1086.2 | [M − H]⁻ | (400 MHz, DMSO) δ 11.08 (s, 1H), 8.61- |
| | | | 8.51 (m, 1H), 8.50-8.31 (m, 1H), 8.15 (d, J = |
| | | | 8.4 Hz, 1H), 8.12-7.97 (m, 2H) 7.59 (d, J = |
| | | | 8.7 Hz, 1H), 7.33 (d, J = 7.5 Hz, 4H), 7.03- |
| | | | 6.94 (m, 2H), 6.85-6.76 (m, 1H), * 5.37- |
| | | | 5.29 (m, 1H), 5.22 (d, J = 7.9 Hz, 1H), 5.03- |
| | | | 4.71 (m, 2H), 4.61-4.46 (m, 2H), 4.39-4.21 |
| | | | (m, 3H), 4.17-2.81 (m, 12H), 2.80-2.56 (m, |
| | | | 4H), 2.35-1.86 (m, 5H), 1.74-1.52 (m, 3H), |
| | | | 1.54-1.19 (m, 4H), 1.18-0.98 (m, 9H), 0.98- |
| | | | 0.70 (m, 4H). |

### V. Biological Examples

### Buffers and Reagents

The following buffers and reagents were used in the assays:

| Reagent | Source | Catalog # |
|---|---|---|
| Cytofix fixation buffer | BD Biosciences | 554655 |
| Anti-human STAT6 mAb, clone 3 | Invitrogen | MA5-29583 |
| Alexa Fluor 647 antibody | Invitrogen | A20186 |
| labeling kit | | |
| Perm/Wash Buffer | BD Biosciences | 554723 |
| DyLight ™ 800 NHS Ester | Invitrogen | 46421 |
| PE/Cyanine7 anti-human CD3 | Biolegend | 317334 |
| Antibody | | |
| Brilliant Violet 650 ™ | Biolegend | 317436 |
| anti-human CD4 Antibody | | |
| Wash buffer | 3% FBS in 1x DPBS | NA |

### Example A

The human peripheral blood mononuclear cell (PBMC) STAT6 flow cytometry assay was used to determine the concentration of tested compounds that degrades 50% of total STAT6 protein (DC₅₀; provided in Table 7 as STAT6 PBMC DC₅₀) as well as to determine the maximal degree of STAT6 degradation achievable for a given compound (Dₘₐₓ).

In this assay, anti-STAT6 antibodies were used to measure total STAT6 protein levels in CD3⁺CD4⁺ T cell subpopulation in PBMCs by flow cytometry.

Test compounds dissolved in DMSO at various concentrations were first spotted in assay plates using a Labcyte Echo acoustic dispenser. Cryopreserved PBMCs were then seeded at 50,000 cells/well in the assay plates using an Agilent EL406 dispenser and incubated with test compounds for 24 hours at 5% CO₂ in a 37° C. incubator. After treatment, the cells were washed twice with wash buffer and stained with DyLight™ 800 at 5 μg/mL and Fc receptor blocker at 1:100 dilution for 15 minutes. All the procedures thereafter were carried out at room temperature. Cells were washed twice with wash buffer, then fixed with 2% paraformaldehyde for 10 minutes. Cells were washed three times with Perm/Wash buffer, permeabilized with Perm/Wash buffer for 1 hour, and stained with antibodies (anti-CD3 and anti-CD4 Ab at 1:100 dilution; anti-STAT6 Ab at 1:2000 dilution) for another 1 hour. Cells were washed in wash buffer 5 times followed by fixation with 0.5% paraformaldehyde. Fifteen minutes later, paraformaldehyde was removed by washes with PBS and the cells were analyzed by flow cytometry using an Attune NxT instrument. The median fluorescent intensity (MFI) of STAT6 in CD3⁺CD4⁺ T cells for each of the treatment conditions was normalized to vehicle control (DMSO, 0% degradation) and 1 μM of an internal control compound (100% degradation) which was independently confirmed to have >95% protein degradation by Western Blot analysis. Least squares curve fittings were performed using a four-parameter variable slope nonlinear regression model to obtain DC₅₀. The lowest mean values in the curve represented Dₘₐₓ.

TABLE 7
Assay Results

| Example | STAT6 PBMC | Dₘₐₓ |
|---|---|---|
| Number | DC₅₀ (nM) | (%) |
| 1.1 | 5 | >95 |
| 1.2 | 13 | >95 |
| 1.3 | 6 | >95 |
| 1.4 | 7 | >95 |
| 1.5 | 44 | >95 |
| 1.6 | 23 | >95 |
| 1.7 | 28 | >95 |
| 1.8 | 21 | >95 |
| 1.9 | 6 | >95 |
| 1.10 | 17 | >95 |
| 1.11 | 52 | >95 |
| 1.12 | 31 | >95 |
| 1.13 | 39 | >95 |
| 1.14 | 18 | >95 |
| 1.15 | 19 | >95 |
| 1.16 | 16 | >95 |
| 1.17 | 44 | >95 |
| 1.18 | 595 | 84 |
| 1.19 | 7 | >95 |
| 1.20 | 9 | 88 |
| 1.21 | 9 | >95 |
| 1.22 | 10 | >95 |
| 1.23 | 11 | 94 |
| 1.24 | 15 | >95 |
| 1.25 | 15 | >95 |
| 1.26 | 18 | >95 |
| 1.27 | 19 | >95 |
| 1.28 | 13 | >95 |
| 1.29 | 32 | >95 |
| 1.30 | 37 | 83 |
| 1.31 | 40 | >94 |
| 1.32 | 45 | 77 |
| 1.33 | 50 | 91 |
| 1.34 | 55 | 93 |
| 1.35 | 55 | 87 |
| 1.36 | 64 | 89 |
| 1.37 | 70 | 87 |
| 1.38 | 70 | 88 |
| 1.39 | 75 | 89 |
| 1.40 | 84 | >95 |
| 1.41 | 103 | 78 |
| 1.44 | 7 | >95 |
| 1.45 | 6 | >95 |
| 1.46 | 43 | >95 |
| 2.1 | 10 | >95 |
| 2.2 | 10 | >95 |
| 2.3 | 28 | 90 |
| 3.1 | 19 | >95 |
| 3.2 | 67 | >95 |
| 3.3 | 21 | >95 |
| 3.4 | 12 | >95 |
| 3.5 | 16 | >95 |
| 3.6 | 21 | >95 |
| 3.7 | 37 | 88 |
| 3.8 | 82 | 88 |
| 3.9 | 100 | 49 |
| 3.11 | 21 | >95 |
| 3.12 | 44 | 93 |
| 3.13 | 33 | >95 |
| 4.1 | 19 | >95 |
| 5.1 | <0.5 | >95 |
| 5.2 | <0.5 | >95 |
| 5.3 | <0.5 | >95 |
| 5.4 | <0.5 | >95 |
| 5.5 | <0.5 | >95 |
| 5.6 | <0.5 | >95 |
| 5.7 | <0.5 | >95 |
| 5.8 | <0.5 | >95 |
| 5.9 | <0.5 | >95 |
| 5.10 | <0.5 | >95 |
| 5.11 | <0.5 | >95 |
| 5.12 | <0.5 | >95 |
| 5.13 | <0.5 | >95 |
| 5.14 | <0.5 | >95 |
| 5.15 | 17 | 93 |
| 6.1 | 38 | >95 |

The present disclosure provides reference to various embodiments and techniques. However, it should be understood that many variations and modifications can be made while remaining within the spirit and scope of the present disclosure. The description is made with the understanding that it is to be considered an exemplification of the claimed subject matter, and is not intended to limit the appended claims to the specific embodiments illustrated.

1. A compound of Formula J 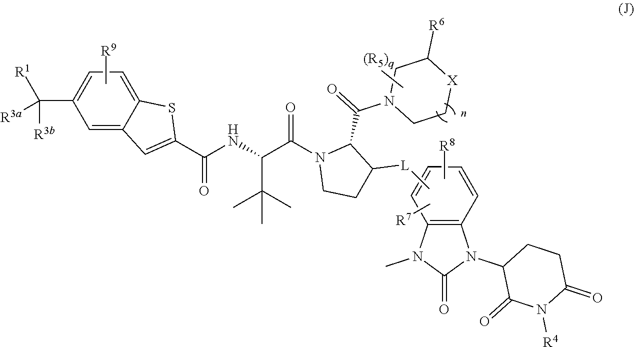
or a pharmaceutically acceptable salt thereof, wherein
R¹ is 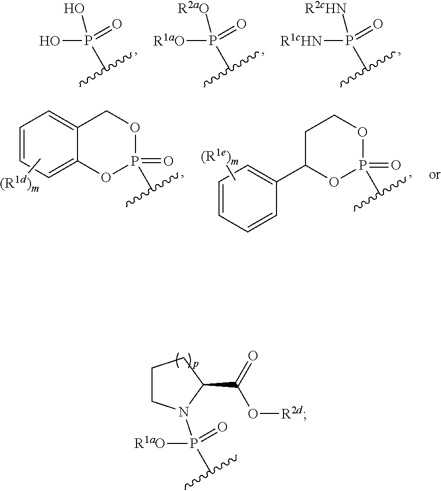
R^{1a} and R^{2a} are each independently C₁₋₆ alkyl, C₁₋₆ haloalkyl, C₁₋₆ alkylene-OC(O)—C₁₋₆ alkyl, C₁₋₆ alkylene-OC(O)O—C₁₋₆ alkyl, C₁₋₆ alkylene-SC(O)—C₁₋₆ alkyl, C₃₋₆ cycloalkyl, C₆₋₁₂ aryl, or 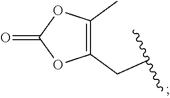 [SMILES: *Cc1oc(=O)oc1C]
R^{1b} is H, C₁₋₆ alkyl, C₁₋₆ haloalkyl, C₃₋₆ cycloalkyl or C₆₋₁₂ aryl;
R^{1c}, R^{2b}, and R^{2c} are each independently 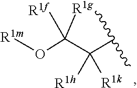
wherein
R^{1f} and R^{1g} are each independently H or C₁₋₆ alkyl, or R^{1f} and R^{1g} together are oxo,
R^{1h} and R^{1k}, are each independently H, C₁₋₆ alkyl, C₁₋₆ haloalkyl, C₂₋₆ alkoxyalkyl, or CH₂Ph, and
R^{1m} is C₁₋₆ alkyl, C₁₋₆ haloalkyl, C₂₋₆ alkoxyalkyl, —C(O)—C₁₋₆ alkyl, —C(O)O—C₁₋₆ alkyl, C₃₋₈ cycloalkyl, —CH₂—C₃₋₈ cycloalkyl, heterocycloalkyl, or —CH₂-heterocycloalkyl, wherein each heterocycloalkyl has 3 to 8 ring members and 1 to 3 heteroatoms each independently N, O, or S, provided that when R^{1m} is —C(O)C₁₋₆ alkyl or —C(O)O—C₁₋₆ alkyl, then R^{1f} and R^{1g} are each independently H or C₁₋₆ alkyl;
R^{1d} and R^{1e} are each independently C₁₋₆ alkyl, C₁₋₆ alkoxy, C₂₋₆ alkoxyalkyl, halogen, C₁₋₆ haloalkyl, C₁₋₆ haloalkoxy, or CN;
R^{2d} is C₁₋₆ alkyl or C₁₋₆ haloalkyl;
R^{3a} and R^{3b} are each independently H, C₁₋₆ alkyl, halogen, or C₁₋₆ haloalkyl, or R^{3a} and R^{3b} together are oxo;
L is -L¹-L²-L³-L⁴-L⁵-;
L¹ is absent, CH₂, O, or N(L^{1a});
L^{1a} is hydrogen or C₁₋₃ alkyl;
L² is absent, C₁₋₆ alkylene, C₁₋₆ alkylene-C(O), CH₂C(O)N(L^{2a}), CH₂C(O)N(L^{2a})-C₁₋₆ alkylene, —C(O)—, or —C(O)—C₁₋₆ alkylene, provided that when L² is —C(O)— or —C(O)—C₁₋₆ alkylene, then L¹ is absent, CH₂, or N(L^{1a});
L^{2a} is hydrogen or C₁₋₆ alkyl;
L³ is absent, C₃₋₈ cycloalkylene, or a heterocycloalkylene having 4 to 11 ring members and 1 to 3 heteroatoms each independently N, O, or S, wherein each cycloalkylene and heterocycloalkylene is substituted with 0, 1, 2, or 3 R^{L3};
L⁴ is absent, C₁₋₆ alkylene, C₂₋₆ alkenylene, C₂₋₆ alkynylene, O, or C(O), wherein the alkylene is substituted with 0, 1, 2, or 3 R^{L4};
L⁵ is absent, C₁₋₆ alkylene, C₃₋₈ cycloalkylene, or a heterocycloalkylene having 4 to 11 ring members and 1 to 3 heteroatoms each independently N, O, or S, wherein each alkylene, cycloalkylene and heterocycloalkylene is substituted with 0, 1, 2, or 3 R^{L5}, and wherein no more than three of L¹, L², L³, L⁴, and L⁵ are absent;
each R^{L3}, R^{L4} and R^{L5} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
R⁴ is hydrogen, CH₂OC(O)—C₁₋₆ alkyl, CH₂OC(O)O—C₁₋₆ alkyl, or CH₂—OP(O)(OH)₂;
each R⁵ is independently halo, C₁₋₆ alkyl, or C₁₋₆ haloalkyl;
alternatively, where two R⁵ groups are bound to the same ring atom, such R⁵ groups can be taken together with the atom to which they are attached to form a C₃₋₅ cycloalkyl;
R⁶ is hydrogen, C₁₋₆ alkyl, C₁₋₆ alkoxy, C₆₋₁₀ aryl, heteroaryl having 5 to 10 ring members and 1 to 3 heteroatoms each independently N, O, or S, or —OR^{6a}, wherein the aryl and heteroaryl is substituted with 0, 1, 2, or 3 R^{6b};
R^{6a} is C₆₋₁₀ aryl or heteroaryl having 5 to 10 ring members and 1 to 3 heteroatoms each independently N, O, or S, wherein the aryl and heteroaryl is substituted with 0, 1, 2, or 3 R^{6c};
each R^{6b} and R^{6c} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
X is —CH₂—, —CF₂—, —CH(CN)—, —O—, or —N(R^{X})—;
R^{X} is hydrogen or C₁₋₆ alkyl;
subscript m is 0, 1, 2, or 3;
subscript n is 0, 1, or 2, provided that when subscript n is 0, then X is —CH₂— or —CH(CN)—, and if subscript n is 1 or 2 then X is —O—, —CH₂—, or —N(R^{X});
subscript p is 0, 1, or 2;
subscript q is 0, 1, or 2; and
R⁷, R⁸, and R⁹ are each independently hydrogen or fluorine.

2. The compound of claim 1, which is a compound of Formula I: 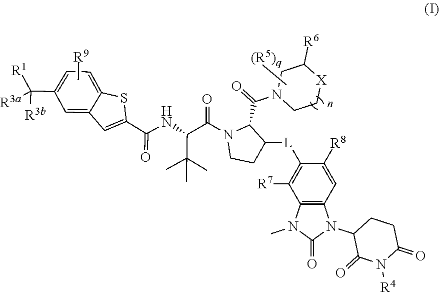
or a pharmaceutically acceptable salt thereof, wherein
R¹ is 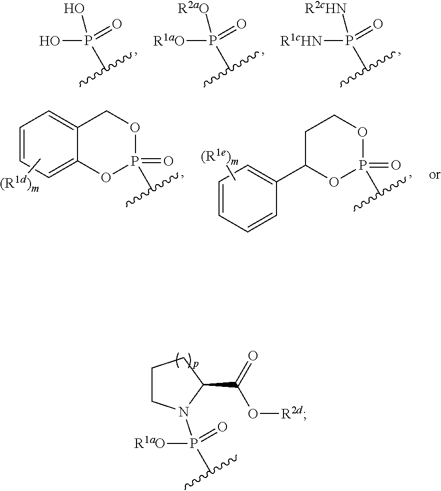
R^{1a} and R^{2a} are each independently C₁₋₆ alkyl, C₁₋₆ haloalkyl, C₁₋₆ alkylene-OC(O)—C₁₋₆ alkyl, C₁₋₆ alkylene-OC(O)O—C₁₋₆ alkyl, C₁₋₆ alkylene-SC(O)—C₁₋₆ alkyl, C₃₋₆ cycloalkyl, C₆₋₁₂ aryl, or 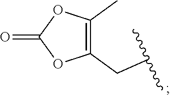 [SMILES: *Cc1oc(=O)oc1C]
R^{1b} is H, C₁₋₆ alkyl, C₁₋₆ haloalkyl, C₃₋₆ cycloalkyl or C₆₋₁₂ aryl;
R^{1c}, R^{2b}, and R^{2c} are each independently 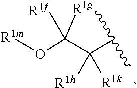
wherein
R^{1f} and R^{1g} are each independently H or C₁₋₆ alkyl, or R^{1f} and R^{1g} together are oxo,
R^{1h} and R^{1k}, are each independently H, C₁₋₆ alkyl, C₁₋₆ haloalkyl, C₂₋₆ alkoxyalkyl, or CH₂Ph, and
R^{1m} is C₁₋₆ alkyl, C₁₋₆ haloalkyl, C₂₋₆ alkoxyalkyl, —C(O)—C₁₋₆ alkyl, —C(O)O—C₁₋₆ alkyl, C₃₋₈ cycloalkyl, —CH₂—C₃₋₈ cycloalkyl, heterocycloalkyl, or —CH₂-heterocycloalkyl, wherein each heterocycloalkyl has 3 to 8 ring members and 1 to 3 heteroatoms each independently N, O, or S, provided that when R^{1m} is —C(O)C₁₋₆ alkyl or —C(O)O—C₁₋₆ alkyl, then R^{1f} and R^{1g} are each independently H or C₁₋₆ alkyl;
R^{1d} and R^{1e} are each independently C₁₋₆ alkyl, C₁₋₆ alkoxy, C₂₋₆ alkoxyalkyl, halogen, C₁₋₆ haloalkyl, C₁₋₆ haloalkoxy, or CN;
R^{2d} is C₁₋₆ alkyl or C₁₋₆ haloalkyl;
R^{3a} and R^{3b} are each independently H, C₁₋₆ alkyl, halogen, or C₁₋₆ haloalkyl, or R^{3a} and R^{3b} together are oxo;
L is -L¹-L²-L³-L⁴-L⁵-;
L¹ is absent, CH₂, O, or N(L^{1a});
L^{1a} is hydrogen or C₁₋₃ alkyl;
L² is absent, C₁₋₆ alkylene, CH₂C(O), CH₂C(O)N(L^{2a}), CH₂C(O)N(L^{2a})-C₁₋₆ alkylene, —C(O)—, or —C(O)—C₁₋₆ alkylene, provided that when L² is —C(O)— or —C(O)—C₁₋₆ alkylene, then L¹ is absent, CH₂, or N(L^{1a});
L^{2a} is hydrogen or C₁₋₆ alkyl;
L³ is absent, C₃₋₈ cycloalkylene, or a heterocycloalkylene having 4 to 11 ring members and 1 to 3 heteroatoms each independently N, O, or S, wherein each cycloalkylene and heterocycloalkylene is substituted with 0, 1, 2, or 3 R^{L3};
L⁴ is absent, C₁₋₆ alkylene, C₂₋₆ alkenylene, C₂₋₆ alkynylene, O, or C(O), wherein the alkylene is substituted with 0, 1, 2, or 3 R^{L4};
L⁵ is absent, C₁₋₆ alkylene, C₃₋₈ cycloalkylene, or a heterocycloalkylene having 4 to 11 ring members and 1 to 3 heteroatoms each independently N, O, or S, wherein each alkylene, cycloalkylene and heterocycloalkylene is substituted with 0, 1, 2, or 3 R^{L5}, and wherein no more than two of L¹, L², L³, L⁴, and L⁵ are absent;
each R^{L3}, R^{L4} and R^{L5} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
R⁴ is hydrogen, CH₂OC(O)—C₁₋₆ alkyl, CH₂OC(O)O—C₁₋₆ alkyl, or CH₂—OP(O)(OH)₂;
each R⁵ is independently C₁₋₆ alkyl, or C₁₋₆ haloalkyl;
alternatively, where two R⁵ groups are bound to the same ring atom, such R⁵ groups can be taken together with the atom to which they are attached to form a C₃₋₅ cycloalkyl;
R⁶ is hydrogen, C₁₋₆ alkyl, C₁₋₆ alkoxy, C₆₋₁₀ aryl, heteroaryl having 5 to 10 ring members and 1 to 3 heteroatoms each independently N, O, or S, or —OR^{6a}, wherein the aryl and heteroaryl is substituted with 0, 1, 2, or 3 R^{6b};
R^{6a} is C₆₋₁₀ aryl or heteroaryl having 5 to 10 ring members and 1 to 3 heteroatoms each independently N, O, or S, wherein the aryl and heteroaryl is substituted with 0, 1, 2, or 3 R^{6c};
each R^{6b} and R^{6c} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
X is —CH₂—, —CH(CN)—, —O—, or —N(R^{X})—;
R^{X} is hydrogen or C₁₋₆ alkyl;
subscript m is 0, 1, 2, or 3;
subscript n is 0, 1, or 2, provided that when subscript n is 0, then X is —CH₂— or —CH(CN)—, and if subscript n is 1 or 2 then X is —O—, —CH₂—, or —N(R^{X})—;
subscript p is 0, 1, or 2;
subscript q is 0, 1, or 2; and
R⁷, R⁸, and R⁹ are each independently hydrogen or fluorine.

3. The compound of claim 2, or a pharmaceutically acceptable salt thereof, having the structure of Formula Ia:

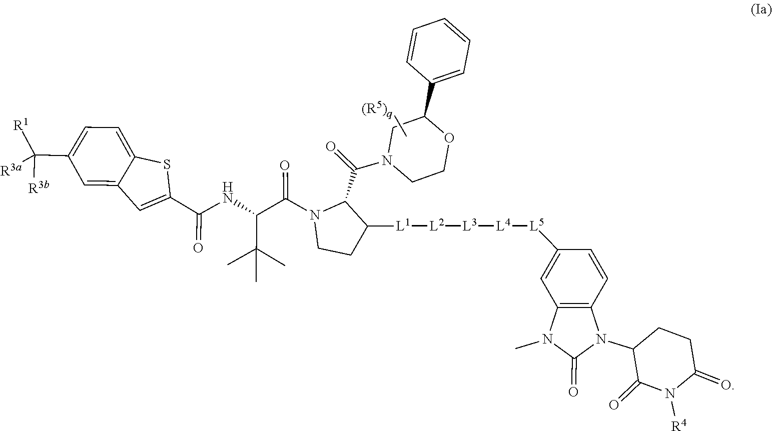

4. The compound of claim 3, or a pharmaceutically acceptable salt thereof, wherein R¹ is

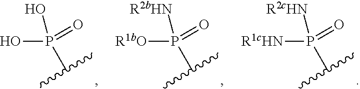

5. The compound of claim 4, or a pharmaceutically acceptable salt thereof, wherein R¹ is

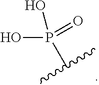 [SMILES: *P(=O)(O)O]

6. The compound of claim 4, or a pharmaceutically acceptable salt thereof, wherein R¹ is

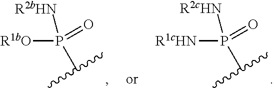

7. The compound of claim 6, or a pharmaceutically acceptable salt thereof, wherein
R^{1b} is C₆₋₁₂ aryl; and
R^{1c}, R^{2b}, and R^{2c} are each independently 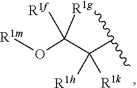
wherein
R^{1f} and R^{1g} together are oxo,
R^{1h} and R^{1k}, are each independently H or C₁₋₆ alkyl, and
R^{1m} is C₁₋₆ alkyl.

8. The compound of claim 7, or a pharmaceutically acceptable salt thereof, wherein R¹ is

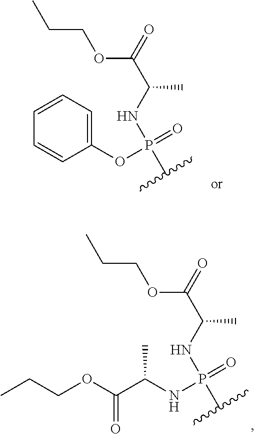

9. The compound of claim 8, or a pharmaceutically acceptable salt thereof, wherein
R^{3a} is hydrogen or F; and
R^{3b} is F.

10. The compound of claim 9, or a pharmaceutically acceptable salt thereof, wherein R^{3a} and R^{3b} are each fluoro.

11. The compound of claim 1, or a pharmaceutically acceptable salt thereof, wherein L¹ is absent, O, or NH.

12. The compound of claim 11, or a pharmaceutically acceptable salt thereof, wherein L¹ is O.

13. The compound of claim 1, or a pharmaceutically acceptable salt thereof, wherein L² is absent, CH₂, (CH₂)₂, (CH₂)₅, CH₂C(O), CH₂C(O)NHCH₂ or CH₂C(O)N(CH₃)CH₂.

14. The compound of claim 13, or a pharmaceutically acceptable salt thereof, wherein L² is CH₂ or CH₂C(O).

15. The compound of claim 14, or a pharmaceutically acceptable salt thereof, wherein L² is CH₂C(O).

16. The compound of claim 1, or a pharmaceutically acceptable salt thereof, wherein L³ is absent,

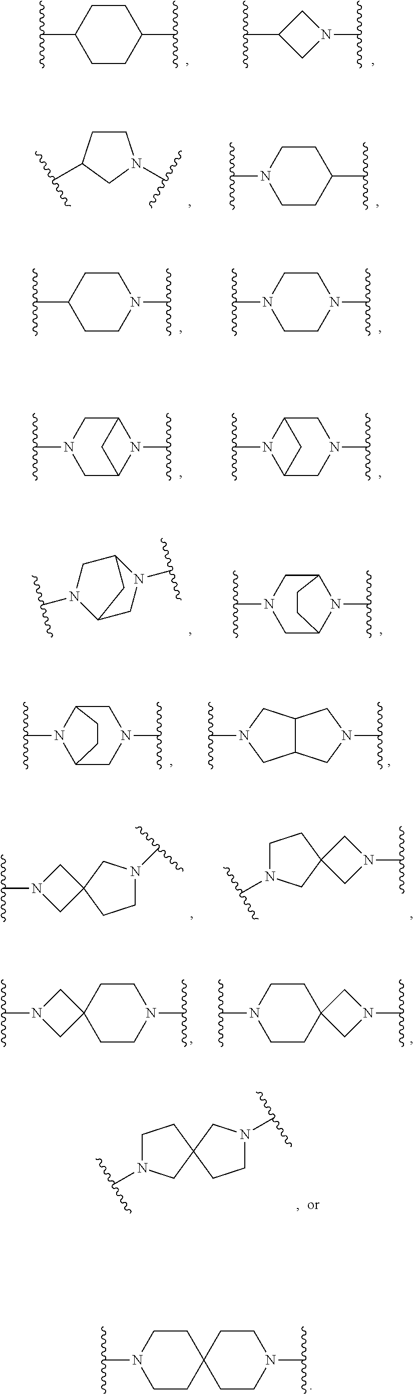

17. The compound of claim 16, or a pharmaceutically acceptable salt thereof, wherein L³ is

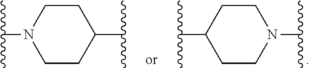 [SMILES: *C1CCN(*)CC1 ; *C1CCN(*)CC1]

18. The compound of claim 1, or a pharmaceutically acceptable salt thereof, wherein L⁴ is absent, CH₂, CH₂CH₂, —C≡C—, O, or C(O).

19. The compound claim 18, or a pharmaceutically acceptable salt thereof, wherein L⁴ is CH₂.

20. The compound of claim 19, or a pharmaceutically acceptable salt thereof, wherein L⁵ is absent,

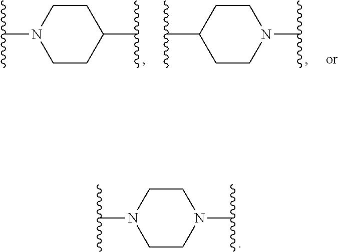

21. The compound of claim 1, or a pharmaceutically acceptable salt thereof, wherein L⁵ is absent.

22. The compound of claim 1, or a pharmaceutically acceptable salt thereof, wherein at least two of L¹, L³ and L⁴ are not absent.

23. The compound of claim 1, or a pharmaceutically acceptable salt thereof, wherein L is

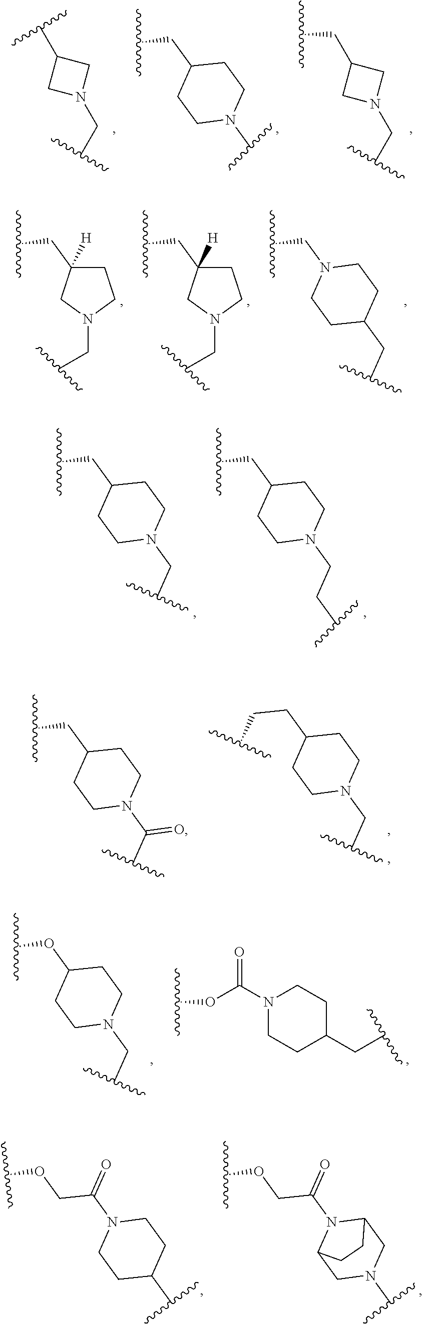

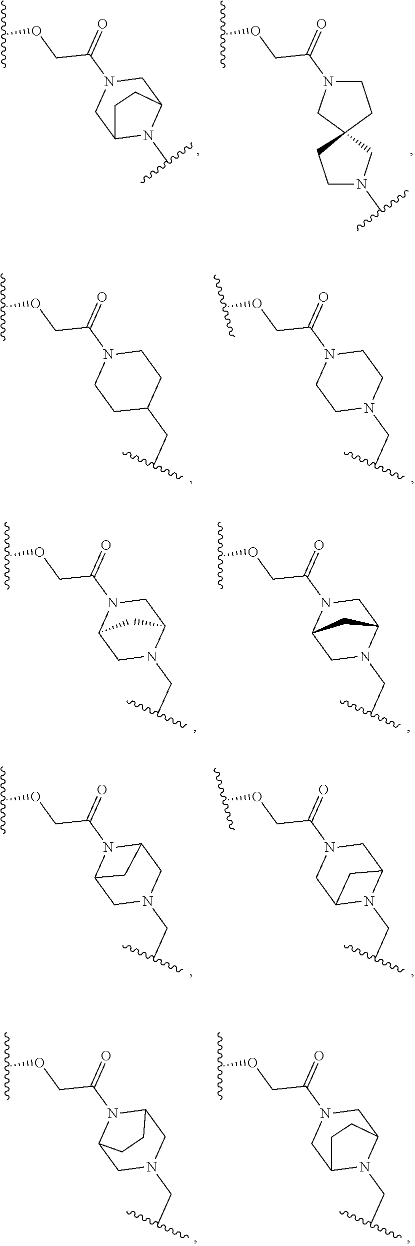

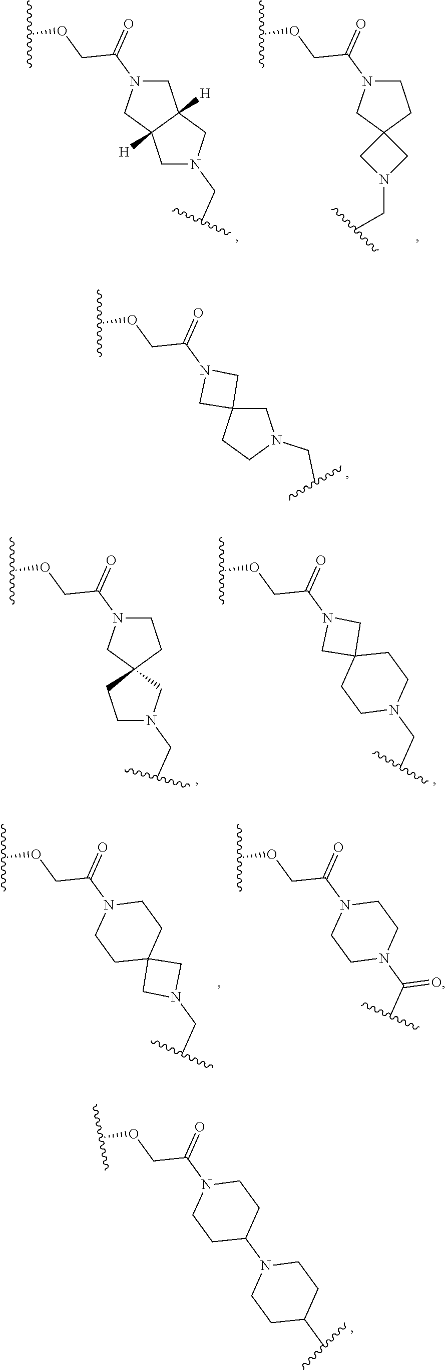

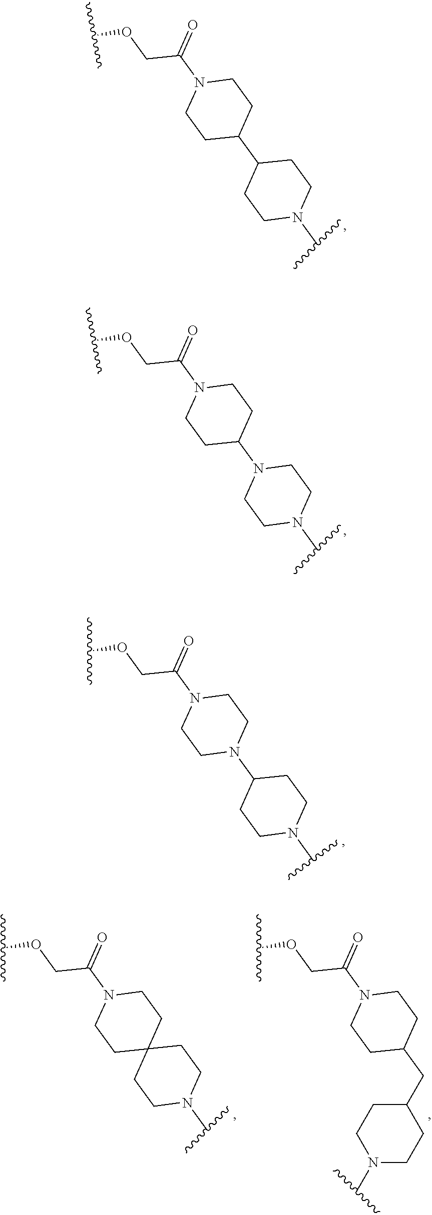

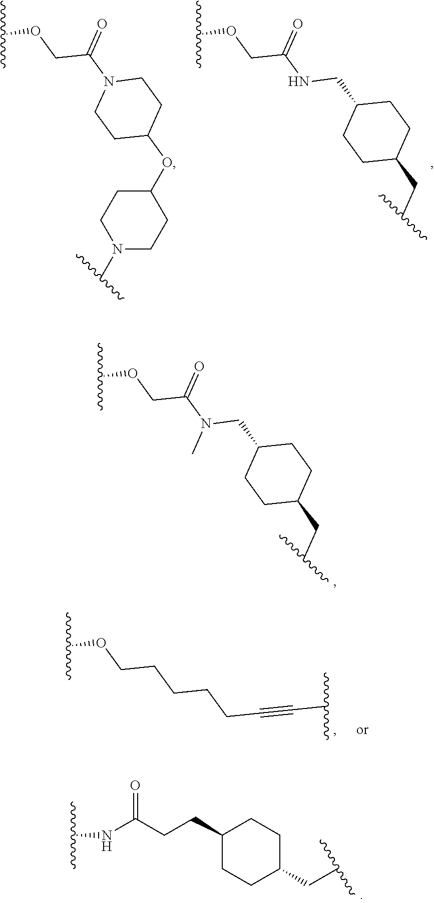

24. The compound of claim 23, or a pharmaceutically acceptable salt thereof, wherein L is:

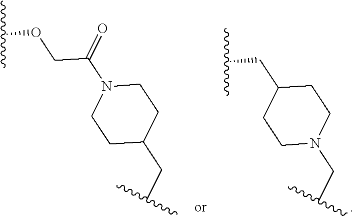

25. The compound of claim 1, or a pharmaceutically acceptable salt thereof, wherein R⁴ is hydrogen.

26. The compound of claim 1, or a pharmaceutically acceptable salt thereof, wherein
each R⁵ is hydrogen, or each R⁵ is F;
alternatively, where two R⁵ groups are bound to the same ring atom, such R⁵ groups can be taken together with the atom to which they are attached to form a C₃₋₅ cycloalkyl;
R⁶ is phenyl or pyridyl;
X is —CH₂—, —CH(CN)—, —CF₂—, —O—, or —N(CH₃)—;
subscript n is 0 or 1, provided that when subscript n is 0, then X is —CH₂— or —CH(CN)—, and if subscript n is 1 then X is —CH₂—, —CF₂—, —O—, or —N(CH₃)—; and
subscript q is 0 or 2.

27. The compound of claim 1, or a pharmaceutically acceptable salt thereof, wherein
the moiety 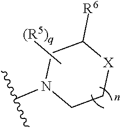
has the structure

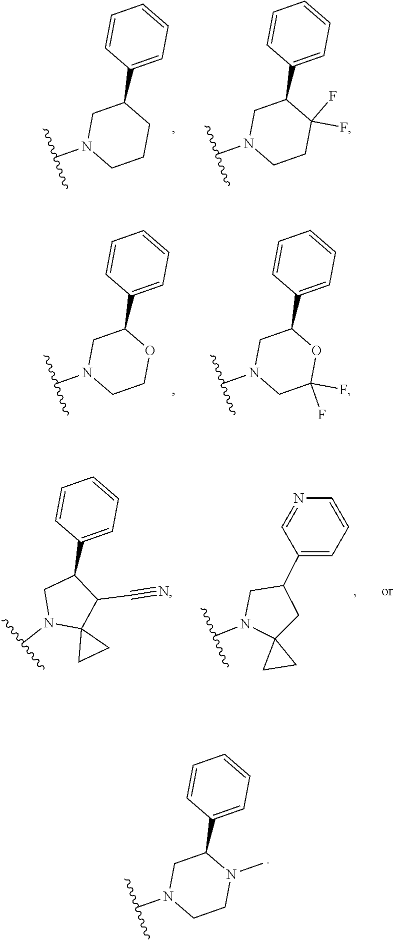

28. The compound of claim 27, or a pharmaceutically acceptable salt thereof, wherein
the moiety 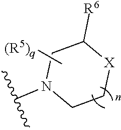
has the structure

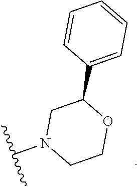 [SMILES: *N1CCO[C@H](c2ccccc2)C1]

29. The compound of claim 1, or a pharmaceutically acceptable salt thereof, wherein R⁷, R⁸, and R⁹ are each hydrogen or F.

30. The compound of claim 1, or a pharmaceutically acceptable salt thereof, having the structure of:

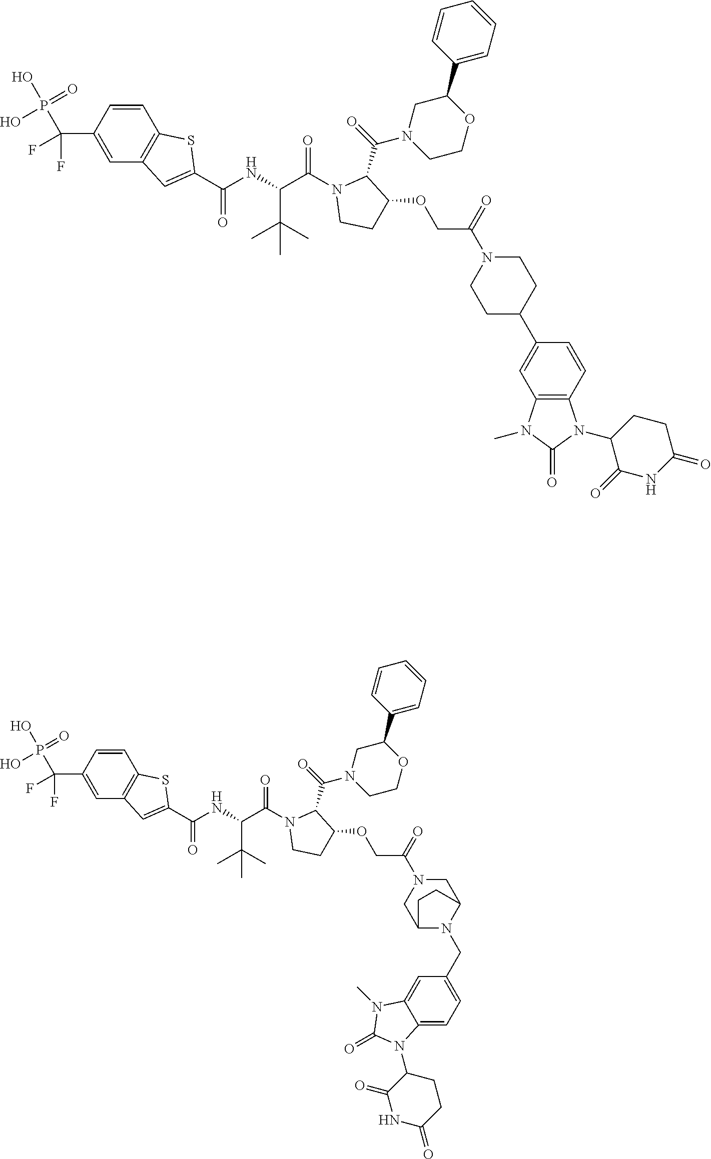 [SMILES: Cn1c(=O)n(C2CCC(=O)NC2=O)c2ccc(C3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(CN3C4CCC3CN(C(=O)CO[C@@H]3CCN(C(=O)[C@@H](NC(=O)c5cc6cc(C(F)(F)P(=O)(O)O)ccc6s5)C(C)(C)C)[C@@H]3C(=O)N3CCO[C@H](c5ccccc5)C3)C4)cc21]

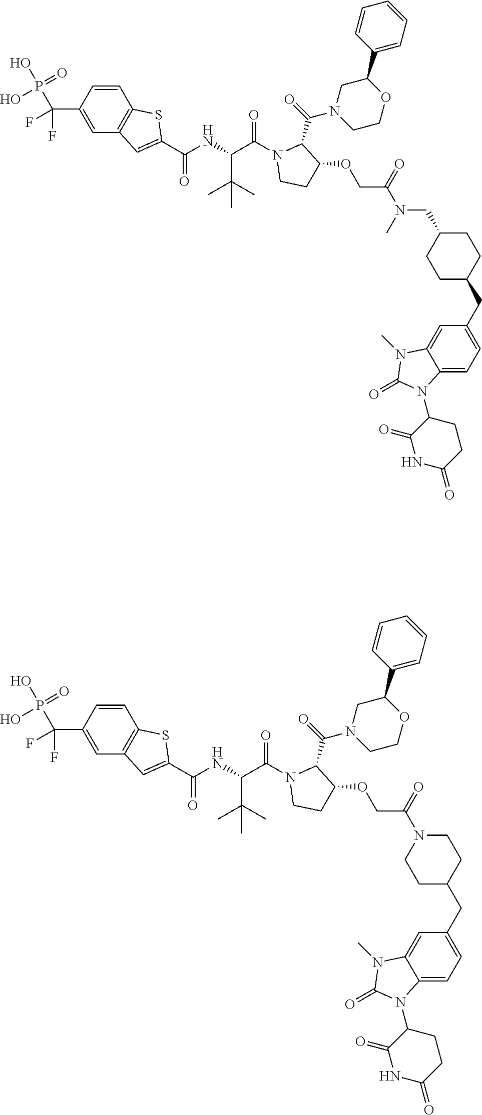 [SMILES: CN(C[C@H]1CC[C@H](Cc2ccc3c(c2)n(C)c(=O)n3C2CCC(=O)NC2=O)CC1)C(=O)CO[C@@H]1CCN(C(=O)[C@@H](NC(=O)c2cc3cc(C(F)(F)P(=O)(O)O)ccc3s2)C(C)(C)C)[C@@H]1C(=O)N1CCO[C@H](c2ccccc2)C1 ; Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21]

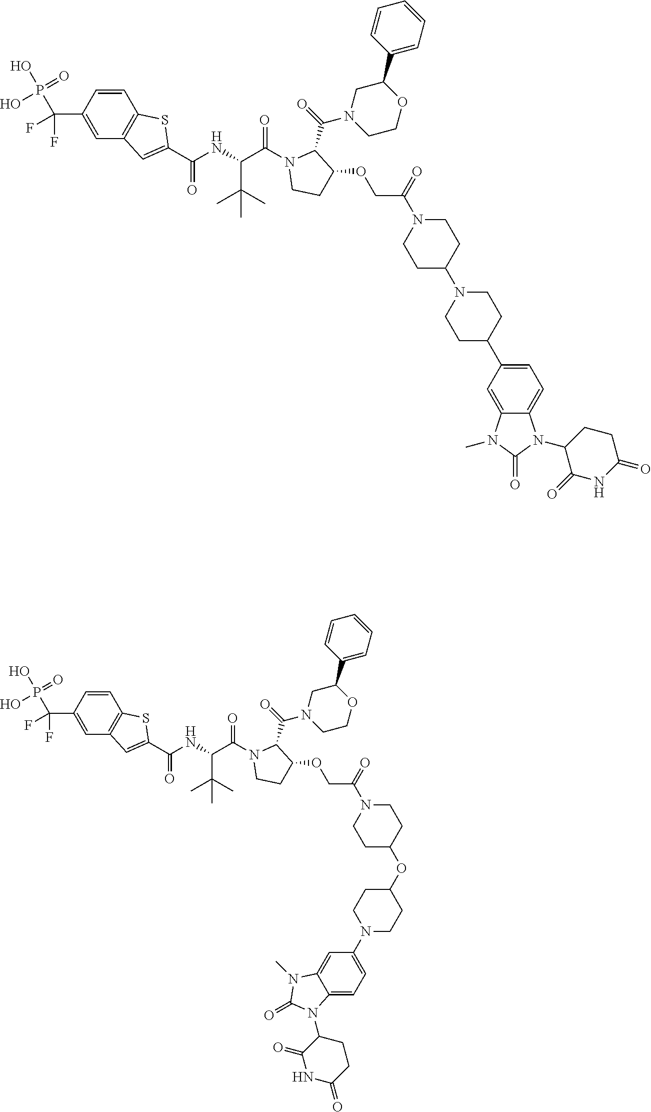 [SMILES: Cn1c(=O)n(C2CCC(=O)NC2=O)c2ccc(C3CCN(C4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21 ; Cn1c(=O)n(C2CCC(=O)NC2=O)c2ccc(N3CCC(OC4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21]

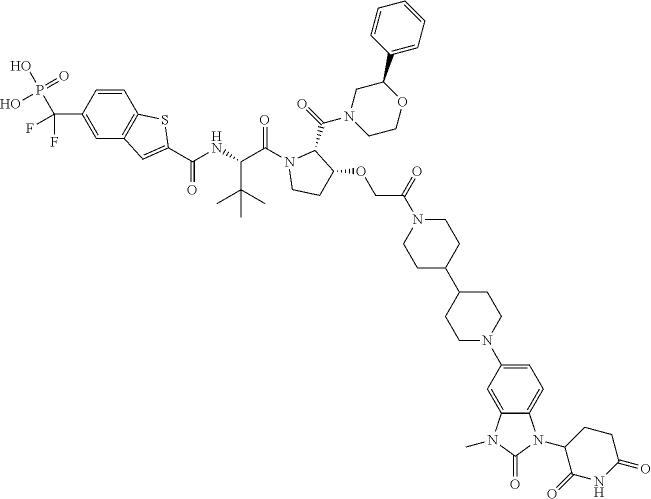 [SMILES: Cn1c(=O)n(C2CCC(=O)NC2=O)c2ccc(N3CCC(C4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21]

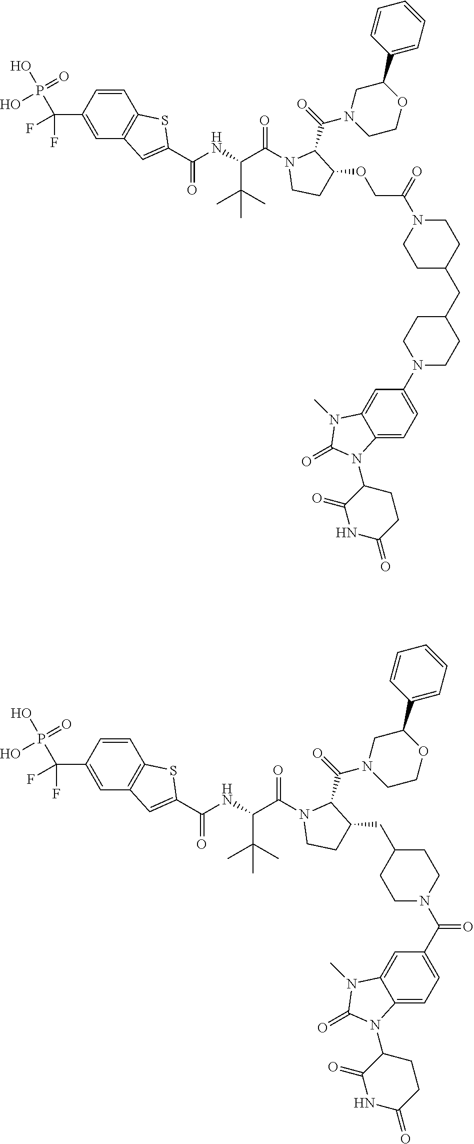 [SMILES: Cn1c(=O)n(C2CCC(=O)NC2=O)c2ccc(N3CCC(CC4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21 ; Cn1c(=O)n(C2CCC(=O)NC2=O)c2ccc(C(=O)N3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21]

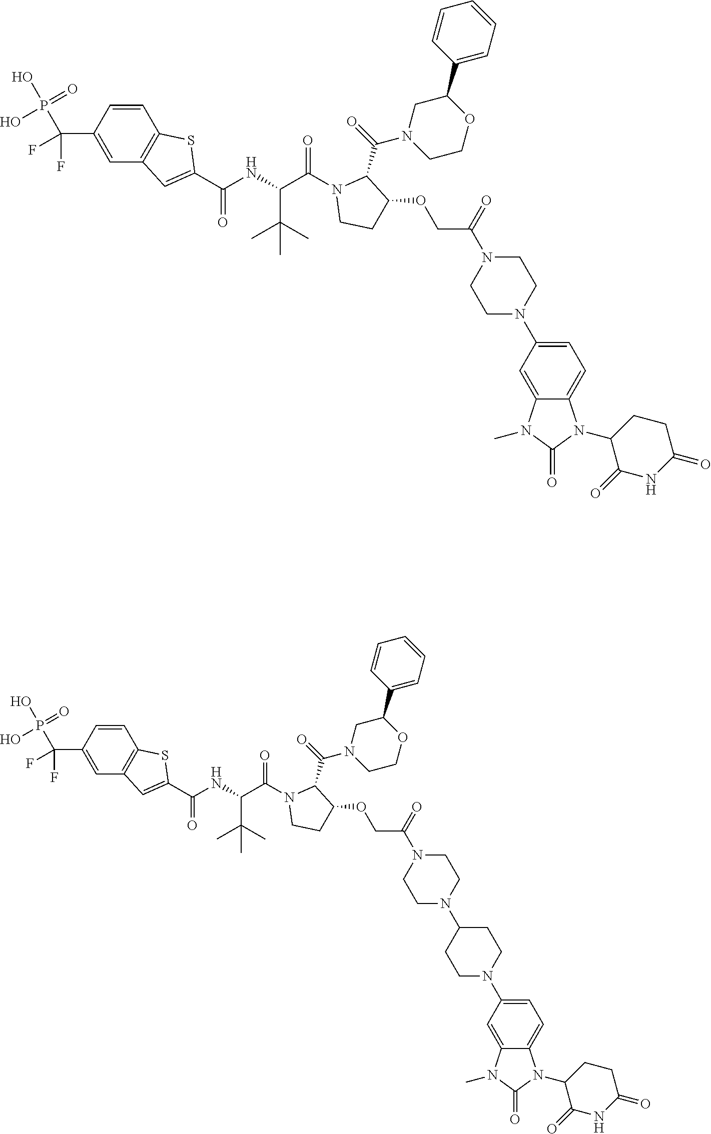 [SMILES: Cn1c(=O)n(C2CCC(=O)NC2=O)c2ccc(N3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(N3CCC(N4CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21]

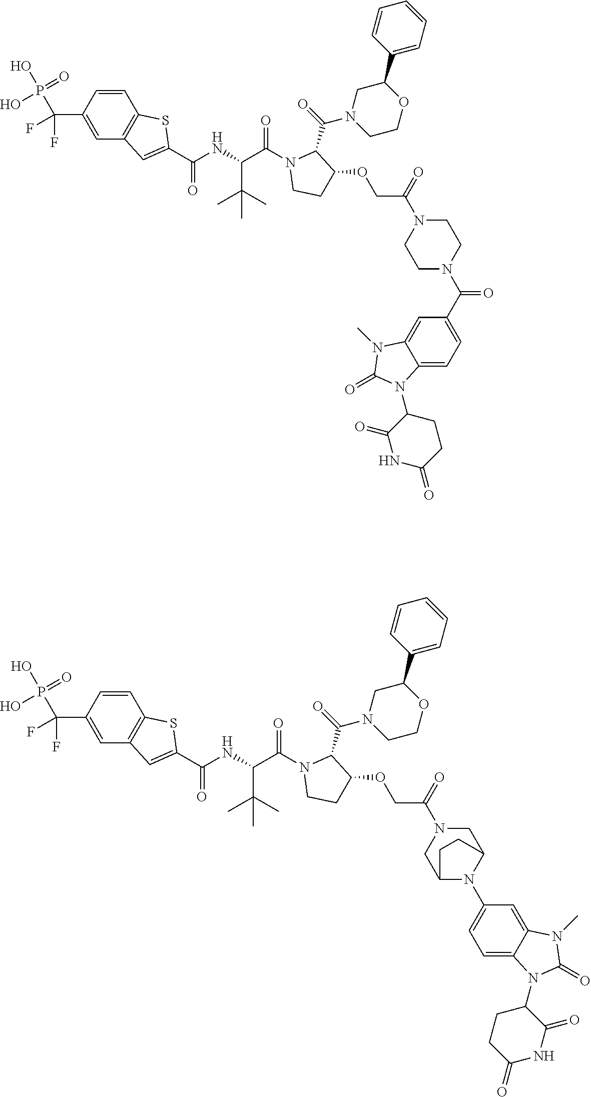 [SMILES: Cn1c(=O)n(C2CCC(=O)NC2=O)c2ccc(C(=O)N3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(N3C4CCC3CN(C(=O)CO[C@@H]3CCN(C(=O)[C@@H](NC(=O)c5cc6cc(C(F)(F)P(=O)(O)O)ccc6s5)C(C)(C)C)[C@@H]3C(=O)N3CCO[C@H](c5ccccc5)C3)C4)cc21]

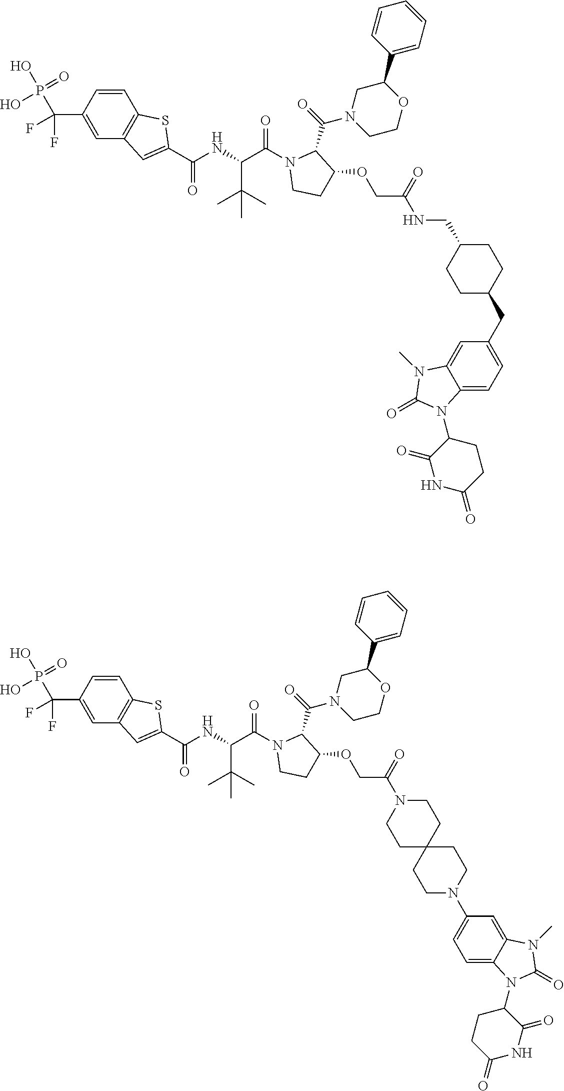 [SMILES: Cn1c(=O)n(C2CCC(=O)NC2=O)c2ccc(C[C@H]3CC[C@H](CNC(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(N3CCC4(CCN(C(=O)CO[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)CC3)cc21]

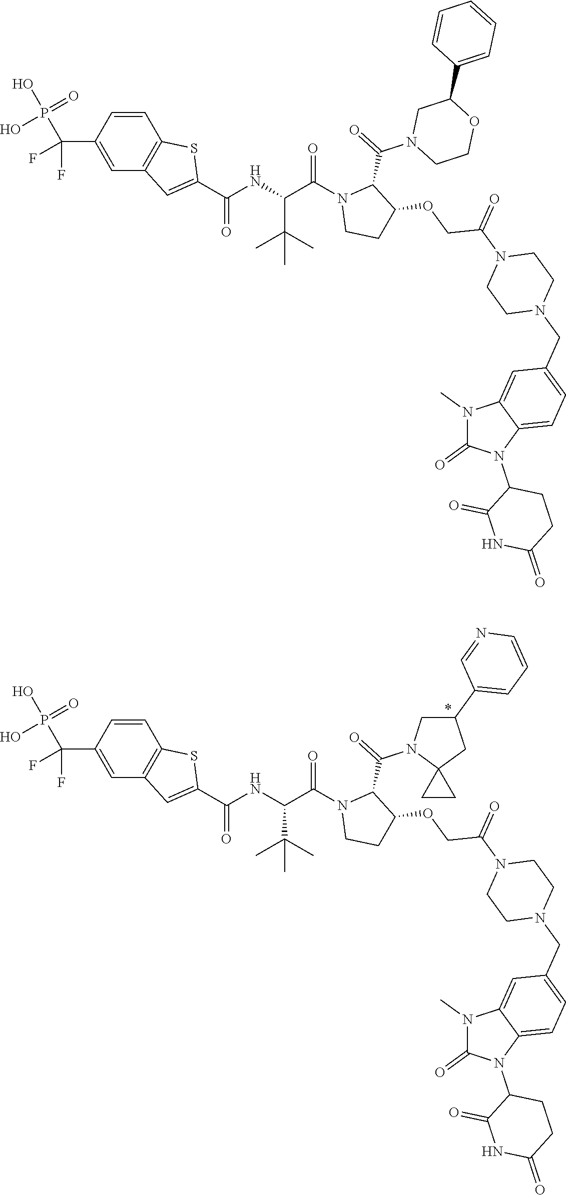 [SMILES: Cn1c(=O)n(C2CCC(=O)NC2=O)c2ccc(CN3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(CN3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CC(c5cccnc5)CC45CC5)CC3)cc21]

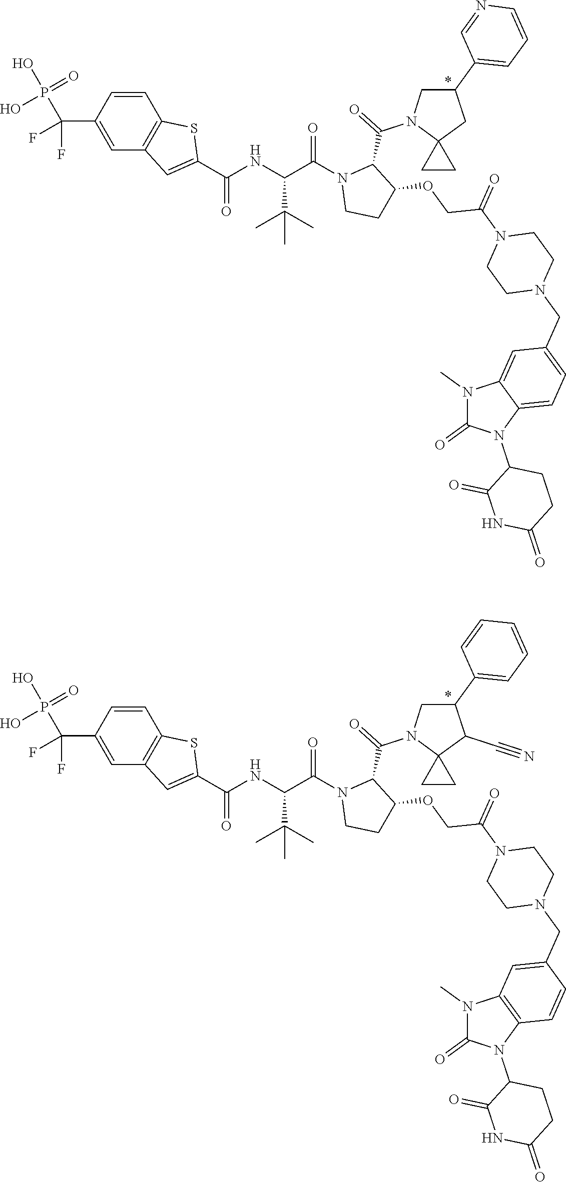 [SMILES: Cn1c(=O)n(C2CCC(=O)NC2=O)c2ccc(CN3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CC(c5cccnc5)CC45CC5)CC3)cc21 ; Cn1c(=O)n(C2CCC(=O)NC2=O)c2ccc(CN3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CC(c5ccccc5)C(C#N)C45CC5)CC3)cc21]

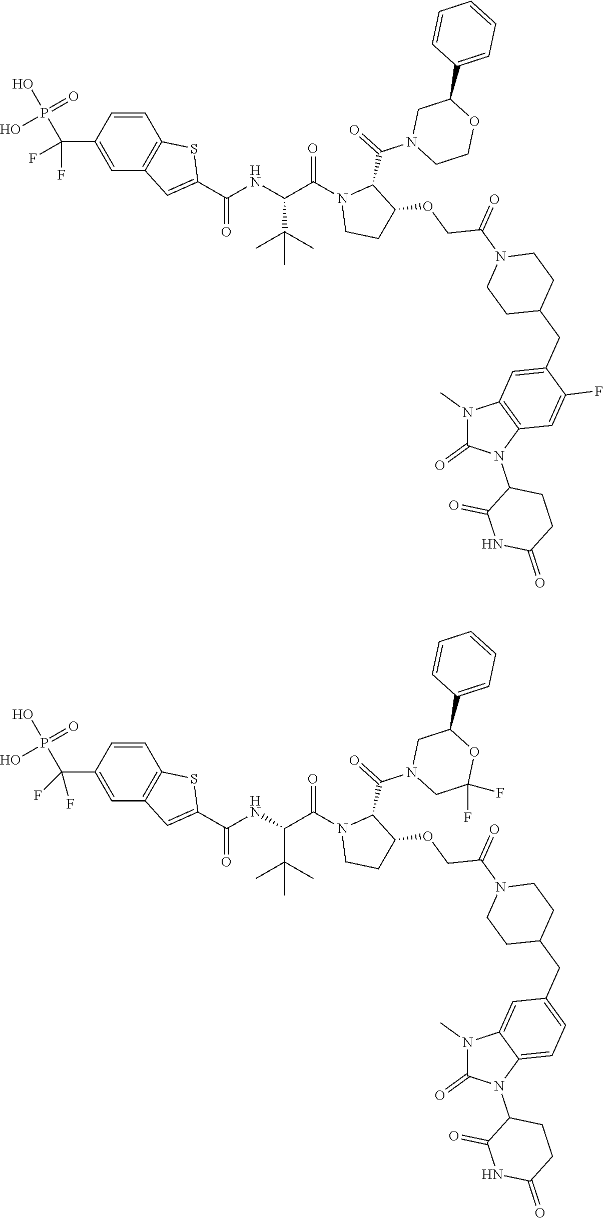 [SMILES: Cn1c(=O)n(C2CCC(=O)NC2=O)c2cc(F)c(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4C[C@@H](c5ccccc5)OC(F)(F)C4)CC3)cc21]

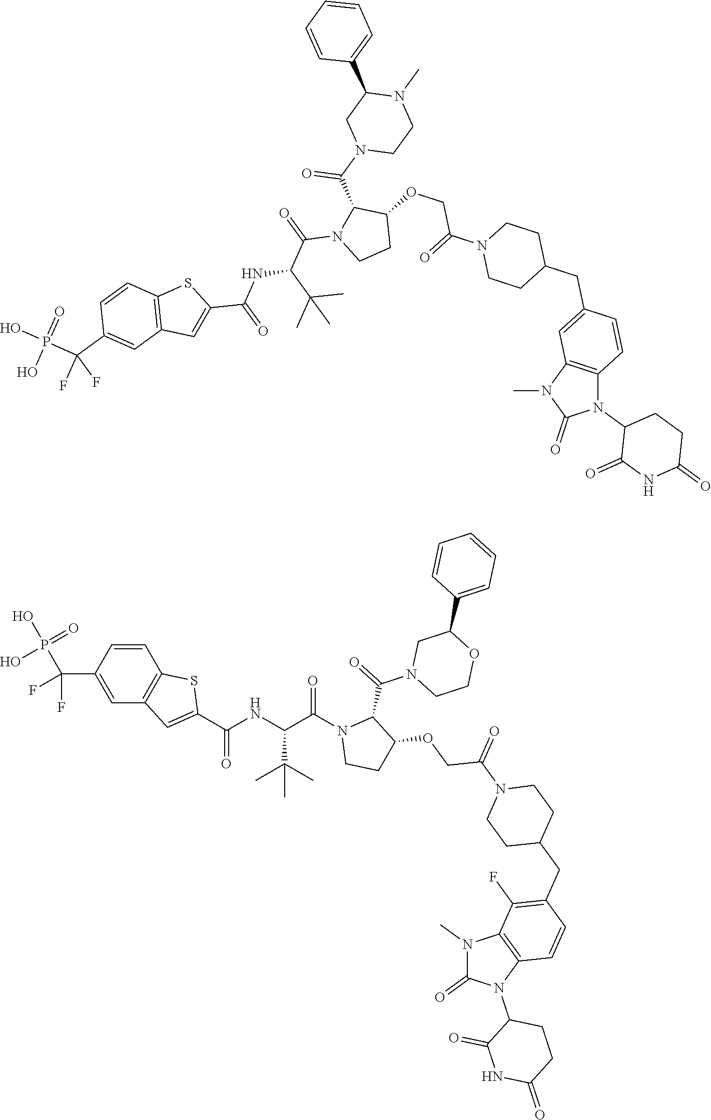 [SMILES: CN1CCN(C(=O)[C@@H]2[C@H](OCC(=O)N3CCC(Cc4ccc5c(c4)n(C)c(=O)n5C4CCC(=O)NC4=O)CC3)CCN2C(=O)[C@@H](NC(=O)c2cc3cc(C(F)(F)P(=O)(O)O)ccc3s2)C(C)(C)C)C[C@H]1c1ccccc1 ; Cn1c(=O)n(C2CCC(=O)NC2=O)c2ccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)c(F)c21]

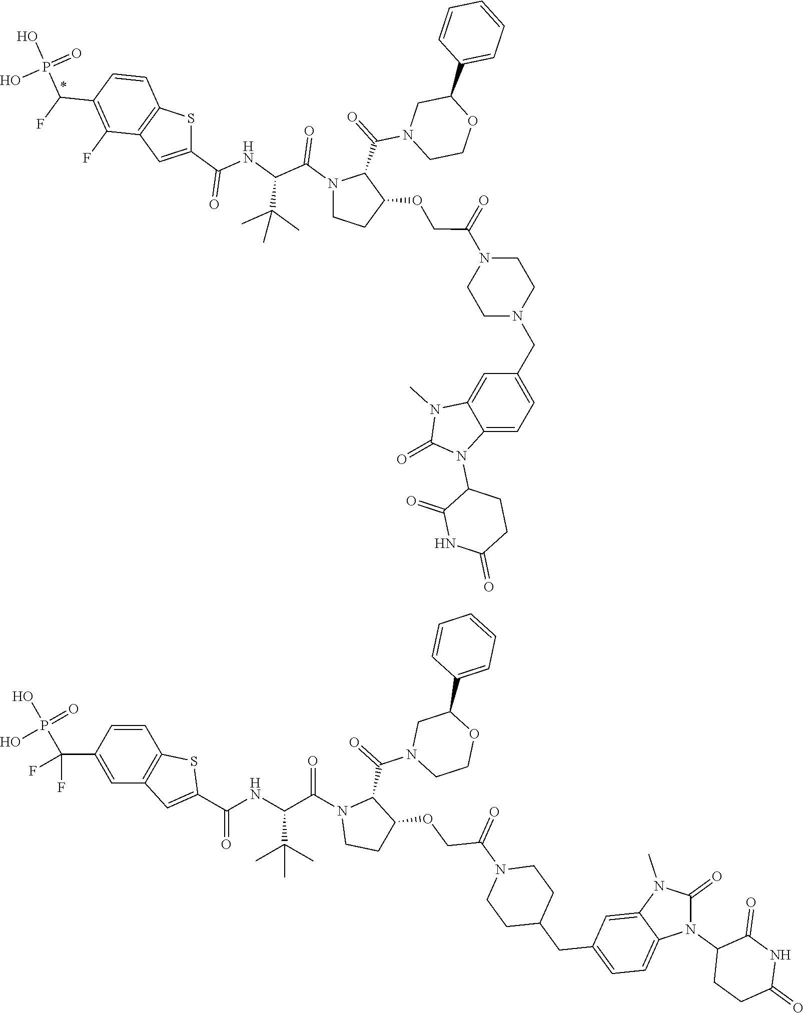

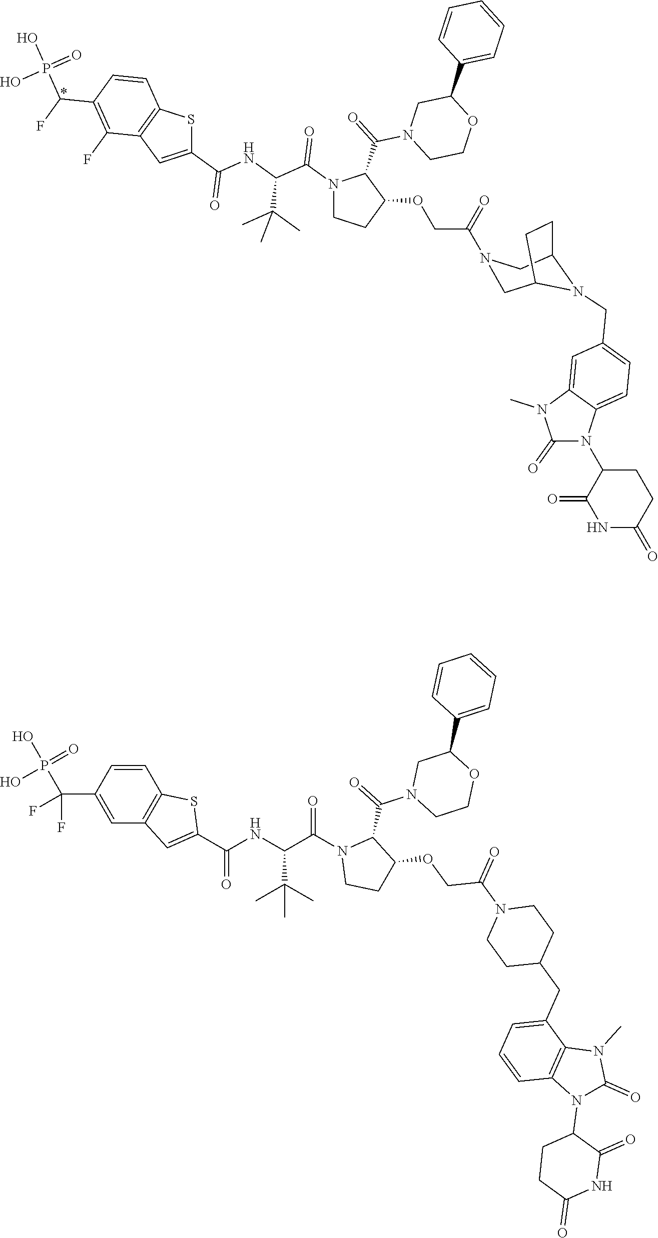 [SMILES: Cn1c(=O)n(C2CCC(=O)NC2=O)c2ccc(CN3C4CCC3CN(C(=O)CO[C@@H]3CCN(C(=O)[C@@H](NC(=O)c5cc6c(F)c(C(F)P(=O)(O)O)ccc6s5)C(C)(C)C)[C@@H]3C(=O)N3CCO[C@H](c5ccccc5)C3)C4)cc21 ; Cn1c(=O)n(C2CCC(=O)NC2=O)c2cccc(CC3CCN(C(=O)CO[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)c21]

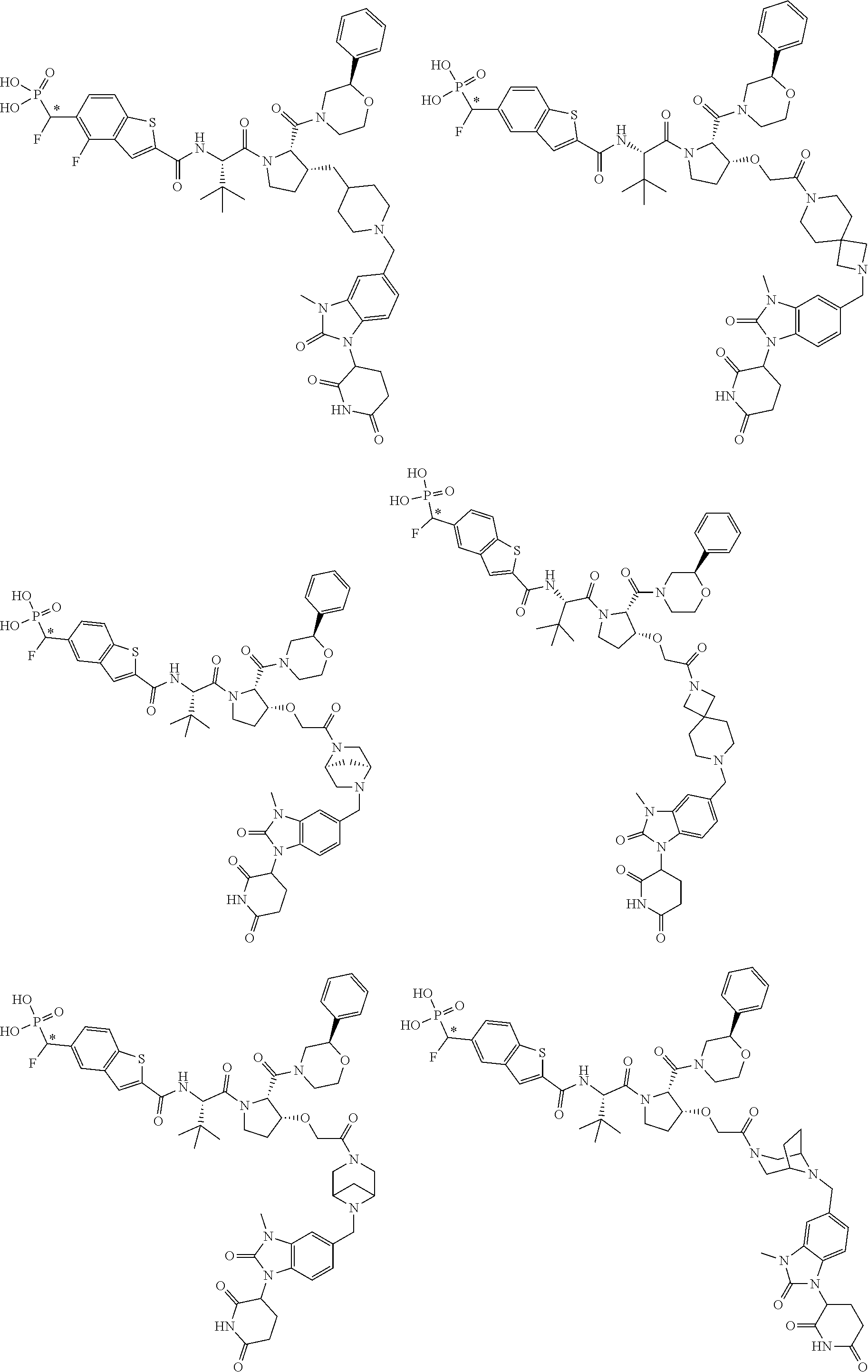

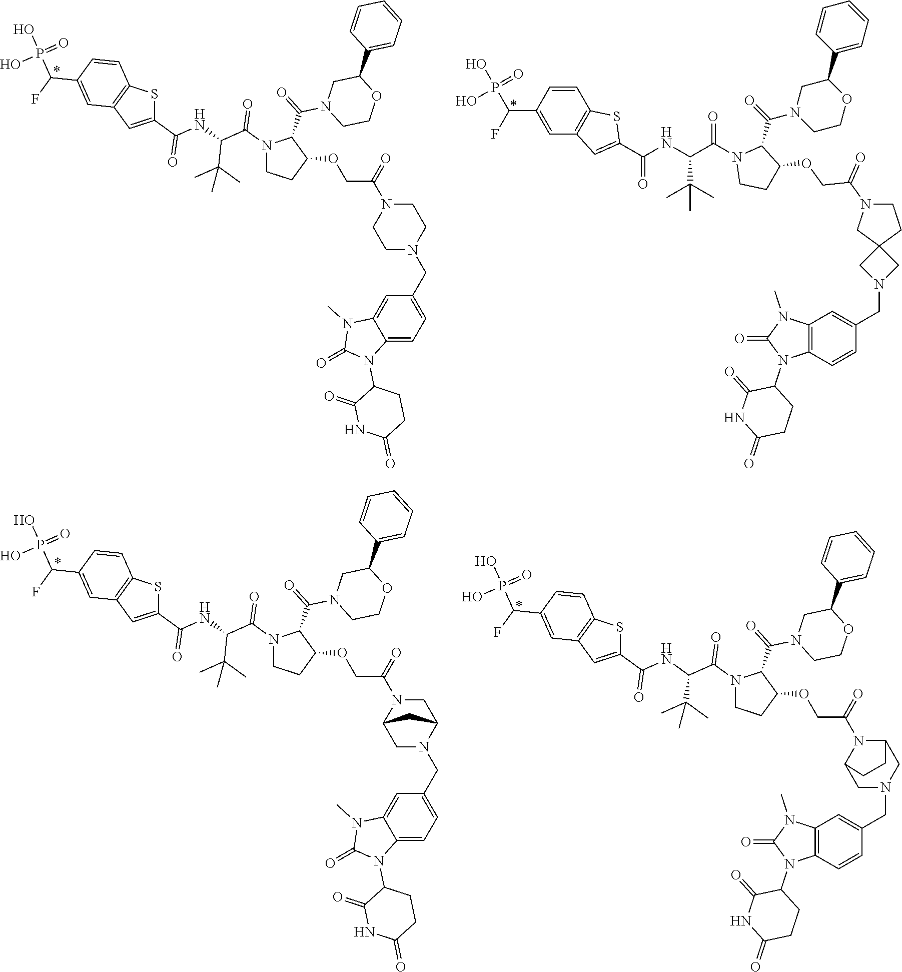

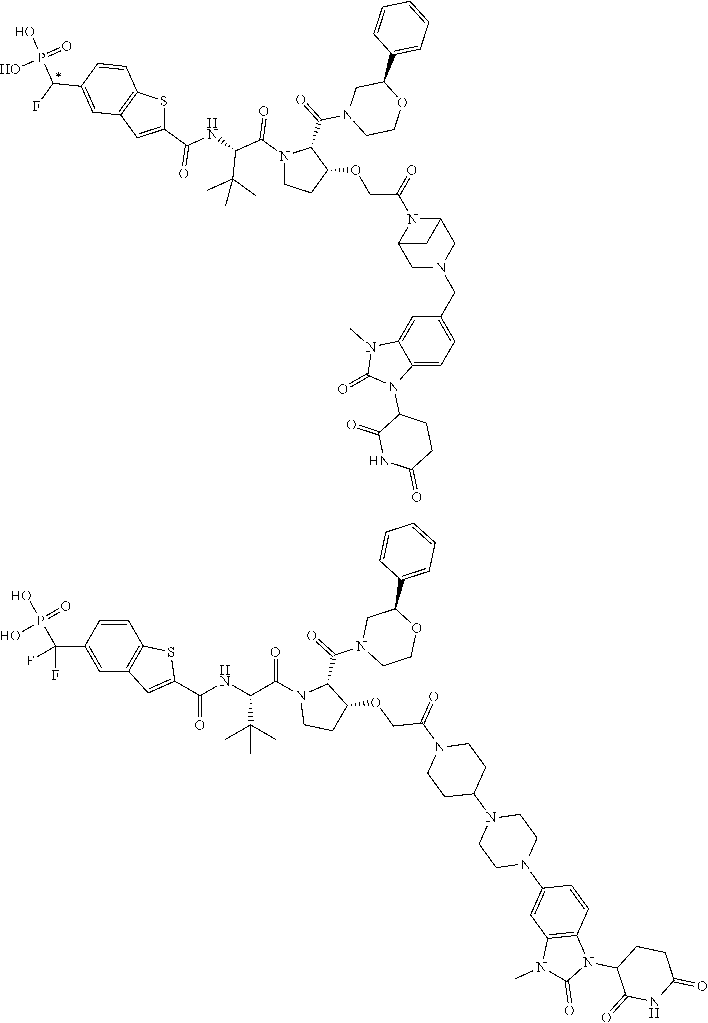

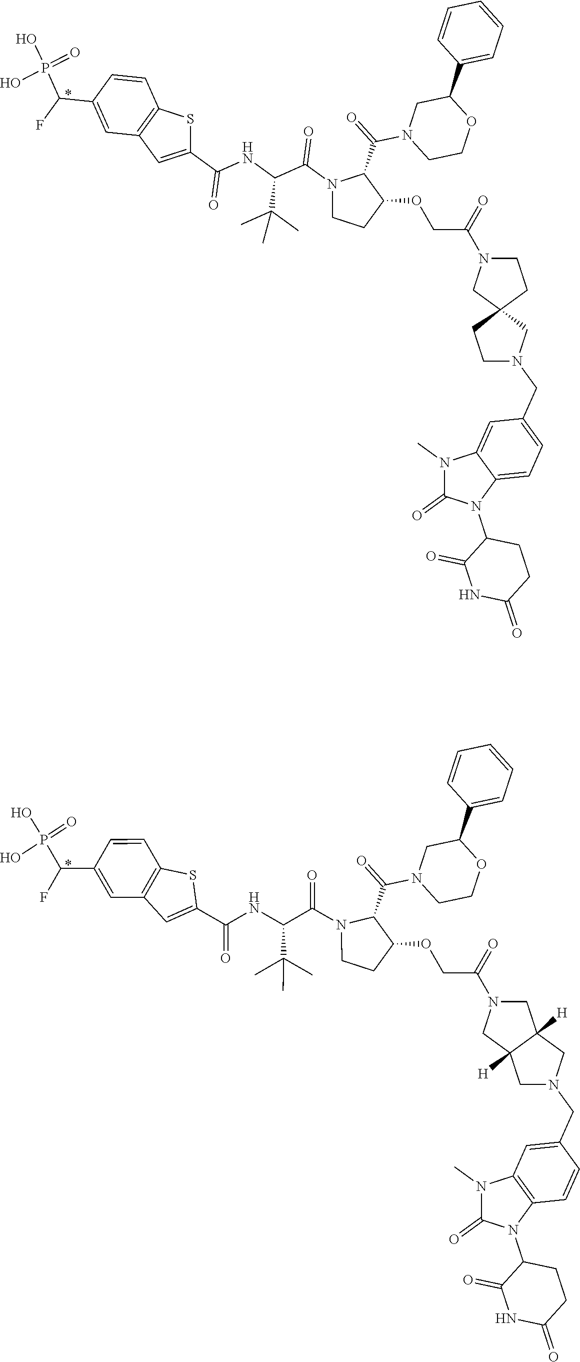 [SMILES: Cn1c(=O)n(C2CCC(=O)NC2=O)c2ccc(CN3CC[C@@]4(CCN(C(=O)CO[C@@H]5CCN(C(=O)[C@@H](NC(=O)c6cc7cc(C(F)P(=O)(O)O)ccc7s6)C(C)(C)C)[C@@H]5C(=O)N5CCO[C@H](c6ccccc6)C5)C4)C3)cc21 ; Cn1c(=O)n(C2CCC(=O)NC2=O)c2ccc(CN3C[C@@H]4CN(C(=O)CO[C@@H]5CCN(C(=O)[C@@H](NC(=O)c6cc7cc(C(F)P(=O)(O)O)ccc7s6)C(C)(C)C)[C@@H]5C(=O)N5CCO[C@H](c6ccccc6)C5)C[C@@H]4C3)cc21]

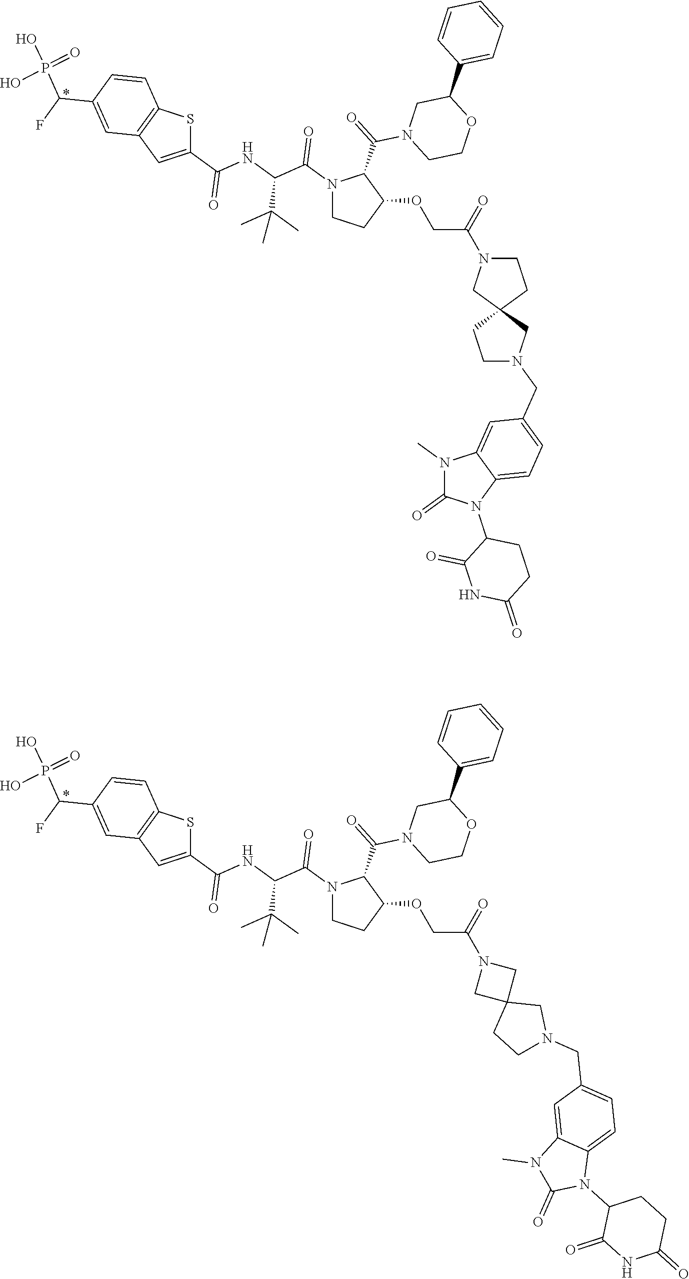 [SMILES: Cn1c(=O)n(C2CCC(=O)NC2=O)c2ccc(CN3CC[C@]4(CCN(C(=O)CO[C@@H]5CCN(C(=O)[C@@H](NC(=O)c6cc7cc(C(F)P(=O)(O)O)ccc7s6)C(C)(C)C)[C@@H]5C(=O)N5CCO[C@H](c6ccccc6)C5)C4)C3)cc21 ; Cn1c(=O)n(C2CCC(=O)NC2=O)c2ccc(CN3CCC4(C3)CN(C(=O)CO[C@@H]3CCN(C(=O)[C@@H](NC(=O)c5cc6cc(C(F)P(=O)(O)O)ccc6s5)C(C)(C)C)[C@@H]3C(=O)N3CCO[C@H](c5ccccc5)C3)C4)cc21]

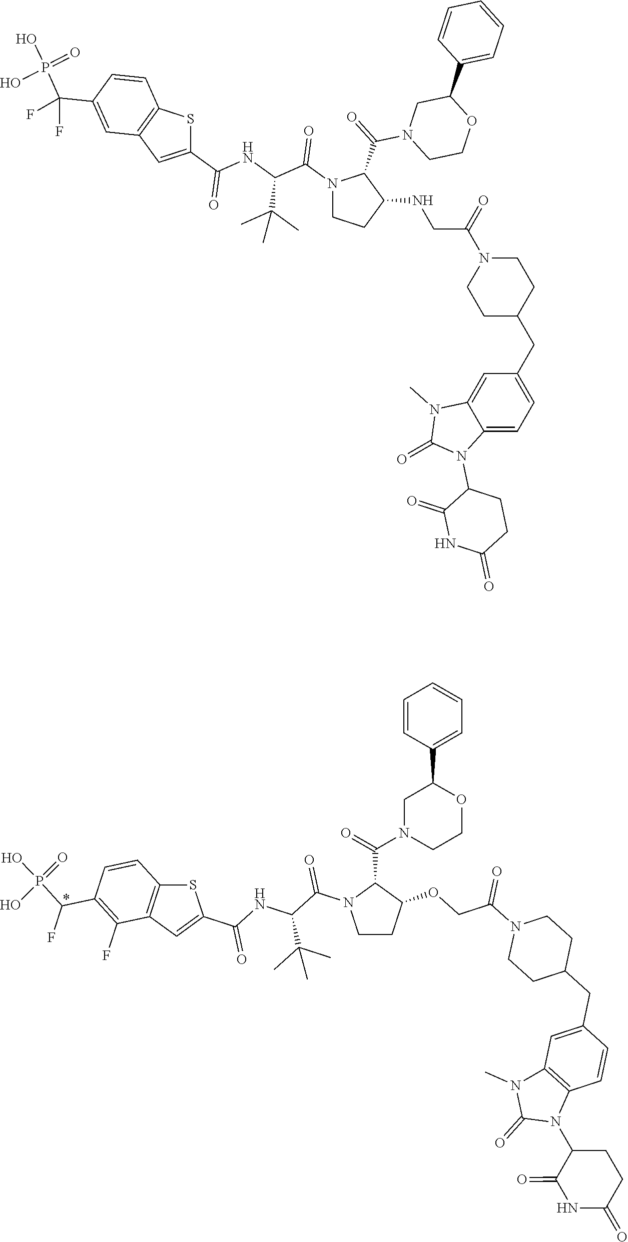 [SMILES: Cn1c(=O)n(C2CCC(=O)NC2=O)c2ccc(CC3CCN(C(=O)CN[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(CC3CCN(C(=O)CO[C@@H]4CCN(C(=O)[C@@H](NC(=O)c5cc6c(F)c(C(F)P(=O)(O)O)ccc6s5)C(C)(C)C)[C@@H]4C(=O)N4CCO[C@H](c5ccccc5)C4)CC3)cc21]

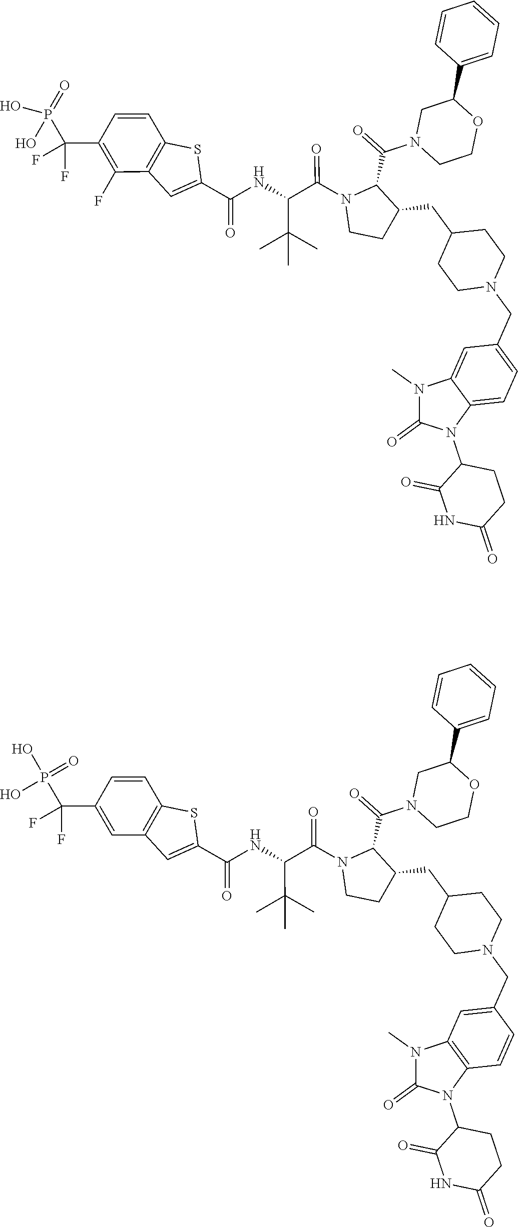 [SMILES: Cn1c(=O)n(C2CCC(=O)NC2=O)c2ccc(CN3CCC(C[C@@H]4CCN(C(=O)[C@@H](NC(=O)c5cc6c(F)c(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(CN3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21]

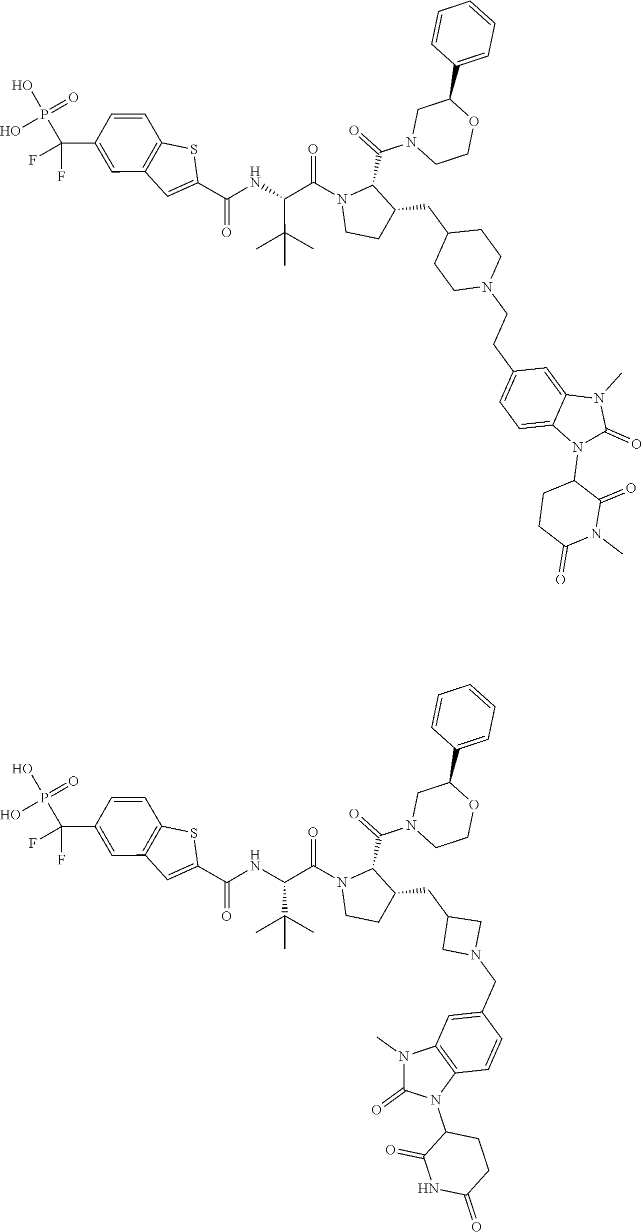 [SMILES: CN1C(=O)CCC(n2c(=O)n(C)c3cc(CCN4CCC(C[C@@H]5CCN(C(=O)[C@@H](NC(=O)c6cc7cc(C(F)(F)P(=O)(O)O)ccc7s6)C(C)(C)C)[C@@H]5C(=O)N5CCO[C@H](c6ccccc6)C5)CC4)ccc32)C1=O ; Cn1c(=O)n(C2CCC(=O)NC2=O)c2ccc(CN3CC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)C3)cc21]

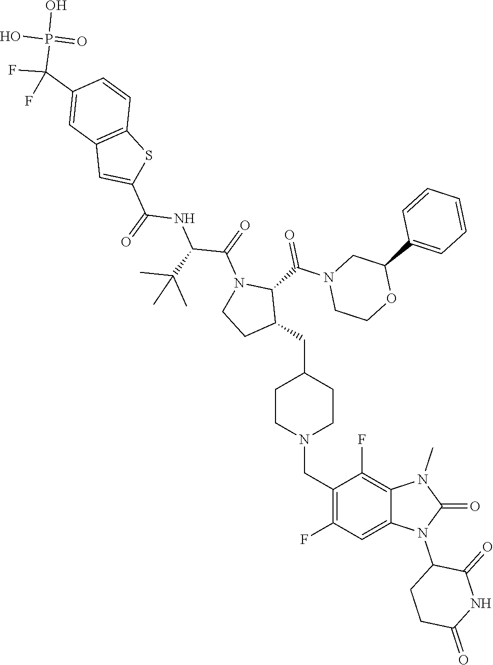 [SMILES: Cn1c(=O)n(C2CCC(=O)NC2=O)c2cc(F)c(CN3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)c(F)c21]

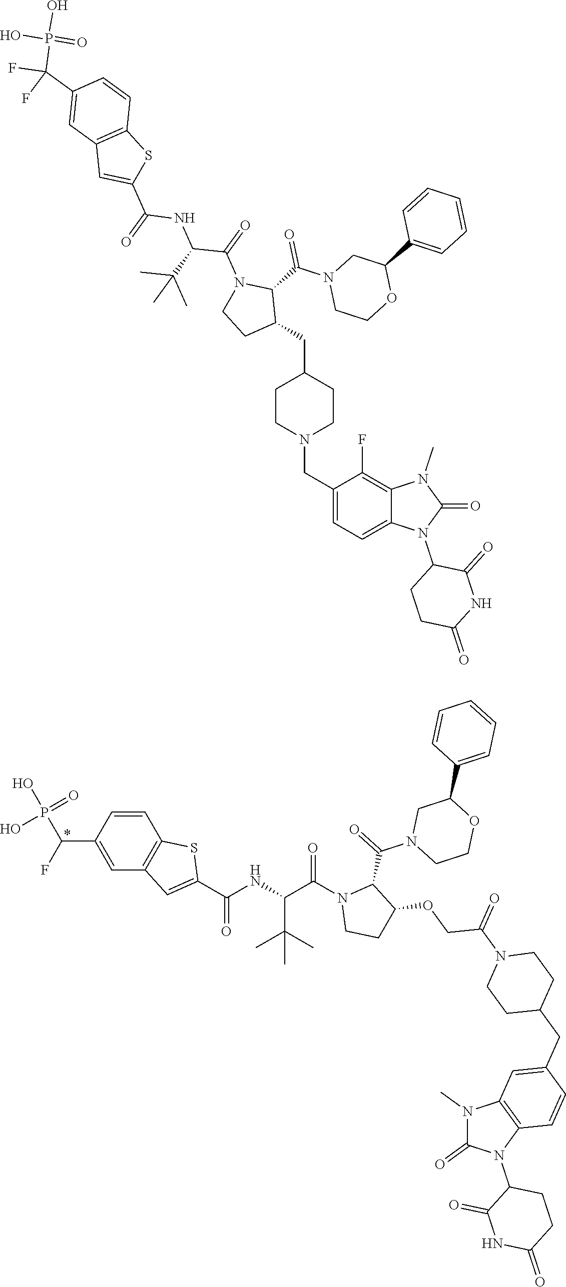

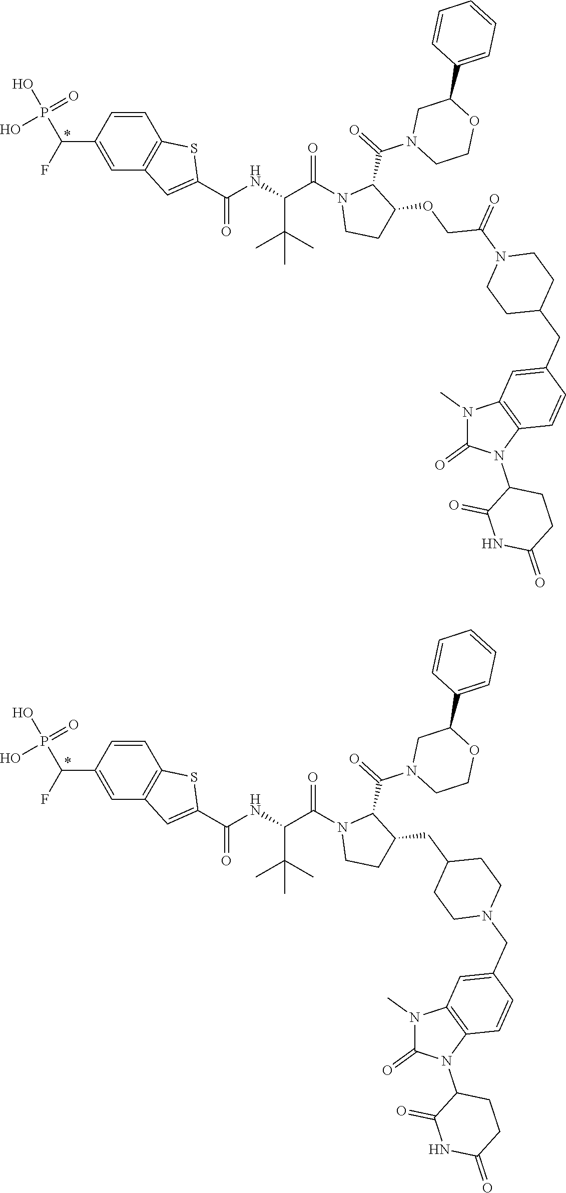 [SMILES: Cn1c(=O)n(C2CCC(=O)NC2=O)c2ccc(CC3CCN(C(=O)CO[C@@H]4CCN(C(=O)[C@@H](NC(=O)c5cc6cc(C(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(CN3CCC(C[C@@H]4CCN(C(=O)[C@@H](NC(=O)c5cc6cc(C(F)P(=O)(O)O)ccc6s5)C(C)(C)C)[C@@H]4C(=O)N4CCO[C@H](c5ccccc5)C4)CC3)cc21]

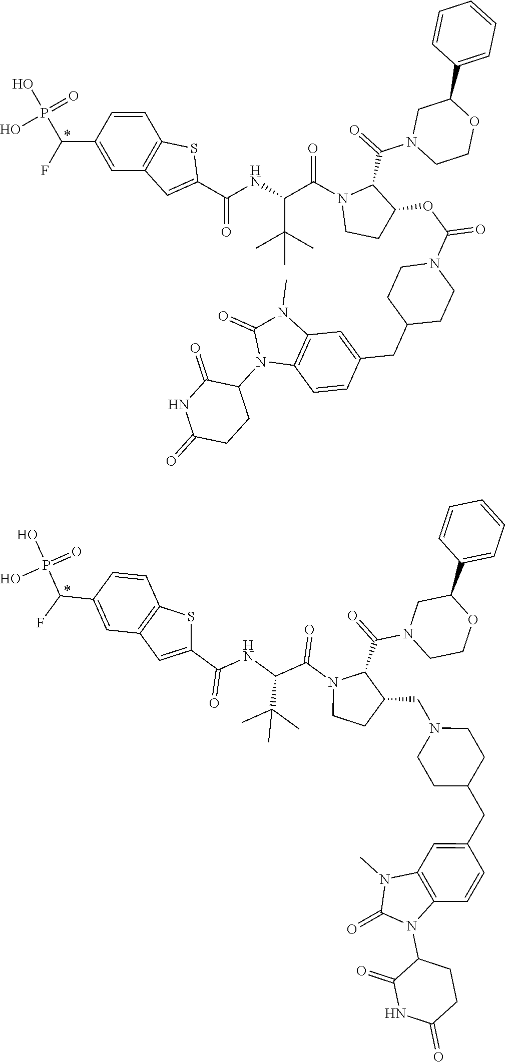 [SMILES: Cn1c(=O)n(C2CCC(=O)NC2=O)c2ccc(CC3CCN(C(=O)O[C@@H]4CCN(C(=O)[C@@H](NC(=O)c5cc6cc(C(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(CC3CCN(C[C@@H]4CCN(C(=O)[C@@H](NC(=O)c5cc6cc(C(F)P(=O)(O)O)ccc6s5)C(C)(C)C)[C@@H]4C(=O)N4CCO[C@H](c5ccccc5)C4)CC3)cc21]

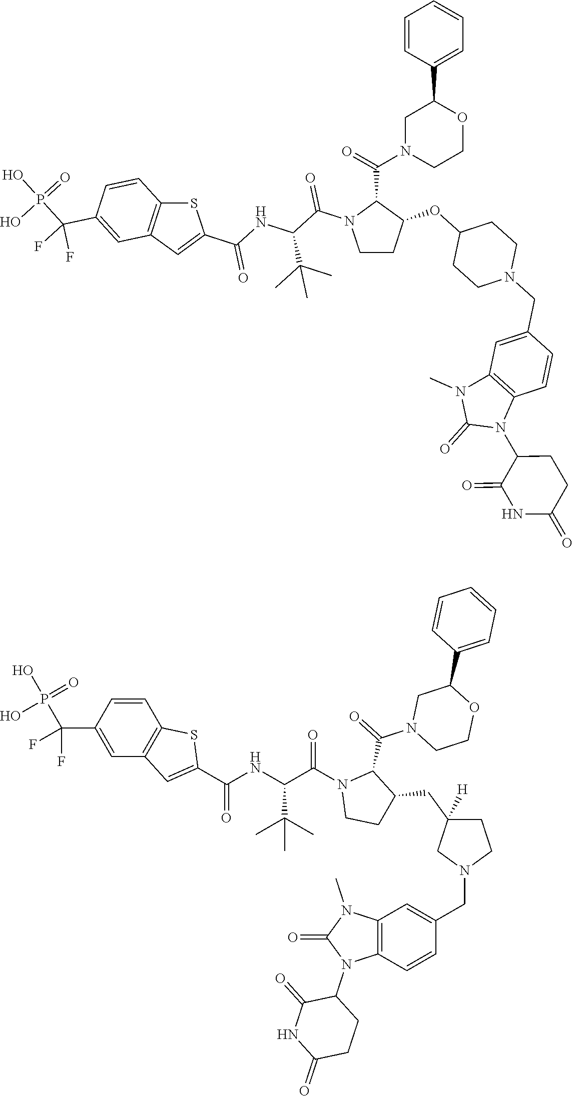 [SMILES: Cn1c(=O)n(C2CCC(=O)NC2=O)c2ccc(CN3CCC(O[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(CN3CC[C@H](C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)C3)cc21]

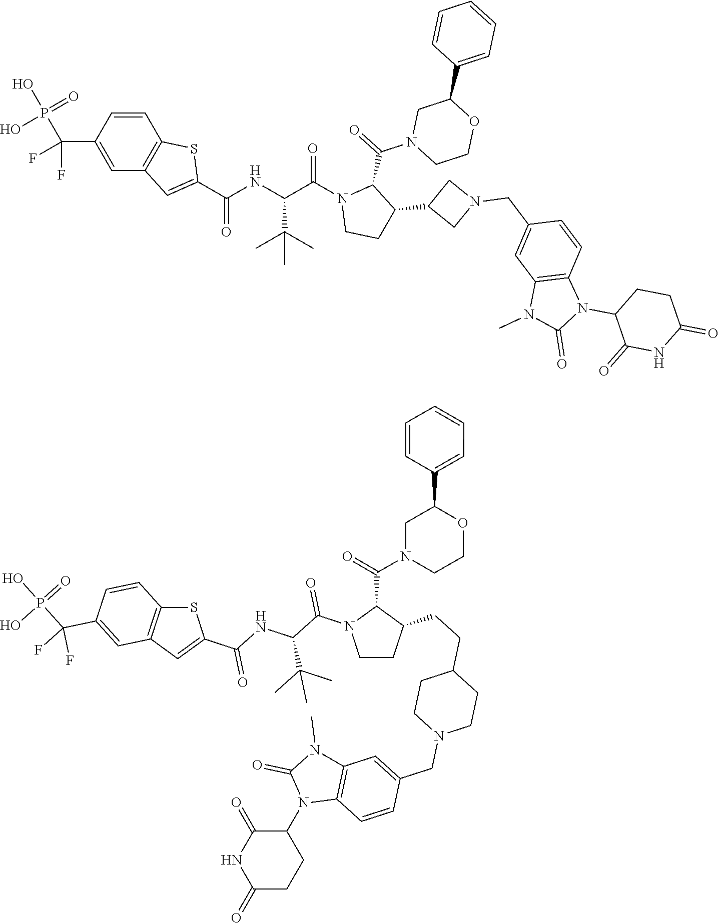 [SMILES: Cn1c(=O)n(C2CCC(=O)NC2=O)c2ccc(CN3CC([C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)C3)cc21 ; Cn1c(=O)n(C2CCC(=O)NC2=O)c2ccc(CN3CCC(CC[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21]

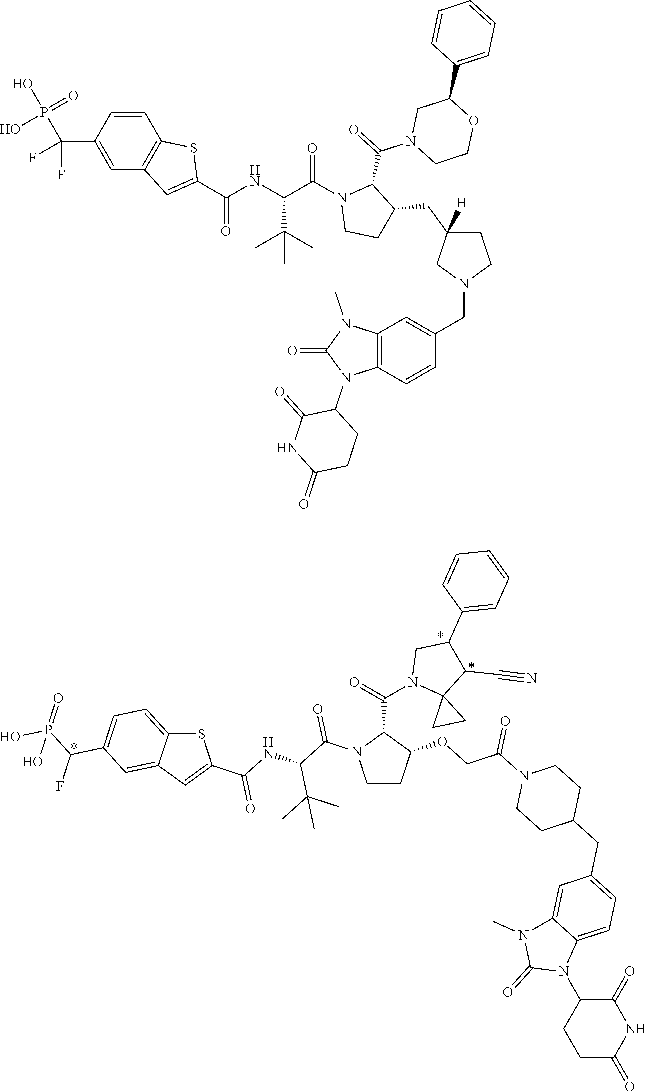 [SMILES: Cn1c(=O)n(C2CCC(=O)NC2=O)c2ccc(CN3CC[C@@H](C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)C3)cc21 ; Cn1c(=O)n(C2CCC(=O)NC2=O)c2ccc(CC3CCN(C(=O)CO[C@@H]4CCN(C(=O)[C@@H](NC(=O)c5cc6cc(C(F)P(=O)(O)O)ccc6s5)C(C)(C)C)[C@@H]4C(=O)N4CC(c5ccccc5)C(C#N)C45CC5)CC3)cc21]

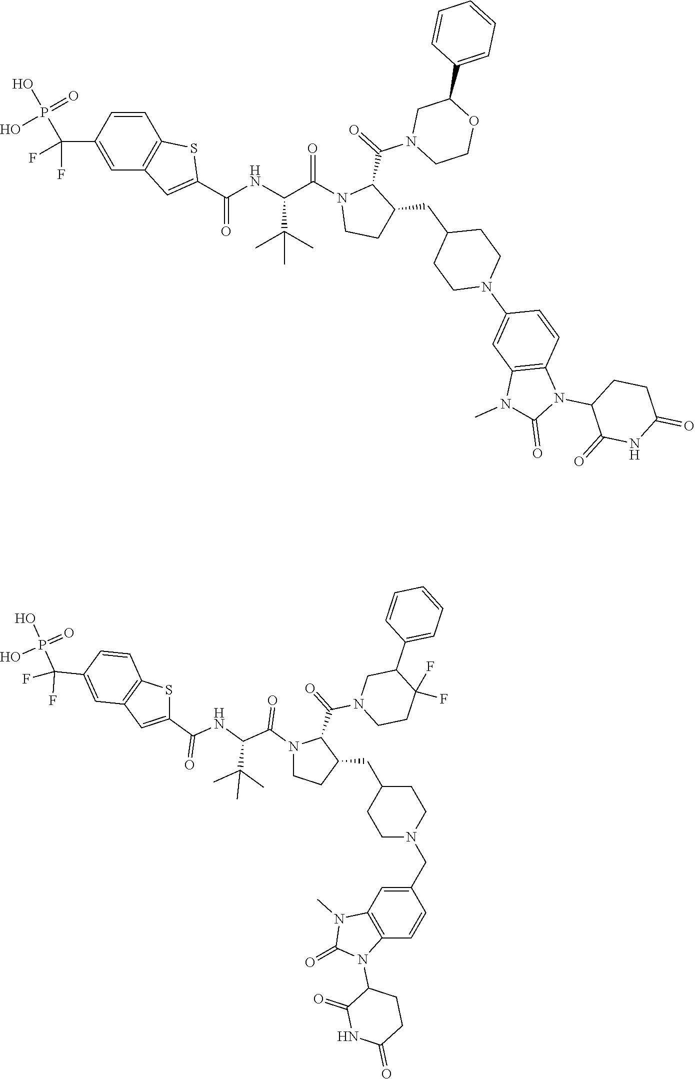 [SMILES: Cn1c(=O)n(C2CCC(=O)NC2=O)c2ccc(N3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21 ; Cn1c(=O)n(C2CCC(=O)NC2=O)c2ccc(CN3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCC(F)(F)C(c5ccccc5)C4)CC3)cc21]

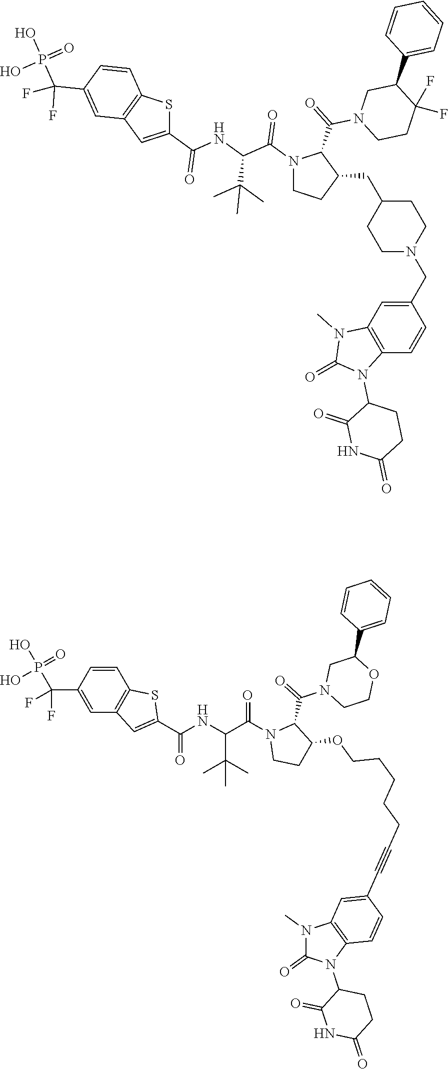 [SMILES: Cn1c(=O)n(C2CCC(=O)NC2=O)c2ccc(CN3CCC(C[C@@H]4CCN(C(=O)[C@@H](NC(=O)c5cc6cc(C(F)(F)P(=O)(O)O)ccc6s5)C(C)(C)C)[C@@H]4C(=O)N4CCC(F)(F)[C@H](c5ccccc5)C4)CC3)cc21 ; Cn1c(=O)n(C2CCC(=O)NC2=O)c2ccc(C#CCCCCCO[C@@H]3CCN(C(=O)C(NC(=O)c4cc5cc(C(F)(F)P(=O)(O)O)ccc5s4)C(C)(C)C)[C@@H]3C(=O)N3CCO[C@H](c4ccccc4)C3)cc21]

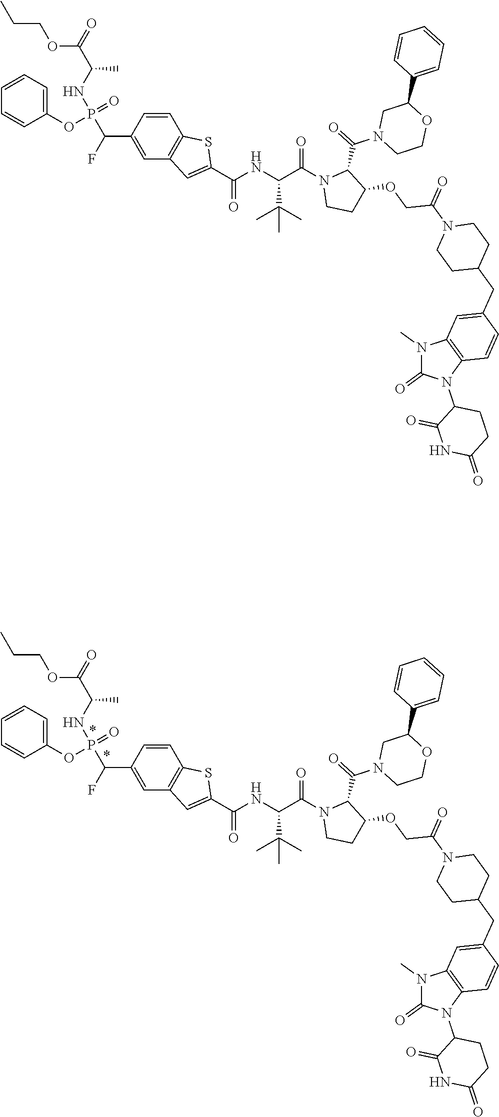 [SMILES: CCCOC(=O)[C@H](C)NP(=O)(Oc1ccccc1)C(F)c1ccc2sc(C(=O)N[C@H](C(=O)N3CC[C@@H](OCC(=O)N4CCC(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1 ; CCCOC(=O)[C@H](C)NP(=O)(Oc1ccccc1)C(F)c1ccc2sc(C(=O)N[C@H](C(=O)N3CC[C@@H](OCC(=O)N4CCC(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1]

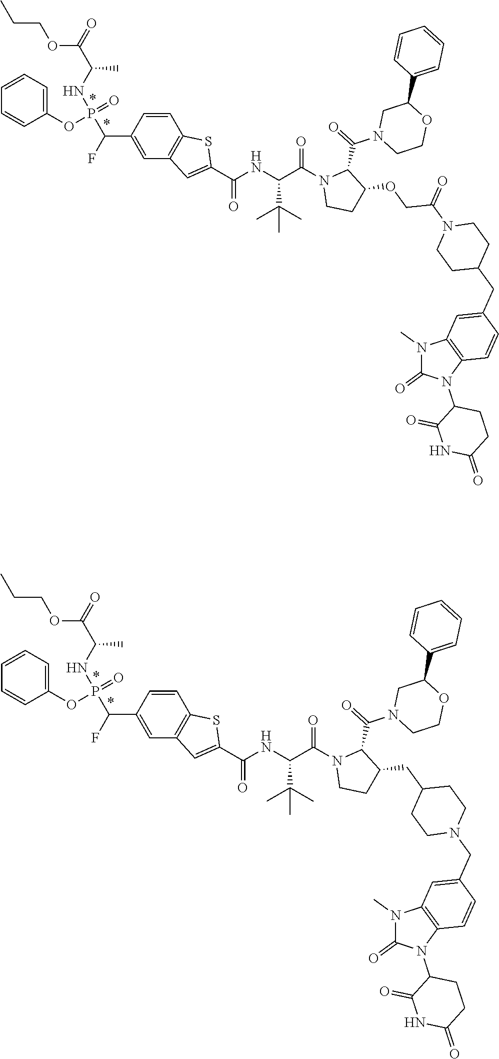 [SMILES: CCCOC(=O)[C@H](C)NP(=O)(Oc1ccccc1)C(F)c1ccc2sc(C(=O)N[C@H](C(=O)N3CC[C@@H](OCC(=O)N4CCC(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1 ; CCCOC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1]

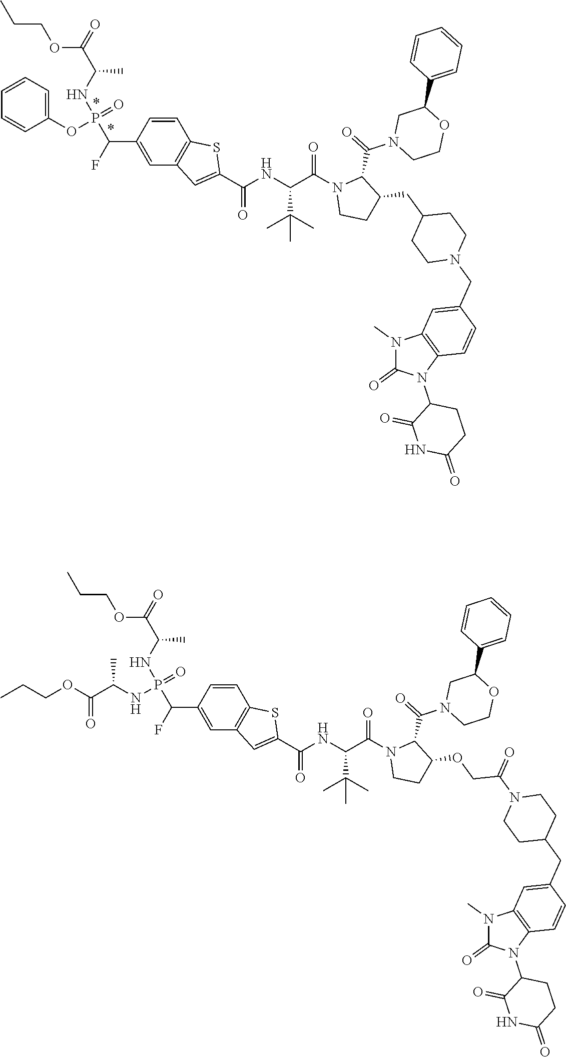 [SMILES: CCCOC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1 ; CCCOC(=O)[C@H](C)NP(=O)(N[C@@H](C)C(=O)OCCC)C(F)c1ccc2sc(C(=O)N[C@H](C(=O)N3CC[C@@H](OCC(=O)N4CCC(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1]

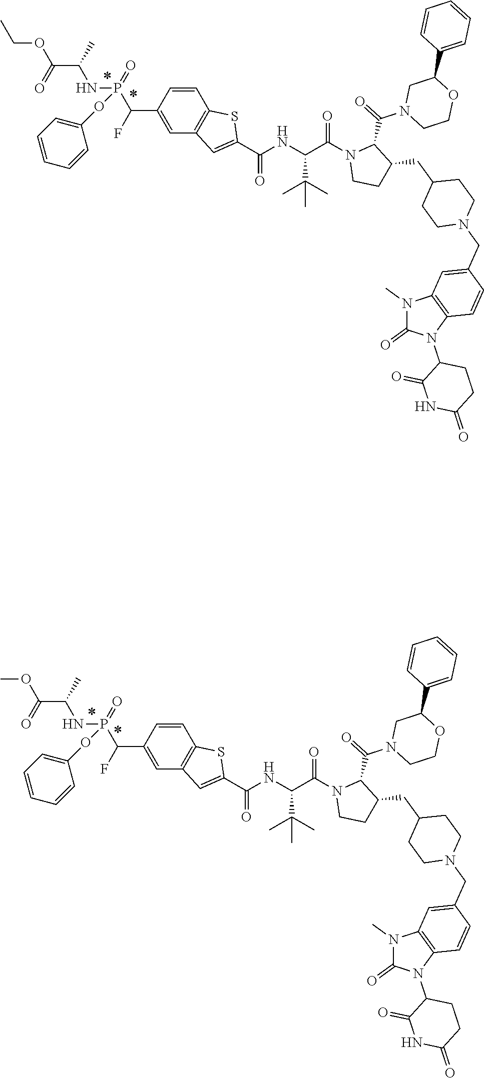 [SMILES: CCOC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1 ; COC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1]

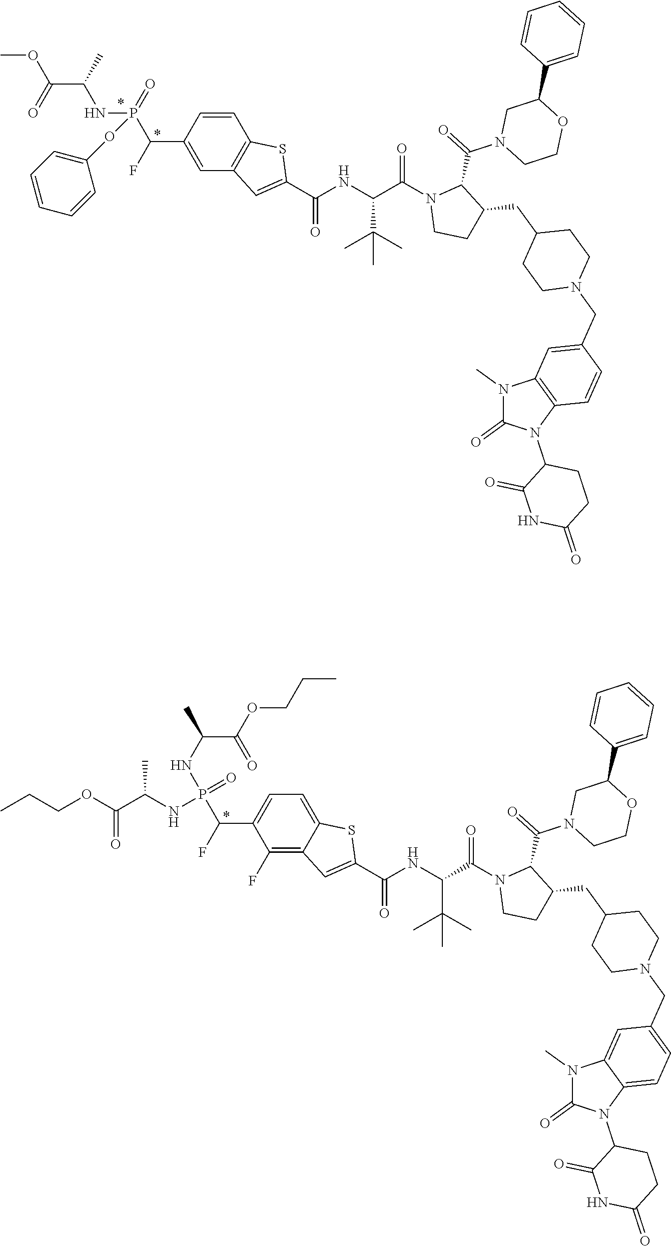 [SMILES: COC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1 ; CCCOC(=O)[C@H](C)NP(=O)(N[C@@H](C)C(=O)OCCC)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1F]

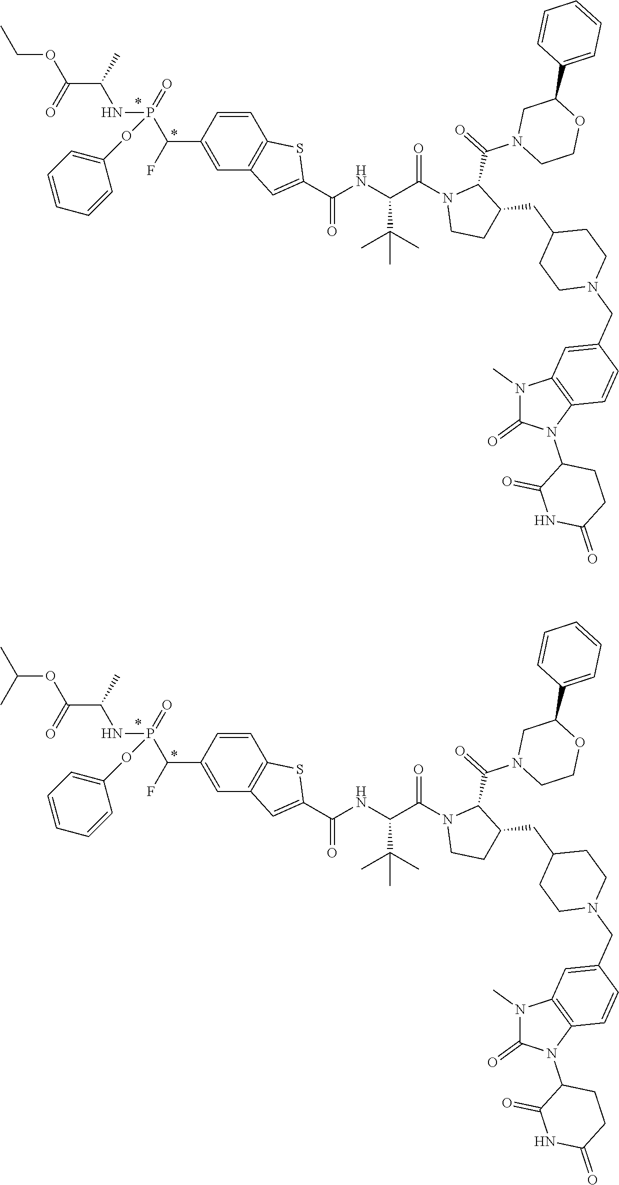 [SMILES: CCOC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1 ; CC(C)OC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1]

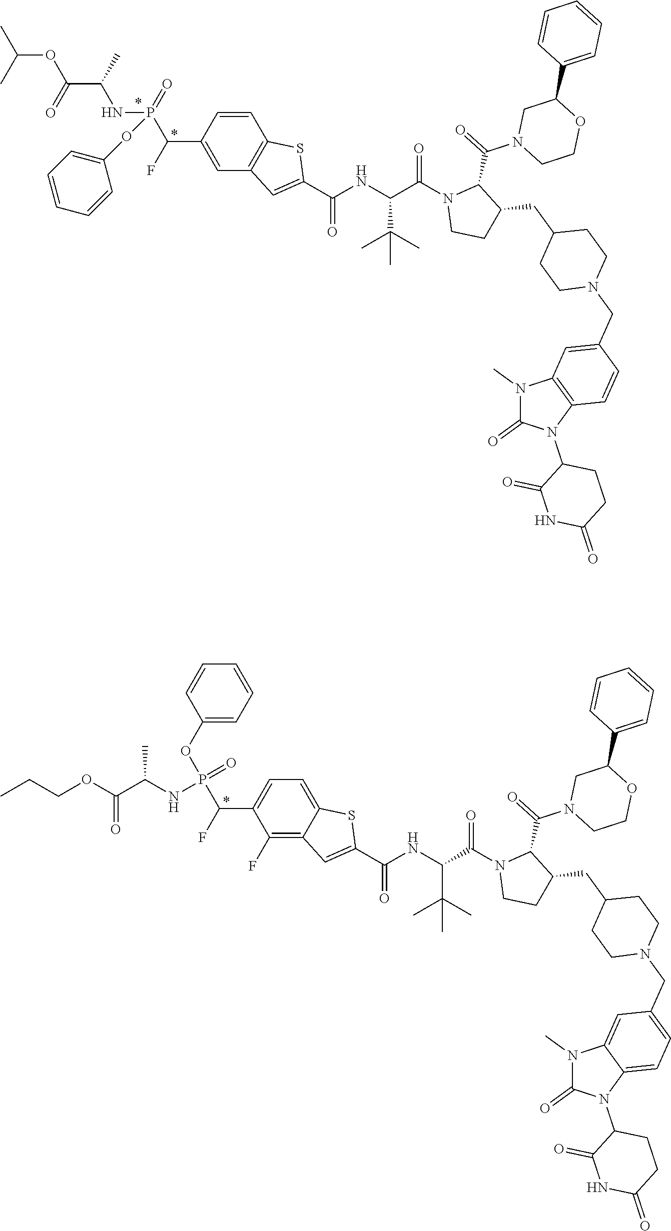 [SMILES: CC(C)OC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1 ; CCCOC(=O)[C@H](C)NP(=O)(Oc1ccccc1)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1F]

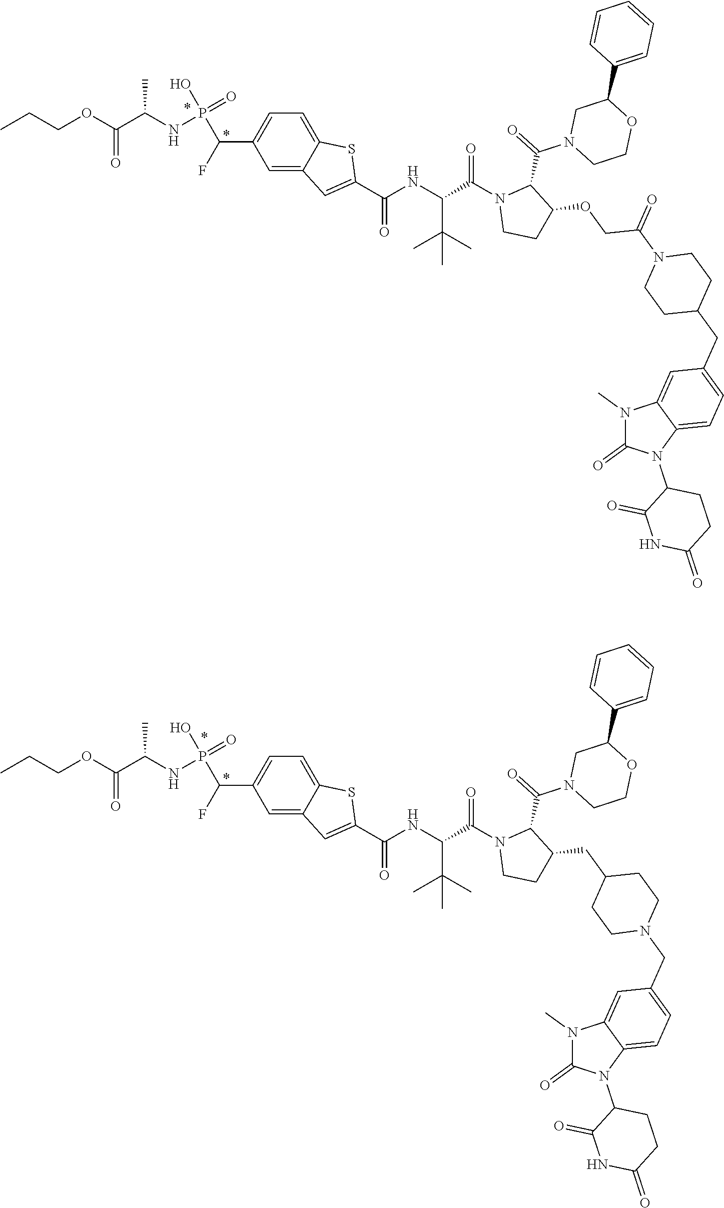 [SMILES: CCCOC(=O)[C@H](C)NP(=O)(O)C(F)c1ccc2sc(C(=O)N[C@H](C(=O)N3CC[C@@H](OCC(=O)N4CCC(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1 ; CCCOC(=O)[C@H](C)NP(=O)(O)C(F)c1ccc2sc(C(=O)N[C@H](C(=O)N3CC[C@@H](CC4CCN(Cc5ccc6c(c5)n(C)c(=O)n6C5CCC(=O)NC5=O)CC4)[C@H]3C(=O)N3CCO[C@H](c4ccccc4)C3)C(C)(C)C)cc2c1]

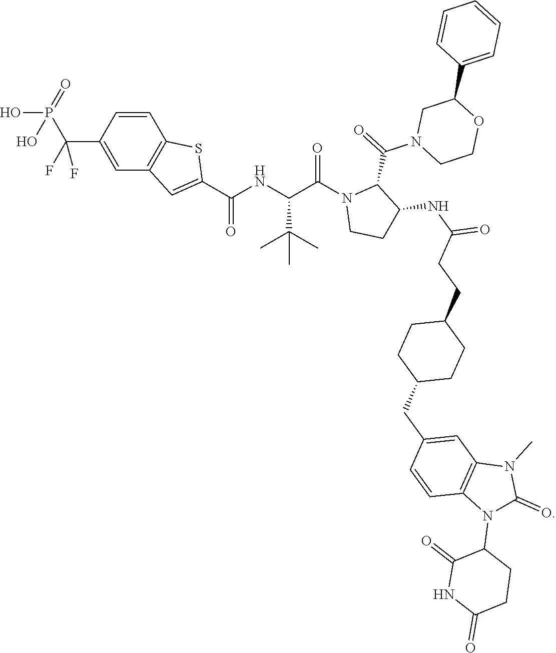 [SMILES: Cn1c(=O)n(C2CCC(=O)NC2=O)c2ccc(C[C@H]3CC[C@H](CCC(=O)N[C@@H]4CCN(C(=O)[C@@H](NC(=O)c5cc6cc(C(F)(F)P(=O)(O)O)ccc6s5)C(C)(C)C)[C@@H]4C(=O)N4CCO[C@H](c5ccccc5)C4)CC3)cc21]

31. A pharmaceutical composition comprising a compound of Formula J 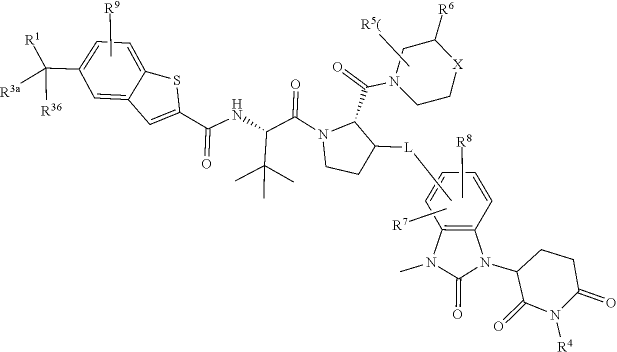
or a pharmaceutically acceptable salt thereof, wherein
R¹ is 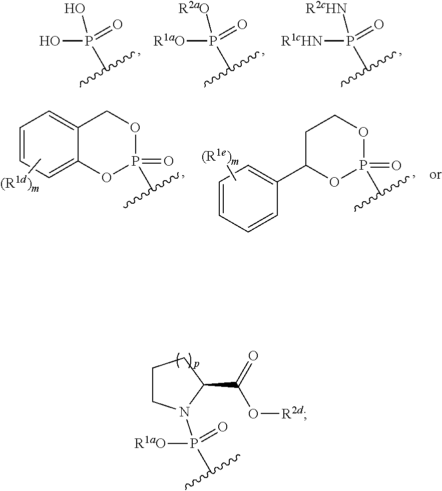
R^{1a} and R^{2a} are each independently C₁₋₆ alkyl, C₁₋₆ haloalkyl, C₁₋₆ alkylene-OC(O)—C₁₋₆ alkyl, C₁₋₆ alkylene-OC(O)O—C₁₋₆ alkyl, C₁₋₆ alkylene-SC(O)—C₁₋₆ alkyl, C₃₋₆ cycloalkyl, C₆₋₁₂ aryl, or 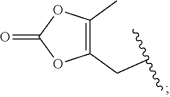 [SMILES: *Cc1oc(=O)oc1C]
R^{1b} is H, C₁₋₆ alkyl, C₁₋₆ haloalkyl, C₃₋₆ cycloalkyl or C₆₋₁₂ aryl;
R^{1c}, R^{2b}, and R^{2c} are each independently 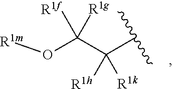
wherein
R^{1f} and R^{1g} are each independently H or C₁₋₆ alkyl, or R^{1f} and R^{1g} together are oxo,
R^{1h} and R^{1k}, are each independently H, C₁₋₆ alkyl, C₁₋₆ haloalkyl, C₂₋₆ alkoxyalkyl, or CH₂Ph, and
R^{1m} is C₁₋₆ alkyl, C₁₋₆ haloalkyl, C₂₋₆ alkoxyalkyl, —C(O)—C₁₋₆ alkyl, —C(O)O—C₁₋₆ alkyl, C₃₋₈ cycloalkyl, —CH₂—C₃₋₈ cycloalkyl, heterocycloalkyl, or —CH₂-heterocycloalkyl, wherein each heterocycloalkyl has 3 to 8 ring members and 1 to 3 heteroatoms each independently N, O, or S, provided that when R^{m} is —C(O)C₁₋₆ alkyl or —C(O)O—C₁₋₆ alkyl, then R^{1f} and R^{1g} are each independently H or C₁₋₆ alkyl;
R^{1d} and R^{1e} are each independently C₁₋₆ alkyl, C₁₋₆ alkoxy, C₂₋₆ alkoxyalkyl, halogen, C₁₋₆ haloalkyl, C₁₋₆ haloalkoxy, or CN;
R^{2d} is C₁₋₆ alkyl or C₁₋₆ haloalkyl;
R^{3a} and R^{3b} are each independently H, C₁₋₆ alkyl, halogen, or C₁₋₆ haloalkyl, or R^{3a} and R^{3b} together are oxo;
L is -L¹-L²-L³-L⁴-L⁵-;
L¹ is absent, CH₂, O, or N(L^{1a});
L^{1a} is hydrogen or C₁₋₃ alkyl;
L² is absent, C₁₋₆ alkylene, C₁₋₆ alkylene-C(O), CH₂C(O)N(L^{2a}), CH₂C(O)N(L^{2a})-C₁₋₆ alkylene, —C(O)—, or —C(O)—C₁₋₆ alkylene, provided that when L² is —C(O)— or —C(O)—C₁₋₆ alkylene, then L¹ is absent, CH₂, or N(L^{1a});
L^{2a} is hydrogen or C₁₋₆ alkyl;
L³ is absent, C₃₋₈ cycloalkylene, or a heterocycloalkylene having 4 to 11 ring members and 1 to 3 heteroatoms each independently N, O, or S, wherein each cycloalkylene and heterocycloalkylene is substituted with 0, 1, 2, or 3 R^{L3};
L⁴ is absent, C₁₋₆ alkylene, C₂₋₆ alkenylene, C₂₋₆ alkynylene, O, or C(O), wherein the alkylene is substituted with 0, 1, 2, or 3 R^{L4};
L⁵ is absent, C₁₋₆ alkylene, C₃₋₈ cycloalkylene, or a heterocycloalkylene having 4 to 11 ring members and 1 to 3 heteroatoms each independently N, O, or S, wherein each alkylene, cycloalkylene and heterocycloalkylene is substituted with 0, 1, 2, or 3 R^{L5}, and wherein no more than three of L¹, L², L³, L⁴, and L⁵ are absent;
each R^{L3}, R^{L4} and R^{L5} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
R⁴ is hydrogen, CH₂OC(O)—C₁₋₆ alkyl, CH₂OC(O)O—C₁₋₆ alkyl, or CH₂—OP(O)(OH)₂;
each R⁵ is independently halo, C₁₋₆ alkyl, or C₁₋₆ haloalkyl;
alternatively, where two R⁵ groups are bound to the same ring atom, such R⁵ groups can be taken together with the atom to which they are attached to form a C₃₋₅ cycloalkyl;
R⁶ is hydrogen, C₁₋₆ alkyl, C₁₋₆ alkoxy, C₆₋₁₀ aryl, heteroaryl having 5 to 10 ring members and 1 to 3 heteroatoms each independently N, O, or S, or —OR⁶a, wherein the aryl and heteroaryl is substituted with 0, 1, 2, or 3 R^{6b};
R^{6a} is C₆₋₁₀ aryl or heteroaryl having 5 to 10 ring members and 1 to 3 heteroatoms each independently N, O, or S, wherein the aryl and heteroaryl is substituted with 0, 1, 2, or 3 R^{6c};
each R^{6b} and R^{6c} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
X is —CH₂—, —CF₂—, —CH(CN)—, —O—, or —N(R^{X});
R^{X} is hydrogen or C₁₋₆ alkyl;
subscript m is 0, 1, 2, or 3;
subscript n is 0, 1, or 2, provided that when subscript n is 0, then X is —CH₂— or —CH(CN)—, and if subscript n is 1 or 2 then X is —O—, —CH₂—, or —N(R^{X});
subscript p is 0, 1, or 2;
subscript q is 0, 1, or 2; and
R⁷, R⁸, and R⁹ are each independently hydrogen or fluorine,
and a pharmaceutically acceptable excipient.

32. The pharmaceutical composition of claim 31, further comprising one or more additional therapeutic agents.

33. A method of treating an immune mediated disease or condition in a subject in need thereof, the method comprising administering to the subject a therapeutically effective amount of a compound of 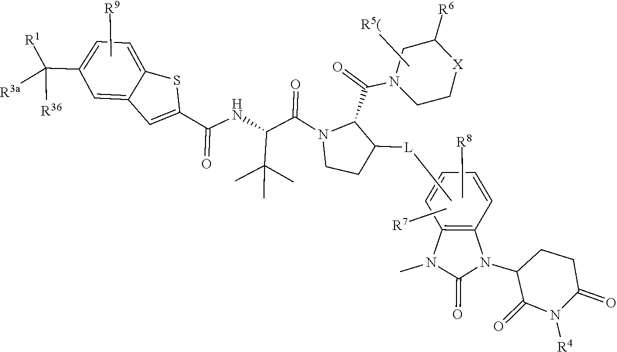
or a pharmaceutically acceptable salt thereof, wherein
R¹ is 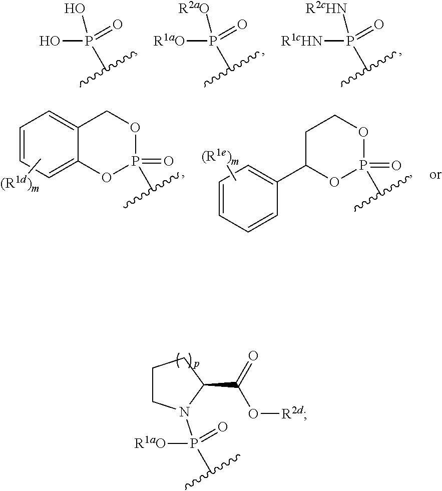
R^{1a} and R^{2a} are each independently C₁₋₆ alkyl, C₁₋₆ haloalkyl, C₁₋₆ alkylene-OC(O)—C₁₋₆ alkyl, C₁₋₆ alkylene-OC(O)O—C₁₋₆ alkyl, C₁₋₆ alkylene-SC(O)—C₁₋₆ alkyl, C₃₋₆ cycloalkyl, C₆₋₁₂ aryl, or 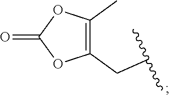 [SMILES: *Cc1oc(=O)oc1C]
R^{1b} is H, C₁₋₆ alkyl, C₁₋₆ haloalkyl, C₃₋₆ cycloalkyl or C₆₋₁₂ aryl;
R^{1c}, R^{2b}, and R^{2c} are each independently 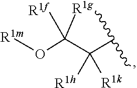
wherein
R^{1f} and R^{1g} are each independently H or C₁₋₆ alkyl, or R^{1f} and R^{1g} together are oxo,
R^{1h} and R^{1k}, are each independently H, C₁₋₆ alkyl, C₁₋₆ haloalkyl, C₂₋₆ alkoxyalkyl, or CH₂Ph, and
R^{1m} is C₁₋₆ alkyl, C₁₋₆ haloalkyl, C₂₋₆ alkoxyalkyl, —C(O)—C₁₋₆ alkyl, —C(O)O—C₁₋₆ alkyl, C₃₋₈ cycloalkyl, —CH₂—C₃₋₈ cycloalkyl, heterocycloalkyl, or —CH₂-heterocycloalkyl, wherein each heterocycloalkyl has 3 to 8 ring members and 1 to 3 heteroatoms each independently N, O, or S, provided that when R^{1m} is —C(O)C₁₋₆ alkyl or —C(O)O—C₁₋₆ alkyl, then R^{1f} and R^{1g} are each independently H or C₁₋₆ alkyl;
R^{1d} and R^{1e} are each independently C₁₋₆ alkyl, C₁₋₆ alkoxy, C₂₋₆ alkoxyalkyl, halogen, C₁₋₆ haloalkyl, C₁₋₆ haloalkoxy, or CN;
R^{2d} is C₁₋₆ alkyl or C₁₋₆ haloalkyl;
R^{3a} and R^{3b} are each independently H, C₁₋₆ alkyl, halogen, or C₁₋₆ haloalkyl, or R^{3a} and R^{3b} together are oxo;
L is -L¹-L²-L³-L⁴-L⁵-;
L¹ is absent, CH₂, O, or N(L^{1a});
L^{1a} is hydrogen or C₁₋₃ alkyl;
L² is absent, C₁₋₆ alkylene, C₁₋₆ alkylene-C(O), CH₂C(O)N(L^{2a}), CH₂C(O)N(L^{2a})-C₁₋₆ alkylene, —C(O)—, or —C(O)—C₁₋₆ alkylene, provided that when L² is —C(O)— or —C(O)—C₁₋₆ alkylene, then L¹ is absent, CH₂, or N(L^{1a});
L^{2a} is hydrogen or C₁₋₆ alkyl;
L³ is absent, C₃₋₈ cycloalkylene, or a heterocycloalkylene having 4 to 11 ring members and 1 to 3 heteroatoms each independently N, O, or S, wherein each cycloalkylene and heterocycloalkylene is substituted with 0, 1, 2, or 3 R^{L3};
L⁴ is absent, C₁₋₆ alkylene, C₂₋₆ alkenylene, C₂₋₆ alkynylene, O, or C(O), wherein the alkylene is substituted with 0, 1, 2, or 3 R^{L4};
L⁵ is absent, C₁₋₆ alkylene, C₃₋₈ cycloalkylene, or a heterocycloalkylene having 4 to 11 ring members and 1 to 3 heteroatoms each independently N, O, or S, wherein each alkylene, cycloalkylene and heterocycloalkylene is substituted with 0, 1, 2, or 3 R^{L5}, and wherein no more than three of L¹, L², L³, L⁴, and L⁵ are absent;
each R^{L3}, R^{L4} and R^{L5} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
R⁴ is hydrogen, CH₂OC(O)—C₁₋₆ alkyl, CH₂OC(O)O—C₁₋₆ alkyl, or CH₂—OP(O)(OH)₂;
each R⁵ is independently halo, C₁₋₆ alkyl, or C₁₋₆ haloalkyl;
alternatively, where two R⁵ groups are bound to the same ring atom, such R⁵ groups can be taken together with the atom to which they are attached to form a C₃₋₅ cycloalkyl;
R⁶ is hydrogen, C₁₋₆ alkyl, C₁₋₆ alkoxy, C₆₋₁₀ aryl, heteroaryl having 5 to 10 ring members and 1 to 3 heteroatoms each independently N, O, or S, or —OR^{6a}, wherein the aryl and heteroaryl is substituted with 0, 1, 2, or 3 R^{6b};
R^{6a} is C₆₋₁₀ aryl or heteroaryl having 5 to 10 ring members and 1 to 3 heteroatoms each independently N, O, or S, wherein the aryl and heteroaryl is substituted with 0, 1, 2, or 3 R^{6c};
each R^{6b} and R^{6c} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
X is —CH₂—, —CF₂—, —CH(CN)—, —O—, or —N(R^{X})—;
R^{X} is hydrogen or C₁₋₆ alkyl;
subscript m is 0, 1, 2, or 3;
subscript n is 0, 1, or 2, provided that when subscript n is 0, then X is —CH₂— or —CH(CN)—, and if subscript n is 1 or 2 then X is —O—, —CH₂—, or —N(R^{X})—;
subscript p is 0, 1, or 2;
subscript q is 0, 1, or 2; and
R⁷, R⁸, and R⁹ are each independently hydrogen or fluorine.

34. (canceled)

35. (canceled)

36. A method of treating a disease or condition mediated by a signal transducer and activator of transcription 6 (STAT6) protein activity in a subject in need thereof, the method comprising administering to the subject a therapeutically effective amount of a compound of Formula J 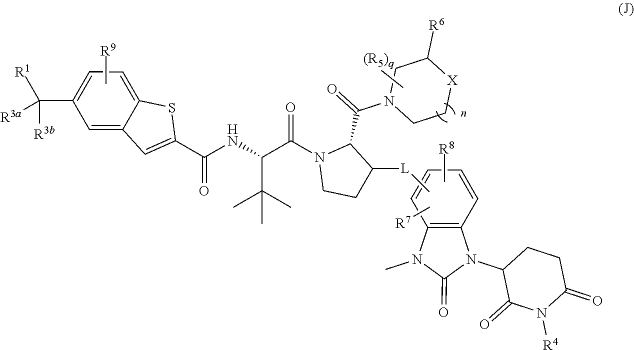
or a pharmaceutically acceptable salt thereof, wherein
R¹ is 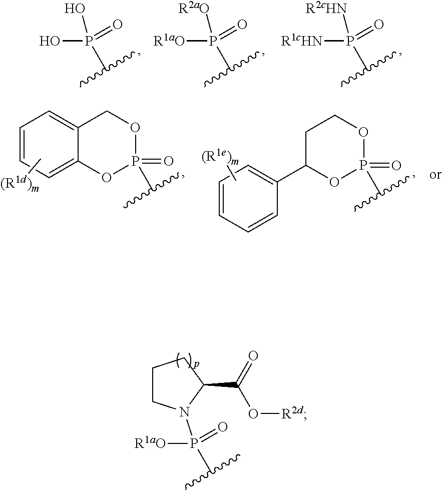
R^{1a} and R^{2a} are each independently C₁₋₆ alkyl, C₁₋₆ haloalkyl, C₁₋₆ alkylene-OC(O)—C₁₋₆ alkyl, C₁₋₆ alkylene-OC(O)O—C₁₋₆ alkyl, C₁₋₆ alkylene-SC(O)—C₁₋₆ alkyl, C₃₋₆ cycloalkyl, C₆₋₁₂ aryl, or 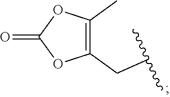 [SMILES: *Cc1oc(=O)oc1C]
R^{1b} is H, C₁₋₆ alkyl, C₁₋₆ haloalkyl, C₃₋₆ cycloalkyl or C₆₋₁₂ aryl;
R^{1c}, R^{2b}, and R^{2c} are each independently 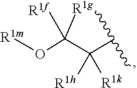
wherein
R^{1f} and R^{1g} are each independently H or C₁₋₆ alkyl, or R^{1f} and R^{1g} together are oxo,
R^{1h} and R^{1k}, are each independently H, C₁₋₆ alkyl, C₁₋₆ haloalkyl, C₂₋₆ alkoxyalkyl, or CH₂Ph, and
R^{1m} is C₁₋₆ alkyl, C₁₋₆ haloalkyl, C₂₋₆ alkoxyalkyl, —C(O)—C₁₋₆ alkyl, —C(O)O—C₁₋₆ alkyl, C₃₋₈ cycloalkyl, —CH₂—C₃₋₈ cycloalkyl, heterocycloalkyl, or —CH₂-heterocycloalkyl, wherein each heterocycloalkyl has 3 to 8 ring members and 1 to 3 heteroatoms each independently N, O, or S, provided that when R^{m} is —C(O)C₁₋₆ alkyl or —C(O)O—C₁₋₆ alkyl, then R^{1f} and R^{1g} are each independently H or C₁₋₆ alkyl;
R^{1d} and R^{1e} are each independently C₁₋₆ alkyl, C₁₋₆ alkoxy, C₂₋₆ alkoxyalkyl, halogen, C₁₋₆ haloalkyl, C₁₋₆ haloalkoxy, or CN;
R^{2d} is C₁₋₆ alkyl or C₁₋₆ haloalkyl;
R^{3a} and R^{3b} are each independently H, C₁₋₆ alkyl, halogen, or C₁₋₆ haloalkyl, or R^{3a} and R^{3b} together are oxo;
L is -L¹-L²-L³-L⁴-L⁵-;
L¹ is absent, CH₂, O, or N(L^{1a});
L^{1a} is hydrogen or C₁₋₃ alkyl;
L² is absent, C₁₋₆ alkylene, C₁₋₆ alkylene-C(O), CH₂C(O)N(L^{2a}), CH₂C(O)N(L^{2a})-C₁₋₆ alkylene, —C(O)—, or —C(O)—C₁₋₆ alkylene, provided that when L² is —C(O)— or —C(O)—C₁₋₆ alkylene, then L¹ is absent, CH₂, or N(L^{1a});
L^{2a} is hydrogen or C₁₋₆ alkyl;
L³ is absent, C₃₋₈ cycloalkylene, or a heterocycloalkylene having 4 to 11 ring members and 1 to 3 heteroatoms each independently N, O, or S, wherein each cycloalkylene and heterocycloalkylene is substituted with 0, 1, 2, or 3 R^{L3};
L⁴ is absent, C₁₋₆ alkylene, C₂₋₆ alkenylene, C₂₋₆ alkynylene, O, or C(O), wherein the alkylene is substituted with 0, 1, 2, or 3 R^{L4};
L⁵ is absent, C₁₋₆ alkylene, C₃₋₈ cycloalkylene, or a heterocycloalkylene having 4 to 11 ring members and 1 to 3 heteroatoms each independently N, O, or S, wherein each alkylene, cycloalkylene and heterocycloalkylene is substituted with 0, 1, 2, or 3 R^{L5}, and wherein no more than three of L¹, L², L³, L⁴, and L⁵ are absent;
each R^{L3}, R^{L4} and R^{L5} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
R⁴ is hydrogen, CH₂OC(O)—C₁₋₆ alkyl, CH₂OC(O)O—C₁₋₆ alkyl, or CH₂—OP(O)(OH)₂;
each R⁵ is independently halo, C₁₋₆ alkyl, or C₁₋₆ haloalkyl;
alternatively, where two R⁵ groups are bound to the same ring atom, such R⁵ groups can be taken together with the atom to which they are attached to form a C₃₋₅ cycloalkyl;
R⁶ is hydrogen, C₁₋₆ alkyl, C₁₋₆ alkoxy, C₆₋₁₀ aryl, heteroaryl having 5 to 10 ring members and 1 to 3 heteroatoms each independently N, O, or S, or —OR^{6a}, wherein the aryl and heteroaryl is substituted with 0, 1, 2, or 3 R^{6b};
R^{6a} is C₆₋₁₀ aryl or heteroaryl having 5 to 10 ring members and 1 to 3 heteroatoms each independently N, O, or S, wherein the aryl and heteroaryl is substituted with 0, 1, 2, or 3 R^{6c};
each R^{6b} and R^{6c} is independently C₁₋₆ alkyl, C₁₋₆ alkoxy, C₂₋₆ alkoxyalkyl, halogen, C₁₋₆ haloalkyl, C₁₋₆ haloalkoxy, or —CN;
X is —CH₂—, —CF₂—, —CH(CN)—, —O—, or —N(R^{X})—;
R^{X} is hydrogen or C₁₋₆ alkyl;
subscript m is 0, 1, 2, or 3;
subscript n is 0, 1, or 2, provided that when subscript n is 0, then X is —CH₂— or —CH(CN)—, and if subscript n is 1 or 2 then X is —O—, —CH₂—, or —N(R^{X})—;
subscript p is 0, 1, or 2;
subscript q is 0, 1, or 2; and
R⁷, R⁸, and R⁹ are each independently hydrogen or fluorine.

37. The method of claim 36, wherein the STAT6-mediated disorder or disease is from the class of diseases or rheumatology, gastroenterology, pulmonology, hepatology, nephrology, dermatology or an allergic disease.

38. The method of claim 36, wherein the STAT6-mediated disorder or disease is rheumatoid arthritis (RA), systemic lupus erythematosus (SLE), lupus nephritis (LN), osteoarthritis (OA), ulcerative colitis (UC), Crohn's disease (CD), idiopathic pulmonary fibrosis (IPF), interstitial lung disease (ILD), metabolic dysfunction-associated steatohepatitis (MASH), Diabetic kidney disease (DKD) (diabetic nephropathy), or atopic dermatitis (AD).

39. The method of claim 36, wherein the STAT6-mediated disorder or disease is atopic dermatitis, contact dermatitis, vitiligo, alopecia areata, acne, psoriasis, dermatomyositis, scleroderma, or morphea.

40. The method of claim 36, wherein the STAT6-mediated disorder or disease is atopic dermatitis.

41. (canceled)

42. (canceled)

43. (canceled)

44. (canceled)

45. (canceled)
